(19) United States
(12) Patent Application Publication
(10) Pub. No.: US 2025/0171754 A1
(43) Pub. Date: May 29, 2025

(54) CRISPR-CAS9 COMPOSITIONS AND METHODS WITH A NOVEL CAS9 PROTEIN FOR GENOME EDITING AND GENE REGULATION

(71) Applicants: Duke University, Durham, NC (US); North Carolina State University, Raleigh, NC (US)

(72) Inventors: Charles A. Gersbach, Chapel Hill, NC (US); Gabriel Butterfield, Durham, NC (US); Dahlia Rohm, Durham, NC (US); Rodolphe Barrangou, Raleigh, NC (US); Avery Roberts, Raleigh, NC (US); Matthew Nethery, Raleigh, NC (US)

(21) Appl. No.: 18/840,829

(22) PCT Filed: Feb. 24, 2023
(86) PCT No.: PCT/US2023/063296

§ 371 Date: Aug. 22, 2024

Related U.S. Application Data
(60) Provisional application No. 63/339,316, filed on May 6, 2022.
(60) Provisional application No. 63/325,037, filed on Mar. 29, 2022.
(60) Provisional application No. 63/314,183, filed on Feb. 25, 2022.

(51) Int. Cl. C12N 9/22; C07K 14/47; C12N 15/11; C12N 15/90

(52) U.S. Cl. CPC C12N 9/22; C07K 14/4703; C12N 15/11; C12N 15/907; C07K 2319/00; C12N 2310/20

(57) ABSTRACT

Disclosed herein is a novel Cas9 protein. Further described herein are fusion proteins, compositions, and methods comprising the same. The novel Cas9 protein may be used, for example, in compositions and methods for modulating expression of a gene, for correcting a mutant gene, and for treating a disease.

### CROSS-REFERENCE TO RELATED APPLICATIONS

This application claims priority to U.S. Provisional Patent Application No. 63/314,183, filed Feb. 25, 2022, U.S. Provisional Patent Application No. 63/325,037, filed Mar. 29, 2022, and U.S. Provisional Patent Application No. 63/339,316, filed May 6, 2022, the entire contents of each of which are hereby incorporated by reference.

### STATEMENT REGARDING FEDERALLY SPONSORED RESEARCH

This invention was made with government support under grant U01AI146356 awarded by the National Institutes of Health. The government has certain rights in the invention.

### FIELD

This disclosure relates to a novel Cas9 protein, novel Cas9 fusion proteins, novel CRISPR-Cas9 compositions, and methods of using the same for genome editing and gene regulation.

### INTRODUCTION

Synthetic transcription factors have been engineered to control gene expression for many different medical and scientific applications in mammalian systems, including stimulating tissue regeneration, drug screening, compensating for genetic defects, activating silenced tumor suppressors, controlling stem cell differentiation, performing genetic screens, and creating synthetic gene circuits. These transcription factors can target promoters or enhancers of endogenous genes or be purposefully designed to recognize sequences orthogonal to mammalian genomes for transgene regulation. The most common strategies for engineering novel transcription factors targeted to user-defined sequences have been based on the programmable DNA-binding domains of zinc finger proteins and transcription-activator like effectors (TALEs). Both of these approaches involve applying the principles of protein-DNA interactions of these domains to engineer new proteins with unique DNA-binding specificity. Although these methods have been widely successful for many applications, the protein engineering necessary for manipulating protein-DNA interactions can be laborious and require specialized expertise.

Additionally, these new proteins are not always effective. The reasons for this are not yet known but may be related to the effects of epigenetic modifications and chromatin state on protein binding to the genomic target site. In addition, there are challenges in ensuring that these new proteins, as well as other components, are delivered to each cell. Existing methods for delivering these new proteins and their multiple components include delivery to cells on separate plasmids or vectors, which leads to highly variable expression levels in each cell due to differences in copy number. Additionally, gene activation following transfection is transient due to dilution of plasmid DNA, and temporary gene expression may not be sufficient for inducing therapeutic effects. Furthermore, this approach is not amenable to cell types that are not easily transfected. Thus, another limitation of these new proteins is the potency of transcriptional activation.

Site-specific nucleases can be used to introduce site-specific double strand breaks at targeted genomic loci. This DNA cleavage stimulates the natural DNA-repair machinery, leading to one of two possible repair pathways. In the absence of a donor template, the break will be repaired by non-homologous end joining (NHEJ), an error-prone repair pathway that leads to small insertions or deletions of DNA. This method can be used to intentionally disrupt, delete, or alter the reading frame of targeted gene sequences. However, if a donor template is provided along with the nucleases, then the cellular machinery will repair the break by homologous recombination, which is enhanced several orders of magnitude in the presence of DNA cleavage. This method can be used to introduce specific changes in the DNA sequence at target sites. Engineered nucleases have been used for gene editing in a variety of human stem cells and cell lines, and for gene editing in the mouse liver. However, the major hurdle for implementation of these technologies is delivery to particular tissues in vivo in a way that is effective, efficient, and facilitates successful genome modification.

### SUMMARY

In an aspect, the disclosure relates to a Clustered Regularly Interspaced Short Palindromic Repeats associated (Cas) protein. The Cas protein may comprise an amino acid sequence having at least 80%, 85%, 90%, 95%, or 98% or greater identity to at least one of SEQ ID NOs: 57, 241, 243, 245, 247, 249, 251, 235, or 223, or any fragment thereof. The Cas protein may be from Streptococcus uberis, Streptococcus agalactiae, Streptococcus gallolyticus, Streptococcus iniae, Streptococcus lutetiensis, Streptococcus mutans, Streptococcus parauberis, Streptococcus dysgalactiae, or Streptococcus parasanguinis. In some embodiments, the Cas protein comprises an amino acid sequence having one, two, three, four, five or more changes selected from amino acid substitutions, insertions, or deletions, relative to SEQ ID NO: 57, or any fragment thereof, or the Cas protein comprises the amino acid sequence of SEQ ID NO: 57, or the Cas protein is encoded by a polynucleotide comprising a sequence having at least 80%, 85%, 90%, 95%, or 98% or greater identity to SEQ ID NO: 58, or any fragment thereof, or the Cas protein is encoded by a polynucleotide comprising a sequence having one, two, three, four, five or more changes selected from nucleotide substitutions, insertions, or deletions, relative to SEQ ID NO: 58, or any fragment thereof, or the Cas protein is encoded by a polynucleotide comprising the sequence of SEQ ID NO: 58. In some embodiments, the Cas protein comprises an amino acid sequence having one, two, three, four, five or more changes selected from amino acid substitutions, insertions, or deletions, relative to SEQ ID NO: 223, or any fragment thereof, or the Cas protein comprises the amino acid sequence of SEQ ID NO: 223, or the Cas protein is encoded by a polynucleotide comprising a sequence having at least 80%, 85%, 90%, 95%, or 98% or greater identity to SEQ ID NO: 224, or any fragment thereof, or the Cas protein is encoded by a polynucleotide comprising a sequence having one, two, three, four, five or more changes selected from nucleotide substitutions, insertions, or deletions, relative to SEQ ID NO: 224, or any fragment thereof, or the Cas protein is encoded by a polynucleotide comprising the sequence of SEQ ID NO: 224. In some embodiments, the Cas protein comprises an amino acid sequence having one, two, three, four, five or more changes selected from amino acid substitutions, insertions, or deletions, relative to SEQ ID NO: 241, or any fragment thereof, or the Cas protein comprises the amino acid sequence of SEQ ID NO: 241, or the Cas protein is encoded by a polynucleotide comprising a sequence having at least 80%, 85%, 90%, 95%, or 98% or greater identity to SEQ ID NO: 242, or any fragment thereof, or the Cas protein is encoded by a polynucleotide comprising a sequence having one, two, three, four, five or more changes selected from nucleotide substitutions, insertions, or deletions, relative to SEQ ID NO: 242, or any fragment thereof, or the Cas protein is encoded by a polynucleotide comprising the sequence of SEQ ID NO: 242. In some embodiments, the Cas protein comprises an amino acid sequence having one, two, three, four, five or more changes selected from amino acid substitutions, insertions, or deletions, relative to SEQ ID NO: 243, or any fragment thereof, or the Cas protein comprises the amino acid sequence of SEQ ID NO: 243, or the Cas protein is encoded by a polynucleotide comprising a sequence having at least 80%, 85%, 90%, 95%, or 98% or greater identity to SEQ ID NO: 244, or any fragment thereof, or the Cas protein is encoded by a polynucleotide comprising a sequence having one, two, three, four, five or more changes selected from nucleotide substitutions, insertions, or deletions, relative to SEQ ID NO: 244, or any fragment thereof, or the Cas protein is encoded by a polynucleotide comprising the sequence of SEQ ID NO: 244. In some embodiments, the Cas protein comprises an amino acid sequence having one, two, three, four, five or more changes selected from amino acid substitutions, insertions, or deletions, relative to SEQ ID NO: 245, or any fragment thereof, or the Cas protein comprises the amino acid sequence of SEQ ID NO: 245, or the Cas protein is encoded by a polynucleotide comprising a sequence having at least 80%, 85%, 90%, 95%, or 98% or greater identity to SEQ ID NO: 246, or any fragment thereof, or the Cas protein is encoded by a polynucleotide comprising a sequence having one, two, three, four, five or more changes selected from nucleotide substitutions, insertions, or deletions, relative to SEQ ID NO: 246, or any fragment thereof, or the Cas protein is encoded by a polynucleotide comprising the sequence of SEQ ID NO: 246. In some embodiments, the Cas protein comprises an amino acid sequence having one, two, three, four, five or more changes selected from amino acid substitutions, insertions, or deletions, relative to SEQ ID NO: 247, or any fragment thereof, or the Cas protein comprises the amino acid sequence of SEQ ID NO: 247, or the Cas protein is encoded by a polynucleotide comprising a sequence having at least 80%, 85%, 90%, 95%, or 98% or greater identity to SEQ ID NO: 248, or any fragment thereof, or the Cas protein is encoded by a polynucleotide comprising a sequence having one, two, three, four, five or more changes selected from nucleotide substitutions, insertions, or deletions, relative to SEQ ID NO: 248, or any fragment thereof, or the Cas protein is encoded by a polynucleotide comprising the sequence of SEQ ID NO: 248. In some embodiments, the Cas protein comprises an amino acid sequence having one, two, three, four, five or more changes selected from amino acid substitutions, insertions, or deletions, relative to SEQ ID NO: 249, or any fragment thereof, or the Cas protein comprises the amino acid sequence of SEQ ID NO: 249, or the Cas protein is encoded by a polynucleotide comprising a sequence having at least 80%, 85%, 90%, 95%, or 98% or greater identity to SEQ ID NO: 250, or any fragment thereof, or the Cas protein is encoded by a polynucleotide comprising a sequence having one, two, three, four, five or more changes selected from nucleotide substitutions, insertions, or deletions, relative to SEQ ID NO: 250, or any fragment thereof, or the Cas protein is encoded by a polynucleotide comprising the sequence of SEQ ID NO: 250. In some embodiments, the Cas protein comprises an amino acid sequence having one, two, three, four, five or more changes selected from amino acid substitutions, insertions, or deletions, relative to SEQ ID NO: 251, or any fragment thereof, or the Cas protein comprises the amino acid sequence of SEQ ID NO: 251, or the Cas protein is encoded by a polynucleotide comprising a sequence having at least 80%, 85%, 90%, 95%, or 98% or greater identity to SEQ ID NO: 252, or any fragment thereof, or the Cas protein is encoded by a polynucleotide comprising a sequence having one, two, three, four, five or more changes selected from nucleotide substitutions, insertions, or deletions, relative to SEQ ID NO: 252, or any fragment thereof, or the Cas protein is encoded by a polynucleotide comprising the sequence of SEQ ID NO: 252. In some embodiments, the Cas protein comprises an amino acid sequence having one, two, three, four, five or more changes selected from amino acid substitutions, insertions, or deletions, relative to SEQ ID NO: 235, or any fragment thereof, or the Cas protein comprises the amino acid sequence of SEQ ID NO: 235, or the Cas protein is encoded by a polynucleotide comprising a sequence having at least 80%, 85%, 90%, 95%, or 98% or greater identity to SEQ ID NO: 236, or any fragment thereof, or the Cas protein is encoded by a polynucleotide comprising a sequence having one, two, three, four, five or more changes selected from nucleotide substitutions, insertions, or deletions, relative to SEQ ID NO: 236, or any fragment thereof, or the Cas protein is encoded by a polynucleotide comprising the sequence of SEQ ID NO: 236. In some embodiments, the Cas protein comprises at least one amino acid mutation that knocks out nuclease activity of the Cas protein. In some embodiments, the at least one amino acid mutation is at least one of D10A, H600A, H845A, H599A, H840A, H604A, H839A, and D9A. In some embodiments, the Cas protein comprises an amino acid sequence having at least 80%, 85%, 90%, 95%, or 98% or greater identity to at least one of SEQ ID NOs: 59, 193, 197, 201, 205, 209, 213, 237, 225, or any fragment thereof. In some embodiments, the Cas protein comprises an amino acid sequence having one, two, three, four, five or more changes selected from amino acid substitutions, insertions, or deletions, relative to at least one of SEQ ID NOs: 59, 193, 197, 201, 205, 209, 213, 237, 225, or any fragment thereof. In some embodiments, the Cas protein comprises the amino acid sequence of at least one of SEQ ID NOs: 59, 193, 197, 201, 205, 209, 213, 237, or 225. In some embodiments, the Cas protein is encoded by a polynucleotide comprising a sequence having at least 80%, 85%, 90%, 95%, or 98% or greater identity to at least one of SEQ ID NOs: 60, 194, 198, 202, 206, 210, 214, 238, 226, or any fragment thereof. In some embodiments, the Cas protein is encoded by a polynucleotide comprising a sequence having one, two, three, four, five or more changes selected from nucleotide substitutions, insertions, or deletions, relative to at least one of SEQ ID NOs: 60, 194, 198, 202, 206, 210, 214, 238, 226, or any fragment thereof. In some embodiments, the Cas protein is encoded by a polynucleotide comprising the sequence of at least one of SEQ ID NOs: 60, 194, 198, 202, 206, 210, 214, 238, or 226. In some embodiments, the Cas protein recognizes a PAM sequence of AATA (SEQ ID NO: 71), NNA(A/G)TAN (SEQ ID NO: 273), NNAATA (SEQ ID NO: 274), NNG(T/C)(G/A)AN (SEQ ID NO: 275), NNGTAAA (SEQ ID NO: 276), NNGGNNN (SEQ ID NO: 277), NGG (SEQ ID NO: 2), NNAAAAN (SEQ ID NO: 278), NNAAAAA (SEQ ID NO: 279), NNGGNTN (SEQ ID NO: 280), NNAA(A/G)GN (SEQ ID NO: 281), and/or NNAAAG (SEQ ID NO: 282). In a further aspect, the disclosure relates to a fusion protein comprising two heterologous polypeptide domains, wherein the first polypeptide domain comprises a Cas protein as detailed herein, and wherein the second polypeptide domain has an activity selected from the group consisting of transcription activation activity, transcription repression activity, transcription release factor activity, histone modification activity, nuclease activity, nucleic acid association activity, methylase activity, and demethylase activity, or a combination thereof. In some embodiments, the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4X repressor, Mxil repressor, SUV39H1, SUV39H2, G9A, ESET/SETBD1, Cir4, Su (var) 3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3. In some embodiments, the second polypeptide domain has transcription repression activity. In some embodiments, the second polypeptide domain comprises KRAB. In some embodiments, the KRAB comprises an amino acid sequence having at least 80%, 85%, 90%, 95%, or 98% or greater identity to SEQ ID NO: 45, or comprises an amino acid sequence having one, two, three, four, five or more changes selected from amino acid substitutions, insertions, or deletions, relative to SEQ ID NO: 45, or comprises the amino acid sequence of SEQ ID NO: 45, or is encoded by a polynucleotide comprising a sequence having at least 80%, 85%, 90%, 95%, or 98% or greater identity to SEQ ID NO: 46, or is encoded by a polynucleotide comprising a sequence having one, two, three, four, five or more changes selected from nucleotide substitutions, insertions, or deletions, relative to SEQ ID NO: 46 or is encoded by a polynucleotide comprising the sequence of SEQ ID NO: 46, or any fragment thereof. In some embodiments, the fusion protein comprises an amino acid sequence having at least 80%, 85%, 90%, 95%, or 98% or greater identity to at least one of SEQ ID NOs: 61, 217, 218, 219, 220, 221, 222, 239, 227, or comprises an amino acid sequence having one, two, three, four, five or more changes selected from amino acid substitutions, insertions, or deletions, relative to at least one of SEQ ID NOs: 61, 217, 218, 219, 220, 221, 222, 239, 227, or comprises the amino acid sequence of at least one of SEQ ID NOs: 61, 217, 218, 219, 220, 221, 222, 239, 227, or is encoded by a polynucleotide comprising a sequence having at least 80%, 85%, 90%, 95%, or 98% or greater identity to SEQ ID NO: 62 or 240 or 228, or is encoded by a polynucleotide comprising a sequence having one, two, three, four, five or more changes selected from nucleotide substitutions, insertions, or deletions, relative to SEQ ID NO: 62 or 240 or 228, or is encoded by a polynucleotide comprising the sequence of SEQ ID NO: 62 or 240 or 228, or any fragment thereof. In some embodiments, the second polypeptide domain has transcription activation activity. In some embodiments, the second polypeptide domain comprises p300 or a fragment thereof or VP64 or a fragment thereof. In some embodiments, the p300 or a fragment thereof comprises an amino acid sequence having at least 80%, 85%, 90%, 95%, or 98% or greater identity to SEQ ID NO: 41 or 42, or comprises an amino acid sequence having one, two, three, four, five or more changes selected from amino acid substitutions, insertions, or deletions, relative to SEQ ID NO: 41 or 42, or comprises the amino acid sequence of SEQ ID NO: 41 or 42, or any fragment thereof. In some embodiments, the fusion protein comprises an amino acid sequence having at least 80%, 85%, 90%, 95%, or 98% or greater identity to at least one of SEQ ID NOs: 253, 259, 263, 265, 267, 261, 269, 271, or 229, or comprises an amino acid sequence having one, two, three, four, five or more changes selected from amino acid substitutions, insertions, or deletions, relative to at least one of SEQ ID NOs: 253, 259, 263, 265, 267, 261, 269, 271, or 229, or comprises the amino acid sequence of at least one of SEQ ID NOs: 253, 259, 263, 265, 267, 261, 269, 271, or 229, or is encoded by a polynucleotide comprising a sequence having at least 80%, 85%, 90%, 95%, or 98% or greater identity to at least one of SEQ ID NO: 254, 260, 264, 266, 268, 262, 270, 272, or 230, or is encoded by a polynucleotide comprising a sequence having one, two, three, four, five or more changes selected from nucleotide substitutions, insertions, or deletions, relative to at least one of SEQ ID NO: 254, 260, 264, 266, 268, 262, 270, 272, or 230, or is encoded by a polynucleotide comprising the sequence of at least one of SEQ ID NO: 254, 260, 264, 266, 268, 262, 270, 272, or 230, or any fragment thereof.

Another aspect of the disclosure provides a DNA targeting composition comprising: a Cas protein as detailed herein or a fusion protein as detailed herein; and at least one guide RNA (gRNA) that targets the Cas protein to a target region of a target gene. In some embodiments, the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene. In some embodiments, the gRNA targets the Cas protein to a promoter of the target gene. In some embodiments, the target region is located between about 1 to about 1000 base pairs upstream of a transcription start site of the target gene. In some embodiments, the DNA targeting composition comprises two or more gRNAs, each gRNA binding to a different target region. In some embodiments, the at least one gRNA comprises the sequence of SEQ ID NO: 69 or 67 or is encoded by or targets a sequence comprising SEQ ID NO: 70 or 68. In some embodiments, the at least one gRNA comprises a sequence selected from SEQ ID NOs: 195, 199, 203, 207, 211, 215, or is encoded by or targets a polynucleotide comprising a sequence selected from SEQ ID NOs: 196, 200, 204, 208, 212, 216. In some embodiments, the at least one gRNA comprises a sequence selected from SEQ ID NOs: 91-94, 100-103, 108-122, 158-192, or is encoded by or targets a polynucleotide comprising a sequence selected from SEQ ID NOs: 76-90, 96-99, 123-157.

Another aspect of the disclosure provides an isolated polynucleotide sequence encoding a Cas protein as detailed herein or a fusion protein as detailed herein, or a DNA targeting composition as detailed herein.

Another aspect of the disclosure provides a vector comprising an isolated polynucleotide sequence as detailed herein. In some embodiments, the vector is an adeno-associated virus (AAV) vector.

Another aspect of the disclosure provides a cell comprising a DNA targeting composition of as detailed herein, or an isolated polynucleotide sequence as detailed herein, or a vector as detailed herein, or a combination thereof.

Another aspect of the disclosure provides a pharmaceutical composition comprising: a DNA targeting composition as detailed herein, or an isolated polynucleotide sequence as detailed herein, or a vector as detailed herein, or a combination thereof.

Another aspect of the disclosure provides a method of modulating expression of a gene in a cell or in a subject. The method may include administering to the cell or the subject a DNA targeting composition as detailed herein, or an isolated polynucleotide sequence as detailed herein, or a vector as detailed herein, or a pharmaceutical composition as detailed herein, or a combination thereof. In some embodiments, the expression of the gene is increased relative to a control. In some embodiments, the expression of the gene is decreased relative to a control. In some embodiments, the gene comprises the dystrophin gene.

Another aspect of the disclosure provides a method of correcting a mutant gene in a cell. The method may include administering to the cell or the subject a DNA targeting composition as detailed herein, or an isolated polynucleotide sequence as detailed herein, or a vector as detailed herein, or a pharmaceutical composition as detailed herein, or a combination thereof. In some embodiments, the method further includes administering to the cell or subject a donor DNA. In some embodiments, correcting a mutant gene comprises deleting, rearranging, or replacing the mutant gene. In some embodiments, the gene comprises the dystrophin gene.

Another aspect of the disclosure provides a method of treating a disease in a subject. The method may include administering to the subject a DNA targeting composition as detailed herein, or an isolated polynucleotide sequence as detailed herein, or a vector as detailed herein, or a cell as detailed herein, or a pharmaceutical composition as detailed herein, or a combination thereof. In some embodiments, the DNA targeting composition, or the isolated polynucleotide sequence, or the vector, or the cell, or the pharmaceutical composition, or a combination thereof, is administered to skeletal muscle or cardiac muscle of the subject. In some embodiments, the disease comprises Duchenne muscular dystrophy (DMD) or Becker muscular dystrophy (BMD).

The disclosure provides for other aspects and embodiments that will be apparent in light of the following detailed description and accompanying figures.

### BRIEF DESCRIPTION OF THE DRAWINGS

FIG. 1 is an SDS-PAGE gel of the purified proteins Streptococcus uberis Cas9 (SuCas9, 138 kDa) and Streptococcus pyogenes Cas9 (SpCas9, 160 kDa).

FIG. 2 is a schematic diagram of the PAM sequence for SuCas9. The consensus PAM was determined to be NNAATA, with possible flexibility at positions 4 and 6 (G and C, respectively).

FIGS. 3A and 3B are graphs showing the indel frequency for SuCas9 for varying gRNA protospacer lengths for two gene targets, HBE1 (FIG. 3A) and TRAC (FIG. 3B), in mammalian cells.

FIG. 4 is a 1% agarose gel showing results from an in vitro cleavage assay for S. uberis Cas9 or S. pyogenes Cas9 protein. Successful SuCas9 cutting was expected to generate fragments of approximately 100 bp and 300 bp, while successful SpCas9 cutting was expected to generate fragments of approximately 200 bp and 190 bp.

FIG. 5 shows that S. uberis dCas9-KRAB mediates repression of a fluorescent HBE reporter. Flow cytometry of HBE repression in a transgenic K562 reporter cell line containing mCherry fluorescent protein sequence inserted at the 3′ end of the HBE gene. K562 HBE-mCherry cells were lentivirally transduced with either S. pyogenes dCas9-KRAB or S. uberis dCas9-KRAB (in a cassette containing a blasticidin resistance gene) and selected with blasticidin for 5 days to create a stable line. Then, Cas9-containing cells were lentivirally transduced with single gRNAs (in a cassette containing a puromycin resistance gene) and cultured for 10 days with puromycin selection on days 3-6. Cells were harvested and assayed for mCherry repression by flow cytometry. This is the raw data used to generate the bar plots in FIG. 16 for S. uberis.

FIG. 6 shows that S. uberis dCas9-KRAB mediates repression of HBE mRNA expression. To verify repression of HBE-mCherry at the transcript level with the novel DNA targeting system, RNA from cells harvested for flow cytometry in FIG. 5 as described above were used for qPCR with primers targeting HBE.

FIG. 7A is a graph showing relative HBG1 gene expression with S. uberis dCas9-p300, demonstrating activation of gene expression with the fusion protein. FIG. 7B is a graph showing relative IL1RN gene expression with S. uberis dCas9-p300, demonstrating activation of gene expression with the fusion protein.

FIG. 8A is the sequence logo produced for all sequences depleted a minimum of 10-fold in the empirical PAM determination assay for S. dysgalactiae Cas9. FIG. 8B is a table showing the percent of depleted sequences containing each nucleotide at each position for S. dysgalactiae Cas9. The allowed PAM sequence was found to be NNGGNTN for S. dysgalactiae Cas9, with a slight preference for C in the final position.

FIG. 9A is the sequence logo produced for all sequences depleted a minimum of 10-fold in the empirical PAM determination assay for S. gallolyticus Cas9. FIG. 9B is a table showing the percent of depleted sequences containing each nucleotide at each position for S. gallolyticus Cas9. The allowed PAM sequence for S. gallolyticus Cas9 was found to be NNG(T/C)(G/A)AN, with a slight preference for A in the final position.

FIG. 10A is the sequence logo produced for all sequences depleted a minimum of 10-fold in the empirical PAM determination assay for S. iniae Cas9. FIG. 10B is a table showing the percent of depleted sequences containing each nucleotide at each position for S. iniae Cas9. The allowed PAM sequence for S. iniae Cas9 was found to be NNGGNNN.

FIG. 11A is the sequence logo produced for all sequences depleted a minimum of 10-fold in the empirical PAM determination assay for S. lutetiensis Cas9. FIG. 11B is a table showing the percent of depleted sequences containing each nucleotide at each position for S. lutetiensis Cas9. The allowed PAM sequence for S. lutetiensis Cas9 was found to be NNAAAAN with a slight preference for A at the final position.

FIG. 12A is the sequence logo produced for all sequences depleted a minimum of 10-fold in the empirical PAM determination assay for S. parasanguinis Cas9. FIG. 12B is a table showing the percent of depleted sequences containing each nucleotide at each position for S. parasanguinis Cas9. The allowed PAM sequence for S. parasanguinis Cas9 was found to be NNAA(A/G)GN with a slight preference for G, C, or T at the final position.

FIG. 13A is the sequence logo produced for all sequences depleted a minimum of 10-fold in the empirical PAM determination assay for S. uberis Cas9. FIG. 13B is a table showing the percent of depleted sequences containing each nucleotide at each position for S. uberis Cas9. The allowed PAM sequence for S. uberis Cas9 was found to be NNA(A/G)TAN with a slight preference for G, C, or T at the final position.

FIGS. 14A-14B are graphs showing the level of repression of HBE-mCherry expression in K562 cells using dCas9-KRAB fusion proteins with a dCas9 protein from one of the various species. This graph shows the percent of HBE-mCherry low-expressing cells after transduction with a panel of dCas9-KRAB encoding lentiviruses and HBE-targeting sgRNA for each dCas9. Higher numbers indicate more repression. The dCas9 effectors that lead to at least double the level of downregulation as the Sp-dCas9 non-targeting control (Sp_NT) were considered as dCas9 sequences that are functional in mammalian cells. These were S. agalactiae, S. gallolyticus, S. iniae, S. lutetiensis, S. mutans, S. parauberis, S. parasanguinis and S. uberis.

FIGS. 15A-15B are graphs showing the level of repression of HBE-mCherry expression with fusion proteins including KRAB fused to a Cas9 protein from Streptococcus gallolyticus, Streptococcus iniae, Streptococcus parasanguinis, or Streptococcus uberis.

FIG. 16 is a graph showing the percentage of samples with an insertion or deletion, demonstrating nuclease activity of S. gallolyticus Cas9 and S. iniae Cas9 proteins in mammalian cells.

### DETAILED DESCRIPTION

Disclosed herein is a novel small Cas9 from a unique bacterial strain. The Cas9 may be from, for example, Streptococcus uberis, Streptococcus agalactiae, Streptococcus gallolyticus, Streptococcus iniae, Streptococcus lutetiensis, Streptococcus mutans, Streptococcus parauberis, Streptococcus dysgalactiae, or Streptococcus parasanguinis. Further disclosed herein is an RNA-guided DNA targeting system including the novel small Cas9 from a unique bacterial strain and associated gRNA sequences. The compositions and methods may include the 1122-amino acid Cas9 from Streptococcus uberis, for example, and at least one gRNA sequence. Further provided are repeat, tracrRNA, single guide RNA, and the protospacer adjacent motif (PAM) sequences. The Cas9 protein may include nuclease-inactivating mutations, resulting in DNA binding activity without cleavage (which may be referred to as null-nuclease, or dCas9). The compositions and methods disclosed herein may target any sequence in the set of mammalian genomes, provided it is upstream of the PAM. Null-nuclease novel Cas9 proteins such as S. uberis dCas9 may be fused to epigenetic modifier domain(s) to activate or repress target genes. A nuclease-competent version can be generated by reverting the inactivating mutations to wild-type, which may allow for the targeted cutting of mammalian genomes and genome editing. Further described herein are fusion proteins comprising the novel small Cas9.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Autologous” refers to any material derived from a subject and re-introduced to the same subject.

“Binding region” as used herein refers to the region within a target region that is recognized and bound by the CRISPR/Cas-based gene editing system.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, bladder, cervix, uterus, skin, epithelium, muscle, kidney, liver, lymph, bone, blood, ovary, prostate, lung, brain, head and neck, and/or breast. Cancer may include medullablastoma, non-small cell lung cancer, and/or mesothelioma. In embodiments detailed herein, the cancer includes leukemia. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the leukemia is chronic myeloid leukemia (CML). In some embodiments, the leukemia is acute myeloid leukemia (AML).

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated.

“Target region” as used herein refers to the region of the target gene to which the CRISPR/Cas9-based gene editing or targeting system is designed to bind.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. CRISPR/Cas-Based Gene Editing System

Provided herein are DNA Targeting Systems. A DNA Targeting System is a system capable of specifically targeting a particular region of DNA and modulating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

Each of these systems comprises a DNA-binding portion or domain, such as a guide RNA, a ZF, or a TALE, that specifically recognizes and binds to a particular target region of a target DNA. The DNA-binding portion (for example, Cas protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity, to form a fusion protein. Exemplary second polypeptide domains are detailed further below (see “Cas Fusion Protein”). For example, the DNA-binding portion can be linked to an activator and thus guide the activator to a specific target region of the target DNA. Similarly, the DNA-binding portion can be linked to a repressor and thus guide the repressor to a specific target region of the target DNA.

In some embodiments, the DNA-binding portion comprises a Cas protein, such as a Cas9 protein, and such systems are referred to as CRISPR/Cas9-based gene editing systems, or CRISPR/Cas-based gene editing systems. Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor. Some CRISPR-Cas-based systems can operate to activate or repress expression using the Cas protein linked to a second protein domain, such as, for example, an activator or repressor.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas9-based engineered systems for use in gene editing and treating genetic diseases. The CRISPR/Cas9-based engineered systems can be designed to target any gene, including genes involved in, for example, a genetic disease, aging, tissue regeneration, or wound healing. The CRISPR/Cas9-based gene editing system can include a Cas9 protein or a Cas9 fusion protein.

a. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. An example of a Cas9 molecule is a Streptococcus pyogenes Cas9 molecule (also referred herein as “SpCas9”). SpCas9 may comprise an amino acid sequence of SEQ ID NO: 26. Another example of a Cas9 molecule is a Staphylococcus aureus Cas9 molecule (also referred herein as “SaCas9”). SaCas9 may comprise an amino acid sequence of SEQ ID NO: 27.

Provided herein is a novel Cas9 protein. The novel Cas9 protein may be from, for example, Streptococcus uberis, Streptococcus agalactiae, Streptococcus gallolyticus, Streptococcus iniae, Streptococcus lutetiensis, Streptococcus mutans, Streptococcus parauberis, Streptococcus dysgalactiae, or Streptococcus parasanguinis. In some embodiments, the Cas9 protein is from Streptococcus uberis (SuCas9). SuCas9 may comprise an amino acid sequence of SEQ ID NO: 57, encoded by a polynucleotide comprising the sequence of SEQ ID NO: 58. SuCas9 may comprise an amino acid sequence having at least 80%, 85%, 90%, 95%, or 98% or greater identity to SEQ ID NO: 57, or any fragment thereof. SuCas9 may comprise an amino acid sequence having one, two, three, four, five or more changes selected from amino acid substitutions, insertions, or deletions, relative to SEQ ID NO: 57, or any fragment thereof. SuCas9 may be encoded by a polynucleotide comprising a sequence having at least 80%, 85%, 90%, 95%, or 98% or greater identity to SEQ ID NO: 58, or any fragment thereof. SuCas9 may be encoded by a polynucleotide comprising a sequence having one, two, three, four, five or more changes selected from nucleotide substitutions, insertions, or deletions, relative to SEQ ID NO: 58, or any fragment thereof.

In some embodiments, the Cas9 protein is from Streptococcus parasanguinis. S. parasanguinis Cas9 may comprise an amino acid sequence of SEQ ID NO: 223, encoded by a polynucleotide comprising the sequence of SEQ ID NO: 224. S. parasanguinis Cas9 may comprise an amino acid sequence having at least 80%, 85%, 90%, 95%, or 98% or greater identity to SEQ ID NO: 223, or any fragment thereof. S. parasanguinis Cas9 may comprise an amino acid sequence having one, two, three, four, five or more changes selected from amino acid substitutions, insertions, or deletions, relative to SEQ ID NO: 223, or any fragment thereof. S. parasanguinis Cas9 may be encoded by a polynucleotide comprising a sequence having at least 80%, 85%, 90%, 95%, or 98% or greater identity to SEQ ID NO: 224, or any fragment thereof. S. parasanguinis Cas9 may be encoded by a polynucleotide comprising a sequence having one, two, three, four, five or more changes selected from nucleotide substitutions, insertions, or deletions, relative to SEQ ID NO: 224, or any fragment thereof.

In some embodiments, the Cas9 protein is from Streptococcus agalactiae. S. agalactiae Cas9 may comprise an amino acid sequence of SEQ ID NO: 241, encoded by a polynucleotide comprising the sequence of SEQ ID NO: 242. S. agalactiae Cas9 may comprise an amino acid sequence having at least 80%, 85%, 90%, 95%, or 98% or greater identity to SEQ ID NO: 241, or any fragment thereof. S. agalactiae Cas9 may comprise an amino acid sequence having one, two, three, four, five or more changes selected from amino acid substitutions, insertions, or deletions, relative to SEQ ID NO: 241, or any fragment thereof. S. agalactiae Cas9 may be encoded by a polynucleotide comprising a sequence having at least 80%, 85%, 90%, 95%, or 98% or greater identity to SEQ ID NO: 242, or any fragment thereof. S. agalactiae Cas9 may be encoded by a polynucleotide comprising a sequence having one, two, three, four, five or more changes selected from nucleotide substitutions, insertions, or deletions, relative to SEQ ID NO: 242, or any fragment thereof.

In some embodiments, the Cas9 protein is from Streptococcus gallolyticus. S. gallolyticus Cas9 may comprise an amino acid sequence of SEQ ID NO: 243, encoded by a polynucleotide comprising the sequence of SEQ ID NO: 244. S. gallolyticus Cas9 may comprise an amino acid sequence having at least 80%, 85%, 90%, 95%, or 98% or greater identity to SEQ ID NO: 243, or any fragment thereof. S. gallolyticus Cas9 may comprise an amino acid sequence having one, two, three, four, five or more changes selected from amino acid substitutions, insertions, or deletions, relative to SEQ ID NO: 243, or any fragment thereof. S. gallolyticus Cas9 may be encoded by a polynucleotide comprising a sequence having at least 80%, 85%, 90%, 95%, or 98% or greater identity to SEQ ID NO: 244, or any fragment thereof. S. gallolyticus Cas9 may be encoded by a polynucleotide comprising a sequence having one, two, three, four, five or more changes selected from nucleotide substitutions, insertions, or deletions, relative to SEQ ID NO: 244, or any fragment thereof.

In some embodiments, the Cas9 protein is from Streptococcus iniae. S. iniae Cas9 may comprise an amino acid sequence of SEQ ID NO: 245, encoded by a polynucleotide comprising the sequence of SEQ ID NO: 246. S. iniae Cas9 may comprise an amino acid sequence having at least 80%, 85%, 90%, 95%, or 98% or greater identity to SEQ ID NO: 245, or any fragment thereof. S. iniae Cas9 may comprise an amino acid sequence having one, two, three, four, five or more changes selected from amino acid substitutions, insertions, or deletions, relative to SEQ ID NO: 245, or any fragment thereof. S. iniae Cas9 may be encoded by a polynucleotide comprising a sequence having at least 80%, 85%, 90%, 95%, or 98% or greater identity to SEQ ID NO: 246, or any fragment thereof. S. iniae Cas9 may be encoded by a polynucleotide comprising a sequence having one, two, three, four, five or more changes selected from nucleotide substitutions, insertions, or deletions, relative to SEQ ID NO: 246, or any fragment thereof.

In some embodiments, the Cas9 protein is from Streptococcus lutetiensis. S. lutetiensis Cas9 may comprise an amino acid sequence of SEQ ID NO: 247, encoded by a polynucleotide comprising the sequence of SEQ ID NO: 248. S. lutetiensis Cas9 may comprise an amino acid sequence having at least 80%, 85%, 90%, 95%, or 98% or greater identity to SEQ ID NO: 247, or any fragment thereof. S. lutetiensis Cas9 may comprise an amino acid sequence having one, two, three, four, five or more changes selected from amino acid substitutions, insertions, or deletions, relative to SEQ ID NO: 247, or any fragment thereof. S. lutetiensis Cas9 may be encoded by a polynucleotide comprising a sequence having at least 80%, 85%, 90%, 95%, or 98% or greater identity to SEQ ID NO: 248, or any fragment thereof. S. lutetiensis Cas9 may be encoded by a polynucleotide comprising a sequence having one, two, three, four, five or more changes selected from nucleotide substitutions, insertions, or deletions, relative to SEQ ID NO: 248, or any fragment thereof.

In some embodiments, the Cas9 protein is from Streptococcus mutans. S. mutans Cas9 may comprise an amino acid sequence of SEQ ID NO: 249, encoded by a polynucleotide comprising the sequence of SEQ ID NO: 250. S. mutans Cas9 may comprise an amino acid sequence having at least 80%, 85%, 90%, 95%, or 98% or greater identity to SEQ ID NO: 249, or any fragment thereof. S. mutans Cas9 may comprise an amino acid sequence having one, two, three, four, five or more changes selected from amino acid substitutions, insertions, or deletions, relative to SEQ ID NO: 249, or any fragment thereof. S. mutans Cas9 may be encoded by a polynucleotide comprising a sequence having at least 80%, 85%, 90%, 95%, or 98% or greater identity to SEQ ID NO: 250, or any fragment thereof. S. mutans Cas9 may be encoded by a polynucleotide comprising a sequence having one, two, three, four, five or more changes selected from nucleotide substitutions, insertions, or deletions, relative to SEQ ID NO: 250, or any fragment thereof.

In some embodiments, the Cas9 protein is from Streptococcus parauberis. S. parauberis Cas9 may comprise an amino acid sequence of SEQ ID NO: 251, encoded by a polynucleotide comprising the sequence of SEQ ID NO: 252. S. parauberis Cas9 may comprise an amino acid sequence having at least 80%, 85%, 90%, 95%, or 98% or greater identity to SEQ ID NO: 251, or any fragment thereof. S. parauberis Cas9 may comprise an amino acid sequence having one, two, three, four, five or more changes selected from amino acid substitutions, insertions, or deletions, relative to SEQ ID NO: 251, or any fragment thereof. S. parauberis Cas9 may be encoded by a polynucleotide comprising a sequence having at least 80%, 85%, 90%, 95%, or 98% or greater identity to SEQ ID NO: 252, or any fragment thereof. S. parauberis Cas9 may be encoded by a polynucleotide comprising a sequence having one, two, three, four, five or more changes selected from nucleotide substitutions, insertions, or deletions, relative to SEQ ID NO: 252, or any fragment thereof.

In some embodiments, the Cas9 protein is from Streptococcus dysgalactiae. S. dysgalactiae Cas9 may comprise an amino acid sequence of SEQ ID NO: 235, encoded by a polynucleotide comprising the sequence of SEQ ID NO: 236. S. dysgalactiae Cas9 may comprise an amino acid sequence having at least 80%, 85%, 90%, 95%, or 98% or greater identity to SEQ ID NO: 235, or any fragment thereof. S. dysgalactiae Cas9 may comprise an amino acid sequence having one, two, three, four, five or more changes selected from amino acid substitutions, insertions, or deletions, relative to SEQ ID NO: 235, or any fragment thereof. S. dysgalactiae Cas9 may be encoded by a polynucleotide comprising a sequence having at least 80%, 85%, 90%, 95%, or 98% or greater identity to SEQ ID NO: 236, or any fragment thereof. S. dysgalactiae Cas9 may be encoded by a polynucleotide comprising a sequence having one, two, three, four, five or more changes selected from nucleotide substitutions, insertions, or deletions, relative to SEQ ID NO: 236, or any fragment thereof.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi: 10.1038/nbt.2647). In certain embodiments, a Cas9 molecule of S. thermophilus recognizes the sequence motif NGGNG (SEQ ID NO: 4) and/or NNAGAAW (W=A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R=A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R=A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R=A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R=A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R=A or G; V=A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi: 10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R=A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R=A or G) (SEQ ID NO: 7), NNGRRN (R=A or G) (SEQ ID NO: 8), NNGRRT (R=A or G) (SEQ ID NO: 9), or NNGRRV (R=A or G; V=A or C or G) (SEQ ID NO: 10). In the aforementioned embodiments, N can be any nucleotide residue, for example, any of A, G, C, or T. In some embodiments, the Cas protein recognizes a PAM sequence of AATA (SEQ ID NO: 71), NNAATA (SEQ ID NO: 274), NNA(A/G)TAN (SEQ ID NO: 273), NNGTAAA (SEQ ID NO: 276), NNG(T/C)(G/A)AN (SEQ ID NO: 275), NNGGNNN (SEQ ID NO: 277), NGG (SEQ ID NO: 2), NNAAAAN (SEQ ID NO: 278), NNAAAAA (SEQ ID NO: 279), NNGGNTN (SEQ ID NO: 280), NNAA(A/G)GN (SEQ ID NO: 281), and/or NNAAAG (SEQ ID NO: 282). Streptococcus uberis Cas9 proteins as detailed herein may recognize a PAM polynucleotide comprising the sequence of AATA (SEQ ID NO: 71), NNA(A/G)TAN (SEQ ID NO: 273), and/or NNAATA (SEQ ID NO: 274). Streptococcus agalactiae Cas9 proteins as detailed herein may recognize a PAM polynucleotide comprising the sequence of NGG (SEQ ID NO: 2). Streptococcus gallolyticus Cas9 proteins as detailed herein may recognize a PAM polynucleotide comprising the sequence of NNG(T/C)(G/A)AN (SEQ ID NO: 275) and/or NNGTAAA (SEQ ID NO: 276). Streptococcus iniae Cas9 proteins as detailed herein may recognize a PAM polynucleotide comprising the sequence of NNGGNNN (SEQ ID NO: 277) and/or NGG (SEQ ID NO: 2). Streptococcus lutetiensis Cas9 proteins as detailed herein may recognize a PAM polynucleotide comprising the sequence of NNAAAAN (SEQ ID NO: 278) and/or NNAAAAA (SEQ ID NO: 279). Streptococcus mutans Cas9 proteins as detailed herein may recognize a PAM polynucleotide comprising the sequence of NGG (SEQ ID NO: 2). Streptococcus parauberis Cas9 proteins as detailed herein may recognize a PAM polynucleotide comprising the sequence of NGG (SEQ ID NO: 2). Streptococcus dysgalactiae Cas9 proteins as detailed herein may recognize a PAM polynucleotide comprising the sequence of NNGGNTN (SEQ ID NO: 280). Streptococcus parasanguinis Cas9 proteins as detailed herein may recognize a PAM polynucleotide comprising the sequence of NNAA(A/G)GN (SEQ ID NO: 281) and/or NNAAAG (SEQ ID NO: 282).

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 20).

In some embodiments, the at least one Cas9 molecule is a mutant Cas9 molecule. The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A, and/or D986A. A S. pyogenes Cas9 protein with the D10A mutation may comprise an amino acid sequence of SEQ ID NO: 28. A S. pyogenes Cas9 protein with D10A and H849A mutations may comprise an amino acid sequence of SEQ ID NO: 29. Exemplary mutations with reference to the S. aureus Cas9 sequence to inactivate the nuclease activity include D10A and N580A. In certain embodiments, the mutant S. aureus Cas9 molecule comprises a D10A mutation. The nucleotide sequence encoding this mutant S. aureus Cas9 is set forth in SEQ ID NO: 30. In certain embodiments, the mutant S. aureus Cas9 molecule comprises a N580A mutation. The nucleotide sequence encoding this mutant S. aureus Cas9 molecule is set forth in SEQ ID NO: 31.

Exemplary mutations with reference to the S. uberis Cas9 (SuCas9) sequence to inactivate the nuclease activity include D10A and/or H600A. In some embodiments, the SuCas9 comprises at least one amino acid mutation that knocks out nuclease activity of the Cas protein. In some embodiments, the SuCas9 protein includes at least one amino acid mutation selected from at least one of D10A and H600A. Su-dCas9 may comprise the amino acid sequence of SEQ ID NO: 59, encoded by a polynucleotide comprising the sequence of SEQ ID NO: 60. Su-dCas9 may comprise an amino acid sequence having at least 80%, 85%, 90%, 95%, or 98% or greater identity to SEQ ID NO: 59, or any fragment thereof. Su-dCas9 may comprise an amino acid sequence having one, two, three, four, five or more changes selected from amino acid substitutions, insertions, or deletions, relative to SEQ ID NO: 59, or any fragment thereof. Su-dCas9 may be encoded by a polynucleotide comprising a sequence having at least 80%, 85%, 90%, 95%, or 98% or greater identity to SEQ ID NO: 60, or any fragment thereof. Su-dCas9 may be encoded by a polynucleotide comprising a sequence having one, two, three, four, five or more changes selected from nucleotide substitutions, insertions, or deletions, relative to SEQ ID NO: 60, or any fragment thereof.

Exemplary mutations with reference to the Streptococcus agalactiae Cas9 sequence to inactivate the nuclease activity include D10A and/or H845A. In some embodiments, the Streptococcus agalactiae Cas9 comprises at least one amino acid mutation that knocks out nuclease activity of the Cas protein. In some embodiments, the Streptococcus agalactiae Cas9 protein includes at least one amino acid mutation selected from D10A and H845A. Streptococcus agalactiae dCas9 may comprise the amino acid sequence of SEQ ID NO: 193, encoded by a polynucleotide comprising the sequence of SEQ ID NO: 194. Streptococcus agalactiae dCas9 may comprise an amino acid sequence having at least 80%, 85%, 90%, 95%, or 98% or greater identity to SEQ ID NO: 193, or any fragment thereof. Streptococcus agalactiae dCas9 may comprise an amino acid sequence having one, two, three, four, five or more changes selected from amino acid substitutions, insertions, or deletions, relative to SEQ ID NO: 193, or any fragment thereof. Streptococcus agalactiae dCas9 may be encoded by a polynucleotide comprising a sequence having at least 80%, 85%, 90%, 95%, or 98% or greater identity to SEQ ID NO: 194, or any fragment thereof. Streptococcus agalactiae dCas9 may be encoded by a polynucleotide comprising a sequence having one, two, three, four, five or more changes selected from nucleotide substitutions, insertions, or deletions, relative to SEQ ID NO: 194, or any fragment thereof.

Exemplary mutations with reference to the Streptococcus gallolyticus Cas9 sequence to inactivate the nuclease activity include D10A and/or H599A. In some embodiments, the Streptococcus gallolyticus Cas9 comprises at least one amino acid mutation that knocks out nuclease activity of the Cas protein. In some embodiments, the Streptococcus gallolyticus Cas9 protein includes at least one amino acid mutation selected from D10A and H599A. Streptococcus gallolyticus dCas9 may comprise the amino acid sequence of SEQ ID NO: 197, encoded by a polynucleotide comprising the sequence of SEQ ID NO: 198. Streptococcus gallolyticus dCas9 may comprise an amino acid sequence having at least 80%, 85%, 90%, 95%, or 98% or greater identity to SEQ ID NO: 197, or any fragment thereof. Streptococcus gallolyticus dCas9 may comprise an amino acid sequence having one, two, three, four, five or more changes selected from amino acid substitutions, insertions, or deletions, relative to SEQ ID NO: 197, or any fragment thereof. Streptococcus gallolyticus dCas9 may be encoded by a polynucleotide comprising a sequence having at least 80%, 85%, 90%, 95%, or 98% or greater identity to SEQ ID NO: 198, or any fragment thereof. Streptococcus gallolyticus dCas9 may be encoded by a polynucleotide comprising a sequence having one, two, three, four, five or more changes selected from nucleotide substitutions, insertions, or deletions, relative to SEQ ID NO: 198, or any fragment thereof.

Exemplary mutations with reference to the Streptococcus iniae Cas9 sequence to inactivate the nuclease activity include D10A and/or H840A. In some embodiments, the Streptococcus iniae Cas9 comprises at least one amino acid mutation that knocks out nuclease activity of the Cas protein. In some embodiments, the Streptococcus iniae Cas9 protein includes at least one amino acid mutation selected from D10A and H840A. Streptococcus iniae dCas9 may comprise the amino acid sequence of SEQ ID NO: 201, encoded by a polynucleotide comprising the sequence of SEQ ID NO: 202. Streptococcus iniae dCas9 may comprise an amino acid sequence having at least 80%, 85%, 90%, 95%, or 98% or greater identity to SEQ ID NO: 201, or any fragment thereof. Streptococcus iniae dCas9 may comprise an amino acid sequence having one, two, three, four, five or more changes selected from amino acid substitutions, insertions, or deletions, relative to SEQ ID NO: 201, or any fragment thereof. Streptococcus iniae dCas9 may be encoded by a polynucleotide comprising a sequence having at least 80%, 85%, 90%, 95%, or 98% or greater identity to SEQ ID NO: 202, or any fragment thereof. Streptococcus iniae dCas9 may be encoded by a polynucleotide comprising a sequence having one, two, three, four, five or more changes selected from nucleotide substitutions, insertions, or deletions, relative to SEQ ID NO: 202, or any fragment thereof.

Exemplary mutations with reference to the Streptococcus lutetiensis Cas9 sequence to inactivate the nuclease activity include D10A and/or H599A. In some embodiments, the Streptococcus lutetiensis Cas9 comprises at least one amino acid mutation that knocks out nuclease activity of the Cas protein. In some embodiments, the Streptococcus lutetiensis Cas9 protein includes at least one amino acid mutation selected from D10A and H599A. Streptococcus lutetiensis dCas9 may comprise the amino acid sequence of SEQ ID NO: 205, encoded by a polynucleotide comprising the sequence of SEQ ID NO: 206. Streptococcus lutetiensis dCas9 may comprise an amino acid sequence having at least 80%, 85%, 90%, 95%, or 98% or greater identity to SEQ ID NO: 205, or any fragment thereof. Streptococcus lutetiensis dCas9 may comprise an amino acid sequence having one, two, three, four, five or more changes selected from amino acid substitutions, insertions, or deletions, relative to SEQ ID NO: 205, or any fragment thereof. Streptococcus lutetiensis dCas9 may be encoded by a polynucleotide comprising a sequence having at least 80%, 85%, 90%, 95%, or 98% or greater identity to SEQ ID NO: 206, or any fragment thereof. Streptococcus lutetiensis dCas9 may be encoded by a polynucleotide comprising a sequence having one, two, three, four, five or more changes selected from nucleotide substitutions, insertions, or deletions, relative to SEQ ID NO: 206, or any fragment thereof.

Exemplary mutations with reference to the Streptococcus mutans Cas9 sequence to inactivate the nuclease activity include D10A and/or H840A. In some embodiments, the Streptococcus mutans Cas9 comprises at least one amino acid mutation that knocks out nuclease activity of the Cas protein. In some embodiments, the Streptococcus mutans Cas9 protein includes at least one amino acid mutation selected from D10A and H840A. Streptococcus mutans dCas9 may comprise the amino acid sequence of SEQ ID NO: 209, encoded by a polynucleotide comprising the sequence of SEQ ID NO: 210. Streptococcus mutans dCas9 may comprise an amino acid sequence having at least 80%, 85%, 90%, 95%, or 98% or greater identity to SEQ ID NO: 209, or any fragment thereof. Streptococcus mutans dCas9 may comprise an amino acid sequence having one, two, three, four, five or more changes selected from amino acid substitutions, insertions, or deletions, relative to SEQ ID NO: 209, or any fragment thereof. Streptococcus mutans dCas9 may be encoded by a polynucleotide comprising a sequence having at least 80%, 85%, 90%, 95%, or 98% or greater identity to SEQ ID NO: 210, or any fragment thereof. Streptococcus mutans dCas9 may be encoded by a polynucleotide comprising a sequence having one, two, three, four, five or more changes selected from nucleotide substitutions, insertions, or deletions, relative to SEQ ID NO: 210, or any fragment thereof.

Exemplary mutations with reference to the Streptococcus parauberis Cas9 sequence to inactivate the nuclease activity include D10A and/or H840A. In some embodiments, the Streptococcus parauberis Cas9 comprises at least one amino acid mutation that knocks out nuclease activity of the Cas protein. In some embodiments, the Streptococcus parauberis Cas9 protein includes at least one amino acid mutation selected from D10A and H840A. Streptococcus parauberis dCas9 may comprise the amino acid sequence of SEQ ID NO: 213, encoded by a polynucleotide comprising the sequence of SEQ ID NO: 214. Streptococcus parauberis dCas9 may comprise an amino acid sequence having at least 80%, 85%, 90%, 95%, or 98% or greater identity to SEQ ID NO: 213, or any fragment thereof. Streptococcus parauberis dCas9 may comprise an amino acid sequence having one, two, three, four, five or more changes selected from amino acid substitutions, insertions, or deletions, relative to SEQ ID NO: 213, or any fragment thereof. Streptococcus parauberis dCas9 may be encoded by a polynucleotide comprising a sequence having at least 80%, 85%, 90%, 95%, or 98% or greater identity to SEQ ID NO: 214, or any fragment thereof. Streptococcus parauberis dCas9 may be encoded by a polynucleotide comprising a sequence having one, two, three, four, five or more changes selected from nucleotide substitutions, insertions, or deletions, relative to SEQ ID NO: 214 or any fragment thereof.

Exemplary mutations with reference to the Streptococcus parasanguinis Cas9 sequence to inactivate the nuclease activity include D9A and/or H604A. In some embodiments, the Streptococcus parasanguinis Cas9 comprises at least one amino acid mutation that knocks out nuclease activity of the Cas protein. In some embodiments, the Streptococcus parasanguinis Cas9 protein includes at least one amino acid mutation selected from D9A and H604A. Streptococcus parasanguinis dCas9 may comprise the amino acid sequence of SEQ ID NO: 225, encoded by a polynucleotide comprising the sequence of SEQ ID NO: 226. Streptococcus parasanguinis dCas9 may comprise an amino acid sequence having at least 80%, 85%, 90%, 95%, or 98% or greater identity to SEQ ID NO: 225, or any fragment thereof. Streptococcus parasanguinis dCas9 may comprise an amino acid sequence having one, two, three, four, five or more changes selected from amino acid substitutions, insertions, or deletions, relative to SEQ ID NO: 225, or any fragment thereof. Streptococcus parasanguinis dCas9 may be encoded by a polynucleotide comprising a sequence having at least 80%, 85%, 90%, 95%, or 98% or greater identity to SEQ ID NO: 226, or any fragment thereof. Streptococcus parasanguinis dCas9 may be encoded by a polynucleotide comprising a sequence having one, two, three, four, five or more changes selected from nucleotide substitutions, insertions, or deletions, relative to SEQ ID NO: 226 or any fragment thereof.

Exemplary mutations with reference to the Streptococcus dysgalactiae Cas9 sequence to inactivate the nuclease activity include D10A and H839A. In some embodiments, the Streptococcus dysgalactiae Cas9 comprises at least one amino acid mutation that knocks out nuclease activity of the Cas protein. In some embodiments, the Streptococcus dysgalactiae Cas9 protein includes at least one amino acid mutation selected from D10A and H839A. Streptococcus dysgalactiae dCas9 may comprise the amino acid sequence of SEQ ID NO: 237, encoded by a polynucleotide comprising the sequence of SEQ ID NO: 238. Streptococcus dysgalactiae dCas9 may comprise an amino acid sequence having at least 80%, 85%, 90%, 95%, or 98% or greater identity to SEQ ID NO: 237, or any fragment thereof. Streptococcus dysgalactiae dCas9 may comprise an amino acid sequence having one, two, three, four, five or more changes selected from amino acid substitutions, insertions, or deletions, relative to SEQ ID NO: 237, or any fragment thereof. Streptococcus dysgalactiae dCas9 may be encoded by a polynucleotide comprising a sequence having at least 80%, 85%, 90%, 95%, or 98% or greater identity to SEQ ID NO: 238, or any fragment thereof. Streptococcus dysgalactiae dCas9 may be encoded by a polynucleotide comprising a sequence having one, two, three, four, five or more changes selected from nucleotide substitutions, insertions, or deletions, relative to SEQ ID NO: 238 or any fragment thereof. Exemplary Cas9 proteins and exemplary associated sequences are shown in TABLE 8.

TABLE 8
Various Cas9 proteins and exemplary associated sequences.

| | | gRNA | | | dCas9- | dCas9- |
|---|---|---|---|---|---|---|
| Species | PAM | scaffold | Cas9 | dCas9 | KRAB | p300 |
| Streptococcus | NRG (SEQ ID | SEQ ID | SEQ ID | SEQ ID | SEQ ID | SEQ ID |
| pyogenes | NO:1) | NO: 19 | NO: 26 | NOs: | NO: 47 | NO: 255 |
| | | (RNA); | (protein) | 28, 29 | (protein); | (protein); |
| | | SEQ ID | | (protein) | SEQ ID | SEQ ID |
| | | NO: 18 | | | NO: 48 | NO: 256 |
| | | (DNA) | | | (DNA) | (DNA) |
| Staphylococcus | NNGRR (SEQ ID | SEQ ID | SEQ ID | SEQ ID | SEQ ID | SEQ ID |
| aureus | NO: 7) | NO: 19 | NO: 27 | NOs: | NO: 49 | NO: 257 |
| | | (RNA); | (protein) | 30, 31 | (protein); | (protein); |
| | | SEQ ID | | (DNA) | SEQ ID | SEQ ID |
| | | NO: 18 | | | NO: 50 | NO: 258 |
| | | (DNA) | | | (DNA) | (DNA) |
| Streptococcus | NNA(A/G)TAN | SEQ ID | SEQ ID | SEQ ID | SEQ ID | SEQ ID |
| uberis | with slight | NO: 69 | NO: 57 | NO: 59 | NO: 61 | NO: 253 |
| | preference for G, | (RNA); | (protein); | (protein); | (protein); | (protein); |
| | C, or T in final | SEQ ID | SEQ ID | SEQ ID | SEQ ID | SEQ ID |
| | position (SEQ ID | NO: 70 | NO: 58 | NO: 60 | NO: 62 | NO: 254 |
| | NO: 273); | (DNA) | (DNA) | (DNA) | (DNA) | (DNA) |
| | NNAATA (SEQ | | | | | |
| | ID NO: 274); | | | | | |
| | AATA (SEQ ID | | | | | |
| | NO: 71) | | | | | |
| Streptococcus | NGG (SEQ ID | SEQ ID | SEQ ID | SEQ ID | SEQ ID | SEQ ID |
| agalactiae | NO: 2) | NO: | NO: 241 | NO: 193 | NO: 217 | NO: 259 |
| | | 195 | (protein); | (protein); | (protein) | (protein); |
| | | (RNA); | SEQ ID | SEQ ID | | SEQ ID |
| | | SEQ ID | NO: 242 | NO: 194 | | NO: 260 |
| | | NO: | (DNA) | (DNA) | | (DNA) |
| | | 196 | | | | |
| | | (DNA) | | | | |
| Streptococcus | NNG(T/C)(G/A)AN | SEQ ID | SEQ ID | SEQ ID | SEQ ID | SEQ ID |
| gallolyticus | with slight | NO: | NO: 243 | NO: 197 | NO: 218 | NO: 263 |
| | preference for A | 199 | (protein); | (protein); | (protein) | (protein); |
| | in final position | (RNA); | SEQ ID | SEQ ID | | SEQ ID |
| | (SEQ ID NO: | SEQ ID | NO: 244 | NO: 198 | | NO: 264 |
| | 275); | NO: | (DNA) | (DNA) | | (DNA) |
| | NNGTAAA (SEQ | 200 | | | | |
| | ID NO: 276) | (DNA) | | | | |
| Streptococcus | NNGGNNN (SEQ | SEQ ID | SEQ ID | SEQ ID | SEQ ID | SEQ ID |
| iniae | ID NO: 277); | NO: | NO: 245 | NO: 201 | NO: 219 | NO: 265 |
| | NGG (SEQ ID | 203 | (protein); | (protein); | (protein) | (protein); |
| | NO: 2) | (RNA); | SEQ ID | SEQ ID | | SEQ ID |
| | | SEQ ID | NO: 246 | NO: 202 | | NO: 266 |
| | | NO: | (DNA) | (DNA) | | (DNA) |
| | | 204 | | | | |
| | | (DNA) | | | | |
| Streptococcus | NNAAAAN | SEQ ID | SEQ ID | SEQ ID | SEQ ID | SEQ ID |
| lutetiensis | with slight | NO: | NO: 247 | NO: 205 | NO: 220 | NO: 267 |
| | preference for A | 207 | (protein); | (protein); | (protein) | (protein); |
| | in final position | (RNA); | SEQ ID | SEQ ID | | SEQ ID |
| | (SEQ ID NO: | SEQ ID | NO: 248 | NO: 206 | | NO: 268 |
| | 278); | NO: | (DNA) | (DNA) | | (DNA) |
| | NNAAAAA (SEQ | 208 | | | | |
| | ID NO: 279) | (DNA) | | | | |
| Streptococcus | NGG (SEQ ID | SEQ ID | SEQ ID | SEQ ID | SEQ ID | SEQ ID |
| mutans | NO: 2) | NO: | NO: 249 | NO: 209 | NO: 221 | NO: 261 |
| | | 211 | (protein); | (protein); | (protein) | (protein); |
| | | (RNA); | SEQ ID | SEQ ID | | SEQ ID |
| | | SEQ ID | NO: 250 | NO: 210 | | NO: 262 |
| | | NO: | (DNA) | (DNA) | | (DNA) |
| | | 212 | | | | |
| | | (DNA) | | | | |
| Streptococcus | NGG (SEQ ID | SEQ ID | SEQ ID | SEQ ID | SEQ ID | SEQ ID |
| parauberis | NO: 2) | NO: | NO: 251 | NO: 213 | NO: 222 | NO: 269 |
| | | 215 | (protein); | (protein); | (protein) | (protein); |
| | | (RNA); | SEQ ID | SEQ ID | | SEQ ID |
| | | SEQ ID | NO: 252 | NO: 214 | | NO: 270 |
| | | NO: | (DNA) | (DNA) | | (DNA) |
| | | 216 | | | | |
| | | (DNA) | | | | |
| Streptococcus | NNGGNTN | SEQ ID | SEQ ID | SEQ ID | SEQ ID | SEQ ID |
| dysgalactiae | with slight | NO: | NO: 235 | NO: 237 | NO: 239 | NO: 271 |
| | preference for C | 233 | (protein); | (protein); | (protein); | (protein); |
| | in final position | (RNA); | SEQ ID | SEQ ID | SEQ ID | SEQ ID |
| | (SEQ ID NO: 280) | SEQ ID | NO: 236 | NO: 238 | NO: 240 | NO: 272 |
| | | NO: | (DNA) | (DNA) | (DNA) | (DNA) |
| | | 234 | | | | |
| | | (DNA) | | | | |
| Streptococcus | NNAA(A/G)GN | SEQ ID | SEQ ID | SEQ ID | SEQ ID | SEQ ID |
| parasanguinis | with slight | NO: | NO: 223 | NO: 225 | NO: 227 | NO: 229 |
| | preference for G, | 231 | (protein); | (protein); | (protein); | (protein); |
| | C, or T in final | (RNA); | SEQ ID | SEQ ID | SEQ ID | SEQ ID |
| | position (SEQ ID | SEQ ID | NO: 224 | NO: 226 | NO: 228 | NO: 230 |
| | NO: 281); | NO: | (DNA) | (DNA) | (DNA) | (DNA) |
| | NNAAAG (SEQ | 232 | | | | |
| | ID NO: 282) | (DNA) | | | | |

In some embodiments, the Cas9 protein further includes a purification tag, such as a His tag. SpCas9 with a His tag may comprise an amino acid sequence of SEQ ID NO: 64. SuCas9 with a His tag may comprise an amino acid sequence of SEQ ID NO: 63.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 32. Exemplary codon optimized nucleic acid sequences encoding a Cas9 molecule of S. aureus, and optionally containing nuclear localization sequences (NLSs), are set forth in SEQ ID NOs: 33-39. Another exemplary codon optimized nucleic acid sequence encoding a Cas9 molecule of S. aureus comprises the nucleotides 1293-4451 of SEQ ID NO: 40.

b. Cas Fusion Protein

Alternatively or additionally, the CRISPR/Cas-based gene editing system can include a fusion protein. The fusion protein can comprise two heterologous polypeptide domains. The first polypeptide domain comprises a Cas protein or a mutated Cas protein. The first polypeptide domain is fused to at least one second polypeptide domain. The second polypeptide domain has a different activity that what is endogenous to Cas protein. For example, the second polypeptide domain may have an activity such as transcription activation activity, transcription repression activity, transcription release factor activity, histone modification activity, nuclease activity, nucleic acid association activity, histone methylase activity, DNA methylase activity, histone demethylase activity, DNA demethylase activity, acetylation activity, and/or deacetylation activity. The activity of the second polypeptide domain may be direct or indirect. The second polypeptide domain may have this activity itself (direct), or it may recruit and/or interact with a polypeptide domain that has this activity (indirect). In some embodiments, the second polypeptide domain has transcription activation activity. In some embodiments, the second polypeptide domain has transcription repression activity. In some embodiments, the second polypeptide domain comprises a synthetic transcription factor. The second polypeptide domain may be at the C-terminal end of the first polypeptide domain, or at the N-terminal end of the first polypeptide domain, or a combination thereof. The fusion protein may include one second polypeptide domain. The fusion protein may include two of the second polypeptide domains. For example, the fusion protein may include a second polypeptide domain at the N-terminal end of the first polypeptide domain as well as a second polypeptide domain at the C-terminal end of the first polypeptide domain. In other embodiments, the fusion protein may include a single first polypeptide domain and more than one (for example, two or three) second polypeptide domains in tandem.

The linkage from the first polypeptide domain to the second polypeptide domain can be through reversible or irreversible covalent linkage or through a non-covalent linkage, as long as the linker does not interfere with the function of the second polypeptide domain. For example, a Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

In some embodiments, the fusion protein includes at least one linker. A linker may be included anywhere in the polypeptide sequence of the fusion protein, for example, between the first and second polypeptide domains. A linker may be of any length and design to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may include, for example, a GS linker (Gly-Gly-Gly-Gly-Ser) n, wherein n is an integer between 0 and 10 (SEQ ID NO: 21). In a GS linker, n can be adjusted to optimize the linker length and achieve appropriate separation of the functional domains. Other examples of linkers may include, for example, Gly-Gly-Gly-Gly-Gly (SEQ ID NO: 22), Gly-Gly-Ala-Gly-Gly (SEQ ID NO: 23), Gly/Ser rich linkers such as Gly-Gly-Gly-Gly-Ser-Ser-Ser (SEQ ID NO: 24), or Gly/Ala rich linkers such as Gly-Gly-Gly-Gly-Ala-Ala-Ala (SEQ ID NO: 25).

In some embodiments, the Cas protein and/or the Cas fusion protein and/or gRNAs detailed herein may be used in compositions and methods for modulating expression of gene. Modulating may include, for example, increasing or enhancing expression of the gene, or reducing or inhibiting expression of the gene. The expression of the gene may be modulated by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be modulated by about 5-95%, 10-90%, 15-85%, 20-80%, or 1.5-fold to 10-fold, relative to a control. The expression of the gene may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be reduced by about 5-95%, 10-90%, 15-85%, 20-80%, or 1.5-fold to 10-fold, relative to a control. The expression of the gene may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control.

### i) Transcription Activation Activity

The second polypeptide domain can have transcription activation activity, for example, a transactivation domain. For example, gene expression of endogenous mammalian genes, such as human genes, can be achieved by targeting a fusion protein of a first polypeptide domain, such as dCas9, and a transactivation domain to mammalian promoters via combinations of gRNAs. The transactivation domain can include a VP16 protein, multiple VP16 proteins, such as a VP48 domain or VP64 domain, p65 domain of NF kappa B transcription activator activity, TET1, VPR, VPH, Rta, and/or p300. For example, the fusion protein may comprise dCas9-p300. In some embodiments, p300 comprises a polypeptide having the amino acid sequence of SEQ ID NO: 41 or SEQ ID NO: 42. The fusion protein may comprise Streptococcus pyogenes dCas9-p300 (protein sequence comprising SEQ ID NO: 255, polynucleotide sequence comprising SEQ ID NO: 256). The fusion protein may comprise Staphylococcus aureus dCas9-p300 (protein sequence comprising SEQ ID NO: 257, polynucleotide sequence comprising SEQ ID NO: 258). The fusion protein may comprise Streptococcus parasanguinis dCas9-p300 (protein sequence comprising SEQ ID NO: 229, polynucleotide sequence comprising SEQ ID NO: 230). The fusion protein may comprise Streptococcus uberis dCas9-p300 (protein sequence comprising SEQ ID NO: 253, polynucleotide sequence comprising SEQ ID NO: 254). The fusion protein may comprise Streptococcus agalactiae dCas9-p300 (protein sequence comprising SEQ ID NO: 259, polynucleotide sequence comprising SEQ ID NO: 260). The fusion protein may comprise Streptococcus gallolyticus dCas9-p300 (protein sequence comprising SEQ ID NO: 263, polynucleotide sequence comprising SEQ ID NO: 264). The fusion protein may comprise Streptococcus iniae dCas9-p300 (protein sequence comprising SEQ ID NO: 265, polynucleotide sequence comprising SEQ ID NO: 266). The fusion protein may comprise Streptococcus lutetiensis dCas9-p300 (protein sequence comprising SEQ ID NO: 267, polynucleotide sequence comprising SEQ ID NO: 268). The fusion protein may comprise Streptococcus mutans dCas9-p300 (protein sequence comprising SEQ ID NO: 261, polynucleotide sequence comprising SEQ ID NO: 262). The fusion protein may comprise Streptococcus parauberis dCas9-p300 (protein sequence comprising SEQ ID NO: 269, polynucleotide sequence comprising SEQ ID NO: 270). The fusion protein may comprise Streptococcus dysgalactiae dCas9-p300 (protein sequence comprising SEQ ID NO: 271, polynucleotide sequence comprising SEQ ID NO: 272). In other embodiments, the fusion protein comprises dCas9-VP64. In other embodiments, the fusion protein comprises VP64-dCas9-VP64. VP64-dCas9-VP64 may comprise a polypeptide having the amino acid sequence of SEQ ID NO: 43, encoded by a polynucleotide comprising the sequence of SEQ ID NO: 44. VPH may comprise a polypeptide having the amino acid sequence of SEQ ID NO: 53, encoded by a polynucleotide comprising the sequence of SEQ ID NO: 54. VPR may comprise a polypeptide having the amino acid sequence of SEQ ID NO: 55, encoded by a polynucleotide comprising the sequence of SEQ ID NO: 56.

### ii) Transcription Repression Activity

The second polypeptide domain can have transcription repression activity. Non-limiting examples of repressors include Kruppel associated box activity such as a KRAB domain or KRAB, MECP2, EED, ERF repressor domain (ERD), Mad mSIN3 interaction domain (SID) or Mad-SID repressor domain, SID4X repressor domain, Mxil repressor domain, SUV39H1, SUV39H2, G9A, ESET/SETBD1, Cir4, Su (var) 3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, and/or a domain having TATA box binding protein activity, or a combination thereof. In some embodiments, the second polypeptide domain has a KRAB domain activity, ERF repressor domain activity, Mxil repressor domain activity, SID4X repressor domain activity, Mad-SID repressor domain activity, DNMT3A or DNMT3L or fusion thereof activity, LSD1 histone demethylase activity, or TATA box binding protein activity. In some embodiments, the polypeptide domain comprises KRAB. KRAB may comprise a polypeptide having the amino acid sequence of SEQ ID NO: 45, encoded by a polynucleotide comprising the sequence of SEQ ID NO: 46. For example, the fusion protein may be S. pyogenes dCas9-KRAB (protein sequence comprising SEQ ID NO: 47; polynucleotide sequence comprising SEQ ID NO: 48). The fusion protein may comprise S. aureus dCas9-KRAB (protein sequence comprising SEQ ID NO: 49; polynucleotide sequence comprising SEQ ID NO: 50). The fusion protein may comprise S. pyogenes dCas9-KRAB (protein sequence comprising SEQ ID NO: 47; polynucleotide sequence comprising SEQ ID NO: 48). The fusion protein may comprise S. uberis dCas9-KRAB (protein sequence comprising SEQ ID NO: 61; polynucleotide sequence comprising SEQ ID NO: 62). The fusion protein may comprise Streptococcus agalactiae dCas9-KRAB (protein sequence comprising SEQ ID NO: 217). The fusion protein may comprise Streptococcus gallolyticus dCas9-KRAB (protein sequence comprising SEQ ID NO: 218). The fusion protein may comprise Streptococcus iniae dCas9-KRAB (protein sequence comprising SEQ ID NO: 219). The fusion protein may comprise Streptococcus lutetiensis dCas9-KRAB (protein sequence comprising SEQ ID NO: 220). The fusion protein may comprise Streptococcus mutans dCas9-KRAB (protein sequence comprising SEQ ID NO: 221). The fusion protein may comprise Streptococcus parauberis dCas9-KRAB (protein sequence comprising SEQ ID NO: 222). The fusion protein may comprise Streptococcus dysgalactiae dCas9-KRAB (protein sequence comprising SEQ ID NO: 239, polynucleotide sequence comprising SEQ ID NO: 240). The fusion protein may comprise Streptococcus parasanguinis dCas9-KRAB (protein sequence comprising SEQ ID NO: 227, polynucleotide sequence comprising SEQ ID NO: 228).

iii) Transcription Release Factor Activity

The second polypeptide domain can have transcription release factor activity. The second polypeptide domain can have eukaryotic release factor 1 (ERF1) activity or eukaryotic release factor 3 (ERF3) activity.

### iv) Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof. For example, the fusion protein may be dCas9-p300. In some embodiments, p300 comprises a polypeptide of SEQ ID NO: 41 or SEQ ID NO: 42.

### v) Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

### vi) Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, and TAL effector DNA-binding domain.

vii) Methylase Activity

The second polypeptide domain can have methylase activity, which involves transferring a methyl group to DNA, RNA, protein, small molecule, cytosine, or adenine. In some embodiments, the second polypeptide domain includes a DNA methyltransferase.

viii) Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; amino acid sequence comprising SEQ ID NO: 51; polynucleotide sequence comprising SEQ ID NO: 52). In some embodiments, the second polypeptide domain has histone demethylase activity. In some embodiments, the second polypeptide domain has DNA demethylase activity.

c. Guide RNA (gRNA)

The CRISPR/Cas-based gene editing system includes at least one gRNA molecule. For example, the CRISPR/Cas-based gene editing system may include two gRNA molecules. The at least one gRNA molecule can bind and recognize a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

For the S. uberis Cas9 proteins detailed herein, the gRNA may comprise the sequence of SEQ ID NO: 65, encoded by a sequence comprising SEQ ID NO: 66. The gRNA may comprise a tracrRNA comprising the sequence of SEQ ID NO: 67, encoded by a sequence comprising SEQ ID NO: 68. The gRNA may comprise a constant region, the constant region comprising the sequence of SEQ ID NO: 69, encoded by a sequence comprising SEQ ID NO: 70.

For Streptococcus agalactiae Cas9 proteins detailed herein, the gRNA or gRNA scaffold may comprise the sequence of SEQ ID NO: 195, encoded by a sequence comprising SEQ ID NO: 196. For Streptococcus gallolyticus Cas9 proteins detailed herein, the gRNA or gRNA scaffold may comprise the sequence of SEQ ID NO: 199, encoded by a sequence comprising SEQ ID NO: 200. For Streptococcus iniae Cas9 proteins detailed herein, the gRNA or gRNA scaffold may comprise the sequence of SEQ ID NO: 203, encoded by a sequence comprising SEQ ID NO: 204. For Streptococcus lutetiensis Cas9 proteins detailed herein, the gRNA or gRNA scaffold may comprise the sequence of SEQ ID NO: 207, encoded by a sequence comprising SEQ ID NO: 208 For Streptococcus mutans Cas9 proteins detailed herein, the gRNA or gRNA scaffold may comprise the sequence of SEQ ID NO: 211, encoded by a sequence comprising SEQ ID NO: 212. For Streptococcus parauberis Cas9 proteins detailed herein, the gRNA or gRNA scaffold may comprise the sequence of SEQ ID NO: 215, encoded by a sequence comprising SEQ ID NO: 216. For Streptococcus dysgalactiae Cas9 proteins detailed herein, the gRNA or gRNA scaffold may comprise the sequence of SEQ ID NO: 233, encoded by a sequence comprising SEQ ID NO: 234. For Streptococcus parasanguinis Cas9 proteins detailed herein, the gRNA or gRNA scaffold may comprise the sequence of SEQ ID NO: 231, encoded by a sequence comprising SEQ ID NO: 232.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target the Cas9 protein or fusion protein to a gene or a regulatory element thereof. The gRNA may target the Cas protein or fusion protein to a non-open chromatin region, an open chromatin region, a transcribed region of the target gene, a region upstream of a transcription start site of the target gene, a regulatory element of the target gene, an intron of the target gene, or an exon of the target gene, or a combination thereof. In some embodiments, the gRNA targets the Cas9 protein or fusion protein to a promoter of a gene. In some embodiments, the target region is located between about 1 to about 1000 base pairs upstream of a transcription start site of a target gene. In some embodiments, the DNA targeting composition comprises two or more gRNAs, each gRNA binding to a different target region.

The gRNA may target a region within/near the HBE gene. The gRNA may target a region within/near the TRAC gene. The gRNA may comprise a polynucleotide sequence comprising at least one of SEQ ID NOs: 91-94, 100-103, 108-122, 158-192, or a complement thereof, or a variant thereof, or a truncation thereof, or the gRNA may be encoded by or bind and target a polynucleotide sequence comprising at least one of SEQ ID NOs: 76-90, 96-99, 123-157, or a complement thereof, or a variant thereof, or a truncation thereof. A truncation may be 1, 2, 3, 4, 5, 6, 7, 8, or 9 nucleotides shorter than the sequence of the gRNA.

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas9-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas9-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas9-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas9-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

d. Donor Sequence

The CRISPR/Cas9-based gene editing system may include at least one donor sequence. A donor sequence comprises a polynucleotide sequence to be inserted into a genome. A donor sequence may comprise a wild-type sequence of a gene.

The gRNA and donor sequence may be present in a variety of molar ratios. The molar ratio between the gRNA and donor sequence may be 1:1, or 1:15, or from 5:1 to 1:10, or from 1:1 to 1:5. The molar ratio between the gRNA and donor sequence may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the gRNA and donor sequence may be less than 20:1, less than 15:1, less than 10:1, less than 9:1, less than 8:1, less than 7:1, less than 6:1, less than 5:1, less than 4:1, less than 3:1, less than 2:1, or less than 1:1.

e. Repair Pathways

The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### i) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### ii) Non-Homologous End Joining (NHEJ)

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas9-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 3. Reporter Protein

In some embodiments, the DNA targeting compositions or CRISPR/Cas9 systems include at least one reporter protein. A polynucleotide sequence encoding the reporter protein may be operably linked to the polynucleotide sequence encoding the Cas9 protein or Cas9 fusion protein. The reporter protein may include any protein or peptide that is suitably detectable, such as, by fluorescence, chemiluminescence, enzyme activity such as beta galactosidase or alkaline phosphatase, and/or antibody binding detection. The reporter protein may comprise a fluorescent protein. The reporter protein may comprise a protein or peptide detectable with an antibody. For example, the reporter protein may comprise GFP, YFP, RFP, CFP, DsRed, luciferase, and/or Thy1.

### 4. Genetic Constructs

The CRISPR/Cas9-based gene editing system may be encoded by or comprised within one or more genetic constructs. The CRISPR/Cas9-based gene editing system may comprise one or more genetic constructs. The genetic construct, such as a plasmid or expression vector, may comprise a nucleic acid that encodes the CRISPR/Cas9-based gene editing system and/or at least one of the gRNAs. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The vector may also comprise a promoter that is operably linked to the CRISPR/Cas-based gene editing system coding sequence. In some embodiments, the promoter is operably linked to a polynucleotide encoding the Cas9 protein or fusion protein. In some embodiments, the promoter is operably linked to a polynucleotide encoding the at least one gRNA. In some embodiments, the promoter is operably linked to a polynucleotide encoding the Cas9 protein or fusion protein and a polynucleotide encoding the at least gRNA. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas9-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

### 5. Pharmaceutical Compositions

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid: nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

a. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas9 system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-γ producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells.

### 7. Kits

Provided herein is a kit, which may be used to modulate the expression of a gene. The kit comprises genetic constructs or a composition comprising the same, for modulating the expression of a gene, as described above, and instructions for using said composition. In some embodiments, the kit comprises at least one gRNA or a polynucleotide encoding the at least one gRNA. The kit may comprise a Cas9 protein and/or fusion protein, or a polynucleotide encoding the Cas9 protein and/or fusion protein. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof may include a modified AAV vector that includes a gRNA molecule(s) and a Cas9 protein or fusion protein, as described above. The CRISPR/Cas-based gene editing system, as described above, may be included in the kit.

### 8. Methods

a. Methods of Modulating Expression of a Gene

Provided herein are methods of modulating expression of a gene in a cell or subject. The methods may include administering to the cell or the subject a DNA targeting composition as detailed herein, or an isolated polynucleotide sequence as detailed herein, or a vector as detailed herein, or a pharmaceutical composition as detailed herein, or a combination thereof. The expression of the gene may be increased relative to a control. The expression of the gene may be decreased relative to a control. In some embodiments, the gene comprises the dystrophin gene.

b. Methods of Correcting a Mutant Gene

Provided herein are methods of correcting a mutant gene in a cell. The methods may include administering to the cell or the subject a DNA targeting composition as detailed herein, or an isolated polynucleotide sequence as detailed herein, or a vector as detailed herein, or a pharmaceutical composition as detailed herein, or a combination thereof. The methods may further include administering to the cell or subject a donor DNA. In some embodiments, correcting a mutant gene comprises deleting, rearranging, or replacing the mutant gene. In some embodiments, the gene comprises the dystrophin gene.

c. Methods of Treating a Disease

Provided herein are methods of treating a disease in a subject. The methods may include administering to the cell or the subject a DNA targeting composition as detailed herein, or an isolated polynucleotide sequence as detailed herein, or a vector as detailed herein, or a pharmaceutical composition as detailed herein, or a cell as detailed herein, or a combination thereof. The DNA targeting composition, or the isolated polynucleotide sequence, or the vector, or the cell, or the pharmaceutical composition, or a combination thereof, may be administered to skeletal muscle or cardiac muscle of the subject. In some embodiments, the gene comprises the dystrophin gene. In some embodiments, the disease comprises Duchenne muscular dystrophy (DMD) or Becker muscular dystrophy (BMD). In some embodiments, the disease comprises cancer.

### 9. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Cell culture and virus production. HEK293T cells were grown in monolayer on tissue culture plates (Corning) and maintained in DMEM media containing 10% FBS unless otherwise specified. K562 cells were grown in suspension in tissue culture plates (Corning) and maintained in RPMI media containing 10% FBS and 1% penicillin/streptomycin.

Lentivirus was produced in HEK293T cells using Lipofectamine 3000 (Invitrogen, Waltham, MA). HEK293T cells were seeded for transfection and subsequently cultured in OptiMEM with 5% FBS, 1% sodium pyruvate, 1×NEAA, and 1×GlutaMAX. Virus-containing cell culture media was harvested at 24 h and 48 h post-transfection, filtered, and concentrated with LentiX (Takara Bio, San Jose, CA) according to manufacturer protocol. Viral pellets were resuspended in PBS.

All antibiotic selections following lentiviral transduction were started at 48 h post-transduction.

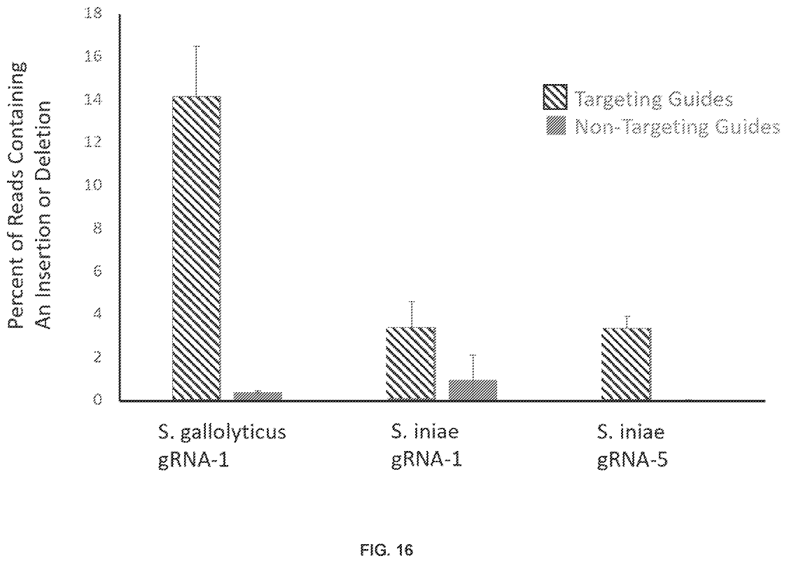

Nuclease assay in mammalian cells. On day 0, HEK293T cells were seeded at a density of 65-105 cells/cm² in a 24-well plate. At 18-30 h after seeding, cells were transfected with SuCas9 nuclease (350 ng) and gRNA (150 ng) plasmids using Lipofectamine 3000 (Invitrogen, Waltham, MA). For results shown in FIG. 16 (Example 10), 250 ng Cas9 nuclease and 250 ng sgRNA plasmid were used. 72 h after transfection, cells were trypsinised and pelleted. Genomic DNA was extracted (Qiagen DNEasy; Qiagen, Hilden, Germany), and regions of interest were PCR amplified from 200 ng gDNA per sample for 25 cycles using KAPA polymerase (Roche, Basel, Switzerland). The PCR product was double size-selected (0.5×, 1×) using Ampure XP beads (Beckman Coulter, Brea, CA). One-fourth of the first PCR product was used in a second PCR of 10 cycles to add sequencing adapters and barcodes. Barcoded samples were pooled and purified again with Ampure XP beads prior to quantification with the Qubit fluorometer assay kit for dsDNA. Pooled libraries were sequenced on an Illumina Miseq (2×150 bp PE; 2×300 bp for results shown in FIG. 16 in Example 10). The resulting sequencing reads were analyzed by determining the length of the region aligning to both sides of the cut site, and any read with greater or less than the expected length of the genomic region was considered to have an insertion or deletion.

In vitro Transcription of sgRNA. For in vitro cleavage reactions, sgRNA were produced by in vitro transcription using the Megashortscript (Thermo Fischer, Waltham, MA) kit according to the manufacturer's instruction for each sgRNA. Template DNA containing the T7 promoter was produced by PCR using primers shows in TABLE 1.

TABLE 1
Primer sequences.

| | |
|---|---|
| spCas9_NT_ | aagcTAATACGACTCACTATAGGGTGTCGTGATGCG |
| guide_T7_DNA | TAGACGGGTTTAAGAGCTATGCTGGAAACAGCATAG |
| | CAAGTTTAAATAAGGCTAGTCCGTTATCAACTTGAA |
| | AAAGTGGCACCGAGTCGGTGCTTTTTTT |
| | (SEQ ID NO: 72) |
| spCas9_Trac_ | aagcTAATACGACTCACTATAGGCTTCAAGAGCAAC |
| guide_T7_DNA | AGTGCTGGTTTAAGAGCTATGCTGGAAACAGCATAG |
| | CAAGTTTAAATAAGGCTAGTCCGTTATCAACTTGAA |
| | AAAGTGGCACCGAGTCGGTGCTTTTTTT |
| | (SEQ ID NO: 73) |
| suCas9_NT_ | aagcTAATACGACTCACTATAGGGTGTCGTGATGCG |
| guide_T7_DNA | TAGACGGGTTTTTGTACTCTCAAGATTTCGAAAAAT |
| | CTTGCTGAGCCTACAAAGATAAGGCTTCATGCCGAA |
| | TTCAAGCACCCCATCATTGATGGGGTGCTTTTCGTA |
| | TT |
| | (SEQ ID NO: 74) |
| suCas9_TRAC_ | aagcTAATACGACTCACTATAGGTGTTTGAGAATCA |
| guide_T7_DNA | AAATCGGGTTTTTGTACTCTCAAGATTTCGAAAAAT |
| | CTTGCTGAGCCTACAAAGATAAGGCTTCATGCCGAA |
| | TTCAAGCACCCCATCATTGATGGGGTGCTTTTCGTA |
| | TT |
| | (SEQ ID NO: 75) |

### Example 2

### S. uberis and S. Pyogenes Cas9 Protein Purification

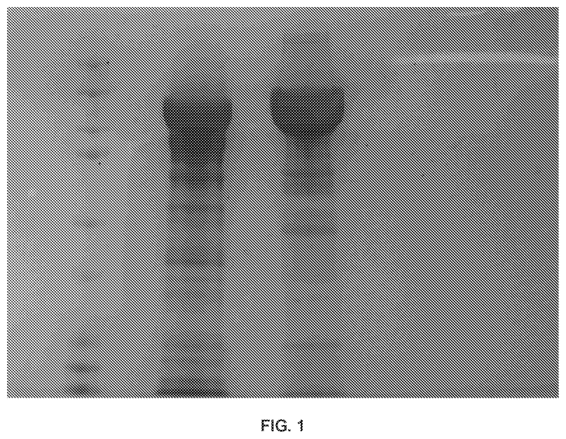

BL21 E. coli cells (Millipore EMD; MilliporeSigma, Burlington, MA) were transformed with Cas9 expression plasmid and plated on plates containing appropriate antibiotics. Liquid cultures were then inoculated and allowed to grow at 37° C. until the OD600 was 0.6 to 0.8, and then induced with 0.5 mM IPTG and grown overnight at 18° C. Cultures were then pelleted by centrifugation (10 min at 4000×g). Cells were resuspended in lysis buffer, lysed by sonication, and spun at 24000×g to remove cell debris. The lysate was then flowed over a 2 mL bed volume of Ni-NTA agarose (Qiagen, Hilden, Germany), washed twice with wash buffer, and then once with wash buffer without triton. Protein was eluted by the addition of 5 mL elution buffer. Eluted protein was dialyzed into exchange buffer, and concentration was determined by A280. The buffers used for protein purification included the following: Lysis Buffer (20 mM Tris-HCl PH 8.0, 500 mM NaCl, 20 mM imidazole, 5% glycerol, 1 mg/mL lysozyme, 1 tablet Complete protease inhibitor, EDTA-free); Wash Buffer (20 mM Tris-HCl PH 8.0, 500 mM NaCl, 30 mM imidazole, 0.5% triton x-100); Elution Buffer (20 mM Tris-HCl PH 8.0, 500 mM NaCl, 250 mM imidazole); and Exchange Buffer (20 mM Tris-HCl PH 7.5, 250 mM NaCl). An SDS-PAGE gel of purified SuCas9 and SpCas9 is shown in FIG. 1. SuCas9 is 138 kDa, and SpCas9 is 160 kDa.

### Example 3

### In Vivo PAM Determination Assay for SuCas9

PAM library construction. A plasmid library containing a region of 7 randomized bases was generated as previously described (Maxwell et al., Methods 2018, 143, 48-57, incorporated herein by reference). Briefly, the NEBuilder HiFi DNA Assembly Master Mix (NEB, Ipswich, MA) was used according to the manufacturer's instructions to assemble a PCR-amplified gBlock (IDT, Coralville, IA) containing the randomized bases and a PCR-amplified backbone containing a ColA replication of origin and kanamycin resistance gene. The assembled plasmids were purified and concentrated using the Monarch PCR & DNA Cleanup Kit (NEB, Ipswich, MA) and transformed into NEB 10-beta Electrocompetent E. coli (NEB, Ipswich, MA). Following recovery, a portion of the culture was serially diluted and plated on LB agar plates supplemented with 50 μg/mL kanamycin to calculate transformation efficiency. The remaining cells were back-diluted in LB with 50 μg/mL kanamycin, grown overnight, and used for glycerol stocks and plasmid Midiprep (Qiagen, Hilden, Germany). The result was the 7-mer random base PAM library.

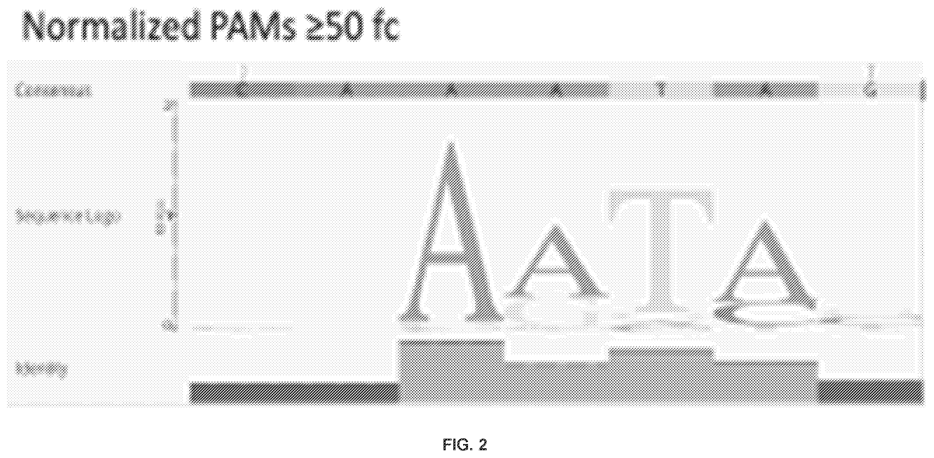

Transformation-based PAM library assay. To verify the predicted PAM and identify possible flexibility in the PAM sequence for SuCas9, nuclease activity for SuCas9 was assessed in E. coli with the 7-mer random base PAM library downstream of the protospacer sequence. Electrocompetent E. coli BL21 (DE3) cells (Sigma-Aldrich, St. Louis, MO) were transformed with 50 ng each of the S. uberis Cas9/sgRNA expression plasmid (pACYCduet_uberis_t7_pam) and the 7N plasmid library (pMAC223_L). Following a 1-hour recovery at 37° C. and 250 RPM, cells were plated at low density on LB agar plates supplemented with 50 μg/mL kanamycin, 34 μg/mL chloramphenicol, and 0.1 mM IPTG and incubated overnight at 37° C. Approximately 60,000-80,000 surviving colonies were scraped from the plates, and plasmid DNA was isolated by Midiprep (Qiagen, Hilden, Germany). As shown in FIG. 2, the consensus PAM was determined to be NNAATA, with possible flexibility at positions 4 and 6 (G and C, respectively).

### Example 4

### Protospacer Length Optimization for SuCas9

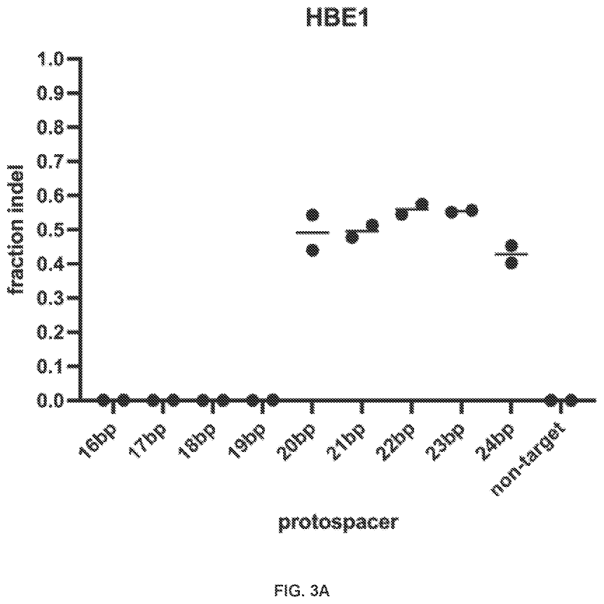

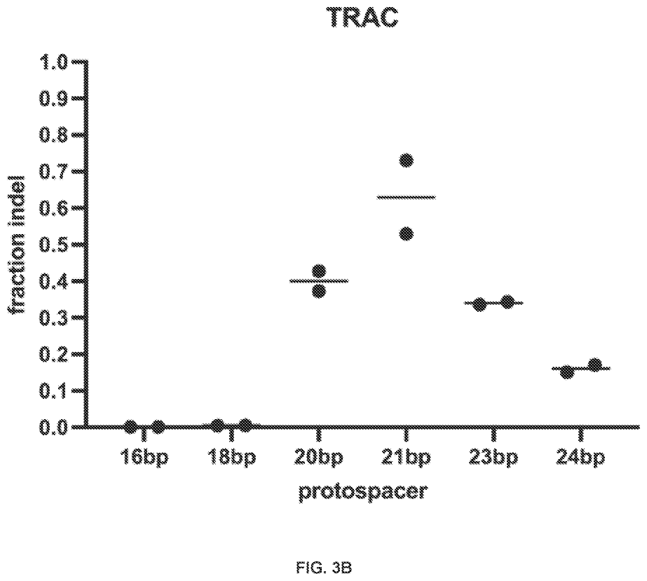

To determine the optimal protospacer length for SuCas9 nuclease, indel frequency was assessed for varying gRNA protospacer lengths for two gene targets, HBE1 and TRAC, in mammalian cells. Results are shown in FIG. 3A and FIG. 3B. The sgRNA protospacer sequences are shown in TABLE 2.

TABLE 2
sgRNA protospacer sequences.

| | |
|---|---|
| Length | Protospacer Sequence |

TRAC protospacer sequences:

| | |
|---|---|
| 24 bp | GCATTTGTTTGAGAATCAAAATCGG (SEQ ID NO: 76) |
| | GCATTTGTTTGAGAATCAAAATCGG (SEQ ID NO: 108) |
| 23 bp | GATTTGTTTGAGAATCAAAATCGG (SEQ ID NO: 77) |
| | GATTTGTTTGAGAATCAAAATCGG (SEQ ID NO: 109) |
| 21 bp | GTTGTTTGAGAATCAAAATCGG (SEQ ID NO: 78) |
| | GTTGTTTGAGAATCAAAATCGG (SEQ ID NO: 110) |
| 20 bp | GTGTTTGAGAATCAAAATCGG (SEQ ID NO: 79) |
| | GTGTTTGAGAATCAAAATCGG (SEQ ID NO: 111) |
| 18 bp | GTTTGAGAATCAAAATCGG (SEQ ID NO: 80) |
| | GTTTGAGAATCAAAATCGG (SEQ ID NO: 112) |
| 16 bp | GTGAGAATCAAAATCGG (SEQ ID NO: 81) |
| | GTGAGAATCAAAATCGG (SEQ ID NO: 113) |

HBE protospacer sequences:

| | |
|---|---|
| 24 bp | GTTCTCAATGCATGGGAATGAAGGG (SEQ ID NO: 82) |
| | GTTCTCAATGCATGGGAATGAAGGG (SEQ ID NO: 114) |
| 23 bp | GTCTCAATGCATGGGAATGAAGGG (SEQ ID NO: 83) |
| | GTCTCAATGCATGGGAATGAAGGG (SEQ ID NO: 115) |
| 22 bp | GCTCAATGCATGGGAATGAAGGG (SEQ ID NO: 84) |
| | GCTCAATGCATGGGAATGAAGGG (SEQ ID NO: 116) |
| 21 bp | GTCAATGCATGGGAATGAAGGG (SEQ ID NO: 85) |
| | GTCAATGCATGGGAATGAAGGG (SEQ ID NO: 117) |
| 20 bp | GCAATGCATGGGAATGAAGGG (SEQ ID NO: 86) |
| | GCAATGCATGGGAATGAAGGG (SEQ ID NO: 118) |
| 19 bp | GAATGCATGGGAATGAAGGG (SEQ ID NO: 87) |
| | GAATGCATGGGAATGAAGGG (SEQ ID NO: 119) |
| 18 bp | GATGCATGGGAATGAAGGG (SEQ ID NO: 88) |
| | GATGCATGGGAATGAAGGG (SEQ ID NO: 120) |
| 17 bp | GTGCATGGGAATGAAGGG (SEQ ID NO: 89) |
| | GTGCATGGGAATGAAGGG (SEQ ID NO: 121) |
| 16 bp | GGCATGGGAATGAAGGG (SEQ ID NO: 90) |
| | GGCATGGGAATGAAGGG (SEQ ID NO: 122) |

### Example 5

### In Vitro Cleavage Reaction

Purified SuCas9 or SpCas9 protein was complexed with the in vitro transcribed sgRNA that either targeted or did not target the DNA amplicon. The sgRNA sequences 6, 7, 8, and 9 are in the gel left to right and shown in TABLE 3. Successful SuCas9 cutting was expected to generate fragments of approximately 100 bp and 300 bp, while successful SpCas9 cutting was expected to generate fragments of approximately 200 bp and 190 bp.

TABLE 3
sgRNA sequences for in vivo cleavage reaction.

| | |
|---|---|
| 6. Uberis_TRAC1_ | UGUUUGAGAAUCAAAAUCGGGUUUUUGUACUC |
| sgRNA_RNA | UCAAGAUUUCGAAAAAUCUUGCUGAGCCUACA |
| | AAGAUAAGGCUUCAUGCCGAAUUCAAGCACCC |
| | CAUCAUUGAUGGGGUGCUUUUCGUAUU |
| | (SEQ ID NO: 91) |
| 7. Uberis_NT_ | GUGUCGUGAUGCGUAGACGGGUUUUUGUACUC |
| sgRNA_RNA | UCAAGAUUUCGAAAAAUCUUGCUGAGCCUACA |
| | AAGAUAAGGCUUCAUGCCGAAUUCAAGCACCC |
| | CAUCAUUGAUGGGGUGCUUUUCGUAUU |
| | (SEQ ID NO: 92) |
| 8. Pyogenes_ | CUUCAAGAGCAACAGUGCUGGUUUAAGAGCUA |
| TRAC1_sgRNA_RNA | UGCUGGAAACAGCAUAGCAAGUUUAAAUAAGG |
| | CUAGUCCGUUAUCAACUUGAAAAAGUGGCACC |
| | GAGUCGGUGCUUUUUUU |
| | (SEQ ID NO: 93) |
| 9. Pyogenes_NT_ | GUGUCGUGAUGCGUAGACGGGUUUAAGAGCUA |
| sgRNA_RNA | UGCUGGAAACAGCAUAGCAAGUUUAAAUAAGG |
| | CUAGUCCGUUAUCAACUUGAAAAAGUGGCACC |
| | GAGUCGGUGCUUUUUUU |
| | (SEQ ID NO: 94) |

In a 20 μL reaction, 100 nM Cas9 protein and 100 nM sgRNA were first pre-complexed for 10 minutes in exchange buffer supplemented with 10 μM MgCl₂, and the target DNA amplicon

(GTCTCGTGGGCTCGGAGATGTGTATAAGAGACAGCCTGACCCTGCCGTG
TACCAGCTGAGAGACTCTAAATCCAGTGACAAGTCTGTCTGCCTATTCAC
CGATTTTGATTCTCAAACAAATGTGTCACAAAGTAAGGATTCTGATGTGT
ATATCACAGACAAAACTGTGCTAGACATGAGGTCTATGGACTTCAAGAGC
AACAGTGCTGTGGCCTGGAGCAACAAATCTGACTTTGCATGTGCAAACGC
CTTCAACAACAGCATTATTCCAGAAGACACCTTCTTCCCCAGCCCAGGTA
AGGGCAGCTTTGGTGCCTTCGCAGGCTGTTTCCTTGCTTCAGGAATGGCC
AGGTTCTGCCCAGAGCCTGTCTCTTATACACATCTGACGCTGCCGACGA,
SEQ ID NO: 95)

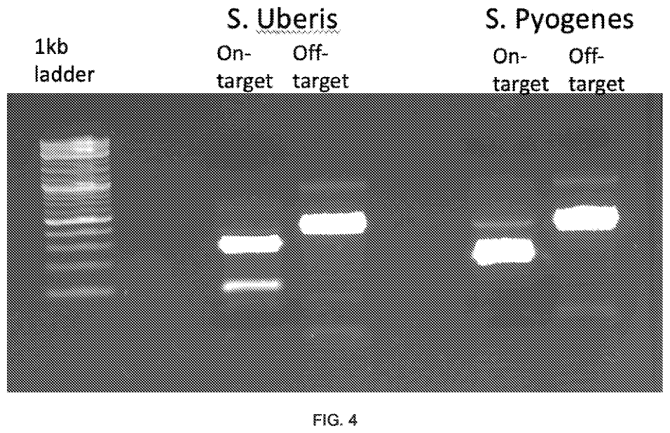

was then added to a final concentration of 6 μM. Reactions with sgRNA that did not target the DNA amplicon were used as a control. The target amplicon was generated by PCR from the TRAC gene in 293T cells. Reactions were incubated at 37° C. for two hours and then terminated by the addition of proteinase K (500 μg/mL final concentration) followed by a 10-minute incubation at 37° C. Reactions were then analyzed on a 1% agarose gel (FIG. 4). The results showed that SuCas9 ribonucleoprotein complexes were generated and that SuCas9 has activity similar to SpCas9.

### Example 6

### S. uberis dCas9-KRAB Repression Assays in Mammalian Cells

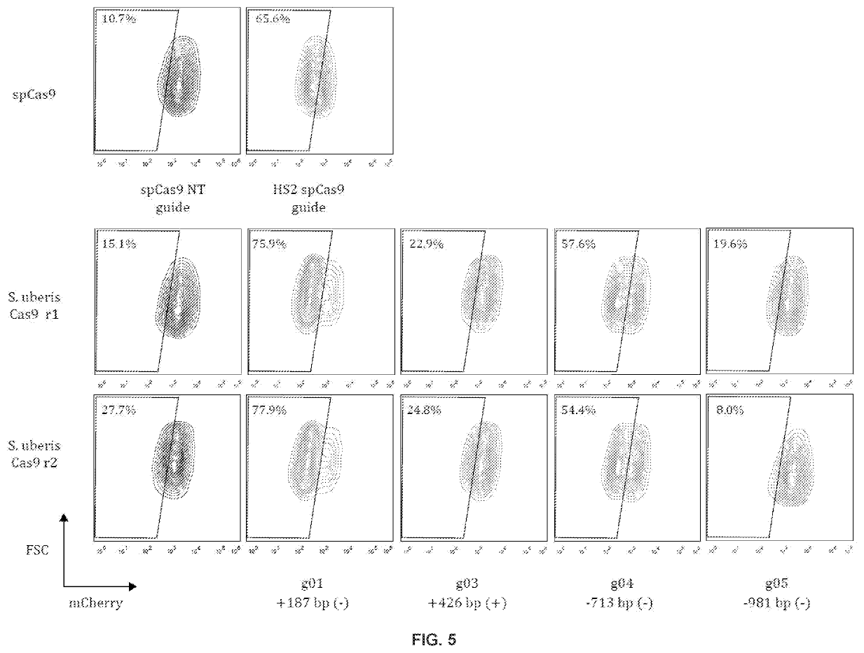

The K562 HBE-mCherry reporter cell line (generated by Klann et al., Nature Biotechnology 2017, 35, 561-568, incorporated herein by reference) contained mCherry fluorescent protein sequence inserted at the 3′ end of the HBE gene. The K562 HBE-mCherry reporter cell line was used to test gene repression activity of Su-dCas9-KRAB with gRNAs targeting the HBE promoter (TABLE 4). K562 HBE-mCherry cells were transduced with S. uberis dCas9-KRAB or S. pyogenes dCas9-KRAB lentivirus (in a cassette containing a blasticidin resistance gene) and selected with 5 μg/mL blasticidin for 5 days to create a stable cell line. The stable dCas9-KRAB line was further transduced with individual gRNA lentivirus (single gRNAs in a cassette containing a puromycin resistance gene), and selected with puromycin for 72 h. Then 9 or 10 days post-transduction, cells were harvested and analyzed for mCherry expression on a flow cytometer (Sony SH800). Results are shown in FIG. 5, showing that S. uberis dCas9-KRAB mediated repression of the fluorescent HBE reporter.

TABLE 4
Guide RNA (gRNA) sequences for HBE-mCherry assay with S. uberis Cas9.

| Name | DNA | RNA |
|---|---|---|
| g01 | CAATGCATGGGAATGAAGGGGTTTTTG | CAAUGCAUGGGAAUGAAGGGGUUUUUGU |
| | TACTCTCAAGATTTCGAAAAATCTTGC | ACUCUCAAGAUUUCGAAAAAUCUUGCUG |
| | TGAGCCTACAAAGATAAGGCTTCATGC | AGCCUACAAAGAUAAGGCUUCAUGCCGA |
| | CGAATTCAAGCACCCCATCATTGATGG | AUUCAAGCACCCCAUCAUUGAUGGGGUG |
| | GGTGCTTTTCGTATT (SEQ ID NO: 96) | CUUUUCGUAUU (SEQ ID NO: 100) |
| g03 | GCTTGAGGTTGTCCATGTTTGTTTTTG | GCUUGAGGUUGUCCAUGUUUGUUUUUGU |
| | TACTCTCAAGATTTCGAAAAATCTTGC | ACUCUCAAGAUUUCGAAAAAUCUUGCUG |
| | TGAGCCTACAAAGATAAGGCTTCATGC | AGCCUACAAAGAUAAGGCUUCAUGCCGA |
| | CGAATTCAAGCACCCCATCATTGATGG | AUUCAAGCACCCCAUCAUUGAUGGGGUG |
| | GGTGCTTTTCGTATT (SEQ ID NO: 97) | CUUUUCGUAUU (SEQ ID NO: 101) |
| g04 | AAGCAAGAAGAGAGCCCCAGGTTTTTG | AAGCAAGAAGAGAGCCCCAGGUUUUUGU |
| | TACTCTCAAGATTTCGAAAAATCTTGC | ACUCUCAAGAUUUCGAAAAAUCUUGCUG |
| | TGAGCCTACAAAGATAAGGCTTCATGC | AGCCUACAAAGAUAAGGCUUCAUGCCGA |
| | CGAATTCAAGCACCCCATCATTGATGG | AUUCAAGCACCCCAUCAUUGAUGGGGUG |
| | GGTGCTTTTCGTATT (SEQ ID NO: 98) | CUUUUCGUAUU (SEQ ID NO: 102) |
| g05 | TTCAGGCACATGGATCGAATGTTTTTG | UUCAGGCACAUGGAUCGAAUGUUUUUGU |
| | TACTCTCAAGATTTCGAAAAATCTTGC | ACUCUCAAGAUUUCGAAAAAUCUUGCUG |
| | TGAGCCTACAAAGATAAGGCTTCATGC | AGCCUACAAAGAUAAGGCUUCAUGCCGA |
| | CGAATTCAAGCACCCCATCATTGATGG | AUUCAAGCACCCCAUCAUUGAUGGGGUG |
| | GGTGCTTTTCGTATT (SEQ ID NO: 99) | CUUUUCGUAUU (SEQ ID NO: 103) |

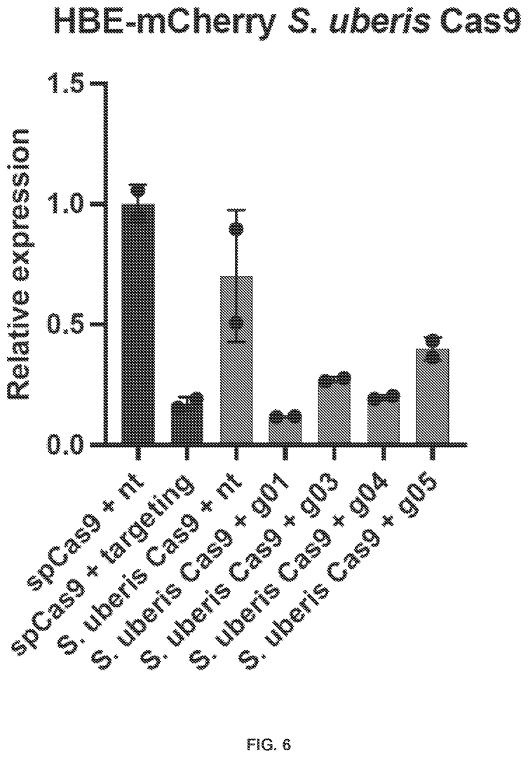

To verify repression of HBE-mCherry at the transcript level with the novel DNA targeting system, RNA was extracted from remaining cells (Norgen Total RNA Plus or Qiagen RNEasy Plus; Qiagen, Hilden, Germany). HBE gene expression was analyzed with qPCR using primers targeting HBE as listed in TABLE 5 (PerfeCTa SYBR Green Fastmix, Quantabio, Beverly, MA). Results are shown in FIG. 6, showing that S. uberis dCas9-KRAB mediated repression of HBE mRNA expression.

TABLE 5
Sequencing primer sequences.

| | |
|---|---|
| HBE forward | TCGTCGGCAGCGTCAGATGTGTATAAGAGACAGGAGT |
| | CTATGAAATGACACCATATC |
| | (SEQ ID NO: 104) |
| HBE reverse | GTCTCGTGGGCTCGGAGATGTGTATAAGAGACAGTCA |
| | CTAGCCTGTGGAG |
| | (SEQ ID NO: 105) |
| TRAC forward | TCGTCGGCAGCGTCAGATGTGTATAAGAGACAGCCTG |
| | ACCCTGCCGTGTACCA |
| | (SEQ ID NO: 106) |
| TRAC reverse | GTCTCGTGGGCTCGGAGATGTGTATAAGAGACAGGCT |
| | CTGGGCAGAACCTGGCC |
| | (SEQ ID NO: 107) |

### Example 7

### Gene Activation with S. uberis dCas9-p300

A fusion protein of S. uberis dCas9-p300 was tested for gene activation in HEK293T cells. S. uberis dCas9-p300 (SU) or S. pyogenes dCas9-p300 (SP, as a positive control) were studied with appropriate gRNAs targeting the promoter of HBG1 or IL1RN. HEK293T cells were plated at circa 105,000 cells/cm² (200,000 cells/well in a 24 well plate) 1 day prior to transfection. Cells were transfected with plasmids encoding dCas9-p300 (350 ng/well) and gRNA (150 ng/well) with Lipofectamine 3000 (Invitrogen) following manufacturer recommendations. Cells were harvested at 72 hours after transfection (Norgen Total RNA Plus). Relative mRNA expression was quantified with RT-qPCR (Quantabio PerfeCTa SYBR Green Fastmix).

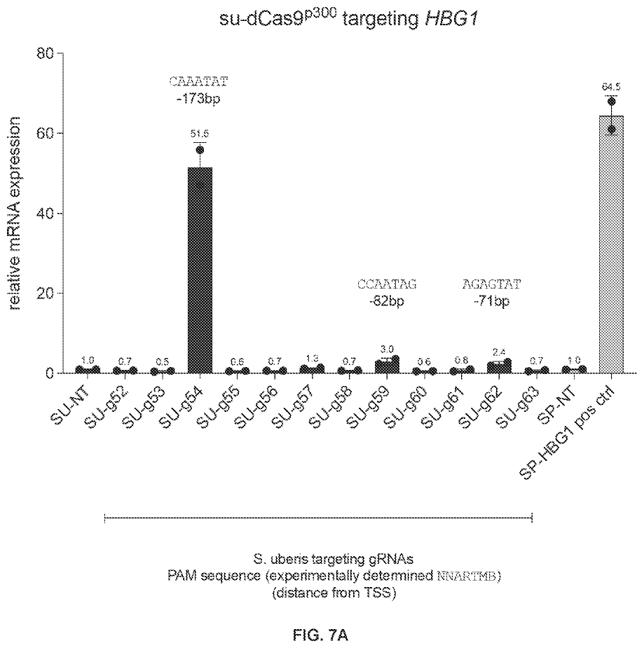

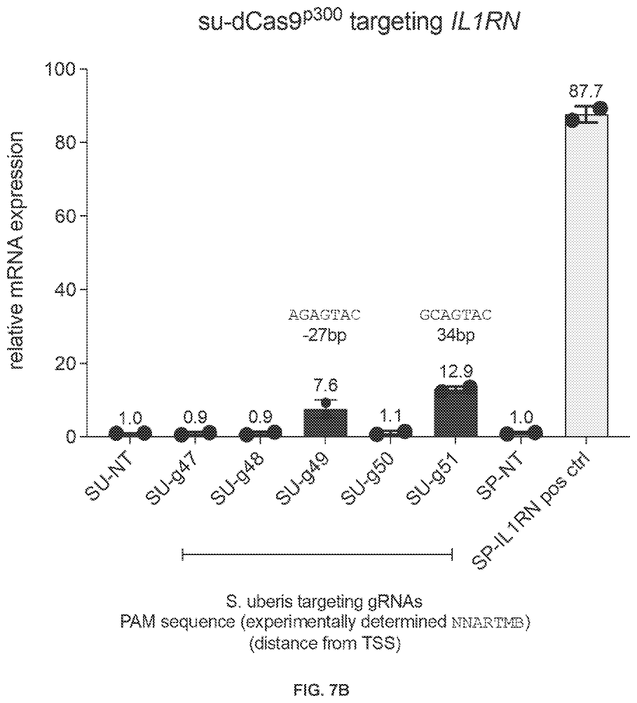

The results showed that S. uberis dCas9-p300 fusion activated target genes HBG1 (FIG. 7A) and IL1RN (FIG. 7B) in HEK293T cells. PAM sequences and distances from TSS are indicated above select gRNAs. Negative numbers indicated gRNA position upstream of TSS on transcribed strand.

### Example 8

### PAM Sequence Determination

The PAM sequence for each new Cas9 protein was determined. Individual 12 μL TXTL reactions were assembled consisting of 9.375 μL myTXTL Linear DNA Master Mix (Daicel Arbor Biosciences), 0.5 mM IPTG, 0.2 nM pTXTL-P70a-T7rnap (Daicel Arbor Biosciences), 2 nM Cas9 linear DNA containing a T7 promoter and 2 nM linear sgRNA expression gBlock, and 0.5 nM 7N plasmid library. TXTL reactions were incubated at 29° C. for 16 hours. The DNA was purified from the TXTL reactions with the Monarch PCR & DNA Cleanup Kit (NEB). DNA libraries were then amplified by PCR and subjected to Illumina sequencing to determine counts of each sequence.

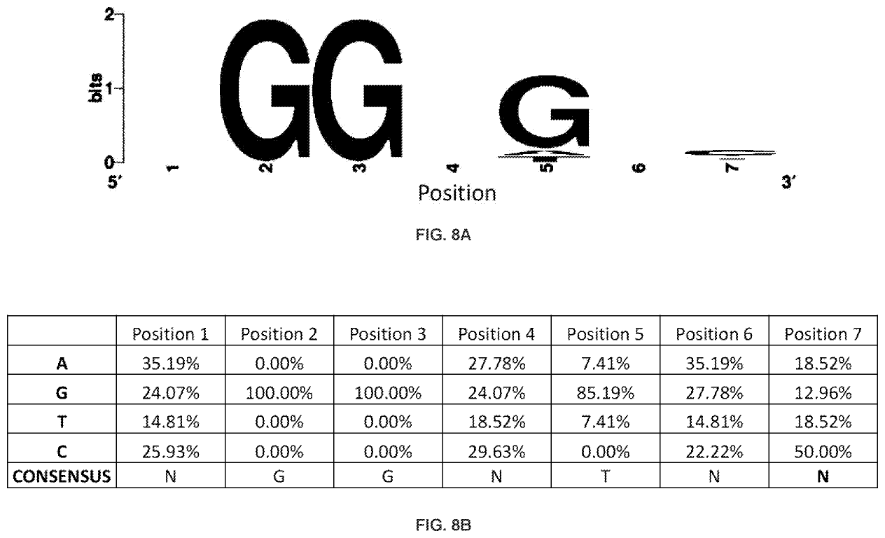

Results of the empirical PAM determination for S. dysgalactiae Cas9 are shown in FIGS. 8A-8B. FIG. 8A is the sequence logo produced for all sequences depleted a minimum of 10-fold in the empirical PAM determination assay. FIG. 8B is a table showing the percent of depleted sequences containing each nucleotide at each position. Positions 1-7 are the nucleotides directly following the protospacer in the target genome. The allowed PAM sequence for S. dysgalactiae Cas9 was found to be NNGGNTN for S. dysgalactiae Cas9, with a slight preference for C in the final position.

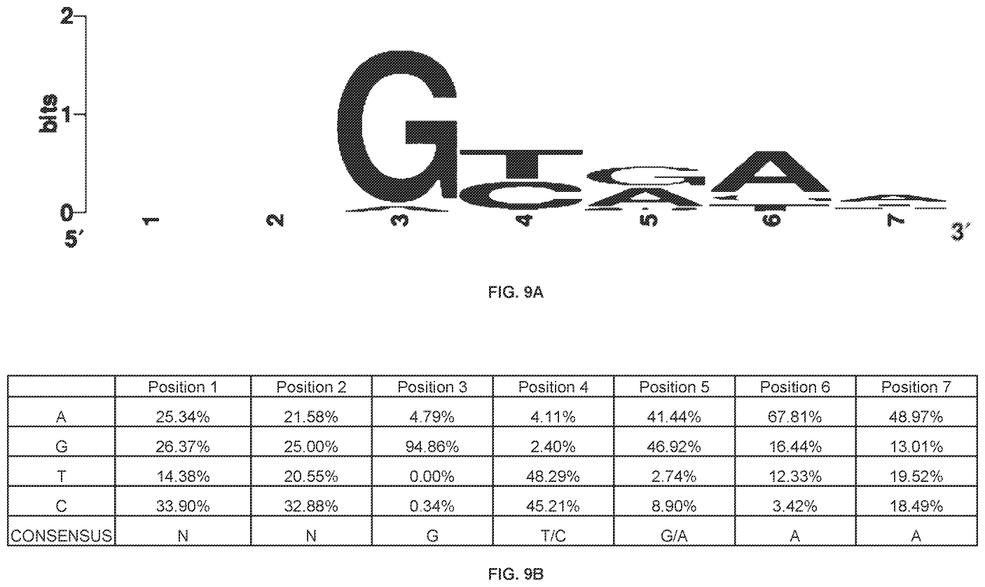

Results of empirical PAM determination for S. gallolyticus Cas9 are shown in FIGS. 9A-9B. FIG. 9A is the sequence logo produced for all sequences depleted a minimum of 10-fold in the empirical PAM determination assay. FIG. 9B is a table showing the percent of depleted sequences containing each nucleotide at each position. Positions 1-7 are the nucleotides directly following the protospacer in the target genome. The allowed PAM sequence for S. gallolyticus Cas9 was found to be NNG(T/C)(G/A)AN, with a slight preference for A in the final position.

Results of empirical PAM determination for S. iniae Cas9 are shown in FIGS. 10A-10B. FIG. 10A is the sequence logo produced for all sequences depleted a minimum of 10-fold in the empirical PAM determination assay. FIG. 10B is a table showing the percent of depleted sequences containing each nucleotide at each position. Positions 1-7 are the nucleotides directly following the protospacer in the target genome. The allowed PAM sequence for S. iniae Cas9 was found to be NNGGNNN.

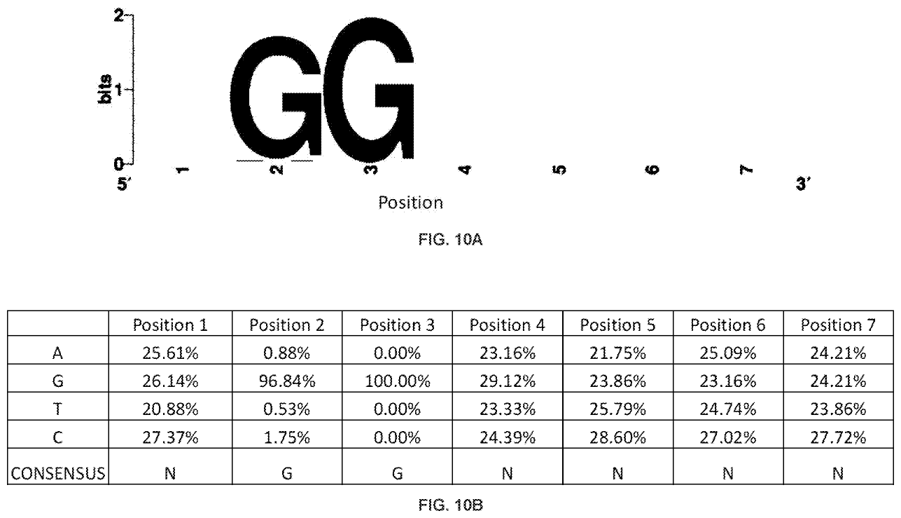

Results of empirical PAM determination for S. lutetiensis Cas9 are shown in FIG. 11A-11B. FIG. 11A is the sequence logo produced for all sequences depleted a minimum of 10-fold in the empirical PAM determination assay. FIG. 11B is a table showing the percent of depleted sequences containing each nucleotide at each position. Positions 1-7 are the nucleotides directly following the protospacer in the target genome. The allowed PAM sequence for S. lutetiensis Cas9 was found to be NNAAAAN with a slight preference for A at the final position.

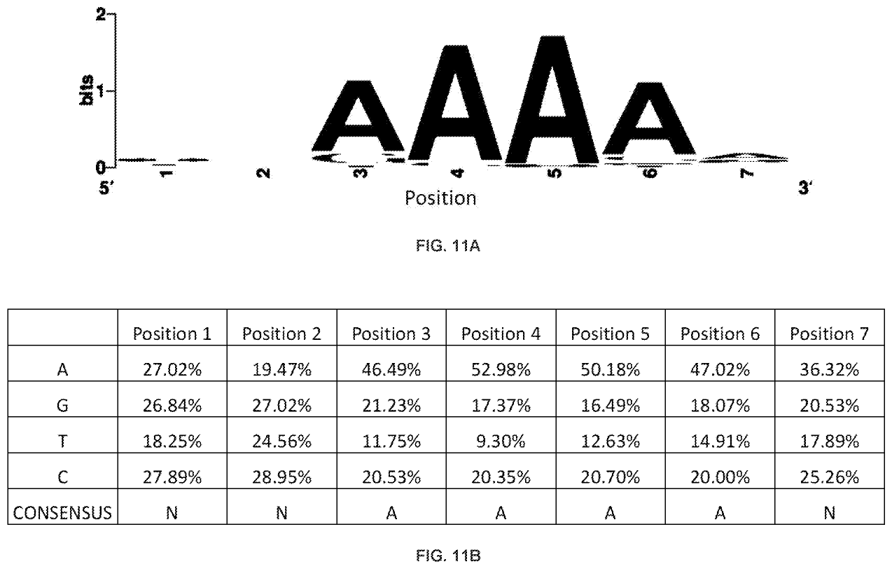

Results of empirical PAM determination for S. parasanguinis Cas9 are shown in FIG. 12A-12B. FIG. 12A is the sequence logo produced for all sequences depleted a minimum of 10-fold in the empirical PAM determination assay. FIG. 12B is a table showing the percent of depleted sequences containing each nucleotide at each position. Positions 1-7 are the nucleotides directly following the protospacer in the target genome. The allowed PAM sequence for S. parasanguinis Cas9 was found to be NNAA(A/G)GN with a slight preference for G, C, or T at the final position.

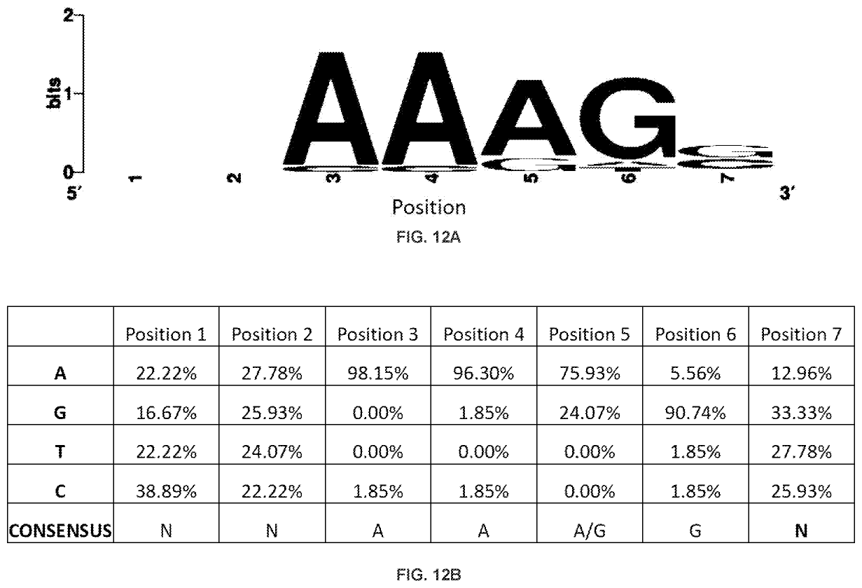

Results of empirical PAM determination for S. uberis Cas9 are shown in FIG. 13A-13B. FIG. 13A is the sequence logo produced for all sequences depleted a minimum of 10-fold in the empirical PAM determination assay. FIG. 13B is a table showing the percent of depleted sequences containing each nucleotide at each position. Positions 1-7 are the nucleotides directly following the protospacer in the target genome. The allowed PAM sequence for S. uberis Cas9 was found to be NNA(A/G)TAN with a slight preference for G, C, or T at the final position.

### Example 9

### Gene Repression with Various dCas9-KRAB Fusion Proteins

dCas9-KRAB fusion proteins were generated with dCas9 proteins from various species, and the fusion proteins were tested for gene expression repression. The K562 HBE-mCherry reporter cell line (generated by Klann et al., Nature Biotechnology 2017, 35, 561-568, incorporated herein by reference) contained mCherry fluorescent protein sequence inserted at the 3′ end of the HBE gene. The K562 HBE-mCherry reporter cell line was used to test gene repression activity of dCas9-KRAB with gRNAs targeting the HBE promoter for various different dCas9 proteins. K562 HBE-mCherry cells were transduced with dCas9-KRAB lentivirus (in a cassette containing a GFP gene), and the resulting dCas9-KRAB line was further transduced with pooled sgRNA lentivirus. Alternatively, the K562 HBE-mCherry cells were lentivirally transduced with the dCas9-KRAB in a cassette containing a blasticidin resistance gene, cells were selected with blasticidin for 5 days to create a stable line, Cas9-containing cells were lentivirally transduced with single gRNAs in a cassette containing a puromycin resistance gene, and the cells were cultured for 10 days with puromycin selection on days 3-6. There were 2 to 5 gRNAs targeting the HBE TSS per PAM. Cells were harvested and assayed for mCherry repression by flow cytometry. 10 days post-transduction, GFP positive transduced cells were harvested and analyzed for mCherry expression on a flow cytometer (Sony SH800). The gRNA sequences used are shown in TABLE 6.

TABLE 6
Guide RNA (gRNA) sequences targeting HBE for HBE-mCherry assay with
dCas9 from various species.

| Species of dCas9 | protospacer targeting HBE-DNA | protospacer targeting HBE-RNA |
|---|---|---|
| Streptococcus agalactiae | GACTCCTCGTTGTTTACCCC | GACUCCUCGUUGUUUACCCC |
| | (SEQ ID NO: 123) | (SEQ ID NO: 158) |
| Streptococcus agalactiae | ATTACCCTAGCAAGTTGATT | AUUACCCUAGCAAGUUGAUU |
| | (SEQ ID NO: 124) | (SEQ ID NO: 159) |
| Streptococcus agalactiae | TATTACCCTAGCAAGTTGAT | UAUUACCCUAGCAAGUUGAU |
| | (SEQ ID NO: 125) | (SEQ ID NO: 160) |
| Streptococcus agalactiae | CTTCGGCAGTAAAGAATAAA | CUUCGGCAGUAAAGAAUAAA |
| | (SEQ ID NO: 126) | (SEQ ID NO: 161) |
| Streptococcus agalactiae | GCTAGTGATTGCAGCTGTGT | GCUAGUGAUUGCAGCUGUGU |
| | (SEQ ID NO: 127) | (SEQ ID NO: 162) |
| Streptococcus gallolyticus | ACAGGGGGCCAGAACTTCGG | ACAGGGGGCCAGAACUUCGG |
| | (SEQ ID NO: 128) | (SEQ ID NO: 163) |
| Streptococcus gallolyticus | CAAAAAATCTCTGGGTCCAG | CAAAAAAUCUCUGGGUCCAG |
| | (SEQ ID NO: 129) | (SEQ ID NO: 164) |
| Streptococcus gallolyticus | GACGGCAGCCTTCTCCTCAG | GACGGCAGCCUUCUCCUCAG |
| | (SEQ ID NO: 130) | (SEQ ID NO: 165) |
| Streptococcus gallolyticus | TAGCTCTCTTAAGGAGTGCA | UAGCUCUCUUAAGGAGUGCA |
| | (SEQ ID NO: 131) | (SEQ ID NO: 166) |
| Streptococcus gallolyticus | CATGGATCGAATTGAATACA | CAUGGAUCGAAUUGAAUACA |
| | (SEQ ID NO: 132) | (SEQ ID NO: 167) |
| Streptococcu iniae | GACTCCTCGTTGTTTACCCC | GACUCCUCGUUGUUUACCCC |
| | (SEQ ID NO: 133) | (SEQ ID NO: 168) |
| Streptococcu iniae | ATTACCCTAGCAAGTTGATT | AUUACCCUAGCAAGUUGAUU |
| | (SEQ ID NO: 134) | (SEQ ID NO: 169) |
| Streptococcu iniae | TATTACCCTAGCAAGTTGAT | UAUUACCCUAGCAAGUUGAU |
| | (SEQ ID NO: 135) | (SEQ ID NO: 170) |
| Streptococcu iniae | CTTCGGCAGTAAAGAATAAA | CUUCGGCAGUAAAGAAUAAA |
| | (SEQ ID NO: 136) | (SEQ ID NO: 171) |
| Streptococcu iniae | GCTAGTGATTGCAGCTGTGT | GCUAGUGAUUGCAGCUGUGU |
| | (SEQ ID NO: 137) | (SEQ ID NO: 172) |
| Streptococcus lutetiensis | TGCAGATAGATGAGGAGCCA | UGCAGAUAGAUGAGGAGCCA |
| | (SEQ ID NO: 138) | (SEQ ID NO: 173) |
| Streptococcus lutetiensis | GCAGATAGATGAGGAGCCAA | GCAGAUAGAUGAGGAGCCAA |
| | (SEQ ID NO: 139) | (SEQ ID NO: 174) |
| Streptococcus lutetiensis | CGACAGGTTTCCAAAGCTGT | CGACAGGUUUCCAAAGCUGU |
| | (SEQ ID NO: 140) | (SEQ ID NO: 175) |
| Streptococcus lutetiensis | TCAGATACAAAATTAGAGAT | UCAGAUACAAAAUUAGAGAU |
| | (SEQ ID NO: 141) | (SEQ ID NO: 176) |
| Streptococcus lutetiensis | CAGATACAAAATTAGAGATG | CAGAUACAAAAUUAGAGAUG |
| | (SEQ ID NO: 142) | (SEQ ID NO: 177) |
| Streptococcus mutans | GACTCCTCGTTGTTTACCCC | GACUCCUCGUUGUUUACCCC |
| | (SEQ ID NO: 143) | (SEQ ID NO: 178) |
| Streptococcus mutans | ATTACCCTAGCAAGTTGATT | AUUACCCUAGCAAGUUGAUU |
| | (SEQ ID NO: 144) | (SEQ ID NO: 179) |
| Streptococcus mutans | TATTACCCTAGCAAGTTGAT | UAUUACCCUAGCAAGUUGAU |
| | (SEQ ID NO: 145) | (SEQ ID NO: 180) |
| Streptococcus mutans | CTTCGGCAGTAAAGAATAAA | CUUCGGCAGUAAAGAAUAAA |
| | (SEQ ID NO: 146) | (SEQ ID NO: 181) |
| Streptococcus mutans | GCTAGTGATTGCAGCTGTGT | GCUAGUGAUUGCAGCUGUGU |
| | (SEQ ID NO: 147) | (SEQ ID NO: 182) |
| Streptococcus parauberis | GACTCCTCGTTGTTTACCCC | GACUCCUCGUUGUUUACCCC |
| | (SEQ ID NO: 148) | (SEQ ID NO: 183) |
| Streptococcus parauberis | ATTACCCTAGCAAGTTGATT | AUUACCCUAGCAAGUUGAUU |
| | (SEQ ID NO: 149) | (SEQ ID NO: 184) |
| Streptococcus parauberis | TATTACCCTAGCAAGTTGAT | UAUUACCCUAGCAAGUUGAU |
| | (SEQ ID NO: 150) | (SEQ ID NO: 185) |
| Streptococcus parauberis | CTTCGGCAGTAAAGAATAAA | CUUCGGCAGUAAAGAAUAAA |
| | (SEQ ID NO: 151) | (SEQ ID NO: 186) |
| Streptococcus parauberis | GCTAGTGATTGCAGCTGTGT | GCUAGUGAUUGCAGCUGUGU |
| | (SEQ ID NO: 152) | (SEQ ID NO: 187) |
| Streptococcus uberis | CAATGCATGGGAATGAAGGG | CAAUGCAUGGGAAUGAAGGG |
| | (SEQ ID NO: 153) | (SEQ ID NO: 188) |
| Streptococcus uberis | TTCCCAATCAACTTGCTAGG | UUCCCAAUCAACUUGCUAGG |
| | (SEQ ID NO: 154) | (SEQ ID NO: 189) |
| Streptococcus uberis | GCTTGAGGTTGTCCATGTTT | GCUUGAGGUUGUCCAUGUUU |
| | (SEQ ID NO: 155) | (SEQ ID NO: 190) |
| Streptococcus uberis | AAGCAAGAAGAGAGCCCCAG | AAGCAAGAAGAGAGCCCCAG |
| | (SEQ ID NO: 156) | (SEQ ID NO: 191) |
| Streptococcus uberis | TTCAGGCACATGGATCGAAT | UUCAGGCACAUGGAUCGAAU |
| | (SEQ ID NO: 157) | (SEQ ID NO: 192) |

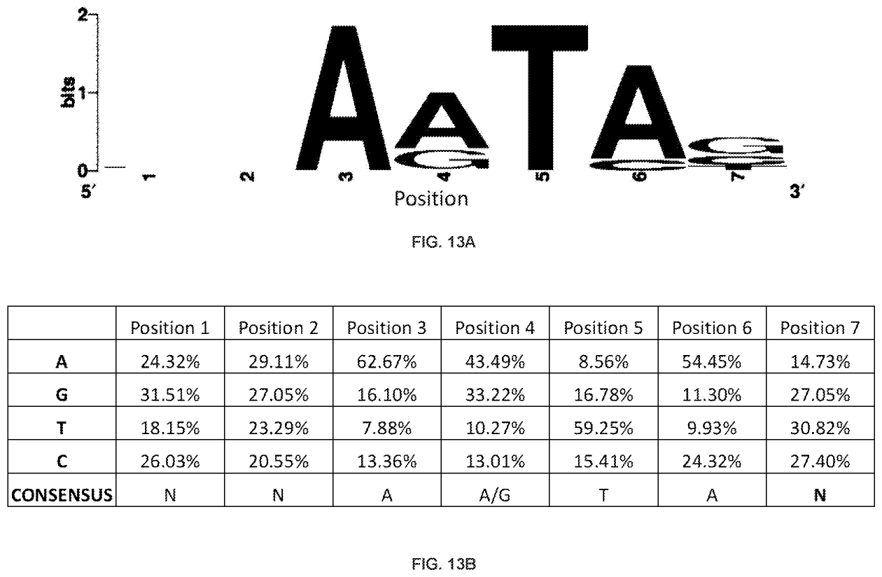

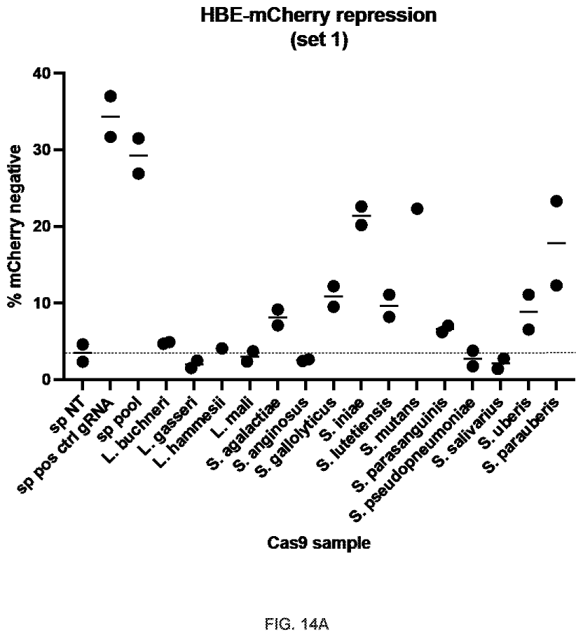

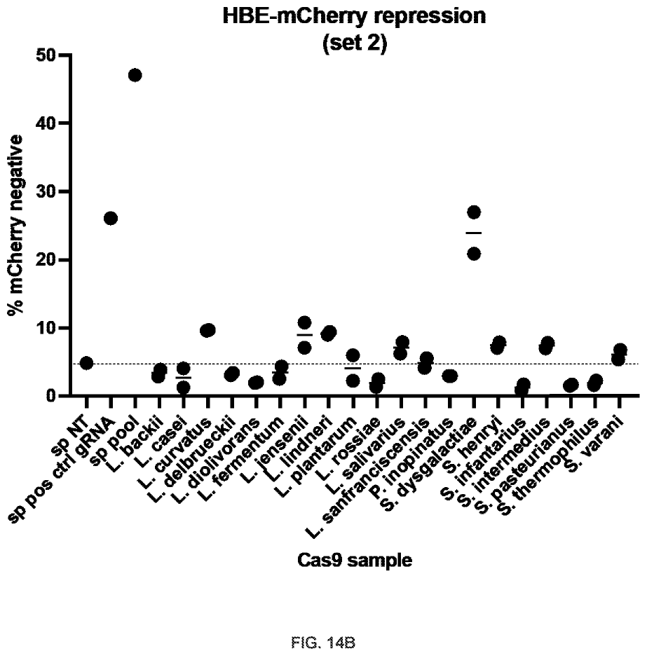

The flow cytometry results are shown in FIG. 14A-14B. The assay was done in two sets, with a different group of Cas9 proteins from various species in each set. Each set included Streptococcus pyogenes sp-dCas9-KRAB with HBE enhancer gRNA (“sp pos ctrl gRNA”) as a positive control, Streptococcus pyogenes sp-dCas9-KRAB with a pool of gRNAs targeting the HBE TSS (“sp pool”) as a positive control, and a negative control with Streptococcus pyogenes sp-dCas9-KRAB and a non-targeting gRNA (“sp NT”). In FIGS. 14A-14B, higher “percentage mCherry negative” indicated more effective repression, and data points above the dashed line indicated mCherry repression above background signal. The dCas9 effectors that lead to at least double the level of downregulation as the Streptococcus pyogenes Cas9 (Sp-dCas9) non-targeting control (Sp_NT) were considered as dCas9 sequences that are functional in mammalian cells. Based on these results, dCas9 from S. dysgalactiae, S. agalactiae, S. gallolyticus, S. iniae, S. lutetiensis, S. mutans, S. parauberis, and S. uberis showed excellent gene repression and were chosen for follow-up studies.

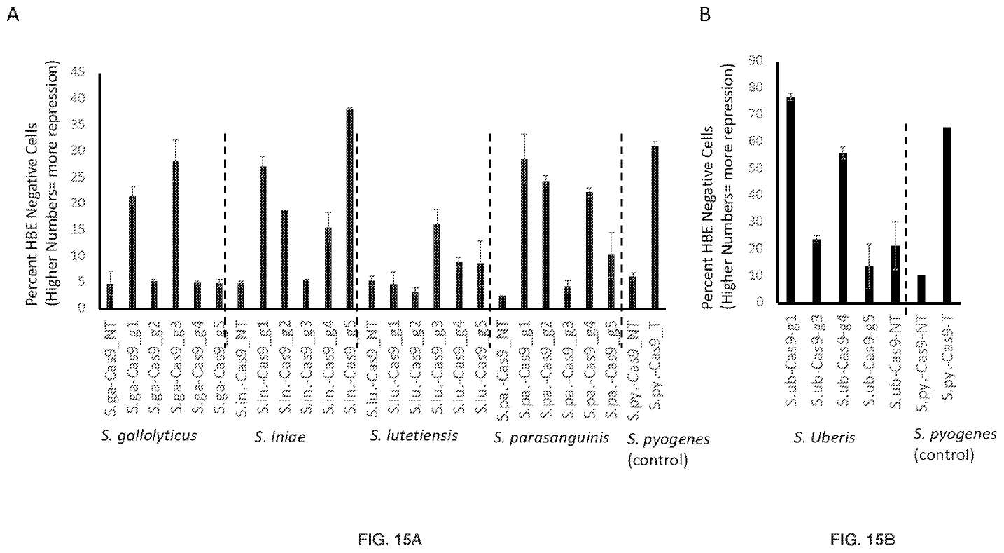

dCas9-KRAB fusion proteins with dCas9 from S. gallolyticus, S. iniae, S. parasanguinis, S. lutetiensis, and S. uberis were each studied further with individual gRNAs. K562 cells harboring an mCherry fluorescent tag on the HBE gene were transduced with lentiviruses encoding the dCas9-KRAB and a sgRNA targeting the HBE promoter or a non-targeting negative control. The gRNAs used are shown in TABLE 7. The cells were assayed for mCherry fluorescence 10 days later. S. pyogenes dCas9-KRAB was used as a positive control. Shown in FIG. 15A are results from S. gallolyticus dCas9-KRAB, S. iniae dCas9-KRAB, S. parasanguinis dCas9-KRAB, and S. lutetiensis dCas9-KRAB assayed in parallel with S. pyogenes dCas9-KRAB. Shown in FIG. 15B are results from S. uberis dCas9-KRAB assayed in parallel with S. pyogenes dCas9-KRAB. All dCas9-KRAB fusion proteins tested showed repression above the level of their corresponding non-targeting control with some sgRNA spacers, demonstrating that they function as repressors of gene expression in mammalian cells. The numbers in FIGS. 15A-15B denote HBE negative cells, and thus higher numbers meant more repressor activity.

TABLE 7
Spacer sequences (for gRNAs) targeting HBE for the dCas9
systems validated in the individual guide validations. Each
spacer sequence was cloned into the corresponding sgRNA scaffold.

| | | | | Target |
|---|---|---|---|---|
| Cas9 | gRNA | Predicted | | coordinate |
| Species | # | PAM | Spacer | on chr11 |
| Streptococcus | 1 | NNGTAAA | ACAGGGGGCCAGAACTTCGG | 5269976 |
| gallolyticus | | (SEQ ID | (SEQ ID NO: 283) | |
| | 2 | NO: 276) | CAAAAAATCTCTGGGTCCAG | 5269639 |
| | | | (SEQ ID NO: 284) | |
| | 3 | | GACGGCAGCCTTCTCCTCAG | 5269855 |
| | | | (SEQ ID NO: 285) | |
| | 4 | | TAGCTCTCTTAAGGAGTGCA | 5270395 |
| | | | (SEQ ID NO: 286) | |
| | 5 | | CATGGATCGAATTGAATACA | 5270917 |
| | | | (SEQ ID NO: 287) | |
| Streptococcus | 1 | NGG | GACTCCTCGTTGTTTACCCC | 5269655 |
| iniae | | (SEQ ID | (SEQ ID NO: 288) | |
| | 2 | NO: 2) | ATTACCCTAGCAAGTTGATT | 5269736 |
| | | | (SEQ ID NO: 289) | |
| | 3 | | TATTACCCTAGCAAGTTGAT | 5269737 |
| | | | (SEQ ID NO: 290) | |
| | 4 | | CTTCGGCAGTAAAGAATAAA | 5269966 |
| | | | (SEQ ID NO: 291) | |
| | 5 | | GCTAGTGATTGCAGCTGTGT | 5269911 |
| | | | (SEQ ID NO: 292) | |
| Streptococcus | 1 | NNAAAAA | TGCAGATAGATGAGGAGCCA | 5270135 |
| lutetiensis | | (SEQ ID | (SEQ ID NO: 293) | |
| | 2 | NO: 279) | GCAGATAGATGAGGAGCCAA | 5270134 |
| | | | (SEQ ID NO: 294) | |
| | 3 | | CGACAGGTTTCCAAAGCTGT | 5269619 |
| | | | (SEQ ID NO: 295) | |
| | 4 | | TCAGATACAAAATTAGAGAT | 5269695 |
| | | | (SEQ ID NO: 296) | |
| | 5 | | CAGATACAAAATTAGAGATG | 5269696 |
| | | | (SEQ ID NO: 297) | |
| Streptococcus | 1 | NNAAAG | TTACCCTAGCAAGTTGATTG | 5269732 |
| parasanguinis | | (SEQ ID | (SEQ ID NO: 298) | |
| | 2 | NO: 282) | CCCCTGTTCTCCATGGTACT | 5269996 |
| | | | (SEQ ID NO: 299) | |
| | 3 | | AGGGGGCCAGAACTTCGGCA | 5269975 |
| | | | (SEQ ID NO: 300) | |
| | 4 | | AGATAGATGAGGAGCCAACA | 5270133 |
| | | | (SEQ ID NO: 301) | |
| | 5 | | AGAACTTCGGCAGTAAAGAA | 5269967 |
| | | | (SEQ ID NO: 302) | |
| Streptococcus | 1 | NNAATA | CAATGCATGGGAATGAAGGG | 5269758 |
| uberis | | (SEQ ID | (SEQ ID NO: 303) | |
| | 2 | NO: 274) | TTCCCAATCAACTTGCTAGG | 5269734 |
| | | | (SEQ ID NO: 304) | |
| | 3 | | GCTTGAGGTTGTCCATGTTT | 5269519 |
| | | | (SEQ ID NO: 305) | |
| | 4 | | AAGCAAGAAGAGAGCCCCAG | 5270658 |
| | | | (SEQ ID NO: 306) | |
| | 5 | | TTCAGGCACATGGATCGAAT | 5270926 |
| | | | (SEQ ID NO: 307) | |

### Example 10

### S. gallolyticus Cas9 and S. iniae Cas9 Nuclease Activity

The nuclease activity of S. gallolyticus Cas9 and S. iniae Cas9 were tested in mammalian cells as described in Example 1. Guide RNAs from HBE repression experiments (Example 10) were used to target nuclease competent proteins to generate genomic insertions and deletions. Plasmids encoding each guide RNA were transfected into 293T cells along with a plasmid encoding nuclease competent S. gallolyticus Cas9 or S. iniae Cas9 protein. Results are shown in FIG. 16. An increase in insertions and deletions in the targeting gRNAs relative to non-targeting gRNAs indicated that these Cas9 proteins were effective nucleases in mammalian cells. Sequences of the gRNAs used are shown in TABLE 6.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A Clustered Regularly Interspaced Short Palindromic Repeats associated (Cas) protein comprising an amino acid sequence having at least 80%, 85%, 90%, 95%, or 98% or greater identity to at least one of SEQ ID NOs: 57, 241, 243, 245, 247, 249, 251, 235, or 223, or any fragment thereof, or wherein the Cas protein is from Streptococcus uberis, Streptococcus agalactiae, Streptococcus gallolyticus, Streptococcus iniae, Streptococcus lutetiensis, Streptococcus mutans, Streptococcus parauberis, Streptococcus dysgalactiae, or Streptococcus parasanguinis.

Clause 2. The Cas protein of clause 1, wherein the Cas protein comprises an amino acid sequence having one, two, three, four, five or more changes selected from amino acid substitutions, insertions, or deletions, relative to SEQ ID NO: 57, or any fragment thereof, or wherein the Cas protein comprises the amino acid sequence of SEQ ID NO: 57, or wherein the Cas protein is encoded by a polynucleotide comprising a sequence having at least 80%, 85%, 90%, 95%, or 98% or greater identity to SEQ ID NO: 58, or any fragment thereof, or wherein the Cas protein is encoded by a polynucleotide comprising a sequence having one, two, three, four, five or more changes selected from nucleotide substitutions, insertions, or deletions, relative to SEQ ID NO: 58, or any fragment thereof, or wherein the Cas protein is encoded by a polynucleotide comprising the sequence of SEQ ID NO: 58.

Clause 3. The Cas protein of clause 1, wherein the Cas protein comprises an amino acid sequence having one, two, three, four, five or more changes selected from amino acid substitutions, insertions, or deletions, relative to SEQ ID NO: 223, or any fragment thereof, or wherein the Cas protein comprises the amino acid sequence of SEQ ID NO: 223, or wherein the Cas protein is encoded by a polynucleotide comprising a sequence having at least 80%, 85%, 90%, 95%, or 98% or greater identity to SEQ ID NO: 224, or any fragment thereof, or wherein the Cas protein is encoded by a polynucleotide comprising a sequence having one, two, three, four, five or more changes selected from nucleotide substitutions, insertions, or deletions, relative to SEQ ID NO: 224, or any fragment thereof, or wherein the Cas protein is encoded by a polynucleotide comprising the sequence of SEQ ID NO: 224.

Clause 4. The Cas protein of clause 1, wherein the Cas protein comprises an amino acid sequence having one, two, three, four, five or more changes selected from amino acid substitutions, insertions, or deletions, relative to SEQ ID NO: 241, or any fragment thereof, or wherein the Cas protein comprises the amino acid sequence of SEQ ID NO: 241, or wherein the Cas protein is encoded by a polynucleotide comprising a sequence having at least 80%, 85%, 90%, 95%, or 98% or greater identity to SEQ ID NO: 242, or any fragment thereof, or wherein the Cas protein is encoded by a polynucleotide comprising a sequence having one, two, three, four, five or more changes selected from nucleotide substitutions, insertions, or deletions, relative to SEQ ID NO: 242, or any fragment thereof, or wherein the Cas protein is encoded by a polynucleotide comprising the sequence of SEQ ID NO: 242.

Clause 5. The Cas protein of clause 1, wherein the Cas protein comprises an amino acid sequence having one, two, three, four, five or more changes selected from amino acid substitutions, insertions, or deletions, relative to SEQ ID NO: 243, or any fragment thereof, or wherein the Cas protein comprises the amino acid sequence of SEQ ID NO: 243, or wherein the Cas protein is encoded by a polynucleotide comprising a sequence having at least 80%, 85%, 90%, 95%, or 98% or greater identity to SEQ ID NO: 244, or any fragment thereof, or wherein the Cas protein is encoded by a polynucleotide comprising a sequence having one, two, three, four, five or more changes selected from nucleotide substitutions, insertions, or deletions, relative to SEQ ID NO: 244, or any fragment thereof, or wherein the Cas protein is encoded by a polynucleotide comprising the sequence of SEQ ID NO: 244.

Clause 6. The Cas protein of clause 1, wherein the Cas protein comprises an amino acid sequence having one, two, three, four, five or more changes selected from amino acid substitutions, insertions, or deletions, relative to SEQ ID NO: 245, or any fragment thereof, or wherein the Cas protein comprises the amino acid sequence of SEQ ID NO: 245, or wherein the Cas protein is encoded by a polynucleotide comprising a sequence having at least 80%, 85%, 90%, 95%, or 98% or greater identity to SEQ ID NO: 246, or any fragment thereof, or wherein the Cas protein is encoded by a polynucleotide comprising a sequence having one, two, three, four, five or more changes selected from nucleotide substitutions, insertions, or deletions, relative to SEQ ID NO: 246, or any fragment thereof, or wherein the Cas protein is encoded by a polynucleotide comprising the sequence of SEQ ID NO: 246.

Clause 7. The Cas protein of clause 1, wherein the Cas protein comprises an amino acid sequence having one, two, three, four, five or more changes selected from amino acid substitutions, insertions, or deletions, relative to SEQ ID NO: 247, or any fragment thereof, or wherein the Cas protein comprises the amino acid sequence of SEQ ID NO: 247, or wherein the Cas protein is encoded by a polynucleotide comprising a sequence having at least 80%, 85%, 90%, 95%, or 98% or greater identity to SEQ ID NO: 248, or any fragment thereof, or wherein the Cas protein is encoded by a polynucleotide comprising a sequence having one, two, three, four, five or more changes selected from nucleotide substitutions, insertions, or deletions, relative to SEQ ID NO: 248, or any fragment thereof, or wherein the Cas protein is encoded by a polynucleotide comprising the sequence of SEQ ID NO: 248.

Clause 8. The Cas protein of clause 1, wherein the Cas protein comprises an amino acid sequence having one, two, three, four, five or more changes selected from amino acid substitutions, insertions, or deletions, relative to SEQ ID NO: 249, or any fragment thereof, or wherein the Cas protein comprises the amino acid sequence of SEQ ID NO: 249, or wherein the Cas protein is encoded by a polynucleotide comprising a sequence having at least 80%, 85%, 90%, 95%, or 98% or greater identity to SEQ ID NO: 250, or any fragment thereof, or wherein the Cas protein is encoded by a polynucleotide comprising a sequence having one, two, three, four, five or more changes selected from nucleotide substitutions, insertions, or deletions, relative to SEQ ID NO: 250, or any fragment thereof, or wherein the Cas protein is encoded by a polynucleotide comprising the sequence of SEQ ID NO: 250.

Clause 9. The Cas protein of clause 1, wherein the Cas protein comprises an amino acid sequence having one, two, three, four, five or more changes selected from amino acid substitutions, insertions, or deletions, relative to SEQ ID NO: 251, or any fragment thereof, or wherein the Cas protein comprises the amino acid sequence of SEQ ID NO: 251, or wherein the Cas protein is encoded by a polynucleotide comprising a sequence having at least 80%, 85%, 90%, 95%, or 98% or greater identity to SEQ ID NO: 252, or any fragment thereof, or wherein the Cas protein is encoded by a polynucleotide comprising a sequence having one, two, three, four, five or more changes selected from nucleotide substitutions, insertions, or deletions, relative to SEQ ID NO: 252, or any fragment thereof, or wherein the Cas protein is encoded by a polynucleotide comprising the sequence of SEQ ID NO: 252.

Clause 10. The Cas protein of clause 1, wherein the Cas protein comprises an amino acid sequence having one, two, three, four, five or more changes selected from amino acid substitutions, insertions, or deletions, relative to SEQ ID NO: 235, or any fragment thereof, or wherein the Cas protein comprises the amino acid sequence of SEQ ID NO: 235, or wherein the Cas protein is encoded by a polynucleotide comprising a sequence having at least 80%, 85%, 90%, 95%, or 98% or greater identity to SEQ ID NO: 236, or any fragment thereof, or wherein the Cas protein is encoded by a polynucleotide comprising a sequence having one, two, three, four, five or more changes selected from nucleotide substitutions, insertions, or deletions, relative to SEQ ID NO: 236, or any fragment thereof, or wherein the Cas protein is encoded by a polynucleotide comprising the sequence of SEQ ID NO: 236.

Clause 11. The Cas protein of clause any one of clauses 1-10, wherein the Cas protein comprises at least one amino acid mutation that knocks out nuclease activity of the Cas protein.

Clause 12. The Cas protein of clause 11, wherein the at least one amino acid mutation is at least one of D10A, H600A, H845A, H599A, H840A, H604A, H839A, and D9A.

Clause 13. The Cas protein of any one of clauses 11-12, wherein the Cas protein comprises an amino acid sequence having at least 80%, 85%, 90%, 95%, or 98% or greater identity to at least one of SEQ ID NOs: 59, 193, 197, 201, 205, 209, 213, 237, 225, or any fragment thereof.

Clause 14. The Cas protein of clause 13, wherein the Cas protein comprises an amino acid sequence having one, two, three, four, five or more changes selected from amino acid substitutions, insertions, or deletions, relative to at least one of SEQ ID NOs: 59, 193, 197, 201, 205, 209, 213, 237, 225, or any fragment thereof.

Clause 15. The Cas protein of clause 13 or 14, wherein the Cas protein comprises the amino acid sequence of at least one of SEQ ID NOs: 59, 193, 197, 201, 205, 209, 213, 237, or 225.

Clause 16. The Cas protein of any one of clauses 11-15, wherein the Cas protein is encoded by a polynucleotide comprising a sequence having at least 80%, 85%, 90%, 95%, or 98% or greater identity to at least one of SEQ ID NOs: 60, 194, 198, 202, 206, 210, 214, 238, 226, or any fragment thereof.

Clause 17. The Cas protein of clause 16, wherein the Cas protein is encoded by a polynucleotide comprising a sequence having one, two, three, four, five or more changes selected from nucleotide substitutions, insertions, or deletions, relative to at least one of SEQ ID NOs: 60, 194, 198, 202, 206, 210, 214, 238, 226, or any fragment thereof.

Clause 18. The Cas protein of clause 16 or 17, wherein the Cas protein is encoded by a polynucleotide comprising the sequence of at least one of SEQ ID NOs: 60, 194, 198, 202, 206, 210, 214, 238, or 226.

Clause 19. The Cas protein of any one of clauses 1-18, wherein the Cas protein recognizes a PAM sequence of AATA (SEQ ID NO: 71), NNA(A/G)TAN (SEQ ID NO: 273), NNAATA (SEQ ID NO: 274), NNG(T/C)(G/A)AN (SEQ ID NO: 275), NNGTAAA (SEQ ID NO: 276), NNGGNNN (SEQ ID NO: 277), NGG (SEQ ID NO: 2), NNAAAAN (SEQ ID NO: 278), NNAAAAA (SEQ ID NO: 279), NNGGNTN (SEQ ID NO: 280), NNAA(A/G)GN (SEQ ID NO: 281), and/or NNAAAG (SEQ ID NO: 282).

Clause 20. A fusion protein comprising two heterologous polypeptide domains, wherein the first polypeptide domain comprises the Cas protein of any one of clauses 1-19, and wherein the second polypeptide domain has an activity selected from the group consisting of transcription activation activity, transcription repression activity, transcription release factor activity, histone modification activity, nuclease activity, nucleic acid association activity, methylase activity, and demethylase activity, or a combination thereof.

Clause 21. The fusion protein of clause 20, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4× repressor, Mxil repressor, SUV39H1, SUV39H2, G9A, ESET/SETBD1, Cir4, Su (var) 3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

Clause 22. The fusion protein of any one of clauses 20-21, wherein the second polypeptide domain has transcription repression activity.

Clause 23. The fusion protein of clause 22, wherein the second polypeptide domain comprises KRAB.

Clause 24. The fusion protein of clause 23, wherein the KRAB comprises an amino acid sequence having at least 80%, 85%, 90%, 95%, or 98% or greater identity to SEQ ID NO: 45, or comprises an amino acid sequence having one, two, three, four, five or more changes selected from amino acid substitutions, insertions, or deletions, relative to SEQ ID NO: 45, or comprises the amino acid sequence of SEQ ID NO: 45, or is encoded by a polynucleotide comprising a sequence having at least 80%, 85%, 90%, 95%, or 98% or greater identity to SEQ ID NO: 46, or is encoded by a polynucleotide comprising a sequence having one, two, three, four, five or more changes selected from nucleotide substitutions, insertions, or deletions, relative to SEQ ID NO: 46 or is encoded by a polynucleotide comprising the sequence of SEQ ID NO: 46, or any fragment thereof.

Clause 25. The fusion protein of any one of clauses 20-24, wherein the fusion protein comprises an amino acid sequence having at least 80%, 85%, 90%, 95%, or 98% or greater identity to at least one of SEQ ID NOs: 61, 217, 218, 219, 220, 221, 222, 239, 227, or comprises an amino acid sequence having one, two, three, four, five or more changes selected from amino acid substitutions, insertions, or deletions, relative to at least one of SEQ ID NOs: 61, 217, 218, 219, 220, 221, 222, 239, 227, or comprises the amino acid sequence of at least one of SEQ ID NOs: 61, 217, 218, 219, 220, 221, 222, 239, 227, or is encoded by a polynucleotide comprising a sequence having at least 80%, 85%, 90%, 95%, or 98% or greater identity to SEQ ID NO: 62 or 240 or 228, or is encoded by a polynucleotide comprising a sequence having one, two, three, four, five or more changes selected from nucleotide substitutions, insertions, or deletions, relative to SEQ ID NO: 62 or 240 or 228, or is encoded by a polynucleotide comprising the sequence of SEQ ID NO: 62 or 240 or 228, or any fragment thereof.

Clause 26. The fusion protein of any one of clauses 20-21, wherein the second polypeptide domain has transcription activation activity.

Clause 27. The fusion protein of clause 26, wherein the second polypeptide domain comprises p300 or a fragment thereof or VP64 or a fragment thereof.

Clause 28. The fusion protein of clause 27, wherein the p300 or a fragment thereof comprises an amino acid sequence having at least 80%, 85%, 90%, 95%, or 98% or greater identity to SEQ ID NO: 41 or 42, or comprises an amino acid sequence having one, two, three, four, five or more changes selected from amino acid substitutions, insertions, or deletions, relative to SEQ ID NO: 41 or 42, or comprises the amino acid sequence of SEQ ID NO: 41 or 42, or any fragment thereof.

Clause 29. The fusion protein of any one of clauses 20-24, wherein the fusion protein comprises an amino acid sequence having at least 80%, 85%, 90%, 95%, or 98% or greater identity to at least one of SEQ ID NOs: 253, 259, 263, 265, 267, 261, 269, 271, or 229, or comprises an amino acid sequence having one, two, three, four, five or more changes selected from amino acid substitutions, insertions, or deletions, relative to at least one of SEQ ID NOs: 253, 259, 263, 265, 267, 261, 269, 271, or 229, or comprises the amino acid sequence of at least one of SEQ ID NOs: 253, 259, 263, 265, 267, 261, 269, 271, or 229, or is encoded by a polynucleotide comprising a sequence having at least 80%, 85%, 90%, 95%, or 98% or greater identity to at least one of SEQ ID NO: 254, 260, 264, 266, 268, 262, 270, 272, or 230, or is encoded by a polynucleotide comprising a sequence having one, two, three, four, five or more changes selected from nucleotide substitutions, insertions, or deletions, relative to at least one of SEQ ID NO: 254, 260, 264, 266, 268, 262, 270, 272, or 230, or is encoded by a polynucleotide comprising the sequence of at least one of SEQ ID NO: 254, 260, 264, 266, 268, 262, 270, 272, or 230, or any fragment thereof.

Clause 30. A DNA targeting composition comprising: the Cas protein of any one of clauses 1-19 or the fusion protein of any one of clauses 20-29; and at least one guide RNA (gRNA) that targets the Cas protein to a target region of a target gene.

Clause 31. The DNA targeting composition of clause 30, wherein the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene.

Clause 32. The DNA targeting composition of clause 31, wherein the gRNA targets the Cas protein to a promoter of the target gene.

Clause 33. The DNA targeting composition of clause 31, wherein the target region is located between about 1 to about 1000 base pairs upstream of a transcription start site of the target gene.

Clause 34. The DNA targeting composition of any one of clauses 30-33, wherein the DNA targeting composition comprises two or more gRNAs, each gRNA binding to a different target region.

Clause 35. The DNA targeting composition of any one of clauses 30-34, wherein the at least one gRNA comprises the sequence of SEQ ID NO: 69 or 67 or is encoded by or targets a sequence comprising SEQ ID NO: 70 or 68.

Clause 36. The DNA targeting composition of any one of clauses 30-34, wherein the at least one gRNA comprises a sequence selected from SEQ ID NOs: 195, 199, 203, 207, 211, 215, or is encoded by or targets a polynucleotide comprising a sequence selected from SEQ ID NOs: 196, 200, 204, 208, 212, 216.

Clause 37. The DNA targeting composition of any one of clauses 30-36, wherein the at least one gRNA comprises a sequence selected from SEQ ID NOs: 91-94, 100-103, 108-122, 158-192, or is encoded by or targets a polynucleotide comprising a sequence selected from SEQ ID NOs: 76-90, 96-99, 123-157.

Clause 38. An isolated polynucleotide sequence encoding the Cas protein of any one of clauses 1-19 or the fusion protein of any one of clauses 20-29, or the DNA targeting composition of any one of clauses 31-38.

Clause 39. A vector comprising: the isolated polynucleotide sequence of clause 38.

Clause 40. The vector of clause 39, wherein the vector is an adeno-associated virus (AAV) vector.

Clause 41. A cell comprising: the DNA targeting composition of any one of clauses 30-37, or the isolated polynucleotide sequence of clause 38, or the vector of clause 39 or 40, or a combination thereof.

Clause 42. A pharmaceutical composition comprising: the DNA targeting composition of any one of clauses 30-37, or the isolated polynucleotide sequence of clause 38, or the vector of clause 39 or 40, or a combination thereof.

Clause 43. A method of modulating expression of a gene in a cell or in a subject, the method comprising administering to the cell or the subject the DNA targeting composition of any one of clauses 30-37, or the isolated polynucleotide sequence of clause 38, or the vector of clause 39 or 40, or the pharmaceutical composition of clause 42, or a combination thereof.

Clause 44. The method of clause 43, wherein the expression of the gene is increased relative to a control.

Clause 45. The method of clause 43, wherein the expression of the gene is decreased relative to a control.

Clause 46. The method of clause 43, wherein the gene comprises the dystrophin gene.

Clause 47. A method of correcting a mutant gene in a cell, the method comprising administering to the cell or the subject the DNA targeting composition of any one of clauses 30-37, or the isolated polynucleotide sequence of clause 38, or the vector of clause 39 or 40, or the pharmaceutical composition of clause 42, or a combination thereof.

Clause 48. The method of clause 47, further comprising administering to the cell or subject a donor DNA.

Clause 49. The method of clause 47 or 48, wherein correcting a mutant gene comprises deleting, rearranging, or replacing the mutant gene.

Clause 50. The method of any one of clauses 7-49, wherein the gene comprises the dystrophin gene.

Clause 51. A method of treating a disease in a subject, the method comprising administering to the subject the DNA targeting composition of any one of clauses 30-37, or the isolated polynucleotide sequence of clause 38, or the vector of clause 39 or 40, or the cell of clause 41, or the pharmaceutical composition of clause 42, or a combination thereof.

Clause 52. The method of clause 51, wherein the DNA targeting composition, or the isolated polynucleotide sequence, or the vector, or the cell, or the pharmaceutical composition, or a combination thereof, is administered to skeletal muscle or cardiac muscle of the subject.

Clause 53. The method of clause 51 or 52, wherein the disease comprises Duchenne muscular dystrophy (DMD) or Becker muscular dystrophy (BMD).

SEQUENCES
SEQ ID NO: 1
NRG
(R = A or G; N can be any nucleotide residue, e.g., any of A, G, C, or T)
SEQ ID NO: 2
NGG
(N can be any nucleotide residue, e.g., any of A, G, C, or T)
SEQ ID NO: 3
NAG
(N can be any nucleotide residue, e.g., any of A, G, C, or T)
SEQ ID NO: 4
NGGNG
(N can be any nucleotide residue, e.g., any of A, G, C, or T)
SEQ ID NO: 5
NNAGAAW
(W = A or T; N can be any nucleotide residue, e.g., any of A, G, C, or T)
SEQ ID NO: 6
NAAR
(R = A or G; N can be any nucleotide residue, e.g., any of A, G, C, or T)
SEQ ID NO: 7
NNGRR
(R = A or G; N can be any nucleotide residue, e.g., any of A, G, C, or T)
SEQ ID NO: 8
NNGRRN
(R = A or G; N can be any nucleotide residue, e.g., any of A, G, C, or T)
SEQ ID NO: 9
NNGRRT
(R = A or G; N can be any nucleotide residue, e.g., any of A, G, C, or T)
SEQ ID NO: 10
NNGRRV
(R = A or G; N can be any nucleotide residue, e.g., any of A, G, C, or T; V = A or
C or G)
SEQ ID NO: 11
NNNNGATT
(N can be any nucleotide residue, e.g., any of A, G, C, or T)
SEQ ID NO: 12
NNNNGNNN
(N can be any nucleotide residue, e.g., any of A, G, C, or T)
SEQ ID NO: 13
NGA
(N can be any nucleotide residue, e.g., any of A, G, C, or T)
SEQ ID NO: 14
NNNRRT
(R = A or G; N can be any nucleotide residue, e.g., any of A, G, C, or T)
SEQ ID NO: 15
ATTCCT
SEQ ID NO: 16
NGAN
(N can be any nucleotide residue, e.g., any of A, G, C, or T)
SEQ ID NO: 17
NGNG
(N can be any nucleotide residue, e.g., any of A, G, C, or T)
DNA sequence of the gRNA constant region for spCas9
SEQ ID NO: 18
gtttaagagctatgctggaaacagcatagcaagtttaaataaggctagtccgttatcaacttgaaaaa
gtggcaccgagtcggtgc
RNA sequence of the gRNA constant region for spCas9
SEQ ID NO: 19
guuuaagagcuaugcuggaaacagcauagcaaguuuaaauaaggcuaguccguuaucaacuugaaaaa
guggcaccgagucggugc
SV40 NLS
SEQ ID NO: 20
(Pro-Lys-Lys-Lys-Arg-Lys-Val)
GS linker
SEQ ID NO: 21
(Gly-Gly-Gly-Gly-Ser)ₙ,
wherein n is an integer between 0 and 10
SEQ ID NO: 22
Gly-Gly-Gly-Gly-Gly
SEQ ID NO: 23
Gly-Gly-Ala-Gly-Gly
SEQ ID NO: 24
Gly-Gly-Gly-Gly-Ser-Ser-Ser
SEQ ID NO: 25
Gly-Gly-Gly-Gly-Ala-Ala-Ala
Streptococcus pyogenes Cas9
SEQ ID NO: 26
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLEGNLIALSLGLTPNEKSNEDLAEDAKLQLSKDTYD
DDLDNLLAQIGDQYADLELAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTEDNG
SIPHQIHLGELHAILRRQEDFYPFLKDNREKIEKILTFRIPYYVGPLARGNSRFAWMTRKSEETITPW
NFEEVVDKGASAQSFIERMTNEDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLEDDKVMKQLKRRRYTGWGRLSRKLINGIRDKQSGKTIL
DFLKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYEDTTIDRKRYTSTKEVLDATLIHQSITGLYETRI
DLSQLGGD
Staphylococcus aureus Cas9
SEQ ID NO: 27
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSEDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
Streptococcus pyogenes Cas9 (with D10A)
SEQ ID NO: 28
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNEKSNEDLAEDAKLQLSKDTYD
DDLDNLLAQIGDQYADLELAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTEDNG
SIPHQIHLGELHAILRRQEDFYPELKDNREKIEKILTFRIPYYVGPLARGNSRFAWMTRKSEETITPW
NFEEVVDKGASAQSFIERMTNEDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLEDDKVMKQLKRRRYTGWGRLSRKLINGIRDKQSGKTIL
DFLKSDGFANRNEMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Streptococcus pyogenes Cas9 (with D10A, H849A)
SEQ ID NO: 29
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNEKSNEDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTEDNG
SIPHQIHLGELHAILRRQEDFYPELKDNREKIEKILTFRIPYYVGPLARGNSRFAWMTRKSEETITPW
NFEEVVDKGASAQSFIERMTNEDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLEDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKEDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
sequence of mutant S. aureus 10 Cas9
SEQ ID NO: 30
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
Polynucleotide sequence of N580A mutant of S. aureus 10 Cas9
SEQ ID NO: 31
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized polynucleotide encoding S. pyogenes 10 Cas9
SEQ ID NO: 32
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga aacacggatc
gacctctctc aactgggcgg cgactag
codon optimized nucleic acid sequences encoding S. aureus 10 Cas9
SEQ ID NO: 33
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca totttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
codon optimized nucleic acid sequences encoding S. aureus 10 Cas9
SEQ ID NO: 34
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tottcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
codon optimized nucleic acid sequence encoding S. aureus 10 Cas9
SEQ ID NO: 35
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tottcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcato aagaaggga
codon optimized nucleic acid sequence encoding S. aureus 10 Cas9
SEQ ID NO: 36
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggggccacgaaaaaggccggccaggcaaaaaagaaaaag
codon optimized nucleic acid sequence encoding S. aureus 10 Cas9
SEQ ID NO: 37
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
codon optimized nucleic acid sequences encoding S. aureus 10 Cas9
SEQ ID NO: 38
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
codon optimized nucleic acid sequences encoding S. aureus 10 Cas9
SEQ ID NO: 39
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
Vector (pDO242) encoding codon optimized nucleic acid sequence
encoding S. aureus 10 Cas9
SEQ ID NO: 40
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
Human p300 (with L553M mutation) protein
SEQ ID NO: 41
MAENVVEPGPPSAKRPKLSSPALSASASDGTDFGSLEDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKESAKR
LPSTRLGTFLENRVNDELRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
Human p300 Core Effector protein (aa 1048-1664 of SEQ ID NO: 41)
SEQ ID NO: 42
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDELRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDEWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
VP64-dCas9-VP64 protein
SEQ ID NO: 43
RADALDDFDLDMLGSDALDDFDLDMLGSDALDDFDLDMLGSDALDDEDLDMVNPKKKRKVGRGMDKKY
SIGLAIGTNSVGWAVITDEYKVPSKKFKVLGNTDRHSIKKNLIGALLEDSGETAEATRLKRTARRRYT
RRKNRICYLQEIFSNEMAKVDDSFFHRLEESELVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNEDLAEDAKLQLSKDTYDDDLDN
LLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTEDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRENASLGTYHDLLKIIKDKDELDNEENEDILE
DIVLTLTLFEDREMIEERLKTYAHLEDDKVMKQLKRRRYTGWGRLSRKLINGIRDKQSGKTILDELKS
DGFANRNEMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKEDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGEDSPTVAYSVLVVAKVEKGKSKKLKSVK
ELLGITIMERSSFEKNPIDELEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDEDLDMLGSDALDDEDLDMLGSDALDDEDLDML
I
VP64-dCas9-VP64 DNA
SEQ ID NO: 44
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
Polypeptide sequence of KRAB protein
SEQ ID NO: 45
RTLVTFKDVFVDFTREEWKLLDTAQQILYRNVMLENYKNLVSLGYQLTKPDVILRLEKGEEP
WLV
Polynucleotide sequence for KRAB
SEQ ID NO: 46
cggacactggtgaccttcaaggatgtgtttgtggacttcaccagggaggagtggaagctgct
ggacactgctcagcagatcctgtacagaaatgtgatgctggagaactataagaacctggttt
ccttgggttatcagcttactaagccagatgtgatcctccggttggagaagggagaagagccc
tggctggtg
Polypeptide sequence of Streptococcus pyogenes dCas9-KRAB protein
SEQ ID NO: 47
MDYKDHDGDYKDHDIDYKDDDDKMAPKKKRKVGRGMDKKYSIGLAIGTNSVGWAVITDEYKVPSKKEK
VLGNTDRHSIKKNLIGALLFDSGETAEATRLKRTARRRYTRRKNRICYLQEIFSNEMAKVDDSFFHRL
EESELVEEDKKHERHPIFGNIVDEVAYHEKYPTIYHLRKKLVDSTDKADLRLIYLALAHMIKERGHEL
IEGDLNPDNSDVDKLFIQLVQTYNQLFEENPINASGVDAKAILSARLSKSRRLENLIAQLPGEKKNGL
FGNLIALSLGLTPNFKSNEDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGASQEEFY
KFIKPILEKMDGTEELLVKLNREDLLRKQRTEDNGSIPHQIHLGELHAILRRQEDFYPELKDNREKIE
KILTFRIPYYVGPLARGNSRFAWMTRKSEETITPWNFEEVVDKGASAQSFIERMTNFDKNLPNEKVLP
KHSLLYEYFTVYNELTKVKYVTEGMRKPAFLSGEQKKAIVDLLFKTNRKVTVKQLKEDYFKKIECFDS
VEISGVEDRENASLGTYHDLLKIIKDKDELDNEENEDILEDIVLTLTLFEDREMIEERLKTYAHLEDD
KVMKQLKRRRYTGWGRLSRKLINGIRDKQSGKTILDELKSDGFANRNFMQLIHDDSLTFKEDIQKAQV
SGQGDSLHEHIANLAGSPAIKKGILQTVKVVDELVKVMGRHKPENIVIEMARENQTTQKGQKNSRERM
KRIEEGIKELGSQILKEHPVENTQLQNEKLYLYYLQNGRDMYVDQELDINRLSDYDVDAIVPQSFLKD
DSIDNKVLTRSDKNRGKSDNVPSEEVVKKMKNYWRQLLNAKLITQRKEDNLTKAERGGLSELDKAGFI
KRQLVETRQITKHVAQILDSRMNTKYDENDKLIREVKVITLKSKLVSDERKDFQFYKVREINNYHHAH
DAYLNAVVGTALIKKYPKLESEFVYGDYKVYDVRKMIAKSEQEIGKATAKYFFYSNIMNFFKTEITLA
NGEIRKRPLIETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLTNLGAPAAFK
YFDTTIDRKRYTSTKEVLDATLIHQSITGLYETRIDLSQLGGDSRADPKKKRKVASDAKSLTAWSRTL
VTFKDVFVDFTREEWKLLDTAQQILYRNVMLENYKNLVSLGYQLTKPDVILRLEKGEEPWLVEREIHQ
ETHPDSETAFEIKSSVPKKKRKV
Polynucleotide sequence encoding Streptococcus pyogenes dCas9-KRAB
SEQ ID NO: 48
atggactacaaagaccatgacggtgattataaagatcatgacatcgattacaaggatgacgatgacaa
gatggcccccaagaagaagaggaaggtgggccgcggaatggacaagaagtactccattgggctcgcca
tcggcacaaacagcgtcggctgggccgtcattacggacgagtacaaggtgccgagcaaaaaattcaaa
gttctgggcaataccgatcgccacagcataaagaagaacctcattggcgccctcctgttcgactccgg
ggaaaccgccgaagccacgcggctcaaaagaacagcacggcgcagatatacccgcagaaagaatcgga
tctgctacctgcaggagatctttagtaatgagatggctaaggtggatgactctttcttccataggctg
gaggagtcctttttggtggaggaggataaaaagcacgagcgccacccaatctttggcaatatcgtgga
cgaggtggcgtaccatgaaaagtacccaaccatatatcatctgaggaagaagcttgtagacagtactg
ataaggctgacttgcggttgatctatctcgcgctggcgcatatgatcaaatttcggggacacttcctc
atcgagggggacctgaacccagacaacagcgatgtcgacaaactctttatccaactggttcagactta
caatcagcttttcgaagagaacccgatcaacgcatccggagttgacgccaaagcaatcctgagcgcta
ggctgtccaaatcccggcggctcgaaaacctcatcgcacagctccctggggagaagaagaacggcctg
tttggtaatcttatcgccctgtcactcgggctgacccccaactttaaatctaacttcgacctggccga
agatgccaagcttcaactgagcaaagacacctacgatgatgatctcgacaatctgctggcccagatcg
gcgaccagtacgcagacctttttttggcggcaaagaacctgtcagacgccattctgctgagtgatatt
ctgcgagtgaacacggagatcaccaaagctccgctgagcgctagtatgatcaagcgctatgatgagca
ccaccaagacttgactttgctgaaggcccttgtcagacagcaactgcctgagaagtacaaggaaattt
tcttcgatcagtctaaaaatggctacgccggatacattgacggcggagcaagccaggaggaattttac
aaatttattaagcccatcttggaaaaaatggacggcaccgaggagctgctggtaaagcttaacagaga
agatctgttgcgcaaacagcgcactttcgacaatggaagcatcccccaccagattcacctgggcgaac
tgcacgctatcctcaggcggcaagaggatttctacccctttttgaaagataacagggaaaagattgag
aaaatcctcacatttcggataccctactatgtaggccccctcgcccggggaaattccagattcgcgtg
gatgactcgcaaatcagaagagaccatcactccctggaacttcgaggaagtcgtggataagggggcct
ctgcccagtccttcatcgaaaggatgactaactttgataaaaatctgcctaacgaaaaggtgcttcct
aaacactctctgctgtacgagtacttcacagtttataacgagctcaccaaggtcaaatacgtcacaga
agggatgagaaagccagcattcctgtctggagagcagaagaaagctatcgtggacctcctcttcaaga
cgaaccggaaagttaccgtgaaacagctcaaagaagactatttcaaaaagattgaatgtttcgactct
gttgaaatcagcggagtggaggatcgcttcaacgcatccctgggaacgtatcacgatctcctgaaaat
cattaaagacaaggacttcctggacaatgaggagaacgaggacattcttgaggacattgtcctcaccc
ttacgttgtttgaagatagggagatgattgaagaacgcttgaaaacttacgctcatctcttcgacgac
aaagtcatgaaacagctcaagaggcgccgatatacaggatgggggcggctgtcaagaaaactgatcaa
tgggatccgagacaagcagagtggaaagacaatcctggattttcttaagtccgatggatttgccaacc
ggaacttcatgcagttgatccatgatgactctctcacctttaaggaggacatccagaaagcacaagtt
tctggccagggggacagtcttcacgagcacatcgctaatcttgcaggtagcccagctatcaaaaaggg
aatactgcagaccgttaaggtcgtggatgaactcgtcaaagtaatgggaaggcataagcccgagaata
tcgttatcgagatggcccgagagaaccaaactacccagaagggacagaagaacagtagggaaaggatg
aagaggattgaagagggtataaaagaactggggtcccaaatccttaaggaacacccagttgaaaacac
ccagcttcagaatgagaagctctacctgtactacctgcagaacggcagggacatgtacgtggatcagg
aactggacatcaatcggctctccgactacgacgtggatgccatcgtgccccagtcttttctcaaagat
gattctattgataataaagtgttgacaagatccgataaaaatagagggaagagtgataacgtcccctc
agaagaagttgtcaagaaaatgaaaaattattggcggcagctgctgaacgccaaactgatcacacaac
ggaagttcgataatctgactaaggctgaacgaggtggcctgtctgagttggataaagccggcttcatc
aaaaggcagcttgttgagacacgccagatcaccaagcacgtggcccaaattctcgattcacgcatgaa
caccaagtacgatgaaaatgacaaactgattcgagaggtgaaagttattactctgaagtctaagctgg
tctcagatttcagaaaggactttcagttttataaggtgagagagatcaacaattaccaccatgcgcat
gatgcctacctgaatgcagtggtaggcactgcacttatcaaaaaatatcccaagcttgaatctgaatt
tgtttacggagactataaagtgtacgatgttaggaaaatgatcgcaaagtctgagcaggaaataggca
aggccaccgctaagtacttcttttacagcaatattatgaattttttcaagaccgagattacactggcc
aatggagagattcggaagcgaccacttatcgaaacaaacggagaaacaggagaaatcgtgtgggacaa
gggtagggatttcgcgacagtccggaaggtcctgtccatgccgcaggtgaacatcgttaaaaagaccg
aagtacagaccggaggcttctccaaggaaagtatcctcccgaaaaggaacagcgacaagctgatcgca
cgcaaaaaagattgggaccccaagaaatacggcggattcgattctcctacagtcgcttacagtgtact
ggttgtggccaaagtggagaaagggaagtctaaaaaactcaaaagcgtcaaggaactgctgggcatca
caatcatggagcgatcaagcttcgaaaaaaaccccatcgactttctcgaggcgaaaggatataaagag
gtcaaaaaagacctcatcattaagcttcccaagtactctctctttgagcttgaaaacggccggaaacg
aatgctcgctagtgcgggcgagctgcagaaaggtaacgagctggcactgccctctaaatacgttaatt
tcttgtatctggccagccactatgaaaagctcaaagggtctcccgaagataatgagcagaagcagctg
ttcgtggaacaacacaaacactaccttgatgagatcatcgagcaaataagcgaattctccaaaagagt
gatcctcgccgacgctaacctcgataaggtgctttctgcttacaataagcacagggataagcccatca
gggagcaggcagaaaacattatccacttgtttactctgaccaacttgggcgcgcctgcagccttcaag
tacttcgacaccaccatagacagaaagcggtacacctctacaaaggaggtcctggacgccacactgat
tcatcagtcaattacggggctctatgaaacaagaatcgacctctctcagctcggtggagacagcaggg
ctgaccccaagaagaagaggaaggtggctagcgatgctaagtcactgactgcctggtcccggacactg
gtgaccttcaaggatgtgtttgtggacttcaccagggaggagtggaagctgctggacactgctcagca
gatcctgtacagaaatgtgatgctggagaactataagaacctggtttccttgggttatcagcttacta
agccagatgtgatcctccggttggagaagggagaagagccctggctggtggagagagaaattcaccaa
gagacccatcctgattcagagactgcatttgaaatcaaatcatcagttccgaaaaagaaacgcaaagt
ttga
Polypeptide sequence of Staphylococcus aureus dCas9-KRAB protein
SEQ ID NO: 49
MAPKKKRKVGIHGVPAAKRNYILGLAIGITSVGYGIIDYETRDVIDAGVRLFKEANVENNEGRRSKRG
ARRLKRRRRHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYAYNADLY
NALNDLNNLVITRDENEKLEYYEKFQIIENVEKQKKKPTLKQIAKEILVNEEDIKGYRVTSTGKPEFT
NLKVYHDIKDITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVKRSFIQSIKV
INAIIKKYGLPNDIIIELAREKNSKDAQKMINEMQKRNRQTNERIEEIIRTTGKENAKYLIEKIKLHD
MQEGKCLYSLEAIPLEDLLNNPFNYEVDHIIPRSVSFDNSFNNKVLVKQEEASKKGNRTPFQYLSSSD
SKISYETFKKHILNLAKGKGRISKTKKEYLLEERDINRESVQKDFINRNLVDTRYATRGLMNLLRSYF
RVNNLDVKVKSINGGFTSFLRRKWKFKKERNKGYKHHAEDALIIANADFIFKEWKKLDKAKKVMENQM
FEEKQAESMPEIETEQEYKEIFITPHQIKHIKDEKDYKYSHRVDKKPNRELINDTLYSTRKDDKGNTL
IVNNLNGLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKNLDVIKK
ENYYEVNSKCYEEAKKLKKISNQAEFIASFYNNDLIKINGELYRVIGVNNDLLNRIEVNMIDITYREY
LENMNDKRPPRIIKTIASKTQSIKKYSTDILGNLYEVKSKKHPQIIKKGKRPAATKKAGQAKKKKGSD
AKSLTAWSRTLVTFKDVFVDFTREEWKLLDTAQQILYRNVMLENYKNLVSLGYQLTKPDVILRLEKGE
EPWLVEREIHQETHPDSETAFEIKSSVPKKKRKV
Polynucleotide sequence of Staphylococcus aureus dCas9-KRAB protein
SEQ ID NO: 50
atggccccaaagaagaagcggaaggtcggtatccacggagtcccagcagccaagcggaactacatcct
gggcctggccatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaagccagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaagggatccgat
gctaagtcactgactgcctggtcccggacactggtgaccttcaaggatgtgtttgtggacttcaccag
ggaggagtggaagctgctggacactgctcagcagatcctgtacagaaatgtgatgctggagaactata
agaacctggtttccttgggttatcagcttactaagccagatgtgatcctccggttggagaagggagaa
gagccctggctggtggagagagaaattcaccaagagacccatcctgattcagagactgcatttgaaat
caaatcatcagttccgaaaaagaaacgcaaagtt
Polypeptide sequence of Tet1CD
SEQ ID NO: 51
LPTCSCLDRVIQKDKGPYYTHLGAGPSVAAVREIMENRYGQKGNAIRIEIVVYTGKEGKSSHGCPIAK
WVLRRSSDEEKVLCLVRQRTGHHCPTAVMVVLIMVWDGIPLPMADRLYTELTENLKSYNGHPTDRRCT
LNENRTCTCQGIDPETCGASESFGCSWSMYENGCKFGRSPSPRRFRIDPSSPLHEKNLEDNLQSLATR
LAPIYKQYAPVAYQNQVEYENVARECRLGSKEGRPFSGVTACLDFCAHPHRDIHNMNNGSTVVCTLTR
EDNRSLGVIPQDEQLHVLPLYKLSDTDEFGSKEGMEAKIKSGAIEVLAPRRKKRTCFTQPVPRSGKKR
AAMMTEVLAHKIRAVEKKPIPRIKRKNNSTTTNNSKPSSLPTLGSNTETVQPEVKSETEPHFILKSSD
NTKTYSLMPSAPHPVKEASPGFSWSPKTASATPAPLKNDATASCGFSERSSTPHCTMPSGRLSGANAA
AADGPGISQLGEVAPLPTLSAPVMEPLINSEPSTGVTEPLTPHQPNHQPSELTSPQDLASSPMEEDEQ
HSEADEPPSDEPLSDDPLSPAEEKLPHIDEYWSDSEHIFLDANIGGVAIAPAHGSVLIECARRELHAT
TPVEHPNRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWV
Polynucleotide sequence of Tet1CD
SEQ ID NO: 52
CTGCCCACCTGCAGCTGTCTTGATCGAGTTATACAAAAAGACAAAGGCCCATATTATACACACCTTGG
GGCAGGACCAAGTGTTGCTGCTGTCAGGGAAATCATGGAGAATAGGTATGGTCAAAAAGGAAACGCAA
TAAGGATAGAAATAGTAGTGTACACCGGTAAAGAAGGGAAAAGCTCTCATGGGTGTCCAATTGCTAAG
TGGGTTTTAAGAAGAAGCAGTGATGAAGAAAAAGTTCTTTGTTTGGTCCGGCAGCGTACAGGCCACCA
CTGTCCAACTGCTGTGATGGTGGTGCTCATCATGGTGTGGGATGGCATCCCTCTTCCAATGGCCGACC
GGCTATACACAGAGCTCACAGAGAATCTAAAGTCATACAATGGGCACCCTACCGACAGAAGATGCACC
CTCAATGAAAATCGTACCTGTACATGTCAAGGAATTGATCCAGAGACTTGTGGAGCTTCATTCTCTTT
TGGCTGTTCATGGAGTATGTACTTTAATGGCTGTAAGTTTGGTAGAAGCCCAAGCCCCAGAAGATTTA
GAATTGATCCAAGCTCTCCCTTACATGAAAAAAACCTTGAAGATAACTTACAGAGTTTGGCTACACGA
TTAGCTCCAATTTATAAGCAGTATGCTCCAGTAGCTTACCAAAATCAGGTGGAATATGAAAATGTTGC
CCGAGAATGTCGGCTTGGCAGCAAGGAAGGTCGACCCTTCTCTGGGGTCACTGCTTGCCTGGACTTCT
GTGCTCATCCCCACAGGGACATTCACAACATGAATAATGGAAGCACTGTGGTTTGTACCTTAACTCGA
GAAGATAACCGCTCTTTGGGTGTTATTCCTCAAGATGAGCAGCTCCATGTGCTACCTCTTTATAAGCT
TTCAGACACAGATGAGTTTGGCTCCAAGGAAGGAATGGAAGCCAAGATCAAATCTGGGGCCATCGAGG
TCCTGGCACCCCGCCGCAAAAAAAGAACGTGTTTCACTCAGCCTGTTCCCCGTTCTGGAAAGAAGAGG
GCTGCGATGATGACAGAGGTTCTTGCACATAAGATAAGGGCAGTGGAAAAGAAACCTATTCCCCGAAT
CAAGCGGAAGAATAACTCAACAACAACAAACAACAGTAAGCCTTCGTCACTGCCAACCTTAGGGAGTA
ACACTGAGACCGTGCAACCTGAAGTAAAAAGTGAAACCGAACCCCATTTTATCTTAAAAAGTTCAGAC
AACACTAAAACTTATTCGCTGATGCCATCCGCTCCTCACCCAGTGAAAGAGGCATCTCCAGGCTTCTC
CTGGTCCCCGAAGACTGCTTCAGCCACACCAGCTCCACTGAAGAATGACGCAACAGCCTCATGCGGGT
TTTCAGAAAGAAGCAGCACTCCCCACTGTACGATGCCTTCGGGAAGACTCAGTGGTGCCAATGCTGCA
GCTGCTGATGGCCCTGGCATTTCACAGCTTGGCGAAGTGGCTCCTCTCCCCACCCTGTCTGCTCCTGT
GATGGAGCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGATGAGCAG
CATTCTGAAGCAGATGAGCCTCCATCAGACGAACCCCTATCTGATGACCCCCTGTCACCTGCTGAGGA
GAAATTGCCCCACATTGATGAGTATTGGTCAGACAGTGAGCACATCTTTTTGGATGCAAATATTGGTG
GGGTGGCCATCGCACCTGCTCACGGCTCGGTTTTGATTGAGTGTGCCCGGCGAGAGCTGCACGCTACC
ACTCCTGTTGAGCACCCCAACCGTAATCATCCAACCCGCCTCTCCCTTGTCTTTTACCAGCACAAAAA
CCTAAATAAGCCCCAACATGGTTTTGAACTAAACAAGATTAAGTTTGAGGCTAAAGAAGCTAAGAATA
AGAAAATGAAGGCCTCAGAGCAAAAAGACCAGGCAGCTAATGAAGGTCCAGAACAGTCCTCTGAAGTA
AATGAATTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
Protein sequence for VPH
SEQ ID NO: 53
DALDDFDLDMLGSDALDDFDLDMLGSDALDDEDLDMLGSDALDDEDLDMLGSLPSASVEFEGSGGPSG
QISNQALALAPSSAPVLAQTMVPSSAMVPLAQPPAPAPVLTPGPPQSLSAPVPKSTQAGEGTLSEALL
HLQFDADEDLGALLGNSTDPGVFTDLASVDNSEFQQLLNQGVSMSHSTAEPMLMEYPEAITRLVTGSQ
RPPDPAPTPLGTSGLPNGLSGDEDESSIADMDESALLSQISSSGQGGGGSGFSVDTSALLDLESPSVT
VPDMSLPDLDSSLASIQELLSPQEPPRPPEAENSSPDSGKQLVHYTAQPLFLLDPGSVDTGSNDLPVL
FELGEGSYFSEGDGFAEDPTISLLTGSEPPKAKDPTVS
DNA sequence for VPH
SEQ ID NO: 54
Gatgctttagacgattttgacttagatatgcttggttcagacgcgttagacgacttcgacctagacat
gttaggctcagatgcattggacgacttcgatttagatatgttgggctccgatgccctagatgactttg
atctagatatgctagggtcactacccagcgccagcgtcgagttcgaaggcagcggcgggccttcaggg
cagatcagcaaccaggccctggctctggcccctagctccgctccagtgctggcccagactatggtgcc
ctctagtgctatggtgcctctggcccagccacctgctccagcccctgtgctgaccccaggaccacccc
agtcactgagcgccccagtgcccaagtctacacaggccggcgaggggactctgagtgaagctctgctg
cacctgcagttcgacgctgatgaggacctgggagctctgctggggaacagcaccgatcccggagtgtt
cacagatctggcctccgtggacaactctgagtttcagcagctgctgaatcagggcgtgtccatgtctc
atagtacagccgaaccaatgctgatggagtaccccgaagccattacccggctggtgaccggcagccag
cggccccccgaccccgctccaactcccctgggaaccagcggcctgcctaatgggctgtccggagatga
agacttctcaagcatcgctgatatggactttagtgccctgctgtcacagatttcctctagtgggcagg
gaggaggtggaagcggcttcagcgtggacaccagtgccctgctggacctgttcagcccctcggtgacc
gtgcccgacatgagcctgcctgaccttgacagcagcctggccagtatccaagagctcctgtctcccca
ggagccccccaggcctcccgaggcagagaacagcagcccggattcagggaagcagctggtgcactaca
cagcgcagccgctgttcctgctggaccccggctccgtggacaccgggagcaacgacctgccggtgctg
tttgagctgggagagggctcctacttctccgaaggggacggcttcgccgaggaccccaccatctccct
gctgacaggctcggagcctcccaaagccaaggaccccactgtctcc
Protein sequence for VPR
SEQ ID NO: 55
DALDDFDLDMLGSDALDDFDLDMLGSDALDDEDLDMLGSDALDDEDLDMLGSPKKKRKVGSQYLPDTD
DRHRIEEKRKRTYETFKSIMKKSPFSGPTDPRPPPRRIAVPSRSSASVPKPAPQPYPFTSSLSTINYD
EFPTMVFPSGQISQASALAPAPPQVLPQAPAPAPAPAMVSALAQAPAPVPVLAPGPPQAVAPPAPKPT
QAGEGTLSEALLQLQFDDEDLGALLGNSTDPAVFTDLASVDNSEFQQLLNQGIPVAPHTTEPMLMEYP
EAITRLVTGAQRPPDPAPAPLGAPGLPNGLLSGDEDESSIADMDESALLSQISSGSGSGSRDSREGME
LPKPEAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTTTLES
MTEDLNLDSPLTPELNEILDTELNDECLLHAMHISTGLSIFDTSLE
DNA sequence for VPR
SEQ ID NO: 56
gatgctttagacgattttgacttagatatgcttggttcagacgcgttagacgacttcgacctagacat
gttaggctcagatgcattggacgacttcgatttagatatgttgggctccgatgccctagatgactttg
atctagatatgctaggtagtcccaaaaagaagaggaaagtgggatcccagtatctgcccgacacagat
gatagacaccgaatcgaagagaaacgcaagcgaacgtatgaaaccttcaaatcgatcatgaagaaatc
gcccttctcgggtccgaccgatcccaggcccccaccgagaaggattgcggtcccgtcccgctcgtcgg
ccagcgtgccgaagcctgcgccgcagccctaccccttcacgtcgagcctgagcacaatcaattatgac
gagttcccgacgatggtgttcccctcgggacaaatctcacaagcctcggcgctcgcaccagcgcctcc
ccaagtccttccgcaagcgcctgccccagcgcctgcaccggcaatggtgtccgccctcgcacaggccc
ctgcgcccgtccccgtgctcgcgcctggaccgccccaggcggtcgctccaccggctccgaagccgacg
caggccggagagggaacactctccgaagcacttcttcaactccagtttgatgacgaggatcttggagc
actccttggaaactcgacagaccctgcggtgtttaccgacctcgcgtcagtagataactccgaatttc
agcagcttttgaaccagggtatcccggtcgcgccacatacaacggagcccatgttgatggaatacccc
gaagcaatcacgagacttgtgacgggagcgcagcggcctcccgatcccgcacccgcacctttgggggc
acctggcctccctaacggacttttgagcggcgacgaggatttctcctccatcgccgatatggatttct
cagccttgctgtcacagatttccagcggctctggcagcggcagccgggattccagggaagggatgttt
ttgccgaagcctgaggccggctccgctattagtgacgtgtttgagggccgcgaggtgtgccagccaaa
acgaatccggccatttcatcctccaggaagtccatgggccaaccgcccactccccgccagcctcgcac
caacaccaaccggtccagtacatgagccagtcgggtcactgaccccggcaccagtccctcagccactg
gatccagcgcccgcagtgactcccgaggccagtcacctgttggaggatcccgatgaagagacgagcca
ggctgtcaaagcccttcgggagatggccgatactgtgattccccagaaggaagaggctgcaatctgtg
gccaaatggacctttcccatccgcccccaaggggccatctggatgagctgacaaccacacttgagtcc
atgaccgaggatctgaacctggactcacccctgaccccggaattgaacgagattctggataccttcct
gaacgacgagtgcctcttgcatgccatgcatatcagcacaggactgtccatcttcgacacatctctgt
tt
Amino acid sequence of S. uberis Cas9 nuclease
SEQ ID NO: 57
MTNGMILGLDIGVASVGVGIIEADSGKVIHASSRIFPSANADNNVERRKFRGSRRLLRRKKH
RVKRLQDLFDKYDIVTNFDNLNLNPYELRVKGLNEPLSNEELFASLRNITKHRGISYLDDAE
DDSSGNGTEYAKAIELNQQLLKEKTPGQIQYDRLNQYGQLRGNFDIVDENGEIHHVINVEST
SSYRKEAEQILKKQSETNTSISTDFINDFIQLLTSKRKYYHGPGNPKSRTDYGRYRTDGTDL
DNIFDVLIGKCSFYPEEYRASKTSYTAQEFNFLNDLNNLTLPTETGKLSEQQKIDLVNWAKE
TKILGPKKLLQEIAKRNNCKYEDIRGYRLDKKDNPDMHVFDVYRKMNEDLETISVKDLSVDS
LNQLARILTLNTEREGIEEAIKNLMPNQFTEKQMLELIAFRKSNSSIFGKGWHSLSIKLMKE
LIPELYHTSDEQMTILNRFGKFKLTKLDSKRTNYIDENFVTDEIYNPVVAKSVRQAIKIINA
SIKKWGDFDKIVIEMPRDKNEEEERKRIADGQKVNAKEKEQAEKHAAKLFNGKEELPSEVFH
GYKELALRIRLWYQQDQKCLYSGKEITISDLIYNRELFEIDHILPLSLSFDDSLSNKVLVYR
WANQEKGQRTPFQALDSMKSAWSYREFKNAILHNSKISRRKRDYFLTEQDISKIEVKQKFIE
RNLVDTRYASRTVLNVLQQSLKNLEKETKVSVVRGQFTSQLRRKWHIDKTRDTYHHHAVDAL
IIAASAKLRYWKKQGDILFENYLINRHVDRVTGEIQSDDSYKEEVFTPPYDGFVQTISNPGF
EDEILFSYQVDSKVNRKISDATIYATRSAKLEKDKKEQTYVLGKIKDIYSQTGFENFLKIYN
KDKSKFLIYQKDPETWEKIIEPILKNYREFDNKGKDIVNPFEKYRNDNGPICKYSRKGNGPE
IKQFKYYDTVYKITSGLDISPRESRNKVILQSLNPWRTDFYFNPKTMKYELMGIRYVDLEFE
KGTGDYLISDNLYKEIKKNEGISELSVFKFTLYKNDLLLIKDTENNEEQIFRFWSRNDLSSK
NRVELKPYDRSRFSGNEILITKMGKAPKQCIKTLTYQNISIYKIKTDILGFKYYLKNEGNKP
LLHFKK
DNA sequence of S. uberis Cas9 (nuclease-active;
optimized for expression in mammalian cells)
SEQ ID NO: 58
ATGACTAATGGGATGATTCTGGGCTTAGATATCGGAGTCGCGTCTGTAGGGGTAGGAATTAT
CGAGGCCGATAGTGGCAAGGTAATTCACGCAAGCTCACGGATCTTCCCTAGTGCTAATGCCG
ACAATAATGTGGAGCGCCGCAAGTTCCGGGGATCTAGGCGCCTTCTTAGGAGGAAAAAGCAC
AGGGTTAAGCGCTTACAGGATCTGTTTGACAAGTACGATATCGTGACTAACTTCGATAACCT
CAACCTTAACCCCTACGAGCTGCGAGTTAAGGGCTTAAACGAGCCATTGAGCAACGAAGAGC
TCTTCGCATCACTCCGGAACATCACAAAGCACAGGGGCATTTCCTATCTCGATGATGCTGAG
GATGACTCTTCTGGAAACGGGACAGAATACGCCAAAGCGATAGAGCTGAATCAGCAGCTTCT
GAAAGAAAAGACCCCCGGTCAGATCCAGTACGACAGACTCAATCAGTATGGGCAACTGAGAG
GCAATTTCGATATCGTGGATGAAAACGGCGAGATTCACCACGTGATAAACGTTTTTTCAACA
TCAAGTTACAGAAAGGAAGCCGAGCAGATTCTCAAGAAGCAGTCTGAAACGAATACTAGTAT
CAGCACCGACTTTATAAATGATTTCATCCAATTGCTGACCTCTAAGAGGAAATATTACCATG
GTCCTGGTAATCCAAAGAGCCGCACAGATTACGGGCGCTACCGGACGGATGGGACGGATCTC
GATAACATCTTCGATGTTCTGATAGGTAAATGCAGCTTTTACCCAGAGGAGTACCGAGCCAG
CAAGACGAGCTACACTGCCCAAGAGTTCAACTTTCTTAATGACTTGAATAACCTGACCTTAC
CAACCGAGACAGGCAAGTTGAGCGAGCAGCAGAAGATCGACCTGGTGAATTGGGCTAAGGAG
ACAAAGATCCTCGGACCGAAAAAGCTGCTTCAGGAAATTGCCAAGAGGAACAACTGCAAGTA
CGAGGACATTCGCGGCTATCGGCTTGATAAGAAAGATAACCCCGATATGCATGTATTTGATG
TGTATCGGAAGATGAATTTTGACCTGGAGACTATTTCCGTTAAGGATCTGTCAGTCGACTCT
CTGAATCAGCTCGCGCGAATTCTGACACTGAACACCGAGAGGGAGGGGATCGAAGAGGCCAT
CAAAAATCTGATGCCAAACCAGTTCACCGAGAAGCAAATGCTTGAACTCATCGCCTTCCGCA
AGAGTAATTCCTCTATCTTTGGGAAGGGGTGGCACAGTCTGTCAATTAAACTGATGAAAGAG
CTGATACCCGAGCTCTACCACACCAGTGACGAACAAATGACCATACTCAATCGATTTGGTAA
GTTCAAGCTCACGAAGCTCGACTCAAAAAGGACCAATTACATCGATGAAAACTTTGTCACTG
ATGAAATCTATAACCCTGTAGTGGCCAAGAGTGTGAGGCAGGCAATAAAGATCATCAACGCT
TCCATTAAAAAGTGGGGGGACTTTGATAAGATCGTGATTGAGATGCCACGCGACAAGAATGA
GGAGGAGGAAAGGAAACGAATCGCCGATGGCCAGAAGGTGAATGCTAAGGAAAAAGAGCAGG
CCGAGAAGCACGCCGCAAAGCTCTTTAATGGCAAGGAAGAGCTCCCTTCTGAAGTTTTCCAT
GGATATAAGGAGCTGGCTTTGCGAATTAGACTCTGGTATCAGCAAGACCAGAAGTGCCTCTA
TTCTGGCAAGGAGATAACAATTTCAGACCTGATCTACAACAGGGAGCTCTTTGAGATTGACC
ATATCCTTCCGCTGTCTCTTTCTTTTGACGACAGTCTGTCTAACAAGGTCCTGGTTTACAGA
TGGGCAAATCAGGAGAAGGGCCAGAGGACCCCTTTCCAAGCCCTTGATTCCATGAAATCAGC
GTGGTCCTATCGGGAGTTCAAGAATGCAATCCTGCACAATTCTAAAATCAGCCGGAGAAAGC
GTGACTATTTTCTGACAGAACAAGACATTAGTAAGATTGAGGTGAAACAAAAGTTTATTGAG
AGGAACTTGGTGGACACACGGTACGCCAGTAGAACAGTTCTCAACGTGCTGCAGCAGTCCCT
GAAGAATCTGGAGAAGGAGACTAAGGTGTCCGTTGTCCGAGGACAGTTCACGTCCCAGCTGC
GCCGGAAATGGCACATAGATAAGACCAGGGATACTTACCATCACCATGCGGTGGACGCACTG
ATTATCGCGGCCTCCGCTAAGTTGAGATATTGGAAGAAACAGGGCGACATCTTGTTCGAGAA
CTATCTCATCAATCGCCACGTAGATAGAGTAACCGGGGAGATACAATCTGACGATAGCTATA
AGGAGGAGGTGTTCACACCTCCCTACGACGGATTTGTCCAGACTATTAGCAACCCAGGGTTT
GAGGACGAGATCCTTTTCTCCTATCAGGTAGACAGTAAAGTCAACAGAAAGATCTCAGACGC
CACGATATACGCTACGAGGTCTGCGAAGCTCGAGAAGGACAAGAAGGAACAGACGTATGTCT
TGGGTAAGATAAAAGATATCTATTCACAAACTGGTTTTGAGAACTTCCTGAAGATCTATAAT
AAGGACAAGAGTAAGTTCCTGATCTACCAGAAGGACCCTGAGACTTGGGAAAAGATCATTGA
ACCAATTCTCAAAAATTATCGGGAATTCGATAATAAAGGCAAGGATATCGTGAATCCATTTG
AGAAATACAGGAATGATAACGGGCCTATCTGCAAGTACAGTCGGAAAGGCAACGGCCCTGAG
ATCAAACAATTTAAATACTACGACACCGTTTACAAAATTACAAGCGGTCTCGACATCAGCCC
CCGCGAATCAAGAAATAAGGTAATTCTTCAAAGCCTGAATCCGTGGAGAACCGACTTCTACT
TTAACCCTAAGACTATGAAGTACGAACTTATGGGTATCAGATATGTCGACCTGGAGTTCGAG
AAAGGAACAGGGGACTACCTGATTTCTGACAATCTCTATAAAGAGATTAAAAAGAACGAGGG
GATCTCTGAGCTGAGTGTATTCAAGTTCACACTCTACAAGAACGATCTCCTGCTGATCAAGG
ACACTGAGAACAACGAAGAGCAAATTTTTAGGTTTTGGTCTCGGAATGACCTGTCCTCCAAA
AACCGGGTGGAACTGAAGCCCTACGATAGGTCCCGCTTTTCCGGCAATGAGATCCTTATCAC
CAAAATGGGCAAGGCACCTAAGCAATGCATTAAGACTTTAACATACCAAAACATCTCCATTT
ATAAAATCAAAACAGACATCCTGGGATTCAAATACTATCTGAAAAACGAAGGAAATAAGCCA
TTACTGCACTTTAAGAAG
Amino acid sequence of S. uberis dCas9
(with D10A and H600A underlined)
SEQ ID NO: 59
MTNGMILGLAIGVASVGVGIIEADSGKVIHASSRIFPSANADNNVERRKERGSRRLLRRKKH
RVKRLQDLFDKYDIVINFDNLNLNPYELRVKGLNEPLSNEELFASLRNITKHRGISYLDDAE
DDSSGNGTEYAKAIELNQQLLKEKTPGQIQYDRLNQYGQLRGNFDIVDENGEIHHVINVEST
SSYRKEAEQILKKQSETNTSISTDFINDFIQLLTSKRKYYHGPGNPKSRTDYGRYRTDGTDL
DNIFDVLIGKCSFYPEEYRASKTSYTAQEFNFLNDLNNLTLPTETGKLSEQQKIDLVNWAKE
TKILGPKKLLQEIAKRNNCKYEDIRGYRLDKKDNPDMHVEDVYRKMNEDLETISVKDLSVDS
LNQLARILTLNTEREGIEEAIKNLMPNQFTEKQMLELIAFRKSNSSIFGKGWHSLSIKLMKE
LIPELYHTSDEQMTILNRFGKFKLTKLDSKRINYIDENFVTDEIYNPVVAKSVRQAIKIINA
SIKKWGDFDKIVIEMPRDKNEEEERKRIADGQKVNAKEKEQAEKHAAKLENGKEELPSEVFH
GYKELALRIRLWYQQDQKCLYSGKEITISDLIYNRELFEIDAILPLSLSFDDSLSNKVLVYR
WANQEKGQRTPFQALDSMKSAWSYREFKNAILHNSKISRRKRDYFLTEQDISKIEVKQKFIE
RNLVDTRYASRTVLNVLQQSLKNLEKETKVSVVRGQFTSQLRRKWHIDKTRDTYHHHAVDAL
IIAASAKLRYWKKQGDILFENYLINRHVDRVTGEIQSDDSYKEEVFTPPYDGFVQTISNPGF
EDEILFSYQVDSKVNRKISDATIYATRSAKLEKDKKEQTYVLGKIKDIYSQTGFENFLKIYN
KDKSKFLIYQKDPETWEKIIEPILKNYREFDNKGKDIVNPFEKYRNDNGPICKYSRKGNGPE
IKQFKYYDTVYKITSGLDISPRESRNKVILQSLNPWRTDFYFNPKTMKYELMGIRYVDLEFE
KGTGDYLISDNLYKEIKKNEGISELSVFKFTLYKNDLLLIKDTENNEEQIFREWSRNDLSSK
NRVELKPYDRSRFSGNEILITKMGKAPKQCIKTLTYQNISIYKIKTDILGFKYYLKNEGNKP
LLHFKK
DNA sequence of S. uberis dCas9 (human codon-optimized)
SEQ ID NO: 60
ATGACTAATGGGATGATTCTGGGCTTAGCAATCGGAGTCGCGTCTGTAGGGGTAGGAATTAT
CGAGGCCGATAGTGGCAAGGTAATTCACGCAAGCTCACGGATCTTCCCTAGTGCTAATGCCG
ACAATAATGTGGAGCGCCGCAAGTTCCGGGGATCTAGGCGCCTTCTTAGGAGGAAAAAGCAC
AGGGTTAAGCGCTTACAGGATCTGTTTGACAAGTACGATATCGTGACTAACTTCGATAACCT
CAACCTTAACCCCTACGAGCTGCGAGTTAAGGGCTTAAACGAGCCATTGAGCAACGAAGAGC
TCTTCGCATCACTCCGGAACATCACAAAGCACAGGGGCATTTCCTATCTCGATGATGCTGAG
GATGACTCTTCTGGAAACGGGACAGAATACGCCAAAGCGATAGAGCTGAATCAGCAGCTTCT
GAAAGAAAAGACCCCCGGTCAGATCCAGTACGACAGACTCAATCAGTATGGGCAACTGAGAG
GCAATTTCGATATCGTGGATGAAAACGGCGAGATTCACCACGTGATAAACGTTTTTTCAACA
TCAAGTTACAGAAAGGAAGCCGAGCAGATTCTCAAGAAGCAGTCTGAAACGAATACTAGTAT
CAGCACCGACTTTATAAATGATTTCATCCAATTGCTGACCTCTAAGAGGAAATATTACCATG
GTCCTGGTAATCCAAAGAGCCGCACAGATTACGGGCGCTACCGGACGGATGGGACGGATCTC
GATAACATCTTCGATGTTCTGATAGGTAAATGCAGCTTTTACCCAGAGGAGTACCGAGCCAG
CAAGACGAGCTACACTGCCCAAGAGTTCAACTTTCTTAATGACTTGAATAACCTGACCTTAC
CAACCGAGACAGGCAAGTTGAGCGAGCAGCAGAAGATCGACCTGGTGAATTGGGCTAAGGAG
ACAAAGATCCTCGGACCGAAAAAGCTGCTTCAGGAAATTGCCAAGAGGAACAACTGCAAGTA
CGAGGACATTCGCGGCTATCGGCTTGATAAGAAAGATAACCCCGATATGCATGTATTTGATG
TGTATCGGAAGATGAATTTTGACCTGGAGACTATTTCCGTTAAGGATCTGTCAGTCGACTCT
CTGAATCAGCTCGCGCGAATTCTGACACTGAACACCGAGAGGGAGGGGATCGAAGAGGCCAT
CAAAAATCTGATGCCAAACCAGTTCACCGAGAAGCAAATGCTTGAACTCATCGCCTTCCGCA
AGAGTAATTCCTCTATCTTTGGGAAGGGGTGGCACAGTCTGTCAATTAAACTGATGAAAGAG
CTGATACCCGAGCTCTACCACACCAGTGACGAACAAATGACCATACTCAATCGATTTGGTAA
GTTCAAGCTCACGAAGCTCGACTCAAAAAGGACCAATTACATCGATGAAAACTTTGTCACTG
ATGAAATCTATAACCCTGTAGTGGCCAAGAGTGTGAGGCAGGCAATAAAGATCATCAACGCT
TCCATTAAAAAGTGGGGGGACTTTGATAAGATCGTGATTGAGATGCCACGCGACAAGAATGA
GGAGGAGGAAAGGAAACGAATCGCCGATGGCCAGAAGGTGAATGCTAAGGAAAAAGAGCAGG
CCGAGAAGCACGCCGCAAAGCTCTTTAATGGCAAGGAAGAGCTCCCTTCTGAAGTTTTCCAT
GGATATAAGGAGCTGGCTTTGCGAATTAGACTCTGGTATCAGCAAGACCAGAAGTGCCTCTA
TTCTGGCAAGGAGATAACAATTTCAGACCTGATCTACAACAGGGAGCTCTTTGAGATTGACG
CCATCCTTCCGCTGTCTCTTTCTTTTGACGACAGTCTGTCTAACAAGGTCCTGGTTTACAGA
TGGGCAAATCAGGAGAAGGGCCAGAGGACCCCTTTCCAAGCCCTTGATTCCATGAAATCAGC
GTGGTCCTATCGGGAGTTCAAGAATGCAATCCTGCACAATTCTAAAATCAGCCGGAGAAAGC
GTGACTATTTTCTGACAGAACAAGACATTAGTAAGATTGAGGTGAAACAAAAGTTTATTGAG
AGGAACTTGGTGGACACACGGTACGCCAGTAGAACAGTTCTCAACGTGCTGCAGCAGTCCCT
GAAGAATCTGGAGAAGGAGACTAAGGTGTCCGTTGTCCGAGGACAGTTCACGTCCCAGCTGC
GCCGGAAATGGCACATAGATAAGACCAGGGATACTTACCATCACCATGCGGTGGACGCACTG
ATTATCGCGGCCTCCGCTAAGTTGAGATATTGGAAGAAACAGGGCGACATCTTGTTCGAGAA
CTATCTCATCAATCGCCACGTAGATAGAGTAACCGGGGAGATACAATCTGACGATAGCTATA
AGGAGGAGGTGTTCACACCTCCCTACGACGGATTTGTCCAGACTATTAGCAACCCAGGGTTT
GAGGACGAGATCCTTTTCTCCTATCAGGTAGACAGTAAAGTCAACAGAAAGATCTCAGACGC
CACGATATACGCTACGAGGTCTGCGAAGCTCGAGAAGGACAAGAAGGAACAGACGTATGTCT
TGGGTAAGATAAAAGATATCTATTCACAAACTGGTTTTGAGAACTTCCTGAAGATCTATAAT
AAGGACAAGAGTAAGTTCCTGATCTACCAGAAGGACCCTGAGACTTGGGAAAAGATCATTGA
ACCAATTCTCAAAAATTATCGGGAATTCGATAATAAAGGCAAGGATATCGTGAATCCATTTG
AGAAATACAGGAATGATAACGGGCCTATCTGCAAGTACAGTCGGAAAGGCAACGGCCCTGAG
ATCAAACAATTTAAATACTACGACACCGTTTACAAAATTACAAGCGGTCTCGACATCAGCCC
CCGCGAATCAAGAAATAAGGTAATTCTTCAAAGCCTGAATCCGTGGAGAACCGACTTCTACT
TTAACCCTAAGACTATGAAGTACGAACTTATGGGTATCAGATATGTCGACCTGGAGTTCGAG
AAAGGAACAGGGGACTACCTGATTTCTGACAATCTCTATAAAGAGATTAAAAAGAACGAGGG
GATCTCTGAGCTGAGTGTATTCAAGTTCACACTCTACAAGAACGATCTCCTGCTGATCAAGG
ACACTGAGAACAACGAAGAGCAAATTTTTAGGTTTTGGTCTCGGAATGACCTGTCCTCCAAA
AACCGGGTGGAACTGAAGCCCTACGATAGGTCCCGCTTTTCCGGCAATGAGATCCTTATCAC
CAAAATGGGCAAGGCACCTAAGCAATGCATTAAGACTTTAACATACCAAAACATCTCCATTT
ATAAAATCAAAACAGACATCCTGGGATTCAAATACTATCTGAAAAACGAAGGAAATAAGCCA
TTACTGCACTTTAAGAAG
Amino acid sequence of S. uberis dCas9-KRAB
SEQ ID NO: 61
MTNGMILGLAIGVASVGVGIIEADSGKVIHASSRIFPSANADNNVERRKFRGSRRLLRRKKH
RVKRLQDLFDKYDIVTNFDNLNLNPYELRVKGLNEPLSNEELFASLRNITKHRGISYLDDAE
DDSSGNGTEYAKAIELNQQLLKEKTPGQIQYDRLNQYGQLRGNFDIVDENGEIHHVINVEST
SSYRKEAEQILKKQSETNTSISTDFINDFIQLLTSKRKYYHGPGNPKSRTDYGRYRTDGTDL
DNIFDVLIGKCSFYPEEYRASKTSYTAQEFNFLNDLNNLTLPTETGKLSEQQKIDLVNWAKE
TKILGPKKLLQEIAKRNNCKYEDIRGYRLDKKDNPDMHVFDVYRKMNEDLETISVKDLSVDS
LNQLARILTLNTEREGIEEAIKNLMPNQFTEKQMLELIAFRKSNSSIFGKGWHSLSIKLMKE
LIPELYHTSDEQMTILNRFGKFKLTKLDSKRINYIDENFVTDEIYNPVVAKSVRQAIKIINA
SIKKWGDFDKIVIEMPRDKNEEEERKRIADGQKVNAKEKEQAEKHAAKLENGKEELPSEVFH
GYKELALRIRLWYQQDQKCLYSGKEITISDLIYNRELFEIDAILPLSLSFDDSLSNKVLVYR
WANQEKGQRTPFQALDSMKSAWSYREFKNAILHNSKISRRKRDYFLTEQDISKIEVKQKFIE
RNLVDTRYASRTVLNVLQQSLKNLEKETKVSVVRGQFTSQLRRKWHIDKTRDTYHHHAVDAL
IIAASAKLRYWKKQGDILFENYLINRHVDRVTGEIQSDDSYKEEVFTPPYDGFVQTISNPGF
EDEILFSYQVDSKVNRKISDATIYATRSAKLEKDKKEQTYVLGKIKDIYSQTGFENELKIYN
KDKSKFLIYQKDPETWEKIIEPILKNYREFDNKGKDIVNPFEKYRNDNGPICKYSRKGNGPE
IKQFKYYDTVYKITSGLDISPRESRNKVILQSLNPWRTDFYFNPKTMKYELMGIRYVDLEFE
KGTGDYLISDNLYKEIKKNEGISELSVFKFTLYKNDLLLIKDTENNEEQIFRFWSRNDLSSK
NRVELKPYDRSRFSGNEILITKMGKAPKQCIKTLTYQNISIYKIKTDILGFKYYLKNEGNKP
LLHFKKTGPKKKRKVASMDAKSLTAWSRTLVTFKDVFVDFTREEWKLLDTAQQIVYRNVMLE
NYKNLVSLGYQLTKPDVILRLEKGEEP
DNA sequence of S. uberis dCas9-KRAB (human codon optimized)
SEQ ID NO: 62
ATGACTAATGGGATGATTCTGGGCTTAGCAATCGGAGTCGCGTCTGTAGGGGTAGGAATTAT
CGAGGCCGATAGTGGCAAGGTAATTCACGCAAGCTCACGGATCTTCCCTAGTGCTAATGCCG
ACAATAATGTGGAGCGCCGCAAGTTCCGGGGATCTAGGCGCCTTCTTAGGAGGAAAAAGCAC
AGGGTTAAGCGCTTACAGGATCTGTTTGACAAGTACGATATCGTGACTAACTTCGATAACCT
CAACCTTAACCCCTACGAGCTGCGAGTTAAGGGCTTAAACGAGCCATTGAGCAACGAAGAGC
TCTTCGCATCACTCCGGAACATCACAAAGCACAGGGGCATTTCCTATCTCGATGATGCTGAG
GATGACTCTTCTGGAAACGGGACAGAATACGCCAAAGCGATAGAGCTGAATCAGCAGCTTCT
GAAAGAAAAGACCCCCGGTCAGATCCAGTACGACAGACTCAATCAGTATGGGCAACTGAGAG
GCAATTTCGATATCGTGGATGAAAACGGCGAGATTCACCACGTGATAAACGTTTTTTCAACA
TCAAGTTACAGAAAGGAAGCCGAGCAGATTCTCAAGAAGCAGTCTGAAACGAATACTAGTAT
CAGCACCGACTTTATAAATGATTTCATCCAATTGCTGACCTCTAAGAGGAAATATTACCATG
GTCCTGGTAATCCAAAGAGCCGCACAGATTACGGGCGCTACCGGACGGATGGGACGGATCTC
GATAACATCTTCGATGTTCTGATAGGTAAATGCAGCTTTTACCCAGAGGAGTACCGAGCCAG
CAAGACGAGCTACACTGCCCAAGAGTTCAACTTTCTTAATGACTTGAATAACCTGACCTTAC
CAACCGAGACAGGCAAGTTGAGCGAGCAGCAGAAGATCGACCTGGTGAATTGGGCTAAGGAG
ACAAAGATCCTCGGACCGAAAAAGCTGCTTCAGGAAATTGCCAAGAGGAACAACTGCAAGTA
CGAGGACATTCGCGGCTATCGGCTTGATAAGAAAGATAACCCCGATATGCATGTATTTGATG
TGTATCGGAAGATGAATTTTGACCTGGAGACTATTTCCGTTAAGGATCTGTCAGTCGACTCT
CTGAATCAGCTCGCGCGAATTCTGACACTGAACACCGAGAGGGAGGGGATCGAAGAGGCCAT
CAAAAATCTGATGCCAAACCAGTTCACCGAGAAGCAAATGCTTGAACTCATCGCCTTCCGCA
AGAGTAATTCCTCTATCTTTGGGAAGGGGTGGCACAGTCTGTCAATTAAACTGATGAAAGAG
CTGATACCCGAGCTCTACCACACCAGTGACGAACAAATGACCATACTCAATCGATTTGGTAA
GTTCAAGCTCACGAAGCTCGACTCAAAAAGGACCAATTACATCGATGAAAACTTTGTCACTG
ATGAAATCTATAACCCTGTAGTGGCCAAGAGTGTGAGGCAGGCAATAAAGATCATCAACGCT
TCCATTAAAAAGTGGGGGGACTTTGATAAGATCGTGATTGAGATGCCACGCGACAAGAATGA
GGAGGAGGAAAGGAAACGAATCGCCGATGGCCAGAAGGTGAATGCTAAGGAAAAAGAGCAGG
CCGAGAAGCACGCCGCAAAGCTCTTTAATGGCAAGGAAGAGCTCCCTTCTGAAGTTTTCCAT
GGATATAAGGAGCTGGCTTTGCGAATTAGACTCTGGTATCAGCAAGACCAGAAGTGCCTCTA
TTCTGGCAAGGAGATAACAATTTCAGACCTGATCTACAACAGGGAGCTCTTTGAGATTGACG
CCATCCTTCCGCTGTCTCTTTCTTTTGACGACAGTCTGTCTAACAAGGTCCTGGTTTACAGA
TGGGCAAATCAGGAGAAGGGCCAGAGGACCCCTTTCCAAGCCCTTGATTCCATGAAATCAGC
GTGGTCCTATCGGGAGTTCAAGAATGCAATCCTGCACAATTCTAAAATCAGCCGGAGAAAGC
GTGACTATTTTCTGACAGAACAAGACATTAGTAAGATTGAGGTGAAACAAAAGTTTATTGAG
AGGAACTTGGTGGACACACGGTACGCCAGTAGAACAGTTCTCAACGTGCTGCAGCAGTCCCT
GAAGAATCTGGAGAAGGAGACTAAGGTGTCCGTTGTCCGAGGACAGTTCACGTCCCAGCTGC
GCCGGAAATGGCACATAGATAAGACCAGGGATACTTACCATCACCATGCGGTGGACGCACTG
ATTATCGCGGCCTCCGCTAAGTTGAGATATTGGAAGAAACAGGGCGACATCTTGTTCGAGAA
CTATCTCATCAATCGCCACGTAGATAGAGTAACCGGGGAGATACAATCTGACGATAGCTATA
AGGAGGAGGTGTTCACACCTCCCTACGACGGATTTGTCCAGACTATTAGCAACCCAGGGTTT
GAGGACGAGATCCTTTTCTCCTATCAGGTAGACAGTAAAGTCAACAGAAAGATCTCAGACGC
CACGATATACGCTACGAGGTCTGCGAAGCTCGAGAAGGACAAGAAGGAACAGACGTATGTCT
TGGGTAAGATAAAAGATATCTATTCACAAACTGGTTTTGAGAACTTCCTGAAGATCTATAAT
AAGGACAAGAGTAAGTTCCTGATCTACCAGAAGGACCCTGAGACTTGGGAAAAGATCATTGA
ACCAATTCTCAAAAATTATCGGGAATTCGATAATAAAGGCAAGGATATCGTGAATCCATTTG
AGAAATACAGGAATGATAACGGGCCTATCTGCAAGTACAGTCGGAAAGGCAACGGCCCTGAG
ATCAAACAATTTAAATACTACGACACCGTTTACAAAATTACAAGCGGTCTCGACATCAGCCC
CCGCGAATCAAGAAATAAGGTAATTCTTCAAAGCCTGAATCCGTGGAGAACCGACTTCTACT
TTAACCCTAAGACTATGAAGTACGAACTTATGGGTATCAGATATGTCGACCTGGAGTTCGAG
AAAGGAACAGGGGACTACCTGATTTCTGACAATCTCTATAAAGAGATTAAAAAGAACGAGGG
GATCTCTGAGCTGAGTGTATTCAAGTTCACACTCTACAAGAACGATCTCCTGCTGATCAAGG
ACACTGAGAACAACGAAGAGCAAATTTTTAGGTTTTGGTCTCGGAATGACCTGTCCTCCAAA
AACCGGGTGGAACTGAAGCCCTACGATAGGTCCCGCTTTTCCGGCAATGAGATCCTTATCAC
CAAAATGGGCAAGGCACCTAAGCAATGCATTAAGACTTTAACATACCAAAACATCTCCATTT
ATAAAATCAAAACAGACATCCTGGGATTCAAATACTATCTGAAAAACGAAGGAAATAAGCCA
TTACTGCACTTTAAGAAGACCGGTCCTAAGAAAAAGCGGAAAGTGGctagCatggatgctaa
gtcactaactgcctggtccCGGACACTGGTGACCTTCAAGGATGTATTTGTGGACTTCACCA
GGGAGGAGTGGAAGCTGCTGGACACTGCTCAGCAGATCGTGTACAGAAATGTGATGCTGGAG
AACTATAAGAACCTGGTTTCCTTGGGTTATCAGCTTACTAAGCCAGATGTGATCCTCCGGTT
GGAGAAGGGAGAAGAGCCC
Amino acid sequence of S. uberis Cas9 with His tag
SEQ ID NO: 63
MGHHHHHHGGGSGMDYKDHDGDYKDHDIDYKDDDDKHVNPKKKRKVGRGTGMTNGMILGLDI
GVASVGVGIIEADSGKVIHASSRIFPSANADNNVERRKERGSRRLLRRKKHRVKRLQDLFDK
YDIVTNFDNLNLNPYELRVKGLNEPLSNEELFASLRNITKHRGISYLDDAEDDSSGNGTEYA
KAIELNQQLLKEKTPGQIQYDRLNQYGQLRGNFDIVDENGEIHHVINVESTSSYRKEAEQIL
KKQSETNTSISTDFINDFIQLLTSKRKYYHGPGNPKSRTDYGRYRTDGTDLDNIFDVLIGKC
SFYPEEYRASKTSYTAQEFNFLNDLNNLTLPTETGKLSEQQKIDLVNWAKETKILGPKKLLQ
EIAKRNNCKYEDIRGYRLDKKDNPDMHVFDVYRKMNEDLETISVKDLSVDSLNQLARILTLN
TEREGIEEAIKNLMPNQFTEKQMLELIAFRKSNSSIFGKGWHSLSIKLMKELIPELYHTSDE
QMTILNRFGKFKLTKLDSKRTNYIDENFVTDEIYNPVVAKSVRQAIKIINASIKKWGDFDKI
VIEMPRDKNEEEERKRIADGQKVNAKEKEQAEKHAAKLENGKEELPSEVFHGYKELALRIRL
WYQQDQKCLYSGKEITISDLIYNRELFEIDHILPLSLSFDDSLSNKVLVYRWANQEKGQRTP
FQALDSMKSAWSYREFKNAILHNSKISRRKRDYFLTEQDISKIEVKQKFIERNLVDTRYASR
TVLNVLQQSLKNLEKETKVSVVRGQFTSQLRRKWHIDKTRDTYHHHAVDALIIAASAKLRYW
KKQGDILFENYLINRHVDRVTGEIQSDDSYKEEVFTPPYDGFVQTISNPGFEDEILFSYQVD
SKVNRKISDATIYATRSAKLEKDKKEQTYVLGKIKDIYSQTGFENFLKIYNKDKSKFLIYQK
DPETWEKIIEPILKNYREFDNKGKDIVNPFEKYRNDNGPICKYSRKGNGPEIKQFKYYDTVY
KITSGLDISPRESRNKVILQSLNPWRTDFYFNPKTMKYELMGIRYVDLEFEKGTGDYLISDN
LYKEIKKNEGISELSVFKFTLYKNDLLLIKDTENNEEQIFREWSRNDLSSKNRVELKPYDRS
RFSGNEILITKMGKAPKQCIKTLTYQNISIYKIKTDILGFKYYLKNEGNKPLLHFKKTGPKK
KRKVAV
Amino acid sequence of S. pyogenes 10 Cas9 with His tag
SEQ ID NO: 64
MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTEDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLEKTNRK
VTVKQLKEDYFKKIECFDSVEISGVEDRFNASLGTYHDLLKIIKDKDELDNEENEDILEDIV
LTLTLFEDREMIEERLKTYAHLEDDKVMKQLKRRRYTGWGRLSRKLINGIRDKQSGKTILDF
LKSDGFANRNEMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGDPKKKRKVMDKHHHHHH
Repeat, RNA
SEQ ID NO: 65
GUUUUUGUACUCUCAAGAUUUAAGUAACUAUAAAAC
Repeat, DNA
SEQ ID NO: 66
GTTTTTGTACTCTCAAGATTTAAGTAACTATAAAAC
tracrRNA, RNA
SEQ ID NO: 67
AUAGUUACUUAAAUCUUGCUGAGCCUACAAAGAUAAGGCUUCAUGCCGAAUUCAAGCACCCC
AUCAUUGAUGGGGUGCUUUUCGUAUU
tracrRNA, DNA
SEQ ID NO: 68
ATAGTTACTTAAATCTTGCTGAGCCTACAAAGATAAGGCTTCATGCCGAATTCAAGCACCCC
ATCATTGATGGGGTGCTTTTCGTATT
S. uberis gRNA constant region, RNA
SEQ ID NO: 69
GUUUUUGUACUCUCAAGAUUUCGAAAAAUCUUGCUGAGCCUACAAAGAUAAGGCUUCAUGCC
GAAUUCAAGCACCCCAUCAUUGAUGGGGUGCUUUUCGUAUU
S. uberis gRNA constant region, DNA
SEQ ID NO: 70
GTTTTTGTACTCTCAAGATTTCGAAAAATCTTGCTGAGCCTACAAAGATAAGGCTTCATGCC
GAATTCAAGCACCCCATCATTGATGGGGTGCTTTTCGTATT
S. uberis consensus PAM
SEQ ID NO: 71
AATA
Streptococcus agalactiae dCas9 Amino Acids
(with D10A and H845A underlined)
SEQ ID NO: 193
MNKPYSIGLAIGTNSVGWSIITDDYKVPAKKMRVLGNTDKEYIKKNLIGALLEDGGNTAADR
RLKRTARRRYTRRRNRILYLQEIFAEEMSKVDDSFFHRLEDSFLVEEDKRGSKYPIFATMQE
EKYYHEKFPTIYHLRKELADKKEKADLRLVYLALAHIIKFRGHFLIEDDREDVRNTDIQKQY
QAFLEIFDTTFENNHLLSQNVDVEAILTDKISKSAKKDRILAQYPNQKSTGIFAEFLKLIVG
NQADFKKHFNLEDKTPLQFAKDSYDEDLENLLGQIGDEFADLESVAKKLYDSVLLSGILTVT
DLSTKAPLSASMIQRYDEHHEDLKHLKQFVKASLPENYREVFADSSKDGYAGYIEGKTNQEA
FYKYLLKLLTKQEGSEYFLEKIKNEDFLRKQRTFDNGSIPHQVHLTELRAIIRRQSEYYPEL
KENQDRIEKILTFRIPYYVGPLAREKSDFAWMTRKTDDSIRPWNFEDLVDKEKSAEAFIHRM
TNNDLYLPEEKVLPKHSLIYEKFTVYNELTKVRFLAEGFKDFQFLNRKQKETIFNSLFKEKR
KVTEKDIISFLNKVDGYEGIAIKGIEKQFNASLSTYHDLKKILGKDFLDNTDNELILEDIVQ
TLTLFEDREMIKKCLDIYKDFFTESQLKKLYRRHYTGWGRLSAKLINGIRNKENQKTILDYL
IDDGSANRNEMQLINDDDLSFKPIIDKARTGSHSDNLKEVVGELAGSPAIKKGILQSLKIVD
ELVKVMGYEPEQIVVEMARENQTTAKGLSRSRQRLTTLRESLANLKSNILEEKKPKYVKDQV
ENHHLSDDRLFLYYLQNGRDMYTKKALDIDNLSQYDIDAIIPQAFIKDDSIDNRVLVSSAKN
RGKSDDVPSIEIVKARKMFWKNLLDAKLMSQRKYDNLTKAERGGLTSDDKARFIQRQLVETR
QITKHVARILDERENNEVDNGKKICKVKIVTLKSNLVSNFRKEFGFYKIREVNDYHHAHDAY
LNAVVAKAILTKYPQLEPEFVYGMYRQKKLSKIVHEDKEEKYSEATRKMFFYSNLMNMFKRV
VRLADGSIVVRPVIETGRYMGKTAWDKKKHFATVRKVLSYPQNNIVKKTEIQTGGFSKESIL
AHGNSDKLIPRKTKDIYLDPKKYGGFDSPIVAYSVLVVADIKKGKAQKLKTVTELLGITIME
RSRFEKNPSAFLESKGYLNIRDDKLMILPKYSLFELENGRRRLLASAGELQKGNELALPTQF
MKFLYLASRYNESKGKPEEIEKKQEFVNQHVSYEDDILQLINDESKRVILADANLEKINKLY
QDNKENIPVDELANNIINLFTFTSLGAPAAFKFEDKIVDRKRYTSTKEVLNSTLIHQSITGL
YETRIDLGKLGED
Streptococcus agalactiae dCas9 Nucleotides
(human codon optimized)
SEQ ID NO: 194
ATGAACAAGCCTTATTCAATAGGATTAGCTATAGGGACAAATTCTGTGGGGTGGAGTATAAT
CACCGACGATTACAAGGTGCCTGCAAAGAAGATGCGCGTGCTCGGCAATACAGACAAGGAAT
ATATTAAGAAGAACCTGATCGGGGCCCTCCTTTTTGACGGTGGCAACACAGCAGCTGACCGC
CGCCTCAAGAGGACCGCTCGGAGACGGTATACTCGCCGGCGTAATCGGATCCTGTATTTGCA
GGAAATTTTTGCTGAAGAAATGTCTAAGGTGGATGATTCATTCTTTCACCGGCTCGAAGACT
CCTTTCTGGTGGAGGAAGACAAGAGGGGCTCAAAGTACCCAATCTTCGCCACAATGCAAGAA
GAGAAATACTACCACGAGAAGTTTCCCACAATCTATCATCTCAGGAAAGAGCTGGCCGATAA
AAAAGAGAAGGCCGATTTGCGACTGGTTTACTTGGCCTTGGCACACATCATAAAGTTCCGGG
GACACTTTCTGATTGAAGACGACCGTTTTGACGTCCGCAACACTGATATACAGAAGCAATAC
CAAGCGTTCCTTGAGATCTTTGACACCACATTTGAAAACAACCATCTGCTGAGCCAAAATGT
GGACGTGGAAGCCATTCTGACTGATAAGATCTCTAAATCTGCCAAAAAGGACAGAATCCTTG
CCCAGTACCCCAACCAGAAGTCAACTGGCATTTTCGCCGAGTTTCTGAAGTTGATAGTTGGC
AATCAGGCCGATTTTAAGAAGCACTTCAATTTGGAGGACAAAACGCCTCTCCAATTCGCCAA
GGACTCATATGATGAGGACCTGGAGAATCTGCTTGGCCAAATCGGGGATGAGTTCGCTGATC
TTTTTAGCGTGGCAAAGAAGCTCTATGACTCTGTACTCCTGAGCGGAATCCTGACAGTTACC
GATCTTTCAACAAAGGCACCCCTGAGTGCAAGCATGATTCAACGCTACGACGAGCACCATGA
GGATCTGAAACATCTGAAGCAGTTCGTCAAGGCTTCTCTGCCTGAAAACTATCGGGAGGTCT
TCGCCGACTCATCTAAGGACGGCTACGCCGGATACATCGAGGGAAAGACAAATCAGGAGGCT
TTCTACAAGTACCTGTTGAAGCTGCTTACAAAACAGGAGGGGAGCGAATACTTCCTGGAGAA
GATCAAAAACGAGGACTTCCTGCGTAAACAGAGGACTTTCGATAATGGCTCCATTCCTCACC
AGGTGCATCTCACGGAACTGAGAGCTATCATTAGACGTCAGAGTGAGTATTACCCATTTCTG
AAGGAGAACCAAGACCGAATCGAAAAAATTCTGACGTTCCGGATCCCTTACTATGTCGGACC
TTTAGCTAGGGAAAAAAGTGACTTCGCCTGGATGACCCGAAAGACAGATGATAGTATCAGAC
CATGGAACTTTGAAGACCTGGTGGACAAAGAGAAGAGCGCCGAGGCTTTTATTCACAGGATG
ACCAATAATGATCTCTATCTGCCTGAAGAGAAGGTGCTGCCCAAACACAGTCTCATCTACGA
AAAATTTACAGTCTATAACGAACTGACAAAGGTCCGCTTTCTGGCTGAAGGATTCAAGGACT
TTCAATTTCTGAACCGGAAGCAGAAGGAAACTATCTTTAACTCATTGTTTAAGGAAAAGAGG
AAGGTTACCGAAAAAGACATCATCTCCTTTTTAAACAAGGTAGATGGGTACGAAGGGATTGC
CATTAAAGGCATTGAGAAACAGTTTAACGCCAGCCTTTCAACCTACCATGATCTCAAGAAGA
TCCTCGGAAAAGATTTCCTTGACAATACCGACAACGAACTTATCCTGGAGGATATAGTGCAG
ACACTCACTCTGTTCGAGGACAGGGAAATGATAAAGAAGTGCCTCGACATATATAAAGACTT
CTTTACCGAGAGTCAACTGAAAAAGTTGTATAGAAGGCATTACACCGGTTGGGGCCGACTGA
GTGCAAAACTCATTAACGGCATCCGGAATAAGGAGAATCAAAAGACTATCCTCGATTACCTC
ATCGATGACGGAAGCGCAAACAGAAACTTCATGCAACTCATCAACGATGATGACCTGTCTTT
CAAACCAATTATAGACAAAGCCAGGACTGGGAGCCATAGTGACAATCTGAAGGAAGTGGTGG
GAGAGCTGGCAGGCAGCCCCGCAATTAAGAAGGGGATCCTGCAGAGCCTCAAAATTGTCGAT
GAACTCGTGAAGGTCATGGGCTATGAACCTGAACAGATTGTTGTAGAGATGGCCCGAGAGAA
CCAGACTACTGCGAAGGGACTTAGCCGGAGCAGACAACGACTGACCACTTTGCGAGAGAGTC
TGGCGAACCTGAAGTCTAATATTCTCGAGGAAAAAAAGCCAAAGTACGTGAAGGACCAGGTG
GAGAATCACCACCTGAGCGACGACAGACTCTTTCTGTATTATCTGCAGAACGGCAGAGATAT
GTATACGAAGAAGGCACTGGACATAGACAACCTGAGTCAGTATGACATCGATGCCATTATCC
CTCAGGCCTTCATCAAAGACGATTCAATCGACAATCGCGTACTTGTTAGCAGTGCGAAAAAC
CGGGGAAAGTCTGATGACGTCCCATCCATCGAAATAGTGAAGGCAAGGAAGATGTTCTGGAA
GAATCTGCTGGATGCCAAATTAATGTCACAACGGAAGTACGACAACCTGACAAAGGCAGAAA
GGGGGGGCTTAACAAGCGACGATAAGGCAAGGTTTATCCAGAGGCAGTTGGTCGAGACCAGG
CAAATCACCAAACACGTCGCCCGGATCCTGGATGAACGCTTCAACAATGAAGTCGACAATGG
CAAAAAAATCTGTAAAGTCAAGATAGTGACACTGAAGTCAAATCTGGTGAGCAACTTCCGGA
AAGAATTCGGCTTCTATAAAATTCGCGAAGTGAACGACTATCACCATGCGCACGACGCTTAC
CTGAATGCAGTCGTGGCGAAAGCCATTTTGACCAAGTACCCCCAGCTGGAGCCTGAGTTTGT
GTACGGAATGTACCGACAAAAGAAGCTGAGCAAGATTGTACACGAGGATAAGGAAGAGAAAT
ACTCCGAGGCCACTCGGAAGATGTTCTTCTACTCTAATCTGATGAACATGTTTAAGAGAGTG
GTGAGGTTGGCAGACGGCTCCATTGTTGTAAGGCCAGTGATCGAGACTGGGCGATACATGGG
CAAGACAGCGTGGGACAAGAAGAAGCATTTCGCAACCGTACGGAAAGTCCTGTCCTACCCGC
AGAATAACATTGTGAAGAAGACAGAAATACAAACCGGTGGTTTCTCAAAAGAGTCCATTTTA
GCCCATGGCAACAGTGACAAATTGATTCCACGGAAGACCAAAGATATTTATCTGGACCCTAA
AAAATACGGCGGATTCGACTCACCGATCGTGGCATACAGCGTATTGGTGGTGGCCGATATTA
AGAAGGGTAAAGCCCAGAAACTCAAGACTGTTACCGAGCTCCTGGGTATCACTATAATGGAG
AGAAGCCGGTTTGAGAAGAACCCTAGCGCCTTTTTGGAATCCAAGGGGTATCTGAACATTCG
GGACGATAAGCTGATGATCTTGCCTAAATACAGCCTTTTTGAACTGGAGAATGGACGAAGGC
GCCTGCTTGCCTCAGCGGGGGAACTGCAGAAAGGCAATGAGCTGGCCCTTCCTACCCAGTTC
ATGAAATTTTTGTATCTGGCTAGTAGGTATAACGAGTCAAAAGGCAAGCCAGAGGAGATCGA
AAAGAAGCAGGAATTTGTAAACCAGCATGTGTCATACTTTGATGATATCCTGCAGTTAATCA
ATGACTTCAGTAAACGAGTCATTCTCGCAGACGCCAACTTGGAGAAAATTAATAAGCTGTAC
CAGGACAACAAAGAGAATATACCAGTCGACGAGCTTGCAAATAACATTATTAACCTGTTCAC
TTTTACATCCCTGGGGGCCCCTGCTGCGTTCAAATTTTTCGACAAAATCGTGGATCGAAAGC
GATATACATCCACTAAGGAAGTTCTGAACAGCACTCTCATCCACCAGTCTATCACTGGCCTT
TACGAAACGCGTATTGACTTGGGGAAACTCGGAGAGGAC
Streptococcus agalactiae gRNA scaffold-RNA
SEQ ID NO: 195
GUUUUAGAGCUGUGCGAACACAGCACGUUAAAAUAAGGCAGUGAUUUUUAAUCCAGUCCGUA
UUCAGCUUGAAAAAGUGAGCACCGAUUCGGUGCUUUUUUU
DNA Encoding Streptococcus agalactiae gRNA scaffold
SEQ ID NO: 196
GTTTTAGAGCTGTGCGAACACAGCACGTTAAAATAAGGCAGTGATTTTTAATCCAGTCCGTA
TTCAGCTTGAAAAAGTGAGCACCGATTCGGTGCTTTTTTT
Streptococcus gallolyticus dCas9 Amino Acids
(with D10A and H599A underlined)
SEQ ID NO: 197
MTNGKILGLAIGIASVGVGIIEAKTGKVVHANSRLFSAANAENNAERRGERGSRRLNRRKKH
RVKRVRDLFEKYEIVTDERNLNLNPYELRVKGLTEQLTNEELFAALRTISKRRGISYLDDAE
DDSTGSTDYAKSIDENRRLLKTKTPGQIQLERLEKYGQLRGNFTVYDENGEAHRLINVESTS
DYEKEARKILETQADYNKKITAEFIDDYVEILTQKRKYYHGPGNEKSRTDYGRFRTDGTTLE
NIFGILIGKCSFYPDEYRASKASYTAQEYNFLNDLNNLKVPTETGKLSTEQKEALVEFAKST
ATLGPAKLLKEIAKILDCKVDEIKGYREDDKGKPDLHTFEPYRKLKENLDSVNIDDLSREVL
DKLADILTLNTEREGIEDAIRHNLPNQFTEGQISEIIKVRKSQSTAFNKGWHSFSAKLMNEL
IPELYATSDEQMTILTRLEKFKVNKKSSKNTKTIDEKEVTDEIYNPVVAKSVRQTIKIINAA
VKKYGDFDKIVIEMPRDKNADDEKKFIDKRNKENKKEKDDALKRAAYLYNGTDKLPDEVEHG
NKQLETKIRLWYQQGERCLYSGKPIPIQELVHNSNNFEIDAILPLSLSFDDSLANKVLVYAW
TNQEKGQKTPYQVIDSMDAAWSFREMKDYVLKQKGLGKKKRDYLLTTENIDKIEVKKKFIER
NLVDTRYASRVVLNSLQSALRELGKDTKISVIRGQFTSQLRRKWKIDKSRETYHHHAVDALI
IAASSQLKLWEKQDNPMFVDYGNNQVVDKQTGEILSVSDDEYKELVFQPPYQGFVNMISSKG
FEDEILFSYQVDSKYNRKVSDATIYSTRKAKIGKDKKEETYVLGKIKDIYSQNGFDTFIKKY
NKDKTQFLMYQKDPLTWENVIEVILRDYPTTKKSEDGKNDVKCNPFEEYRRENGLVCKYSKK
GKGTPIKSLKYYDKKLGNCIDITPEGSKNEVVLQSLNPWRADVYFNPETLKYELLGLKYSDL
SFEKGTGKYHISQEKYDVIKEKEGIGKKSEFKFTLYRNDLILIKDTASGEQEIYRFLSRTMP
NVKHYAELKPYDKEKFDNVQELVEALGEADKVGRCIKGLNKSNLSIYKVRTDVLGNKYFVKK
EGDKPKLDFKNNKK
Streptococcus gallolyticus dCas9 Nucleotides (human codon optimized)
SEQ ID NO: 198
ATGACAAACGGCAAAATTCTGGGTCTGGCCATCGGAATCGCTAGCGTTGGCGTGGGAATCAT
TGAAGCGAAGACAGGTAAAGTCGTCCATGCAAATTCTCGATTGTTCTCCGCAGCTAACGCTG
AAAACAATGCGGAGAGAAGGGGTTTCAGAGGCTCTAGGCGGCTCAACCGGCGCAAGAAGCAC
AGGGTAAAAAGAGTGCGAGATCTCTTTGAGAAATATGAGATCGTGACTGATTTTAGAAACCT
GAATCTGAACCCATATGAGCTGAGAGTGAAAGGACTTACGGAACAGCTCACTAATGAAGAGT
TGTTCGCCGCCCTGCGGACCATCAGCAAACGCCGAGGAATTTCCTACCTTGATGACGCCGAA
GATGACAGTACCGGTAGCACAGATTATGCCAAGAGCATTGATGAGAACAGGAGACTGCTGAA
GACTAAGACACCTGGACAGATACAATTGGAACGGCTCGAGAAGTACGGCCAGCTGAGGGGTA
ACTTCACCGTTTATGACGAAAATGGGGAGGCCCATAGACTGATAAATGTGTTCTCAACTTCT
GACTATGAAAAGGAGGCCCGGAAAATCCTCGAGACTCAAGCCGACTACAACAAGAAGATTAC
AGCCGAGTTTATTGACGATTACGTGGAAATTTTAACCCAGAAAAGGAAGTATTACCACGGGC
CAGGAAATGAAAAGAGCCGCACCGACTATGGGAGATTCAGAACGGATGGAACAACCTTAGAG
AATATCTTTGGAATCCTTATTGGTAAATGCTCTTTCTATCCTGACGAGTATCGCGCCAGCAA
AGCCTCCTATACCGCTCAGGAGTACAACTTCTTGAATGATTTGAACAATTTGAAGGTTCCGA
CGGAGACTGGCAAGCTGAGTACCGAGCAAAAGGAGGCCCTTGTGGAATTCGCCAAGTCTACT
GCAACATTAGGTCCTGCTAAACTTCTGAAGGAGATTGCCAAAATTTTGGACTGCAAAGTCGA
TGAAATCAAGGGGTACCGTGAGGATGATAAAGGGAAACCAGACCTGCACACCTTTGAGCCCT
ATAGAAAGTTGAAATTCAATCTGGACAGCGTCAACATTGACGATTTGAGTCGCGAAGTGCTG
GACAAGCTGGCAGACATTTTGACACTTAACACTGAAAGGGAGGGCATTGAGGATGCCATCAG
GCATAACCTGCCCAACCAATTTACTGAGGGCCAGATCTCCGAAATCATCAAGGTGCGCAAAA
GCCAGAGCACTGCTTTCAACAAGGGGTGGCACAGCTTCTCTGCCAAGCTCATGAACGAATTG
ATTCCCGAGCTCTATGCCACAAGCGACGAACAGATGACTATACTTACTCGGCTGGAGAAATT
TAAGGTCAATAAAAAATCCTCCAAAAACACCAAGACGATTGACGAGAAAGAGGTCACTGATG
AAATCTACAATCCAGTTGTAGCCAAGTCTGTCCGGCAAACGATCAAGATCATTAACGCTGCT
GTGAAGAAATATGGAGACTTTGATAAGATTGTGATTGAAATGCCTCGCGACAAGAATGCGGA
CGATGAGAAGAAGTTTATCGATAAGAGAAACAAAGAAAATAAGAAAGAAAAGGATGATGCCC
TGAAGCGGGCAGCTTACCTTTATAATGGAACCGATAAGCTGCCAGATGAGGTGTTTCACGGA
AACAAGCAACTTGAAACCAAGATTCGCCTGTGGTACCAGCAGGGAGAACGGTGTTTGTACTC
AGGCAAGCCTATCCCAATCCAGGAGTTGGTCCACAACTCCAATAACTTCGAAATCGATGCGA
TTCTGCCCCTGTCCCTGAGTTTTGACGACTCCCTGGCCAACAAGGTGCTTGTGTATGCTTGG
ACCAACCAAGAGAAGGGCCAGAAGACGCCCTACCAGGTGATTGATTCTATGGATGCGGCGTG
GTCCTTTCGCGAGATGAAGGACTATGTGCTCAAGCAAAAAGGCCTCGGCAAAAAGAAACGGG
ATTATCTTTTGACCACCGAGAACATTGACAAGATTGAAGTGAAGAAAAAATTCATCGAGCGC
AACTTGGTCGATACCAGATATGCCTCTAGGGTTGTGCTGAACTCACTGCAGTCTGCTTTGAG
AGAGCTGGGTAAAGACACTAAAATTAGTGTAATCAGGGGCCAGTTCACAAGTCAGCTTAGGC
GGAAATGGAAGATCGACAAGTCACGCGAGACATATCATCATCACGCAGTCGACGCACTGATA
ATTGCAGCTTCAAGTCAGCTCAAGTIGTGGGAGAAACAGGATAACCCTATGTTTGTCGACTA
TGGAAACAATCAGGTCGTCGATAAGCAGACCGGGGAAATTTTAAGTGTGTCCGATGACGAGT
ATAAGGAGCTTGTCTTTCAGCCACCGTACCAGGGCTTTGTCAACATGATTAGTAGCAAGGGT
TTTGAGGACGAAATTTTGTTCAGCTACCAGGTCGATTCCAAATACAATAGAAAAGTATCCGA
CGCAACCATATATTCTACTCGCAAGGCCAAGATTGGCAAAGATAAGAAGGAAGAGACCTATG
TATTGGGGAAGATCAAAGACATTTACTCACAAAATGGATTCGACACCTTCATTAAGAAGTAC
AACAAAGATAAGACACAGTTTTTGATGTACCAGAAAGATCCACTGACATGGGAAAACGTGAT
CGAAGTTATACTGCGTGACTACCCCACGACTAAAAAGAGTGAGGACGGAAAAAACGACGTGA
AGTGCAACCCGTTTGAAGAATACCGGAGAGAAAACGGTCTGGTGTGTAAGTACTCTAAGAAA
GGAAAGGGGACCCCTATTAAATCCCTCAAATACTACGACAAAAAACTCGGGAACTGCATCGA
TATCACCCCGGAAGGTTCCAAAAATGAAGTCGTGCTTCAATCCTTGAATCCGTGGAGGGCAG
ATGTGTACTTTAACCCAGAAACCTTGAAGTATGAATTACTGGGACTTAAATACAGTGATCTC
TCATTTGAAAAGGGCACTGGAAAATACCATATCTCTCAGGAGAAGTACGACGTCATTAAGGA
AAAAGAAGGGATCGGGAAAAAATCCGAGTTCAAGTTCACATTGTATAGGAACGACCTGATCC
TTATTAAAGACACAGCCAGCGGTGAGCAGGAGATTTACCGATTTCTGTCTAGAACCATGCCT
AACGTCAAGCACTATGCGGAGCTGAAGCCCTATGACAAAGAAAAATTTGATAACGTCCAGGA
ACTCGTCGAGGCGCTGGGCGAAGCCGACAAGGTAGGCCGCTGTATAAAGGGGCTGAACAAAA
GCAACCTCAGCATCTATAAAGTTAGGACAGATGTGCTCGGGAACAAATACTTCGTTAAGAAG
GAAGGGGACAAGCCCAAGCTGGATTTTAAGAACAATAAAAAG
Streptococcus gallolyticus gRNA scaffold-RNA
SEQ ID NO: 199
GUUUUUGUACUCUCAAGAUUUCGAAAAAUCUUGCUGAGCCUACAAAGAUAAGGCUUUAUGCC
GAAUUCAAGCACCCCAUGUUUUGACAUGGGGUGCUUUU
Streptococcus gallolyticus gRNA scaffold (DNA Encoding)
SEQ ID NO: 200
GTTTTTGTACTCTCAAGATTTCGAAAAATCTTGCTGAGCCTACAAAGATAAGGCTTTATGCC
GAATTCAAGCACCCCATGTTTTGACATGGGGTGCTTTT
Streptococcus iniae dCas9 amino acids (with D10A and H840A underlined)
SEQ ID NO: 201
MRKPYSIGLAIGTNSVGWAVITDDYKVPSKKMRIQGTTDRTSIKKNLIGALLFDNGETAEAT
RLKRTTRRRYTRRKYRIKELQKIFSSEMNELDIAFFPRLSESFLVSDDKEFENHPIFGNLKD
EITYHNDYPTIYHLRQTLADRDQKADLRLIYLALAHIIKERGHFLIEGNLDSENTDVHVLFL
NLVNIYNNLFEEDIVETASIDAEKILTSKTSKSRRLENLIAEIPNQKRNMLFGNLVSLALGL
TPNFKTNFELLEDAKLQISKDSYEEDLDNLLAQIGDQYADLFIAAKKLSDAILLSDIITVKG
ASTKAPLSASMVQRYEEHQQDLALLKNLVKKQIPEKYKEIFDNKEKNGYAGYIDGKTSQEEF
YKYIKPILLKLNGTEKLISKLEREDELRKQRTFDNGSIPHQIHLNELKAIIRRQEKFYPFLK
ENQKKIEKLFTFKIPYYVGPLANGQSSFAWLKRQSNESITPWNFEEVVDQEASARAFIERMT
NFDTYLPEEKVLPKHSPLYEMEMVYNELTKVKYQTEGMKRPVFLSSEDKEEIVNLLFKKDRK
VTVKQLKEEYFSKMKCFHTVTILGVEDRFNASLGTYHDLLKIFKDKAFLDDEANQDILEEIV
WTLTLFEDQAMIERRLVKYADVFEKSVLKKLKKRHYTGWGRLSQKLINGIKDKQTGKTILGF
LKDDGVANRNFMQLINDSSLDFAKIIKHEQEKTIKNESLEETIANLAGSPAIKKGILQSIKI
VDEIVKIMGQNPDNIVIEMARENQSTMQGIKNSRQRLRKLEEVHKNTGSKILKEYNVSNTQL
QSDRLYLYLLQDGKDMYTGKELDYDNLSQYDIDAIIPQSFIKDNSIDNIVLTTQASNRGKSD
NVPNIEIVNKMKSFWYKQLKNGAISQRKFDHLTKAERGALSDEDKAGFIKRQLVETRQITKH
VAQILDSRFNSNLTEDSKSNRNVKIITLKSKMVSDFRKDFGFYKLREVNDYHHAQDAYLNAV
VGTALLKKYPKLEAEFVYGDYKHYDLAKLMIQPDSSLGKATTRMFFYSNLMNFFKKEIKLAD
DTIFTRPQIEVNTETGEIVWDKVKDMQTIRKVMSYPQVNIVMKTEVQTGGFSKESILPKGNS
DKLIARKKSWDPKKYGGFDSPIIAYSVLVVAKIAKGKTQKLKTIKELVGIKIMEQDEFEKDP
IAFLEKKGYQDIQTSSIIKLPKYSLFELENGRKRLLASAKELQKGNELALPNKYVKFLYLAS
HYTKFTGKEEDREKKRSYVESHLYYFDEIMQIIVEYSNRYILADSNLIKIQNLYKEKDNESI
EEQAINMLNLFTFTDLGAPAAFKFENGDIDRKRYSSTNEIINSTLIYQSPTGLYETRIDLSK
LGGK
Streptococcus iniae dCas9 nucleotides (human codon optimized)
SEQ ID NO: 202
ATGCGCAAACCTTACTCAATTGGCCTGGCAATCGGGACTAATTCTGTTGGCTGGGCTGTGAT
TACTGATGATTACAAGGTGCCAAGTAAGAAAATGAGGATTCAGGGCACGACTGATCGGACCA
GCATTAAGAAGAATCTCATTGGGGCCCTCCTGTTCGATAATGGCGAGACTGCCGAGGCCACT
CGATTAAAGAGAACAACAAGGAGGAGGTACACCAGACGGAAGTACCGAATAAAGGAACTGCA
AAAGATCTTCAGCAGCGAAATGAATGAGCTCGACATTGCTTTTTTCCCTAGACTGTCTGAGA
GTTTTCTTGTGAGTGACGACAAAGAATTCGAGAATCATCCGATTTTTGGAAACCTTAAAGAT
GAGATAACTTATCATAACGATTACCCTACTATTTATCACTTGCGACAGACACTTGCAGACCG
TGACCAGAAGGCCGATCTTAGGCTCATTTATCTCGCTCTGGCCCACATTATTAAATTTCGGG
GGCACTTTTTGATCGAAGGCAATCTGGACAGTGAGAACACGGACGTACACGTGCTGTTTCTG
AACCTGGTGAACATATATAATAACCTGTTCGAGGAAGATATAGTTGAAACCGCATCCATAGA
CGCTGAGAAGATTCTTACCTCAAAAACTTCCAAATCCAGGCGGCTCGAGAATCTTATAGCTG
AGATTCCTAACCAGAAGCGGAACATGTTGTTTGGCAACCTCGTGTCTCTGGCTCTCGGCCTG
ACACCAAATTTTAAAACCAATTTTGAGCTGCTGGAGGATGCAAAGTTACAGATCTCCAAGGA
TTCATATGAAGAAGACCTCGACAACTTGTTGGCACAGATTGGGGATCAGTACGCAGATCTCT
TTATCGCCGCTAAAAAGCTTTCTGACGCAATATTACTGTCTGACATCATCACCGTGAAGGGC
GCCTCCACTAAAGCGCCTCTTTCAGCATCCATGGTGCAGAGATATGAAGAGCATCAACAGGA
CCTCGCTCTCCTGAAGAATCTCGTGAAAAAACAGATTCCTGAGAAGTATAAGGAAATCTTCG
ATAACAAGGAGAAGAATGGCTATGCAGGTTATATCGATGGCAAGACCTCCCAGGAGGAATTT
TACAAGTACATCAAGCCCATACTTCTTAAGCTCAACGGCACAGAGAAGTTGATCAGCAAACT
TGAGCGGGAGGACTTCCTGAGAAAGCAACGAACATTCGACAACGGATCTATTCCTCACCAGA
TTCACCTGAATGAGCTCAAGGCAATCATCCGGAGGCAGGAGAAGTTTTATCCCTTTCTGAAG
GAAAATCAGAAGAAAATCGAAAAGCTTTTCACATTTAAAATTCCCTATTACGTCGGGCCACT
CGCCAATGGCCAGAGTAGCTTCGCCTGGCTGAAGAGACAGTCCAACGAGTCTATCACCCCCT
GGAACTTCGAGGAAGTGGTGGATCAAGAGGCCTCAGCGCGCGCCTTCATAGAGAGGATGACT
AACTTCGATACCTATTTACCCGAGGAGAAGGTTCTGCCAAAGCACAGCCCACTCTACGAAAT
GTTTATGGTCTATAATGAGCTCACCAAGGTTAAGTATCAGACCGAGGGGATGAAGAGGCCCG
TCTTTCTCTCTTCCGAAGACAAAGAAGAAATAGTGAATCTCCTGTTCAAAAAAGACCGGAAG
GTCACTGTCAAGCAGCTGAAGGAGGAATATTTCTCCAAAATGAAATGCTTCCACACCGTGAC
AATCTTGGGCGTGGAGGATCGGTTTAATGCTTCTCTGGGCACGTACCATGACCTGCTCAAAA
TTTTTAAAGATAAAGCCTTCTTAGACGATGAGGCCAATCAAGATATCTTGGAAGAGATCGTA
TGGACTTTAACGCTTTTTGAGGATCAAGCCATGATTGAAAGAAGGCTGGTGAAGTACGCGGA
CGTGTTCGAAAAATCCGTCCTTAAAAAGTTAAAGAAACGCCATTACACGGGCTGGGGACGTC
TTTCCCAGAAGCTTATTAATGGGATCAAAGACAAACAAACTGGGAAGACAATTCTCGGCTTT
CTGAAAGACGACGGTGTAGCCAACCGAAATTTTATGCAGTTAATTAACGACAGCTCCCTGGA
CTTCGCAAAGATTATCAAGCATGAACAGGAAAAAACCATCAAGAACGAGTCATTGGAGGAAA
CGATTGCGAACCTGGCAGGCAGCCCCGCCATTAAGAAAGGCATTCTTCAGTCTATTAAAATT
GTCGATGAAATCGTTAAGATTATGGGACAGAACCCAGACAATATTGTTATTGAGATGGCACG
CGAGAACCAATCCACGATGCAAGGAATCAAAAACTCCCGACAGCGTCTGCGCAAGCTCGAGG
AGGTGCATAAGAACACCGGGTCCAAGATTTTGAAAGAATACAACGTGAGTAATACGCAGCTT
CAGAGCGATAGGCTCTATTTATACCTGCTGCAGGACGGAAAGGATATGTACACCGGCAAGGA
GTTGGACTACGACAATCTTAGTCAATATGATATTGATGCGATCATCCCTCAGTCTTTCATAA
AAGATAACTCTATCGACAACATAGTGCTGACTACACAAGCTAGTAATAGGGGCAAGTCAGAC
AACGTGCCCAACATAGAGATTGTGAACAAAATGAAGTCTTTTTGGTATAAACAGCTCAAAAA
TGGGGCAATTAGCCAGCGCAAATTCGACCATTTAACCAAGGCCGAGCGTGGCGCACTGAGCG
ATTTCGATAAGGCAGGCTTTATCAAGCGCCAGCTCGTCGAGACACGGCAGATAACCAAACAT
GTGGCTCAAATCCTGGACAGTCGGTTCAATTCCAATCTTACGGAGGACTCTAAATCTAACAG
AAACGTTAAGATAATAACTCTCAAGTCAAAAATGGTGAGTGACTTCCGAAAGGACTTTGGCT
TTTACAAGCTGAGAGAAGTAAATGATTATCACCACGCCCAGGACGCATATCTCAATGCCGTC
GTCGGTACTGCCTTACTTAAGAAGTACCCTAAACTGGAAGCAGAGTTCGTGTATGGGGATTA
CAAGCACTACGATCTCGCTAAGTTAATGATTCAACCGGACAGTAGCCTTGGAAAAGCCACAA
CCAGAATGTTCTTCTATTCTAACCTCATGAATTTCTTCAAAAAAGAAATCAAACTGGCCGAT
GATACTATATTTACGAGGCCCCAGATTGAAGTGAACACCGAAACTGGGGAGATTGTCTGGGA
TAAGGTAAAGGACATGCAGACCATCAGGAAAGTGATGTCCTATCCACAAGTCAACATAGTGA
TGAAAACCGAAGTCCAGACTGGGGGGTTTTCTAAGGAGAGTATCCTGCCTAAGGGAAACTCA
GACAAACTGATCGCCCGCAAGAAATCCTGGGACCCTAAGAAATACGGTGGTTTCGATAGCCC
TATCATTGCATATTCAGTCCTGGTCGTCGCTAAGATAGCCAAAGGCAAAACCCAGAAACTCA
AGACTATTAAAGAGTTGGTCGGTATCAAAATCATGGAGCAGGACGAATTCGAAAAGGATCCA
ATTGCGTTTCTCGAAAAGAAGGGCTATCAGGACATACAGACCTCTTCCATCATCAAGCTGCC
GAAGTACTCTCTCTTTGAGCTTGAGAATGGACGCAAGAGACTGCTGGCTAGCGCCAAAGAAC
TGCAGAAGGGCAACGAACTGGCCCTCCCTAACAAATACGTAAAGTTCTTGTATTTAGCATCT
CATTACACAAAATTCACAGGTAAGGAGGAAGATCGAGAAAAAAAGCGCTCCTATGTAGAGTC
ACACCTGTATTACTTTGACGAGATTATGCAGATTATCGTTGAGTATTCTAACCGGTACATTC
TCGCCGACAGCAATCTGATTAAAATTCAGAACTTGTACAAAGAGAAGGATAACTTTAGTATC
GAGGAGCAAGCCATTAATATGCTCAATCTCTTCACTTTTACAGATCTCGGCGCGCCAGCCGC
TTTCAAGTTCTTTAACGGAGATATAGATCGGAAGCGGTACAGCTCTACCAACGAGATCATTA
ATTCTACTCTGATTTACCAGAGTCCCACAGGGTTATACGAGACCAGGATCGACCTCAGTAAG
CTGGGGGGCAAA
Streptococcus iniae gRNA scaffold-RNA
SEQ ID NO: 203
GUUUUAGAGCUGUGUUAUUCGAAAACAACACAGCAAGUUAAAAUAAGGCUUGUCCGUAAUCA
ACUUGAAAAAGUGAACACCGAUUCGGUGUUUUUUUAUUUU
Streptococcus iniae gRNA scaffold (DNA encoding)
SEQ ID NO: 204
GTTTTAGAGCTGTGTTATTCGAAAACAACACAGCAAGTTAAAATAAGGCTTGTCCGTAATCA
ACTTGAAAAAGTGAACACCGATTCGGTGTTTTTTTATTTT
Streptococcus lutetiensis dCas9 Amino acids (with D10A and H599A underlined)
SEQ ID NO: 205
MSNGKILGLAIGVASVGVGIIDAKTGNVIHANSRLFSAANAENNAERRGFRGARRLTRRKKH
RVKRVRDLFEKYDISTDERNLNLNPYELRVKGLTEQLTNEELFAALRTIAKRRGISYLDDAE
DDSTGSSDYAKSIDENRRLLKTKTPGQIQLERLEKYGQLRGNFTVYDENGEAHRLINVESTS
DYKNEARKILETQSNYNKQITDEFIEDYIEILTQKRKYYHGPGNEKSRTDYGRFRTDGTTLE
NIFGILIGKCSFYPEEYRASKASYTAQEFNFLNDLNNLKVPTETGKLSTEQKEYLVDFAKKS
KALGASKLLKEIAKIVDCSVDDIKGYRVDNKDKPDLHTFEPYRKLKENLSSIDIDELSRETL
DKLADILTLNTEREGIEDTIKRNLPSQFTEEQISEIVQIRKNQSSAFNKGWHSFSAKLMNEL
IPELYVTSEEQMTILTRLEKFKVNKKSSKNTKTIDEKEITDEIYNPVVAKSVRQTIKIINAA
VKKYGDFDKIVIEMPRDKNAEDEKKFIDKKEKENKKEKDDSLKRAAFLYNGTDNLPDGVFHG
NKELKTKIRLWYQQGERCLYSGKLISIHDLVHNSNKFEIDAILPLSLSFDDSLANKVLVYAW
TNQEKGQKTPYQVIDSMDAAWSFREMKDYVLKQKRLGKKKREYLLTTENIDKIEVKKKFIER
NLVDTRYASRVVLNSLQTALKELGKDTKVSVVRGQFTSQLRRKWNIDKSRETYHHHAVDALI
IAASSQLKLWQKQENPMFESYGENQVVNKETGEILSISDDKYKELVFQPPYQGFVNTISSKG
FEDEILFSYQVDSKFNRKVSDATIYSTRKAKLGKDKKDETYVLGKIKDIYSQDGFDTFIKRY
KKDKTQFLMYQKDPLTWENVIEVILRDYPSEKLSEDGKKTVKCNPFEEYRRENGLICKYSKK
GNGTPIKSLKYYDKKLGNCIDITPEKSKNRVVLRISPWRADIYENLETLKYELMGLKYSDL
SFEKGTGKYHISQEKYDAIREKEGIGKKSEFKFTLYRNDLILIKDTLNNCERMLRFGSKNDT
SKHYVELKPLEKGTFDSEEEILPVLGKVAKSGQFIKGLNKPNISIYKVRTDVLGNKFFIKKE
GDKPKLDFKNNNK
Streptococcus lutetiensis dCas9 Nucleotides (human codon optimized)
SEQ ID NO: 206
ATGTCAAATGGCAAAATCTTAGGCTTGGCCATCGGGGTGGCCAGCGTCGGGGTTGGCATAAT
TGATGCCAAAACCGGCAACGTGATCCACGCAAATAGCAGGCTGTTTAGCGCCGCCAACGCCG
AGAACAATGCTGAGCGGAGGGGATTCCGCGGCGCACGTAGGCTCACGAGGCGCAAAAAACAT
AGAGTGAAGCGGGTCCGTGACCTGTTTGAAAAGTATGATATCTCAACAGATTTCCGCAACTT
AAATCTGAACCCCTACGAGCTCAGGGTGAAAGGCCTGACAGAACAGCTTACCAATGAAGAAC
TCTTCGCAGCTTTAAGAACTATTGCCAAACGGCGCGGCATCTCCTACTTGGATGACGCGGAA
GACGATTCTACCGGAAGCAGCGACTACGCGAAGTCAATCGACGAAAATAGACGTCTTCTGAA
AACCAAAACTCCAGGGCAAATCCAGCTGGAGAGACTGGAGAAGTACGGACAGCTGAGGGGCA
ATTTTACCGTGTATGACGAAAACGGAGAAGCTCACAGACTGATCAATGTTTTTTCCACTTCC
GATTATAAAAACGAAGCCCGGAAGATCCTGGAGACGCAGAGCAACTACAACAAGCAAATCAC
CGATGAGTTCATCGAAGATTACATTGAGATATTAACTCAAAAGCGTAAATACTACCATGGCC
CAGGCAACGAGAAGAGCAGGACCGATTACGGCAGGTTCCGAACAGATGGAACTACCCTGGAG
AACATTTTTGGCATTCTTATTGGAAAATGCTCATTCTATCCAGAGGAATATCGTGCTAGTAA
GGCAAGCTACACCGCCCAAGAATTCAACTTTCTGAATGACCTGAATAATCTGAAGGTCCCCA
CCGAAACGGGCAAGTTATCAACTGAGCAGAAGGAGTATTTAGTGGATTTTGCCAAGAAGTCT
AAGGCTCTGGGAGCGTCTAAGCTTCTGAAGGAGATTGCCAAGATAGTTGATTGCAGCGTTGA
CGACATCAAGGGGTACAGGGTGGATAATAAAGACAAGCCAGATCTGCACACCTTTGAGCCAT
ATAGAAAGTTGAAGTTTAACTTGAGTAGTATCGACATCGATGAACTGTCTAGAGAGACACTC
GACAAACTCGCTGACATTCTTACTCTGAACACAGAACGGGAAGGCATCGAGGATACAATCAA
AAGAAACCTTCCCTCACAGTTTACCGAGGAACAGATAAGCGAGATTGTCCAAATTCGGAAGA
ATCAATCCAGCGCCTTTAACAAGGGTTGGCACTCCTTCTCAGCAAAGTTGATGAACGAGTTA
ATCCCAGAGCTGTACGTGACTTCAGAGGAGCAGATGACAATTCTGACCAGGTTGGAAAAATT
TAAGGTGAACAAGAAGAGCTCCAAAAACACAAAGACCATCGATGAAAAGGAGATTACTGACG
AGATCTATAACCCAGTCGTCGCGAAATCCGTGAGGCAAACTATCAAGATTATCAACGCCGCG
GTGAAAAAGTATGGAGACTTTGACAAAATCGTGATTGAGATGCCACGTGACAAGAATGCAGA
GGATGAGAAAAAATTTATTGACAAAAAGGAGAAGGAAAATAAGAAGGAAAAAGATGATAGCC
TGAAGCGCGCAGCTTTCCTGTATAACGGCACAGACAATTTGCCAGACGGAGTATTTCACGGA
AACAAGGAGCTCAAGACTAAAATTCGCTTATGGTATCAACAAGGCGAGAGGTGCTTGTATAG
CGGCAAACTGATATCCATACACGACCTCGTACACAACAGTAACAAGTTTGAGATTGACGCCA
TCCTTCCACTTAGCCTGAGTTTCGACGACAGCCTGGCAAATAAGGTCTTGGTATATGCTTGG
ACCAATCAGGAGAAGGGGCAAAAAACCCCGTACCAGGTGATAGATAGCATGGACGCGGCATG
GAGTTTTCGGGAAATGAAGGACTACGTTCTCAAACAGAAGAGACTCGGCAAAAAAAAGCGTG
AATACCTGCTGACTACCGAGAACATTGACAAAATCGAAGTCAAAAAAAAGTTCATCGAGCGC
AACCTTGTGGATACCCGCTATGCCTCACGCGTCGTCCTGAACTCTCTGCAGACAGCTCTGAA
AGAACTGGGCAAGGACACCAAAGTGTCTGTCGTTAGGGGTCAATTTACCTCCCAGTTGCGAC
GCAAGTGGAATATCGATAAGTCCAGAGAAACATACCATCATCACGCAGTAGACGCCCTTATC
ATTGCCGCATCTTCTCAGCTTAAACTGTGGCAAAAGCAGGAAAATCCTATGTTTGAGTCTTA
TGGCGAAAATCAGGTCGTCAATAAGGAGACAGGAGAGATCTTATCAATATCCGATGACAAGT
ATAAAGAACTGGTGTTTCAACCACCATACCAAGGGTTTGTCAACACTATCAGCAGTAAAGGC
TTCGAGGATGAGATCTTGTTTTCATATCAGGTGGACAGCAAATTCAACCGGAAAGTTTCTGA
TGCCACCATTTATAGTACTCGCAAAGCGAAACTTGGAAAGGACAAGAAGGATGAGACCTACG
TATTGGGGAAAATCAAGGACATTTACTCTCAGGACGGCTTTGACACCTTCATTAAGCGTTAC
AAAAAGGACAAGACGCAGTTCCTGATGTACCAAAAAGATCCACTGACTTGGGAAAATGTTAT
TGAGGTGATCCTCCGGGATTATCCAAGTGAAAAATTGTCAGAGGACGGCAAAAAAACAGTGA
AGTGCAATCCGTTTGAAGAATATAGGCGAGAGAATGGTCTGATCTGTAAATACTCTAAAAAG
GGCAACGGAACCCCCATCAAGTCCCTGAAATATTACGACAAGAAACTTGGTAACTGCATTGA
CATCACCCCTGAGAAAAGCAAGAACCGCGTGGTGCTGAGGCAGATATCACCTTGGCGCGCTG
ATATCTACTTCAACCTGGAGACCTTGAAATATGAGCTCATGGGCTTGAAATACAGTGACCTG
TCTTTTGAAAAAGGGACCGGGAAGTATCACATTAGCCAGGAAAAGTACGATGCGATTAGAGA
AAAAGAAGGCATTGGCAAAAAGAGCGAGTTTAAGTTTACTTTGTATCGAAACGATCTCATCC
TGATAAAAGATACCCTGAACAATTGTGAGAGGATGCTTAGGTTCGGATCCAAGAACGATACA
TCTAAGCACTACGTGGAACTCAAACCTTTAGAGAAGGGCACCTTTGATTCCGAGGAGGAGAT
CCTTCCAGTGCTGGGCAAGGTTGCGAAATCCGGGCAGTTTATTAAGGGTCTTAACAAACCCA
ATATCTCAATCTATAAGGTGAGGACCGATGTGCTTGGCAACAAATTCTTTATCAAGAAGGAA
GGCGACAAACCCAAGCTGGATTTCAAGAATAATAACAAG
Streptococcus lutetiensis gRNA scaffold-RNA
SEQ ID NO: 207
GUUUUUGUACUCUCAAGCGAACUUGCAGAGCCUACAAAGAUAAGGCUUCAUGCCGAAUUCAA
GCACCCCAUGUUUACAUGGGGUGCUUUUCGUUGUGU
Streptococcus lutetiensis gRNA scaffold (DNA encoding)
SEQ ID NO: 208
GTTTTTGTACTCTCAAGCGAACTTGCAGAGCCTACAAAGATAAGGCTTCATGCCGAATTCAA
GCACCCCATGTTTACATGGGGTGCTTTTCGTTGTGT
Streptococcus mutans dCas9 Amino Acids (with D10A and H840A underlined)
SEQ ID NO: 209
MKKPYSIGLAIGTNSVGWAVVTDDYKVPAKKMKVLGNTDKSHIKKNLLGALLEDSGNTAADR
RLKRTARRRYTRRRNRILYLQEIFAEEMSKVDDSFFHRLEDSELVTEDKRGERHPIFGNLEE
EVKYYENFPTIYHLRQYLADNPEKVDLRLVYLALAHIIKFRGHFLIEGKEDTRNNDVQRLFQ
EFLAVYDNTFENSSLQEQNVQVEEILTDKISKSAKKDRVLKLFPNEKSNGRFAEFLKLIVGN
QADFKKHFELEEKAPLQFSKDTYEEDLEELLGKIGDDYADLFTLAKNLYDAILLSGILTADD
SSTKAPLSASMIQRYNEHQMDLAQLKQFIRQKLSDKYNEVESDVSKDGYAGYIDGKINQEAF
YKYLKGLLNKIEGSGYFLDKIEREDFLRKQRTFDNGSIPHQIHLQEMRAIIRRQAEFYPFLA
DNQDRIEKILTFRIPYYVGPLARGKSDFAWLSRKSADKITPWNFDEIVDKESSAEAFINRMT
NYDLYLPNQKVLPKHSLLYEKFTVYNELTKVKYKTEQGKTAFFDANMKQEIFDGVFKVYRKV
TKDKLMDFLEKEFDEFRIVDLTGLDKENKVFNASYGTYHDLCKILDKDFLDNSKNEKILEDI
VLTLTLFEDREMIRKRLENYSDLLTKEQVKKLERRHYTGWGRLSAELIHGIRNKESRKTILD
YLIDDGNSNRNEMQLINDDALSFKEEIAKAQVIGETDNLNQVVSDIAGSPAIKKGILQSLKI
VDELVKIMGHQPENIVVEMARENQFTNQGRRNSQQRLKGLTDSIKEFGSQILKEHPVENSQL
QNDRLFLYYLQNGRDMYTGEELDIDYLSQYDIDAIIPQAFIKDNSIDNRVLTSSKENRGKSD
DVPSKDVVRKMKPYWSKLLSAKLITQRKFDNLTKAERGGLTDDDKAGFIKRQLVETRQITKH
VARILDERENTETDENNKKIRQVKIVTLKSNLVSNFRKEFELYKVREINDYHHAHDAYLNAV
IGKALLGVYPQLEPEFVYGDYPHFHGHEENKATAKKFFYSNIMNFFKKDDVRTDKNGEIIWK
KDEHISNIKKVLSYPQVNIVKKVEEQTGGFSKESILPKGNSDKLIPRKTKKFYWDTKKYGGF
DSPIVAYSILVIADIEKGKSKKLKTVKALVGVTIMEKMTFERDPVAFLERKGYRNVQEENII
KLPKYSLFKLENGRKRLLASARELQKGNEIVLPNHLGTLLYHAKNIHKVDEPKHLDYVDKHK
DEFKELLDVVSNFSKKYTLAEGNLEKIKELYAQNNGEDLKELASSFINLLTFTAIGAPATFK
FFDKNIDRKRYTSTTEILNATLIHQSITGLYETRIDLSKLGGD
Streptococcus mutans dCas9 Nucleotides (human codon optimized)
SEQ ID NO: 210
ATGAAGAAGCCTTACTCAATTGGCCTGGCTATTGGCACTAATTCAGTGGGATGGGCCGTCGT
TACCGATGATTACAAGGTACCCGCAAAGAAGATGAAGGTCCTTGGTAATACAGATAAAAGTC
ACATAAAGAAGAATCTTCTCGGAGCTCTTCTGTTCGACAGCGGGAACACAGCTGCCGATAGG
CGACTCAAAAGAACTGCTCGCAGGCGCTATACAAGGCGCAGAAACCGCATTCTGTACCTGCA
GGAGATCTTCGCCGAAGAGATGTCCAAAGTGGATGACAGTTTTTTCCATAGGCTCGAGGATA
GCTTCCTGGTGACCGAGGACAAAAGGGGGGAGAGACATCCCATTTTCGGTAATCTTGAAGAG
GAGGTTAAGTACTACGAGAACTTCCCGACTATATATCATCTGCGGCAGTATCTCGCAGACAA
CCCCGAAAAAGTGGACCTGCGACTTGTGTATCTTGCCCTGGCACATATTATAAAATTCAGAG
GCCACTTTCTGATTGAGGGGAAGTTTGATACCCGGAATAATGATGTGCAGCGCCTGTTTCAG
GAATTCTTGGCTGTCTACGACAATACATTTGAGAATAGTAGTTTGCAGGAGCAGAACGTGCA
AGTGGAAGAGATCCTGACAGACAAGATCTCCAAGAGCGCCAAAAAAGATAGGGTGCTCAAAT
TGTTCCCTAATGAGAAATCCAACGGCAGGTTTGCCGAATTCTTGAAACTGATTGTGGGAAAC
CAGGCTGACTTTAAGAAACATTTCGAGCTTGAAGAAAAGGCCCCTTTGCAGTTTTCCAAAGA
CACCTACGAGGAGGATCTGGAGGAACTGCTGGGAAAGATCGGGGATGACTATGCCGATCTGT
TTACCCTCGCCAAGAACCTGTACGATGCGATTCTCTTGTCCGGTATCCTGACGGCAGACGAC
AGTTCAACTAAAGCTCCGCTCTCTGCCAGCATGATTCAGCGATACAATGAGCATCAGATGGA
TCTGGCCCAGCTCAAGCAGTTCATCCGACAGAAACTCAGCGATAAGTACAACGAGGTGTTTA
GCGACGTGTCCAAAGACGGGTACGCAGGCTACATTGACGGCAAGACCAACCAAGAAGCGTTC
TACAAATACCTGAAAGGGCTGCTCAACAAGATAGAAGGATCAGGTTACTTTCTGGATAAAAT
CGAACGGGAGGATTTTTTGCGCAAGCAGCGAACTTTCGACAATGGGTCCATCCCTCATCAGA
TTCACCTGCAGGAAATGAGAGCTATTATTAGGAGACAGGCTGAATTTTACCCTTTTCTGGCA
GATAACCAGGATCGGATCGAGAAAATCTTAACCTTTCGGATCCCATACTATGTGGGCCCACT
GGCCCGTGGCAAATCCGACTTCGCATGGCTGTCACGGAAGTCCGCCGATAAAATTACGCCGT
GGAACTTTGATGAAATTGTCGATAAGGAATCTTCCGCTGAGGCTTTTATCAATCGCATGACC
AATTACGATCTGTACCTGCCTAATCAGAAGGTGTTACCCAAGCATAGCCTGTTGTATGAAAA
ATTCACTGTCTACAATGAACTCACCAAAGTCAAGTACAAGACAGAACAGGGCAAAACCGCCT
TTTTCGACGCTAATATGAAACAGGAAATTTTTGACGGGGTGTTCAAAGTCTATAGAAAGGTC
ACTAAGGACAAACTGATGGATTTTCTGGAGAAGGAATTTGATGAGTTTCGCATAGTTGATCT
TACTGGTTTGGATAAAGAAAATAAGGTCTTCAATGCAAGCTACGGTACATACCACGACCTTT
GTAAAATTCTCGACAAGGATTTCCTCGACAACTCCAAAAATGAAAAGATTCTTGAGGATATC
GTGTTAACCCTGACCCTGTTTGAAGACAGGGAAATGATCCGGAAGCGGCTGGAGAATTACTC
CGACCTGTTGACTAAAGAGCAGGTGAAAAAGCTCGAGAGGCGCCATTACACCGGATGGGGGA
GACTCAGTGCCGAACTTATCCATGGAATTCGAAACAAGGAGAGCAGGAAGACCATTCTCGAT
TATCTGATTGACGATGGTAATAGCAACAGAAATTTTATGCAGCTGATCAACGATGATGCACT
GTCATTTAAGGAGGAAATTGCAAAAGCCCAGGTTATCGGCGAGACCGACAACCTGAATCAGG
TTGTGAGTGACATCGCAGGGAGCCCCGCTATCAAGAAGGGAATCCTCCAGTCCCTCAAGATT
GTCGACGAGCTCGTCAAGATCATGGGGCATCAGCCAGAGAACATTGTCGTGGAGATGGCCCG
CGAAAACCAATTTACCAACCAAGGGAGGCGGAACAGCCAGCAAAGACTGAAGGGCTTAACAG
ATAGCATTAAAGAGTTCGGATCTCAGATACTTAAAGAACACCCCGTCGAAAACTCCCAGTTG
CAGAATGACCGCCTCTTTCTGTATTATCTGCAAAACGGAAGGGACATGTATACGGGAGAGGA
GCTGGATATAGATTACCTTAGTCAATATGATATCGATGCTATAATCCCCCAAGCCTTTATCA
AGGACAACTCTATAGACAATAGGGTCCTGACCTCTAGCAAAGAGAATAGAGGCAAGTCCGAT
GACGTACCTTCTAAGGATGTCGTGCGCAAGATGAAGCCATACTGGAGCAAGCTGCTGTCTGC
AAAGCTTATAACCCAACGAAAGTTCGATAATCTGACTAAGGCCGAGCGCGGCGGGCTGACAG
ATGACGATAAGGCCGGGTTCATTAAGCGCCAGCTGGTGGAAACAAGACAAATCACTAAACAC
GTCGCTCGAATTCTTGATGAGCGGTTTAACACAGAAACGGACGAAAACAACAAAAAGATCCG
CCAGGTAAAAATTGTAACCCTGAAGAGCAACCTTGTTTCTAATTTCAGAAAGGAATTCGAAC
TTTACAAAGTGCGTGAAATCAACGACTACCATCATGCCCATGACGCTTATCTGAACGCTGTC
ATCGGGAAGGCCCTCCTTGGGGTCTATCCTCAGCTGGAGCCTGAATTTGTGTACGGAGATTA
CCCACACTTTCACGGGCACGAAGAGAACAAGGCAACTGCTAAGAAGTTCTTTTATTCAAATA
TCATGAATTTTTTTAAGAAAGACGACGTCAGAACTGATAAAAACGGTGAGATCATTTGGAAG
AAGGACGAACATATCAGTAATATTAAAAAGGTGCTTAGCTATCCTCAGGTGAACATAGTTAA
AAAAGTAGAGGAGCAGACAGGCGGGTTCTCCAAGGAATCCATACTGCCAAAAGGCAACAGCG
ATAAACTGATACCTCGGAAGACTAAAAAATTCTACTGGGATACCAAGAAGTACGGGGGATTT
GACAGCCCCATTGTCGCCTACTCTATATTGGTTATTGCGGACATCGAAAAGGGCAAATCCAA
AAAGCTTAAAACTGTCAAAGCCCTGGTGGGGGTTACCATCATGGAGAAAATGACCTTTGAAC
GCGATCCCGTAGCATTCCTCGAACGCAAGGGCTACCGCAACGTTCAGGAGGAGAATATCATC
AAGTTGCCCAAATATTCTCTCTTTAAGCTGGAGAACGGCAGAAAGCGCCTGCTCGCATCCGC
AAGGGAGTTACAGAAAGGCAACGAAATTGTACTGCCCAATCACCTCGGAACCCTGCTGTATC
ACGCCAAAAATATCCATAAAGTCGACGAACCTAAGCACTTAGACTATGTCGATAAACACAAG
GACGAATTTAAAGAGCTGCTGGACGTGGTTAGCAATTTCTCAAAGAAATACACGCTGGCGGA
AGGTAATCTGGAGAAAATTAAAGAGTTGTACGCTCAGAATAACGGGGAGGATCTTAAAGAAC
TGGCGTCCTCATTTATCAACCTGCTGACCTTCACCGCCATCGGCGCGCCTGCTACATTTAAA
TTCTTCGATAAGAATATCGATAGAAAGAGATATACTTCCACGACCGAAATTTTGAACGCTAC
CCTGATTCACCAATCTATTACCGGGTTATATGAGACACGAATTGATCTGAGCAAACTGGGGG
GGGAT
Streptococcus mutans gRNA scaffold-RNA
SEQ ID NO: 211
GUUUUAGAGCUGUGUCGAAACACAGCAAGUUAAAAUAAGGUUUAUCCGUAUUCAACUUGAAA
AAGUGCGCACCGAUUCGGUGCUUUUUUAUUUGCUUU
Streptococcus mutans gRNA scaffold (DNA Encoding)
SEQ ID NO: 212
GTTTTAGAGCTGTGTCGAAACACAGCAAGTTAAAATAAGGTTTATCCGTATTCAACTTGAAA
AAGTGCGCACCGATTCGGTGCTTTTTTATTTGCTTT
Streptococcus parauberis dCas9 amino acids (with D10A and H840A underlined)
SEQ ID NO: 213
MQKSYSLGLAIGTNSVGWAVITDDYKVPAKKMKVLGNTDRQTVKKNMIGTLLEDSGETAEAR
RLKRTARRRYTRRINRIKYLQSIFDDEMSKIDSAFFQRIKDSFLVPDDKNDDRHPIFGNIKD
EVDYHKNYPTIYHLRKKLADSDEKADLRLIYLALAHIIKFRGHFLIEGDLDSQNTDVNALFL
KLVDTYNLMFEDDKIDTQTIDATVILTEKMSKSRRLENLIAKIPNQKKNTLFGNLISLSLGL
TPNFKANFELSEDAKLQISKDSFEEDLDNLLAQIGDQYADLFIAAKNLSDAILLSDILTVKG
VNTKAPLSASMVQRFNEHQDDLKLLKKLVKVQLPEKYKEIFDIKDKNGYAGYINGKTSQEDF
YKYIKPILSKLKGAESLISKLEREDFLRKQRTFDNGSIPHQIHLNELKSIIRRQEKYYPFLK
DKQVRIEKIFTFRIPYFVGPLANGNSSFAWVKRRSNESITPWNFEEVVEQEASAKVFIERMT
NFDTYLPEEKVLPKHSLLYEMFTVYNELTKVKYQAEGMRKPEFLSSEEKIEIVSNLFKKERK
VTVKQLKENYFNKIRCLDSITISGVEDKFNASLGTYHDLLNIIKNQKILDDEQNQDSLEDIV
LTLTLFEDEKMIAKRLSKYESIFEPSILKKLKKRHYTGWGRLSQKLINGIRDKQTGKTILDF
LIDDGQANRNEMQLINDPSLDFASIIKGAQEKTIKSEKLEETIANLAGSPAIKKGILQSVKI
VDEVVKVMGYEPSNIVIEMARENQSTHRGINNSRERLRKLEEVHKNIGSKILKEHEISNAQL
QSDRVYLYLLQDGKDMYTGKDLDEDRLSQYDIDAIIPQSFIKDNSIDNIVLTSQESNRGKSD
NVPYIAIVNKMKSYWQHQLKSGAISQRKFDNLTKVERGGLSEYDKAGFIKRQLVETRQITKH
VAQILNNRFNNNVDNSSKNKRPVKIITLKSKMVSDFRKEFGFYKIREVNDYHHAHDAYLNAV
VGTALLKKYPKLEAEFVYGDYKHYDLASLVVKSDTSLGKATAKMFFYSNIMNFFKKEVRLAD
GTVITRPQIETNTETGEIVWDKVKDIKTIRKVLSIPQINVVKKTEVQTGGFSKESILPKGDS
DKLIPRKNNWDPKKYGGFDSPIIAYSVLVVAKVAKGKSQKTKSVKELVGITIMEQNEFEKDR
ITFLEKKGYQDIQESLIIKLPKFSLFELENGRKRLLASAKELQKGNELSLPNKYIQFLYLAS
RYTSFSGKEEDREKHRHFVESHLHYFDEIKDIIADESRRYILADANLEKILTLYNEKNQFSI
EEQATNMLNLFTFTGLGAPATLKFFNVDIDRKRYTSSTEILNSTLIRQSITGLYETRIDLSK
IGGD
Streptococcus parauberis dCas9 nucleotides (human codon optimized)
SEQ ID NO: 214
ATGCAAAAGAGCTACTCTCTCGGGTTAGCAATCGGAACAAATAGTGTGGGATGGGCGGTGAT
TACGGACGATTATAAGGTGCCAGCCAAAAAGATGAAGGTTCTTGGCAATACGGACCGGCAGA
CGGTGAAGAAGAACATGATTGGCACTCTGCTGTTTGATAGTGGAGAAACCGCTGAGGCCCGG
AGACTCAAAAGGACTGCTAGGCGACGGTATACGCGGCGTATTAACCGCATTAAATATCTTCA
GTCTATATTTGATGATGAGATGTCAAAGATCGACAGCGCGTTTTTTCAGCGAATTAAAGATT
CCTTCCTTGTCCCAGATGACAAGAATGACGATAGACATCCGATTTTTGGTAACATTAAGGAC
GAGGTTGACTACCATAAGAACTATCCGACAATTTATCACCTGCGCAAGAAGCTGGCAGACTC
CGACGAGAAGGCAGACCTTAGACTGATTTACCTCGCTCTGGCTCACATCATAAAATTTCGAG
GACACTTCTTGATAGAAGGAGATCTCGACAGCCAGAATACTGATGTTAACGCCCTGTTCCTG
AAATTAGTCGACACCTACAACCTCATGTTTGAGGATGACAAAATCGATACGCAGACTATTGA
CGCAACAGTGATTTTAACTGAGAAGATGAGTAAGTCACGGCGACTTGAGAACTTGATAGCCA
AGATACCTAATCAAAAGAAGAATACCCTCTTCGGAAATCTGATTTCACTCAGTCTTGGCCTG
ACACCTAACTTTAAAGCTAATTTTGAATTGAGCGAGGACGCGAAGCTTCAAATCTCTAAGGA
CTCCTTCGAAGAAGATTTGGATAACCTCCTCGCCCAGATCGGTGACCAATACGCTGACCTGT
TTATAGCAGCGAAGAATTTGTCTGACGCTATCCTCCTGTCTGATATCCTTACTGTGAAGGGC
GTGAATACAAAGGCACCCTTATCCGCCAGTATGGTCCAGCGGTTCAACGAACATCAAGACGA
CCTGAAGTTGCTCAAAAAACTCGTGAAGGTGCAACTGCCCGAGAAATACAAAGAAATTTTCG
ACATTAAAGACAAAAATGGGTACGCTGGGTATATTAACGGTAAGACATCCCAGGAGGACTTT
TACAAATATATCAAGCCTATCTTAAGCAAGCTGAAAGGGGCGGAGTCCCTTATCTCTAAATT
GGAGAGAGAAGACTTTTTGCGGAAGCAGAGAACCTTCGATAATGGATCCATTCCCCACCAGA
TTCACTTGAATGAGCTCAAATCCATCATCCGACGACAGGAGAAGTATTATCCCTTTCTGAAG
GATAAACAGGTGCGGATTGAAAAGATCTTCACCTTTAGAATACCATATTTTGTTGGACCATT
GGCTAACGGGAACTCTTCATTTGCTTGGGTTAAGCGAAGATCTAACGAATCTATTACACCAT
GGAACTTTGAGGAAGTCGTTGAGCAGGAGGCCAGCGCCAAGGTCTTCATAGAGCGGATGACT
AATTTTGATACCTACCTGCCAGAGGAGAAGGTCCTTCCCAAGCACTCTTTGCTCTATGAAAT
GTTCACTGTATACAACGAACTGACTAAAGTAAAGTATCAGGCCGAGGGCATGAGAAAGCCCG
AATTCTTGAGTTCAGAAGAAAAGATTGAGATTGTGTCCAACCTGTTTAAGAAGGAGAGAAAG
GTGACAGTCAAGCAGCTTAAGGAAAATTATTTCAATAAGATAAGATGTCTTGACTCAATCAC
CATCAGTGGGGTTGAAGACAAGTTCAACGCATCACTGGGTACTTACCACGATTTACTTAACA
TTATTAAGAACCAGAAGATTCTGGACGATGAGCAGAACCAGGACTCCCTCGAGGATATTGTG
TTGACTCTGACACTGTTCGAGGACGAAAAAATGATCGCGAAGAGGCTGTCAAAGTATGAATC
CATTTTCGAGCCCAGCATTTTGAAGAAATTAAAAAAGCGCCACTATACTGGTTGGGGCCGTT
TATCCCAGAAGCTCATCAACGGCATCCGTGATAAACAGACCGGAAAGACCATCCTGGACTTC
CTGATCGACGATGGCCAGGCGAATCGAAATTTCATGCAATTGATTAACGATCCCTCTCTGGA
CTTTGCGTCAATAATCAAGGGGGCCCAGGAAAAGACGATAAAGAGCGAGAAGCTCGAAGAGA
CCATCGCTAATCTCGCCGGATCTCCCGCTATCAAGAAAGGCATCTTACAGTCTGTGAAGATT
GTAGATGAAGTGGTGAAAGTGATGGGCTATGAACCTAGCAACATTGTCATAGAAATGGCCAG
GGAAAATCAGTCAACCCACCGAGGCATAAATAACTCTAGGGAACGATTACGAAAGCTGGAGG
AGGTCCACAAGAACATTGGCTCCAAGATCTTGAAAGAGCACGAAATTAGCAATGCCCAACTC
CAGAGTGACCGAGTGTACTTGTATCTGTTGCAGGATGGAAAAGATATGTACACCGGTAAGGA
CCTCGATTTCGATCGGCTCTCTCAGTACGATATTGATGCAATCATACCACAGTCCTTTATTA
AGGACAACAGTATTGATAATATCGTCCTGACATCTCAGGAAAGCAATAGAGGAAAGTCAGAT
AATGTGCCCTACATTGCAATCGTGAATAAGATGAAATCATACTGGCAACACCAGCTGAAATC
TGGGGCTATCAGCCAGCGGAAATTTGATAATTTAACTAAGGTGGAGCGGGGCGGCCTCAGCG
AGTATGATAAGGCAGGTTTTATCAAACGTCAGCTCGTTGAGACACGTCAGATAACAAAGCAC
GTGGCACAAATCCTTAATAATAGATTCAACAACAACGTCGATAACAGTAGCAAGAACAAAAG
ACCTGTCAAGATAATCACATTAAAATCTAAAATGGTGTCTGATTTCCGTAAGGAATTCGGCT
TCTATAAAATTAGGGAGGTAAATGACTATCATCACGCCCACGACGCCTACCTCAACGCCGTT
GTCGGGACAGCCCTGTTGAAAAAATATCCAAAGCTGGAGGCAGAATTCGTGTACGGCGATTA
CAAGCACTATGACTTGGCCTCACTGGTTGTCAAGAGCGACACTAGTCTGGGCAAAGCCACTG
CAAAAATGTTTTTTTATTCTAATATCATGAACTTCTTCAAAAAGGAGGTCAGACTGGCAGAT
GGCACCGTGATCACAAGACCTCAGATAGAGACTAATACGGAAACTGGCGAGATCGTGTGGGA
TAAGGTAAAGGACATTAAAACAATTAGGAAGGTGCTGTCTATACCCCAGATCAACGTGGTTA
AAAAGACTGAAGTCCAAACTGGGGGTTTCTCAAAGGAAAGCATCCTGCCCAAGGGCGATAGC
GATAAGCTTATTCCTAGAAAGAACAATTGGGATCCAAAGAAGTATGGTGGCTTTGATTCTCC
GATCATTGCCTATTCTGTCTTAGTGGTCGCAAAAGTGGCGAAGGGCAAAAGCCAGAAGACAA
AGAGTGTCAAAGAACTTGTCGGAATTACTATCATGGAACAGAACGAGTTCGAAAAGGATCGG
ATTACATTCCTTGAGAAAAAAGGATACCAGGATATTCAGGAATCACTGATCATTAAGCTGCC
CAAGTTCAGCTTGTTTGAGCTTGAAAACGGGAGAAAGCGTCTGCTCGCCAGCGCAAAAGAGC
TCCAGAAGGGAAATGAGCTGTCATTGCCAAACAAGTACATCCAATTTTTGTATCTCGCCTCC
AGATATACTAGCTTTAGCGGCAAGGAGGAAGATAGAGAGAAGCACAGACACTTCGTGGAATC
TCACCTGCACTACTTTGATGAGATTAAAGACATAATTGCCGATTTTTCTCGACGCTATATTC
TGGCAGATGCGAACCTTGAAAAAATTCTCACGCTGTACAATGAGAAAAATCAGTTCTCAATT
GAAGAGCAGGCTACCAACATGCTGAACCTCTTCACCTTCACGGGACTGGGAGCCCCTGCCAC
CCTGAAATTTTTCAACGTGGACATTGATCGGAAGCGATACACTTCCTCCACCGAGATTCTGA
ATAGTACCCTCATTAGACAGAGTATTACCGGACTCTACGAGACAAGGATTGACCTCTCCAAA
ATTGGCGGGGAC
Streptococcus parauberis gRNA scaffold-RNA
SEQ ID NO: 215
GUUUUAGAGCUAUGUUAUUCGAAAACAACAUAGCAAGUUAAAAUAAGGCUUGUCCGUAAUCA
ACCUUUCAAGGUGAACACCGAUUCGGUGUUUUUUU
Streptococcus parauberis gRNA scaffold (DNA Encoding)
SEQ ID NO: 216
GTTTTAGAGCTATGTTATTCGAAAACAACATAGCAAGTTAAAATAAGGCTTGTCCGTAATCA
ACCTTTCAAGGTGAACACCGATTCGGTGTTTTTTT
Streptococcus agalactiae dCas9-KRAB amino acids
SEQ ID NO: 217
MNKPYSIGLAIGTNSVGWSIITDDYKVPAKKMRVLGNTDKEYIKKNLIGALLFDGGNTAADR
RLKRTARRRYTRRRNRILYLQEIFAEEMSKVDDSFFHRLEDSFLVEEDKRGSKYPIFATMQE
EKYYHEKFPTIYHLRKELADKKEKADLRLVYLALAHIIKFRGHFLIEDDREDVRNTDIQKQY
QAFLEIFDTTFENNHLLSQNVDVEAILTDKISKSAKKDRILAQYPNQKSTGIFAEFLKLIVG
NQADFKKHFNLEDKTPLQFAKDSYDEDLENLLGQIGDEFADLESVAKKLYDSVLLSGILTVT
DLSTKAPLSASMIQRYDEHHEDLKHLKQFVKASLPENYREVFADSSKDGYAGYIEGKTNQEA
FYKYLLKLLTKQEGSEYFLEKIKNEDFLRKQRTEDNGSIPHQVHLTELRAIIRRQSEYYPEL
KENQDRIEKILTFRIPYYVGPLAREKSDFAWMTRKTDDSIRPWNFEDLVDKEKSAEAFIHRM
TNNDLYLPEEKVLPKHSLIYEKFTVYNELTKVRFLAEGFKDFQFLNRKQKETIFNSLFKEKR
KVTEKDIISFLNKVDGYEGIAIKGIEKQFNASLSTYHDLKKILGKDELDNTDNELILEDIVQ
TLTLFEDREMIKKCLDIYKDFFTESQLKKLYRRHYTGWGRLSAKLINGIRNKENQKTILDYL
IDDGSANRNFMQLINDDDLSFKPIIDKARTGSHSDNLKEVVGELAGSPAIKKGILQSLKIVD
ELVKVMGYEPEQIVVEMARENQTTAKGLSRSRQRLTTLRESLANLKSNILEEKKPKYVKDQV
ENHHLSDDRLFLYYLQNGRDMYTKKALDIDNLSQYDIDAIIPQAFIKDDSIDNRVLVSSAKN
RGKSDDVPSIEIVKARKMFWKNLLDAKLMSQRKYDNLTKAERGGLTSDDKARFIQRQLVETR
QITKHVARILDERENNEVDNGKKICKVKIVTLKSNLVSNFRKEFGFYKIREVNDYHHAHDAY
LNAVVAKAILTKYPQLEPEFVYGMYRQKKLSKIVHEDKEEKYSEATRKMFFYSNLMNMFKRV
VRLADGSIVVRPVIETGRYMGKTAWDKKKHFATVRKVLSYPQNNIVKKTEIQTGGFSKESIL
AHGNSDKLIPRKTKDIYLDPKKYGGFDSPIVAYSVLVVADIKKGKAQKLKTVTELLGITIME
RSRFEKNPSAFLESKGYLNIRDDKLMILPKYSLFELENGRRRLLASAGELQKGNELALPTQF
MKFLYLASRYNESKGKPEEIEKKQEFVNQHVSYEDDILQLINDESKRVILADANLEKINKLY
QDNKENIPVDELANNIINLFTFTSLGAPAAFKFFDKIVDRKRYTSTKEVLNSTLIHQSITGL
YETRIDLGKLGEDTGPKKKRKVASMDAKSLTAWSRTLVTFKDVFVDFTREEWKLLDTAQQIV
YRNVMLENYKNLVSLGYQLTKPDVILRLEKGEEP
Streptococcus gallolyticus dCas9-KRAB amino acids
SEQ ID NO: 218
MTNGKILGLAIGIASVGVGIIEAKTGKVVHANSRLFSAANAENNAERRGFRGSRRLNRRKKH
RVKRVRDLFEKYEIVTDFRNLNLNPYELRVKGLTEQLTNEELFAALRTISKRRGISYLDDAE
DDSTGSTDYAKSIDENRRLLKTKTPGQIQLERLEKYGQLRGNFTVYDENGEAHRLINVESTS
DYEKEARKILETQADYNKKITAEFIDDYVEILTQKRKYYHGPGNEKSRTDYGRFRTDGTTLE
NIFGILIGKCSFYPDEYRASKASYTAQEYNFLNDLNNLKVPTETGKLSTEQKEALVEFAKST
ATLGPAKLLKEIAKILDCKVDEIKGYREDDKGKPDLHTFEPYRKLKENLDSVNIDDLSREVL
DKLADILTLNTEREGIEDAIRHNLPNQFTEGQISEIIKVRKSQSTAFNKGWHSFSAKLMNEL
IPELYATSDEQMTILTRLEKFKVNKKSSKNTKTIDEKEVTDEIYNPVVAKSVRQTIKIINAA
VKKYGDFDKIVIEMPRDKNADDEKKFIDKRNKENKKEKDDALKRAAYLYNGTDKLPDEVFHG
NKQLETKIRLWYQQGERCLYSGKPIPIQELVHNSNNFEIDAILPLSLSFDDSLANKVLVYAW
TNQEKGQKTPYQVIDSMDAAWSFREMKDYVLKQKGLGKKKRDYLLTTENIDKIEVKKKFIER
NLVDTRYASRVVLNSLQSALRELGKDTKISVIRGQFTSQLRRKWKIDKSRETYHHHAVDALI
IAASSQLKLWEKQDNPMFVDYGNNQVVDKQTGEILSVSDDEYKELVFQPPYQGFVNMISSKG
FEDEILFSYQVDSKYNRKVSDATIYSTRKAKIGKDKKEETYVLGKIKDIYSQNGFDTFIKKY
NKDKTQFLMYQKDPLTWENVIEVILRDYPTTKKSEDGKNDVKCNPFEEYRRENGLVCKYSKK
GKGTPIKSLKYYDKKLGNCIDITPEGSKNEVVLQSLNPWRADVYFNPETLKYELLGLKYSDL
SFEKGTGKYHISQEKYDVIKEKEGIGKKSEFKFTLYRNDLILIKDTASGEQEIYRELSRTMP
NVKHYAELKPYDKEKFDNVQELVEALGEADKVGRCIKGLNKSNLSIYKVRTDVLGNKYFVKK
EGDKPKLDFKNNKKTGPKKKRKVASMDAKSLTAWSRTLVTFKDVFVDFTREEWKLLDTAQQI
VYRNVMLENYKNLVSLGYQLTKPDVILRLEKGEEP
Streptococcus iniae dCas9-KRAB amino acids
SEQ ID NO: 219
MRKPYSIGLAIGTNSVGWAVITDDYKVPSKKMRIQGTTDRTSIKKNLIGALLFDNGETAEAT
RLKRTTRRRYTRRKYRIKELQKIFSSEMNELDIAFFPRLSESFLVSDDKEFENHPIFGNLKD
EITYHNDYPTIYHLRQTLADRDQKADLRLIYLALAHIIKFRGHFLIEGNLDSENTDVHVLFL
NLVNIYNNLFEEDIVETASIDAEKILTSKTSKSRRLENLIAEIPNQKRNMLFGNLVSLALGL
TPNFKTNFELLEDAKLQISKDSYEEDLDNLLAQIGDQYADLFIAAKKLSDAILLSDIITVKG
ASTKAPLSASMVQRYEEHQQDLALLKNLVKKQIPEKYKEIFDNKEKNGYAGYIDGKTSQEEF
YKYIKPILLKLNGTEKLISKLEREDFLRKQRTFDNGSIPHQIHLNELKAIIRRQEKFYPFLK
ENQKKIEKLFTFKIPYYVGPLANGQSSFAWLKRQSNESITPWNFEEVVDQEASARAFIERMT
NEDTYLPEEKVLPKHSPLYEMFMVYNELTKVKYQTEGMKRPVFLSSEDKEEIVNLLFKKDRK
VTVKQLKEEYFSKMKCFHTVTILGVEDRFNASLGTYHDLLKIFKDKAFLDDEANQDILEEIV
WTLTLFEDQAMIERRLVKYADVFEKSVLKKLKKRHYTGWGRLSQKLINGIKDKQTGKTILGF
LKDDGVANRNFMQLINDSSLDFAKIIKHEQEKTIKNESLEETIANLAGSPAIKKGILQSIKI
VDEIVKIMGQNPDNIVIEMARENQSTMQGIKNSRQRLRKLEEVHKNTGSKILKEYNVSNTQL
QSDRLYLYLLQDGKDMYTGKELDYDNLSQYDIDAIIPQSFIKDNSIDNIVLTTQASNRGKSD
NVPNIEIVNKMKSFWYKQLKNGAISQRKFDHLTKAERGALSDEDKAGFIKRQLVETRQITKH
VAQILDSRENSNLTEDSKSNRNVKIITLKSKMVSDFRKDFGFYKLREVNDYHHAQDAYLNAV
VGTALLKKYPKLEAEFVYGDYKHYDLAKLMIQPDSSLGKATTRMFFYSNLMNFFKKEIKLAD
DTIFTRPQIEVNTETGEIVWDKVKDMQTIRKVMSYPQVNIVMKTEVQTGGFSKESILPKGNS
DKLIARKKSWDPKKYGGFDSPIIAYSVLVVAKIAKGKTQKLKTIKELVGIKIMEQDEFEKDP
IAFLEKKGYQDIQTSSIIKLPKYSLFELENGRKRLLASAKELQKGNELALPNKYVKFLYLAS
HYTKFTGKEEDREKKRSYVESHLYYFDEIMQIIVEYSNRYILADSNLIKIQNLYKEKDNESI
EEQAINMLNLFTFTDLGAPAAFKFENGDIDRKRYSSTNEIINSTLIYQSPTGLYETRIDLSK
LGGKTGPKKKRKVASMDAKSLTAWSRTLVTFKDVFVDFTREEWKLLDTAQQIVYRNVMLENY
KNLVSLGYQLTKPDVILRLEKGEEP
Streptococcus lutetiensis dCas9-KRAB amino acids
SEQ ID NO: 220
MSNGKILGLAIGVASVGVGIIDAKTGNVIHANSRLFSAANAENNAERRGERGARRLTRRKKH
RVKRVRDLFEKYDISTDERNLNLNPYELRVKGLTEQLTNEELFAALRTIAKRRGISYLDDAE
DDSTGSSDYAKSIDENRRLLKTKTPGQIQLERLEKYGQLRGNFTVYDENGEAHRLINVESTS
DYKNEARKILETQSNYNKQITDEFIEDYIEILTQKRKYYHGPGNEKSRTDYGRFRTDGTTLE
NIFGILIGKCSFYPEEYRASKASYTAQEFNFLNDLNNLKVPTETGKLSTEQKEYLVDFAKKS
KALGASKLLKEIAKIVDCSVDDIKGYRVDNKDKPDLHTFEPYRKLKENLSSIDIDELSRETL
DKLADILTLNTEREGIEDTIKRNLPSQFTEEQISEIVQIRKNQSSAFNKGWHSFSAKLMNEL
IPELYVTSEEQMTILTRLEKFKVNKKSSKNTKTIDEKEITDEIYNPVVAKSVRQTIKIINAA
VKKYGDFDKIVIEMPRDKNAEDEKKFIDKKEKENKKEKDDSLKRAAFLYNGTDNLPDGVFHG
NKELKTKIRLWYQQGERCLYSGKLISIHDLVHNSNKFEIDAILPLSLSFDDSLANKVLVYAW
TNQEKGQKTPYQVIDSMDAAWSFREMKDYVLKQKRLGKKKREYLLTTENIDKIEVKKKFIER
NLVDTRYASRVVLNSLQTALKELGKDTKVSVVRGQFTSQLRRKWNIDKSRETYHHHAVDALI
IAASSQLKLWQKQENPMFESYGENQVVNKETGEILSISDDKYKELVFQPPYQGFVNTISSKG
FEDEILFSYQVDSKFNRKVSDATIYSTRKAKLGKDKKDETYVLGKIKDIYSQDGFDTFIKRY
KKDKTQFLMYQKDPLTWENVIEVILRDYPSEKLSEDGKKTVKCNPFEEYRRENGLICKYSKK
GNGTPIKSLKYYDKKLGNCIDITPEKSKNRVVLRQISPWRADIYENLETLKYELMGLKYSDL
SFEKGTGKYHISQEKYDAIREKEGIGKKSEFKFTLYRNDLILIKDTLNNCERMLRFGSKNDT
SKHYVELKPLEKGTFDSEEEILPVLGKVAKSGQFIKGLNKPNISIYKVRTDVLGNKFFIKKE
GDKPKLDFKNNNKTGPKKKRKVASMDAKSLTAWSRTLVTFKDVFVDFTREEWKLLDTAQQIV
YRNVMLENYKNLVSLGYQLTKPDVILRLEKGEEP
Streptococcus mutans dCas9-KRAB amino acids
SEQ ID NO: 221
MKKPYSIGLAIGTNSVGWAVVTDDYKVPAKKMKVLGNTDKSHIKKNLLGALLEDSGNTAADR
RLKRTARRRYTRRRNRILYLQEIFAEEMSKVDDSFFHRLEDSFLVTEDKRGERHPIFGNLEE
EVKYYENFPTIYHLRQYLADNPEKVDLRLVYLALAHIIKFRGHFLIEGKEDTRNNDVQRLFQ
EFLAVYDNTFENSSLQEQNVQVEEILTDKISKSAKKDRVLKLFPNEKSNGRFAEFLKLIVGN
QADFKKHFELEEKAPLQFSKDTYEEDLEELLGKIGDDYADLFTLAKNLYDAILLSGILTADD
SSTKAPLSASMIQRYNEHQMDLAQLKQFIRQKLSDKYNEVESDVSKDGYAGYIDGKTNQEAF
YKYLKGLLNKIEGSGYFLDKIEREDFLRKQRTFDNGSIPHQIHLQEMRAIIRRQAEFYPFLA
DNQDRIEKILTFRIPYYVGPLARGKSDFAWLSRKSADKITPWNFDEIVDKESSAEAFINRMT
NYDLYLPNQKVLPKHSLLYEKFTVYNELTKVKYKTEQGKTAFFDANMKQEIFDGVEKVYRKV
TKDKLMDFLEKEFDEFRIVDLTGLDKENKVFNASYGTYHDLCKILDKDFLDNSKNEKILEDI
VLTLTLFEDREMIRKRLENYSDLLTKEQVKKLERRHYTGWGRLSAELIHGIRNKESRKTILD
YLIDDGNSNRNEMQLINDDALSFKEEIAKAQVIGETDNLNQVVSDIAGSPAIKKGILQSLKI
VDELVKIMGHQPENIVVEMARENQFTNQGRRNSQQRLKGLTDSIKEFGSQILKEHPVENSQL
QNDRLFLYYLQNGRDMYTGEELDIDYLSQYDIDAIIPQAFIKDNSIDNRVLTSSKENRGKSD
DVPSKDVVRKMKPYWSKLLSAKLITQRKFDNLTKAERGGLTDDDKAGFIKRQLVETRQITKH
VARILDERENTETDENNKKIRQVKIVTLKSNLVSNFRKEFELYKVREINDYHHAHDAYLNAV
IGKALLGVYPQLEPEFVYGDYPHFHGHEENKATAKKFFYSNIMNFFKKDDVRTDKNGEIIWK
KDEHISNIKKVLSYPQVNIVKKVEEQTGGFSKESILPKGNSDKLIPRKTKKFYWDTKKYGGF
DSPIVAYSILVIADIEKGKSKKLKTVKALVGVTIMEKMTFERDPVAFLERKGYRNVQEENII
KLPKYSLFKLENGRKRLLASARELQKGNEIVLPNHLGTLLYHAKNIHKVDEPKHLDYVDKHK
DEFKELLDVVSNFSKKYTLAEGNLEKIKELYAQNNGEDLKELASSFINLLTFTAIGAPATEK
FFDKNIDRKRYTSTTEILNATLIHQSITGLYETRIDLSKLGGDTGPKKKRKVASMDAKSLTA
WSRTLVTFKDVFVDFTREEWKLLDTAQQIVYRNVMLENYKNLVSLGYQLTKPDVILRLEKGE
EP
Streptococcus parauberis dCas9-KRAB amino acids
SEQ ID NO: 222
MQKSYSLGLAIGTNSVGWAVITDDYKVPAKKMKVLGNTDRQTVKKNMIGTLLFDSGETAEAR
RLKRTARRRYTRRINRIKYLQSIFDDEMSKIDSAFFQRIKDSFLVPDDKNDDRHPIFGNIKD
EVDYHKNYPTIYHLRKKLADSDEKADLRLIYLALAHIIKFRGHFLIEGDLDSQNTDVNALFL
KLVDTYNLMFEDDKIDTQTIDATVILTEKMSKSRRLENLIAKIPNQKKNTLFGNLISLSLGL
TPNFKANFELSEDAKLQISKDSFEEDLDNLLAQIGDQYADLFIAAKNLSDAILLSDILTVKG
VNTKAPLSASMVQRFNEHQDDLKLLKKLVKVQLPEKYKEIFDIKDKNGYAGYINGKTSQEDF
YKYIKPILSKLKGAESLISKLEREDELRKQRTEDNGSIPHQIHLNELKSIIRRQEKYYPFLK
DKQVRIEKIFTFRIPYFVGPLANGNSSFAWVKRRSNESITPWNFEEVVEQEASAKVFIERMT
NFDTYLPEEKVLPKHSLLYEMFTVYNELTKVKYQAEGMRKPEFLSSEEKIEIVSNLEKKERK
VTVKQLKENYFNKIRCLDSITISGVEDKENASLGTYHDLLNIIKNQKILDDEQNQDSLEDIV
LTLTLFEDEKMIAKRLSKYESIFEPSILKKLKKRHYTGWGRLSQKLINGIRDKQTGKTILDF
LIDDGQANRNFMQLINDPSLDFASIIKGAQEKTIKSEKLEETIANLAGSPAIKKGILQSVKI
VDEVVKVMGYEPSNIVIEMARENQSTHRGINNSRERLRKLEEVHKNIGSKILKEHEISNAQL
QSDRVYLYLLQDGKDMYTGKDLDEDRLSQYDIDAIIPQSFIKDNSIDNIVLTSQESNRGKSD
NVPYIAIVNKMKSYWQHQLKSGAISQRKFDNLTKVERGGLSEYDKAGFIKRQLVETRQITKH
VAQILNNRFNNNVDNSSKNKRPVKIITLKSKMVSDFRKEFGFYKIREVNDYHHAHDAYLNAV
VGTALLKKYPKLEAEFVYGDYKHYDLASLVVKSDTSLGKATAKMFFYSNIMNFFKKEVRLAD
GTVITRPQIETNTETGEIVWDKVKDIKTIRKVLSIPQINVVKKTEVQTGGFSKESILPKGDS
DKLIPRKNNWDPKKYGGFDSPIIAYSVLVVAKVAKGKSQKTKSVKELVGITIMEQNEFEKDR
ITFLEKKGYQDIQESLIIKLPKFSLFELENGRKRLLASAKELQKGNELSLPNKYIQFLYLAS
RYTSFSGKEEDREKHRHEVESHLHYFDEIKDIIADESRRYILADANLEKILTLYNEKNQFSI
EEQATNMLNLFTFTGLGAPATLKFFNVDIDRKRYTSSTEILNSTLIRQSITGLYETRIDLSK
IGGDTGPKKKRKVASMDAKSLTAWSRTLVTFKDVFVDFTREEWKLLDTAQQIVYRNVMLENY
KNLVSLGYQLTKPDVILRLEKGEEP
Streptococcus parasanguinis Cas9 nuclease, amino acids
SEQ ID NO: 223
MNGLVLGLDIGIASVGVGILKKDIGEIIHTNSRLFSAATADSNIERRGHRGGKRLTRRKKHR
SIRLHDLFEDFGLLTDESKVSINLNPYQLRVQGLDNQLTNEELFIALKNIVKRRGISYLDDA
SEDGGTVSSDYGKAVEENRKLLAEQTPGQIQLDRFEKYGQVRGDENVVENGEKRRLINVETT
SAYSKEAERILRKQQEFNKKITDEFIEDYLTILTGKRKYYHGPGNEKSRTDYGRYTTKKDPE
GKYITLDNIFGILIGKCTFYPDEYRASKASYTAQEFNLLNDLNNLTVPTETKKLSEEQKKTI
IKYAKTAKTLGASTLLKYIAKLIGASVDQIHGYRIDPNKKPEMHTFETYRKMQSLETISVEE
LPRKVLDELAHILTLNTEREGIEEAINATLKDTFSQDQVLELVQFRKNNSSLFSKGWHSFSL
KLMMELIPELYETSEEQMTILTRLGKQKSKETSKRTKYIDEKELTEEIYNPVVAKSVRQAIK
IINEATKKYGIFDNIVIEMARENNEEDAKKEYIKRQKANLDEKNAAMEKAAFQYNGKKELPD
NVFHGHKELATKIRLWHQQGEKCLYTGKNIPISDLIQNQYKYEIDHILPLSLSEDDSLSNKV
LVLATANQEKGQRTPFQALDSMDDAWSYREFKSYVKDSKLLGNKKKEYLLTEEDISKIEVKQ
KFIERNLVDTRYSSRVVLNALQDFYKEHQFDTTISVVRGQFTSQLRRKWGLEKSRETYHHHA
VDALIIAASSQLRLWKKQNNPLISYTEGQFVDQVTGEIISLSDDEYKELVFKAPYDHFVDTL
KSKKFEDSILFSYQVDSKYNRKISDATIYATRKAKLDKENKEYTYTLGKIKDIYALGTKSPS
KTGFYKFLDLYNKDKSQFLMFQKDRKTWDEVIEKIIEQYRPFKEYDENGKEVDENPFEKYRI
ENGPIRKYSKKGNGPEIKSLKYYDNLLGKFVDITPSESKNPVALLSLNPWRTDVYYNTETSK
YEFLGLKYADLCFEKGGAYGISEVKYNKIREKEGIGKESEFKFTLYKNDLILIKDTETNCQQ
IFRFWSRTGKDNPKSFEKHKIELKPYEKARFEKGEELEVLGKVPPSSNQLQKNMQIENLSIY
KVKTDVLGNKHFIKKEGEEPKLKF
Streptococcus parasanguinis Cas9 nuclease, nucleotides
SEQ ID NO: 224
ATGAATGGTTTAGTTTTGGGTTTGGATATTGGTATTGCTTCGGTTGGAGTAGGTATTCTAAA
AAAAGATATTGGTGAAATTATTCACACTAATTCTCGTCTGTTTTCAGCTGCAACGGCTGACA
GTAATATTGAAAGAAGAGGACATAGGGGAGGTAAAAGATTAACTCGCCGGAAGAAACATCGT
AGTATTCGCCTTCATGATTTATTTGAAGACTTTGGTTTGTTAACTGATTTTTCTAAGGTGTC
CATTAATCTAAATCCTTATCAACTCCGTGTACAAGGGTTGGATAATCAATTAACTAATGAAG
AGTTGTTTATTGCTTTAAAGAATATTGTGAAGAGACGTGGTATTAGCTATTTGGATGATGCT
TCTGAGGATGGCGGTACAGTATCGTCTGATTACGGTAAGGCAGTTGAAGAAAATAGAAAATT
ACTTGCTGAACAAACTCCCGGACAAATTCAACTAGATCGTTTCGAAAAATATGGTCAGGTTA
GAGGAGATTTCAATGTTGTAGAAAATGGTGAAAAGCGCAGATTAATCAATGTTTTTACAACA
TCTGCTTATAGCAAAGAAGCTGAAAGAATTCTTAGAAAGCAACAAGAATTCAATAAAAAGAT
TACAGATGAGTTTATAGAGGATTATCTAACAATCCTTACGGGAAAGAGAAAATACTACCATG
GACCAGGTAATGAAAAGTCACGCACCGATTATGGAAGGTATACTACTAAGAAAGATCCTGAA
GGTAAGTACATAACCTTAGATAATATTTTTGGTATTTTAATTGGTAAATGTACATTTTATCC
TGATGAATATAGAGCTTCAAAAGCTTCCTATACTGCTCAAGAGTTCAACTTACTAAATGATT
TAAATAATTTAACTGTTCCAACAGAAACCAAGAAACTGAGTGAGGAACAAAAGAAGACAATT
ATCAAGTATGCGAAGACAGCTAAAACATTAGGAGCTTCAACACTATTGAAGTACATCGCTAA
ATTAATTGGTGCCTCAGTTGATCAGATACATGGATACCGTATTGATCCCAATAAAAAACCTG
AGATGCATACTTTTGAAACCTATCGGAAAATGCAATCATTAGAAACAATCAGCGTGGAAGAA
TTACCGAGAAAGGTCTTAGATGAGCTTGCCCATATTCTAACTTTAAATACAGAGCGAGAGGG
AATAGAAGAAGCAATTAACGCGACGCTAAAAGATACATTTAGTCAGGATCAAGTACTTGAAT
TGGTTCAATTTAGAAAAAATAATAGCAGTCTATTTAGTAAGGGATGGCATAGTTTTTCTCTA
AAACTCATGATGGAGTTGATTCCAGAATTGTATGAGACTTCGGAAGAGCAAATGACAATTCT
TACTCGATTAGGCAAACAAAAATCTAAAGAGACATCAAAACGAACCAAGTATATTGATGAAA
AAGAATTAACAGAAGAAATCTATAACCCTGTGGTAGCAAAATCTGTCAGACAAGCTATAAAG
ATTATCAATGAAGCAACTAAAAAATATGGTATCTTTGATAATATCGTTATTGAAATGGCACG
TGAAAATAATGAGGAAGATGCAAAGAAAGAGTATATCAAGCGTCAAAAAGCAAATCTAGACG
AGAAAAATGCAGCTATGGAAAAAGCGGCTTTCCAATACAATGGGAAAAAAGAATTACCGGAT
AATGTTTTTCATGGGCATAAGGAATTAGCTACTAAGATTCGTTTATGGCATCAGCAAGGAGA
AAAGTGTCTTTACACTGGTAAGAATATACCAATTTCAGATTTGATTCAAAATCAGTATAAGT
ATGAAATTGATCATATTTTACCCTTGTCTCTTTCTTTTGATGATAGTTTATCCAATAAAGTA
TTGGTACTTGCTACAGCGAACCAAGAAAAGGGACAACGTACACCGTTTCAAGCTTTAGATAG
CATGGATGATGCCTGGTCTTACCGTGAGTTTAAATCATATGTAAAAGATTCTAAATTATTAG
GTAATAAAAAGAAGGAATATCTTTTAACGGAAGAAGACATTAGTAAGATTGAGGTGAAACAG
AAATTTATTGAACGAAATTTAGTAGATACTCGTTATTCTTCTCGCGTTGTTCTAAATGCTCT
ACAAGATTTTTATAAGGAGCATCAATTCGATACAACTATTTCAGTGGTACGTGGACAATTTA
CTTCGCAACTCAGAAGAAAGTGGGGACTTGAAAAATCTCGTGAGACCTATCACCATCATGCT
GTAGATGCTTTGATCATTGCTGCATCTAGTCAATTAAGATTATGGAAAAAACAAAATAATCC
TTTGATCTCTTATACAGAAGGTCAATTCGTGGATCAAGTAACTGGTGAAATTATTTCATTGA
GTGACGATGAATATAAAGAGTTAGTATTTAAAGCACCATATGATCATTTTGTTGATACATTG
AAGTCCAAGAAATTTGAGGATAGCATTCTATTTTCTTATCAAGTAGATTCAAAATATAACCG
AAAGATTTCAGATGCAACAATTTACGCAACAAGAAAAGCAAAATTAGATAAAGAGAACAAAG
AATACACTTATACTTTAGGAAAAATAAAAGATATCTATGCTTTAGGGACAAAAAGTCCTTCT
AAAACGGGATTTTATAAATTTTTAGATTTATACAATAAGGATAAATCCCAATTCTTGATGTT
TCAGAAGGATAGAAAAACTTGGGATGAAGTGATAGAGAAGATAATAGAACAATATCGACCAT
TTAAAGAATACGATGAAAATGGAAAAGAAGTTGATTTCAATCCTTTTGAAAAATATAGAATT
GAAAATGGTCCTATTCGCAAATATAGTAAAAAAGGCAATGGTCCAGAAATTAAAAGTCTAAA
ATACTACGATAATCTTTTAGGGAAATTTGTTGATATAACTCCTTCAGAAAGTAAGAATCCTG
TAGCTTTGCTTTCTTTGAATCCTTGGAGAACAGATGTATACTACAATACAGAAACAAGCAAA
TATGAATTCTTAGGATTAAAGTATGCAGATTTGTGTTTCGAAAAAGGTGGTGCTTACGGAAT
TTCAGAAGTTAAATATAATAAAATAAGAGAGAAAGAAGGAATTGGTAAGGAATCTGAATTTA
AGTTTACACTATATAAGAATGATCTAATTTTAATTAAGGATACTGAAACAAATTGCCAACAA
ATCTTTAGATTTTGGTCTCGGACAGGGAAGGATAATCCAAAAAGCTTTGAAAAGCATAAAAT
AGAATTAAAACCTTATGAAAAAGCAAGATTTGAGAAAGGAGAGGAACTTGAGGTATTAGGAA
AGGTACCACCTTCTTCTAATCAATTACAAAAAAATATGCAAATAGAGAATCTTTCTATTTAT
AAAGTTAAAACAGATGTTTTGGGTAATAAACATTTCATCAAAAAAGAGGGTGAAGAACCAAA
ACTCAAATTTTAA
Streptococcus parasanguinis dCas9 amino acids
(with D9A and H604A underlined)
SEQ ID NO: 225
MNGLVLGLAIGIASVGVGILKKDIGEIIHTNSRLFSAATADSNIERRGHRGGKRLTRRKKHR
SIRLHDLFEDFGLLTDESKVSINLNPYQLRVQGLDNQLTNEELFIALKNIVKRRGISYLDDA
SEDGGTVSSDYGKAVEENRKLLAEQTPGQIQLDRFEKYGQVRGDENVVENGEKRRLINVFTT
SAYSKEAERILRKQQEFNKKITDEFIEDYLTILTGKRKYYHGPGNEKSRTDYGRYTTKKDPE
GKYITLDNIFGILIGKCTFYPDEYRASKASYTAQEFNLLNDLNNLTVPTETKKLSEEQKKTI
IKYAKTAKTLGASTLLKYIAKLIGASVDQIHGYRIDPNKKPEMHTFETYRKMQSLETISVEE
LPRKVLDELAHILTLNTEREGIEEAINATLKDTFSQDQVLELVQFRKNNSSLFSKGWHSFSL
KLMMELIPELYETSEEQMTILTRLGKQKSKETSKRTKYIDEKELTEEIYNPVVAKSVRQAIK
IINEATKKYGIFDNIVIEMARENNEEDAKKEYIKRQKANLDEKNAAMEKAAFQYNGKKELPD
NVFHGHKELATKIRLWHQQGEKCLYTGKNIPISDLIQNQYKYEIDAILPLSLSFDDSLSNKV
LVLATANQEKGQRTPFQALDSMDDAWSYREFKSYVKDSKLLGNKKKEYLLTEEDISKIEVKQ
KFIERNLVDTRYSSRVVLNALQDFYKEHQFDTTISVVRGQFTSQLRRKWGLEKSRETYHHHA
VDALIIAASSQLRLWKKQNNPLISYTEGQFVDQVTGEIISLSDDEYKELVFKAPYDHFVDTL
KSKKFEDSILFSYQVDSKYNRKISDATIYATRKAKLDKENKEYTYTLGKIKDIYALGTKSPS
KTGFYKFLDLYNKDKSQFLMFQKDRKTWDEVIEKIIEQYRPFKEYDENGKEVDENPFEKYRI
ENGPIRKYSKKGNGPEIKSLKYYDNLLGKFVDITPSESKNPVALLSLNPWRTDVYYNTETSK
YEFLGLKYADLCFEKGGAYGISEVKYNKIREKEGIGKESEFKFTLYKNDLILIKDTETNCQQ
IFRFWSRTGKDNPKSFEKHKIELKPYEKARFEKGEELEVLGKVPPSSNQLQKNMQIENLSIY
KVKTDVLGNKHFIKKEGEEPKLKF
Streptococcus parasanguinis dCas9 nucleotides
SEQ ID NO: 226
ATGAACGGACTGGTTTTGGGTCTTGCCATCGGGATCGCTAGCGTTGGGGTGGGCATCCTGAA
GAAGGACATAGGAGAAATCATTCATACCAATTCTAGACTGTTTTCAGCTGCCACAGCCGACT
CTAATATTGAACGACGAGGACATCGTGGCGGCAAGAGGCTGACAAGACGAAAAAAACACCGA
AGCATACGACTTCACGATCTTTTCGAGGATTTTGGACTGCTGACGGACTTTTCAAAAGTTTC
CATCAACTTGAATCCGTACCAGTTACGCGTACAGGGTCTGGACAACCAGCTGACAAACGAGG
AGCTGTTTATCGCTCTTAAGAATATCGTGAAGCGCCGGGGGATTAGCTACTTAGACGATGCC
TCTGAAGACGGCGGAACCGTGTCTTCTGATTATGGGAAGGCTGTCGAAGAAAATAGAAAACT
CTTAGCCGAACAGACTCCTGGGCAGATCCAGCTGGACAGATTCGAAAAGTACGGCCAAGTCC
GAGGCGACTTCAATGTCGTGGAGAACGGTGAGAAACGACGTCTGATTAACGTCTTTACAACT
AGCGCCTATTCCAAGGAGGCCGAGAGGATACTGAGGAAGCAGCAGGAGTTCAACAAGAAAAT
AACGGATGAGTTCATCGAGGATTACCTGACCATTCTTACTGGAAAAAGAAAATATTACCATG
GTCCTGGAAACGAAAAGTCCCGGACCGATTACGGGCGGTACACAACCAAAAAGGACCCAGAG
GGCAAATACATCACCCTCGATAATATTTTCGGGATCCTCATCGGTAAATGCACTTTTTACCC
CGATGAGTATCGCGCGTCTAAAGCTTCATATACCGCACAGGAGTTCAATCTGCTCAACGACC
TGAATAACCTGACCGTGCCTACCGAAACCAAAAAACTGTCAGAGGAGCAGAAGAAGACGATA
ATAAAATACGCCAAAACGGCTAAGACCCTTGGCGCTTCTACTCTGCTGAAGTATATAGCCAA
ACTGATCGGTGCTTCCGTTGACCAGATTCACGGGTATAGAATCGACCCAAATAAAAAGCCCG
AAATGCACACCTTCGAGACGTACCGGAAAATGCAATCCCTGGAGACGATCTCAGTGGAGGAA
CTGCCTCGCAAAGTGCTTGACGAACTCGCCCATATTCTGACATTGAACACTGAGCGCGAAGG
CATCGAGGAGGCTATTAATGCCACCTTGAAAGATACGTTTAGTCAGGACCAGGTCCTCGAAC
TCGTGCAGTTCCGCAAAAATAACTCTTCCTTATTCTCAAAGGGATGGCATAGTTTCAGCCTG
AAACTGATGATGGAACTGATTCCTGAACTCTATGAGACTAGTGAAGAGCAGATGACTATACT
GACTCGTCTGGGGAAACAGAAGTCTAAGGAGACAAGTAAACGAACTAAGTACATTGATGAAA
AGGAGCTGACAGAAGAGATTTATAATCCAGTCGTGGCTAAATCCGTCCGTCAGGCTATTAAG
ATCATTAACGAGGCAACGAAAAAGTACGGAATCTTCGATAACATTGTGATCGAAATGGCCCG
TGAGAACAATGAAGAAGATGCTAAGAAGGAGTACATCAAGCGGCAGAAGGCAAACTTGGATG
AGAAGAACGCCGCAATGGAGAAAGCTGCTTTTCAATACAACGGTAAGAAGGAACTCCCGGAT
AACGTCTTCCACGGCCATAAGGAGCTCGCCACAAAAATACGGTTGTGGCACCAGCAGGGGGA
AAAGTGCCTCTACACTGGAAAAAATATCCCTATCTCCGACCTTATTCAAAATCAGTACAAGT
ATGAAATCGACGCCATCCTCCCACTGTCCCTCAGTTTCGACGATAGCCTGTCCAACAAGGTC
CTCGTGCTGGCTACCGCCAATCAGGAAAAGGGCCAAAGAACTCCTTTTCAAGCTCTCGATTC
AATGGATGACGCCTGGAGCTATCGGGAGTTCAAATCCTATGTGAAGGATTCTAAACTCTTGG
GGAATAAAAAGAAGGAATACTTGTTAACAGAGGAGGATATCAGTAAGATCGAGGTGAAACAG
AAGTTTATCGAACGGAATTTAGTTGACACAAGGTACTCAAGTCGCGTGGTTCTTAACGCCCT
TCAGGACTTCTACAAGGAACACCAGTTCGATACCACCATTAGCGTGGTTAGAGGACAATTTA
CATCCCAGCTGCGGCGGAAATGGGGACTGGAGAAGAGCCGAGAAACCTACCATCACCACGCC
GTGGACGCGCTGATTATTGCCGCCAGTAGCCAGCTCCGCCTCTGGAAAAAACAGAACAATCC
TCTTATCAGTTACACCGAAGGCCAGTTTGTGGATCAGGTGACCGGCGAGATTATATCCCTTT
CTGACGACGAATACAAAGAGCTGGTTTTCAAAGCTCCATATGACCACTTTGTTGACACACTG
AAGTCAAAGAAGTTTGAGGATTCTATCCTCTTTTCATACCAGGTGGATTCTAAGTACAACCG
GAAGATTAGCGACGCAACCATATATGCAACTCGTAAAGCCAAATTGGACAAGGAGAATAAAG
AGTATACTTACACTCTGGGTAAAATCAAAGATATCTATGCTCTTGGCACGAAAAGCCCTTCT
AAAACCGGCTTTTACAAGTTTCTGGACCTGTATAATAAAGACAAGTCTCAGTTCCTGATGTT
TCAGAAAGATCGCAAGACCTGGGACGAGGTGATTGAAAAGATCATTGAGCAATACCGGCCTT
TTAAAGAATACGACGAAAACGGAAAAGAAGTAGACTTCAACCCTTTTGAAAAGTACCGGATC
GAAAATGGCCCCATCCGAAAATATTCCAAAAAGGGCAATGGCCCAGAAATAAAGAGTCTCAA
GTATTACGATAACTTACTGGGTAAGTTTGTGGATATCACACCTTCAGAGTCCAAGAACCCCG
TAGCTCTGCTGTCCCTGAACCCGTGGAGGACTGACGTCTACTACAACACCGAAACATCCAAG
TATGAGTTCCTTGGGCTGAAGTATGCTGATTTGTGCTTCGAAAAGGGGGGTGCATATGGTAT
CTCTGAGGTGAAATACAATAAAATAAGAGAGAAAGAAGGTATCGGAAAGGAGTCTGAATTCA
AATTTACCCTTTATAAGAATGACCTGATTCTGATCAAAGATACAGAGACAAACTGCCAACAG
ATTTTTCGCTTCTGGTCTCGAACTGGAAAAGACAATCCTAAATCATTTGAGAAGCATAAAAT
TGAGCTTAAACCGTATGAAAAGGCTCGTTTCGAGAAGGGTGAAGAACTGGAGGTGCTGGGAA
AGGTGCCACCTAGTTCAAACCAACTGCAGAAAAATATGCAAATTGAGAACCTCTCCATCTAC
AAGGTGAAGACTGATGTGCTTGGCAATAAGCATTTTATCAAAAAGGAAGGAGAAGAACCTAA
GCTCAAATTT
Streptococcus parasanguinis dCas9-KRAB amino acids
SEQ ID NO: 227
MNGLVLGLAIGIASVGVGILKKDIGEIIHTNSRLFSAATADSNIERRGHRGGKRLTRRKKHR
SIRLHDLFEDFGLLTDESKVSINLNPYQLRVQGLDNQLTNEELFIALKNIVKRRGISYLDDA
SEDGGTVSSDYGKAVEENRKLLAEQTPGQIQLDRFEKYGQVRGDENVVENGEKRRLINVETT
SAYSKEAERILRKQQEFNKKITDEFIEDYLTILTGKRKYYHGPGNEKSRTDYGRYTTKKDPE
GKYITLDNIFGILIGKCTFYPDEYRASKASYTAQEFNLLNDLNNLTVPTETKKLSEEQKKTI
IKYAKTAKTLGASTLLKYIAKLIGASVDQIHGYRIDPNKKPEMHTFETYRKMQSLETISVEE
LPRKVLDELAHILTLNTEREGIEEAINATLKDTFSQDQVLELVQFRKNNSSLFSKGWHSFSL
KLMMELIPELYETSEEQMTILTRLGKQKSKETSKRTKYIDEKELTEEIYNPVVAKSVRQAIK
IINEATKKYGIFDNIVIEMARENNEEDAKKEYIKRQKANLDEKNAAMEKAAFQYNGKKELPD
NVFHGHKELATKIRLWHQQGEKCLYTGKNIPISDLIQNQYKYEIDAILPLSLSFDDSLSNKV
LVLATANQEKGQRTPFQALDSMDDAWSYREFKSYVKDSKLLGNKKKEYLLTEEDISKIEVKQ
KFIERNLVDTRYSSRVVLNALQDFYKEHQFDTTISVVRGQFTSQLRRKWGLEKSRETYHHHA
VDALIIAASSQLRLWKKQNNPLISYTEGQFVDQVTGEIISLSDDEYKELVFKAPYDHFVDTL
KSKKFEDSILFSYQVDSKYNRKISDATIYATRKAKLDKENKEYTYTLGKIKDIYALGTKSPS
KTGFYKFLDLYNKDKSQFLMFQKDRKTWDEVIEKIIEQYRPFKEYDENGKEVDENPFEKYRI
ENGPIRKYSKKGNGPEIKSLKYYDNLLGKFVDITPSESKNPVALLSLNPWRTDVYYNTETSK
YEFLGLKYADLCFEKGGAYGISEVKYNKIREKEGIGKESEFKFTLYKNDLILIKDTETNCQQ
IFRFWSRTGKDNPKSFEKHKIELKPYEKARFEKGEELEVLGKVPPSSNQLQKNMQIENLSIY
KVKTDVLGNKHFIKKEGEEPKLKFTGPKKKRKVASMDAKSLTAWSRTLVTFKDVFVDFTREE
WKLLDTAQQIVYRNVMLENYKNLVSLGYQLTKPDVILRLEKGEEP
Streptococcus parasanguinis dCas9-KRAB nucleotides
SEQ ID NO: 228
ATGAACGGACTGGTTTTGGGTCTTGCCATCGGGATCGCTAGCGTTGGGGTGGGCATCCTGAA
GAAGGACATAGGAGAAATCATTCATACCAATTCTAGACTGTTTTCAGCTGCCACAGCCGACT
CTAATATTGAACGACGAGGACATCGTGGCGGCAAGAGGCTGACAAGACGAAAAAAACACCGA
AGCATACGACTTCACGATCTTTTCGAGGATTTTGGACTGCTGACGGACTTTTCAAAAGTTTC
CATCAACTTGAATCCGTACCAGTTACGCGTACAGGGTCTGGACAACCAGCTGACAAACGAGG
AGCTGTTTATCGCTCTTAAGAATATCGTGAAGCGCCGGGGGATTAGCTACTTAGACGATGCC
TCTGAAGACGGCGGAACCGTGTCTTCTGATTATGGGAAGGCTGTCGAAGAAAATAGAAAACT
CTTAGCCGAACAGACTCCTGGGCAGATCCAGCTGGACAGATTCGAAAAGTACGGCCAAGTCC
GAGGCGACTTCAATGTCGTGGAGAACGGTGAGAAACGACGTCTGATTAACGTCTTTACAACT
AGCGCCTATTCCAAGGAGGCCGAGAGGATACTGAGGAAGCAGCAGGAGTTCAACAAGAAAAT
AACGGATGAGTTCATCGAGGATTACCTGACCATTCTTACTGGAAAAAGAAAATATTACCATG
GTCCTGGAAACGAAAAGTCCCGGACCGATTACGGGCGGTACACAACCAAAAAGGACCCAGAG
GGCAAATACATCACCCTCGATAATATTTTCGGGATCCTCATCGGTAAATGCACTTTTTACCC
CGATGAGTATCGCGCGTCTAAAGCTTCATATACCGCACAGGAGTTCAATCTGCTCAACGACC
TGAATAACCTGACCGTGCCTACCGAAACCAAAAAACTGTCAGAGGAGCAGAAGAAGACGATA
ATAAAATACGCCAAAACGGCTAAGACCCTTGGCGCTTCTACTCTGCTGAAGTATATAGCCAA
ACTGATCGGTGCTTCCGTTGACCAGATTCACGGGTATAGAATCGACCCAAATAAAAAGCCCG
AAATGCACACCTTCGAGACGTACCGGAAAATGCAATCCCTGGAGACGATCTCAGTGGAGGAA
CTGCCTCGCAAAGTGCTTGACGAACTCGCCCATATTCTGACATTGAACACTGAGCGCGAAGG
CATCGAGGAGGCTATTAATGCCACCTTGAAAGATACGTTTAGTCAGGACCAGGTCCTCGAAC
TCGTGCAGTTCCGCAAAAATAACTCTTCCTTATTCTCAAAGGGATGGCATAGTTTCAGCCTG
AAACTGATGATGGAACTGATTCCTGAACTCTATGAGACTAGTGAAGAGCAGATGACTATACT
GACTCGTCTGGGGAAACAGAAGTCTAAGGAGACAAGTAAACGAACTAAGTACATTGATGAAA
AGGAGCTGACAGAAGAGATTTATAATCCAGTCGTGGCTAAATCCGTCCGTCAGGCTATTAAG
ATCATTAACGAGGCAACGAAAAAGTACGGAATCTTCGATAACATTGTGATCGAAATGGCCCG
TGAGAACAATGAAGAAGATGCTAAGAAGGAGTACATCAAGCGGCAGAAGGCAAACTTGGATG
AGAAGAACGCCGCAATGGAGAAAGCTGCTTTTCAATACAACGGTAAGAAGGAACTCCCGGAT
AACGTCTTCCACGGCCATAAGGAGCTCGCCACAAAAATACGGTTGTGGCACCAGCAGGGGGA
AAAGTGCCTCTACACTGGAAAAAATATCCCTATCTCCGACCTTATTCAAAATCAGTACAAGT
ATGAAATCGACGCCATCCTCCCACTGTCCCTCAGTTTCGACGATAGCCTGTCCAACAAGGTC
CTCGTGCTGGCTACCGCCAATCAGGAAAAGGGCCAAAGAACTCCTTTTCAAGCTCTCGATTC
AATGGATGACGCCTGGAGCTATCGGGAGTTCAAATCCTATGTGAAGGATTCTAAACTCTTGG
GGAATAAAAAGAAGGAATACTTGTTAACAGAGGAGGATATCAGTAAGATCGAGGTGAAACAG
AAGTTTATCGAACGGAATTTAGTTGACACAAGGTACTCAAGTCGCGTGGTTCTTAACGCCCT
TCAGGACTTCTACAAGGAACACCAGTTCGATACCACCATTAGCGTGGTTAGAGGACAATTTA
CATCCCAGCTGCGGCGGAAATGGGGACTGGAGAAGAGCCGAGAAACCTACCATCACCACGCC
GTGGACGCGCTGATTATTGCCGCCAGTAGCCAGCTCCGCCTCTGGAAAAAACAGAACAATCC
TCTTATCAGTTACACCGAAGGCCAGTTTGTGGATCAGGTGACCGGCGAGATTATATCCCTTT
CTGACGACGAATACAAAGAGCTGGTTTTCAAAGCTCCATATGACCACTTTGTTGACACACTG
AAGTCAAAGAAGTTTGAGGATTCTATCCTCTTTTCATACCAGGTGGATTCTAAGTACAACCG
GAAGATTAGCGACGCAACCATATATGCAACTCGTAAAGCCAAATTGGACAAGGAGAATAAAG
AGTATACTTACACTCTGGGTAAAATCAAAGATATCTATGCTCTTGGCACGAAAAGCCCTTCT
AAAACCGGCTTTTACAAGTTTCTGGACCTGTATAATAAAGACAAGTCTCAGTTCCTGATGTT
TCAGAAAGATCGCAAGACCTGGGACGAGGTGATTGAAAAGATCATTGAGCAATACCGGCCTT
TTAAAGAATACGACGAAAACGGAAAAGAAGTAGACTTCAACCCTTTTGAAAAGTACCGGATC
GAAAATGGCCCCATCCGAAAATATTCCAAAAAGGGCAATGGCCCAGAAATAAAGAGTCTCAA
GTATTACGATAACTTACTGGGTAAGTTTGTGGATATCACACCTTCAGAGTCCAAGAACCCCG
TAGCTCTGCTGTCCCTGAACCCGTGGAGGACTGACGTCTACTACAACACCGAAACATCCAAG
TATGAGTTCCTTGGGCTGAAGTATGCTGATTTGTGCTTCGAAAAGGGGGGTGCATATGGTAT
CTCTGAGGTGAAATACAATAAAATAAGAGAGAAAGAAGGTATCGGAAAGGAGTCTGAATTCA
AATTTACCCTTTATAAGAATGACCTGATTCTGATCAAAGATACAGAGACAAACTGCCAACAG
ATTTTTCGCTTCTGGTCTCGAACTGGAAAAGACAATCCTAAATCATTTGAGAAGCATAAAAT
TGAGCTTAAACCGTATGAAAAGGCTCGTTTCGAGAAGGGTGAAGAACTGGAGGTGCTGGGAA
AGGTGCCACCTAGTTCAAACCAACTGCAGAAAAATATGCAAATTGAGAACCTCTCCATCTAC
AAGGTGAAGACTGATGTGCTTGGCAATAAGCATTTTATCAAAAAGGAAGGAGAAGAACCTAA
GCTCAAATTTACCGGTCCTAAGAAAAAGCGGAAAGTGGCTAGCATGGATGCTAAGTCACTAA
CTGCCTGGTCCCGGACACTGGTGACCTTCAAGGATGTATTTGTGGACTTCACCAGGGAGGAG
TGGAAGCTGCTGGACACTGCTCAGCAGATCGTGTACAGAAATGTGATGCTGGAGAACTATAA
GAACCTGGTTTCCTTGGGTTATCAGCTTACTAAGCCAGATGTGATCCTCCGGTTGGAGAAGG
GAGAAGAGCCC
Streptococcus parasanguinis dCas9-p300 amino acids
SEQ ID NO: 229
MNGLVLGLAIGIASVGVGILKKDIGEIIHTNSRLFSAATADSNIERRGHRGGKRLTRRKKHR
SIRLHDLFEDFGLLTDESKVSINLNPYQLRVQGLDNQLTNEELFIALKNIVKRRGISYLDDA
SEDGGTVSSDYGKAVEENRKLLAEQTPGQIQLDRFEKYGQVRGDENVVENGEKRRLINVETT
SAYSKEAERILRKQQEFNKKITDEFIEDYLTILTGKRKYYHGPGNEKSRTDYGRYTTKKDPE
GKYITLDNIFGILIGKCTFYPDEYRASKASYTAQEFNLLNDLNNLTVPTETKKLSEEQKKTI
IKYAKTAKTLGASTLLKYIAKLIGASVDQIHGYRIDPNKKPEMHTFETYRKMQSLETISVEE
LPRKVLDELAHILTLNTEREGIEEAINATLKDTFSQDQVLELVQFRKNNSSLFSKGWHSFSL
KLMMELIPELYETSEEQMTILTRLGKQKSKETSKRTKYIDEKELTEEIYNPVVAKSVRQAIK
IINEATKKYGIFDNIVIEMARENNEEDAKKEYIKRQKANLDEKNAAMEKAAFQYNGKKELPD
NVFHGHKELATKIRLWHQQGEKCLYTGKNIPISDLIQNQYKYEIDAILPLSLSEDDSLSNKV
LVLATANQEKGQRTPFQALDSMDDAWSYREFKSYVKDSKLLGNKKKEYLLTEEDISKIEVKQ
KFIERNLVDTRYSSRVVLNALQDFYKEHQFDTTISVVRGQFTSQLRRKWGLEKSRETYHHHA
VDALIIAASSQLRLWKKQNNPLISYTEGQFVDQVTGEIISLSDDEYKELVFKAPYDHEVDTL
KSKKFEDSILFSYQVDSKYNRKISDATIYATRKAKLDKENKEYTYTLGKIKDIYALGTKSPS
KTGFYKFLDLYNKDKSQFLMFQKDRKTWDEVIEKIIEQYRPFKEYDENGKEVDENPFEKYRI
ENGPIRKYSKKGNGPEIKSLKYYDNLLGKFVDITPSESKNPVALLSLNPWRIDVYYNTETSK
YEFLGLKYADLCFEKGGAYGISEVKYNKIREKEGIGKESEFKFTLYKNDLILIKDTETNCQQ
IFRFWSRTGKDNPKSFEKHKIELKPYEKARFEKGEELEVLGKVPPSSNQLQKNMQIENLSIY
KVKTDVLGNKHFIKKEGEEPKLKFTGPKKKRKVASIFKPEELRQALMPTLEALYRQDPESLP
FRQPVDPQLLGIPDYFDIVKSPMDLSTIKRKLDTGQYQEPWQYVDDIWLMENNAWLYNRKTS
RVYKYCSKLSEVFEQEIDPVMQSLGYCCGRKLEFSPQTLCCYGKQLCTIPRDATYYSYQNRY
HFCEKCFNEIQGESVSLGDDPSQPQTTINKEQFSKRKNDTLDPELFVECTECGRKMHQICVL
HHEIIWPAGFVCDGCLKKSARTRKENKFSAKRLPSTRLGTFLENRVNDFLRRQNHPESGEVT
VRVVHASDKTVEVKPGMKARFVDSGEMAESFPYRTKALFAFEEIDGVDLCFFGMHVQEYGSD
CPPPNQRRVYISYLDSVHFFRPKCLRTAVYHEILIGYLEYVKKLGYTTGHIWACPPSEGDDY
IFHCHPPDQKIPKPKRLQEWYKKMLDKAVSERIVHDYKDIFKQATEDRLTSAKELPYFEGDF
WPNVLEESIKELEQEEEERKREENTSNESTDVTKGDSKNAKKKNNKKTSKNKSSLSRGNKKK
PGMPNVSNDLSQKLYATMEKHKEVFFVIRLIAGPAANSLPPIVDPDPLIPCDLMDGRDAFLT
LARDKHLEFSSLRRAQWSTMCMLVELHTQSQD
Streptococcus parasanguinis dCas9-p300 nucleotides (human codon optimized)
SEQ ID NO: 230
ATGAACGGACTGGTTTTGGGTCTTGCCATCGGGATCGCTAGCGTTGGGGTGGGCATCCTGAA
GAAGGACATAGGAGAAATCATTCATACCAATTCTAGACTGTTTTCAGCTGCCACAGCCGACT
CTAATATTGAACGACGAGGACATCGTGGCGGCAAGAGGCTGACAAGACGAAAAAAACACCGA
AGCATACGACTTCACGATCTTTTCGAGGATTTTGGACTGCTGACGGACTTTTCAAAAGTTTC
CATCAACTTGAATCCGTACCAGTTACGCGTACAGGGTCTGGACAACCAGCTGACAAACGAGG
AGCTGTTTATCGCTCTTAAGAATATCGTGAAGCGCCGGGGGATTAGCTACTTAGACGATGCC
TCTGAAGACGGCGGAACCGTGTCTTCTGATTATGGGAAGGCTGTCGAAGAAAATAGAAAACT
CTTAGCCGAACAGACTCCTGGGCAGATCCAGCTGGACAGATTCGAAAAGTACGGCCAAGTCC
GAGGCGACTTCAATGTCGTGGAGAACGGTGAGAAACGACGTCTGATTAACGTCTTTACAACT
AGCGCCTATTCCAAGGAGGCCGAGAGGATACTGAGGAAGCAGCAGGAGTTCAACAAGAAAAT
AACGGATGAGTTCATCGAGGATTACCTGACCATTCTTACTGGAAAAAGAAAATATTACCATG
GTCCTGGAAACGAAAAGTCCCGGACCGATTACGGGCGGTACACAACCAAAAAGGACCCAGAG
GGCAAATACATCACCCTCGATAATATTTTCGGGATCCTCATCGGTAAATGCACTTTTTACCC
CGATGAGTATCGCGCGTCTAAAGCTTCATATACCGCACAGGAGTTCAATCTGCTCAACGACC
TGAATAACCTGACCGTGCCTACCGAAACCAAAAAACTGTCAGAGGAGCAGAAGAAGACGATA
ATAAAATACGCCAAAACGGCTAAGACCCTTGGCGCTTCTACTCTGCTGAAGTATATAGCCAA
ACTGATCGGTGCTTCCGTTGACCAGATTCACGGGTATAGAATCGACCCAAATAAAAAGCCCG
AAATGCACACCTTCGAGACGTACCGGAAAATGCAATCCCTGGAGACGATCTCAGTGGAGGAA
CTGCCTCGCAAAGTGCTTGACGAACTCGCCCATATTCTGACATTGAACACTGAGCGCGAAGG
CATCGAGGAGGCTATTAATGCCACCTTGAAAGATACGTTTAGTCAGGACCAGGTCCTCGAAC
TCGTGCAGTTCCGCAAAAATAACTCTTCCTTATTCTCAAAGGGATGGCATAGTTTCAGCCTG
AAACTGATGATGGAACTGATTCCTGAACTCTATGAGACTAGTGAAGAGCAGATGACTATACT
GACTCGTCTGGGGAAACAGAAGTCTAAGGAGACAAGTAAACGAACTAAGTACATTGATGAAA
AGGAGCTGACAGAAGAGATTTATAATCCAGTCGTGGCTAAATCCGTCCGTCAGGCTATTAAG
ATCATTAACGAGGCAACGAAAAAGTACGGAATCTTCGATAACATTGTGATCGAAATGGCCCG
TGAGAACAATGAAGAAGATGCTAAGAAGGAGTACATCAAGCGGCAGAAGGCAAACTTGGATG
AGAAGAACGCCGCAATGGAGAAAGCTGCTTTTCAATACAACGGTAAGAAGGAACTCCCGGAT
AACGTCTTCCACGGCCATAAGGAGCTCGCCACAAAAATACGGTTGTGGCACCAGCAGGGGGA
AAAGTGCCTCTACACTGGAAAAAATATCCCTATCTCCGACCTTATTCAAAATCAGTACAAGT
ATGAAATCGACGCCATCCTCCCACTGTCCCTCAGTTTCGACGATAGCCTGTCCAACAAGGTC
CTCGTGCTGGCTACCGCCAATCAGGAAAAGGGCCAAAGAACTCCTTTTCAAGCTCTCGATTC
AATGGATGACGCCTGGAGCTATCGGGAGTTCAAATCCTATGTGAAGGATTCTAAACTCTTGG
GGAATAAAAAGAAGGAATACTTGTTAACAGAGGAGGATATCAGTAAGATCGAGGTGAAACAG
AAGTTTATCGAACGGAATTTAGTTGACACAAGGTACTCAAGTCGCGTGGTTCTTAACGCCCT
TCAGGACTTCTACAAGGAACACCAGTTCGATACCACCATTAGCGTGGTTAGAGGACAATTTA
CATCCCAGCTGCGGCGGAAATGGGGACTGGAGAAGAGCCGAGAAACCTACCATCACCACGCC
GTGGACGCGCTGATTATTGCCGCCAGTAGCCAGCTCCGCCTCTGGAAAAAACAGAACAATCC
TCTTATCAGTTACACCGAAGGCCAGTTTGTGGATCAGGTGACCGGCGAGATTATATCCCTTT
CTGACGACGAATACAAAGAGCTGGTTTTCAAAGCTCCATATGACCACTTTGTTGACACACTG
AAGTCAAAGAAGTTTGAGGATTCTATCCTCTTTTCATACCAGGTGGATTCTAAGTACAACCG
GAAGATTAGCGACGCAACCATATATGCAACTCGTAAAGCCAAATTGGACAAGGAGAATAAAG
AGTATACTTACACTCTGGGTAAAATCAAAGATATCTATGCTCTTGGCACGAAAAGCCCTTCT
AAAACCGGCTTTTACAAGTTTCTGGACCTGTATAATAAAGACAAGTCTCAGTTCCTGATGTT
TCAGAAAGATCGCAAGACCTGGGACGAGGTGATTGAAAAGATCATTGAGCAATACCGGCCTT
TTAAAGAATACGACGAAAACGGAAAAGAAGTAGACTTCAACCCTTTTGAAAAGTACCGGATC
GAAAATGGCCCCATCCGAAAATATTCCAAAAAGGGCAATGGCCCAGAAATAAAGAGTCTCAA
GTATTACGATAACTTACTGGGTAAGTTTGTGGATATCACACCTTCAGAGTCCAAGAACCCCG
TAGCTCTGCTGTCCCTGAACCCGTGGAGGACTGACGTCTACTACAACACCGAAACATCCAAG
TATGAGTTCCTTGGGCTGAAGTATGCTGATTTGTGCTTCGAAAAGGGGGGTGCATATGGTAT
CTCTGAGGTGAAATACAATAAAATAAGAGAGAAAGAAGGTATCGGAAAGGAGTCTGAATTCA
AATTTACCCTTTATAAGAATGACCTGATTCTGATCAAAGATACAGAGACAAACTGCCAACAG
ATTTTTCGCTTCTGGTCTCGAACTGGAAAAGACAATCCTAAATCATTTGAGAAGCATAAAAT
TGAGCTTAAACCGTATGAAAAGGCTCGTTTCGAGAAGGGTGAAGAACTGGAGGTGCTGGGAA
AGGTGCCACCTAGTTCAAACCAACTGCAGAAAAATATGCAAATTGAGAACCTCTCCATCTAC
AAGGTGAAGACTGATGTGCTTGGCAATAAGCATTTTATCAAAAAGGAAGGAGAAGAACCTAA
GCTCAAATTTACCGGTCCTAAGAAAAAGCGGAAAGTGGCTagCattttcaaaccagaagaac
tacgacaggcactgatgccaactttggaggcactttaccgtcaggatccagaatcccttccc
tttcgtcaacctgtggaccctcagcttttaggaatccctgattactttgatattgtgaagag
ccccatggatctttctaccattaagaggaagttagacactggacagtatcaggagccctggc
agtatgtcgatgatatttggcttatgttcaataatgcctggttatataaccggaaaacatca
cgggtatacaaatactgctccaagctctctgaggtctttgaacaagaaattgacccagtgat
gcaaagccttggatactgttgtggcagaaagttggagttctctccacagacactgtgttgct
acggcaaacagttgtgcacaatacctcgtgatgccacttattacagttaccagaacaggtat
catttctgtgagaagtgtttcaatgagatccaaggggagagcgtttctttgggggatgaccc
ttcccagcctcaaactacaataaataaagaacaattttccaagagaaaaaatgacacactgg
atcctgaactgtttgttgaatgtacagagtgcggaagaaagatgcatcagatctgtgtcctt
caccatgagatcatctggcctgctggattcgtctgtgatggctgtttaaagaaaagtgcacg
aactaggaaagaaaataagttttctgctaaaaggttgccatctaccagacttggcacctttc
tagagaatcgtgtgaatgactttctgaggcgacagaatcaccctgagtcaggagaggtcact
gttagagtagttcatgcttctgacaaaaccgtggaagtaaaaccaggcatgaaagcaaggtt
tgtggacagtggagagatggcagaatcctttccataccgaaccaaagccctctttgcctttg
aagaaattgatggtgttgacctgtgcttctttggcatgcatgttcaagagtatggctctgac
tgccctccacccaaccagaggagagtatacatatcttacctcgatagtgttcatttcttccg
tcctaaatgcttgaggactgcagtctatcatgaaatcctaattggatatttagaatatgtca
agaaattaggttacacaacagggcatatttgggcatgtccaccaagtgagggagatgattat
atcttccattgccatcctcctgaccagaagatacccaagcccaagcgactgcaggaatggta
caaaaaaatgcttgacaaggctgtatcagagcgtattgtccatgactacaaggatattttta
aacaagctactgaagatagattaacaagtgcaaaggaattgccttatttcgagggtgatttc
tggcccaatgttctggaagaaagcattaaggaactggaacaggaggaagaagagagaaaacg
agaggaaaacaccagcaatgaaagcacagatgtgaccaagggagacagcaaaaatgctaaaa
agaagaataataagaaaaccagcaaaaataagagcagcctgagtaggggcaacaagaagaaa
cccgggatgcccaatgtatctaacgacctctcacagaaactatatgccaccatggagaagca
taaagaggtcttctttgtgatccgcctcattgctggccctgctgccaactccctgcctccca
ttgttgatcctgatcctctcatcccctgcgatctgatggatggtcgggatgcgtttctcacg
ctggcaagggacaagcacctggagttctcttcactccgaagagcccagtggtccaccatgtg
catgctggtggagctgcacACGCAGAGCCAGGAC
Streptococcus parasanguinis gRNA scaffold-RNA
SEQ ID NO: 231
GUUUUUGUACUCUCAAGAUUUCGAAAAAUCUUGCAGAAGCUACAAAGAUAAGGCUUCAUGCC
GAAUUCAACACCCUGUCAUUUAUGGCGGGGUGUUUUCGUUUU
Streptococcus parasanguinis gRNA scaffold-DNA
SEQ ID NO: 232
GTTTTTGTACTCTCAAGATTTCGAAAAATCTTGCAGAAGCTACAAAGATAAGGCTTCATGCC
GAATTCAACACCCTGTCATTTATGGCGGGGTGTTTTCGTTTT
gRNA scaffold for Streptococcus dysgalactiae Cas9-RNA
SEQ ID NO: 233
GUUUUAGAGCUAUGUCGAAACGUAGCAAGUUAAAAUAAGGCUAGUCCGUUAUCAACUUGAAA
AAGUGGCACCGAGUCGGUGCUUUUUGUAUCUUUAUUUU
gRNA scaffold for Streptococcus dysgalactiae Cas9-DNA
SEQ ID NO: 234
GTTTTAGAGCTATGTCGAAACGTAGCAAGTTAAAATAAGGCTAGTCCGTTATCAACTTGAAA
AAGTGGCACCGAGTCGGTGCTTTTTGTATCTTTATTTT
Streptococcus dysgalactiae Cas9 nuclease, protein
SEQ ID NO: 235
MNKPYSIGLDIGTNSVGWSIITDDYKVPAKKMRVLGNTDKEYIKKNLIGALLFDGGNTAADR
RLKRTARRRYTRRRNRILYLQEIFAEEMSKVDDSFFHRLEDSFLVEEDKRGSKYPIFATMQE
EKYYHEKFPTIYHLRKELADKKEKADLRLVYLALAHIIKFRGHFLIEDDREDVRNTDIQKQY
QAFLEIFDTTFENNHLLSQNVDVEAILTDKISKSAKKDRILAQYPNQKSTGIFAEFLKLIVG
NQADFKKHENLEDKTPLQFAKDSYDEDLENLLGQIGDEFADLESVAKKLYDSVLLSGILTVT
DLSTKAPLSASMIQRYDEHHEDLKHLKQFVKASLPENYREVFADSSKDGYAGYIEGKTNQEA
FYKYLLKLLTKQEGSEYFLEKIKNEDFLRKQRTFDNGSIPHQVHLTELRAIIRRQSEYYPFL
KENQDRIEKILTFRIPYYVGPLAREKSDFAWMTRKTDDSIRPWNFEDLVDKEKSAEAFIHRM
TNNDLYLPEEKVLPKHSLIYEKFTVYNELTKVRFLAEGFKDFQFLNRKQKETIFNSLFKEKR
KVTEKDIISFLNKVDGYEGIAIKGIEKQFNASLSTYHDLKKILGKDFLDNTDNELILEDIVQ
TLTLFEDREMIKKCLDIYKDFFTESQLKKLYRRHYTGWGRLSAKLINGIRNKENQKTILDYL
IDDGSANRNFMQLINDDDLSFKPIIDKARTGSHSDNLKEVVGELAGSPAIKKGILQSLKIVD
ELVKVMGYEPEQIVVEMARENQTTAKGLSRSRQRLTTLRESLANLKSNILEEKKPKYVKDQV
ENHHLSDDRLFLYYLQNGRDMYTKKALDIDNLSQYDIDHIIPQAFIKDDSIDNRVLVSSAKN
RGKSDDVPSIEIVKARKMFWKNLLDAKLMSQRKYDNLTKAERGGLTSDDKARFIQRQLVETR
QITKHVARILDERENNEVDNGKKICKVKIVTLKSNLVSNFRKEFGFYKIREVNDYHHAHDAY
LNAVVAKAILTKYPQLEPEFVYGMYRQKKLSKIVHEDKEEKYSEATRKMFFYSNLMNMFKRV
VRLADGSIVVRPVIETGRYMGKTAWDKKKHFATVRKVLSYPQNNIVKKTEIQTGGFSKESIL
AHGNSDKLIPRKTKDIYLDPKKYGGFDSPIVAYSVLVVADIKKGKAQKLKTVTELLGITIME
RSRFEKNPSAFLESKGYLNIRDDKLMILPKYSLFELENGRRRLLASAGELQKGNELALPTQF
MKFLYLASRYNESKGKPEEIEKKQEFVNQHVSYEDDILQLINDESKRVILADANLEKINKLY
QDNKENIPVDELANNIINLFTFTSLGAPAAFKFEDKIVDRKRYTSTKEVLNSTLIHQSITGL
YETRIDLGKLGED
Streptococcus dysgalactiae Cas9 nuclease, DNA
SEQ ID NO: 236
ATGAACAAGCCTTATTCAATAGGATTAGATATAGGGACAAATTCTGTGGGGTGGAGTATAAT
CACCGACGATTACAAGGTGCCTGCAAAGAAGATGCGCGTGCTCGGCAATACAGACAAGGAAT
ATATTAAGAAGAACCTGATCGGGGCCCTCCTTTTTGACGGTGGCAACACAGCAGCTGACCGC
CGCCTCAAGAGGACCGCTCGGAGACGGTATACTCGCCGGCGTAATCGGATCCTGTATTTGCA
GGAAATTTTTGCTGAAGAAATGTCTAAGGTGGATGATTCATTCTTTCACCGGCTCGAAGACT
CCTTTCTGGTGGAGGAAGACAAGAGGGGCTCAAAGTACCCAATCTTCGCCACAATGCAAGAA
GAGAAATACTACCACGAGAAGTTTCCCACAATCTATCATCTCAGGAAAGAGCTGGCCGATAA
AAAAGAGAAGGCCGATTTGCGACTGGTTTACTTGGCCTTGGCACACATCATAAAGTTCCGGG
GACACTTTCTGATTGAAGACGACCGTTTTGACGTCCGCAACACTGATATACAGAAGCAATAC
CAAGCGTTCCTTGAGATCTTTGACACCACATTTGAAAACAACCATCTGCTGAGCCAAAATGT
GGACGTGGAAGCCATTCTGACTGATAAGATCTCTAAATCTGCCAAAAAGGACAGAATCCTTG
CCCAGTACCCCAACCAGAAGTCAACTGGCATTTTCGCCGAGTTTCTGAAGTTGATAGTTGGC
AATCAGGCCGATTTTAAGAAGCACTTCAATTTGGAGGACAAAACGCCTCTCCAATTCGCCAA
GGACTCATATGATGAGGACCTGGAGAATCTGCTTGGCCAAATCGGGGATGAGTTCGCTGATC
TTTTTAGCGTGGCAAAGAAGCTCTATGACTCTGTACTCCTGAGCGGAATCCTGACAGTTACC
GATCTTTCAACAAAGGCACCCCTGAGTGCAAGCATGATTCAACGCTACGACGAGCACCATGA
GGATCTGAAACATCTGAAGCAGTTCGTCAAGGCTTCTCTGCCTGAAAACTATCGGGAGGTCT
TCGCCGACTCATCTAAGGACGGCTACGCCGGATACATCGAGGGAAAGACAAATCAGGAGGCT
TTCTACAAGTACCTGTTGAAGCTGCTTACAAAACAGGAGGGGAGCGAATACTTCCTGGAGAA
GATCAAAAACGAGGACTTCCTGCGTAAACAGAGGACTTTCGATAATGGCTCCATTCCTCACC
AGGTGCATCTCACGGAACTGAGAGCTATCATTAGACGTCAGAGTGAGTATTACCCATTTCTG
AAGGAGAACCAAGACCGAATCGAAAAAATTCTGACGTTCCGGATCCCTTACTATGTCGGACC
TTTAGCTAGGGAAAAAAGTGACTTCGCCTGGATGACCCGAAAGACAGATGATAGTATCAGAC
CATGGAACTTTGAAGACCTGGTGGACAAAGAGAAGAGCGCCGAGGCTTTTATTCACAGGATG
ACCAATAATGATCTCTATCTGCCTGAAGAGAAGGTGCTGCCCAAACACAGTCTCATCTACGA
AAAATTTACAGTCTATAACGAACTGACAAAGGTCCGCTTTCTGGCTGAAGGATTCAAGGACT
TTCAATTTCTGAACCGGAAGCAGAAGGAAACTATCTTTAACTCATTGTTTAAGGAAAAGAGG
AAGGTTACCGAAAAAGACATCATCTCCTTTTTAAACAAGGTAGATGGGTACGAAGGGATTGC
CATTAAAGGCATTGAGAAACAGTTTAACGCCAGCCTTTCAACCTACCATGATCTCAAGAAGA
TCCTCGGAAAAGATTTCCTTGACAATACCGACAACGAACTTATCCTGGAGGATATAGTGCAG
ACACTCACTCTGTTCGAGGACAGGGAAATGATAAAGAAGTGCCTCGACATATATAAAGACTT
CTTTACCGAGAGTCAACTGAAAAAGTTGTATAGAAGGCATTACACCGGTTGGGGCCGACTGA
GTGCAAAACTCATTAACGGCATCCGGAATAAGGAGAATCAAAAGACTATCCTCGATTACCTC
ATCGATGACGGAAGCGCAAACAGAAACTTCATGCAACTCATCAACGATGATGACCTGTCTTT
CAAACCAATTATAGACAAAGCCAGGACTGGGAGCCATAGTGACAATCTGAAGGAAGTGGTGG
GAGAGCTGGCAGGCAGCCCCGCAATTAAGAAGGGGATCCTGCAGAGCCTCAAAATTGTCGAT
GAACTCGTGAAGGTCATGGGCTATGAACCTGAACAGATTGTTGTAGAGATGGCCCGAGAGAA
CCAGACTACTGCGAAGGGACTTAGCCGGAGCAGACAACGACTGACCACTTTGCGAGAGAGTC
TGGCGAACCTGAAGTCTAATATTCTCGAGGAAAAAAAGCCAAAGTACGTGAAGGACCAGGTG
GAGAATCACCACCTGAGCGACGACAGACTCTTTCTGTATTATCTGCAGAACGGCAGAGATAT
GTATACGAAGAAGGCACTGGACATAGACAACCTGAGTCAGTATGACATCGATcaCATTATCC
CTCAGGCCTTCATCAAAGACGATTCAATCGACAATCGCGTACTTGTTAGCAGTGCGAAAAAC
CGGGGAAAGTCTGATGACGTCCCATCCATCGAAATAGTGAAGGCAAGGAAGATGTTCTGGAA
GAATCTGCTGGATGCCAAATTAATGTCACAACGGAAGTACGACAACCTGACAAAGGCAGAAA
GGGGGGGCTTAACAAGCGACGATAAGGCAAGGTTTATCCAGAGGCAGTTGGTCGAGACCAGG
CAAATCACCAAACACGTCGCCCGGATCCTGGATGAACGCTTCAACAATGAAGTCGACAATGG
CAAAAAAATCTGTAAAGTCAAGATAGTGACACTGAAGTCAAATCTGGTGAGCAACTTCCGGA
AAGAATTCGGCTTCTATAAAATTCGCGAAGTGAACGACTATCACCATGCGCACGACGCTTAC
CTGAATGCAGTCGTGGCGAAAGCCATTTTGACCAAGTACCCCCAGCTGGAGCCTGAGTTTGT
GTACGGAATGTACCGACAAAAGAAGCTGAGCAAGATTGTACACGAGGATAAGGAAGAGAAAT
ACTCCGAGGCCACTCGGAAGATGTTCTTCTACTCTAATCTGATGAACATGTTTAAGAGAGTG
GTGAGGTTGGCAGACGGCTCCATTGTTGTAAGGCCAGTGATCGAGACTGGGCGATACATGGG
CAAGACAGCGTGGGACAAGAAGAAGCATTTCGCAACCGTACGGAAAGTCCTGTCCTACCCGC
AGAATAACATTGTGAAGAAGACAGAAATACAAACCGGTGGTTTCTCAAAAGAGTCCATTTTA
GCCCATGGCAACAGTGACAAATTGATTCCACGGAAGACCAAAGATATTTATCTGGACCCTAA
AAAATACGGCGGATTCGACTCACCGATCGTGGCATACAGCGTATTGGTGGTGGCCGATATTA
AGAAGGGTAAAGCCCAGAAACTCAAGACTGTTACCGAGCTCCTGGGTATCACTATAATGGAG
AGAAGCCGGTTTGAGAAGAACCCTAGCGCCTTTTTGGAATCCAAGGGGTATCTGAACATTCG
GGACGATAAGCTGATGATCTTGCCTAAATACAGCCTTTTTGAACTGGAGAATGGACGAAGGC
GCCTGCTTGCCTCAGCGGGGGAACTGCAGAAAGGCAATGAGCTGGCCCTTCCTACCCAGTTC
ATGAAATTTTTGTATCTGGCTAGTAGGTATAACGAGTCAAAAGGCAAGCCAGAGGAGATCGA
AAAGAAGCAGGAATTTGTAAACCAGCATGTGTCATACTTTGATGATATCCTGCAGTTAATCA
ATGACTTCAGTAAACGAGTCATTCTCGCAGACGCCAACTTGGAGAAAATTAATAAGCTGTAC
CAGGACAACAAAGAGAATATACCAGTCGACGAGCTTGCAAATAACATTATTAACCTGTTCAC
TTTTACATCCCTGGGGGCCCCTGCTGCGTTCAAATTTTTCGACAAAATCGTGGATCGAAAGC
GATATACATCCACTAAGGAAGTTCTGAACAGCACTCTCATCCACCAGTCTATCACTGGCCTT
TACGAAACGCGTATTGACTTGGGGAAACTCGGAGAGGAC
Streptococcus dysgalactiae dCas9, protein
(with D10A and H839A underlined)
SEQ ID NO: 237
MDKKYSIGLAIGTNSVGWAVITDDYKVPSKKFKVLGNTDRHSIKKNLIGALLFDSGETAEAT
RLKRTARRRYTRRKNRIRYLQEIFSSEMSKVDDSFFHRLEESFLVEEDKKHERHPIFGNIVD
EVAYHEKYPTIYHLRKKLADSTDKADLRLIYLALAHMIKFRGHFLIEGDLNPDNSDMDKLFI
QLVQTYNQLFEENPINASRVDAKAILSARLSKSRRLENLIAQLPGEKRNGLFGNLIALSLGL
TPNFKSNFDLAEDAKLQLSKDTYDDDLDNLLAQIGDQYADLFLAAKNLSDAILLSDILRVNS
EITKAPLSASMIKRYDEHHQDLTLLKALVRQQLPEKYKEIFFDQSKNGYAGYIDGGASQEEF
YKFIKPILEKMDGTEELLAKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPEFLSGKQKEAIVDLLEKTNRK
VTVKQLKEDYFKKIECFDSVEISGVEDRFNASLGTYHDLLKIIKDKDELDNEENEDILEDIV
LTLTLFEDKEMIEERLKKYANLFDDKVMKQLKRRHYTGWGRLSRKLINGIRDKQSGKTILDE
LKSDGFANRNFMQLINDDSLTFKEAIQKAQVSGQGHSLHEQIANLAGSPAIKKGILQSVKVV
DELVKVMGHKPENIVIEMARENQTTQKGQKNSRERMKRIEEGIKELGSQILKEHPVENTQLQ
NEKLYLYYLQNGRDMYVDQELDINRLSDYDVDAIVPQSFIKDDSIDNKILTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYTKLESEFVYGDYKVYDVRKMIAKSEQEIGKATAKRFFYSNIMNFFKTEITLANG
EIRKRPLIETNEETGEIVWDKGRDFATVRKVLSMPQVNIVKKTEVQTGALTNESIYARGSED
KLISRKHRFESSKYGGFGSPTVTYSVLVVAKSKVQDGKVKKIKTGKELIGMTLLDKLVFEKN
PLKFIEDKGYGNVQIDKCIKLPKYSLFEFENGTRRMLASVMANNNSRGDLQKANEMFLPAKL
VTLLYHAHKIESSKELEHEAYILDHYNDLYQLLSYIERFASLYVDVEKNISKVKELFSNIES
YSISEICSSVINLLTLTASGAPADEKFLGTTIPRKRYGSPQSILSSTLIHQSITGLYETRID
LSQLGGD
Streptococcus dysgalactiae dCas9, DNA
SEQ ID NO: 238
ATGGATAAGAAGTACTCCATTGGACTGGCAATTGGGACAAATTCAGTGGGATGGGCTGTGAT
AACGGATGATTATAAAGTGCCATCTAAGAAGTTTAAAGTGCTGGGGAACACAGACAGACACT
CAATCAAAAAGAATTTGATTGGGGCCCTCCTCTTCGACTCAGGTGAGACCGCTGAAGCTACT
CGCCTCAAGAGAACAGCGAGACGGCGGTATACTCGTAGAAAGAACCGCATTCGCTACCTGCA
AGAGATATTTAGCAGCGAAATGAGTAAGGTGGACGATAGCTTCTTCCACAGACTGGAGGAGA
GCTTTCTTGTGGAGGAGGACAAGAAACACGAGCGCCATCCCATCTTTGGTAATATTGTGGAC
GAGGTGGCCTATCATGAGAAGTATCCAACAATTTACCACCTTAGAAAGAAGTTGGCAGATTC
CACCGACAAAGCTGACCTCCGGCTGATCTACCTTGCTCTCGCACATATGATTAAATTCCGGG
GACACTTCTTGATTGAAGGCGACCTTAACCCCGACAACTCAGATATGGACAAGCTCTTCATC
CAGCTCGTACAAACCTACAATCAGCTTTTCGAGGAAAACCCAATTAACGCTTCCAGGGTCGA
CGCAAAAGCGATACTGTCTGCTCGTCTTAGTAAGTCCCGGCGGCTCGAGAACTTAATTGCAC
AGTTGCCCGGCGAAAAGCGTAATGGACTGTTTGGGAATCTCATTGCCCTTTCCCTTGGACTG
ACTCCAAATTTCAAGTCAAATTTCGATCTCGCTGAGGACGCAAAACTGCAGCTGTCTAAGGA
CACTTACGACGATGACCTGGACAACCTGCTGGCTCAGATTGGCGACCAGTACGCCGATTTAT
TCCTCGCCGCAAAAAACCTTTCTGATGCCATCCTGCTGAGCGATATTCTTAGAGTTAACAGT
GAGATTACAAAAGCCCCCCTGAGTGCCTCCATGATTAAGCGCTATGACGAACACCACCAAGA
CTTGACTCTCCTGAAAGCTTTAGTACGGCAACAGCTCCCCGAGAAATATAAGGAGATCTTTT
TCGATCAATCCAAGAACGGATACGCGGGATATATAGATGGAGGGGCTAGCCAAGAGGAATTT
TACAAGTTCATCAAACCAATTTTAGAAAAGATGGACGGAACAGAAGAATTATTGGCCAAGCT
GAATCGGGAGGATCTGCTGAGAAAGCAGAGAACATTCGATAACGGCTCCATACCCCACCAGA
TCCACCTCGGAGAATTACACGCAATTCTTAGACGCCAGGAGGATTTCTACCCCTTCCTGAAA
GACAATCGAGAGAAGATTGAAAAAATACTGACATTTCGGATCCCCTATTACGTGGGTCCTCT
GGCCCGAGGGAATAGTCGGTTCGCCTGGATGACACGTAAGTCAGAAGAGACGATTACCCCCT
GGAATTTTGAGGAAGTGGTTGATAAAGGCGCCAGCGCTCAGTCTTTCATCGAGCGTATGACT
AATTTTGACAAAAACTTGCCCAACGAGAAAGTCCTCCCCAAACACTCCTTACTTTATGAGTA
CTTCACCGTCTATAACGAGCTTACAAAAGTTAAGTACGTAACTGAGGGTATGAGGAAACCAG
AGTTCCTCAGCGGGAAACAAAAGGAGGCCATTGTGGATCTGCTTTTCAAAACAAACAGGAAG
GTTACCGTGAAACAATTAAAGGAGGATTACTTTAAGAAAATCGAGTGCTTCGATAGCGTCGA
GATATCTGGAGTAGAAGACAGGTTCAACGCGTCCCTGGGTACCTACCACGATCTGCTGAAAA
TAATCAAGGACAAGGACTTCCTCGATAATGAGGAAAATGAAGATATCCTGGAGGACATCGTG
CTTACTCTGACACTGTTTGAAGACAAAGAGATGATAGAGGAGAGGCTGAAGAAATATGCAAA
TCTTTTCGATGATAAAGTTATGAAACAGCTTAAGCGAAGGCATTACACCGGGTGGGGGAGGC
TGAGCCGGAAGCTTATCAATGGGATCAGGGACAAGCAGAGCGGGAAGACTATATTGGATTTT
CTGAAGTCTGATGGGTTTGCAAATAGGAACTTCATGCAGCTCATTAATGACGATTCACTGAC
ATTTAAGGAGGCTATTCAGAAGGCTCAAGTAAGTGGACAGGGGCATAGCCTGCACGAACAGA
TTGCTAATCTCGCCGGATCTCCAGCAATTAAGAAGGGCATCCTGCAGAGTGTTAAAGTTGTG
GACGAGCTGGTCAAGGTGATGGGCCACAAGCCTGAAAATATAGTTATTGAGATGGCGAGGGA
AAACCAAACAACTCAGAAAGGACAAAAAAACTCCCGCGAACGAATGAAAAGGATCGAAGAGG
GCATTAAAGAATTGGGCTCCCAGATTCTCAAAGAACATCCTGTTGAAAATACCCAGCTGCAG
AACGAGAAGCTGTATCTGTATTATCTGCAGAACGGGAGAGATATGTACGTCGACCAGGAGCT
GGACATTAACCGATTGTCTGACTACGATGTCGACGCAATCGTTCCGCAAAGCTTCATAAAGG
ATGATTCCATCGACAATAAAATTCTCACTCGGAGCGACAAAAATCGAGGAAAGTCTGACAAT
GTGCCCAGCGAAGAGGTGGTAAAGAAGATGAAGAACTACTGGAGACAGCTTCTGAATGCTAA
ACTGATTACTCAACGTAAGTTCGACAATCTGACAAAGGCTGAAAGGGGGGGTCTGAGCGAGC
TGGATAAGGCTGGGTTCATTAAAAGGCAGTTGGTCGAAACCCGACAAATCACCAAGCATGTT
GCTCAGATCTTGGACTCAAGAATGAACACAAAATATGATGAAAACGATAAACTGATTAGGGA
GGTGAAGGTGATCACTCTTAAGAGCAAGTTAGTCTCAGACTTCAGGAAAGATTTTCAGTTCT
ATAAGGTGCGGGAGATTAACAACTATCATCATGCCCACGACGCGTATCTCAACGCGGTTGTG
GGAACCGCCCTGATCAAAAAGTACACTAAGCTGGAGAGCGAGTTTGTTTATGGAGATTATAA
AGTGTACGACGTAAGGAAGATGATCGCGAAGTCAGAGCAGGAGATCGGTAAAGCTACCGCAA
AGCGCTTCTTCTACAGTAACATTATGAACTTCTTCAAGACAGAGATTACGCTCGCCAATGGC
GAGATACGGAAGAGACCCCTGATTGAGACTAACGAAGAAACAGGCGAGATCGTTTGGGACAA
AGGAAGAGATTTCGCTACAGTGCGGAAAGTGCTCTCTATGCCCCAGGTGAATATCGTCAAGA
AGACAGAAGTGCAGACCGGAGCGTTAACCAACGAGAGCATATATGCACGCGGCTCCTTTGAT
AAGCTGATCTCCAGGAAGCACAGGTTCGAGTCCTCCAAGTACGGGGGCTTCGGCAGCCCAAC
TGTTACTTACTCCGTCCTGGTGGTGGCCAAAAGCAAAGTCCAAGACGGGAAGGTCAAAAAGA
TCAAGACAGGGAAAGAGCTGATTGGCATGACACTGTTGGACAAGTTGGTGTTCGAGAAAAAC
CCCCTGAAATTTATAGAAGACAAGGGGTACGGAAACGTGCAGATCGATAAGTGCATTAAGCT
GCCTAAGTACTCTTTATTCGAGTTCGAAAACGGCACCCGTCGGATGTTAGCCTCCGTCATGG
CGAATAATAACAGCAGGGGCGACTTGCAGAAAGCTAACGAAATGTTTCTGCCTGCCAAGTTG
GTGACATTGCTGTATCACGCCCACAAGATTGAATCAAGCAAAGAGCTGGAGCACGAGGCATA
CATCCTTGATCATTACAATGATTTGTATCAGCTCCTGTCTTACATCGAACGGTTCGCCAGCC
TGTATGTGGACGTAGAGAAGAACATATCTAAGGTAAAGGAGTTGTTTTCCAACATCGAATCC
TACAGCATCAGTGAGATCTGCTCCTCTGTGATTAATCTCTTAACTTTAACAGCTAGCGGGGC
CCCGGCCGACTTTAAATTCTTAGGTACAACGATCCCGCGCAAGAGGTACGGCTCCCCCCAAT
CAATTCTCTCCAGCACACTGATTCACCAGAGCATCACCGGCTTATATGAAACGAGGATTGAC
CTGAGTCAGCTTGGTGGCGAC
Streptococcus dysgalactiae dCas9-KRAB, protein
SEQ ID NO: 239
MDKKYSIGLAIGTNSVGWAVITDDYKVPSKKFKVLGNTDRHSIKKNLIGALLFDSGETAEAT
RLKRTARRRYTRRKNRIRYLQEIFSSEMSKVDDSFFHRLEESFLVEEDKKHERHPIFGNIVD
EVAYHEKYPTIYHLRKKLADSTDKADLRLIYLALAHMIKFRGHFLIEGDLNPDNSDMDKLFI
QLVQTYNQLFEENPINASRVDAKAILSARLSKSRRLENLIAQLPGEKRNGLFGNLIALSLGL
TPNFKSNFDLAEDAKLQLSKDTYDDDLDNLLAQIGDQYADLFLAAKNLSDAILLSDILRVNS
EITKAPLSASMIKRYDEHHQDLTLLKALVRQQLPEKYKEIFFDQSKNGYAGYIDGGASQEEF
YKFIKPILEKMDGTEELLAKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPEFLSGKQKEAIVDLLEKTNRK
VTVKQLKEDYFKKIECFDSVEISGVEDRFNASLGTYHDLLKIIKDKDFLDNEENEDILEDIV
LTLTLFEDKEMIEERLKKYANLFDDKVMKQLKRRHYTGWGRLSRKLINGIRDKQSGKTILDF
LKSDGFANRNEMQLINDDSLTFKEAIQKAQVSGQGHSLHEQIANLAGSPAIKKGILQSVKVV
DELVKVMGHKPENIVIEMARENQTTQKGQKNSRERMKRIEEGIKELGSQILKEHPVENTQLQ
NEKLYLYYLQNGRDMYVDQELDINRLSDYDVDAIVPQSFIKDDSIDNKILTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYTKLESEFVYGDYKVYDVRKMIAKSEQEIGKATAKRFFYSNIMNFFKTEITLANG
EIRKRPLIETNEETGEIVWDKGRDFATVRKVLSMPQVNIVKKTEVQTGALTNESIYARGSED
KLISRKHRFESSKYGGFGSPTVTYSVLVVAKSKVQDGKVKKIKTGKELIGMTLLDKLVFEKN
PLKFIEDKGYGNVQIDKCIKLPKYSLFEFENGTRRMLASVMANNNSRGDLQKANEMFLPAKL
VTLLYHAHKIESSKELEHEAYILDHYNDLYQLLSYIERFASLYVDVEKNISKVKELFSNIES
YSISEICSSVINLLTLTASGAPADFKFLGTTIPRKRYGSPQSILSSTLIHQSITGLYETRID
LSQLGGDTGPKKKRKVASMDAKSLTAWSRTLVTFKDVFVDFTREEWKLLDTAQQIVYRNVML
ENYKNLVSLGYQLTKPDVILRLEKGEEP
Streptococcus dysgalactiae dCas9-KRAB, DNA
SEQ ID NO: 240
ATGGATAAGAAGTACTCCATTGGACTGGCAATTGGGACAAATTCAGTGGGATGGGCTGTGAT
AACGGATGATTATAAAGTGCCATCTAAGAAGTTTAAAGTGCTGGGGAACACAGACAGACACT
CAATCAAAAAGAATTTGATTGGGGCCCTCCTCTTCGACTCAGGTGAGACCGCTGAAGCTACT
CGCCTCAAGAGAACAGCGAGACGGCGGTATACTCGTAGAAAGAACCGCATTCGCTACCTGCA
AGAGATATTTAGCAGCGAAATGAGTAAGGTGGACGATAGCTTCTTCCACAGACTGGAGGAGA
GCTTTCTTGTGGAGGAGGACAAGAAACACGAGCGCCATCCCATCTTTGGTAATATTGTGGAC
GAGGTGGCCTATCATGAGAAGTATCCAACAATTTACCACCTTAGAAAGAAGTTGGCAGATTC
CACCGACAAAGCTGACCTCCGGCTGATCTACCTTGCTCTCGCACATATGATTAAATTCCGGG
GACACTTCTTGATTGAAGGCGACCTTAACCCCGACAACTCAGATATGGACAAGCTCTTCATC
CAGCTCGTACAAACCTACAATCAGCTTTTCGAGGAAAACCCAATTAACGCTTCCAGGGTCGA
CGCAAAAGCGATACTGTCTGCTCGTCTTAGTAAGTCCCGGCGGCTCGAGAACTTAATTGCAC
AGTTGCCCGGCGAAAAGCGTAATGGACTGTTTGGGAATCTCATTGCCCTTTCCCTTGGACTG
ACTCCAAATTTCAAGTCAAATTTCGATCTCGCTGAGGACGCAAAACTGCAGCTGTCTAAGGA
CACTTACGACGATGACCTGGACAACCTGCTGGCTCAGATTGGCGACCAGTACGCCGATTTAT
TCCTCGCCGCAAAAAACCTTTCTGATGCCATCCTGCTGAGCGATATTCTTAGAGTTAACAGT
GAGATTACAAAAGCCCCCCTGAGTGCCTCCATGATTAAGCGCTATGACGAACACCACCAAGA
CTTGACTCTCCTGAAAGCTTTAGTACGGCAACAGCTCCCCGAGAAATATAAGGAGATCTTTT
TCGATCAATCCAAGAACGGATACGCGGGATATATAGATGGAGGGGCTAGCCAAGAGGAATTT
TACAAGTTCATCAAACCAATTTTAGAAAAGATGGACGGAACAGAAGAATTATTGGCCAAGCT
GAATCGGGAGGATCTGCTGAGAAAGCAGAGAACATTCGATAACGGCTCCATACCCCACCAGA
TCCACCTCGGAGAATTACACGCAATTCTTAGACGCCAGGAGGATTTCTACCCCTTCCTGAAA
GACAATCGAGAGAAGATTGAAAAAATACTGACATTTCGGATCCCCTATTACGTGGGTCCTCT
GGCCCGAGGGAATAGTCGGTTCGCCTGGATGACACGTAAGTCAGAAGAGACGATTACCCCCT
GGAATTTTGAGGAAGTGGTTGATAAAGGCGCCAGCGCTCAGTCTTTCATCGAGCGTATGACT
AATTTTGACAAAAACTTGCCCAACGAGAAAGTCCTCCCCAAACACTCCTTACTTTATGAGTA
CTTCACCGTCTATAACGAGCTTACAAAAGTTAAGTACGTAACTGAGGGTATGAGGAAACCAG
AGTTCCTCAGCGGGAAACAAAAGGAGGCCATTGTGGATCTGCTTTTCAAAACAAACAGGAAG
GTTACCGTGAAACAATTAAAGGAGGATTACTTTAAGAAAATCGAGTGCTTCGATAGCGTCGA
GATATCTGGAGTAGAAGACAGGTTCAACGCGTCCCTGGGTACCTACCACGATCTGCTGAAAA
TAATCAAGGACAAGGACTTCCTCGATAATGAGGAAAATGAAGATATCCTGGAGGACATCGTG
CTTACTCTGACACTGTTTGAAGACAAAGAGATGATAGAGGAGAGGCTGAAGAAATATGCAAA
TCTTTTCGATGATAAAGTTATGAAACAGCTTAAGCGAAGGCATTACACCGGGTGGGGGAGGC
TGAGCCGGAAGCTTATCAATGGGATCAGGGACAAGCAGAGCGGGAAGACTATATTGGATTTT
CTGAAGTCTGATGGGTTTGCAAATAGGAACTTCATGCAGCTCATTAATGACGATTCACTGAC
ATTTAAGGAGGCTATTCAGAAGGCTCAAGTAAGTGGACAGGGGCATAGCCTGCACGAACAGA
TTGCTAATCTCGCCGGATCTCCAGCAATTAAGAAGGGCATCCTGCAGAGTGTTAAAGTTGTG
GACGAGCTGGTCAAGGTGATGGGCCACAAGCCTGAAAATATAGTTATTGAGATGGCGAGGGA
AAACCAAACAACTCAGAAAGGACAAAAAAACTCCCGCGAACGAATGAAAAGGATCGAAGAGG
GCATTAAAGAATTGGGCTCCCAGATTCTCAAAGAACATCCTGTTGAAAATACCCAGCTGCAG
AACGAGAAGCTGTATCTGTATTATCTGCAGAACGGGAGAGATATGTACGTCGACCAGGAGCT
GGACATTAACCGATTGTCTGACTACGATGTCGACGCAATCGTTCCGCAAAGCTTCATAAAGG
ATGATTCCATCGACAATAAAATTCTCACTCGGAGCGACAAAAATCGAGGAAAGTCTGACAAT
GTGCCCAGCGAAGAGGTGGTAAAGAAGATGAAGAACTACTGGAGACAGCTTCTGAATGCTAA
ACTGATTACTCAACGTAAGTTCGACAATCTGACAAAGGCTGAAAGGGGGGGTCTGAGCGAGC
TGGATAAGGCTGGGTTCATTAAAAGGCAGTTGGTCGAAACCCGACAAATCACCAAGCATGTT
GCTCAGATCTTGGACTCAAGAATGAACACAAAATATGATGAAAACGATAAACTGATTAGGGA
GGTGAAGGTGATCACTCTTAAGAGCAAGTTAGTCTCAGACTTCAGGAAAGATTTTCAGTTCT
ATAAGGTGCGGGAGATTAACAACTATCATCATGCCCACGACGCGTATCTCAACGCGGTTGTG
GGAACCGCCCTGATCAAAAAGTACACTAAGCTGGAGAGCGAGTTTGTTTATGGAGATTATAA
AGTGTACGACGTAAGGAAGATGATCGCGAAGTCAGAGCAGGAGATCGGTAAAGCTACCGCAA
AGCGCTTCTTCTACAGTAACATTATGAACTTCTTCAAGACAGAGATTACGCTCGCCAATGGC
GAGATACGGAAGAGACCCCTGATTGAGACTAACGAAGAAACAGGCGAGATCGTTTGGGACAA
AGGAAGAGATTTCGCTACAGTGCGGAAAGTGCTCTCTATGCCCCAGGTGAATATCGTCAAGA
AGACAGAAGTGCAGACCGGAGCGTTAACCAACGAGAGCATATATGCACGCGGCTCCTTTGAT
AAGCTGATCTCCAGGAAGCACAGGTTCGAGTCCTCCAAGTACGGGGGCTTCGGCAGCCCAAC
TGTTACTTACTCCGTCCTGGTGGTGGCCAAAAGCAAAGTCCAAGACGGGAAGGTCAAAAAGA
TCAAGACAGGGAAAGAGCTGATTGGCATGACACTGTTGGACAAGTTGGTGTTCGAGAAAAAC
CCCCTGAAATTTATAGAAGACAAGGGGTACGGAAACGTGCAGATCGATAAGTGCATTAAGCT
GCCTAAGTACTCTTTATTCGAGTTCGAAAACGGCACCCGTCGGATGTTAGCCTCCGTCATGG
CGAATAATAACAGCAGGGGCGACTTGCAGAAAGCTAACGAAATGTTTCTGCCTGCCAAGTTG
GTGACATTGCTGTATCACGCCCACAAGATTGAATCAAGCAAAGAGCTGGAGCACGAGGCATA
CATCCTTGATCATTACAATGATTTGTATCAGCTCCTGTCTTACATCGAACGGTTCGCCAGCC
TGTATGTGGACGTAGAGAAGAACATATCTAAGGTAAAGGAGTTGTTTTCCAACATCGAATCC
TACAGCATCAGTGAGATCTGCTCCTCTGTGATTAATCTCTTAACTTTAACAGCTAGCGGGGC
CCCGGCCGACTTTAAATTCTTAGGTACAACGATCCCGCGCAAGAGGTACGGCTCCCCCCAAT
CAATTCTCTCCAGCACACTGATTCACCAGAGCATCACCGGCTTATATGAAACGAGGATTGAC
CTGAGTCAGCTTGGTGGCGACACCGGTCCTAAGAAAAAGCGGAAAGTGGctagCatggatgc
taagtcactaactgcctggtccCGGACACTGGTGACCTTCAAGGATGTATTTGTGGACTTCA
CCAGGGAGGAGTGGAAGCTGCTGGACACTGCTCAGCAGATCGTGTACAGAAATGTGATGCTG
GAGAACTATAAGAACCTGGTTTCCTTGGGTTATCAGCTTACTAAGCCAGATGTGATCCTCCG
GTTGGAGAAGGGAGAAGAGCCC
Streptococcus agalactiae Cas9 nuclease, protein
SEQ ID NO: 241
MNKPYSIGLDIGTNSVGWSIITDDYKVPAKKMRVLGNTDKEYIKKNLIGALLEDGGNTAADR
RLKRTARRRYTRRRNRILYLQEIFAEEMSKVDDSFFHRLEDSFLVEEDKRGSKYPIFATMQE
EKYYHEKFPTIYHLRKELADKKEKADLRLVYLALAHIIKFRGHFLIEDDREDVRNTDIQKQY
QAFLEIFDTTFENNHLLSQNVDVEAILTDKISKSAKKDRILAQYPNQKSTGIFAEFLKLIVG
NQADFKKHFNLEDKTPLQFAKDSYDEDLENLLGQIGDEFADLESVAKKLYDSVLLSGILTVT
DLSTKAPLSASMIQRYDEHHEDLKHLKQFVKASLPENYREVFADSSKDGYAGYIEGKTNQEA
FYKYLLKLLTKQEGSEYFLEKIKNEDFLRKQRTFDNGSIPHQVHLTELRAIIRRQSEYYPFL
KENQDRIEKILTFRIPYYVGPLAREKSDFAWMTRKTDDSIRPWNFEDLVDKEKSAEAFIHRM
TNNDLYLPEEKVLPKHSLIYEKFTVYNELTKVRFLAEGFKDFQFLNRKQKETIENSLFKEKR
KVTEKDIISFLNKVDGYEGIAIKGIEKQFNASLSTYHDLKKILGKDELDNTDNELILEDIVQ
TLTLFEDREMIKKCLDIYKDFFTESQLKKLYRRHYTGWGRLSAKLINGIRNKENQKTILDYL
IDDGSANRNEMQLINDDDLSFKPIIDKARTGSHSDNLKEVVGELAGSPAIKKGILQSLKIVD
ELVKVMGYEPEQIVVEMARENQTTAKGLSRSRQRLTTLRESLANLKSNILEEKKPKYVKDQV
ENHHLSDDRLFLYYLQNGRDMYTKKALDIDNLSQYDIDHIIPQAFIKDDSIDNRVLVSSAKN
RGKSDDVPSIEIVKARKMFWKNLLDAKLMSQRKYDNLTKAERGGLTSDDKARFIQRQLVETR
QITKHVARILDERFNNEVDNGKKICKVKIVTLKSNLVSNFRKEFGFYKIREVNDYHHAHDAY
LNAVVAKAILTKYPQLEPEFVYGMYRQKKLSKIVHEDKEEKYSEATRKMFFYSNLMNMFKRV
VRLADGSIVVRPVIETGRYMGKTAWDKKKHFATVRKVLSYPQNNIVKKTEIQTGGFSKESIL
AHGNSDKLIPRKTKDIYLDPKKYGGFDSPIVAYSVLVVADIKKGKAQKLKTVTELLGITIME
RSRFEKNPSAFLESKGYLNIRDDKLMILPKYSLFELENGRRRLLASAGELQKGNELALPTQF
MKFLYLASRYNESKGKPEEIEKKQEFVNQHVSYEDDILQLINDESKRVILADANLEKINKLY
QDNKENIPVDELANNIINLFTFTSLGAPAAFKFFDKIVDRKRYTSTKEVLNSTLIHQSITGL
YETRIDLGKLGED
Streptococcus agalactiae Cas9 nuclease, DNA
SEQ ID NO: 242
ATGAACAAGCCTTATTCAATAGGATTAGATATAGGGACAAATTCTGTGGGGTGGAGTATAAT
CACCGACGATTACAAGGTGCCTGCAAAGAAGATGCGCGTGCTCGGCAATACAGACAAGGAAT
ATATTAAGAAGAACCTGATCGGGGCCCTCCTTTTTGACGGTGGCAACACAGCAGCTGACCGC
CGCCTCAAGAGGACCGCTCGGAGACGGTATACTCGCCGGCGTAATCGGATCCTGTATTTGCA
GGAAATTTTTGCTGAAGAAATGTCTAAGGTGGATGATTCATTCTTTCACCGGCTCGAAGACT
CCTTTCTGGTGGAGGAAGACAAGAGGGGCTCAAAGTACCCAATCTTCGCCACAATGCAAGAA
GAGAAATACTACCACGAGAAGTTTCCCACAATCTATCATCTCAGGAAAGAGCTGGCCGATAA
AAAAGAGAAGGCCGATTTGCGACTGGTTTACTTGGCCTTGGCACACATCATAAAGTTCCGGG
GACACTTTCTGATTGAAGACGACCGTTTTGACGTCCGCAACACTGATATACAGAAGCAATAC
CAAGCGTTCCTTGAGATCTTTGACACCACATTTGAAAACAACCATCTGCTGAGCCAAAATGT
GGACGTGGAAGCCATTCTGACTGATAAGATCTCTAAATCTGCCAAAAAGGACAGAATCCTTG
CCCAGTACCCCAACCAGAAGTCAACTGGCATTTTCGCCGAGTTTCTGAAGTTGATAGTTGGC
AATCAGGCCGATTTTAAGAAGCACTTCAATTTGGAGGACAAAACGCCTCTCCAATTCGCCAA
GGACTCATATGATGAGGACCTGGAGAATCTGCTTGGCCAAATCGGGGATGAGTTCGCTGATC
TTTTTAGCGTGGCAAAGAAGCTCTATGACTCTGTACTCCTGAGCGGAATCCTGACAGTTACC
GATCTTTCAACAAAGGCACCCCTGAGTGCAAGCATGATTCAACGCTACGACGAGCACCATGA
GGATCTGAAACATCTGAAGCAGTTCGTCAAGGCTTCTCTGCCTGAAAACTATCGGGAGGTCT
TCGCCGACTCATCTAAGGACGGCTACGCCGGATACATCGAGGGAAAGACAAATCAGGAGGCT
TTCTACAAGTACCTGTTGAAGCTGCTTACAAAACAGGAGGGGAGCGAATACTTCCTGGAGAA
GATCAAAAACGAGGACTTCCTGCGTAAACAGAGGACTTTCGATAATGGCTCCATTCCTCACC
AGGTGCATCTCACGGAACTGAGAGCTATCATTAGACGTCAGAGTGAGTATTACCCATTTCTG
AAGGAGAACCAAGACCGAATCGAAAAAATTCTGACGTTCCGGATCCCTTACTATGTCGGACC
TTTAGCTAGGGAAAAAAGTGACTTCGCCTGGATGACCCGAAAGACAGATGATAGTATCAGAC
CATGGAACTTTGAAGACCTGGTGGACAAAGAGAAGAGCGCCGAGGCTTTTATTCACAGGATG
ACCAATAATGATCTCTATCTGCCTGAAGAGAAGGTGCTGCCCAAACACAGTCTCATCTACGA
AAAATTTACAGTCTATAACGAACTGACAAAGGTCCGCTTTCTGGCTGAAGGATTCAAGGACT
TTCAATTTCTGAACCGGAAGCAGAAGGAAACTATCTTTAACTCATTGTTTAAGGAAAAGAGG
AAGGTTACCGAAAAAGACATCATCTCCTTTTTAAACAAGGTAGATGGGTACGAAGGGATTGC
CATTAAAGGCATTGAGAAACAGTTTAACGCCAGCCTTTCAACCTACCATGATCTCAAGAAGA
TCCTCGGAAAAGATTTCCTTGACAATACCGACAACGAACTTATCCTGGAGGATATAGTGCAG
ACACTCACTCTGTTCGAGGACAGGGAAATGATAAAGAAGTGCCTCGACATATATAAAGACTT
CTTTACCGAGAGTCAACTGAAAAAGTTGTATAGAAGGCATTACACCGGTTGGGGCCGACTGA
GTGCAAAACTCATTAACGGCATCCGGAATAAGGAGAATCAAAAGACTATCCTCGATTACCTC
ATCGATGACGGAAGCGCAAACAGAAACTTCATGCAACTCATCAACGATGATGACCTGTCTTT
CAAACCAATTATAGACAAAGCCAGGACTGGGAGCCATAGTGACAATCTGAAGGAAGTGGTGG
GAGAGCTGGCAGGCAGCCCCGCAATTAAGAAGGGGATCCTGCAGAGCCTCAAAATTGTCGAT
GAACTCGTGAAGGTCATGGGCTATGAACCTGAACAGATTGTTGTAGAGATGGCCCGAGAGAA
CCAGACTACTGCGAAGGGACTTAGCCGGAGCAGACAACGACTGACCACTTTGCGAGAGAGTC
TGGCGAACCTGAAGTCTAATATTCTCGAGGAAAAAAAGCCAAAGTACGTGAAGGACCAGGTG
GAGAATCACCACCTGAGCGACGACAGACTCTTTCTGTATTATCTGCAGAACGGCAGAGATAT
GTATACGAAGAAGGCACTGGACATAGACAACCTGAGTCAGTATGACATCGATcaCATTATCC
CTCAGGCCTTCATCAAAGACGATTCAATCGACAATCGCGTACTTGTTAGCAGTGCGAAAAAC
CGGGGAAAGTCTGATGACGTCCCATCCATCGAAATAGTGAAGGCAAGGAAGATGTTCTGGAA
GAATCTGCTGGATGCCAAATTAATGTCACAACGGAAGTACGACAACCTGACAAAGGCAGAAA
GGGGGGGCTTAACAAGCGACGATAAGGCAAGGTTTATCCAGAGGCAGTTGGTCGAGACCAGG
CAAATCACCAAACACGTCGCCCGGATCCTGGATGAACGCTTCAACAATGAAGTCGACAATGG
CAAAAAAATCTGTAAAGTCAAGATAGTGACACTGAAGTCAAATCTGGTGAGCAACTTCCGGA
AAGAATTCGGCTTCTATAAAATTCGCGAAGTGAACGACTATCACCATGCGCACGACGCTTAC
CTGAATGCAGTCGTGGCGAAAGCCATTTTGACCAAGTACCCCCAGCTGGAGCCTGAGTTTGT
GTACGGAATGTACCGACAAAAGAAGCTGAGCAAGATTGTACACGAGGATAAGGAAGAGAAAT
ACTCCGAGGCCACTCGGAAGATGTTCTTCTACTCTAATCTGATGAACATGTTTAAGAGAGTG
GTGAGGTTGGCAGACGGCTCCATTGTTGTAAGGCCAGTGATCGAGACTGGGCGATACATGGG
CAAGACAGCGTGGGACAAGAAGAAGCATTTCGCAACCGTACGGAAAGTCCTGTCCTACCCGC
AGAATAACATTGTGAAGAAGACAGAAATACAAACCGGTGGTTTCTCAAAAGAGTCCATTTTA
GCCCATGGCAACAGTGACAAATTGATTCCACGGAAGACCAAAGATATTTATCTGGACCCTAA
AAAATACGGCGGATTCGACTCACCGATCGTGGCATACAGCGTATTGGTGGTGGCCGATATTA
AGAAGGGTAAAGCCCAGAAACTCAAGACTGTTACCGAGCTCCTGGGTATCACTATAATGGAG
AGAAGCCGGTTTGAGAAGAACCCTAGCGCCTTTTTGGAATCCAAGGGGTATCTGAACATTCG
GGACGATAAGCTGATGATCTTGCCTAAATACAGCCTTTTTGAACTGGAGAATGGACGAAGGC
GCCTGCTTGCCTCAGCGGGGGAACTGCAGAAAGGCAATGAGCTGGCCCTTCCTACCCAGTTC
ATGAAATTTTTGTATCTGGCTAGTAGGTATAACGAGTCAAAAGGCAAGCCAGAGGAGATCGA
AAAGAAGCAGGAATTTGTAAACCAGCATGTGTCATACTTTGATGATATCCTGCAGTTAATCA
ATGACTTCAGTAAACGAGTCATTCTCGCAGACGCCAACTTGGAGAAAATTAATAAGCTGTAC
CAGGACAACAAAGAGAATATACCAGTCGACGAGCTTGCAAATAACATTATTAACCTGTTCAC
TTTTACATCCCTGGGGGCCCCTGCTGCGTTCAAATTTTTCGACAAAATCGTGGATCGAAAGC
GATATACATCCACTAAGGAAGTTCTGAACAGCACTCTCATCCACCAGTCTATCACTGGCCTT
TACGAAACGCGTATTGACTTGGGGAAACTCGGAGAGGAC
Streptococcus gallolyticus Cas9 nuclease, protein
SEQ ID NO: 243
MTNGKILGLDIGIASVGVGIIEAKTGKVVHANSRLFSAANAENNAERRGFRGSRRLNRRKKH
RVKRVRDLFEKYEIVTDFRNLNLNPYELRVKGLTEQLTNEELFAALRTISKRRGISYLDDAE
DDSTGSTDYAKSIDENRRLLKTKTPGQIQLERLEKYGQLRGNFTVYDENGEAHRLINVESTS
DYEKEARKILETQADYNKKITAEFIDDYVEILTQKRKYYHGPGNEKSRTDYGRFRTDGTTLE
NIFGILIGKCSFYPDEYRASKASYTAQEYNFLNDLNNLKVPTETGKLSTEQKEALVEFAKST
ATLGPAKLLKEIAKILDCKVDEIKGYREDDKGKPDLHTFEPYRKLKFNLDSVNIDDLSREVL
DKLADILTLNTEREGIEDAIRHNLPNQFTEGQISEIIKVRKSQSTAFNKGWHSFSAKLMNEL
IPELYATSDEQMTILTRLEKFKVNKKSSKNTKTIDEKEVTDEIYNPVVAKSVRQTIKIINAA
VKKYGDFDKIVIEMPRDKNADDEKKFIDKRNKENKKEKDDALKRAAYLYNGTDKLPDEVEHG
NKQLETKIRLWYQQGERCLYSGKPIPIQELVHNSNNFEIDHILPLSLSFDDSLANKVLVYAW
TNQEKGQKTPYQVIDSMDAAWSFREMKDYVLKQKGLGKKKRDYLLTTENIDKIEVKKKFIER
NLVDTRYASRVVLNSLQSALRELGKDTKISVIRGQFTSQLRRKWKIDKSRETYHHHAVDALI
IAASSQLKLWEKQDNPMFVDYGNNQVVDKQTGEILSVSDDEYKELVFQPPYQGFVNMISSKG
FEDEILFSYQVDSKYNRKVSDATIYSTRKAKIGKDKKEETYVLGKIKDIYSQNGFEDTFIKKY
NKDKTQFLMYQKDPLTWENVIEVILRDYPTTKKSEDGKNDVKCNPFEEYRRENGLVCKYSKK
GKGTPIKSLKYYDKKLGNCIDITPEGSKNEVVLQSLNPWRADVYFNPETLKYELLGLKYSDL
SFEKGTGKYHISQEKYDVIKEKEGIGKKSEFKFTLYRNDLILIKDTASGEQEIYRFLSRTMP
NVKHYAELKPYDKEKFDNVQELVEALGEADKVGRCIKGLNKSNLSIYKVRTDVLGNKYFVKK
EGDKPKLDFKNNKKTG
Streptococcus gallolyticus Cas9 nuclease, DNA
SEQ ID NO: 244
ATGACAAACGGCAAAATTCTGGGTCTGgatATCGGAATCGCTAGCGTTGGCGTGGGAATCAT
TGAAGCGAAGACAGGTAAAGTCGTCCATGCAAATTCTCGATTGTTCTCCGCAGCTAACGCTG
AAAACAATGCGGAGAGAAGGGGTTTCAGAGGCTCTAGGCGGCTCAACCGGCGCAAGAAGCAC
AGGGTAAAAAGAGTGCGAGATCTCTTTGAGAAATATGAGATCGTGACTGATTTTAGAAACCT
GAATCTGAACCCATATGAGCTGAGAGTGAAAGGACTTACGGAACAGCTCACTAATGAAGAGT
TGTTCGCCGCCCTGCGGACCATCAGCAAACGCCGAGGAATTTCCTACCTTGATGACGCCGAA
GATGACAGTACCGGTAGCACAGATTATGCCAAGAGCATTGATGAGAACAGGAGACTGCTGAA
GACTAAGACACCTGGACAGATACAATTGGAACGGCTCGAGAAGTACGGCCAGCTGAGGGGTA
ACTTCACCGTTTATGACGAAAATGGGGAGGCCCATAGACTGATAAATGTGTTCTCAACTTCT
GACTATGAAAAGGAGGCCCGGAAAATCCTCGAGACTCAAGCCGACTACAACAAGAAGATTAC
AGCCGAGTTTATTGACGATTACGTGGAAATTTTAACCCAGAAAAGGAAGTATTACCACGGGC
CAGGAAATGAAAAGAGCCGCACCGACTATGGGAGATTCAGAACGGATGGAACAACCTTAGAG
AATATCTTTGGAATCCTTATTGGTAAATGCTCTTTCTATCCTGACGAGTATCGCGCCAGCAA
AGCCTCCTATACCGCTCAGGAGTACAACTTCTTGAATGATTTGAACAATTTGAAGGTTCCGA
CGGAGACTGGCAAGCTGAGTACCGAGCAAAAGGAGGCCCTTGTGGAATTCGCCAAGTCTACT
GCAACATTAGGTCCTGCTAAACTTCTGAAGGAGATTGCCAAAATTTTGGACTGCAAAGTCGA
TGAAATCAAGGGGTACCGTGAGGATGATAAAGGGAAACCAGACCTGCACACCTTTGAGCCCT
ATAGAAAGTTGAAATTCAATCTGGACAGCGTCAACATTGACGATTTGAGTCGCGAAGTGCTG
GACAAGCTGGCAGACATTTTGACACTTAACACTGAAAGGGAGGGCATTGAGGATGCCATCAG
GCATAACCTGCCCAACCAATTTACTGAGGGCCAGATCTCCGAAATCATCAAGGTGCGCAAAA
GCCAGAGCACTGCTTTCAACAAGGGGTGGCACAGCTTCTCTGCCAAGCTCATGAACGAATTG
ATTCCCGAGCTCTATGCCACAAGCGACGAACAGATGACTATACTTACTCGGCTGGAGAAATT
TAAGGTCAATAAAAAATCCTCCAAAAACACCAAGACGATTGACGAGAAAGAGGTCACTGATG
AAATCTACAATCCAGTTGTAGCCAAGTCTGTCCGGCAAACGATCAAGATCATTAACGCTGCT
GTGAAGAAATATGGAGACTTTGATAAGATTGTGATTGAAATGCCTCGCGACAAGAATGCGGA
CGATGAGAAGAAGTTTATCGATAAGAGAAACAAAGAAAATAAGAAAGAAAAGGATGATGCCC
TGAAGCGGGCAGCTTACCTTTATAATGGAACCGATAAGCTGCCAGATGAGGTGTTTCACGGA
AACAAGCAACTTGAAACCAAGATTCGCCTGTGGTACCAGCAGGGAGAACGGTGTTTGTACTC
AGGCAAGCCTATCCCAATCCAGGAGTTGGTCCACAACTCCAATAACTTCGAAATCGATcacA
TTCTGCCCCTGTCCCTGAGTTTTGACGACTCCCTGGCCAACAAGGTGCTTGTGTATGCTTGG
ACCAACCAAGAGAAGGGCCAGAAGACGCCCTACCAGGTGATTGATTCTATGGATGCGGCGTG
GTCCTTTCGCGAGATGAAGGACTATGTGCTCAAGCAAAAAGGCCTCGGCAAAAAGAAACGGG
ATTATCTTTTGACCACCGAGAACATTGACAAGATTGAAGTGAAGAAAAAATTCATCGAGCGC
AACTTGGTCGATACCAGATATGCCTCTAGGGTTGTGCTGAACTCACTGCAGTCTGCTTTGAG
AGAGCTGGGTAAAGACACTAAAATTAGTGTAATCAGGGGCCAGTTCACAAGTCAGCTTAGGC
GGAAATGGAAGATCGACAAGTCACGCGAGACATATCATCATCACGCAGTCGACGCACTGATA
ATTGCAGCTTCAAGTCAGCTCAAGTTGTGGGAGAAACAGGATAACCCTATGTTTGTCGACTA
TGGAAACAATCAGGTCGTCGATAAGCAGACCGGGGAAATTTTAAGTGTGTCCGATGACGAGT
ATAAGGAGCTTGTCTTTCAGCCACCGTACCAGGGCTTTGTCAACATGATTAGTAGCAAGGGT
TTTGAGGACGAAATTTTGTTCAGCTACCAGGTCGATTCCAAATACAATAGAAAAGTATCCGA
CGCAACCATATATTCTACTCGCAAGGCCAAGATTGGCAAAGATAAGAAGGAAGAGACCTATG
TATTGGGGAAGATCAAAGACATTTACTCACAAAATGGATTCGACACCTTCATTAAGAAGTAC
AACAAAGATAAGACACAGTTTTTGATGTACCAGAAAGATCCACTGACATGGGAAAACGTGAT
CGAAGTTATACTGCGTGACTACCCCACGACTAAAAAGAGTGAGGACGGAAAAAACGACGTGA
AGTGCAACCCGTTTGAAGAATACCGGAGAGAAAACGGTCTGGTGTGTAAGTACTCTAAGAAA
GGAAAGGGGACCCCTATTAAATCCCTCAAATACTACGACAAAAAACTCGGGAACTGCATCGA
TATCACCCCGGAAGGTTCCAAAAATGAAGTCGTGCTTCAATCCTTGAATCCGTGGAGGGCAG
ATGTGTACTTTAACCCAGAAACCTTGAAGTATGAATTACTGGGACTTAAATACAGTGATCTC
TCATTTGAAAAGGGCACTGGAAAATACCATATCTCTCAGGAGAAGTACGACGTCATTAAGGA
AAAAGAAGGGATCGGGAAAAAATCCGAGTTCAAGTTCACATTGTATAGGAACGACCTGATCC
TTATTAAAGACACAGCCAGCGGTGAGCAGGAGATTTACCGATTTCTGTCTAGAACCATGCCT
AACGTCAAGCACTATGCGGAGCTGAAGCCCTATGACAAAGAAAAATTTGATAACGTCCAGGA
ACTCGTCGAGGCGCTGGGCGAAGCCGACAAGGTAGGCCGCTGTATAAAGGGGCTGAACAAAA
GCAACCTCAGCATCTATAAAGTTAGGACAGATGTGCTCGGGAACAAATACTTCGTTAAGAAG
GAAGGGGACAAGCCCAAGCTGGATTTTAAGAACAATAAAAAGACCGGT
SEQ ID NO: 245
Streptococcus iniae Cas9 nuclease, protein
MRKPYSIGLDIGTNSVGWAVITDDYKVPSKKMRIQGTTDRTSIKKNLIGALLFDNGETAEAT
RLKRTTRRRYTRRKYRIKELQKIFSSEMNELDIAFFPRLSESFLVSDDKEFENHPIFGNLKD
EITYHNDYPTIYHLRQTLADRDQKADLRLIYLALAHIIKFRGHFLIEGNLDSENTDVHVLEL
NLVNIYNNLFEEDIVETASIDAEKILTSKTSKSRRLENLIAEIPNQKRNMLFGNLVSLALGL
TPNFKTNFELLEDAKLQISKDSYEEDLDNLLAQIGDQYADLFIAAKKLSDAILLSDIITVKG
ASTKAPLSASMVQRYEEHQQDLALLKNLVKKQIPEKYKEIFDNKEKNGYAGYIDGKTSQEEF
YKYIKPILLKLNGTEKLISKLEREDELRKQRTFDNGSIPHQIHLNELKAIIRRQEKFYPFLK
ENQKKIEKLFTFKIPYYVGPLANGQSSFAWLKRQSNESITPWNFEEVVDQEASARAFIERMT
NEDTYLPEEKVLPKHSPLYEMEMVYNELTKVKYQTEGMKRPVELSSEDKEEIVNLLFKKDRK
VTVKQLKEEYFSKMKCFHTVTILGVEDRFNASLGTYHDLLKIFKDKAFLDDEANQDILEEIV
WTLTLFEDQAMIERRLVKYADVFEKSVLKKLKKRHYTGWGRLSQKLINGIKDKQTGKTILGF
LKDDGVANRNFMQLINDSSLDFAKIIKHEQEKTIKNESLEETIANLAGSPAIKKGILQSIKI
VDEIVKIMGQNPDNIVIEMARENQSTMQGIKNSRQRLRKLEEVHKNTGSKILKEYNVSNTQL
QSDRLYLYLLQDGKDMYTGKELDYDNLSQYDIDHIIPQSFIKDNSIDNIVLTTQASNRGKSD
NVPNIEIVNKMKSFWYKQLKNGAISQRKFDHLTKAERGALSDEDKAGFIKRQLVETRQITKH
VAQILDSRFNSNLTEDSKSNRNVKIITLKSKMVSDERKDFGFYKLREVNDYHHAQDAYLNAV
VGTALLKKYPKLEAEFVYGDYKHYDLAKLMIQPDSSLGKATTRMFFYSNLMNFFKKEIKLAD
DTIFTRPQIEVNTETGEIVWDKVKDMQTIRKVMSYPQVNIVMKTEVQTGGFSKESILPKGNS
DKLIARKKSWDPKKYGGFDSPIIAYSVLVVAKIAKGKTQKLKTIKELVGIKIMEQDEFEKDP
IAFLEKKGYQDIQTSSIIKLPKYSLFELENGRKRLLASAKELQKGNELALPNKYVKFLYLAS
HYTKFTGKEEDREKKRSYVESHLYYFDEIMQIIVEYSNRYILADSNLIKIQNLYKEKDNESI
EEQAINMLNLFTFTDLGAPAAFKFENGDIDRKRYSSTNEIINSTLIYQSPTGLYETRIDLSK
LGGK
Streptococcus iniae Cas9 nuclease, DNA
SEQ ID NO: 246
ATGCGCAAACCTTACTCAATTGGCCTGgatATCGGGACTAATTCTGTTGGCTGGGCTGTGAT
TACTGATGATTACAAGGTGCCAAGTAAGAAAATGAGGATTCAGGGCACGACTGATCGGACCA
GCATTAAGAAGAATCTCATTGGGGCCCTCCTGTTCGATAATGGCGAGACTGCCGAGGCCACT
CGATTAAAGAGAACAACAAGGAGGAGGTACACCAGACGGAAGTACCGAATAAAGGAACTGCA
AAAGATCTTCAGCAGCGAAATGAATGAGCTCGACATTGCTTTTTTCCCTAGACTGTCTGAGA
GTTTTCTTGTGAGTGACGACAAAGAATTCGAGAATCATCCGATTTTTGGAAACCTTAAAGAT
GAGATAACTTATCATAACGATTACCCTACTATTTATCACTTGCGACAGACACTTGCAGACCG
TGACCAGAAGGCCGATCTTAGGCTCATTTATCTCGCTCTGGCCCACATTATTAAATTTCGGG
GGCACTTTTTGATCGAAGGCAATCTGGACAGTGAGAACACGGACGTACACGTGCTGTTTCTG
AACCTGGTGAACATATATAATAACCTGTTCGAGGAAGATATAGTTGAAACCGCATCCATAGA
CGCTGAGAAGATTCTTACCTCAAAAACTTCCAAATCCAGGCGGCTCGAGAATCTTATAGCTG
AGATTCCTAACCAGAAGCGGAACATGTTGTTTGGCAACCTCGTGTCTCTGGCTCTCGGCCTG
ACACCAAATTTTAAAACCAATTTTGAGCTGCTGGAGGATGCAAAGTTACAGATCTCCAAGGA
TTCATATGAAGAAGACCTCGACAACTTGTTGGCACAGATTGGGGATCAGTACGCAGATCTCT
TTATCGCCGCTAAAAAGCTTTCTGACGCAATATTACTGTCTGACATCATCACCGTGAAGGGC
GCCTCCACTAAAGCGCCTCTTTCAGCATCCATGGTGCAGAGATATGAAGAGCATCAACAGGA
CCTCGCTCTCCTGAAGAATCTCGTGAAAAAACAGATTCCTGAGAAGTATAAGGAAATCTTCG
ATAACAAGGAGAAGAATGGCTATGCAGGTTATATCGATGGCAAGACCTCCCAGGAGGAATTT
TACAAGTACATCAAGCCCATACTTCTTAAGCTCAACGGCACAGAGAAGTTGATCAGCAAACT
TGAGCGGGAGGACTTCCTGAGAAAGCAACGAACATTCGACAACGGATCTATTCCTCACCAGA
TTCACCTGAATGAGCTCAAGGCAATCATCCGGAGGCAGGAGAAGTTTTATCCCTTTCTGAAG
GAAAATCAGAAGAAAATCGAAAAGCTTTTCACATTTAAAATTCCCTATTACGTCGGGCCACT
CGCCAATGGCCAGAGTAGCTTCGCCTGGCTGAAGAGACAGTCCAACGAGTCTATCACCCCCT
GGAACTTCGAGGAAGTGGTGGATCAAGAGGCCTCAGCGCGCGCCTTCATAGAGAGGATGACT
AACTTCGATACCTATTTACCCGAGGAGAAGGTTCTGCCAAAGCACAGCCCACTCTACGAAAT
GTTTATGGTCTATAATGAGCTCACCAAGGTTAAGTATCAGACCGAGGGGATGAAGAGGCCCG
TCTTTCTCTCTTCCGAAGACAAAGAAGAAATAGTGAATCTCCTGTTCAAAAAAGACCGGAAG
GTCACTGTCAAGCAGCTGAAGGAGGAATATTTCTCCAAAATGAAATGCTTCCACACCGTGAC
AATCTTGGGCGTGGAGGATCGGTTTAATGCTTCTCTGGGCACGTACCATGACCTGCTCAAAA
TTTTTAAAGATAAAGCCTTCTTAGACGATGAGGCCAATCAAGATATCTTGGAAGAGATCGTA
TGGACTTTAACGCTTTTTGAGGATCAAGCCATGATTGAAAGAAGGCTGGTGAAGTACGCGGA
CGTGTTCGAAAAATCCGTCCTTAAAAAGTTAAAGAAACGCCATTACACGGGCTGGGGACGTC
TTTCCCAGAAGCTTATTAATGGGATCAAAGACAAACAAACTGGGAAGACAATTCTCGGCTTT
CTGAAAGACGACGGTGTAGCCAACCGAAATTTTATGCAGTTAATTAACGACAGCTCCCTGGA
CTTCGCAAAGATTATCAAGCATGAACAGGAAAAAACCATCAAGAACGAGTCATTGGAGGAAA
CGATTGCGAACCTGGCAGGCAGCCCCGCCATTAAGAAAGGCATTCTTCAGTCTATTAAAATT
GTCGATGAAATCGTTAAGATTATGGGACAGAACCCAGACAATATTGTTATTGAGATGGCACG
CGAGAACCAATCCACGATGCAAGGAATCAAAAACTCCCGACAGCGTCTGCGCAAGCTCGAGG
AGGTGCATAAGAACACCGGGTCCAAGATTTTGAAAGAATACAACGTGAGTAATACGCAGCTT
CAGAGCGATAGGCTCTATTTATACCTGCTGCAGGACGGAAAGGATATGTACACCGGCAAGGA
GTTGGACTACGACAATCTTAGTCAATATGATATTGATcacATCATCCCTCAGTCTTTCATAA
AAGATAACTCTATCGACAACATAGTGCTGACTACACAAGCTAGTAATAGGGGCAAGTCAGAC
AACGTGCCCAACATAGAGATTGTGAACAAAATGAAGTCTTTTTGGTATAAACAGCTCAAAAA
TGGGGCAATTAGCCAGCGCAAATTCGACCATTTAACCAAGGCCGAGCGTGGCGCACTGAGCG
ATTTCGATAAGGCAGGCTTTATCAAGCGCCAGCTCGTCGAGACACGGCAGATAACCAAACAT
GTGGCTCAAATCCTGGACAGTCGGTTCAATTCCAATCTTACGGAGGACTCTAAATCTAACAG
AAACGTTAAGATAATAACTCTCAAGTCAAAAATGGTGAGTGACTTCCGAAAGGACTTTGGCT
TTTACAAGCTGAGAGAAGTAAATGATTATCACCACGCCCAGGACGCATATCTCAATGCCGTC
GTCGGTACTGCCTTACTTAAGAAGTACCCTAAACTGGAAGCAGAGTTCGTGTATGGGGATTA
CAAGCACTACGATCTCGCTAAGTTAATGATTCAACCGGACAGTAGCCTTGGAAAAGCCACAA
CCAGAATGTTCTTCTATTCTAACCTCATGAATTTCTTCAAAAAAGAAATCAAACTGGCCGAT
GATACTATATTTACGAGGCCCCAGATTGAAGTGAACACCGAAACTGGGGAGATTGTCTGGGA
TAAGGTAAAGGACATGCAGACCATCAGGAAAGTGATGTCCTATCCACAAGTCAACATAGTGA
TGAAAACCGAAGTCCAGACTGGGGGGTTTTCTAAGGAGAGTATCCTGCCTAAGGGAAACTCA
GACAAACTGATCGCCCGCAAGAAATCCTGGGACCCTAAGAAATACGGTGGTTTCGATAGCCC
TATCATTGCATATTCAGTCCTGGTCGTCGCTAAGATAGCCAAAGGCAAAACCCAGAAACTCA
AGACTATTAAAGAGTTGGTCGGTATCAAAATCATGGAGCAGGACGAATTCGAAAAGGATCCA
ATTGCGTTTCTCGAAAAGAAGGGCTATCAGGACATACAGACCTCTTCCATCATCAAGCTGCC
GAAGTACTCTCTCTTTGAGCTTGAGAATGGACGCAAGAGACTGCTGGCTAGCGCCAAAGAAC
TGCAGAAGGGCAACGAACTGGCCCTCCCTAACAAATACGTAAAGTTCTTGTATTTAGCATCT
CATTACACAAAATTCACAGGTAAGGAGGAAGATCGAGAAAAAAAGCGCTCCTATGTAGAGTC
ACACCTGTATTACTTTGACGAGATTATGCAGATTATCGTTGAGTATTCTAACCGGTACATTC
TCGCCGACAGCAATCTGATTAAAATTCAGAACTTGTACAAAGAGAAGGATAACTTTAGTATC
GAGGAGCAAGCCATTAATATGCTCAATCTCTTCACTTTTACAGATCTCGGCGCGCCAGCCGC
TTTCAAGTTCTTTAACGGAGATATAGATCGGAAGCGGTACAGCTCTACCAACGAGATCATTA
ATTCTACTCTGATTTACCAGAGTCCCACAGGGTTATACGAGACCAGGATCGACCTCAGTAAG
CTGGGGGGCAAA
Streptococcus lutetiensis Cas9 nuclease, protein
SEQ ID NO: 247
MSNGKILGLDIGVASVGVGIIDAKTGNVIHANSRLFSAANAENNAERRGFRGARRLTRRKKH
RVKRVRDLFEKYDISTDERNLNLNPYELRVKGLTEQLTNEELFAALRTIAKRRGISYLDDAE
DDSTGSSDYAKSIDENRRLLKTKTPGQIQLERLEKYGQLRGNFTVYDENGEAHRLINVESTS
DYKNEARKILETQSNYNKQITDEFIEDYIEILTQKRKYYHGPGNEKSRTDYGRFRTDGTTLE
NIFGILIGKCSFYPEEYRASKASYTAQEFNFLNDLNNLKVPTETGKLSTEQKEYLVDFAKKS
KALGASKLLKEIAKIVDCSVDDIKGYRVDNKDKPDLHTFEPYRKLKENLSSIDIDELSRETL
DKLADILTLNTEREGIEDTIKRNLPSQFTEEQISEIVQIRKNQSSAFNKGWHSFSAKLMNEL
IPELYVTSEEQMTILTRLEKFKVNKKSSKNTKTIDEKEITDEIYNPVVAKSVRQTIKIINAA
VKKYGDFDKIVIEMPRDKNAEDEKKFIDKKEKENKKEKDDSLKRAAFLYNGTDNLPDGVFHG
NKELKTKIRLWYQQGERCLYSGKLISIHDLVHNSNKFEIDHILPLSLSFDDSLANKVLVYAW
TNQEKGQKTPYQVIDSMDAAWSFREMKDYVLKQKRLGKKKREYLLTTENIDKIEVKKKFIER
NLVDTRYASRVVLNSLQTALKELGKDTKVSVVRGQFTSQLRRKWNIDKSRETYHHHAVDALI
IAASSQLKLWQKQENPMFESYGENQVVNKETGEILSISDDKYKELVFQPPYQGFVNTISSKG
FEDEILFSYQVDSKFNRKVSDATIYSTRKAKLGKDKKDETYVLGKIKDIYSQDGEDTFIKRY
KKDKTQFLMYQKDPLTWENVIEVILRDYPSEKLSEDGKKTVKCNPFEEYRRENGLICKYSKK
GNGTPIKSLKYYDKKLGNCIDITPEKSKNRVVLRQISPWRADIYFNLETLKYELMGLKYSDL
SFEKGTGKYHISQEKYDAIREKEGIGKKSEFKFTLYRNDLILIKDTLNNCERMLRFGSKNDT
SKHYVELKPLEKGTFDSEEEILPVLGKVAKSGQFIKGLNKPNISIYKVRTDVLGNKFFIKKE
GDKPKLDFKNNNK
Streptococcus lutetiensis Cas9 nuclease, DNA
SEQ ID NO: 248
ATGTCAAATGGCAAAATCTTAGGCTTGgatATCGGGGTGGCCAGCGTCGGGGTTGGCATAAT
TGATGCCAAAACCGGCAACGTGATCCACGCAAATAGCAGGCTGTTTAGCGCCGCCAACGCCG
AGAACAATGCTGAGCGGAGGGGATTCCGCGGCGCACGTAGGCTCACGAGGCGCAAAAAACAT
AGAGTGAAGCGGGTCCGTGACCTGTTTGAAAAGTATGATATCTCAACAGATTTCCGCAACTT
AAATCTGAACCCCTACGAGCTCAGGGTGAAAGGCCTGACAGAACAGCTTACCAATGAAGAAC
TCTTCGCAGCTTTAAGAACTATTGCCAAACGGCGCGGCATCTCCTACTTGGATGACGCGGAA
GACGATTCTACCGGAAGCAGCGACTACGCGAAGTCAATCGACGAAAATAGACGTCTTCTGAA
AACCAAAACTCCAGGGCAAATCCAGCTGGAGAGACTGGAGAAGTACGGACAGCTGAGGGGCA
ATTTTACCGTGTATGACGAAAACGGAGAAGCTCACAGACTGATCAATGTTTTTTCCACTTCC
GATTATAAAAACGAAGCCCGGAAGATCCTGGAGACGCAGAGCAACTACAACAAGCAAATCAC
CGATGAGTTCATCGAAGATTACATTGAGATATTAACTCAAAAGCGTAAATACTACCATGGCC
CAGGCAACGAGAAGAGCAGGACCGATTACGGCAGGTTCCGAACAGATGGAACTACCCTGGAG
AACATTTTTGGCATTCTTATTGGAAAATGCTCATTCTATCCAGAGGAATATCGTGCTAGTAA
GGCAAGCTACACCGCCCAAGAATTCAACTTTCTGAATGACCTGAATAATCTGAAGGTCCCCA
CCGAAACGGGCAAGTTATCAACTGAGCAGAAGGAGTATTTAGTGGATTTTGCCAAGAAGTCT
AAGGCTCTGGGAGCGTCTAAGCTTCTGAAGGAGATTGCCAAGATAGTTGATTGCAGCGTTGA
CGACATCAAGGGGTACAGGGTGGATAATAAAGACAAGCCAGATCTGCACACCTTTGAGCCAT
ATAGAAAGTTGAAGTTTAACTTGAGTAGTATCGACATCGATGAACTGTCTAGAGAGACACTC
GACAAACTCGCTGACATTCTTACTCTGAACACAGAACGGGAAGGCATCGAGGATACAATCAA
AAGAAACCTTCCCTCACAGTTTACCGAGGAACAGATAAGCGAGATTGTCCAAATTCGGAAGA
ATCAATCCAGCGCCTTTAACAAGGGTTGGCACTCCTTCTCAGCAAAGTTGATGAACGAGTTA
ATCCCAGAGCTGTACGTGACTTCAGAGGAGCAGATGACAATTCTGACCAGGTTGGAAAAATT
TAAGGTGAACAAGAAGAGCTCCAAAAACACAAAGACCATCGATGAAAAGGAGATTACTGACG
AGATCTATAACCCAGTCGTCGCGAAATCCGTGAGGCAAACTATCAAGATTATCAACGCCGCG
GTGAAAAAGTATGGAGACTTTGACAAAATCGTGATTGAGATGCCACGTGACAAGAATGCAGA
GGATGAGAAAAAATTTATTGACAAAAAGGAGAAGGAAAATAAGAAGGAAAAAGATGATAGCC
TGAAGCGCGCAGCTTTCCTGTATAACGGCACAGACAATTTGCCAGACGGAGTATTTCACGGA
AACAAGGAGCTCAAGACTAAAATTCGCTTATGGTATCAACAAGGCGAGAGGTGCTTGTATAG
CGGCAAACTGATATCCATACACGACCTCGTACACAACAGTAACAAGTTTGAGATTGACcacA
TCCTTCCACTTAGCCTGAGTTTCGACGACAGCCTGGCAAATAAGGTCTTGGTATATGCTTGG
ACCAATCAGGAGAAGGGGCAAAAAACCCCGTACCAGGTGATAGATAGCATGGACGCGGCATG
GAGTTTTCGGGAAATGAAGGACTACGTTCTCAAACAGAAGAGACTCGGCAAAAAAAAGCGTG
AATACCTGCTGACTACCGAGAACATTGACAAAATCGAAGTCAAAAAAAAGTTCATCGAGCGC
AACCTTGTGGATACCCGCTATGCCTCACGCGTCGTCCTGAACTCTCTGCAGACAGCTCTGAA
AGAACTGGGCAAGGACACCAAAGTGTCTGTCGTTAGGGGTCAATTTACCTCCCAGTTGCGAC
GCAAGTGGAATATCGATAAGTCCAGAGAAACATACCATCATCACGCAGTAGACGCCCTTATC
ATTGCCGCATCTTCTCAGCTTAAACTGTGGCAAAAGCAGGAAAATCCTATGTTTGAGTCTTA
TGGCGAAAATCAGGTCGTCAATAAGGAGACAGGAGAGATCTTATCAATATCCGATGACAAGT
ATAAAGAACTGGTGTTTCAACCACCATACCAAGGGTTTGTCAACACTATCAGCAGTAAAGGC
TTCGAGGATGAGATCTTGTTTTCATATCAGGTGGACAGCAAATTCAACCGGAAAGTTTCTGA
TGCCACCATTTATAGTACTCGCAAAGCGAAACTTGGAAAGGACAAGAAGGATGAGACCTACG
TATTGGGGAAAATCAAGGACATTTACTCTCAGGACGGCTTTGACACCTTCATTAAGCGTTAC
AAAAAGGACAAGACGCAGTTCCTGATGTACCAAAAAGATCCACTGACTTGGGAAAATGTTAT
TGAGGTGATCCTCCGGGATTATCCAAGTGAAAAATTGTCAGAGGACGGCAAAAAAACAGTGA
AGTGCAATCCGTTTGAAGAATATAGGCGAGAGAATGGTCTGATCTGTAAATACTCTAAAAAG
GGCAACGGAACCCCCATCAAGTCCCTGAAATATTACGACAAGAAACTTGGTAACTGCATTGA
CATCACCCCTGAGAAAAGCAAGAACCGCGTGGTGCTGAGGCAGATATCACCTTGGCGCGCTG
ATATCTACTTCAACCTGGAGACCTTGAAATATGAGCTCATGGGCTTGAAATACAGTGACCTG
TCTTTTGAAAAAGGGACCGGGAAGTATCACATTAGCCAGGAAAAGTACGATGCGATTAGAGA
AAAAGAAGGCATTGGCAAAAAGAGCGAGTTTAAGTTTACTTTGTATCGAAACGATCTCATCC
TGATAAAAGATACCCTGAACAATTGTGAGAGGATGCTTAGGTTCGGATCCAAGAACGATACA
TCTAAGCACTACGTGGAACTCAAACCTTTAGAGAAGGGCACCTTTGATTCCGAGGAGGAGAT
CCTTCCAGTGCTGGGCAAGGTTGCGAAATCCGGGCAGTTTATTAAGGGTCTTAACAAACCCA
ATATCTCAATCTATAAGGTGAGGACCGATGTGCTTGGCAACAAATTCTTTATCAAGAAGGAA
GGCGACAAACCCAAGCTGGATTTCAAGAATAATAACAAG
Streptococcus mutans Cas9 nuclease, protein
SEQ ID NO: 249
MKKPYSIGLDIGTNSVGWAVVTDDYKVPAKKMKVLGNTDKSHIKKNLLGALLFDSGNTAADR
RLKRTARRRYTRRRNRILYLQEIFAEEMSKVDDSFFHRLEDSFLVTEDKRGERHPIFGNLEE
EVKYYENFPTIYHLRQYLADNPEKVDLRLVYLALAHIIKFRGHFLIEGKEDTRNNDVQRLFQ
EFLAVYDNTFENSSLQEQNVQVEEILTDKISKSAKKDRVLKLFPNEKSNGRFAEFLKLIVGN
QADFKKHFELEEKAPLQFSKDTYEEDLEELLGKIGDDYADLFTLAKNLYDAILLSGILTADD
SSTKAPLSASMIQRYNEHQMDLAQLKQFIRQKLSDKYNEVESDVSKDGYAGYIDGKTNQEAF
YKYLKGLLNKIEGSGYFLDKIEREDFLRKQRTFDNGSIPHQIHLQEMRAIIRRQAEFYPFLA
DNQDRIEKILTFRIPYYVGPLARGKSDFAWLSRKSADKITPWNFDEIVDKESSAEAFINRMT
NYDLYLPNQKVLPKHSLLYEKFTVYNELTKVKYKTEQGKTAFFDANMKQEIFDGVFKVYRKV
TKDKLMDFLEKEFDEFRIVDLTGLDKENKVFNASYGTYHDLCKILDKDFLDNSKNEKILEDI
VLTLTLFEDREMIRKRLENYSDLLTKEQVKKLERRHYTGWGRLSAELIHGIRNKESRKTILD
YLIDDGNSNRNEMQLINDDALSFKEEIAKAQVIGETDNLNQVVSDIAGSPAIKKGILQSLKI
VDELVKIMGHQPENIVVEMARENQFTNQGRRNSQQRLKGLTDSIKEFGSQILKEHPVENSQL
QNDRLFLYYLQNGRDMYTGEELDIDYLSQYDIDHIIPQAFIKDNSIDNRVLTSSKENRGKSD
DVPSKDVVRKMKPYWSKLLSAKLITQRKEDNLTKAERGGLTDDDKAGFIKRQLVETRQITKH
VARILDERENTETDENNKKIRQVKIVTLKSNLVSNFRKEFELYKVREINDYHHAHDAYLNAV
IGKALLGVYPQLEPEFVYGDYPHFHGHEENKATAKKFFYSNIMNFFKKDDVRTDKNGEIIWK
KDEHISNIKKVLSYPQVNIVKKVEEQTGGFSKESILPKGNSDKLIPRKTKKFYWDTKKYGGF
DSPIVAYSILVIADIEKGKSKKLKTVKALVGVTIMEKMTFERDPVAFLERKGYRNVQEENII
KLPKYSLFKLENGRKRLLASARELQKGNEIVLPNHLGTLLYHAKNIHKVDEPKHLDYVDKHK
DEFKELLDVVSNFSKKYTLAEGNLEKIKELYAQNNGEDLKELASSFINLLTFTAIGAPATEK
FFDKNIDRKRYTSTTEILNATLIHQSITGLYETRIDLSKLGGD
Streptococcus mutans Cas9 nuclease, DNA
SEQ ID NO: 250
ATGAAGAAGCCTTACTCAATTGGCCTGGATATTGGCACTAATTCAGTGGGATGGGCCGTCGT
TACCGATGATTACAAGGTACCCGCAAAGAAGATGAAGGTCCTTGGTAATACAGATAAAAGTC
ACATAAAGAAGAATCTTCTCGGAGCTCTTCTGTTCGACAGCGGGAACACAGCTGCCGATAGG
CGACTCAAAAGAACTGCTCGCAGGCGCTATACAAGGCGCAGAAACCGCATTCTGTACCTGCA
GGAGATCTTCGCCGAAGAGATGTCCAAAGTGGATGACAGTTTTTTCCATAGGCTCGAGGATA
GCTTCCTGGTGACCGAGGACAAAAGGGGGGAGAGACATCCCATTTTCGGTAATCTTGAAGAG
GAGGTTAAGTACTACGAGAACTTCCCGACTATATATCATCTGCGGCAGTATCTCGCAGACAA
CCCCGAAAAAGTGGACCTGCGACTTGTGTATCTTGCCCTGGCACATATTATAAAATTCAGAG
GCCACTTTCTGATTGAGGGGAAGTTTGATACCCGGAATAATGATGTGCAGCGCCTGTTTCAG
GAATTCTTGGCTGTCTACGACAATACATTTGAGAATAGTAGTTTGCAGGAGCAGAACGTGCA
AGTGGAAGAGATCCTGACAGACAAGATCTCCAAGAGCGCCAAAAAAGATAGGGTGCTCAAAT
TGTTCCCTAATGAGAAATCCAACGGCAGGTTTGCCGAATTCTTGAAACTGATTGTGGGAAAC
CAGGCTGACTTTAAGAAACATTTCGAGCTTGAAGAAAAGGCCCCTTTGCAGTTTTCCAAAGA
CACCTACGAGGAGGATCTGGAGGAACTGCTGGGAAAGATCGGGGATGACTATGCCGATCTGT
TTACCCTCGCCAAGAACCTGTACGATGCGATTCTCTTGTCCGGTATCCTGACGGCAGACGAC
AGTTCAACTAAAGCTCCGCTCTCTGCCAGCATGATTCAGCGATACAATGAGCATCAGATGGA
TCTGGCCCAGCTCAAGCAGTTCATCCGACAGAAACTCAGCGATAAGTACAACGAGGTGTTTA
GCGACGTGTCCAAAGACGGGTACGCAGGCTACATTGACGGCAAGACCAACCAAGAAGCGTTC
TACAAATACCTGAAAGGGCTGCTCAACAAGATAGAAGGATCAGGTTACTTTCTGGATAAAAT
CGAACGGGAGGATTTTTTGCGCAAGCAGCGAACTTTCGACAATGGGTCCATCCCTCATCAGA
TTCACCTGCAGGAAATGAGAGCTATTATTAGGAGACAGGCTGAATTTTACCCTTTTCTGGCA
GATAACCAGGATCGGATCGAGAAAATCTTAACCTTTCGGATCCCATACTATGTGGGCCCACT
GGCCCGTGGCAAATCCGACTTCGCATGGCTGTCACGGAAGTCCGCCGATAAAATTACGCCGT
GGAACTTTGATGAAATTGTCGATAAGGAATCTTCCGCTGAGGCTTTTATCAATCGCATGACC
AATTACGATCTGTACCTGCCTAATCAGAAGGTGTTACCCAAGCATAGCCTGTTGTATGAAAA
ATTCACTGTCTACAATGAACTCACCAAAGTCAAGTACAAGACAGAACAGGGCAAAACCGCCT
TTTTCGACGCTAATATGAAACAGGAAATTTTTGACGGGGTGTTCAAAGTCTATAGAAAGGTC
ACTAAGGACAAACTGATGGATTTTCTGGAGAAGGAATTTGATGAGTTTCGCATAGTTGATCT
TACTGGTTTGGATAAAGAAAATAAGGTCTTCAATGCAAGCTACGGTACATACCACGACCTTT
GTAAAATTCTCGACAAGGATTTCCTCGACAACTCCAAAAATGAAAAGATTCTTGAGGATATC
GTGTTAACCCTGACCCTGTTTGAAGACAGGGAAATGATCCGGAAGCGGCTGGAGAATTACTC
CGACCTGTTGACTAAAGAGCAGGTGAAAAAGCTCGAGAGGCGCCATTACACCGGATGGGGGA
GACTCAGTGCCGAACTTATCCATGGAATTCGAAACAAGGAGAGCAGGAAGACCATTCTCGAT
TATCTGATTGACGATGGTAATAGCAACAGAAATTTTATGCAGCTGATCAACGATGATGCACT
GTCATTTAAGGAGGAAATTGCAAAAGCCCAGGTTATCGGCGAGACCGACAACCTGAATCAGG
TTGTGAGTGACATCGCAGGGAGCCCCGCTATCAAGAAGGGAATCCTCCAGTCCCTCAAGATT
GTCGACGAGCTCGTCAAGATCATGGGGCATCAGCCAGAGAACATTGTCGTGGAGATGGCCCG
CGAAAACCAATTTACCAACCAAGGGAGGCGGAACAGCCAGCAAAGACTGAAGGGCTTAACAG
ATAGCATTAAAGAGTTCGGATCTCAGATACTTAAAGAACACCCCGTCGAAAACTCCCAGTTG
CAGAATGACCGCCTCTTTCTGTATTATCTGCAAAACGGAAGGGACATGTATACGGGAGAGGA
GCTGGATATAGATTACCTTAGTCAATATGATATCGATCacATAATCCCCCAAGCCTTTATCA
AGGACAACTCTATAGACAATAGGGTCCTGACCTCTAGCAAAGAGAATAGAGGCAAGTCCGAT
GACGTACCTTCTAAGGATGTCGTGCGCAAGATGAAGCCATACTGGAGCAAGCTGCTGTCTGC
AAAGCTTATAACCCAACGAAAGTTCGATAATCTGACTAAGGCCGAGCGCGGCGGGCTGACAG
ATGACGATAAGGCCGGGTTCATTAAGCGCCAGCTGGTGGAAACAAGACAAATCACTAAACAC
GTCGCTCGAATTCTTGATGAGCGGTTTAACACAGAAACGGACGAAAACAACAAAAAGATCCG
CCAGGTAAAAATTGTAACCCTGAAGAGCAACCTTGTTTCTAATTTCAGAAAGGAATTCGAAC
TTTACAAAGTGCGTGAAATCAACGACTACCATCATGCCCATGACGCTTATCTGAACGCTGTC
ATCGGGAAGGCCCTCCTTGGGGTCTATCCTCAGCTGGAGCCTGAATTTGTGTACGGAGATTA
CCCACACTTTCACGGGCACGAAGAGAACAAGGCAACTGCTAAGAAGTTCTTTTATTCAAATA
TCATGAATTTTTTTAAGAAAGACGACGTCAGAACTGATAAAAACGGTGAGATCATTTGGAAG
AAGGACGAACATATCAGTAATATTAAAAAGGTGCTTAGCTATCCTCAGGTGAACATAGTTAA
AAAAGTAGAGGAGCAGACAGGCGGGTTCTCCAAGGAATCCATACTGCCAAAAGGCAACAGCG
ATAAACTGATACCTCGGAAGACTAAAAAATTCTACTGGGATACCAAGAAGTACGGGGGATTT
GACAGCCCCATTGTCGCCTACTCTATATTGGTTATTGCGGACATCGAAAAGGGCAAATCCAA
AAAGCTTAAAACTGTCAAAGCCCTGGTGGGGGTTACCATCATGGAGAAAATGACCTTTGAAC
GCGATCCCGTAGCATTCCTCGAACGCAAGGGCTACCGCAACGTTCAGGAGGAGAATATCATC
AAGTTGCCCAAATATTCTCTCTTTAAGCTGGAGAACGGCAGAAAGCGCCTGCTCGCATCCGC
AAGGGAGTTACAGAAAGGCAACGAAATTGTACTGCCCAATCACCTCGGAACCCTGCTGTATC
ACGCCAAAAATATCCATAAAGTCGACGAACCTAAGCACTTAGACTATGTCGATAAACACAAG
GACGAATTTAAAGAGCTGCTGGACGTGGTTAGCAATTTCTCAAAGAAATACACGCTGGCGGA
AGGTAATCTGGAGAAAATTAAAGAGTTGTACGCTCAGAATAACGGGGAGGATCTTAAAGAAC
TGGCGTCCTCATTTATCAACCTGCTGACCTTCACCGCCATCGGCGCGCCTGCTACATTTAAA
TTCTTCGATAAGAATATCGATAGAAAGAGATATACTTCCACGACCGAAATTTTGAACGCTAC
CCTGATTCACCAATCTATTACCGGGTTATATGAGACACGAATTGATCTGAGCAAACTGGGGG
GGGAT
Streptococcus parauberis Cas9 nuclease, protein
SEQ ID NO: 251
MQKSYSLGLDIGTNSVGWAVITDDYKVPAKKMKVLGNTDRQTVKKNMIGTLLEDSGETAEAR
RLKRTARRRYTRRINRIKYLQSIFDDEMSKIDSAFFQRIKDSFLVPDDKNDDRHPIFGNIKD
EVDYHKNYPTIYHLRKKLADSDEKADLRLIYLALAHIIKERGHFLIEGDLDSQNTDVNALFL
KLVDTYNLMFEDDKIDTQTIDATVILTEKMSKSRRLENLIAKIPNQKKNTLFGNLISLSLGL
TPNFKANFELSEDAKLQISKDSFEEDLDNLLAQIGDQYADLFIAAKNLSDAILLSDILTVKG
VNTKAPLSASMVQRFNEHQDDLKLLKKLVKVQLPEKYKEIFDIKDKNGYAGYINGKTSQEDF
YKYIKPILSKLKGAESLISKLEREDFLRKQRTFDNGSIPHQIHLNELKSIIRRQEKYYPFLK
DKQVRIEKIFTFRIPYFVGPLANGNSSFAWVKRRSNESITPWNFEEVVEQEASAKVFIERMT
NFDTYLPEEKVLPKHSLLYEMFTVYNELTKVKYQAEGMRKPEFLSSEEKIEIVSNLEKKERK
VTVKQLKENYFNKIRCLDSITISGVEDKFNASLGTYHDLLNIIKNQKILDDEQNQDSLEDIV
LTLTLFEDEKMIAKRLSKYESIFEPSILKKLKKRHYTGWGRLSQKLINGIRDKQTGKTILDE
LIDDGQANRNFMQLINDPSLDFASIIKGAQEKTIKSEKLEETIANLAGSPAIKKGILQSVKI
VDEVVKVMGYEPSNIVIEMARENQSTHRGINNSRERLRKLEEVHKNIGSKILKEHEISNAQL
QSDRVYLYLLQDGKDMYTGKDLDFDRLSQYDIDHIIPQSFIKDNSIDNIVLTSQESNRGKSD
NVPYIAIVNKMKSYWQHQLKSGAISQRKEDNLTKVERGGLSEYDKAGFIKRQLVETRQITKH
VAQILNNRENNNVDNSSKNKRPVKIITLKSKMVSDFRKEFGFYKIREVNDYHHAHDAYLNAV
VGTALLKKYPKLEAEFVYGDYKHYDLASLVVKSDTSLGKATAKMFFYSNIMNFFKKEVRLAD
GTVITRPQIETNTETGEIVWDKVKDIKTIRKVLSIPQINVVKKTEVQTGGFSKESILPKGDS
DKLIPRKNNWDPKKYGGFDSPIIAYSVLVVAKVAKGKSQKTKSVKELVGITIMEQNEFEKDR
ITFLEKKGYQDIQESLIIKLPKFSLFELENGRKRLLASAKELQKGNELSLPNKYIQFLYLAS
RYTSFSGKEEDREKHRHEVESHLHYFDEIKDIIADESRRYILADANLEKILTLYNEKNQFSI
EEQATNMLNLFTFTGLGAPATLKFFNVDIDRKRYTSSTEILNSTLIRQSITGLYETRIDLSK
IGGD
Streptococcus parauberis Cas9 nuclease, DNA
SEQ ID NO: 252
ATGCAAAAGAGCTACTCTCTCGGGTTAGaCATCGGAACAAATAGTGTGGGATGGGCGGTGAT
TACGGACGATTATAAGGTGCCAGCCAAAAAGATGAAGGTTCTTGGCAATACGGACCGGCAGA
CGGTGAAGAAGAACATGATTGGCACTCTGCTGTTTGATAGTGGAGAAACCGCTGAGGCCCGG
AGACTCAAAAGGACTGCTAGGCGACGGTATACGCGGCGTATTAACCGCATTAAATATCTTCA
GTCTATATTTGATGATGAGATGTCAAAGATCGACAGCGCGTTTTTTCAGCGAATTAAAGATT
CCTTCCTTGTCCCAGATGACAAGAATGACGATAGACATCCGATTTTTGGTAACATTAAGGAC
GAGGTTGACTACCATAAGAACTATCCGACAATTTATCACCTGCGCAAGAAGCTGGCAGACTC
CGACGAGAAGGCAGACCTTAGACTGATTTACCTCGCTCTGGCTCACATCATAAAATTTCGAG
GACACTTCTTGATAGAAGGAGATCTCGACAGCCAGAATACTGATGTTAACGCCCTGTTCCTG
AAATTAGTCGACACCTACAACCTCATGTTTGAGGATGACAAAATCGATACGCAGACTATTGA
CGCAACAGTGATTTTAACTGAGAAGATGAGTAAGTCACGGCGACTTGAGAACTTGATAGCCA
AGATACCTAATCAAAAGAAGAATACCCTCTTCGGAAATCTGATTTCACTCAGTCTTGGCCTG
ACACCTAACTTTAAAGCTAATTTTGAATTGAGCGAGGACGCGAAGCTTCAAATCTCTAAGGA
CTCCTTCGAAGAAGATTTGGATAACCTCCTCGCCCAGATCGGTGACCAATACGCTGACCTGT
TTATAGCAGCGAAGAATTTGTCTGACGCTATCCTCCTGTCTGATATCCTTACTGTGAAGGGC
GTGAATACAAAGGCACCCTTATCCGCCAGTATGGTCCAGCGGTTCAACGAACATCAAGACGA
CCTGAAGTTGCTCAAAAAACTCGTGAAGGTGCAACTGCCCGAGAAATACAAAGAAATTTTCG
ACATTAAAGACAAAAATGGGTACGCTGGGTATATTAACGGTAAGACATCCCAGGAGGACTTT
TACAAATATATCAAGCCTATCTTAAGCAAGCTGAAAGGGGCGGAGTCCCTTATCTCTAAATT
GGAGAGAGAAGACTTTTTGCGGAAGCAGAGAACCTTCGATAATGGATCCATTCCCCACCAGA
TTCACTTGAATGAGCTCAAATCCATCATCCGACGACAGGAGAAGTATTATCCCTTTCTGAAG
GATAAACAGGTGCGGATTGAAAAGATCTTCACCTTTAGAATACCATATTTTGTTGGACCATT
GGCTAACGGGAACTCTTCATTTGCTTGGGTTAAGCGAAGATCTAACGAATCTATTACACCAT
GGAACTTTGAGGAAGTCGTTGAGCAGGAGGCCAGCGCCAAGGTCTTCATAGAGCGGATGACT
AATTTTGATACCTACCTGCCAGAGGAGAAGGTCCTTCCCAAGCACTCTTTGCTCTATGAAAT
GTTCACTGTATACAACGAACTGACTAAAGTAAAGTATCAGGCCGAGGGCATGAGAAAGCCCG
AATTCTTGAGTTCAGAAGAAAAGATTGAGATTGTGTCCAACCTGTTTAAGAAGGAGAGAAAG
GTGACAGTCAAGCAGCTTAAGGAAAATTATTTCAATAAGATAAGATGTCTTGACTCAATCAC
CATCAGTGGGGTTGAAGACAAGTTCAACGCATCACTGGGTACTTACCACGATTTACTTAACA
TTATTAAGAACCAGAAGATTCTGGACGATGAGCAGAACCAGGACTCCCTCGAGGATATTGTG
TTGACTCTGACACTGTTCGAGGACGAAAAAATGATCGCGAAGAGGCTGTCAAAGTATGAATC
CATTTTCGAGCCCAGCATTTTGAAGAAATTAAAAAAGCGCCACTATACTGGTTGGGGCCGTT
TATCCCAGAAGCTCATCAACGGCATCCGTGATAAACAGACCGGAAAGACCATCCTGGACTTC
CTGATCGACGATGGCCAGGCGAATCGAAATTTCATGCAATTGATTAACGATCCCTCTCTGGA
CTTTGCGTCAATAATCAAGGGGGCCCAGGAAAAGACGATAAAGAGCGAGAAGCTCGAAGAGA
CCATCGCTAATCTCGCCGGATCTCCCGCTATCAAGAAAGGCATCTTACAGTCTGTGAAGATT
GTAGATGAAGTGGTGAAAGTGATGGGCTATGAACCTAGCAACATTGTCATAGAAATGGCCAG
GGAAAATCAGTCAACCCACCGAGGCATAAATAACTCTAGGGAACGATTACGAAAGCTGGAGG
AGGTCCACAAGAACATTGGCTCCAAGATCTTGAAAGAGCACGAAATTAGCAATGCCCAACTC
CAGAGTGACCGAGTGTACTTGTATCTGTTGCAGGATGGAAAAGATATGTACACCGGTAAGGA
CCTCGATTTCGATCGGCTCTCTCAGTACGATATTGATcaCATCATACCACAGTCCTTTATTA
AGGACAACAGTATTGATAATATCGTCCTGACATCTCAGGAAAGCAATAGAGGAAAGTCAGAT
AATGTGCCCTACATTGCAATCGTGAATAAGATGAAATCATACTGGCAACACCAGCTGAAATC
TGGGGCTATCAGCCAGCGGAAATTTGATAATTTAACTAAGGTGGAGCGGGGCGGCCTCAGCG
AGTATGATAAGGCAGGTTTTATCAAACGTCAGCTCGTTGAGACACGTCAGATAACAAAGCAC
GTGGCACAAATCCTTAATAATAGATTCAACAACAACGTCGATAACAGTAGCAAGAACAAAAG
ACCTGTCAAGATAATCACATTAAAATCTAAAATGGTGTCTGATTTCCGTAAGGAATTCGGCT
TCTATAAAATTAGGGAGGTAAATGACTATCATCACGCCCACGACGCCTACCTCAACGCCGTT
GTCGGGACAGCCCTGTTGAAAAAATATCCAAAGCTGGAGGCAGAATTCGTGTACGGCGATTA
CAAGCACTATGACTTGGCCTCACTGGTTGTCAAGAGCGACACTAGTCTGGGCAAAGCCACTG
CAAAAATGTTTTTTTATTCTAATATCATGAACTTCTTCAAAAAGGAGGTCAGACTGGCAGAT
GGCACCGTGATCACAAGACCTCAGATAGAGACTAATACGGAAACTGGCGAGATCGTGTGGGA
TAAGGTAAAGGACATTAAAACAATTAGGAAGGTGCTGTCTATACCCCAGATCAACGTGGTTA
AAAAGACTGAAGTCCAAACTGGGGGTTTCTCAAAGGAAAGCATCCTGCCCAAGGGCGATAGC
GATAAGCTTATTCCTAGAAAGAACAATTGGGATCCAAAGAAGTATGGTGGCTTTGATTCTCC
GATCATTGCCTATTCTGTCTTAGTGGTCGCAAAAGTGGCGAAGGGCAAAAGCCAGAAGACAA
AGAGTGTCAAAGAACTTGTCGGAATTACTATCATGGAACAGAACGAGTTCGAAAAGGATCGG
ATTACATTCCTTGAGAAAAAAGGATACCAGGATATTCAGGAATCACTGATCATTAAGCTGCC
CAAGTTCAGCTTGTTTGAGCTTGAAAACGGGAGAAAGCGTCTGCTCGCCAGCGCAAAAGAGC
TCCAGAAGGGAAATGAGCTGTCATTGCCAAACAAGTACATCCAATTTTTGTATCTCGCCTCC
AGATATACTAGCTTTAGCGGCAAGGAGGAAGATAGAGAGAAGCACAGACACTTCGTGGAATC
TCACCTGCACTACTTTGATGAGATTAAAGACATAATTGCCGATTTTTCTCGACGCTATATTC
TGGCAGATGCGAACCTTGAAAAAATTCTCACGCTGTACAATGAGAAAAATCAGTTCTCAATT
GAAGAGCAGGCTACCAACATGCTGAACCTCTTCACCTTCACGGGACTGGGAGCCCCTGCCAC
CCTGAAATTTTTCAACGTGGACATTGATCGGAAGCGATACACTTCCTCCACCGAGATTCTGA
ATAGTACCCTCATTAGACAGAGTATTACCGGACTCTACGAGACAAGGATTGACCTCTCCAAA
ATTGGCGGGGAC
Streptococcus uberis dCas9-p300, protein
SEQ ID NO: 253
MTNGMILGLAIGVASVGVGIIEADSGKVIHASSRIFPSANADNNVERRKERGSRRLLRRKKH
RVKRLQDLFDKYDIVTNFDNLNLNPYELRVKGLNEPLSNEELFASLRNITKHRGISYLDDAE
DDSSGNGTEYAKAIELNQQLLKEKTPGQIQYDRLNQYGQLRGNFDIVDENGEIHHVINVEST
SSYRKEAEQILKKQSETNTSISTDFINDFIQLLTSKRKYYHGPGNPKSRTDYGRYRTDGTDL
DNIFDVLIGKCSFYPEEYRASKTSYTAQEFNFLNDLNNLTLPTETGKLSEQQKIDLVNWAKE
TKILGPKKLLQEIAKRNNCKYEDIRGYRLDKKDNPDMHVFDVYRKMNFDLETISVKDLSVDS
LNQLARILTLNTEREGIEEAIKNLMPNQFTEKQMLELIAFRKSNSSIFGKGWHSLSIKLMKE
LIPELYHTSDEQMTILNRFGKFKLTKLDSKRINYIDENFVTDEIYNPVVAKSVRQAIKIINA
SIKKWGDFDKIVIEMPRDKNEEEERKRIADGQKVNAKEKEQAEKHAAKLENGKEELPSEVFH
GYKELALRIRLWYQQDQKCLYSGKEITISDLIYNRELFEIDAILPLSLSFDDSLSNKVLVYR
WANQEKGQRTPFQALDSMKSAWSYREFKNAILHNSKISRRKRDYFLTEQDISKIEVKQKFIE
RNLVDTRYASRTVLNVLQQSLKNLEKETKVSVVRGQFTSQLRRKWHIDKTRDTYHHHAVDAL
IIAASAKLRYWKKQGDILFENYLINRHVDRVTGEIQSDDSYKEEVFTPPYDGFVQTISNPGF
EDEILFSYQVDSKVNRKISDATIYATRSAKLEKDKKEQTYVLGKIKDIYSQTGFENFLKIYN
KDKSKFLIYQKDPETWEKIIEPILKNYREFDNKGKDIVNPFEKYRNDNGPICKYSRKGNGPE
IKQFKYYDTVYKITSGLDISPRESRNKVILQSLNPWRTDFYFNPKTMKYELMGIRYVDLEFE
KGTGDYLISDNLYKEIKKNEGISELSVFKFTLYKNDLLLIKDTENNEEQIFRFWSRNDLSSK
NRVELKPYDRSRFSGNEILITKMGKAPKQCIKTLTYQNISIYKIKTDILGFKYYLKNEGNKP
LLHFKKTGPKKKRKVASIFKPEELRQALMPTLEALYRQDPESLPFRQPVDPQLLGIPDYFDI
VKSPMDLSTIKRKLDTGQYQEPWQYVDDIWLMENNAWLYNRKTSRVYKYCSKLSEVFEQEID
PVMQSLGYCCGRKLEFSPQTLCCYGKQLCTIPRDATYYSYQNRYHFCEKCFNEIQGESVSLG
DDPSQPQTTINKEQFSKRKNDTLDPELFVECTECGRKMHQICVLHHEIIWPAGFVCDGCLKK
SARTRKENKFSAKRLPSTRLGTFLENRVNDFLRRQNHPESGEVTVRVVHASDKTVEVKPGMK
ARFVDSGEMAESFPYRTKALFAFEEIDGVDLCFFGMHVQEYGSDCPPPNQRRVYISYLDSVH
FFRPKCLRTAVYHEILIGYLEYVKKLGYTTGHIWACPPSEGDDYIFHCHPPDQKIPKPKRLQ
EWYKKMLDKAVSERIVHDYKDIFKQATEDRLTSAKELPYFEGDFWPNVLEESIKELEQEEEE
RKREENTSNESTDVTKGDSKNAKKKNNKKTSKNKSSLSRGNKKKPGMPNVSNDLSQKLYATM
EKHKEVFFVIRLIAGPAANSLPPIVDPDPLIPCDLMDGRDAFLTLARDKHLEFSSLRRAQWS
TMCMLVELHTQSQD
Streptococcus uberis dCas9-p300 nucleotide sequence
(human codon optimized)
SEQ ID NO: 254
ATGACTAATGGGATGATTCTGGGCTTAGCAATCGGAGTCGCGTCTGTAGGGGTAGGAATTAT
CGAGGCCGATAGTGGCAAGGTAATTCACGCAAGCTCACGGATCTTCCCTAGTGCTAATGCCG
ACAATAATGTGGAGCGCCGCAAGTTCCGGGGATCTAGGCGCCTTCTTAGGAGGAAAAAGCAC
AGGGTTAAGCGCTTACAGGATCTGTTTGACAAGTACGATATCGTGACTAACTTCGATAACCT
CAACCTTAACCCCTACGAGCTGCGAGTTAAGGGCTTAAACGAGCCATTGAGCAACGAAGAGC
TCTTCGCATCACTCCGGAACATCACAAAGCACAGGGGCATTTCCTATCTCGATGATGCTGAG
GATGACTCTTCTGGAAACGGGACAGAATACGCCAAAGCGATAGAGCTGAATCAGCAGCTTCT
GAAAGAAAAGACCCCCGGTCAGATCCAGTACGACAGACTCAATCAGTATGGGCAACTGAGAG
GCAATTTCGATATCGTGGATGAAAACGGCGAGATTCACCACGTGATAAACGTTTTTTCAACA
TCAAGTTACAGAAAGGAAGCCGAGCAGATTCTCAAGAAGCAGTCTGAAACGAATACTAGTAT
CAGCACCGACTTTATAAATGATTTCATCCAATTGCTGACCTCTAAGAGGAAATATTACCATG
GTCCTGGTAATCCAAAGAGCCGCACAGATTACGGGCGCTACCGGACGGATGGGACGGATCTC
GATAACATCTTCGATGTTCTGATAGGTAAATGCAGCTTTTACCCAGAGGAGTACCGAGCCAG
CAAGACGAGCTACACTGCCCAAGAGTTCAACTTTCTTAATGACTTGAATAACCTGACCTTAC
CAACCGAGACAGGCAAGTTGAGCGAGCAGCAGAAGATCGACCTGGTGAATTGGGCTAAGGAG
ACAAAGATCCTCGGACCGAAAAAGCTGCTTCAGGAAATTGCCAAGAGGAACAACTGCAAGTA
CGAGGACATTCGCGGCTATCGGCTTGATAAGAAAGATAACCCCGATATGCATGTATTTGATG
TGTATCGGAAGATGAATTTTGACCTGGAGACTATTTCCGTTAAGGATCTGTCAGTCGACTCT
CTGAATCAGCTCGCGCGAATTCTGACACTGAACACCGAGAGGGAGGGGATCGAAGAGGCCAT
CAAAAATCTGATGCCAAACCAGTTCACCGAGAAGCAAATGCTTGAACTCATCGCCTTCCGCA
AGAGTAATTCCTCTATCTTTGGGAAGGGGTGGCACAGTCTGTCAATTAAACTGATGAAAGAG
CTGATACCCGAGCTCTACCACACCAGTGACGAACAAATGACCATACTCAATCGATTTGGTAA
GTTCAAGCTCACGAAGCTCGACTCAAAAAGGACCAATTACATCGATGAAAACTTTGTCACTG
ATGAAATCTATAACCCTGTAGTGGCCAAGAGTGTGAGGCAGGCAATAAAGATCATCAACGCT
TCCATTAAAAAGTGGGGGGACTTTGATAAGATCGTGATTGAGATGCCACGCGACAAGAATGA
GGAGGAGGAAAGGAAACGAATCGCCGATGGCCAGAAGGTGAATGCTAAGGAAAAAGAGCAGG
CCGAGAAGCACGCCGCAAAGCTCTTTAATGGCAAGGAAGAGCTCCCTTCTGAAGTTTTCCAT
GGATATAAGGAGCTGGCTTTGCGAATTAGACTCTGGTATCAGCAAGACCAGAAGTGCCTCTA
TTCTGGCAAGGAGATAACAATTTCAGACCTGATCTACAACAGGGAGCTCTTTGAGATTGACG
CCATCCTTCCGCTGTCTCTTTCTTTTGACGACAGTCTGTCTAACAAGGTCCTGGTTTACAGA
TGGGCAAATCAGGAGAAGGGCCAGAGGACCCCTTTCCAAGCCCTTGATTCCATGAAATCAGC
GTGGTCCTATCGGGAGTTCAAGAATGCAATCCTGCACAATTCTAAAATCAGCCGGAGAAAGC
GTGACTATTTTCTGACAGAACAAGACATTAGTAAGATTGAGGTGAAACAAAAGTTTATTGAG
AGGAACTTGGTGGACACACGGTACGCCAGTAGAACAGTTCTCAACGTGCTGCAGCAGTCCCT
GAAGAATCTGGAGAAGGAGACTAAGGTGTCCGTTGTCCGAGGACAGTTCACGTCCCAGCTGC
GCCGGAAATGGCACATAGATAAGACCAGGGATACTTACCATCACCATGCGGTGGACGCACTG
ATTATCGCGGCCTCCGCTAAGTTGAGATATTGGAAGAAACAGGGCGACATCTTGTTCGAGAA
CTATCTCATCAATCGCCACGTAGATAGAGTAACCGGGGAGATACAATCTGACGATAGCTATA
AGGAGGAGGTGTTCACACCTCCCTACGACGGATTTGTCCAGACTATTAGCAACCCAGGGTTT
GAGGACGAGATCCTTTTCTCCTATCAGGTAGACAGTAAAGTCAACAGAAAGATCTCAGACGC
CACGATATACGCTACGAGGTCTGCGAAGCTCGAGAAGGACAAGAAGGAACAGACGTATGTCT
TGGGTAAGATAAAAGATATCTATTCACAAACTGGTTTTGAGAACTTCCTGAAGATCTATAAT
AAGGACAAGAGTAAGTTCCTGATCTACCAGAAGGACCCTGAGACTTGGGAAAAGATCATTGA
ACCAATTCTCAAAAATTATCGGGAATTCGATAATAAAGGCAAGGATATCGTGAATCCATTTG
AGAAATACAGGAATGATAACGGGCCTATCTGCAAGTACAGTCGGAAAGGCAACGGCCCTGAG
ATCAAACAATTTAAATACTACGACACCGTTTACAAAATTACAAGCGGTCTCGACATCAGCCC
CCGCGAATCAAGAAATAAGGTAATTCTTCAAAGCCTGAATCCGTGGAGAACCGACTTCTACT
TTAACCCTAAGACTATGAAGTACGAACTTATGGGTATCAGATATGTCGACCTGGAGTTCGAG
AAAGGAACAGGGGACTACCTGATTTCTGACAATCTCTATAAAGAGATTAAAAAGAACGAGGG
GATCTCTGAGCTGAGTGTATTCAAGTTCACACTCTACAAGAACGATCTCCTGCTGATCAAGG
ACACTGAGAACAACGAAGAGCAAATTTTTAGGTTTTGGTCTCGGAATGACCTGTCCTCCAAA
AACCGGGTGGAACTGAAGCCCTACGATAGGTCCCGCTTTTCCGGCAATGAGATCCTTATCAC
CAAAATGGGCAAGGCACCTAAGCAATGCATTAAGACTTTAACATACCAAAACATCTCCATTT
ATAAAATCAAAACAGACATCCTGGGATTCAAATACTATCTGAAAAACGAAGGAAATAAGCCA
TTACTGCACTTTAAGAAGACCGGTCCTAAGAAAAAGCGGAAAGTGGctagCattttcaaacc
agaagaactacgacaggcactgatgccaactttggaggcactttaccgtcaggatccagaat
cccttccctttcgtcaacctgtggaccctcagcttttaggaatccctgattactttgatatt
gtgaagagccccatggatctttctaccattaagaggaagttagacactggacagtatcagga
gccctggcagtatgtcgatgatatttggcttatgttcaataatgcctggttatataaccgga
aaacatcacgggtatacaaatactgctccaagctctctgaggtctttgaacaagaaattgac
ccagtgatgcaaagccttggatactgttgtggcagaaagttggagttctctccacagacact
gtgttgctacggcaaacagttgtgcacaatacctcgtgatgccacttattacagttaccaga
acaggtatcatttctgtgagaagtgtttcaatgagatccaaggggagagcgtttctttgggg
gatgacccttcccagcctcaaactacaataaataaagaacaattttccaagagaaaaaatga
cacactggatcctgaactgtttgttgaatgtacagagtgcggaagaaagatgcatcagatct
gtgtccttcaccatgagatcatctggcctgctggattcgtctgtgatggctgtttaaagaaa
agtgcacgaactaggaaagaaaataagttttctgctaaaaggttgccatctaccagacttgg
cacctttctagagaatcgtgtgaatgactttctgaggcgacagaatcaccctgagtcaggag
aggtcactgttagagtagttcatgcttctgacaaaaccgtggaagtaaaaccaggcatgaaa
gcaaggtttgtggacagtggagagatggcagaatcctttccataccgaaccaaagccctctt
tgcctttgaagaaattgatggtgttgacctgtgcttctttggcatgcatgttcaagagtatg
gctctgactgccctccacccaaccagaggagagtatacatatcttacctcgatagtgttcat
ttcttccgtcctaaatgcttgaggactgcagtctatcatgaaatcctaattggatatttaga
atatgtcaagaaattaggttacacaacagggcatatttgggcatgtccaccaagtgagggag
atgattatatcttccattgccatcctcctgaccagaagatacccaagcccaagcgactgcag
gaatggtacaaaaaaatgcttgacaaggctgtatcagagcgtattgtccatgactacaagga
tatttttaaacaagctactgaagatagattaacaagtgcaaaggaattgccttatttcgagg
gtgatttctggcccaatgttctggaagaaagcattaaggaactggaacaggaggaagaagag
agaaaacgagaggaaaacaccagcaatgaaagcacagatgtgaccaagggagacagcaaaaa
tgctaaaaagaagaataataagaaaaccagcaaaaataagagcagcctgagtaggggcaaca
agaagaaacccgggatgcccaatgtatctaacgacctctcacagaaactatatgccaccatg
gagaagcataaagaggtcttctttgtgatccgcctcattgctggccctgctgccaactccct
gcctcccattgttgatcctgatcctctcatcccctgcgatctgatggatggtcgggatgcgt
ttctcacgctggcaagggacaagcacctggagttctcttcactccgaagagcccagtggtcc
accatgtgcatgctggtggagctgcacACGCAGAGCCAGGAC
Streptococcus pyogenes dCas9-p300, protein
SEQ ID NO: 255
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLEKTNRK
VTVKQLKEDYFKKIECFDSVEISGVEDRFNASLGTYHDLLKIIKDKDELDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGDSRADPKKKRKVASIFKPEELRQALMPTLEALYRQDPESLPFRQPVDPQLLGIPDYFDI
VKSPMDLSTIKRKLDTGQYQEPWQYVDDIWLMENNAWLYNRKTSRVYKYCSKLSEVFEQEID
PVMQSLGYCCGRKLEFSPQTLCCYGKQLCTIPRDATYYSYQNRYHFCEKCFNEIQGESVSLG
DDPSQPQTTINKEQFSKRKNDTLDPELFVECTECGRKMHQICVLHHEIIWPAGFVCDGCLKK
SARTRKENKFSAKRLPSTRLGTFLENRVNDFLRRQNHPESGEVTVRVVHASDKTVEVKPGMK
ARFVDSGEMAESFPYRTKALFAFEEIDGVDLCFFGMHVQEYGSDCPPPNQRRVYISYLDSVH
FFRPKCLRTAVYHEILIGYLEYVKKLGYTTGHIWACPPSEGDDYIFHCHPPDQKIPKPKRLQ
EWYKKMLDKAVSERIVHDYKDIFKQATEDRLTSAKELPYFEGDEWPNVLEESIKELEQEEEE
RKREENTSNESTDVTKGDSKNAKKKNNKKTSKNKSSLSRGNKKKPGMPNVSNDLSQKLYATM
EKHKEVFFVIRLIAGPAANSLPPIVDPDPLIPCDLMDGRDAFLTLARDKHLEFSSLRRAQWS
TMCMLVELHTQSQD
Streptococcus pyogenes dCas9-p300 nucleotide sequence
(human codon optimized)
SEQ ID NO: 256
ATGGACAAGAAGTACTCCATTGGGCTCGCCATCGGCACAAACAGCGTCGGCTGGGCCGTCAT
TACGGACGAGTACAAGGTGCCGAGCAAAAAATTCAAAGTTCTGGGCAATACCGATCGCCACA
GCATAAAGAAGAACCTCATTGGCGCCCTCCTGTTCGACTCCGGGGAAACCGCCGAAGCCACG
CGGCTCAAAAGAACAGCACGGCGCAGATATACCCGCAGAAAGAATCGGATCTGCTACCtgca
GGAGATCTTTAGTAATGAGATGGCTAAGGTGGATGACTCTTTCTTCCATAGGCTGGAGGAGT
CCTTTTTGGTGGAGGAGGATAAAAAGCACGAGCGCCACCCAATCTTTGGCAATATCGTGGAC
GAGGTGGCGTACCATGAAAAGTACCCAACCATATATCATCTGAGGAAGAAGCTTGTAGACAG
TACTGATAAGGCTGACTTGCGGTTGATCTATCTCGCGCTGGCGCATATGATCAAATTTCGGG
GACACTTCCTCATCGAGGGGGACCTGAACCCAGACAACAGCGATGTCGACAAACTCTTTATC
CAACTGGTTCAGACTTACAATCAGCTTTTCGAAGAGAACCCGATCAACGCATCCGGAGTTGA
CGCCAAAGCAATCCTGAGCGCTAGGCTGTCCAAATCCCGGCGGCTCGAAAACCTCATCGCAC
AGCTCCCTGGGGAGAAGAAGAACGGCCTGTTTGGTAATCTTATCGCCCTGTCACTCGGGCTG
ACCCCCAACTTTAAATCTAACTTCGACCTGGCCGAAGATGCCAAGCTTCAACTGAGCAAAGA
CACCTACGATGATGATCTCGACAATCTGCTGGCCCAGATCGGCGACCAGTACGCAGACCTTT
TTTTGGCGGCAAAGAACCTGTCAGACGCCATTCTGCTGAGTGATATTCTGCGAGTGAACACG
GAGATCACCAAAGCTCCGCTGAGCGCTAGTATGATCAAGCGCTATGATGAGCACCACCAAGA
CTTGACTTTGCTGAAGGCCCTTGTCAGACAGCAACTGCCTGAGAAGTACAAGGAAATTTTCT
TCGATCAGTCTAAAAATGGCTACGCCGGATACATTGACGGCGGAGCAAGCCAGGAGGAATTT
TACAAATTTATTAAGCCCATCTTGGAAAAAATGGACGGCACCGAGGAGCTGCTGGTAAAGCT
TAACAGAGAAGATCTGTTGCGCAAACAGCGCACTTTCGACAATGGAAGCATCCCCCACCAGA
TTCACCTGGGCGAACTGCACGCTATCCTCAGGCGGCAAGAGGATTTCTACCCCTTTTTGAAA
GATAACAGGGAAAAGATTGAGAAAATCCTCACATTTCGGATACCCTACTATGTAGGCCCCCT
CGCCCGGGGAAATTCCAGATTCGCGTGGATGACTCGCAAATCAGAAGAGACCATCACTCCCT
GGAACTTCGAGGAAGTCGTGGATAAGGGGGCCTCTGCCCAGTCCTTCATCGAAAGGATGACT
AACTTTGATAAAAATCTGCCTAACGAAAAGGTGCTTCCTAAACACTCTCTGCTGTACGAGTA
CTTCACAGTTTATAACGAGCTCACCAAGGTCAAATACGTCACAGAAGGGATGAGAAAGCCAG
CATTCCTGTCTGGAGAGCAGAAGAAAGCTATCGTGGACCTCCTCTTCAAGACGAACCGGAAA
GTTACCGTGAAACAGCTCAAAGAAGACTATTTCAAAAAGATTGAATGTTTCGACTCTGTTGA
AATCAGCGGAGTGGAGGATCGCTTCAACGCATCCCTGGGAACGTATCACGATCTCCTGAAAA
TCATTAAAGACAAGGACTTCCTGGACAATGAGGAGAACGAGGACATTCTTGAGGACATTGTC
CTCACCCTTACGTTGTTTGAAGATAGGGAGATGATTGAAGAACGCTTGAAAACTTACGCTCA
TCTCTTCGACGACAAAGTCATGAAACAGCTCAAGAGGCGCCGATATACAGGATGGGGGCGGC
TGTCAAGAAAACTGATCAATGGgatcCGAGACAAGCAGAGTGGAAAGACAATCCTGGATTTT
CTTAAGTCCGATGGATTTGCCAACCGGAACTTCATGCAGTTGATCCATGATGACTCTCTCAC
CTTTAAGGAGGACATCCAGAAAGCACAAGTTTCTGGCCAGGGGGACAGTCTTCACGAGCACA
TCGCTAATCTTGCAGGTAGCCCAGCTATCAAAAAGGGAATACTGCAGACCGTTAAGGTCGTG
GATGAACTCGTCAAAGTAATGGGAAGGCATAAGCCCGAGAATATCGTTATCGAGATGGCCCG
AGAGAACCAAACTACCCAGAAGGGACAGAAGAACAGTAGGGAAAGGATGAAGAGGATTGAAG
AGGGTATAAAAGAACTGGGGTCCCAAATCCTTAAGGAACACCCAGTTGAAAACACCCAGCTT
CAGAATGAGAAGCTCTACCTGTACTACCTGCAGAACGGCAGGGACATGTACGTGGATCAGGA
ACTGGACATCAATCGGCTCTCCGACTACGACGTGGATGCCATCGTGCCCCAGTCTTTTCTCA
AAGATGATTCTATTGATAATAAAGTGTTGACAAGATCCGATAAAAATAGAGGGAAGAGTGAT
AACGTCCCCTCAGAAGAAGTTGTCAAGAAAATGAAAAATTATTGGCGGCAGCTGCTGAACGC
CAAACTGATCACACAACGGAAGTTCGATAATCTGACTAAGGCTGAACGAGGTGGCCTGTCTG
AGTTGGATAAAGCCGGCTTCATCAAAAGGCAGCTTGTTGAGACACGCCAGATCACCAAgcac
GTGGCCCAAATTCTCGATTCACGCATGAACACCAAGTACGATGAAAATGACAAACTGATTCG
AGAGGTGAAAGTTATTACTCTGAAGTCTAAGCTGGTCTCAGATTTCAGAAAGGACTTTCAGT
TTTATAAGGTGAGAGAGATCAACAATTACCACCATGCGCATGATGCCTACCTGAATGCAGTG
GTAGGCACTGCACTTATCAAAAAATATCCCAAGCTTGAATCTGAATTTGTTTACGGAGACTA
TAAAGTGTACGATGTTAGGAAAATGATCGCAAAGTCTGAGCAGGAAATAGGCAAGGCCACCG
CTAAGTACTTCTTTTACAGCAATATTATGAATTTTTTCAAGACCGAGATTACACTGGCCAAT
GGAGAGATTCGGAAGCGACCACTTATCGAAACAAACGGAGAAACAGGAGAAATCGTGTGGGA
CAAGGGTAGGGATTTCGCGACAGTCCGGAAGGTCCTGTCCATGCCGCAGGTGAACATCGTTA
AAAAGACCGAAGTACAGACCGGAGGCTTCTCCAAGGAAAGTATCCTCCCGAAAAGGAACAGC
GACAAGCTGATCGCACGCAAAAAAGATTGGGACCCCAAGAAATACGGCGGATTCGATTCTCC
TACAGTCGCTTACAGTGTACTGGTTGTGGCCAAAGTGGAGAAAGGGAAGTCTAAAAAACTCA
AAAGCGTCAAGGAACTGCTGGGCATCACAATCATGGAGCGATCAAGCTTCGAAAAAAACCCC
ATCGACTTTCTCGAGGCGAAAGGATATAAAGAGGTCAAAAAAGACCTCATCATTAAGCTTCC
CAAGTACTCTCTCTTTGAGCTTGAAAACGGCCGGAAACGAATGCTCGCTAGTGCGGGCGAGC
TGCAGAAAGGTAACGAGCTGGCACTGCCCTCTAAATACGTTAATTTCTTGTATCTGGCCAGC
CACTATGAAAAGCTCAAAGGGTCTCCCGAAGATAATGAGCAGAAGCAGCTGTTCGTGGAACA
ACACAAACACTACCTTGATGAGATCATCGAGCAAATAAGCGAATTCTCCAAAAGAGTGATCC
TCGCCGACGCTAACCTCGATAAGGTGCTTTCTGCTTACAATAAGCACAGGGATAAGCCCATC
AGGGAGCAGGCAGAAAACATTATCCACTTGTTTACTCTGACCAACTTGGGCGCGCCTGCAGC
CTTCAAGTACTTCGACACCACCATAGACAGAAAGCGGTACACCTCTACAAAGGAGGTCCTGG
ACGCCACACTGATTCATCAGTCAATTACGGGGCTCTATGAAACAAGAATCGACCTCTCTCAG
CTCGGTGGAGACAGCAGGGCTGACCCCAAGAAGAAGAGGAAGGTGGctagCATTTTCAAACC
AGAAGAACTACGACAGGCACTGATGCCAACTTTGGAGGCACTTTACCGTCAGGATCCAGAAT
CCCTTCCCTTTCGTCAACCTGTGGACCCTCAGCTTTTAGGAATCCCTGATTACTTTGATATT
GTGAAGAGCCCCATGGATCTTTCTACCATTAAGAGGAAGTTAGACACTGGACAGTATCAGGA
GCCCTGGCAGTATGTCGATGATATTTGGCTTATGTTCAATAATGCCTGGTTATATAACCGGA
AAACATCACGGGTATACAAATACTGCTCCAAGCTCTCTGAGGTCTTTGAACAAGAAATTGAC
CCAGTGATGCAAAGCCTTGGATACTGTTGTGGCAGAAAGTTGGAGTTCTCTCCACAGACACT
GTGTTGCTACGGCAAACAGTTGTGCACAATACCTCGTGATGCCACTTATTACAGTTACCAGA
ACAGGTATCATTTCTGTGAGAAGTGTTTCAATGAGATCCAAGGGGAGAGCGTTTCTTTGGGG
GATGACCCTTCCCAGCCTCAAACTACAATAAATAAAGAACAATTTTCCAAGAGAAAAAATGA
CACACTGGATCCTGAACTGTTTGTTGAATGTACAGAGTGCGGAAGAAAGATGCATCAGATCT
GTGTCCTTCACCATGAGATCATCTGGCCTGCTGGATTCGTCTGTGATGGCTGTTTAAAGAAA
AGTGCACGAACTAGGAAAGAAAATAAGTTTTCTGCTAAAAGGTTGCCATCTACCAGACTTGG
CACCTTTCTAGAGAATCGTGTGAATGACTTTCTGAGGCGACAGAATCACCCTGAGTCAGGAG
AGGTCACTGTTAGAGTAGTTCATGCTTCTGACAAAACCGTGGAAGTAAAACCAGGCATGAAA
GCAAGGTTTGTGGACAGTGGAGAGATGGCAGAATCCTTTCCATACCGAACCAAAGCCCTCTT
TGCCTTTGAAGAAATTGATGGTGTTGACCTGTGCTTCTTTGGCATGCATGTTCAAGAGTATG
GCTCTGACTGCCCTCCACCCAACCAGAGGAGAGTATACATATCTTACCTCGATAGTGTTCAT
TTCTTCCGTCCTAAATGCTTGAGGACTGCAGTCTATCATGAAATCCTAATTGGATATTTAGA
ATATGTCAAGAAATTAGGTTACACAACAGGGCATATTTGGGCATGTCCACCAAGTGAGGGAG
ATGATTATATCTTCCATTGCCATCCTCCTGACCAGAAGATACCCAAGCCCAAGCGACTGCAG
GAATGGTACAAAAAAATGCTTGACAAGGCTGTATCAGAGCGTATTGTCCATGACTACAAGGA
TATTTTTAAACAAGCTACTGAAGATAGATTAACAAGTGCAAAGGAATTGCCTTATTTCGAGG
GTGATTTCTGGCCCAATGTTCTGGAAGAAAGCATTAAGGAACTGGAACAGGAGGAAGAAGAG
AGAAAACGAGAGGAAAACACCAGCAATGAAAGCACAGATGTGACCAAGGGAGACAGCAAAAA
TGCTAAAAAGAAGAATAATAAGAAAACCAGCAAAAATAAGAGCAGCCTGAGTAGGGGCAACA
AGAAGAAACCCGGGATGCCCAATGTATCTAACGACCTCTCACAGAAACTATATGCCACCATG
GAGAAGCATAAAGAGGTCTTCTTTGTGATCCGCCTCATTGCTGGCCCTGCTGCCAACTCCCT
GCCTCCCATTGTTGATCCTGATCCTCTCATCCCCTGCGATCTGATGGATGGTCGGGATGCGT
TTCTCACGCTGGCAAGGGACAAGCACCTGGAGTTCTCTTCACTCCGAAGAGCCCAGTGGTCC
ACCATGTGCATGCTGGTGGAGCTGCACACGCAGAGCCAGGAC
Streptococcus aureus dCas9-p300 amino acid sequence
SEQ ID NO: 257
MAPKKKRKVGIHGVPAAKRNYILGLAIGITSVGYGIIDYETRDVIDAGVRLFKEANVENNEG
RRSKRGARRLKRRRRHRIQRVKKLLFDYNLLTDHSELSGINPYEARVKGLSQKLSEEEFSAA
LLHLAKRRGVHNVNEVEEDTGNELSTKEQISRNSKALEEKYVAELQLERLKKDGEVRGSINR
FKTSDYVKEAKQLLKVQKAYHQLDQSFIDTYIDLLETRRTYYEGPGEGSPFGWKDIKEWYEM
LMGHCTYFPEELRSVKYAYNADLYNALNDLNNLVITRDENEKLEYYEKFQIIENVFKQKKKP
TLKQIAKEILVNEEDIKGYRVTSTGKPEFTNLKVYHDIKDITARKEIIENAELLDQIAKILT
IYQSSEDIQEELTNLNSELTQEEIEQISNLKGYTGTHNLSLKAINLILDELWHINDNQIAIF
NRLKLVPKKVDLSQQKEIPTTLVDDFILSPVVKRSFIQSIKVINAIIKKYGLPNDIIIELAR
EKNSKDAQKMINEMQKRNRQTNERIEEIIRTTGKENAKYLIEKIKLHDMQEGKCLYSLEAIP
LEDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYFRV
NNLDVKVKSINGGFTSFLRRKWKFKKERNKGYKHHAEDALIIANADFIFKEWKKLDKAKKVM
ENQMFEEKQAESMPEIETEQEYKEIFITPHQIKHIKDFKDYKYSHRVDKKPNRELINDTLYS
TRKDDKGNTLIVNNLNGLYDKDNDKLKKLINKSPEKLLMYHHDPQTYQKLKLIMEQYGDEKN
PLYKYYEETGNYLTKYSKKDNGPVIKKIKYYGNKLNAHLDITDDYPNSRNKVVKLSLKPYRF
DVYLDNGVYKFVTVKNLDVIKKENYYEVNSKCYEEAKKLKKISNQAEFIASFYNNDLIKING
ELYRVIGVNNDLLNRIEVNMIDITYREYLENMNDKRPPRIIKTIASKTQSIKKYSTDILGNL
YEVKSKKHPQIIKKGTGPKKKRKVASIFKPEELRQALMPTLEALYRQDPESLPFRQPVDPQL
LGIPDYFDIVKSPMDLSTIKRKLDTGQYQEPWQYVDDIWLMENNAWLYNRKTSRVYKYCSKL
SEVFEQEIDPVMQSLGYCCGRKLEFSPQTLCCYGKQLCTIPRDATYYSYQNRYHFCEKCENE
IQGESVSLGDDPSQPQTTINKEQFSKRKNDTLDPELFVECTECGRKMHQICVLHHEIIWPAG
FVCDGCLKKSARTRKENKFSAKRLPSTRLGTFLENRVNDFLRRQNHPESGEVTVRVVHASDK
TVEVKPGMKARFVDSGEMAESFPYRTKALFAFEEIDGVDLCFFGMHVQEYGSDCPPPNQRRV
YISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESI
KELEQEEEERKREENTSNESTDVTKGDSKNAKKKNNKKTSKNKSSLSRGNKKKPGMPNVSND
LSQKLYATMEKHKEVFFVIRLIAGPAANSLPPIVDPDPLIPCDLMDGRDAFLTLARDKHLEF
SSLRRAQWSTMCMLVELHTQSQD
Streptococcus aureus dCas9-p300 DNA sequence
SEQ ID NO: 258
ATGGCCCCAAAGAAGAAGCGGAAGGTCGGTATCCACGGAGTCCCAGCAGCCAAGCGGAACTA
CATCCTGGGCCTGGCCATCGGCATCACCAGCGTGGGCTACGGCATCATCGACTACGAGACAC
GGGACGTGATCGATGCCGGCGTGCGGCTGTTCAAAGAGGCCAACGTGGAAAACAACGAGGGC
AGGCGGAGCAAGAGAGGCGCCAGAAGGCTGAAGCGGCGGAGGCGGCATAGAATCCAGAGAGT
GAAGAAGCTGCTGTTCGACTACAACCTGCTGACCGACCACAGCGAGCTGAGCGGCATCAACC
CCTACGAGGCCAGAGTGAAGGGCCTGAGCCAGAAGCTGAGCGAGGAAGAGTTCTCTGCCGCC
CTGCTGCACCTGGCCAAGAGAAGAGGCGTGCACAACGTGAACGAGGTGGAAGAGGACACCGG
CAACGAGCTGTCCACCAAAGAGCAGATCAGCCGGAACAGCAAGGCCCTGGAAGAGAAATACG
TGGCCGAACTGCAGCTGGAACGGCTGAAGAAAGACGGCGAAGTGCGGGGCAGCATCAACAGA
TTCAAGACCAGCGACTACGTGAAAGAAGCCAAACAGCTGCTGAAGGTGCAGAAGGCCTACCA
CCAGCTGGACCAGAGCTTCATCGACACCTACATCGACCTGCTGGAAACCCGGCGGACCTACT
ATGAGGGACCTGGCGAGGGCAGCCCCTTCGGCTGGAAGGACATCAAAGAATGGTACGAGATG
CTGATGGGCCACTGCACCTACTTCCCCGAGGAACTGCGGAGCGTGAAGTACGCCTACAACGC
CGACCTGTACAACGCCCTGAACGACCTGAACAATCTCGTGATCACCAGGGACGAGAACGAGA
AGCTGGAATATTACGAGAAGTTCCAGATCATCGAGAACGTGTTCAAGCAGAAGAAGAAGCCC
ACCCTGAAGCAGATCGCCAAAGAAATCCTCGTGAACGAAGAGGATATTAAGGGCTACAGAGT
GACCAGCACCGGCAAGCCCGAGTTCACCAACCTGAAGGTGTACCACGACATCAAGGACATTA
CCGCCCGGAAAGAGATTATTGAGAACGCCGAGCTGCTGGATCAGATTGCCAAGATCCTGACC
ATCTACCAGAGCAGCGAGGACATCCAGGAAGAACTGACCAATCTGAACTCCGAGCTGACCCA
GGAAGAGATCGAGCAGATCTCTAATCTGAAGGGCTATACCGGCACCCACAACCTGAGCCTGA
AGGCCATCAACCTGATCCTGGACGAGCTGTGGCACACCAACGACAACCAGATCGCTATCTTC
AACCGGCTGAAGCTGGTGCCCAAGAAGGTGGACCTGTCCCAGCAGAAAGAGATCCCCACCAC
CCTGGTGGACGACTTCATCCTGAGCCCCGTCGTGAAGAGAAGCTTCATCCAGAGCATCAAAG
TGATCAACGCCATCATCAAGAAGTACGGCCTGCCCAACGACATCATTATCGAGCTGGCCCGC
GAGAAGAACTCCAAGGACGCCCAGAAAATGATCAACGAGATGCAGAAGCGGAACCGGCAGAC
CAACGAGCGGATCGAGGAAATCATCCGGACCACCGGCAAAGAGAACGCCAAGTACCTGATCG
AGAAGATCAAGCTGCACGACATGCAGGAAGGCAAGTGCCTGTACAGCCTGGAAGCCATCCCT
CTGGAAGATCTGCTGAACAACCCCTTCAACTATGAGGTGGACCACATCATCCCCAGAAGCGT
GTCCTTCGACAACAGCTTCAACAACAAGGTGCTCGTGAAGCAGGAAGAAgcCAGCAAGAAGG
GCAACCGGACCCCATTCCAGTACCTGAGCAGCAGCGACAGCAAGATCAGCTACGAAACCTTC
AAGAAGCACATCCTGAATCTGGCCAAGGGCAAGGGCAGAATCAGCAAGACCAAGAAAGAGTA
TCTGCTGGAAGAACGGGACATCAACAGGTTCTCCGTGCAGAAAGACTTCATCAACCGGAACC
TGGTGGATACCAGATACGCCACCAGAGGCCTGATGAACCTGCTGCGGAGCTACTTCAGAGTG
AACAACCTGGACGTGAAAGTGAAGTCCATCAATGGCGGCTTCACCAGCTTTCTGCGGCGGAA
GTGGAAGTTTAAGAAAGAGCGGAACAAGGGGTACAAGCACCACGCCGAGGACGCCCTGATCA
TTGCCAACGCCGATTTCATCTTCAAAGAGTGGAAGAAACTGGACAAGGCCAAAAAAGTGATG
GAAAACCAGATGTTCGAGGAAAAGCAGGCCGAGAGCATGCCCGAGATCGAAACCGAGCAGGA
GTACAAAGAGATCTTCATCACCCCCCACCAGATCAAGCACATTAAGGACTTCAAGGACTACA
AGTACAGCCACCGGGTGGACAAGAAGCCTAATAGAGAGCTGATTAACGACACCCTGTACTCC
ACCCGGAAGGACGACAAGGGCAACACCCTGATCGTGAACAATCTGAACGGCCTGTACGACAA
GGACAATGACAAGCTGAAAAAGCTGATCAACAAGAGCCCCGAAAAGCTGCTGATGTACCACC
ACGACCCCCAGACCTACCAGAAACTGAAGCTGATTATGGAACAGTACGGCGACGAGAAGAAT
CCCCTGTACAAGTACTACGAGGAAACCGGGAACTACCTGACCAAGTACTCCAAAAAGGACAA
CGGCCCCGTGATCAAGAAGATTAAGTATTACGGCAACAAACTGAACGCCCATCTGGACATCA
CCGACGACTACCCCAACAGCAGAAACAAGGTCGTGAAGCTGTCCCTGAAGCCCTACAGATTC
GACGTGTACCTGGACAATGGCGTGTACAAGTTCGTGACCGTGAAGAATCTGGATGTGATCAA
AAAAGAAAACTACTACGAAGTGAATAGCAAGTGCTATGAGGAAGCTAAGAAGCTGAAGAAGA
TCAGCAACCAGGCCGAGTTTATCGCCTCCTTCTACAACAACGATCTGATCAAGATCAACGGC
GAGCTGTATAGAGTGATCGGCGTGAACAACGACCTGCTGAACCGGATCGAAGTGAACATGAT
CGACATCACCTACCGCGAGTACCTGGAAAACATGAACGACAAGAGGCCCCCCAGGATCATTA
AGACAATCGCCTCCAAGACCCAGAGCATTAAGAAGTACAGCACAGACATTCTGGGCAACCTG
TATGAAGTGAAATCTAAGAAGCACCCTCAGATCATCAAAAAGGGCACCGGTCCTAAGAAAAA
GCGGAAAGTGGctagCattttcaaaccagaagaactacgacaggcactgatgccaactttgg
aggcactttaccgtcaggatccagaatcccttccctttcgtcaacctgtggaccctcagctt
ttaggaatccctgattactttgatattgtgaagagccccatggatctttctaccattaagag
gaagttagacactggacagtatcaggagccctggcagtatgtcgatgatatttggcttatgt
tcaataatgcctggttatataaccggaaaacatcacgggtatacaaatactgctccaagctc
tctgaggtctttgaacaagaaattgacccagtgatgcaaagccttggatactgttgtggcag
aaagttggagttctctccacagacactgtgttgctacggcaaacagttgtgcacaatacctc
gtgatgccacttattacagttaccagaacaggtatcatttctgtgagaagtgtttcaatgag
atccaaggggagagcgtttctttgggggatgacccttcccagcctcaaactacaataaataa
agaacaattttccaagagaaaaaatgacacactggatcctgaactgtttgttgaatgtacag
agtgcggaagaaagatgcatcagatctgtgtccttcaccatgagatcatctggcctgctgga
ttcgtctgtgatggctgtttaaagaaaagtgcacgaactaggaaagaaaataagttttctgc
taaaaggttgccatctaccagacttggcacctttctagagaatcgtgtgaatgactttctga
ggcgacagaatcaccctgagtcaggagaggtcactgttagagtagttcatgcttctgacaaa
accgtggaagtaaaaccaggcatgaaagcaaggtttgtggacagtggagagatggcagaatc
ctttccataccgaaccaaagccctctttgcctttgaagaaattgatggtgttgacctgtgct
tctttggcatgcatgttcaagagtatggctctgactgccctccacccaaccagaggagagta
tacatatcttacctcgatagtgttcatttcttccgtcctaaatgcttgaggactgcagtcta
tcatgaaatcctaattggatatttagaatatgtcaagaaattaggttacacaacagggcata
tttgggcatgtccaccaagtgagggagatgattatatcttccattgccatcctcctgaccag
aagatacccaagcccaagcgactgcaggaatggtacaaaaaaatgcttgacaaggctgtatc
agagcgtattgtccatgactacaaggatatttttaaacaagctactgaagatagattaacaa
gtgcaaaggaattgccttatttcgagggtgatttctggcccaatgttctggaagaaagcatt
aaggaactggaacaggaggaagaagagagaaaacgagaggaaaacaccagcaatgaaagcac
agatgtgaccaagggagacagcaaaaatgctaaaaagaagaataataagaaaaccagcaaaa
ataagagcagcctgagtaggggcaacaagaagaaacccgggatgcccaatgtatctaacgac
ctctcacagaaactatatgccaccatggagaagcataaagaggtcttctttgtgatccgcct
cattgctggccctgctgccaactccctgcctcccattgttgatcctgatcctctcatcccct
gcgatctgatggatggtcgggatgcgtttctcacgctggcaagggacaagcacctggagttc
tcttcactccgaagagcccagtggtccaccatgtgcatgctggtggagctgcacACGCAGAG
CCAGGAC
Streptococcus agalactiae dCas9-p300, protein
SEQ ID NO: 259
MNKPYSIGLAIGTNSVGWSIITDDYKVPAKKMRVLGNTDKEYIKKNLIGALLEDGGNTAADR
RLKRTARRRYTRRRNRILYLQEIFAEEMSKVDDSFFHRLEDSFLVEEDKRGSKYPIFATMQE
EKYYHEKFPTIYHLRKELADKKEKADLRLVYLALAHIIKFRGHFLIEDDREDVRNTDIQKQY
QAFLEIFDTTFENNHLLSQNVDVEAILTDKISKSAKKDRILAQYPNQKSTGIFAEFLKLIVG
NQADFKKHFNLEDKTPLQFAKDSYDEDLENLLGQIGDEFADLESVAKKLYDSVLLSGILTVT
DLSTKAPLSASMIQRYDEHHEDLKHLKQFVKASLPENYREVFADSSKDGYAGYIEGKTNQEA
FYKYLLKLLTKQEGSEYFLEKIKNEDFLRKQRTFDNGSIPHQVHLTELRAIIRRQSEYYPFL
KENQDRIEKILTFRIPYYVGPLAREKSDFAWMTRKTDDSIRPWNFEDLVDKEKSAEAFIHRM
TNNDLYLPEEKVLPKHSLIYEKFTVYNELTKVRFLAEGFKDFQFLNRKQKETIFNSLFKEKR
KVTEKDIISFLNKVDGYEGIAIKGIEKQFNASLSTYHDLKKILGKDFLDNTDNELILEDIVQ
TLTLFEDREMIKKCLDIYKDFFTESQLKKLYRRHYTGWGRLSAKLINGIRNKENQKTILDYL
IDDGSANRNEMQLINDDDLSFKPIIDKARTGSHSDNLKEVVGELAGSPAIKKGILQSLKIVD
ELVKVMGYEPEQIVVEMARENQTTAKGLSRSRQRLTTLRESLANLKSNILEEKKPKYVKDQV
ENHHLSDDRLFLYYLQNGRDMYTKKALDIDNLSQYDIDAIIPQAFIKDDSIDNRVLVSSAKN
RGKSDDVPSIEIVKARKMFWKNLLDAKLMSQRKYDNLTKAERGGLTSDDKARFIQRQLVETR
QITKHVARILDERENNEVDNGKKICKVKIVTLKSNLVSNFRKEFGFYKIREVNDYHHAHDAY
LNAVVAKAILTKYPQLEPEFVYGMYRQKKLSKIVHEDKEEKYSEATRKMFFYSNLMNMFKRV
VRLADGSIVVRPVIETGRYMGKTAWDKKKHFATVRKVLSYPQNNIVKKTEIQTGGFSKESIL
AHGNSDKLIPRKTKDIYLDPKKYGGFDSPIVAYSVLVVADIKKGKAQKLKTVTELLGITIME
RSRFEKNPSAFLESKGYLNIRDDKLMILPKYSLFELENGRRRLLASAGELQKGNELALPTQF
MKFLYLASRYNESKGKPEEIEKKQEFVNQHVSYFDDILQLINDESKRVILADANLEKINKLY
QDNKENIPVDELANNIINLFTFTSLGAPAAFKFFDKIVDRKRYTSTKEVLNSTLIHQSITGL
YETRIDLGKLGEDTGPKKKRKVASIFKPEELRQALMPTLEALYRQDPESLPFRQPVDPQLLG
IPDYFDIVKSPMDLSTIKRKLDTGQYQEPWQYVDDIWLMENNAWLYNRKTSRVYKYCSKLSE
VFEQEIDPVMQSLGYCCGRKLEFSPQTLCCYGKQLCTIPRDATYYSYQNRYHFCEKCFNEIQ
GESVSLGDDPSQPQTTINKEQFSKRKNDTLDPELFVECTECGRKMHQICVLHHEIIWPAGFV
CDGCLKKSARTRKENKFSAKRLPSTRLGTFLENRVNDFLRRQNHPESGEVTVRVVHASDKTV
EVKPGMKARFVDSGEMAESFPYRTKALFAFEEIDGVDLCFFGMHVQEYGSDCPPPNQRRVYI
SYLDSVHFFRPKCLRTAVYHEILIGYLEYVKKLGYTTGHIWACPPSEGDDYIFHCHPPDQKI
PKPKRLQEWYKKMLDKAVSERIVHDYKDIFKQATEDRLTSAKELPYFEGDFWPNVLEESIKE
LEQEEEERKREENTSNESTDVTKGDSKNAKKKNNKKTSKNKSSLSRGNKKKPGMPNVSNDLS
QKLYATMEKHKEVFFVIRLIAGPAANSLPPIVDPDPLIPCDLMDGRDAFLTLARDKHLEFSS
LRRAQWSTMCMLVELHTQSQD
Streptococcus agalactiae dCas9-p300, DNA
SEQ ID NO: 260
ATGAACAAGCCTTATTCAATAGGATTAGCTATAGGGACAAATTCTGTGGGGTGGAGTATAAT
CACCGACGATTACAAGGTGCCTGCAAAGAAGATGCGCGTGCTCGGCAATACAGACAAGGAAT
ATATTAAGAAGAACCTGATCGGGGCCCTCCTTTTTGACGGTGGCAACACAGCAGCTGACCGC
CGCCTCAAGAGGACCGCTCGGAGACGGTATACTCGCCGGCGTAATCGGATCCTGTATTTGCA
GGAAATTTTTGCTGAAGAAATGTCTAAGGTGGATGATTCATTCTTTCACCGGCTCGAAGACT
CCTTTCTGGTGGAGGAAGACAAGAGGGGCTCAAAGTACCCAATCTTCGCCACAATGCAAGAA
GAGAAATACTACCACGAGAAGTTTCCCACAATCTATCATCTCAGGAAAGAGCTGGCCGATAA
AAAAGAGAAGGCCGATTTGCGACTGGTTTACTTGGCCTTGGCACACATCATAAAGTTCCGGG
GACACTTTCTGATTGAAGACGACCGTTTTGACGTCCGCAACACTGATATACAGAAGCAATAC
CAAGCGTTCCTTGAGATCTTTGACACCACATTTGAAAACAACCATCTGCTGAGCCAAAATGT
GGACGTGGAAGCCATTCTGACTGATAAGATCTCTAAATCTGCCAAAAAGGACAGAATCCTTG
CCCAGTACCCCAACCAGAAGTCAACTGGCATTTTCGCCGAGTTTCTGAAGTTGATAGTTGGC
AATCAGGCCGATTTTAAGAAGCACTTCAATTTGGAGGACAAAACGCCTCTCCAATTCGCCAA
GGACTCATATGATGAGGACCTGGAGAATCTGCTTGGCCAAATCGGGGATGAGTTCGCTGATC
TTTTTAGCGTGGCAAAGAAGCTCTATGACTCTGTACTCCTGAGCGGAATCCTGACAGTTACC
GATCTTTCAACAAAGGCACCCCTGAGTGCAAGCATGATTCAACGCTACGACGAGCACCATGA
GGATCTGAAACATCTGAAGCAGTTCGTCAAGGCTTCTCTGCCTGAAAACTATCGGGAGGTCT
TCGCCGACTCATCTAAGGACGGCTACGCCGGATACATCGAGGGAAAGACAAATCAGGAGGCT
TTCTACAAGTACCTGTTGAAGCTGCTTACAAAACAGGAGGGGAGCGAATACTTCCTGGAGAA
GATCAAAAACGAGGACTTCCTGCGTAAACAGAGGACTTTCGATAATGGCTCCATTCCTCACC
AGGTGCATCTCACGGAACTGAGAGCTATCATTAGACGTCAGAGTGAGTATTACCCATTTCTG
AAGGAGAACCAAGACCGAATCGAAAAAATTCTGACGTTCCGGATCCCTTACTATGTCGGACC
TTTAGCTAGGGAAAAAAGTGACTTCGCCTGGATGACCCGAAAGACAGATGATAGTATCAGAC
CATGGAACTTTGAAGACCTGGTGGACAAAGAGAAGAGCGCCGAGGCTTTTATTCACAGGATG
ACCAATAATGATCTCTATCTGCCTGAAGAGAAGGTGCTGCCCAAACACAGTCTCATCTACGA
AAAATTTACAGTCTATAACGAACTGACAAAGGTCCGCTTTCTGGCTGAAGGATTCAAGGACT
TTCAATTTCTGAACCGGAAGCAGAAGGAAACTATCTTTAACTCATTGTTTAAGGAAAAGAGG
AAGGTTACCGAAAAAGACATCATCTCCTTTTTAAACAAGGTAGATGGGTACGAAGGGATTGC
CATTAAAGGCATTGAGAAACAGTTTAACGCCAGCCTTTCAACCTACCATGATCTCAAGAAGA
TCCTCGGAAAAGATTTCCTTGACAATACCGACAACGAACTTATCCTGGAGGATATAGTGCAG
ACACTCACTCTGTTCGAGGACAGGGAAATGATAAAGAAGTGCCTCGACATATATAAAGACTT
CTTTACCGAGAGTCAACTGAAAAAGTTGTATAGAAGGCATTACACCGGTTGGGGCCGACTGA
GTGCAAAACTCATTAACGGCATCCGGAATAAGGAGAATCAAAAGACTATCCTCGATTACCTC
ATCGATGACGGAAGCGCAAACAGAAACTTCATGCAACTCATCAACGATGATGACCTGTCTTT
CAAACCAATTATAGACAAAGCCAGGACTGGGAGCCATAGTGACAATCTGAAGGAAGTGGTGG
GAGAGCTGGCAGGCAGCCCCGCAATTAAGAAGGGGATCCTGCAGAGCCTCAAAATTGTCGAT
GAACTCGTGAAGGTCATGGGCTATGAACCTGAACAGATTGTTGTAGAGATGGCCCGAGAGAA
CCAGACTACTGCGAAGGGACTTAGCCGGAGCAGACAACGACTGACCACTTTGCGAGAGAGTC
TGGCGAACCTGAAGTCTAATATTCTCGAGGAAAAAAAGCCAAAGTACGTGAAGGACCAGGTG
GAGAATCACCACCTGAGCGACGACAGACTCTTTCTGTATTATCTGCAGAACGGCAGAGATAT
GTATACGAAGAAGGCACTGGACATAGACAACCTGAGTCAGTATGACATCGATGCCATTATCC
CTCAGGCCTTCATCAAAGACGATTCAATCGACAATCGCGTACTTGTTAGCAGTGCGAAAAAC
CGGGGAAAGTCTGATGACGTCCCATCCATCGAAATAGTGAAGGCAAGGAAGATGTTCTGGAA
GAATCTGCTGGATGCCAAATTAATGTCACAACGGAAGTACGACAACCTGACAAAGGCAGAAA
GGGGGGGCTTAACAAGCGACGATAAGGCAAGGTTTATCCAGAGGCAGTTGGTCGAGACCAGG
CAAATCACCAAACACGTCGCCCGGATCCTGGATGAACGCTTCAACAATGAAGTCGACAATGG
CAAAAAAATCTGTAAAGTCAAGATAGTGACACTGAAGTCAAATCTGGTGAGCAACTTCCGGA
AAGAATTCGGCTTCTATAAAATTCGCGAAGTGAACGACTATCACCATGCGCACGACGCTTAC
CTGAATGCAGTCGTGGCGAAAGCCATTTTGACCAAGTACCCCCAGCTGGAGCCTGAGTTTGT
GTACGGAATGTACCGACAAAAGAAGCTGAGCAAGATTGTACACGAGGATAAGGAAGAGAAAT
ACTCCGAGGCCACTCGGAAGATGTTCTTCTACTCTAATCTGATGAACATGTTTAAGAGAGTG
GTGAGGTTGGCAGACGGCTCCATTGTTGTAAGGCCAGTGATCGAGACTGGGCGATACATGGG
CAAGACAGCGTGGGACAAGAAGAAGCATTTCGCAACCGTACGGAAAGTCCTGTCCTACCCGC
AGAATAACATTGTGAAGAAGACAGAAATACAAACCGGTGGTTTCTCAAAAGAGTCCATTTTA
GCCCATGGCAACAGTGACAAATTGATTCCACGGAAGACCAAAGATATTTATCTGGACCCTAA
AAAATACGGCGGATTCGACTCACCGATCGTGGCATACAGCGTATTGGTGGTGGCCGATATTA
AGAAGGGTAAAGCCCAGAAACTCAAGACTGTTACCGAGCTCCTGGGTATCACTATAATGGAG
AGAAGCCGGTTTGAGAAGAACCCTAGCGCCTTTTTGGAATCCAAGGGGTATCTGAACATTCG
GGACGATAAGCTGATGATCTTGCCTAAATACAGCCTTTTTGAACTGGAGAATGGACGAAGGC
GCCTGCTTGCCTCAGCGGGGGAACTGCAGAAAGGCAATGAGCTGGCCCTTCCTACCCAGTTC
ATGAAATTTTTGTATCTGGCTAGTAGGTATAACGAGTCAAAAGGCAAGCCAGAGGAGATCGA
AAAGAAGCAGGAATTTGTAAACCAGCATGTGTCATACTTTGATGATATCCTGCAGTTAATCA
ATGACTTCAGTAAACGAGTCATTCTCGCAGACGCCAACTTGGAGAAAATTAATAAGCTGTAC
CAGGACAACAAAGAGAATATACCAGTCGACGAGCTTGCAAATAACATTATTAACCTGTTCAC
TTTTACATCCCTGGGGGCCCCTGCTGCGTTCAAATTTTTCGACAAAATCGTGGATCGAAAGC
GATATACATCCACTAAGGAAGTTCTGAACAGCACTCTCATCCACCAGTCTATCACTGGCCTT
TACGAAACGCGTATTGACTTGGGGAAACTCGGAGAGGACACCGGTCCTAAGAAAAAGCGGAA
AGTGGctagCattttcaaaccagaagaactacgacaggcactgatgccaactttggaggcac
tttaccgtcaggatccagaatcccttccctttcgtcaacctgtggaccctcagcttttagga
atccctgattactttgatattgtgaagagccccatggatctttctaccattaagaggaagtt
agacactggacagtatcaggagccctggcagtatgtcgatgatatttggcttatgttcaata
atgcctggttatataaccggaaaacatcacgggtatacaaatactgctccaagctctctgag
gtctttgaacaagaaattgacccagtgatgcaaagccttggatactgttgtggcagaaagtt
ggagttctctccacagacactgtgttgctacggcaaacagttgtgcacaatacctcgtgatg
ccacttattacagttaccagaacaggtatcatttctgtgagaagtgtttcaatgagatccaa
ggggagagcgtttctttgggggatgacccttcccagcctcaaactacaataaataaagaaca
attttccaagagaaaaaatgacacactggatcctgaactgtttgttgaatgtacagagtgcg
gaagaaagatgcatcagatctgtgtccttcaccatgagatcatctggcctgctggattcgtc
tgtgatggctgtttaaagaaaagtgcacgaactaggaaagaaaataagttttctgctaaaag
gttgccatctaccagacttggcacctttctagagaatcgtgtgaatgactttctgaggcgac
agaatcaccctgagtcaggagaggtcactgttagagtagttcatgcttctgacaaaaccgtg
gaagtaaaaccaggcatgaaagcaaggtttgtggacagtggagagatggcagaatcctttcc
ataccgaaccaaagccctctttgcctttgaagaaattgatggtgttgacctgtgcttctttg
gcatgcatgttcaagagtatggctctgactgccctccacccaaccagaggagagtatacata
tcttacctcgatagtgttcatttcttccgtcctaaatgcttgaggactgcagtctatcatga
aatcctaattggatatttagaatatgtcaagaaattaggttacacaacagggcatatttggg
catgtccaccaagtgagggagatgattatatcttccattgccatcctcctgaccagaagata
cccaagcccaagcgactgcaggaatggtacaaaaaaatgcttgacaaggctgtatcagagcg
tattgtccatgactacaaggatatttttaaacaagctactgaagatagattaacaagtgcaa
aggaattgccttatttcgagggtgatttctggcccaatgttctggaagaaagcattaaggaa
ctggaacaggaggaagaagagagaaaacgagaggaaaacaccagcaatgaaagcacagatgt
gaccaagggagacagcaaaaatgctaaaaagaagaataataagaaaaccagcaaaaataaga
gcagcctgagtaggggcaacaagaagaaacccgggatgcccaatgtatctaacgacctctca
cagaaactatatgccaccatggagaagcataaagaggtcttctttgtgatccgcctcattgc
tggccctgctgccaactccctgcctcccattgttgatcctgatcctctcatcccctgcgatc
tgatggatggtcgggatgcgtttctcacgctggcaagggacaagcacctggagttctcttca
ctccgaagagcccagtggtccaccatgtgcatgctggtggagctgcacACGCAGAGCCAGGA
C
Streptococcus mutans dCas9-p300, protein
SEQ ID NO: 261
MKKPYSIGLAIGTNSVGWAVVTDDYKVPAKKMKVLGNTDKSHIKKNLLGALLFDSGNTAADR
RLKRTARRRYTRRRNRILYLQEIFAEEMSKVDDSFFHRLEDSFLVTEDKRGERHPIFGNLEE
EVKYYENFPTIYHLRQYLADNPEKVDLRLVYLALAHIIKFRGHFLIEGKEDTRNNDVQRLFQ
EFLAVYDNTFENSSLQEQNVQVEEILTDKISKSAKKDRVLKLFPNEKSNGRFAEFLKLIVGN
QADFKKHFELEEKAPLQFSKDTYEEDLEELLGKIGDDYADLFTLAKNLYDAILLSGILTADD
SSTKAPLSASMIQRYNEHQMDLAQLKQFIRQKLSDKYNEVESDVSKDGYAGYIDGKTNQEAF
YKYLKGLLNKIEGSGYFLDKIEREDFLRKQRTFDNGSIPHQIHLQEMRAIIRRQAEFYPFLA
DNQDRIEKILTFRIPYYVGPLARGKSDFAWLSRKSADKITPWNFDEIVDKESSAEAFINRMT
NYDLYLPNQKVLPKHSLLYEKFTVYNELTKVKYKTEQGKTAFFDANMKQEIFDGVFKVYRKV
TKDKLMDFLEKEFDEFRIVDLTGLDKENKVFNASYGTYHDLCKILDKDFLDNSKNEKILEDI
VLTLTLFEDREMIRKRLENYSDLLTKEQVKKLERRHYTGWGRLSAELIHGIRNKESRKTILD
YLIDDGNSNRNEMQLINDDALSFKEEIAKAQVIGETDNLNQVVSDIAGSPAIKKGILQSLKI
VDELVKIMGHQPENIVVEMARENQFTNQGRRNSQQRLKGLTDSIKEFGSQILKEHPVENSQL
QNDRLFLYYLQNGRDMYTGEELDIDYLSQYDIDAIIPQAFIKDNSIDNRVLTSSKENRGKSD
DVPSKDVVRKMKPYWSKLLSAKLITQRKEDNLTKAERGGLTDDDKAGFIKRQLVETRQITKH
VARILDERENTETDENNKKIRQVKIVTLKSNLVSNFRKEFELYKVREINDYHHAHDAYLNAV
IGKALLGVYPQLEPEFVYGDYPHFHGHEENKATAKKFFYSNIMNFFKKDDVRTDKNGEIIWK
KDEHISNIKKVLSYPQVNIVKKVEEQTGGFSKESILPKGNSDKLIPRKTKKFYWDTKKYGGF
DSPIVAYSILVIADIEKGKSKKLKTVKALVGVTIMEKMTFERDPVAFLERKGYRNVQEENII
KLPKYSLFKLENGRKRLLASARELQKGNEIVLPNHLGTLLYHAKNIHKVDEPKHLDYVDKHK
DEFKELLDVVSNFSKKYTLAEGNLEKIKELYAQNNGEDLKELASSFINLLTFTAIGAPATEK
FEDKNIDRKRYTSTTEILNATLIHQSITGLYETRIDLSKLGGDTGPKKKRKVASIFKPEELR
QALMPTLEALYRQDPESLPFRQPVDPQLLGIPDYFDIVKSPMDLSTIKRKLDTGQYQEPWQY
VDDIWLMENNAWLYNRKTSRVYKYCSKLSEVFEQEIDPVMQSLGYCCGRKLEFSPQTLCCYG
KQLCTIPRDATYYSYQNRYHFCEKCFNEIQGESVSLGDDPSQPQTTINKEQFSKRKNDTLDP
ELFVECTECGRKMHQICVLHHEIIWPAGFVCDGCLKKSARTRKENKFSAKRLPSTRLGTFLE
NRVNDFLRRQNHPESGEVTVRVVHASDKTVEVKPGMKARFVDSGEMAESFPYRTKALFAFEE
IDGVDLCFFGMHVQEYGSDCPPPNQRRVYISYLDSVHFFRPKCLRTAVYHEILIGYLEYVKK
LGYTTGHIWACPPSEGDDYIFHCHPPDQKIPKPKRLQEWYKKMLDKAVSERIVHDYKDIFKQ
ATEDRLTSAKELPYFEGDFWPNVLEESIKELEQEEEERKREENTSNESTDVTKGDSKNAKKK
NNKKTSKNKSSLSRGNKKKPGMPNVSNDLSQKLYATMEKHKEVFFVIRLIAGPAANSLPPIV
DPDPLIPCDLMDGRDAFLTLARDKHLEFSSLRRAQWSTMCMLVELHTQSQD
Streptococcus mutans dCas9-p300, DNA
SEQ ID NO: 262
ATGAAGAAGCCTTACTCAATTGGCCTGGCTATTGGCACTAATTCAGTGGGATGGGCCGTCGT
TACCGATGATTACAAGGTACCCGCAAAGAAGATGAAGGTCCTTGGTAATACAGATAAAAGTC
ACATAAAGAAGAATCTTCTCGGAGCTCTTCTGTTCGACAGCGGGAACACAGCTGCCGATAGG
CGACTCAAAAGAACTGCTCGCAGGCGCTATACAAGGCGCAGAAACCGCATTCTGTACCTGCA
GGAGATCTTCGCCGAAGAGATGTCCAAAGTGGATGACAGTTTTTTCCATAGGCTCGAGGATA
GCTTCCTGGTGACCGAGGACAAAAGGGGGGAGAGACATCCCATTTTCGGTAATCTTGAAGAG
GAGGTTAAGTACTACGAGAACTTCCCGACTATATATCATCTGCGGCAGTATCTCGCAGACAA
CCCCGAAAAAGTGGACCTGCGACTTGTGTATCTTGCCCTGGCACATATTATAAAATTCAGAG
GCCACTTTCTGATTGAGGGGAAGTTTGATACCCGGAATAATGATGTGCAGCGCCTGTTTCAG
GAATTCTTGGCTGTCTACGACAATACATTTGAGAATAGTAGTTTGCAGGAGCAGAACGTGCA
AGTGGAAGAGATCCTGACAGACAAGATCTCCAAGAGCGCCAAAAAAGATAGGGTGCTCAAAT
TGTTCCCTAATGAGAAATCCAACGGCAGGTTTGCCGAATTCTTGAAACTGATTGTGGGAAAC
CAGGCTGACTTTAAGAAACATTTCGAGCTTGAAGAAAAGGCCCCTTTGCAGTTTTCCAAAGA
CACCTACGAGGAGGATCTGGAGGAACTGCTGGGAAAGATCGGGGATGACTATGCCGATCTGT
TTACCCTCGCCAAGAACCTGTACGATGCGATTCTCTTGTCCGGTATCCTGACGGCAGACGAC
AGTTCAACTAAAGCTCCGCTCTCTGCCAGCATGATTCAGCGATACAATGAGCATCAGATGGA
TCTGGCCCAGCTCAAGCAGTTCATCCGACAGAAACTCAGCGATAAGTACAACGAGGTGTTTA
GCGACGTGTCCAAAGACGGGTACGCAGGCTACATTGACGGCAAGACCAACCAAGAAGCGTTC
TACAAATACCTGAAAGGGCTGCTCAACAAGATAGAAGGATCAGGTTACTTTCTGGATAAAAT
CGAACGGGAGGATTTTTTGCGCAAGCAGCGAACTTTCGACAATGGGTCCATCCCTCATCAGA
TTCACCTGCAGGAAATGAGAGCTATTATTAGGAGACAGGCTGAATTTTACCCTTTTCTGGCA
GATAACCAGGATCGGATCGAGAAAATCTTAACCTTTCGGATCCCATACTATGTGGGCCCACT
GGCCCGTGGCAAATCCGACTTCGCATGGCTGTCACGGAAGTCCGCCGATAAAATTACGCCGT
GGAACTTTGATGAAATTGTCGATAAGGAATCTTCCGCTGAGGCTTTTATCAATCGCATGACC
AATTACGATCTGTACCTGCCTAATCAGAAGGTGTTACCCAAGCATAGCCTGTTGTATGAAAA
ATTCACTGTCTACAATGAACTCACCAAAGTCAAGTACAAGACAGAACAGGGCAAAACCGCCT
TTTTCGACGCTAATATGAAACAGGAAATTTTTGACGGGGTGTTCAAAGTCTATAGAAAGGTC
ACTAAGGACAAACTGATGGATTTTCTGGAGAAGGAATTTGATGAGTTTCGCATAGTTGATCT
TACTGGTTTGGATAAAGAAAATAAGGTCTTCAATGCAAGCTACGGTACATACCACGACCTTT
GTAAAATTCTCGACAAGGATTTCCTCGACAACTCCAAAAATGAAAAGATTCTTGAGGATATC
GTGTTAACCCTGACCCTGTTTGAAGACAGGGAAATGATCCGGAAGCGGCTGGAGAATTACTC
CGACCTGTTGACTAAAGAGCAGGTGAAAAAGCTCGAGAGGCGCCATTACACCGGATGGGGGA
GACTCAGTGCCGAACTTATCCATGGAATTCGAAACAAGGAGAGCAGGAAGACCATTCTCGAT
TATCTGATTGACGATGGTAATAGCAACAGAAATTTTATGCAGCTGATCAACGATGATGCACT
GTCATTTAAGGAGGAAATTGCAAAAGCCCAGGTTATCGGCGAGACCGACAACCTGAATCAGG
TTGTGAGTGACATCGCAGGGAGCCCCGCTATCAAGAAGGGAATCCTCCAGTCCCTCAAGATT
GTCGACGAGCTCGTCAAGATCATGGGGCATCAGCCAGAGAACATTGTCGTGGAGATGGCCCG
CGAAAACCAATTTACCAACCAAGGGAGGCGGAACAGCCAGCAAAGACTGAAGGGCTTAACAG
ATAGCATTAAAGAGTTCGGATCTCAGATACTTAAAGAACACCCCGTCGAAAACTCCCAGTTG
CAGAATGACCGCCTCTTTCTGTATTATCTGCAAAACGGAAGGGACATGTATACGGGAGAGGA
GCTGGATATAGATTACCTTAGTCAATATGATATCGATGCTATAATCCCCCAAGCCTTTATCA
AGGACAACTCTATAGACAATAGGGTCCTGACCTCTAGCAAAGAGAATAGAGGCAAGTCCGAT
GACGTACCTTCTAAGGATGTCGTGCGCAAGATGAAGCCATACTGGAGCAAGCTGCTGTCTGC
AAAGCTTATAACCCAACGAAAGTTCGATAATCTGACTAAGGCCGAGCGCGGCGGGCTGACAG
ATGACGATAAGGCCGGGTTCATTAAGCGCCAGCTGGTGGAAACAAGACAAATCACTAAACAC
GTCGCTCGAATTCTTGATGAGCGGTTTAACACAGAAACGGACGAAAACAACAAAAAGATCCG
CCAGGTAAAAATTGTAACCCTGAAGAGCAACCTTGTTTCTAATTTCAGAAAGGAATTCGAAC
TTTACAAAGTGCGTGAAATCAACGACTACCATCATGCCCATGACGCTTATCTGAACGCTGTC
ATCGGGAAGGCCCTCCTTGGGGTCTATCCTCAGCTGGAGCCTGAATTTGTGTACGGAGATTA
CCCACACTTTCACGGGCACGAAGAGAACAAGGCAACTGCTAAGAAGTTCTTTTATTCAAATA
TCATGAATTTTTTTAAGAAAGACGACGTCAGAACTGATAAAAACGGTGAGATCATTTGGAAG
AAGGACGAACATATCAGTAATATTAAAAAGGTGCTTAGCTATCCTCAGGTGAACATAGTTAA
AAAAGTAGAGGAGCAGACAGGCGGGTTCTCCAAGGAATCCATACTGCCAAAAGGCAACAGCG
ATAAACTGATACCTCGGAAGACTAAAAAATTCTACTGGGATACCAAGAAGTACGGGGGATTT
GACAGCCCCATTGTCGCCTACTCTATATTGGTTATTGCGGACATCGAAAAGGGCAAATCCAA
AAAGCTTAAAACTGTCAAAGCCCTGGTGGGGGTTACCATCATGGAGAAAATGACCTTTGAAC
GCGATCCCGTAGCATTCCTCGAACGCAAGGGCTACCGCAACGTTCAGGAGGAGAATATCATC
AAGTTGCCCAAATATTCTCTCTTTAAGCTGGAGAACGGCAGAAAGCGCCTGCTCGCATCCGC
AAGGGAGTTACAGAAAGGCAACGAAATTGTACTGCCCAATCACCTCGGAACCCTGCTGTATC
ACGCCAAAAATATCCATAAAGTCGACGAACCTAAGCACTTAGACTATGTCGATAAACACAAG
GACGAATTTAAAGAGCTGCTGGACGTGGTTAGCAATTTCTCAAAGAAATACACGCTGGCGGA
AGGTAATCTGGAGAAAATTAAAGAGTTGTACGCTCAGAATAACGGGGAGGATCTTAAAGAAC
TGGCGTCCTCATTTATCAACCTGCTGACCTTCACCGCCATCGGCGCGCCTGCTACATTTAAA
TTCTTCGATAAGAATATCGATAGAAAGAGATATACTTCCACGACCGAAATTTTGAACGCTAC
CCTGATTCACCAATCTATTACCGGGTTATATGAGACACGAATTGATCTGAGCAAACTGGGGG
GGGATACCGGTCCTAAGAAAAAGCGGAAAGTGGctagCattttcaaaccagaagaactacga
caggcactgatgccaactttggaggcactttaccgtcaggatccagaatcccttccctttcg
tcaacctgtggaccctcagcttttaggaatccctgattactttgatattgtgaagagcccca
tggatctttctaccattaagaggaagttagacactggacagtatcaggagccctggcagtat
gtcgatgatatttggcttatgttcaataatgcctggttatataaccggaaaacatcacgggt
atacaaatactgctccaagctctctgaggtctttgaacaagaaattgacccagtgatgcaaa
gccttggatactgttgtggcagaaagttggagttctctccacagacactgtgttgctacggc
aaacagttgtgcacaatacctcgtgatgccacttattacagttaccagaacaggtatcattt
ctgtgagaagtgtttcaatgagatccaaggggagagcgtttctttgggggatgacccttccc
agcctcaaactacaataaataaagaacaattttccaagagaaaaaatgacacactggatcct
gaactgtttgttgaatgtacagagtgcggaagaaagatgcatcagatctgtgtccttcacca
tgagatcatctggcctgctggattcgtctgtgatggctgtttaaagaaaagtgcacgaacta
ggaaagaaaataagttttctgctaaaaggttgccatctaccagacttggcacctttctagag
aatcgtgtgaatgactttctgaggcgacagaatcaccctgagtcaggagaggtcactgttag
agtagttcatgcttctgacaaaaccgtggaagtaaaaccaggcatgaaagcaaggtttgtgg
acagtggagagatggcagaatcctttccataccgaaccaaagccctctttgcctttgaagaa
attgatggtgttgacctgtgcttctttggcatgcatgttcaagagtatggctctgactgccc
tccacccaaccagaggagagtatacatatcttacctcgatagtgttcatttcttccgtccta
aatgcttgaggactgcagtctatcatgaaatcctaattggatatttagaatatgtcaagaaa
ttaggttacacaacagggcatatttgggcatgtccaccaagtgagggagatgattatatctt
ccattgccatcctcctgaccagaagatacccaagcccaagcgactgcaggaatggtacaaaa
aaatgcttgacaaggctgtatcagagcgtattgtccatgactacaaggatatttttaaacaa
gctactgaagatagattaacaagtgcaaaggaattgccttatttcgagggtgatttctggcc
caatgttctggaagaaagcattaaggaactggaacaggaggaagaagagagaaaacgagagg
aaaacaccagcaatgaaagcacagatgtgaccaagggagacagcaaaaatgctaaaaagaag
aataataagaaaaccagcaaaaataagagcagcctgagtaggggcaacaagaagaaacccgg
gatgcccaatgtatctaacgacctctcacagaaactatatgccaccatggagaagcataaag
aggtcttctttgtgatccgcctcattgctggccctgctgccaactccctgcctcccattgtt
gatcctgatcctctcatcccctgcgatctgatggatggtcgggatgcgtttctcacgctggc
aagggacaagcacctggagttctcttcactccgaagagcccagtggtccaccatgtgcatgc
tggtggagctgcacACGCAGAGCCAGGAC
Streptococcus gallolyticus dCas9-p300, protein
SEQ ID NO: 263
MTNGKILGLAIGIASVGVGIIEAKTGKVVHANSRLFSAANAENNAERRGERGSRRLNRRKKH
RVKRVRDLFEKYEIVTDERNLNLNPYELRVKGLTEQLTNEELFAALRTISKRRGISYLDDAE
DDSTGSTDYAKSIDENRRLLKTKTPGQIQLERLEKYGQLRGNFTVYDENGEAHRLINVESTS
DYEKEARKILETQADYNKKITAEFIDDYVEILTQKRKYYHGPGNEKSRTDYGRFRTDGTTLE
NIFGILIGKCSFYPDEYRASKASYTAQEYNFLNDLNNLKVPTETGKLSTEQKEALVEFAKST
ATLGPAKLLKEIAKILDCKVDEIKGYREDDKGKPDLHTFEPYRKLKENLDSVNIDDLSREVL
DKLADILTLNTEREGIEDAIRHNLPNQFTEGQISEIIKVRKSQSTAFNKGWHSFSAKLMNEL
IPELYATSDEQMTILTRLEKFKVNKKSSKNTKTIDEKEVTDEIYNPVVAKSVRQTIKIINAA
VKKYGDFDKIVIEMPRDKNADDEKKFIDKRNKENKKEKDDALKRAAYLYNGTDKLPDEVFHG
NKQLETKIRLWYQQGERCLYSGKPIPIQELVHNSNNFEIDAILPLSLSFDDSLANKVLVYAW
TNQEKGQKTPYQVIDSMDAAWSFREMKDYVLKQKGLGKKKRDYLLTTENIDKIEVKKKFIER
NLVDTRYASRVVLNSLQSALRELGKDTKISVIRGQFTSQLRRKWKIDKSRETYHHHAVDALI
IAASSQLKLWEKQDNPMFVDYGNNQVVDKQTGEILSVSDDEYKELVFQPPYQGFVNMISSKG
FEDEILFSYQVDSKYNRKVSDATIYSTRKAKIGKDKKEETYVLGKIKDIYSQNGEDTFIKKY
NKDKTQFLMYQKDPLTWENVIEVILRDYPTTKKSEDGKNDVKCNPFEEYRRENGLVCKYSKK
GKGTPIKSLKYYDKKLGNCIDITPEGSKNEVVLQSLNPWRADVYENPETLKYELLGLKYSDL
SFEKGTGKYHISQEKYDVIKEKEGIGKKSEFKFTLYRNDLILIKDTASGEQEIYRFLSRTMP
NVKHYAELKPYDKEKFDNVQELVEALGEADKVGRCIKGLNKSNLSIYKVRTDVLGNKYFVKK
EGDKPKLDFKNNKKTGPKKKRKVASIFKPEELRQALMPTLEALYRQDPESLPFRQPVDPQLL
GIPDYFDIVKSPMDLSTIKRKLDTGQYQEPWQYVDDIWLMENNAWLYNRKTSRVYKYCSKLS
EVFEQEIDPVMQSLGYCCGRKLEFSPQTLCCYGKQLCTIPRDATYYSYQNRYHFCEKCFNEI
QGESVSLGDDPSQPQTTINKEQFSKRKNDTLDPELFVECTECGRKMHQICVLHHEIIWPAGE
VCDGCLKKSARTRKENKFSAKRLPSTRLGTFLENRVNDFLRRQNHPESGEVTVRVVHASDKT
VEVKPGMKARFVDSGEMAESFPYRTKALFAFEEIDGVDLCFFGMHVQEYGSDCPPPNQRRVY
ISYLDSVHFFRPKCLRTAVYHEILIGYLEYVKKLGYTTGHIWACPPSEGDDYIFHCHPPDQK
IPKPKRLQEWYKKMLDKAVSERIVHDYKDIFKQATEDRLTSAKELPYFEGDFWPNVLEESIK
ELEQEEEERKREENTSNESTDVTKGDSKNAKKKNNKKTSKNKSSLSRGNKKKPGMPNVSNDL
SQKLYATMEKHKEVFFVIRLIAGPAANSLPPIVDPDPLIPCDLMDGRDAFLTLARDKHLEFS
SLRRAQWSTMCMLVELHTQSQD
Streptococcus gallolyticus dCas9-p300, DNA
SEQ ID NO: 264
ATGACAAACGGCAAAATTCTGGGTCTGGCCATCGGAATCGCTAGCGTTGGCGTGGGAATCAT
TGAAGCGAAGACAGGTAAAGTCGTCCATGCAAATTCTCGATTGTTCTCCGCAGCTAACGCTG
AAAACAATGCGGAGAGAAGGGGTTTCAGAGGCTCTAGGCGGCTCAACCGGCGCAAGAAGCAC
AGGGTAAAAAGAGTGCGAGATCTCTTTGAGAAATATGAGATCGTGACTGATTTTAGAAACCT
GAATCTGAACCCATATGAGCTGAGAGTGAAAGGACTTACGGAACAGCTCACTAATGAAGAGT
TGTTCGCCGCCCTGCGGACCATCAGCAAACGCCGAGGAATTTCCTACCTTGATGACGCCGAA
GATGACAGTACCGGTAGCACAGATTATGCCAAGAGCATTGATGAGAACAGGAGACTGCTGAA
GACTAAGACACCTGGACAGATACAATTGGAACGGCTCGAGAAGTACGGCCAGCTGAGGGGTA
ACTTCACCGTTTATGACGAAAATGGGGAGGCCCATAGACTGATAAATGTGTTCTCAACTTCT
GACTATGAAAAGGAGGCCCGGAAAATCCTCGAGACTCAAGCCGACTACAACAAGAAGATTAC
AGCCGAGTTTATTGACGATTACGTGGAAATTTTAACCCAGAAAAGGAAGTATTACCACGGGC
CAGGAAATGAAAAGAGCCGCACCGACTATGGGAGATTCAGAACGGATGGAACAACCTTAGAG
AATATCTTTGGAATCCTTATTGGTAAATGCTCTTTCTATCCTGACGAGTATCGCGCCAGCAA
AGCCTCCTATACCGCTCAGGAGTACAACTTCTTGAATGATTTGAACAATTTGAAGGTTCCGA
CGGAGACTGGCAAGCTGAGTACCGAGCAAAAGGAGGCCCTTGTGGAATTCGCCAAGTCTACT
GCAACATTAGGTCCTGCTAAACTTCTGAAGGAGATTGCCAAAATTTTGGACTGCAAAGTCGA
TGAAATCAAGGGGTACCGTGAGGATGATAAAGGGAAACCAGACCTGCACACCTTTGAGCCCT
ATAGAAAGTTGAAATTCAATCTGGACAGCGTCAACATTGACGATTTGAGTCGCGAAGTGCTG
GACAAGCTGGCAGACATTTTGACACTTAACACTGAAAGGGAGGGCATTGAGGATGCCATCAG
GCATAACCTGCCCAACCAATTTACTGAGGGCCAGATCTCCGAAATCATCAAGGTGCGCAAAA
GCCAGAGCACTGCTTTCAACAAGGGGTGGCACAGCTTCTCTGCCAAGCTCATGAACGAATTG
ATTCCCGAGCTCTATGCCACAAGCGACGAACAGATGACTATACTTACTCGGCTGGAGAAATT
TAAGGTCAATAAAAAATCCTCCAAAAACACCAAGACGATTGACGAGAAAGAGGTCACTGATG
AAATCTACAATCCAGTTGTAGCCAAGTCTGTCCGGCAAACGATCAAGATCATTAACGCTGCT
GTGAAGAAATATGGAGACTTTGATAAGATTGTGATTGAAATGCCTCGCGACAAGAATGCGGA
CGATGAGAAGAAGTTTATCGATAAGAGAAACAAAGAAAATAAGAAAGAAAAGGATGATGCCC
TGAAGCGGGCAGCTTACCTTTATAATGGAACCGATAAGCTGCCAGATGAGGTGTTTCACGGA
AACAAGCAACTTGAAACCAAGATTCGCCTGTGGTACCAGCAGGGAGAACGGTGTTTGTACTC
AGGCAAGCCTATCCCAATCCAGGAGTTGGTCCACAACTCCAATAACTTCGAAATCGATGCGA
TTCTGCCCCTGTCCCTGAGTTTTGACGACTCCCTGGCCAACAAGGTGCTTGTGTATGCTTGG
ACCAACCAAGAGAAGGGCCAGAAGACGCCCTACCAGGTGATTGATTCTATGGATGCGGCGTG
GTCCTTTCGCGAGATGAAGGACTATGTGCTCAAGCAAAAAGGCCTCGGCAAAAAGAAACGGG
ATTATCTTTTGACCACCGAGAACATTGACAAGATTGAAGTGAAGAAAAAATTCATCGAGCGC
AACTTGGTCGATACCAGATATGCCTCTAGGGTTGTGCTGAACTCACTGCAGTCTGCTTTGAG
AGAGCTGGGTAAAGACACTAAAATTAGTGTAATCAGGGGCCAGTTCACAAGTCAGCTTAGGC
GGAAATGGAAGATCGACAAGTCACGCGAGACATATCATCATCACGCAGTCGACGCACTGATA
ATTGCAGCTTCAAGTCAGCTCAAGTIGTGGGAGAAACAGGATAACCCTATGTTTGTCGACTA
TGGAAACAATCAGGTCGTCGATAAGCAGACCGGGGAAATTTTAAGTGTGTCCGATGACGAGT
ATAAGGAGCTTGTCTTTCAGCCACCGTACCAGGGCTTTGTCAACATGATTAGTAGCAAGGGT
TTTGAGGACGAAATTTTGTTCAGCTACCAGGTCGATTCCAAATACAATAGAAAAGTATCCGA
CGCAACCATATATTCTACTCGCAAGGCCAAGATTGGCAAAGATAAGAAGGAAGAGACCTATG
TATTGGGGAAGATCAAAGACATTTACTCACAAAATGGATTCGACACCTTCATTAAGAAGTAC
AACAAAGATAAGACACAGTTTTTGATGTACCAGAAAGATCCACTGACATGGGAAAACGTGAT
CGAAGTTATACTGCGTGACTACCCCACGACTAAAAAGAGTGAGGACGGAAAAAACGACGTGA
AGTGCAACCCGTTTGAAGAATACCGGAGAGAAAACGGTCTGGTGTGTAAGTACTCTAAGAAA
GGAAAGGGGACCCCTATTAAATCCCTCAAATACTACGACAAAAAACTCGGGAACTGCATCGA
TATCACCCCGGAAGGTTCCAAAAATGAAGTCGTGCTTCAATCCTTGAATCCGTGGAGGGCAG
ATGTGTACTTTAACCCAGAAACCTTGAAGTATGAATTACTGGGACTTAAATACAGTGATCTC
TCATTTGAAAAGGGCACTGGAAAATACCATATCTCTCAGGAGAAGTACGACGTCATTAAGGA
AAAAGAAGGGATCGGGAAAAAATCCGAGTTCAAGTTCACATTGTATAGGAACGACCTGATCC
TTATTAAAGACACAGCCAGCGGTGAGCAGGAGATTTACCGATTTCTGTCTAGAACCATGCCT
AACGTCAAGCACTATGCGGAGCTGAAGCCCTATGACAAAGAAAAATTTGATAACGTCCAGGA
ACTCGTCGAGGCGCTGGGCGAAGCCGACAAGGTAGGCCGCTGTATAAAGGGGCTGAACAAAA
GCAACCTCAGCATCTATAAAGTTAGGACAGATGTGCTCGGGAACAAATACTTCGTTAAGAAG
GAAGGGGACAAGCCCAAGCTGGATTTTAAGAACAATAAAAAGACCGGTCCTAAGAAAAAGCG
GAAAGTGGctagCattttcaaaccagaagaactacgacaggcactgatgccaactttggagg
cactttaccgtcaggatccagaatcccttccctttcgtcaacctgtggaccctcagctttta
ggaatccctgattactttgatattgtgaagagccccatggatctttctaccattaagaggaa
gttagacactggacagtatcaggagccctggcagtatgtcgatgatatttggcttatgttca
ataatgcctggttatataaccggaaaacatcacgggtatacaaatactgctccaagctctct
gaggtctttgaacaagaaattgacccagtgatgcaaagccttggatactgttgtggcagaaa
gttggagttctctccacagacactgtgttgctacggcaaacagttgtgcacaatacctcgtg
atgccacttattacagttaccagaacaggtatcatttctgtgagaagtgtttcaatgagatc
caaggggagagcgtttctttgggggatgacccttcccagcctcaaactacaataaataaaga
acaattttccaagagaaaaaatgacacactggatcctgaactgtttgttgaatgtacagagt
gcggaagaaagatgcatcagatctgtgtccttcaccatgagatcatctggcctgctggattc
gtctgtgatggctgtttaaagaaaagtgcacgaactaggaaagaaaataagttttctgctaa
aaggttgccatctaccagacttggcacctttctagagaatcgtgtgaatgactttctgaggc
gacagaatcaccctgagtcaggagaggtcactgttagagtagttcatgcttctgacaaaacc
gtggaagtaaaaccaggcatgaaagcaaggtttgtggacagtggagagatggcagaatcctt
tccataccgaaccaaagccctctttgcctttgaagaaattgatggtgttgacctgtgcttct
ttggcatgcatgttcaagagtatggctctgactgccctccacccaaccagaggagagtatac
atatcttacctcgatagtgttcatttcttccgtcctaaatgcttgaggactgcagtctatca
tgaaatcctaattggatatttagaatatgtcaagaaattaggttacacaacagggcatattt
gggcatgtccaccaagtgagggagatgattatatcttccattgccatcctcctgaccagaag
atacccaagcccaagcgactgcaggaatggtacaaaaaaatgcttgacaaggctgtatcaga
gcgtattgtccatgactacaaggatatttttaaacaagctactgaagatagattaacaagtg
caaaggaattgccttatttcgagggtgatttctggcccaatgttctggaagaaagcattaag
gaactggaacaggaggaagaagagagaaaacgagaggaaaacaccagcaatgaaagcacaga
tgtgaccaagggagacagcaaaaatgctaaaaagaagaataataagaaaaccagcaaaaata
agagcagcctgagtaggggcaacaagaagaaacccgggatgcccaatgtatctaacgacctc
tcacagaaactatatgccaccatggagaagcataaagaggtcttctttgtgatccgcctcat
tgctggccctgctgccaactccctgcctcccattgttgatcctgatcctctcatcccctgcg
atctgatggatggtcgggatgcgtttctcacgctggcaagggacaagcacctggagttctct
tcactccgaagagcccagtggtccaccatgtgcatgctggtggagctgcacACGCAGAGCCA
GGAC
Streptococcus iniae dCas9-p300 amino acid sequence
SEQ ID NO: 265
MRKPYSIGLAIGTNSVGWAVITDDYKVPSKKMRIQGTTDRTSIKKNLIGALLEDNGETAEAT
RLKRTTRRRYTRRKYRIKELQKIFSSEMNELDIAFFPRLSESFLVSDDKEFENHPIFGNLKD
EITYHNDYPTIYHLRQTLADRDQKADLRLIYLALAHIIKERGHFLIEGNLDSENTDVHVLFL
NLVNIYNNLFEEDIVETASIDAEKILTSKTSKSRRLENLIAEIPNQKRNMLFGNLVSLALGL
TPNFKTNFELLEDAKLQISKDSYEEDLDNLLAQIGDQYADLFIAAKKLSDAILLSDIITVKG
ASTKAPLSASMVQRYEEHQQDLALLKNLVKKQIPEKYKEIFDNKEKNGYAGYIDGKTSQEEF
YKYIKPILLKLNGTEKLISKLEREDELRKQRTFDNGSIPHQIHLNELKAIIRRQEKFYPFLK
ENQKKIEKLFTFKIPYYVGPLANGQSSFAWLKRQSNESITPWNFEEVVDQEASARAFIERMT
NFDTYLPEEKVLPKHSPLYEMFMVYNELTKVKYQTEGMKRPVFLSSEDKEEIVNLLFKKDRK
VTVKQLKEEYESKMKCFHTVTILGVEDRFNASLGTYHDLLKIFKDKAFLDDEANQDILEEIV
WTLTLFEDQAMIERRLVKYADVFEKSVLKKLKKRHYTGWGRLSQKLINGIKDKQTGKTILGF
LKDDGVANRNFMQLINDSSLDFAKIIKHEQEKTIKNESLEETIANLAGSPAIKKGILQSIKI
VDEIVKIMGQNPDNIVIEMARENQSTMQGIKNSRQRLRKLEEVHKNTGSKILKEYNVSNTQL
QSDRLYLYLLQDGKDMYTGKELDYDNLSQYDIDAIIPQSFIKDNSIDNIVLTTQASNRGKSD
NVPNIEIVNKMKSFWYKQLKNGAISQRKFDHLTKAERGALSDEDKAGFIKRQLVETRQITKH
VAQILDSRFNSNLTEDSKSNRNVKIITLKSKMVSDFRKDFGFYKLREVNDYHHAQDAYLNAV
VGTALLKKYPKLEAEFVYGDYKHYDLAKLMIQPDSSLGKATTRMFFYSNLMNFFKKEIKLAD
DTIFTRPQIEVNTETGEIVWDKVKDMQTIRKVMSYPQVNIVMKTEVQTGGFSKESILPKGNS
DKLIARKKSWDPKKYGGFDSPIIAYSVLVVAKIAKGKTQKLKTIKELVGIKIMEQDEFEKDP
IAFLEKKGYQDIQTSSIIKLPKYSLFELENGRKRLLASAKELQKGNELALPNKYVKFLYLAS
HYTKFTGKEEDREKKRSYVESHLYYFDEIMQIIVEYSNRYILADSNLIKIQNLYKEKDNESI
EEQAINMLNLFTFTDLGAPAAFKFENGDIDRKRYSSTNEIINSTLIYQSPTGLYETRIDLSK
LGGKTGPKKKRKVASIFKPEELRQALMPTLEALYRQDPESLPFRQPVDPQLLGIPDYFDIVK
SPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDD
PSQPQTTINKEQFSKRKNDTLDPELFVECTECGRKMHQICVLHHEIIWPAGFVCDGCLKKSA
RTRKENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSVHFF
RPKCLRTAVYHEILIGYLEYVKKLGYTTGHIWACPPSEGDDYIFHCHPPDQKIPKPKRLQEW
YKKMLDKAVSERIVHDYKDIFKQATEDRLTSAKELPYFEGDFWPNVLEESIKELEQEEEERK
REENTSNESTDVTKGDSKNAKKKNNKKTSKNKSSLSRGNKKKPGMPNVSNDLSQKLYATMEK
HKEVFFVIRLIAGPAANSLPPIVDPDPLIPCDLMDGRDAFLTLARDKHLEFSSLRRAQWSTM
CMLVELHTQSQD
Streptococcus iniae dCas9-p300, DNA
SEQ ID NO: 266
ATGCGCAAACCTTACTCAATTGGCCTGGCAATCGGGACTAATTCTGTTGGCTGGGCTGTGAT
TACTGATGATTACAAGGTGCCAAGTAAGAAAATGAGGATTCAGGGCACGACTGATCGGACCA
GCATTAAGAAGAATCTCATTGGGGCCCTCCTGTTCGATAATGGCGAGACTGCCGAGGCCACT
CGATTAAAGAGAACAACAAGGAGGAGGTACACCAGACGGAAGTACCGAATAAAGGAACTGCA
AAAGATCTTCAGCAGCGAAATGAATGAGCTCGACATTGCTTTTTTCCCTAGACTGTCTGAGA
GTTTTCTTGTGAGTGACGACAAAGAATTCGAGAATCATCCGATTTTTGGAAACCTTAAAGAT
GAGATAACTTATCATAACGATTACCCTACTATTTATCACTTGCGACAGACACTTGCAGACCG
TGACCAGAAGGCCGATCTTAGGCTCATTTATCTCGCTCTGGCCCACATTATTAAATTTCGGG
GGCACTTTTTGATCGAAGGCAATCTGGACAGTGAGAACACGGACGTACACGTGCTGTTTCTG
AACCTGGTGAACATATATAATAACCTGTTCGAGGAAGATATAGTTGAAACCGCATCCATAGA
CGCTGAGAAGATTCTTACCTCAAAAACTTCCAAATCCAGGCGGCTCGAGAATCTTATAGCTG
AGATTCCTAACCAGAAGCGGAACATGTTGTTTGGCAACCTCGTGTCTCTGGCTCTCGGCCTG
ACACCAAATTTTAAAACCAATTTTGAGCTGCTGGAGGATGCAAAGTTACAGATCTCCAAGGA
TTCATATGAAGAAGACCTCGACAACTTGTTGGCACAGATTGGGGATCAGTACGCAGATCTCT
TTATCGCCGCTAAAAAGCTTTCTGACGCAATATTACTGTCTGACATCATCACCGTGAAGGGC
GCCTCCACTAAAGCGCCTCTTTCAGCATCCATGGTGCAGAGATATGAAGAGCATCAACAGGA
CCTCGCTCTCCTGAAGAATCTCGTGAAAAAACAGATTCCTGAGAAGTATAAGGAAATCTTCG
ATAACAAGGAGAAGAATGGCTATGCAGGTTATATCGATGGCAAGACCTCCCAGGAGGAATTT
TACAAGTACATCAAGCCCATACTTCTTAAGCTCAACGGCACAGAGAAGTTGATCAGCAAACT
TGAGCGGGAGGACTTCCTGAGAAAGCAACGAACATTCGACAACGGATCTATTCCTCACCAGA
TTCACCTGAATGAGCTCAAGGCAATCATCCGGAGGCAGGAGAAGTTTTATCCCTTTCTGAAG
GAAAATCAGAAGAAAATCGAAAAGCTTTTCACATTTAAAATTCCCTATTACGTCGGGCCACT
CGCCAATGGCCAGAGTAGCTTCGCCTGGCTGAAGAGACAGTCCAACGAGTCTATCACCCCCT
GGAACTTCGAGGAAGTGGTGGATCAAGAGGCCTCAGCGCGCGCCTTCATAGAGAGGATGACT
AACTTCGATACCTATTTACCCGAGGAGAAGGTTCTGCCAAAGCACAGCCCACTCTACGAAAT
GTTTATGGTCTATAATGAGCTCACCAAGGTTAAGTATCAGACCGAGGGGATGAAGAGGCCCG
TCTTTCTCTCTTCCGAAGACAAAGAAGAAATAGTGAATCTCCTGTTCAAAAAAGACCGGAAG
GTCACTGTCAAGCAGCTGAAGGAGGAATATTTCTCCAAAATGAAATGCTTCCACACCGTGAC
AATCTTGGGCGTGGAGGATCGGTTTAATGCTTCTCTGGGCACGTACCATGACCTGCTCAAAA
TTTTTAAAGATAAAGCCTTCTTAGACGATGAGGCCAATCAAGATATCTTGGAAGAGATCGTA
TGGACTTTAACGCTTTTTGAGGATCAAGCCATGATTGAAAGAAGGCTGGTGAAGTACGCGGA
CGTGTTCGAAAAATCCGTCCTTAAAAAGTTAAAGAAACGCCATTACACGGGCTGGGGACGTC
TTTCCCAGAAGCTTATTAATGGGATCAAAGACAAACAAACTGGGAAGACAATTCTCGGCTTT
CTGAAAGACGACGGTGTAGCCAACCGAAATTTTATGCAGTTAATTAACGACAGCTCCCTGGA
CTTCGCAAAGATTATCAAGCATGAACAGGAAAAAACCATCAAGAACGAGTCATTGGAGGAAA
CGATTGCGAACCTGGCAGGCAGCCCCGCCATTAAGAAAGGCATTCTTCAGTCTATTAAAATT
GTCGATGAAATCGTTAAGATTATGGGACAGAACCCAGACAATATTGTTATTGAGATGGCACG
CGAGAACCAATCCACGATGCAAGGAATCAAAAACTCCCGACAGCGTCTGCGCAAGCTCGAGG
AGGTGCATAAGAACACCGGGTCCAAGATTTTGAAAGAATACAACGTGAGTAATACGCAGCTT
CAGAGCGATAGGCTCTATTTATACCTGCTGCAGGACGGAAAGGATATGTACACCGGCAAGGA
GTTGGACTACGACAATCTTAGTCAATATGATATTGATGCGATCATCCCTCAGTCTTTCATAA
AAGATAACTCTATCGACAACATAGTGCTGACTACACAAGCTAGTAATAGGGGCAAGTCAGAC
AACGTGCCCAACATAGAGATTGTGAACAAAATGAAGTCTTTTTGGTATAAACAGCTCAAAAA
TGGGGCAATTAGCCAGCGCAAATTCGACCATTTAACCAAGGCCGAGCGTGGCGCACTGAGCG
ATTTCGATAAGGCAGGCTTTATCAAGCGCCAGCTCGTCGAGACACGGCAGATAACCAAACAT
GTGGCTCAAATCCTGGACAGTCGGTTCAATTCCAATCTTACGGAGGACTCTAAATCTAACAG
AAACGTTAAGATAATAACTCTCAAGTCAAAAATGGTGAGTGACTTCCGAAAGGACTTTGGCT
TTTACAAGCTGAGAGAAGTAAATGATTATCACCACGCCCAGGACGCATATCTCAATGCCGTC
GTCGGTACTGCCTTACTTAAGAAGTACCCTAAACTGGAAGCAGAGTTCGTGTATGGGGATTA
CAAGCACTACGATCTCGCTAAGTTAATGATTCAACCGGACAGTAGCCTTGGAAAAGCCACAA
CCAGAATGTTCTTCTATTCTAACCTCATGAATTTCTTCAAAAAAGAAATCAAACTGGCCGAT
GATACTATATTTACGAGGCCCCAGATTGAAGTGAACACCGAAACTGGGGAGATTGTCTGGGA
TAAGGTAAAGGACATGCAGACCATCAGGAAAGTGATGTCCTATCCACAAGTCAACATAGTGA
TGAAAACCGAAGTCCAGACTGGGGGGTTTTCTAAGGAGAGTATCCTGCCTAAGGGAAACTCA
GACAAACTGATCGCCCGCAAGAAATCCTGGGACCCTAAGAAATACGGTGGTTTCGATAGCCC
TATCATTGCATATTCAGTCCTGGTCGTCGCTAAGATAGCCAAAGGCAAAACCCAGAAACTCA
AGACTATTAAAGAGTTGGTCGGTATCAAAATCATGGAGCAGGACGAATTCGAAAAGGATCCA
ATTGCGTTTCTCGAAAAGAAGGGCTATCAGGACATACAGACCTCTTCCATCATCAAGCTGCC
GAAGTACTCTCTCTTTGAGCTTGAGAATGGACGCAAGAGACTGCTGGCTAGCGCCAAAGAAC
TGCAGAAGGGCAACGAACTGGCCCTCCCTAACAAATACGTAAAGTTCTTGTATTTAGCATCT
CATTACACAAAATTCACAGGTAAGGAGGAAGATCGAGAAAAAAAGCGCTCCTATGTAGAGTC
ACACCTGTATTACTTTGACGAGATTATGCAGATTATCGTTGAGTATTCTAACCGGTACATTC
TCGCCGACAGCAATCTGATTAAAATTCAGAACTTGTACAAAGAGAAGGATAACTTTAGTATC
GAGGAGCAAGCCATTAATATGCTCAATCTCTTCACTTTTACAGATCTCGGCGCGCCAGCCGC
TTTCAAGTTCTTTAACGGAGATATAGATCGGAAGCGGTACAGCTCTACCAACGAGATCATTA
ATTCTACTCTGATTTACCAGAGTCCCACAGGGTTATACGAGACCAGGATCGACCTCAGTAAG
CTGGGGGGCAAAACCGGTCCTAAGAAAAAGCGGAAAGTGGctagCattttcaaaccagaaga
actacgacaggcactgatgccaactttggaggcactttaccgtcaggatccagaatcccttc
cctttcgtcaacctgtggaccctcagcttttaggaatccctgattactttgatattgtgaag
agccccatggatctttctaccattaagaggaagttagacactggacagtatcaggagccctg
gcagtatgtcgatgatatttggcttatgttcaataatgcctggttatataaccggaaaacat
cacgggtatacaaatactgctccaagctctctgaggtctttgaacaagaaattgacccagtg
atgcaaagccttggatactgttgtggcagaaagttggagttctctccacagacactgtgttg
ctacggcaaacagttgtgcacaatacctcgtgatgccacttattacagttaccagaacaggt
atcatttctgtgagaagtgtttcaatgagatccaaggggagagcgtttctttgggggatgac
ccttcccagcctcaaactacaataaataaagaacaattttccaagagaaaaaatgacacact
ggatcctgaactgtttgttgaatgtacagagtgcggaagaaagatgcatcagatctgtgtcc
ttcaccatgagatcatctggcctgctggattcgtctgtgatggctgtttaaagaaaagtgca
cgaactaggaaagaaaataagttttctgctaaaaggttgccatctaccagacttggcacctt
tctagagaatcgtgtgaatgactttctgaggcgacagaatcaccctgagtcaggagaggtca
ctgttagagtagttcatgcttctgacaaaaccgtggaagtaaaaccaggcatgaaagcaagg
tttgtggacagtggagagatggcagaatcctttccataccgaaccaaagccctctttgcctt
tgaagaaattgatggtgttgacctgtgcttctttggcatgcatgttcaagagtatggctctg
actgccctccacccaaccagaggagagtatacatatcttacctcgatagtgttcatttcttc
cgtcctaaatgcttgaggactgcagtctatcatgaaatcctaattggatatttagaatatgt
caagaaattaggttacacaacagggcatatttgggcatgtccaccaagtgagggagatgatt
atatcttccattgccatcctcctgaccagaagatacccaagcccaagcgactgcaggaatgg
tacaaaaaaatgcttgacaaggctgtatcagagcgtattgtccatgactacaaggatatttt
taaacaagctactgaagatagattaacaagtgcaaaggaattgccttatttcgagggtgatt
tctggcccaatgttctggaagaaagcattaaggaactggaacaggaggaagaagagagaaaa
cgagaggaaaacaccagcaatgaaagcacagatgtgaccaagggagacagcaaaaatgctaa
aaagaagaataataagaaaaccagcaaaaataagagcagcctgagtaggggcaacaagaaga
aacccgggatgcccaatgtatctaacgacctctcacagaaactatatgccaccatggagaag
cataaagaggtcttctttgtgatccgcctcattgctggccctgctgccaactccctgcctcc
cattgttgatcctgatcctctcatcccctgcgatctgatggatggtcgggatgcgtttctca
cgctggcaagggacaagcacctggagttctcttcactccgaagagcccagtggtccaccatg
tgcatgctggtggagctgcacACGCAGAGCCAGGAC
Streptococcus lutetiensis dCas9-p300 amino acid sequence
SEQ ID NO: 267
MSNGKILGLAIGVASVGVGIIDAKTGNVIHANSRLFSAANAENNAERRGERGARRLTRRKKH
RVKRVRDLFEKYDISTDERNLNLNPYELRVKGLTEQLTNEELFAALRTIAKRRGISYLDDAE
DDSTGSSDYAKSIDENRRLLKTKTPGQIQLERLEKYGQLRGNFTVYDENGEAHRLINVESTS
DYKNEARKILETQSNYNKQITDEFIEDYIEILTQKRKYYHGPGNEKSRTDYGRFRTDGTTLE
NIFGILIGKCSFYPEEYRASKASYTAQEFNFLNDLNNLKVPTETGKLSTEQKEYLVDFAKKS
KALGASKLLKEIAKIVDCSVDDIKGYRVDNKDKPDLHTFEPYRKLKENLSSIDIDELSRETL
DKLADILTLNTEREGIEDTIKRNLPSQFTEEQISEIVQIRKNQSSAFNKGWHSFSAKLMNEL
IPELYVTSEEQMTILTRLEKFKVNKKSSKNTKTIDEKEITDEIYNPVVAKSVRQTIKIINAA
VKKYGDFDKIVIEMPRDKNAEDEKKFIDKKEKENKKEKDDSLKRAAFLYNGTDNLPDGVFHG
NKELKTKIRLWYQQGERCLYSGKLISIHDLVHNSNKFEIDAILPLSLSFDDSLANKVLVYAW
TNQEKGQKTPYQVIDSMDAAWSFREMKDYVLKQKRLGKKKREYLLTTENIDKIEVKKKFIER
NLVDTRYASRVVLNSLQTALKELGKDTKVSVVRGQFTSQLRRKWNIDKSRETYHHHAVDALI
IAASSQLKLWQKQENPMFESYGENQVVNKETGEILSISDDKYKELVFQPPYQGFVNTISSKG
FEDEILFSYQVDSKENRKVSDATIYSTRKAKLGKDKKDETYVLGKIKDIYSQDGFDTFIKRY
KKDKTQFLMYQKDPLTWENVIEVILRDYPSEKLSEDGKKTVKCNPFEEYRRENGLICKYSKK
GNGTPIKSLKYYDKKLGNCIDITPEKSKNRVVLRQISPWRADIYENLETLKYELMGLKYSDL
SFEKGTGKYHISQEKYDAIREKEGIGKKSEFKFTLYRNDLILIKDTLNNCERMLRFGSKNDT
SKHYVELKPLEKGTFDSEEEILPVLGKVAKSGQFIKGLNKPNISIYKVRTDVLGNKFFIKKE
GDKPKLDFKNNNKTGPKKKRKVASIFKPEELRQALMPTLEALYRQDPESLPFRQPVDPQLLG
IPDYFDIVKSPMDLSTIKRKLDTGQYQEPWQYVDDIWLMENNAWLYNRKTSRVYKYCSKLSE
VFEQEIDPVMQSLGYCCGRKLEFSPQTLCCYGKQLCTIPRDATYYSYQNRYHFCEKCFNEIQ
GESVSLGDDPSQPQTTINKEQFSKRKNDTLDPELFVECTECGRKMHQICVLHHEIIWPAGFV
CDGCLKKSARTRKENKFSAKRLPSTRLGTFLENRVNDFLRRQNHPESGEVTVRVVHASDKTV
EVKPGMKARFVDSGEMAESFPYRTKALFAFEEIDGVDLCFFGMHVQEYGSDCPPPNQRRVYI
SYLDSVHFFRPKCLRTAVYHEILIGYLEYVKKLGYTTGHIWACPPSEGDDYIFHCHPPDQKI
PKPKRLQEWYKKMLDKAVSERIVHDYKDIFKQATEDRLTSAKELPYFEGDFWPNVLEESIKE
LEQEEEERKREENTSNESTDVTKGDSKNAKKKNNKKTSKNKSSLSRGNKKKPGMPNVSNDLS
QKLYATMEKHKEVFFVIRLIAGPAANSLPPIVDPDPLIPCDLMDGRDAFLTLARDKHLEFSS
LRRAQWSTMCMLVELHTQSQD
Streptococcus lutetiensis dCas9-p300, DNA
SEQ ID NO: 268
ATGTCAAATGGCAAAATCTTAGGCTTGGCCATCGGGGTGGCCAGCGTCGGGGTTGGCATAAT
TGATGCCAAAACCGGCAACGTGATCCACGCAAATAGCAGGCTGTTTAGCGCCGCCAACGCCG
AGAACAATGCTGAGCGGAGGGGATTCCGCGGCGCACGTAGGCTCACGAGGCGCAAAAAACAT
AGAGTGAAGCGGGTCCGTGACCTGTTTGAAAAGTATGATATCTCAACAGATTTCCGCAACTT
AAATCTGAACCCCTACGAGCTCAGGGTGAAAGGCCTGACAGAACAGCTTACCAATGAAGAAC
TCTTCGCAGCTTTAAGAACTATTGCCAAACGGCGCGGCATCTCCTACTTGGATGACGCGGAA
GACGATTCTACCGGAAGCAGCGACTACGCGAAGTCAATCGACGAAAATAGACGTCTTCTGAA
AACCAAAACTCCAGGGCAAATCCAGCTGGAGAGACTGGAGAAGTACGGACAGCTGAGGGGCA
ATTTTACCGTGTATGACGAAAACGGAGAAGCTCACAGACTGATCAATGTTTTTTCCACTTCC
GATTATAAAAACGAAGCCCGGAAGATCCTGGAGACGCAGAGCAACTACAACAAGCAAATCAC
CGATGAGTTCATCGAAGATTACATTGAGATATTAACTCAAAAGCGTAAATACTACCATGGCC
CAGGCAACGAGAAGAGCAGGACCGATTACGGCAGGTTCCGAACAGATGGAACTACCCTGGAG
AACATTTTTGGCATTCTTATTGGAAAATGCTCATTCTATCCAGAGGAATATCGTGCTAGTAA
GGCAAGCTACACCGCCCAAGAATTCAACTTTCTGAATGACCTGAATAATCTGAAGGTCCCCA
CCGAAACGGGCAAGTTATCAACTGAGCAGAAGGAGTATTTAGTGGATTTTGCCAAGAAGTCT
AAGGCTCTGGGAGCGTCTAAGCTTCTGAAGGAGATTGCCAAGATAGTTGATTGCAGCGTTGA
CGACATCAAGGGGTACAGGGTGGATAATAAAGACAAGCCAGATCTGCACACCTTTGAGCCAT
ATAGAAAGTTGAAGTTTAACTTGAGTAGTATCGACATCGATGAACTGTCTAGAGAGACACTC
GACAAACTCGCTGACATTCTTACTCTGAACACAGAACGGGAAGGCATCGAGGATACAATCAA
AAGAAACCTTCCCTCACAGTTTACCGAGGAACAGATAAGCGAGATTGTCCAAATTCGGAAGA
ATCAATCCAGCGCCTTTAACAAGGGTTGGCACTCCTTCTCAGCAAAGTTGATGAACGAGTTA
ATCCCAGAGCTGTACGTGACTTCAGAGGAGCAGATGACAATTCTGACCAGGTTGGAAAAATT
TAAGGTGAACAAGAAGAGCTCCAAAAACACAAAGACCATCGATGAAAAGGAGATTACTGACG
AGATCTATAACCCAGTCGTCGCGAAATCCGTGAGGCAAACTATCAAGATTATCAACGCCGCG
GTGAAAAAGTATGGAGACTTTGACAAAATCGTGATTGAGATGCCACGTGACAAGAATGCAGA
GGATGAGAAAAAATTTATTGACAAAAAGGAGAAGGAAAATAAGAAGGAAAAAGATGATAGCC
TGAAGCGCGCAGCTTTCCTGTATAACGGCACAGACAATTTGCCAGACGGAGTATTTCACGGA
AACAAGGAGCTCAAGACTAAAATTCGCTTATGGTATCAACAAGGCGAGAGGTGCTTGTATAG
CGGCAAACTGATATCCATACACGACCTCGTACACAACAGTAACAAGTTTGAGATTGACGCCA
TCCTTCCACTTAGCCTGAGTTTCGACGACAGCCTGGCAAATAAGGTCTTGGTATATGCTTGG
ACCAATCAGGAGAAGGGGCAAAAAACCCCGTACCAGGTGATAGATAGCATGGACGCGGCATG
GAGTTTTCGGGAAATGAAGGACTACGTTCTCAAACAGAAGAGACTCGGCAAAAAAAAGCGTG
AATACCTGCTGACTACCGAGAACATTGACAAAATCGAAGTCAAAAAAAAGTTCATCGAGCGC
AACCTTGTGGATACCCGCTATGCCTCACGCGTCGTCCTGAACTCTCTGCAGACAGCTCTGAA
AGAACTGGGCAAGGACACCAAAGTGTCTGTCGTTAGGGGTCAATTTACCTCCCAGTTGCGAC
GCAAGTGGAATATCGATAAGTCCAGAGAAACATACCATCATCACGCAGTAGACGCCCTTATC
ATTGCCGCATCTTCTCAGCTTAAACTGTGGCAAAAGCAGGAAAATCCTATGTTTGAGTCTTA
TGGCGAAAATCAGGTCGTCAATAAGGAGACAGGAGAGATCTTATCAATATCCGATGACAAGT
ATAAAGAACTGGTGTTTCAACCACCATACCAAGGGTTTGTCAACACTATCAGCAGTAAAGGC
TTCGAGGATGAGATCTTGTTTTCATATCAGGTGGACAGCAAATTCAACCGGAAAGTTTCTGA
TGCCACCATTTATAGTACTCGCAAAGCGAAACTTGGAAAGGACAAGAAGGATGAGACCTACG
TATTGGGGAAAATCAAGGACATTTACTCTCAGGACGGCTTTGACACCTTCATTAAGCGTTAC
AAAAAGGACAAGACGCAGTTCCTGATGTACCAAAAAGATCCACTGACTTGGGAAAATGTTAT
TGAGGTGATCCTCCGGGATTATCCAAGTGAAAAATTGTCAGAGGACGGCAAAAAAACAGTGA
AGTGCAATCCGTTTGAAGAATATAGGCGAGAGAATGGTCTGATCTGTAAATACTCTAAAAAG
GGCAACGGAACCCCCATCAAGTCCCTGAAATATTACGACAAGAAACTTGGTAACTGCATTGA
CATCACCCCTGAGAAAAGCAAGAACCGCGTGGTGCTGAGGCAGATATCACCTTGGCGCGCTG
ATATCTACTTCAACCTGGAGACCTTGAAATATGAGCTCATGGGCTTGAAATACAGTGACCTG
TCTTTTGAAAAAGGGACCGGGAAGTATCACATTAGCCAGGAAAAGTACGATGCGATTAGAGA
AAAAGAAGGCATTGGCAAAAAGAGCGAGTTTAAGTTTACTTTGTATCGAAACGATCTCATCC
TGATAAAAGATACCCTGAACAATTGTGAGAGGATGCTTAGGTTCGGATCCAAGAACGATACA
TCTAAGCACTACGTGGAACTCAAACCTTTAGAGAAGGGCACCTTTGATTCCGAGGAGGAGAT
CCTTCCAGTGCTGGGCAAGGTTGCGAAATCCGGGCAGTTTATTAAGGGTCTTAACAAACCCA
ATATCTCAATCTATAAGGTGAGGACCGATGTGCTTGGCAACAAATTCTTTATCAAGAAGGAA
GGCGACAAACCCAAGCTGGATTTCAAGAATAATAACAAGACCGGTCCTAAGAAAAAGCGGAA
AGTGGctagCattttcaaaccagaagaactacgacaggcactgatgccaactttggaggcac
tttaccgtcaggatccagaatcccttccctttcgtcaacctgtggaccctcagcttttagga
atccctgattactttgatattgtgaagagccccatggatctttctaccattaagaggaagtt
agacactggacagtatcaggagccctggcagtatgtcgatgatatttggcttatgttcaata
atgcctggttatataaccggaaaacatcacgggtatacaaatactgctccaagctctctgag
gtctttgaacaagaaattgacccagtgatgcaaagccttggatactgttgtggcagaaagtt
ggagttctctccacagacactgtgttgctacggcaaacagttgtgcacaatacctcgtgatg
ccacttattacagttaccagaacaggtatcatttctgtgagaagtgtttcaatgagatccaa
ggggagagcgtttctttgggggatgacccttcccagcctcaaactacaataaataaagaaca
attttccaagagaaaaaatgacacactggatcctgaactgtttgttgaatgtacagagtgcg
gaagaaagatgcatcagatctgtgtccttcaccatgagatcatctggcctgctggattcgtc
tgtgatggctgtttaaagaaaagtgcacgaactaggaaagaaaataagttttctgctaaaag
gttgccatctaccagacttggcacctttctagagaatcgtgtgaatgactttctgaggcgac
agaatcaccctgagtcaggagaggtcactgttagagtagttcatgcttctgacaaaaccgtg
gaagtaaaaccaggcatgaaagcaaggtttgtggacagtggagagatggcagaatcctttcc
ataccgaaccaaagccctctttgcctttgaagaaattgatggtgttgacctgtgcttctttg
gcatgcatgttcaagagtatggctctgactgccctccacccaaccagaggagagtatacata
tcttacctcgatagtgttcatttcttccgtcctaaatgcttgaggactgcagtctatcatga
aatcctaattggatatttagaatatgtcaagaaattaggttacacaacagggcatatttggg
catgtccaccaagtgagggagatgattatatcttccattgccatcctcctgaccagaagata
cccaagcccaagcgactgcaggaatggtacaaaaaaatgcttgacaaggctgtatcagagcg
tattgtccatgactacaaggatatttttaaacaagctactgaagatagattaacaagtgcaa
aggaattgccttatttcgagggtgatttctggcccaatgttctggaagaaagcattaaggaa
ctggaacaggaggaagaagagagaaaacgagaggaaaacaccagcaatgaaagcacagatgt
gaccaagggagacagcaaaaatgctaaaaagaagaataataagaaaaccagcaaaaataaga
gcagcctgagtaggggcaacaagaagaaacccgggatgcccaatgtatctaacgacctctca
cagaaactatatgccaccatggagaagcataaagaggtcttctttgtgatccgcctcattgc
tggccctgctgccaactccctgcctcccattgttgatcctgatcctctcatcccctgcgatc
tgatggatggtcgggatgcgtttctcacgctggcaagggacaagcacctggagttctcttca
ctccgaagagcccagtggtccaccatgtgcatgctggtggagctgcacACGCAGAGCCAGGA
C
Streptococcus parauberis dCas9-p300 amino acid sequence
SEQ ID NO: 269
MQKSYSLGLAIGTNSVGWAVITDDYKVPAKKMKVLGNTDRQTVKKNMIGTLLEDSGETAEAR
RLKRTARRRYTRRINRIKYLQSIFDDEMSKIDSAFFQRIKDSFLVPDDKNDDRHPIFGNIKD
EVDYHKNYPTIYHLRKKLADSDEKADLRLIYLALAHIIKERGHFLIEGDLDSQNTDVNALFL
KLVDTYNLMFEDDKIDTQTIDATVILTEKMSKSRRLENLIAKIPNQKKNTLFGNLISLSLGL
TPNFKANFELSEDAKLQISKDSFEEDLDNLLAQIGDQYADLFIAAKNLSDAILLSDILTVKG
VNTKAPLSASMVQRFNEHQDDLKLLKKLVKVQLPEKYKEIFDIKDKNGYAGYINGKTSQEDF
YKYIKPILSKLKGAESLISKLEREDELRKQRTEDNGSIPHQIHLNELKSIIRRQEKYYPFLK
DKQVRIEKIFTFRIPYFVGPLANGNSSFAWVKRRSNESITPWNFEEVVEQEASAKVFIERMT
NFDTYLPEEKVLPKHSLLYEMFTVYNELTKVKYQAEGMRKPEFLSSEEKIEIVSNLFKKERK
VTVKQLKENYENKIRCLDSITISGVEDKFNASLGTYHDLLNIIKNQKILDDEQNQDSLEDIV
LTLTLFEDEKMIAKRLSKYESIFEPSILKKLKKRHYTGWGRLSQKLINGIRDKQTGKTILDE
LIDDGQANRNFMQLINDPSLDFASIIKGAQEKTIKSEKLEETIANLAGSPAIKKGILQSVKI
VDEVVKVMGYEPSNIVIEMARENQSTHRGINNSRERLRKLEEVHKNIGSKILKEHEISNAQL
QSDRVYLYLLQDGKDMYTGKDLDFDRLSQYDIDAIIPQSFIKDNSIDNIVLTSQESNRGKSD
NVPYIAIVNKMKSYWQHQLKSGAISQRKEDNLTKVERGGLSEYDKAGFIKRQLVETRQITKH
VAQILNNRFNNNVDNSSKNKRPVKIITLKSKMVSDFRKEFGFYKIREVNDYHHAHDAYLNAV
VGTALLKKYPKLEAEFVYGDYKHYDLASLVVKSDTSLGKATAKMFFYSNIMNFFKKEVRLAD
GTVITRPQIETNTETGEIVWDKVKDIKTIRKVLSIPQINVVKKTEVQTGGFSKESILPKGDS
DKLIPRKNNWDPKKYGGFDSPIIAYSVLVVAKVAKGKSQKTKSVKELVGITIMEQNEFEKDR
ITFLEKKGYQDIQESLIIKLPKFSLFELENGRKRLLASAKELQKGNELSLPNKYIQFLYLAS
RYTSFSGKEEDREKHRHFVESHLHYFDEIKDIIADESRRYILADANLEKILTLYNEKNQFSI
EEQATNMLNLFTFTGLGAPATLKFFNVDIDRKRYTSSTEILNSTLIRQSITGLYETRIDLSK
IGGDTGPKKKRKVASIFKPEELRQALMPTLEALYRQDPESLPFRQPVDPQLLGIPDYFDIVK
SPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDD
PSQPQTTINKEQFSKRKNDTLDPELFVECTECGRKMHQICVLHHEIIWPAGFVCDGCLKKSA
RTRKENKFSAKRLPSTRLGTFLENRVNDFLRRQNHPESGEVTVRVVHASDKTVEVKPGMKAR
FVDSGEMAESFPYRTKALFAFEEIDGVDLCFFGMHVQEYGSDCPPPNQRRVYISYLDSVHFF
RPKCLRTAVYHEILIGYLEYVKKLGYTTGHIWACPPSEGDDYIFHCHPPDQKIPKPKRLQEW
YKKMLDKAVSERIVHDYKDIFKQATEDRLTSAKELPYFEGDFWPNVLEESIKELEQEEEERK
REENTSNESTDVTKGDSKNAKKKNNKKTSKNKSSLSRGNKKKPGMPNVSNDLSQKLYATMEK
HKEVFFVIRLIAGPAANSLPPIVDPDPLIPCDLMDGRDAFLTLARDKHLEFSSLRRAQWSTM
CMLVELHTQSQD
Streptococcus parauberis dCas9-p300, DNA
SEQ ID NO: 270
ATGCAAAAGAGCTACTCTCTCGGGTTAGCAATCGGAACAAATAGTGTGGGATGGGCGGTGAT
TACGGACGATTATAAGGTGCCAGCCAAAAAGATGAAGGTTCTTGGCAATACGGACCGGCAGA
CGGTGAAGAAGAACATGATTGGCACTCTGCTGTTTGATAGTGGAGAAACCGCTGAGGCCCGG
AGACTCAAAAGGACTGCTAGGCGACGGTATACGCGGCGTATTAACCGCATTAAATATCTTCA
GTCTATATTTGATGATGAGATGTCAAAGATCGACAGCGCGTTTTTTCAGCGAATTAAAGATT
CCTTCCTTGTCCCAGATGACAAGAATGACGATAGACATCCGATTTTTGGTAACATTAAGGAC
GAGGTTGACTACCATAAGAACTATCCGACAATTTATCACCTGCGCAAGAAGCTGGCAGACTC
CGACGAGAAGGCAGACCTTAGACTGATTTACCTCGCTCTGGCTCACATCATAAAATTTCGAG
GACACTTCTTGATAGAAGGAGATCTCGACAGCCAGAATACTGATGTTAACGCCCTGTTCCTG
AAATTAGTCGACACCTACAACCTCATGTTTGAGGATGACAAAATCGATACGCAGACTATTGA
CGCAACAGTGATTTTAACTGAGAAGATGAGTAAGTCACGGCGACTTGAGAACTTGATAGCCA
AGATACCTAATCAAAAGAAGAATACCCTCTTCGGAAATCTGATTTCACTCAGTCTTGGCCTG
ACACCTAACTTTAAAGCTAATTTTGAATTGAGCGAGGACGCGAAGCTTCAAATCTCTAAGGA
CTCCTTCGAAGAAGATTTGGATAACCTCCTCGCCCAGATCGGTGACCAATACGCTGACCTGT
TTATAGCAGCGAAGAATTTGTCTGACGCTATCCTCCTGTCTGATATCCTTACTGTGAAGGGC
GTGAATACAAAGGCACCCTTATCCGCCAGTATGGTCCAGCGGTTCAACGAACATCAAGACGA
CCTGAAGTTGCTCAAAAAACTCGTGAAGGTGCAACTGCCCGAGAAATACAAAGAAATTTTCG
ACATTAAAGACAAAAATGGGTACGCTGGGTATATTAACGGTAAGACATCCCAGGAGGACTTT
TACAAATATATCAAGCCTATCTTAAGCAAGCTGAAAGGGGCGGAGTCCCTTATCTCTAAATT
GGAGAGAGAAGACTTTTTGCGGAAGCAGAGAACCTTCGATAATGGATCCATTCCCCACCAGA
TTCACTTGAATGAGCTCAAATCCATCATCCGACGACAGGAGAAGTATTATCCCTTTCTGAAG
GATAAACAGGTGCGGATTGAAAAGATCTTCACCTTTAGAATACCATATTTTGTTGGACCATT
GGCTAACGGGAACTCTTCATTTGCTTGGGTTAAGCGAAGATCTAACGAATCTATTACACCAT
GGAACTTTGAGGAAGTCGTTGAGCAGGAGGCCAGCGCCAAGGTCTTCATAGAGCGGATGACT
AATTTTGATACCTACCTGCCAGAGGAGAAGGTCCTTCCCAAGCACTCTTTGCTCTATGAAAT
GTTCACTGTATACAACGAACTGACTAAAGTAAAGTATCAGGCCGAGGGCATGAGAAAGCCCG
AATTCTTGAGTTCAGAAGAAAAGATTGAGATTGTGTCCAACCTGTTTAAGAAGGAGAGAAAG
GTGACAGTCAAGCAGCTTAAGGAAAATTATTTCAATAAGATAAGATGTCTTGACTCAATCAC
CATCAGTGGGGTTGAAGACAAGTTCAACGCATCACTGGGTACTTACCACGATTTACTTAACA
TTATTAAGAACCAGAAGATTCTGGACGATGAGCAGAACCAGGACTCCCTCGAGGATATTGTG
TTGACTCTGACACTGTTCGAGGACGAAAAAATGATCGCGAAGAGGCTGTCAAAGTATGAATC
CATTTTCGAGCCCAGCATTTTGAAGAAATTAAAAAAGCGCCACTATACTGGTTGGGGCCGTT
TATCCCAGAAGCTCATCAACGGCATCCGTGATAAACAGACCGGAAAGACCATCCTGGACTTC
CTGATCGACGATGGCCAGGCGAATCGAAATTTCATGCAATTGATTAACGATCCCTCTCTGGA
CTTTGCGTCAATAATCAAGGGGGCCCAGGAAAAGACGATAAAGAGCGAGAAGCTCGAAGAGA
CCATCGCTAATCTCGCCGGATCTCCCGCTATCAAGAAAGGCATCTTACAGTCTGTGAAGATT
GTAGATGAAGTGGTGAAAGTGATGGGCTATGAACCTAGCAACATTGTCATAGAAATGGCCAG
GGAAAATCAGTCAACCCACCGAGGCATAAATAACTCTAGGGAACGATTACGAAAGCTGGAGG
AGGTCCACAAGAACATTGGCTCCAAGATCTTGAAAGAGCACGAAATTAGCAATGCCCAACTC
CAGAGTGACCGAGTGTACTTGTATCTGTTGCAGGATGGAAAAGATATGTACACCGGTAAGGA
CCTCGATTTCGATCGGCTCTCTCAGTACGATATTGATGCAATCATACCACAGTCCTTTATTA
AGGACAACAGTATTGATAATATCGTCCTGACATCTCAGGAAAGCAATAGAGGAAAGTCAGAT
AATGTGCCCTACATTGCAATCGTGAATAAGATGAAATCATACTGGCAACACCAGCTGAAATC
TGGGGCTATCAGCCAGCGGAAATTTGATAATTTAACTAAGGTGGAGCGGGGCGGCCTCAGCG
AGTATGATAAGGCAGGTTTTATCAAACGTCAGCTCGTTGAGACACGTCAGATAACAAAGCAC
GTGGCACAAATCCTTAATAATAGATTCAACAACAACGTCGATAACAGTAGCAAGAACAAAAG
ACCTGTCAAGATAATCACATTAAAATCTAAAATGGTGTCTGATTTCCGTAAGGAATTCGGCT
TCTATAAAATTAGGGAGGTAAATGACTATCATCACGCCCACGACGCCTACCTCAACGCCGTT
GTCGGGACAGCCCTGTTGAAAAAATATCCAAAGCTGGAGGCAGAATTCGTGTACGGCGATTA
CAAGCACTATGACTTGGCCTCACTGGTTGTCAAGAGCGACACTAGTCTGGGCAAAGCCACTG
CAAAAATGTTTTTTTATTCTAATATCATGAACTTCTTCAAAAAGGAGGTCAGACTGGCAGAT
GGCACCGTGATCACAAGACCTCAGATAGAGACTAATACGGAAACTGGCGAGATCGTGTGGGA
TAAGGTAAAGGACATTAAAACAATTAGGAAGGTGCTGTCTATACCCCAGATCAACGTGGTTA
AAAAGACTGAAGTCCAAACTGGGGGTTTCTCAAAGGAAAGCATCCTGCCCAAGGGCGATAGC
GATAAGCTTATTCCTAGAAAGAACAATTGGGATCCAAAGAAGTATGGTGGCTTTGATTCTCC
GATCATTGCCTATTCTGTCTTAGTGGTCGCAAAAGTGGCGAAGGGCAAAAGCCAGAAGACAA
AGAGTGTCAAAGAACTTGTCGGAATTACTATCATGGAACAGAACGAGTTCGAAAAGGATCGG
ATTACATTCCTTGAGAAAAAAGGATACCAGGATATTCAGGAATCACTGATCATTAAGCTGCC
CAAGTTCAGCTTGTTTGAGCTTGAAAACGGGAGAAAGCGTCTGCTCGCCAGCGCAAAAGAGC
TCCAGAAGGGAAATGAGCTGTCATTGCCAAACAAGTACATCCAATTTTTGTATCTCGCCTCC
AGATATACTAGCTTTAGCGGCAAGGAGGAAGATAGAGAGAAGCACAGACACTTCGTGGAATC
TCACCTGCACTACTTTGATGAGATTAAAGACATAATTGCCGATTTTTCTCGACGCTATATTC
TGGCAGATGCGAACCTTGAAAAAATTCTCACGCTGTACAATGAGAAAAATCAGTTCTCAATT
GAAGAGCAGGCTACCAACATGCTGAACCTCTTCACCTTCACGGGACTGGGAGCCCCTGCCAC
CCTGAAATTTTTCAACGTGGACATTGATCGGAAGCGATACACTTCCTCCACCGAGATTCTGA
ATAGTACCCTCATTAGACAGAGTATTACCGGACTCTACGAGACAAGGATTGACCTCTCCAAA
ATTGGCGGGGACACCGGTCCTAAGAAAAAGCGGAAAGTGGctagCattttcaaaccagaaga
actacgacaggcactgatgccaactttggaggcactttaccgtcaggatccagaatcccttc
cctttcgtcaacctgtggaccctcagcttttaggaatccctgattactttgatattgtgaag
agccccatggatctttctaccattaagaggaagttagacactggacagtatcaggagccctg
gcagtatgtcgatgatatttggcttatgttcaataatgcctggttatataaccggaaaacat
cacgggtatacaaatactgctccaagctctctgaggtctttgaacaagaaattgacccagtg
atgcaaagccttggatactgttgtggcagaaagttggagttctctccacagacactgtgttg
ctacggcaaacagttgtgcacaatacctcgtgatgccacttattacagttaccagaacaggt
atcatttctgtgagaagtgtttcaatgagatccaaggggagagcgtttctttgggggatgac
ccttcccagcctcaaactacaataaataaagaacaattttccaagagaaaaaatgacacact
ggatcctgaactgtttgttgaatgtacagagtgcggaagaaagatgcatcagatctgtgtcc
ttcaccatgagatcatctggcctgctggattcgtctgtgatggctgtttaaagaaaagtgca
cgaactaggaaagaaaataagttttctgctaaaaggttgccatctaccagacttggcacctt
tctagagaatcgtgtgaatgactttctgaggcgacagaatcaccctgagtcaggagaggtca
ctgttagagtagttcatgcttctgacaaaaccgtggaagtaaaaccaggcatgaaagcaagg
tttgtggacagtggagagatggcagaatcctttccataccgaaccaaagccctctttgcctt
tgaagaaattgatggtgttgacctgtgcttctttggcatgcatgttcaagagtatggctctg
actgccctccacccaaccagaggagagtatacatatcttacctcgatagtgttcatttcttc
cgtcctaaatgcttgaggactgcagtctatcatgaaatcctaattggatatttagaatatgt
caagaaattaggttacacaacagggcatatttgggcatgtccaccaagtgagggagatgatt
atatcttccattgccatcctcctgaccagaagatacccaagcccaagcgactgcaggaatgg
tacaaaaaaatgcttgacaaggctgtatcagagcgtattgtccatgactacaaggatatttt
taaacaagctactgaagatagattaacaagtgcaaaggaattgccttatttcgagggtgatt
tctggcccaatgttctggaagaaagcattaaggaactggaacaggaggaagaagagagaaaa
cgagaggaaaacaccagcaatgaaagcacagatgtgaccaagggagacagcaaaaatgctaa
aaagaagaataataagaaaaccagcaaaaataagagcagcctgagtaggggcaacaagaaga
aacccgggatgcccaatgtatctaacgacctctcacagaaactatatgccaccatggagaag
cataaagaggtcttctttgtgatccgcctcattgctggccctgctgccaactccctgcctcc
cattgttgatcctgatcctctcatcccctgcgatctgatggatggtcgggatgcgtttctca
cgctggcaagggacaagcacctggagttctcttcactccgaagagcccagtggtccaccatg
tgcatgctggtggagctgcacACGCAGAGCCAGGAC
Streptococcus dysgalactiae dCas9-p300 amino acid
SEQ ID NO: 271
MDKKYSIGLAIGTNSVGWAVITDDYKVPSKKFKVLGNTDRHSIKKNLIGALLFDSGETAEAT
RLKRTARRRYTRRKNRIRYLQEIFSSEMSKVDDSFFHRLEESFLVEEDKKHERHPIFGNIVD
EVAYHEKYPTIYHLRKKLADSTDKADLRLIYLALAHMIKFRGHFLIEGDLNPDNSDMDKLFI
QLVQTYNQLFEENPINASRVDAKAILSARLSKSRRLENLIAQLPGEKRNGLEGNLIALSLGL
TPNFKSNFDLAEDAKLQLSKDTYDDDLDNLLAQIGDQYADLFLAAKNLSDAILLSDILRVNS
EITKAPLSASMIKRYDEHHQDLTLLKALVRQQLPEKYKEIFFDQSKNGYAGYIDGGASQEEF
YKFIKPILEKMDGTEELLAKLNREDLLRKQRTFDNGSIPHQIHLGELHAILRRQEDFYPELK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPEFLSGKQKEAIVDLLFKTNRK
VTVKQLKEDYFKKIECFDSVEISGVEDRENASLGTYHDLLKIIKDKDELDNEENEDILEDIV
LTLTLFEDKEMIEERLKKYANLFDDKVMKQLKRRHYTGWGRLSRKLINGIRDKQSGKTILDE
LKSDGFANRNFMQLINDDSLTFKEAIQKAQVSGQGHSLHEQIANLAGSPAIKKGILQSVKVV
DELVKVMGHKPENIVIEMARENQTTQKGQKNSRERMKRIEEGIKELGSQILKEHPVENTQLQ
NEKLYLYYLQNGRDMYVDQELDINRLSDYDVDAIVPQSFIKDDSIDNKILTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYTKLESEFVYGDYKVYDVRKMIAKSEQEIGKATAKRFFYSNIMNFFKTEITLANG
EIRKRPLIETNEETGEIVWDKGRDFATVRKVLSMPQVNIVKKTEVQTGALTNESIYARGSFD
KLISRKHRFESSKYGGFGSPTVTYSVLVVAKSKVQDGKVKKIKTGKELIGMTLLDKLVFEKN
PLKFIEDKGYGNVQIDKCIKLPKYSLFEFENGTRRMLASVMANNNSRGDLQKANEMFLPAKL
VILLYHAHKIESSKELEHEAYILDHYNDLYQLLSYIERFASLYVDVEKNISKVKELESNIES
YSISEICSSVINLLTLTASGAPADEKELGTTIPRKRYGSPQSILSSTLIHQSITGLYETRID
LSQLGGDTGPKKKRKVASIFKPEELRQALMPTLEALYRQDPESLPFRQPVDPQLLGIPDYFD
IVKSPMDLSTIKRKLDTGQYQEPWQYVDDIWLMENNAWLYNRKTSRVYKYCSKLSEVFEQEI
DPVMQSLGYCCGRKLEFSPQTLCCYGKQLCTIPRDATYYSYQNRYHFCEKCFNEIQGESVSL
GDDPSQPQTTINKEQFSKRKNDTLDPELFVECTECGRKMHQICVLHHEIIWPAGFVCDGCLK
KSARTRKENKFSAKRLPSTRLGTFLENRVNDFLRRQNHPESGEVTVRVVHASDKTVEVKPGM
KARFVDSGEMAESFPYRTKALFAFEEIDGVDLCFFGMHVQEYGSDCPPPNQRRVYISYLDSV
HFFRPKCLRTAVYHEILIGYLEYVKKLGYTTGHIWACPPSEGDDYIFHCHPPDQKIPKPKRL
QEWYKKMLDKAVSERIVHDYKDIFKQATEDRLTSAKELPYFEGDFWPNVLEESIKELEQEEE
ERKREENTSNESTDVTKGDSKNAKKKNNKKTSKNKSSLSRGNKKKPGMPNVSNDLSQKLYAT
MEKHKEVFFVIRLIAGPAANSLPPIVDPDPLIPCDLMDGRDAFLTLARDKHLEFSSLRRAQW
STMCMLVELHTQSQD
Streptococcus dysgalactiae dCas9-p300, DNA
SEQ ID NO: 272
ATGGATAAGAAGTACTCCATTGGACTGGCAATTGGGACAAATTCAGTGGGATGGGCTGTGAT
AACGGATGATTATAAAGTGCCATCTAAGAAGTTTAAAGTGCTGGGGAACACAGACAGACACT
CAATCAAAAAGAATTTGATTGGGGCCCTCCTCTTCGACTCAGGTGAGACCGCTGAAGCTACT
CGCCTCAAGAGAACAGCGAGACGGCGGTATACTCGTAGAAAGAACCGCATTCGCTACCTGCA
AGAGATATTTAGCAGCGAAATGAGTAAGGTGGACGATAGCTTCTTCCACAGACTGGAGGAGA
GCTTTCTTGTGGAGGAGGACAAGAAACACGAGCGCCATCCCATCTTTGGTAATATTGTGGAC
GAGGTGGCCTATCATGAGAAGTATCCAACAATTTACCACCTTAGAAAGAAGTTGGCAGATTC
CACCGACAAAGCTGACCTCCGGCTGATCTACCTTGCTCTCGCACATATGATTAAATTCCGGG
GACACTTCTTGATTGAAGGCGACCTTAACCCCGACAACTCAGATATGGACAAGCTCTTCATC
CAGCTCGTACAAACCTACAATCAGCTTTTCGAGGAAAACCCAATTAACGCTTCCAGGGTCGA
CGCAAAAGCGATACTGTCTGCTCGTCTTAGTAAGTCCCGGCGGCTCGAGAACTTAATTGCAC
AGTTGCCCGGCGAAAAGCGTAATGGACTGTTTGGGAATCTCATTGCCCTTTCCCTTGGACTG
ACTCCAAATTTCAAGTCAAATTTCGATCTCGCTGAGGACGCAAAACTGCAGCTGTCTAAGGA
CACTTACGACGATGACCTGGACAACCTGCTGGCTCAGATTGGCGACCAGTACGCCGATTTAT
TCCTCGCCGCAAAAAACCTTTCTGATGCCATCCTGCTGAGCGATATTCTTAGAGTTAACAGT
GAGATTACAAAAGCCCCCCTGAGTGCCTCCATGATTAAGCGCTATGACGAACACCACCAAGA
CTTGACTCTCCTGAAAGCTTTAGTACGGCAACAGCTCCCCGAGAAATATAAGGAGATCTTTT
TCGATCAATCCAAGAACGGATACGCGGGATATATAGATGGAGGGGCTAGCCAAGAGGAATTT
TACAAGTTCATCAAACCAATTTTAGAAAAGATGGACGGAACAGAAGAATTATTGGCCAAGCT
GAATCGGGAGGATCTGCTGAGAAAGCAGAGAACATTCGATAACGGCTCCATACCCCACCAGA
TCCACCTCGGAGAATTACACGCAATTCTTAGACGCCAGGAGGATTTCTACCCCTTCCTGAAA
GACAATCGAGAGAAGATTGAAAAAATACTGACATTTCGGATCCCCTATTACGTGGGTCCTCT
GGCCCGAGGGAATAGTCGGTTCGCCTGGATGACACGTAAGTCAGAAGAGACGATTACCCCCT
GGAATTTTGAGGAAGTGGTTGATAAAGGCGCCAGCGCTCAGTCTTTCATCGAGCGTATGACT
AATTTTGACAAAAACTTGCCCAACGAGAAAGTCCTCCCCAAACACTCCTTACTTTATGAGTA
CTTCACCGTCTATAACGAGCTTACAAAAGTTAAGTACGTAACTGAGGGTATGAGGAAACCAG
AGTTCCTCAGCGGGAAACAAAAGGAGGCCATTGTGGATCTGCTTTTCAAAACAAACAGGAAG
GTTACCGTGAAACAATTAAAGGAGGATTACTTTAAGAAAATCGAGTGCTTCGATAGCGTCGA
GATATCTGGAGTAGAAGACAGGTTCAACGCGTCCCTGGGTACCTACCACGATCTGCTGAAAA
TAATCAAGGACAAGGACTTCCTCGATAATGAGGAAAATGAAGATATCCTGGAGGACATCGTG
CTTACTCTGACACTGTTTGAAGACAAAGAGATGATAGAGGAGAGGCTGAAGAAATATGCAAA
TCTTTTCGATGATAAAGTTATGAAACAGCTTAAGCGAAGGCATTACACCGGGTGGGGGAGGC
TGAGCCGGAAGCTTATCAATGGGATCAGGGACAAGCAGAGCGGGAAGACTATATTGGATTTT
CTGAAGTCTGATGGGTTTGCAAATAGGAACTTCATGCAGCTCATTAATGACGATTCACTGAC
ATTTAAGGAGGCTATTCAGAAGGCTCAAGTAAGTGGACAGGGGCATAGCCTGCACGAACAGA
TTGCTAATCTCGCCGGATCTCCAGCAATTAAGAAGGGCATCCTGCAGAGTGTTAAAGTTGTG
GACGAGCTGGTCAAGGTGATGGGCCACAAGCCTGAAAATATAGTTATTGAGATGGCGAGGGA
AAACCAAACAACTCAGAAAGGACAAAAAAACTCCCGCGAACGAATGAAAAGGATCGAAGAGG
GCATTAAAGAATTGGGCTCCCAGATTCTCAAAGAACATCCTGTTGAAAATACCCAGCTGCAG
AACGAGAAGCTGTATCTGTATTATCTGCAGAACGGGAGAGATATGTACGTCGACCAGGAGCT
GGACATTAACCGATTGTCTGACTACGATGTCGACGCAATCGTTCCGCAAAGCTTCATAAAGG
ATGATTCCATCGACAATAAAATTCTCACTCGGAGCGACAAAAATCGAGGAAAGTCTGACAAT
GTGCCCAGCGAAGAGGTGGTAAAGAAGATGAAGAACTACTGGAGACAGCTTCTGAATGCTAA
ACTGATTACTCAACGTAAGTTCGACAATCTGACAAAGGCTGAAAGGGGGGGTCTGAGCGAGC
TGGATAAGGCTGGGTTCATTAAAAGGCAGTTGGTCGAAACCCGACAAATCACCAAGCATGTT
GCTCAGATCTTGGACTCAAGAATGAACACAAAATATGATGAAAACGATAAACTGATTAGGGA
GGTGAAGGTGATCACTCTTAAGAGCAAGTTAGTCTCAGACTTCAGGAAAGATTTTCAGTTCT
ATAAGGTGCGGGAGATTAACAACTATCATCATGCCCACGACGCGTATCTCAACGCGGTTGTG
GGAACCGCCCTGATCAAAAAGTACACTAAGCTGGAGAGCGAGTTTGTTTATGGAGATTATAA
AGTGTACGACGTAAGGAAGATGATCGCGAAGTCAGAGCAGGAGATCGGTAAAGCTACCGCAA
AGCGCTTCTTCTACAGTAACATTATGAACTTCTTCAAGACAGAGATTACGCTCGCCAATGGC
GAGATACGGAAGAGACCCCTGATTGAGACTAACGAAGAAACAGGCGAGATCGTTTGGGACAA
AGGAAGAGATTTCGCTACAGTGCGGAAAGTGCTCTCTATGCCCCAGGTGAATATCGTCAAGA
AGACAGAAGTGCAGACCGGAGCGTTAACCAACGAGAGCATATATGCACGCGGCTCCTTTGAT
AAGCTGATCTCCAGGAAGCACAGGTTCGAGTCCTCCAAGTACGGGGGCTTCGGCAGCCCAAC
TGTTACTTACTCCGTCCTGGTGGTGGCCAAAAGCAAAGTCCAAGACGGGAAGGTCAAAAAGA
TCAAGACAGGGAAAGAGCTGATTGGCATGACACTGTTGGACAAGTTGGTGTTCGAGAAAAAC
CCCCTGAAATTTATAGAAGACAAGGGGTACGGAAACGTGCAGATCGATAAGTGCATTAAGCT
GCCTAAGTACTCTTTATTCGAGTTCGAAAACGGCACCCGTCGGATGTTAGCCTCCGTCATGG
CGAATAATAACAGCAGGGGCGACTTGCAGAAAGCTAACGAAATGTTTCTGCCTGCCAAGTTG
GTGACATTGCTGTATCACGCCCACAAGATTGAATCAAGCAAAGAGCTGGAGCACGAGGCATA
CATCCTTGATCATTACAATGATTTGTATCAGCTCCTGTCTTACATCGAACGGTTCGCCAGCC
TGTATGTGGACGTAGAGAAGAACATATCTAAGGTAAAGGAGTTGTTTTCCAACATCGAATCC
TACAGCATCAGTGAGATCTGCTCCTCTGTGATTAATCTCTTAACTTTAACAGCTAGCGGGGC
CCCGGCCGACTTTAAATTCTTAGGTACAACGATCCCGCGCAAGAGGTACGGCTCCCCCCAAT
CAATTCTCTCCAGCACACTGATTCACCAGAGCATCACCGGCTTATATGAAACGAGGATTGAC
CTGAGTCAGCTTGGTGGCGACACCGGTCCTAAGAAAAAGCGGAAAGTGGctagCattttcaa
accagaagaactacgacaggcactgatgccaactttggaggcactttaccgtcaggatccag
aatcccttccctttcgtcaacctgtggaccctcagcttttaggaatccctgattactttgat
attgtgaagagccccatggatctttctaccattaagaggaagttagacactggacagtatca
ggagccctggcagtatgtcgatgatatttggcttatgttcaataatgcctggttatataacc
ggaaaacatcacgggtatacaaatactgctccaagctctctgaggtctttgaacaagaaatt
gacccagtgatgcaaagccttggatactgttgtggcagaaagttggagttctctccacagac
actgtgttgctacggcaaacagttgtgcacaatacctcgtgatgccacttattacagttacc
agaacaggtatcatttctgtgagaagtgtttcaatgagatccaaggggagagcgtttctttg
ggggatgacccttcccagcctcaaactacaataaataaagaacaattttccaagagaaaaaa
tgacacactggatcctgaactgtttgttgaatgtacagagtgcggaagaaagatgcatcaga
tctgtgtccttcaccatgagatcatctggcctgctggattcgtctgtgatggctgtttaaag
aaaagtgcacgaactaggaaagaaaataagttttctgctaaaaggttgccatctaccagact
tggcacctttctagagaatcgtgtgaatgactttctgaggcgacagaatcaccctgagtcag
gagaggtcactgttagagtagttcatgcttctgacaaaaccgtggaagtaaaaccaggcatg
aaagcaaggtttgtggacagtggagagatggcagaatcctttccataccgaaccaaagccct
ctttgcctttgaagaaattgatggtgttgacctgtgcttctttggcatgcatgttcaagagt
atggctctgactgccctccacccaaccagaggagagtatacatatcttacctcgatagtgtt
catttcttccgtcctaaatgcttgaggactgcagtctatcatgaaatcctaattggatattt
agaatatgtcaagaaattaggttacacaacagggcatatttgggcatgtccaccaagtgagg
gagatgattatatcttccattgccatcctcctgaccagaagatacccaagcccaagcgactg
caggaatggtacaaaaaaatgcttgacaaggctgtatcagagcgtattgtccatgactacaa
ggatatttttaaacaagctactgaagatagattaacaagtgcaaaggaattgccttatttcg
agggtgatttctggcccaatgttctggaagaaagcattaaggaactggaacaggaggaagaa
gagagaaaacgagaggaaaacaccagcaatgaaagcacagatgtgaccaagggagacagcaa
aaatgctaaaaagaagaataataagaaaaccagcaaaaataagagcagcctgagtaggggca
acaagaagaaacccgggatgcccaatgtatctaacgacctctcacagaaactatatgccacc
atggagaagcataaagaggtcttctttgtgatccgcctcattgctggccctgctgccaactc
cctgcctcccattgttgatcctgatcctctcatcccctgcgatctgatggatggtcgggatg
cgtttctcacgctggcaagggacaagcacctggagttctcttcactccgaagagcccagtgg
tccaccatgtgcatgctggtggagctgcacACGCAGAGCCAGGAC
Streptococcus uberis PAM
SEQ ID NO: 273
NNA(A/G)TAN with slight preference for G, C, or T in final position
Streptococcus uberis PAM
SEQ ID NO: 274
NNAATA
Streptococcus gallolyticus PAM
SEQ ID NO: 275
NNG(T/C)(G/A)AN, with slight preference for A in final position
Streptococcus gallolyticus PAM
SEQ ID NO: 276
NNGTAAA
Streptococcus iniae PAM
SEQ ID NO: 277
NNGGNNN
Streptococcus lutentiensis PAM
SEQ ID NO: 278
NNAAAAN with slight preference for A in final position
Streptococcus lutentiensis PAM
SEQ ID NO: 279
NNAAAAA
Streptococcus dysgalactiae PAM
SEQ ID NO: 280
NNGGNTN with slight preference for C in final position
Streptococcus parasanguinis PAM
SEQ ID NO: 281
NNAA(A/G)GN with slight preference for G, C, or T in final position
Streptococcus parasanguinis PAM
SEQ ID NO: 282
NNAAAG

1. A Clustered Regularly Interspaced Short Palindromic Repeats associated (Cas) protein comprising an amino acid sequence having at least 80%, 85%, 90%, 95%, or 98% or greater identity to at least one of SEQ ID NOs: 57, 241, 243, 245, 247, 249, 251, 235, or 223, or any fragment thereof, or wherein the Cas protein is from Streptococcus uberis, Streptococcus agalactiae, Streptococcus gallolyticus, Streptococcus iniae, Streptococcus lutetiensis, Streptococcus mutans, Streptococcus parauberis, Streptococcus dysgalactiae, or Streptococcus parasanguinis.

2. The Cas protein of claim 1, wherein the Cas protein comprises an amino acid sequence having one, two, three, four, five or more changes selected from amino acid substitutions, insertions, or deletions, relative to SEQ ID NO: 57, or any fragment thereof,
or wherein the Cas protein comprises the amino acid sequence of SEQ ID NO: 57,
or wherein the Cas protein is encoded by a polynucleotide comprising a sequence having at least 80%, 85%, 90%, 95%, or 98% or greater identity to SEQ ID NO: 58, or any fragment thereof,
or wherein the Cas protein is encoded by a polynucleotide comprising a sequence having one, two, three, four, five or more changes selected from nucleotide substitutions, insertions, or deletions, relative to SEQ ID NO: 58, or any fragment thereof,
or wherein the Cas protein is encoded by a polynucleotide comprising the sequence of SEQ ID NO: 58.

3. The Cas protein of claim 1, wherein the Cas protein comprises an amino acid sequence having one, two, three, four, five or more changes selected from amino acid substitutions, insertions, or deletions, relative to SEQ ID NO: 223, or any fragment thereof,
or wherein the Cas protein comprises the amino acid sequence of SEQ ID NO: 223,
or wherein the Cas protein is encoded by a polynucleotide comprising a sequence having at least 80%, 85%, 90%, 95%, or 98% or greater identity to SEQ ID NO: 224, or any fragment thereof,
or wherein the Cas protein is encoded by a polynucleotide comprising a sequence having one, two, three, four, five or more changes selected from nucleotide substitutions, insertions, or deletions, relative to SEQ ID NO: 224, or any fragment thereof,
or wherein the Cas protein is encoded by a polynucleotide comprising the sequence of SEQ ID NO: 224.

4. The Cas protein of claim 1, wherein the Cas protein comprises an amino acid sequence having one, two, three, four, five or more changes selected from amino acid substitutions, insertions, or deletions, relative to SEQ ID NO: 241, or any fragment thereof,
or wherein the Cas protein comprises the amino acid sequence of SEQ ID NO: 241,
or wherein the Cas protein is encoded by a polynucleotide comprising a sequence having at least 80%, 85%, 90%, 95%, or 98% or greater identity to SEQ ID NO: 242, or any fragment thereof,
or wherein the Cas protein is encoded by a polynucleotide comprising a sequence having one, two, three, four, five or more changes selected from nucleotide substitutions, insertions, or deletions, relative to SEQ ID NO: 242, or any fragment thereof,
or wherein the Cas protein is encoded by a polynucleotide comprising the sequence of SEQ ID NO: 242.

5. The Cas protein of claim 1, wherein the Cas protein comprises an amino acid sequence having one, two, three, four, five or more changes selected from amino acid substitutions, insertions, or deletions, relative to SEQ ID NO: 243, or any fragment thereof,
or wherein the Cas protein comprises the amino acid sequence of SEQ ID NO: 243,
or wherein the Cas protein is encoded by a polynucleotide comprising a sequence having at least 80%, 85%, 90%, 95%, or 98% or greater identity to SEQ ID NO: 244, or any fragment thereof,
or wherein the Cas protein is encoded by a polynucleotide comprising a sequence having one, two, three, four, five or more changes selected from nucleotide substitutions, insertions, or deletions, relative to SEQ ID NO: 244, or any fragment thereof,
or wherein the Cas protein is encoded by a polynucleotide comprising the sequence of SEQ ID NO: 244.

6. The Cas protein of claim 1, wherein the Cas protein comprises an amino acid sequence having one, two, three, four, five or more changes selected from amino acid substitutions, insertions, or deletions, relative to SEQ ID NO: 245, or any fragment thereof,
or wherein the Cas protein comprises the amino acid sequence of SEQ ID NO: 245,
or wherein the Cas protein is encoded by a polynucleotide comprising a sequence having at least 80%, 85%, 90%, 95%, or 98% or greater identity to SEQ ID NO: 246, or any fragment thereof,
or wherein the Cas protein is encoded by a polynucleotide comprising a sequence having one, two, three, four, five or more changes selected from nucleotide substitutions, insertions, or deletions, relative to SEQ ID NO: 246, or any fragment thereof,
or wherein the Cas protein is encoded by a polynucleotide comprising the sequence of SEQ ID NO: 246.

7. The Cas protein of claim 1, wherein the Cas protein comprises an amino acid sequence having one, two, three, four, five or more changes selected from amino acid substitutions, insertions, or deletions, relative to SEQ ID NO: 247, or any fragment thereof,
or wherein the Cas protein comprises the amino acid sequence of SEQ ID NO: 247,
or wherein the Cas protein is encoded by a polynucleotide comprising a sequence having at least 80%, 85%, 90%, 95%, or 98% or greater identity to SEQ ID NO: 248, or any fragment thereof,
or wherein the Cas protein is encoded by a polynucleotide comprising a sequence having one, two, three, four, five or more changes selected from nucleotide substitutions, insertions, or deletions, relative to SEQ ID NO: 248, or any fragment thereof,
or wherein the Cas protein is encoded by a polynucleotide comprising the sequence of SEQ ID NO: 248.

8. The Cas protein of claim 1, wherein the Cas protein comprises an amino acid sequence having one, two, three, four, five or more changes selected from amino acid substitutions, insertions, or deletions, relative to SEQ ID NO: 249, or any fragment thereof,
or wherein the Cas protein comprises the amino acid sequence of SEQ ID NO: 249,
or wherein the Cas protein is encoded by a polynucleotide comprising a sequence having at least 80%, 85%, 90%, 95%, or 98% or greater identity to SEQ ID NO: 250, or any fragment thereof,
or wherein the Cas protein is encoded by a polynucleotide comprising a sequence having one, two, three, four, five or more changes selected from nucleotide substitutions, insertions, or deletions, relative to SEQ ID NO: 250, or any fragment thereof,
or wherein the Cas protein is encoded by a polynucleotide comprising the sequence of SEQ ID NO: 250.

9. The Cas protein of claim 1, wherein the Cas protein comprises an amino acid sequence having one, two, three, four, five or more changes selected from amino acid substitutions, insertions, or deletions, relative to SEQ ID NO: 251, or any fragment thereof,
or wherein the Cas protein comprises the amino acid sequence of SEQ ID NO: 251,
or wherein the Cas protein is encoded by a polynucleotide comprising a sequence having at least 80%, 85%, 90%, 95%, or 98% or greater identity to SEQ ID NO: 252, or any fragment thereof,
or wherein the Cas protein is encoded by a polynucleotide comprising a sequence having one, two, three, four, five or more changes selected from nucleotide substitutions, insertions, or deletions, relative to SEQ ID NO: 252, or any fragment thereof,
or wherein the Cas protein is encoded by a polynucleotide comprising the sequence of SEQ ID NO: 252.

10. The Cas protein of claim 1, wherein the Cas protein comprises an amino acid sequence having one, two, three, four, five or more changes selected from amino acid substitutions, insertions, or deletions, relative to SEQ ID NO: 235, or any fragment thereof,
or wherein the Cas protein comprises the amino acid sequence of SEQ ID NO: 235,
or wherein the Cas protein is encoded by a polynucleotide comprising a sequence having at least 80%, 85%, 90%, 95%, or 98% or greater identity to SEQ ID NO: 236, or any fragment thereof,
or wherein the Cas protein is encoded by a polynucleotide comprising a sequence having one, two, three, four, five or more changes selected from nucleotide substitutions, insertions, or deletions, relative to SEQ ID NO: 236, or any fragment thereof,
or wherein the Cas protein is encoded by a polynucleotide comprising the sequence of SEQ ID NO: 236.

11. The Cas protein of claim any one of claims 1-10, wherein the Cas protein comprises at least one amino acid mutation that knocks out nuclease activity of the Cas protein.

12. The Cas protein of claim 11, wherein the at least one amino acid mutation is at least one of D10A, H600A, H845A, H599A, H840A, H604A, H839A, and D9A.

13. The Cas protein of any one of claims 11-12, wherein the Cas protein comprises an amino acid sequence having at least 80%, 85%, 90%, 95%, or 98% or greater identity to at least one of SEQ ID NOs: 59, 193, 197, 201, 205, 209, 213, 237, 225, or any fragment thereof.

14. The Cas protein of claim 13, wherein the Cas protein comprises an amino acid sequence having one, two, three, four, five or more changes selected from amino acid substitutions, insertions, or deletions, relative to at least one of SEQ ID NOs: 59, 193, 197, 201, 205, 209, 213, 237, 225, or any fragment thereof.

15. The Cas protein of claim 13 or 14, wherein the Cas protein comprises the amino acid sequence of at least one of SEQ ID NOs: 59, 193, 197, 201, 205, 209, 213, 237, or 225.

16. The Cas protein of any one of claims 11-15, wherein the Cas protein is encoded by a polynucleotide comprising a sequence having at least 80%, 85%, 90%, 95%, or 98% or greater identity to at least one of SEQ ID NOs: 60, 194, 198, 202, 206, 210, 214, 238, 226, or any fragment thereof.

17. The Cas protein of claim 16, wherein the Cas protein is encoded by a polynucleotide comprising a sequence having one, two, three, four, five or more changes selected from nucleotide substitutions, insertions, or deletions, relative to at least one of SEQ ID NOs: 60, 194, 198, 202, 206, 210, 214, 238, 226, or any fragment thereof.

18. The Cas protein of claim 16 or 17, wherein the Cas protein is encoded by a polynucleotide comprising the sequence of at least one of SEQ ID NOs: 60, 194, 198, 202, 206, 210, 214, 238, or 226.

19. The Cas protein of any one of claims 1-18, wherein the Cas protein recognizes a PAM sequence of AATA (SEQ ID NO: 71), NNA(A/G)TAN (SEQ ID NO: 273), NNAATA (SEQ ID NO: 274), NNG(T/C)(G/A)AN (SEQ ID NO: 275), NNGTAAA (SEQ ID NO: 276), NNGGNNN (SEQ ID NO: 277), NGG (SEQ ID NO: 2), NNAAAAN (SEQ ID NO: 278), NNAAAAA (SEQ ID NO: 279), NNGGNTN (SEQ ID NO: 280), NNAA(A/G)GN (SEQ ID NO: 281), and/or NNAAAG (SEQ ID NO: 282).

20. A fusion protein comprising two heterologous polypeptide domains, wherein the first polypeptide domain comprises the Cas protein of any one of claims 1-19, and wherein the second polypeptide domain has an activity selected from the group consisting of transcription activation activity, transcription repression activity, transcription release factor activity, histone modification activity, nuclease activity, nucleic acid association activity, methylase activity, and demethylase activity, or a combination thereof.

21. The fusion protein of claim 20, wherein the second polypeptide domain comprises a polypeptide selected from VP16, VP64, p65, TET1, VPR, VPH, Rta, p300, p300 core, KRAB, MECP2, EED, ERD, Mad mSIN3 interaction domain (SID), or Mad-SID repressor domain, SID4× repressor, Mxil repressor, SUV39H1, SUV39H2, G9A, ESET/SETBD1, Cir4, Su (var) 3-9, Pr-SET7/8, SUV4-20H1, PR-set7, Suv4-20, Set9, EZH2, RIZ1, JMJD2A/JHDM3A, JMJD2B, JMJ2D2C/GASC1, JMJD2D, Rph1, JARID1A/RBP2, JARID1B/PLU-1, JARID1C/SMCX, JARID1D/SMCY, Lid, Jhn2, Jmj2, HDAC1, HDAC2, HDAC3, HDAC8, Rpd3, Hos1, Cir6, HDAC4, HDAC5, HDAC7, HDAC9, Hda1, Cir3, SIRT1, SIRT2, Sir2, Hst1, Hst2, Hst3, Hst4, HDAC11, DNMT1, DNMT3a/3b, DNMT3A-3L, MET1, DRM3, ZMET2, CMT1, CMT2, Laminin A, Laminin B, CTCF, a domain having TATA box binding protein activity, ERF1, and ERF3.

22. The fusion protein of any one of claims 20-21, wherein the second polypeptide domain has transcription repression activity.

23. The fusion protein of claim 22, wherein the second polypeptide domain comprises KRAB.

24. The fusion protein of claim 23, wherein the KRAB comprises an amino acid sequence having at least 80%, 85%, 90%, 95%, or 98% or greater identity to SEQ ID NO: 45, or comprises an amino acid sequence having one, two, three, four, five or more changes selected from amino acid substitutions, insertions, or deletions, relative to SEQ ID NO: 45, or comprises the amino acid sequence of SEQ ID NO: 45, or is encoded by a polynucleotide comprising a sequence having at least 80%, 85%, 90%, 95%, or 98% or greater identity to SEQ ID NO: 46, or is encoded by a polynucleotide comprising a sequence having one, two, three, four, five or more changes selected from nucleotide substitutions, insertions, or deletions, relative to SEQ ID NO: 46 or is encoded by a polynucleotide comprising the sequence of SEQ ID NO: 46, or any fragment thereof.

25. The fusion protein of any one of claims 20-24, wherein the fusion protein comprises an amino acid sequence having at least 80%, 85%, 90%, 95%, or 98% or greater identity to at least one of SEQ ID NOs: 61, 217, 218, 219, 220, 221, 222, 239, 227, or comprises an amino acid sequence having one, two, three, four, five or more changes selected from amino acid substitutions, insertions, or deletions, relative to at least one of SEQ ID NOs: 61, 217, 218, 219, 220, 221, 222, 239, 227, or comprises the amino acid sequence of at least one of SEQ ID NOs: 61, 217, 218, 219, 220, 221, 222, 239, 227, or is encoded by a polynucleotide comprising a sequence having at least 80%, 85%, 90%, 95%, or 98% or greater identity to SEQ ID NO: 62 or 240 or 228, or is encoded by a polynucleotide comprising a sequence having one, two, three, four, five or more changes selected from nucleotide substitutions, insertions, or deletions, relative to SEQ ID NO: 62 or 240 or 228, or is encoded by a polynucleotide comprising the sequence of SEQ ID NO: 62 or 240 or 228, or any fragment thereof.

26. The fusion protein of any one of claims 20-21, wherein the second polypeptide domain has transcription activation activity.

27. The fusion protein of claim 26, wherein the second polypeptide domain comprises p300 or a fragment thereof or VP64 or a fragment thereof.

28. The fusion protein of claim 27, wherein the p300 or a fragment thereof comprises an amino acid sequence having at least 80%, 85%, 90%, 95%, or 98% or greater identity to SEQ ID NO: 41 or 42, or comprises an amino acid sequence having one, two, three, four, five or more changes selected from amino acid substitutions, insertions, or deletions, relative to SEQ ID NO: 41 or 42, or comprises the amino acid sequence of SEQ ID NO: 41 or 42, or any fragment thereof.

29. The fusion protein of any one of claims 20-24, wherein the fusion protein comprises an amino acid sequence having at least 80%, 85%, 90%, 95%, or 98% or greater identity to at least one of SEQ ID NOs: 253, 259, 263, 265, 267, 261, 269, 271, or 229, or comprises an amino acid sequence having one, two, three, four, five or more changes selected from amino acid substitutions, insertions, or deletions, relative to at least one of SEQ ID NOs: 253, 259, 263, 265, 267, 261, 269, 271, or 229, or comprises the amino acid sequence of at least one of SEQ ID NOs: 253, 259, 263, 265, 267, 261, 269, 271, or 229, or is encoded by a polynucleotide comprising a sequence having at least 80%, 85%, 90%, 95%, or 98% or greater identity to at least one of SEQ ID NO: 254, 260, 264, 266, 268, 262, 270, 272, or 230, or is encoded by a polynucleotide comprising a sequence having one, two, three, four, five or more changes selected from nucleotide substitutions, insertions, or deletions, relative to at least one of SEQ ID NO: 254, 260, 264, 266, 268, 262, 270, 272, or 230, or is encoded by a polynucleotide comprising the sequence of at least one of SEQ ID NO: 254, 260, 264, 266, 268, 262, 270, 272, or 230, or any fragment thereof.

30. A DNA targeting composition comprising:
the Cas protein of any one of claims 1-19 or the fusion protein of any one of claims 20-29; and
at least one guide RNA (gRNA) that targets the Cas protein to a target region of a target gene.

31. The DNA targeting composition of claim 30, wherein the gRNA targets the Cas protein to target region selected from a non-open chromatin region, an open chromatin region, a transcribed region of the target gene, a region upstream of a transcription start site of the target gene, a regulatory element of the target gene, an intron of the target gene, or an exon of the target gene.

32. The DNA targeting composition of claim 31, wherein the gRNA targets the Cas protein to a promoter of the target gene.

33. The DNA targeting composition of claim 31, wherein the target region is located between about 1 to about 1000 base pairs upstream of a transcription start site of the target gene.

34. The DNA targeting composition of any one of claims 30-33, wherein the DNA targeting composition comprises two or more gRNAs, each gRNA binding to a different target region.

35. The DNA targeting composition of any one of claims 30-34, wherein the at least one gRNA comprises the sequence of SEQ ID NO: 69 or 67 or is encoded by or targets a sequence comprising SEQ ID NO: 70 or 68.

36. The DNA targeting composition of any one of claims 30-34, wherein the at least one gRNA comprises a sequence selected from SEQ ID NOs: 195, 199, 203, 207, 211, 215, or is encoded by or targets a polynucleotide comprising a sequence selected from SEQ ID NOs: 196, 200, 204, 208, 212, 216.

37. The DNA targeting composition of any one of claims 30-36, wherein the at least one gRNA comprises a sequence selected from SEQ ID NOs: 91-94, 100-103, 108-122, 158-192, or is encoded by or targets a polynucleotide comprising a sequence selected from SEQ ID NOs: 76-90, 96-99, 123-157.

38. An isolated polynucleotide sequence encoding the Cas protein of any one of claims 1-19 or the fusion protein of any one of claims 20-29, or the DNA targeting composition of any one of claims 31-38.

39. A vector comprising: the isolated polynucleotide sequence of claim 38.

40. The vector of claim 39, wherein the vector is an adeno-associated virus (AAV) vector.

41. A cell comprising: the DNA targeting composition of any one of claims 30-37, or the isolated polynucleotide sequence of claim 38, or the vector of claim 39 or 40, or a combination thereof.

42. A pharmaceutical composition comprising: the DNA targeting composition of any one of claims 30-37, or the isolated polynucleotide sequence of claim 38, or the vector of claim 39 or 40, or a combination thereof.

43. A method of modulating expression of a gene in a cell or in a subject, the method comprising administering to the cell or the subject the DNA targeting composition of any one of claims 30-37, or the isolated polynucleotide sequence of claim 38, or the vector of claim 39 or 40, or the pharmaceutical composition of claim 42, or a combination thereof.

44. The method of claim 43, wherein the expression of the gene is increased relative to a control.

45. The method of claim 43, wherein the expression of the gene is decreased relative to a control.

46. The method of claim 43, wherein the gene comprises the dystrophin gene.

47. A method of correcting a mutant gene in a cell, the method comprising administering to the cell or the subject the DNA targeting composition of any one of claims 30-37, or the isolated polynucleotide sequence of claim 38, or the vector of claim 39 or 40, or the pharmaceutical composition of claim 42, or a combination thereof.

48. The method of claim 47, further comprising administering to the cell or subject a donor DNA.

49. The method of claim 47 or 48, wherein correcting a mutant gene comprises deleting, rearranging, or replacing the mutant gene.

50. The method of any one of claims 7-49, wherein the gene comprises the dystrophin gene.

51. A method of treating a disease in a subject, the method comprising administering to the subject the DNA targeting composition of any one of claims 30-37, or the isolated polynucleotide sequence of claim 38, or the vector of claim 39 or 40, or the cell of claim 41, or the pharmaceutical composition of claim 42, or a combination thereof.

52. The method of claim 51, wherein the DNA targeting composition, or the isolated polynucleotide sequence, or the vector, or the cell, or the pharmaceutical composition, or a combination thereof, is administered to skeletal muscle or cardiac muscle of the subject.

53. The method of claim 51 or 52, wherein the disease comprises Duchenne muscular dystrophy (DMD) or Becker muscular dystrophy (BMD).


SEQUENCE LISTING (307 sequences)

SEQ ID NO: 1-17 - skipped (17 entries)

SEQ ID NO: 18 - DNA, 86 bp, synthetic construct
  source 1..86: mol_type = other DNA
gtttaagagctatgctggaaacagcatagcaagtttaaataaggctagtccgttatcaacttgaaaaagtggcaccgagtcggtgc

SEQ ID NO: 19 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 20 - protein, 7 aa, synthetic construct
PKKKRKV

SEQ ID NO: 21 - protein, 5 aa, synthetic construct
  REPEAT 1..5: note = repeat unit:0 to 10 times
GGGGS

SEQ ID NO: 22 - protein, 5 aa, synthetic construct
GGGGG

SEQ ID NO: 23 - protein, 5 aa, synthetic construct
GGAGG

SEQ ID NO: 24 - protein, 7 aa, synthetic construct
GGGGSSS

SEQ ID NO: 25 - protein, 7 aa, synthetic construct
GGGGAAA

SEQ ID NO: 26 - protein, 1368 aa, Streptococcus pyogenes [sequence omitted]

SEQ ID NO: 27 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 29 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - DNA, 3159 bp, synthetic construct
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 31 - DNA, 3159 bp, synthetic construct
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 4107 bp, synthetic construct
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 33 - DNA, 3158 bp, synthetic construct
  source 1..3158: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 34 - DNA, 3159 bp, synthetic construct
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 35 - DNA, 3159 bp, synthetic construct
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 36 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 37 - DNA, 3242 bp, synthetic construct
  source 1..3242: mol_type = other DNA
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 38 - DNA, 3255 bp, synthetic construct
  source 1..3255: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 39 - DNA, 3156 bp, synthetic construct
  source 1..3156: mol_type = other DNA
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 40 - DNA, 7009 bp, synthetic construct
  source 1..7009: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 41 - protein, 2414 aa, synthetic construct
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 42 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 43 - protein, 1497 aa, synthetic construct
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, synthetic construct
  source 1..4491: mol_type = other DNA
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 65 aa, synthetic construct
RTLVTFKDVFVDFTREEWKLLDTAQQILYRNVMLENYKNLVSLGYQLTKPDVILRLEKGEEPWLV

SEQ ID NO: 46 - DNA, 195 bp, synthetic construct
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 47 - protein, 1519 aa, synthetic construct
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 48 - DNA, 4560 bp, synthetic construct
  source 1..4560: mol_type = other DNA
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 49 - protein, 1190 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 50 - DNA, 3570 bp, synthetic construct
  source 1..3570: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 51 - protein, 718 aa, synthetic construct
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 52 - DNA, 2154 bp, synthetic construct
  source 1..2154: mol_type = other DNA
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 53 - protein, 378 aa, synthetic construct
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 54 - DNA, 1134 bp, synthetic construct
  source 1..1134: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctagggtcactacccagcgccagcgtcgagttcgaaggcagcggcgggccttcagggcagatcagcaaccaggccctggctctggcccctagctccgctccagtgctggcccagactatggtgccctctagtgctatggtgcctctggcccagccacctgctccagcccctgtgctgaccccaggaccaccccagtcactgagcgccccagtgcccaagtctacacaggccggcgaggggactctgagtgaagctctgctgcacctgcagttcgacgctgatgaggacctgggagctctgctggggaacagcaccgatcccggagtgttcacagatctggcctccgtggacaactctgagtttcagcagctgctgaatcagggcgtgtccatgtctcatagtacagccgaaccaatgctgatggagtaccccgaagccattacccggctggtgaccggcagccagcggccccccgaccccgctccaactcccctgggaaccagcggcctgcctaatgggctgtccggagatgaagacttctcaagcatcgctgatatggactttagtgccctgctgtcacagatttcctctagtgggcagggaggaggtggaagcggcttcagcgtggacaccagtgccctgctggacctgttcagcccctcggtgaccgtgcccgacatgagcctgcctgaccttgacagcagcctggccagtatccaagagctcctgtctccccaggagccccccaggcctcccgaggcagagaacagcagcccggattcagggaagcagctggtgcactacacagcgcagccgctgttcctgctggaccccggctccgtggacaccgggagcaacgacctgccggtgctgtttgagctgggagagggctcctacttctccgaaggggacggcttcgccgaggaccccaccatctccctgctgacaggctcggagcctcccaaagccaaggaccccactgtctcc

SEQ ID NO: 55 - protein, 522 aa, synthetic construct
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - DNA, 1566 bp, synthetic construct
  source 1..1566: mol_type = other DNA
gatgctttagacgattttgacttagatatgcttggttcagacgcgttagacgacttcgacctagacatgttaggctcagatgcattggacgacttcgatttagatatgttgggctccgatgccctagatgactttgatctagatatgctaggtagtcccaaaaagaagaggaaagtgggatcccagtatctgcccgacacagatgatagacaccgaatcgaagagaaacgcaagcgaacgtatgaaaccttcaaatcgatcatgaagaaatcgcccttctcgggtccgaccgatcccaggcccccaccgagaaggattgcggtcccgtcccgctcgtcggccagcgtgccgaagcctgcgccgcagccctaccccttcacgtcgagcctgagcacaatcaattatgacgagttcccgacgatggtgttcccctcgggacaaatctcacaagcctcggcgctcgcaccagcgcctccccaagtccttccgcaagcgcctgccccagcgcctgcaccggcaatggtgtccgccctcgcacaggcccctgcgcccgtccccgtgctcgcgcctggaccgccccaggcggtcgctccaccggctccgaagccgacgcaggccggagagggaacactctccgaagcacttcttcaactccagtttgatgacgaggatcttggagcactccttggaaactcgacagaccctgcggtgtttaccgacctcgcgtcagtagataactccgaatttcagcagcttttgaaccagggtatcccggtcgcgccacatacaacggagcccatgttgatggaataccccgaagcaatcacgagacttgtgacgggagcgcagcggcctcccgatcccgcacccgcacctttgggggcacctggcctccctaacggacttttgagcggcgacgaggatttctcctccatcgccgatatggatttctcagccttgctgtcacagatttccagcggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 57 - protein, 1122 aa, Streptococcus uberis
MTNGMILGLDIGVASVGVGIIEADSGKVIHASSRIFPSANADNNVERRKFRGSRRLLRRKKHRVKRLQDLFDKYDIVTNFDNLNLNPYELRVKGLNEPLSNEELFASLRNITKHRGISYLDDAEDDSSGNGTEYAKAIELNQQLLKEKTPGQIQYDRLNQYGQLRGNFDIVDENGEIHHVINVFSTSSYRKEAEQILKKQSETNTSISTDFINDFIQLLTSKRKYYHGPGNPKSRTDYGRYRTDGTDLDNIFDVLIGKCSFYPEEYRASKTSYTAQEFNFLNDLNNLTLPTETGKLSEQQKIDLVNWAKETKILGPKKLLQEIAKRNNCKYEDIRGYRLDKKDNPDMHVFDVYRKMNFDLETISVKDLSVDSLNQLARILTLNTEREGIEEAIKNLMPNQFTEKQMLELIAFRKSNSSIFGKGWHSLSIKLMKELIPELYHTSDEQMTILNRFGKFKLTKLDSKRTNYIDENFVTDEIYNPVVAKSVRQAIKIINASIKKWGDFDKIVIEMPRDKNEEEERKRIADGQKVNAKEKEQAEKHAAKLFNGKEELPSEVFHGYKELALRIRLWYQQDQKCLYSGKEITISDLIYNRELFEIDHILPLSLSFDDSLSNKVLVYRWANQEKGQRTPFQALDSMKSAWSYREFKNAILHNSKISRRKRDYFLTEQDISKIEVKQKFIERNLVDTRYASRTVLNVLQQSLKNLEKETKVSVVRGQFTSQLRRKWHIDKTRDTYHHHAVDALIIAASAKLRYWKKQGDILFENYLINRHVDRVTGEIQSDDSYKEEVFTPPYDGFVQTISNPGFEDEILFSYQVDSKVNRKISDATIYATRSAKLEKDKKEQTYVLGKIKDIYSQTGFENFLKIYNKDKSKFLIYQKDPETWEKIIEPILKNYREFDNKGKDIVNPFEKYRNDNGPICKYSRKGNGPEIKQFKYYDTVYKITSGLDISPRESRNKVILQSLNPWRTDFYFNPKTMKYELMGIRYVDLEFEKGTGDYLISDNLYKEIKKNEGISELSVFKFTLYKNDLLLIKDTENNEEQIFRFWSRNDLSSKNRVELKPYDRSRFSGNEILITKMGKAPKQCIKTLTYQNISIYKIKTDILGFKYYLKNEGNKPLLHFKK

SEQ ID NO: 58 - DNA, 3366 bp, synthetic construct
  source 1..3366: mol_type = other DNA
atgactaatgggatgattctgggcttagatatcggagtcgcgtctgtaggggtaggaattatcgaggccgatagtggcaaggtaattcacgcaagctcacggatcttccctagtgctaatgccgacaataatgtggagcgccgcaagttccggggatctaggcgccttcttaggaggaaaaagcacagggttaagcgcttacaggatctgtttgacaagtacgatatcgtgactaacttcgataacctcaaccttaacccctacgagctgcgagttaagggcttaaacgagccattgagcaacgaagagctcttcgcatcactccggaacatcacaaagcacaggggcatttcctatctcgatgatgctgaggatgactcttctggaaacgggacagaatacgccaaagcgatagagctgaatcagcagcttctgaaagaaaagacccccggtcagatccagtacgacagactcaatcagtatgggcaactgagaggcaatttcgatatcgtggatgaaaacggcgagattcaccacgtgataaacgttttttcaacatcaagttacagaaaggaagccgagcagattctcaagaagcagtctgaaacgaatactagtatcagcaccgactttataaatgatttcatccaattgctgacctctaagaggaaatattaccatggtcctggtaatccaaagagccgcacagattacgggcgctaccggacggatgggacggatctcgataacatcttcgatgttctgataggtaaatgcagcttttacccagaggagtaccgagccagcaagacgagctacactgcccaagagttcaactttcttaatgacttgaataacctgaccttaccaaccgagacaggcaagttgagcgagcagcagaagatcgacctggtgaattgggctaaggagacaaagatcctcggaccgaaaaagctgcttcaggaaattgccaagaggaacaactgcaagtacgaggacattcgcggctatcggcttgataagaaagataaccccgatatgcatgtatttgatgtgtatcggaagatgaattttgacctggagactatttccgttaaggatctgtcagtcgactctctgaatcagctcgcgcgaattctgacactgaacaccgagagggaggggatcgaagaggccatcaaaaatctgatgccaaaccagttcaccgagaagcaaatgcttgaactcatcgccttccgcaagagtaattcctctatctttgggaaggggtggcacagtctgtcaattaaactgatgaaagagctgatacccgagctctaccacaccagtgacgaacaaatgaccatactcaatcgatttggtaagttcaagctcacgaagctcgactcaaaaaggaccaattacatcgatgaaaactttgtcactgatgaaatctataaccctgtagtggccaagagtgtgaggcaggcaataaagatcatcaacgcttccattaaaaagtggggggactttgataagatcgtgattgagatgccacgcgacaagaatgaggaggaggaaaggaaacgaatcgccgatggccagaaggtgaatgctaaggaaaaagagcaggccgagaagcacgccgcaaagctctttaatggcaaggaagagctcccttctgaagttttccatggatataaggagctggctttgcgaattagactctggtatcagcaagaccagaagtgcctctattctggcaaggagataacaatttcagacctgatctacaacagggagctctttgagattgaccatatccttccgctgtctctttcttttgacgacagtctgtctaacaaggtcctggtttacagatgggcaaatcaggagaagggccagaggacccctttccaagcccttgattccatgaaatcagcgtggtcctatcgggagttcaagaatgcaatcctgcacaattctaaaatcagccggagaaagcgtgactattttctgacagaacaagacattagtaagattgaggtgaaacaaaagtttattgagaggaacttggtggacacacggtacgccagtagaacagttctcaacgtgctgcagcagtccctgaagaatctggagaaggagactaaggtgtccgttgtccgaggacagttcacgtcccagctgcgccggaaatggcacatagataagaccagggatacttaccatcaccatgcggtggacgcactgattatcgcggcctccgctaagttgagatattggaagaaacagggcgacatcttgttcgagaactatctcatcaatcgccacgtagatagagtaaccggggagatacaatctgacgatagctataaggaggaggtgttcacacctccctacgacggatttgtccagactattagcaacccagggtttgaggacgagatccttttctcctatcaggtagacagtaaagtcaacagaaagatctcagacgccacgatatacgctacgaggtctgcgaagctcgagaaggacaagaaggaacagacgtatgtcttgggtaagataaaagatatctattcacaaactggttttgagaacttcctgaagatctataataaggacaagagtaagttcctgatctaccagaaggaccctgagacttgggaaaagatcattgaaccaattctcaaaaattatcgggaattcgataataaaggcaaggatatcgtgaatccatttgagaaatacaggaatgataacgggcctatctgcaagtacagtcggaaaggcaacggccctgagatcaaacaatttaaatactacgacaccgtttacaaaattacaagcggtctcgacatcagcccccgcgaatcaagaaataaggtaattcttcaaagcctgaatccgtggagaaccgacttctactttaaccctaagactatgaagtacgaacttatgggtatcagatatgtcgacctggagttcgagaaaggaacaggggactacctgatttctgacaatctctataaagagattaaaaagaacgaggggatctctgagctgagtgtattcaagttcacactctacaagaacgatctcctgctgatcaaggacactgagaacaacgaagagcaaatttttaggttttggtctcggaatgacctgtcctccaaaaaccgggtggaactgaagccctacgataggtcccgcttttccggcaatgagatccttatcaccaaaatgggcaaggcacctaagcaatgcattaagactttaacataccaaaacatctccatttataaaatcaaaacagacatcctgggattcaaatactatctgaaaaacgaaggaaataagccattactgcactttaagaag

SEQ ID NO: 59 - protein, 1122 aa, synthetic construct
MTNGMILGLAIGVASVGVGIIEADSGKVIHASSRIFPSANADNNVERRKFRGSRRLLRRKKHRVKRLQDLFDKYDIVTNFDNLNLNPYELRVKGLNEPLSNEELFASLRNITKHRGISYLDDAEDDSSGNGTEYAKAIELNQQLLKEKTPGQIQYDRLNQYGQLRGNFDIVDENGEIHHVINVFSTSSYRKEAEQILKKQSETNTSISTDFINDFIQLLTSKRKYYHGPGNPKSRTDYGRYRTDGTDLDNIFDVLIGKCSFYPEEYRASKTSYTAQEFNFLNDLNNLTLPTETGKLSEQQKIDLVNWAKETKILGPKKLLQEIAKRNNCKYEDIRGYRLDKKDNPDMHVFDVYRKMNFDLETISVKDLSVDSLNQLARILTLNTEREGIEEAIKNLMPNQFTEKQMLELIAFRKSNSSIFGKGWHSLSIKLMKELIPELYHTSDEQMTILNRFGKFKLTKLDSKRTNYIDENFVTDEIYNPVVAKSVRQAIKIINASIKKWGDFDKIVIEMPRDKNEEEERKRIADGQKVNAKEKEQAEKHAAKLFNGKEELPSEVFHGYKELALRIRLWYQQDQKCLYSGKEITISDLIYNRELFEIDAILPLSLSFDDSLSNKVLVYRWANQEKGQRTPFQALDSMKSAWSYREFKNAILHNSKISRRKRDYFLTEQDISKIEVKQKFIERNLVDTRYASRTVLNVLQQSLKNLEKETKVSVVRGQFTSQLRRKWHIDKTRDTYHHHAVDALIIAASAKLRYWKKQGDILFENYLINRHVDRVTGEIQSDDSYKEEVFTPPYDGFVQTISNPGFEDEILFSYQVDSKVNRKISDATIYATRSAKLEKDKKEQTYVLGKIKDIYSQTGFENFLKIYNKDKSKFLIYQKDPETWEKIIEPILKNYREFDNKGKDIVNPFEKYRNDNGPICKYSRKGNGPEIKQFKYYDTVYKITSGLDISPRESRNKVILQSLNPWRTDFYFNPKTMKYELMGIRYVDLEFEKGTGDYLISDNLYKEIKKNEGISELSVFKFTLYKNDLLLIKDTENNEEQIFRFWSRNDLSSKNRVELKPYDRSRFSGNEILITKMGKAPKQCIKTLTYQNISIYKIKTDILGFKYYLKNEGNKPLLHFKK

SEQ ID NO: 60 - DNA, 3366 bp, synthetic construct
  source 1..3366: mol_type = other DNA
atgactaatgggatgattctgggcttagcaatcggagtcgcgtctgtaggggtaggaattatcgaggccgatagtggcaaggtaattcacgcaagctcacggatcttccctagtgctaatgccgacaataatgtggagcgccgcaagttccggggatctaggcgccttcttaggaggaaaaagcacagggttaagcgcttacaggatctgtttgacaagtacgatatcgtgactaacttcgataacctcaaccttaacccctacgagctgcgagttaagggcttaaacgagccattgagcaacgaagagctcttcgcatcactccggaacatcacaaagcacaggggcatttcctatctcgatgatgctgaggatgactcttctggaaacgggacagaatacgccaaagcgatagagctgaatcagcagcttctgaaagaaaagacccccggtcagatccagtacgacagactcaatcagtatgggcaactgagaggcaatttcgatatcgtggatgaaaacggcgagattcaccacgtgataaacgttttttcaacatcaagttacagaaaggaagccgagcagattctcaagaagcagtctgaaacgaatactagtatcagcaccgactttataaatgatttcatccaattgctgacctctaagaggaaatattaccatggtcctggtaatccaaagagccgcacagattacgggcgctaccggacggatgggacggatctcgataacatcttcgatgttctgataggtaaatgcagcttttacccagaggagtaccgagccagcaagacgagctacactgcccaagagttcaactttcttaatgacttgaataacctgaccttaccaaccgagacaggcaagttgagcgagcagcagaagatcgacctggtgaattgggctaaggagacaaagatcctcggaccgaaaaagctgcttcaggaaattgccaagaggaacaactgcaagtacgaggacattcgcggctatcggcttgataagaaagataaccccgatatgcatgtatttgatgtgtatcggaagatgaattttgacctggagactatttccgttaaggatctgtcagtcgactctctgaatcagctcgcgcgaattctgacactgaacaccgagagggaggggatcgaagaggccatcaaaaatctgatgccaaaccagttcaccgagaagcaaatgcttgaactcatcgccttccgcaagagtaattcctctatctttgggaaggggtggcacagtctgtcaattaaactgatgaaagagctgatacccgagctctaccacaccagtgacgaacaaatgaccatactcaatcgatttggtaagttcaagctcacgaagctcgactcaaaaaggaccaattacatcgatgaaaactttgtcactgatgaaatctataaccctgtagtggccaagagtgtgaggcaggcaataaagatcatcaacgcttccattaaaaagtggggggactttgataagatcgtgattgagatgccacgcgacaagaatgaggaggaggaaaggaaacgaatcgccgatggccagaaggtgaatgctaaggaaaaagagcaggccgagaagcacgccgcaaagctctttaatggcaaggaagagctcccttctgaagttttccatggatataaggagctggctttgcgaattagactctggtatcagcaagaccagaagtgcctctattctggcaaggagataacaatttcagacctgatctacaacagggagctctttgagattgacgccatccttccgctgtctctttcttttgacgacagtctgtctaacaaggtcctggtttacagatgggcaaatcaggagaagggccagaggacccctttccaagcccttgattccatgaaatcagcgtggtcctatcgggagttcaagaatgcaatcctgcacaattctaaaatcagccggagaaagcgtgactattttctgacagaacaagacattagtaagattgaggtgaaacaaaagtttattgagaggaacttggtggacacacggtacgccagtagaacagttctcaacgtgctgcagcagtccctgaagaatctggagaaggagactaaggtgtccgttgtccgaggacagttcacgtcccagctgcgccggaaatggcacatagataagaccagggatacttaccatcaccatgcggtggacgcactgattatcgcggcctccgctaagttgagatattggaagaaacagggcgacatcttgttcgagaactatctcatcaatcgccacgtagatagagtaaccggggagatacaatctgacgatagctataaggaggaggtgttcacacctccctacgacggatttgtccagactattagcaacccagggtttgaggacgagatccttttctcctatcaggtagacagtaaagtcaacagaaagatctcagacgccacgatatacgctacgaggtctgcgaagctcgagaaggacaagaaggaacagacgtatgtcttgggtaagataaaagatatctattcacaaactggttttgagaacttcctgaagatctataataaggacaagagtaagttcctgatctaccagaaggaccctgagacttgggaaaagatcattgaaccaattctcaaaaattatcgggaattcgataataaaggcaaggatatcgtgaatccatttgagaaatacaggaatgataacgggcctatctgcaagtacagtcggaaaggcaacggccctgagatcaaacaatttaaatactacgacaccgtttacaaaattacaagcggtctcgacatcagcccccgcgaatcaagaaataaggtaattcttcaaagcctgaatccgtggagaaccgacttctactttaaccctaagactatgaagtacgaacttatgggtatcagatatgtcgacctggagttcgagaaaggaacaggggactacctgatttctgacaatctctataaagagattaaaaagaacgaggggatctctgagctgagtgtattcaagttcacactctacaagaacgatctcctgctgatcaaggacactgagaacaacgaagagcaaatttttaggttttggtctcggaatgacctgtcctccaaaaaccgggtggaactgaagccctacgataggtcccgcttttccggcaatgagatccttatcaccaaaatgggcaaggcacctaagcaatgcattaagactttaacataccaaaacatctccatttataaaatcaaaacagacatcctgggattcaaatactatctgaaaaacgaaggaaataagccattactgcactttaagaag

SEQ ID NO: 61 - protein, 1205 aa, synthetic construct
MTNGMILGLAIGVASVGVGIIEADSGKVIHASSRIFPSANADNNVERRKFRGSRRLLRRKKHRVKRLQDLFDKYDIVTNFDNLNLNPYELRVKGLNEPLSNEELFASLRNITKHRGISYLDDAEDDSSGNGTEYAKAIELNQQLLKEKTPGQIQYDRLNQYGQLRGNFDIVDENGEIHHVINVFSTSSYRKEAEQILKKQSETNTSISTDFINDFIQLLTSKRKYYHGPGNPKSRTDYGRYRTDGTDLDNIFDVLIGKCSFYPEEYRASKTSYTAQEFNFLNDLNNLTLPTETGKLSEQQKIDLVNWAKETKILGPKKLLQEIAKRNNCKYEDIRGYRLDKKDNPDMHVFDVYRKMNFDLETISVKDLSVDSLNQLARILTLNTEREGIEEAIKNLMPNQFTEKQMLELIAFRKSNSSIFGKGWHSLSIKLMKELIPELYHTSDEQMTILNRFGKFKLTKLDSKRTNYIDENFVTDEIYNPVVAKSVRQAIKIINASIKKWGDFDKIVIEMPRDKNEEEERKRIADGQKVNAKEKEQAEKHAAKLFNGKEELPSEVFHGYKELALRIRLWYQQDQKCLYSGKEITISDLIYNRELFEIDAILPLSLSFDDSLSNKVLVYRWANQEKGQRTPFQALDSMKSAWSYREFKNAILHNSKISRRKRDYFLTEQDISKIEVKQKFIERNLVDTRYASRTVLNVLQQSLKNLEKETKVSVVRGQFTSQLRRKWHIDKTRDTYHHHAVDALIIAASAKLRYWKKQGDILFENYLINRHVDRVTGEIQSDDSYKEEVFTPPYDGFVQTISNPGFEDEILFSYQVDSKVNRKISDATIYATRSAKLEKDKKEQTYVLGKIKDIYSQTGFENFLKIYNKDKSKFLIYQKDPETWEKIIEPILKNYREFDNKGKDIVNPFEKYRNDNGPICKYSRKGNGPEIKQFKYYDTVYKITSGLDISPRESRNKVILQSLNPWRTDFYFNPKTMKYELMGIRYVDLEFEKGTGDYLISDNLYKEIKKNEGISELSVFKFTLYKNDLLLIKDTENNEEQIFRFWSRNDLSSKNRVELKPYDRSRFSGNEILITKMGKAPKQCIKTLTYQNISIYKIKTDILGFKYYLKNEGNKPLLHFKKTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 62 - DNA, 3615 bp, synthetic construct
  source 1..3615: mol_type = other DNA
atgactaatgggatgattctgggcttagcaatcggagtcgcgtctgtaggggtaggaattatcgaggccgatagtggcaaggtaattcacgcaagctcacggatcttccctagtgctaatgccgacaataatgtggagcgccgcaagttccggggatctaggcgccttcttaggaggaaaaagcacagggttaagcgcttacaggatctgtttgacaagtacgatatcgtgactaacttcgataacctcaaccttaacccctacgagctgcgagttaagggcttaaacgagccattgagcaacgaagagctcttcgcatcactccggaacatcacaaagcacaggggcatttcctatctcgatgatgctgaggatgactcttctggaaacgggacagaatacgccaaagcgatagagctgaatcagcagcttctgaaagaaaagacccccggtcagatccagtacgacagactcaatcagtatgggcaactgagaggcaatttcgatatcgtggatgaaaacggcgagattcaccacgtgataaacgttttttcaacatcaagttacagaaaggaagccgagcagattctcaagaagcagtctgaaacgaatactagtatcagcaccgactttataaatgatttcatccaattgctgacctctaagaggaaatattaccatggtcctggtaatccaaagagccgcacagattacgggcgctaccggacggatgggacggatctcgataacatcttcgatgttctgataggtaaatgcagcttttacccagaggagtaccgagccagcaagacgagctacactgcccaagagttcaactttcttaatgacttgaataacctgaccttaccaaccgagacaggcaagttgagcgagcagcagaagatcgacctggtgaattgggctaaggagacaaagatcctcggaccgaaaaagctgcttcaggaaattgccaagaggaacaactgcaagtacgaggacattcgcggctatcggcttgataagaaagataaccccgatatgcatgtatttgatgtgtatcggaagatgaattttgacctggagactatttccgttaaggatctgtcagtcgactctctgaatcagctcgcgcgaattctgacactgaacaccgagagggaggggatcgaagaggccatcaaaaatctgatgccaaaccagttcaccgagaagcaaatgcttgaactcatcgccttccgcaagagtaattcctctatctttgggaaggggtggcacagtctgtcaattaaactgatgaaagagctgatacccgagctctaccacaccagtgacgaacaaatgaccatactcaatcgatttggtaagttcaagctcacgaagctcgactcaaaaaggaccaattacatcgatgaaaactttgtcactgatgaaatctataaccctgtagtggccaagagtgtgaggcaggcaataaagatcatcaacgcttccattaaaaagtggggggactttgataagatcgtgattgagatgccacgcgacaagaatgaggaggaggaaaggaaacgaatcgccgatggccagaaggtgaatgctaaggaaaaagagcaggccgagaagcacgccgcaaagctctttaatggcaaggaagagctcccttctgaagttttccatggatataaggagctggctttgcgaattagactctggtatcagcaagaccagaagtgcctctattctggcaaggagataacaatttcagacctgatctacaacagggagctctttgagattgacgccatccttccgctgtctctttcttttgacgacagtctgtctaacaaggtcctggtttacagatgggcaaatcaggagaagggccagaggacccctttccaagcccttgattccatgaaatcagcgtggtcctatcgggagttcaagaatgcaatcctgcacaattctaaaatcagccggagaaagcgtgactattttctgacagaacaagacattagtaagattgaggtgaaacaaaagtttattgagaggaacttggtggacacacggtacgccagtagaacagttctcaacgtgctgcagcagtccctgaagaatctggagaaggagactaaggtgtccgttgtccgaggacagttcacgtcccagctgcgccggaaatggcacatagataagaccagggatacttaccatcaccatgcggtggacgcactgattatcgcggcctccgctaagttgagatattggaagaaacagggcgacatcttgttcgagaactatctcatcaatcgccacgtagatagagtaaccggggagatacaatctgacgatagctataaggaggaggtgttcacacctccctacgacggatttgtccagactattagcaacccagggtttgaggacgagatccttttctcctatcaggtagacagtaaagtcaacagaaagatctcagacgccacgatatacgctacgaggtctgcgaagctcgagaaggacaagaaggaacagacgtatgtcttgggtaagataaaagatatctattcacaaactggttttgagaacttcctgaagatctataataaggacaagagtaagttcctgatctaccagaaggaccctgagacttgggaaaagatcattgaaccaattctcaaaaattatcgggaattcgataataaaggcaaggatatcgtgaatccatttgagaaatacaggaatgataacgggcctatctgcaagtacagtcggaaaggcaacggccctgagatcaaacaatttaaatactacgacaccgtttacaaaattacaagcggtctcgacatcagcccccgcgaatcaagaaataaggtaattcttcaaagcctgaatccgtggagaaccgacttctactttaaccctaagactatgaagtacgaacttatgggtatcagatatgtcgacctggagttcgagaaaggaacaggggactacctgatttctgacaatctctataaagagattaaaaagaacgaggggatctctgagctgagtgtattcaagttcacactctacaagaacgatctcctgctgatcaaggacactgagaacaacgaagagcaaatttttaggttttggtctcggaatgacctgtcctccaaaaaccgggtggaactgaagccctacgataggtcccgcttttccggcaatgagatccttatcaccaaaatgggcaaggcacctaagcaatgcattaagactttaacataccaaaacatctccatttataaaatcaaaacagacatcctgggattcaaatactatctgaaaaacgaaggaaataagccattactgcactttaagaagaccggtcctaagaaaaagcggaaagtggctagcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 63 - protein, 1184 aa, synthetic construct
MGHHHHHHGGGSGMDYKDHDGDYKDHDIDYKDDDDKHVNPKKKRKVGRGTGMTNGMILGLDIGVASVGVGIIEADSGKVIHASSRIFPSANADNNVERRKFRGSRRLLRRKKHRVKRLQDLFDKYDIVTNFDNLNLNPYELRVKGLNEPLSNEELFASLRNITKHRGISYLDDAEDDSSGNGTEYAKAIELNQQLLKEKTPGQIQYDRLNQYGQLRGNFDIVDENGEIHHVINVFSTSSYRKEAEQILKKQSETNTSISTDFINDFIQLLTSKRKYYHGPGNPKSRTDYGRYRTDGTDLDNIFDVLIGKCSFYPEEYRASKTSYTAQEFNFLNDLNNLTLPTETGKLSEQQKIDLVNWAKETKILGPKKLLQEIAKRNNCKYEDIRGYRLDKKDNPDMHVFDVYRKMNFDLETISVKDLSVDSLNQLARILTLNTEREGIEEAIKNLMPNQFTEKQMLELIAFRKSNSSIFGKGWHSLSIKLMKELIPELYHTSDEQMTILNRFGKFKLTKLDSKRTNYIDENFVTDEIYNPVVAKSVRQAIKIINASIKKWGDFDKIVIEMPRDKNEEEERKRIADGQKVNAKEKEQAEKHAAKLFNGKEELPSEVFHGYKELALRIRLWYQQDQKCLYSGKEITISDLIYNRELFEIDHILPLSLSFDDSLSNKVLVYRWANQEKGQRTPFQALDSMKSAWSYREFKNAILHNSKISRRKRDYFLTEQDISKIEVKQKFIERNLVDTRYASRTVLNVLQQSLKNLEKETKVSVVRGQFTSQLRRKWHIDKTRDTYHHHAVDALIIAASAKLRYWKKQGDILFENYLINRHVDRVTGEIQSDDSYKEEVFTPPYDGFVQTISNPGFEDEILFSYQVDSKVNRKISDATIYATRSAKLEKDKKEQTYVLGKIKDIYSQTGFENFLKIYNKDKSKFLIYQKDPETWEKIIEPILKNYREFDNKGKDIVNPFEKYRNDNGPICKYSRKGNGPEIKQFKYYDTVYKITSGLDISPRESRNKVILQSLNPWRTDFYFNPKTMKYELMGIRYVDLEFEKGTGDYLISDNLYKEIKKNEGISELSVFKFTLYKNDLLLIKDTENNEEQIFRFWSRNDLSSKNRVELKPYDRSRFSGNEILITKMGKAPKQCIKTLTYQNISIYKIKTDILGFKYYLKNEGNKPLLHFKKTGPKKKRKVAV

SEQ ID NO: 64 - protein, 1384 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVMDKHHHHHH

SEQ ID NO: 65 - RNA, 36 nt, synthetic construct
  source 1..36: mol_type = other RNA
gtttttgtactctcaagatttaagtaactataaaac

SEQ ID NO: 66 - DNA, 36 bp, synthetic construct
  source 1..36: mol_type = other DNA
gtttttgtactctcaagatttaagtaactataaaac

SEQ ID NO: 67 - RNA, 88 nt, synthetic construct
  source 1..88: mol_type = other RNA
atagttacttaaatcttgctgagcctacaaagataaggcttcatgccgaattcaagcaccccatcattgatggggtgcttttcgtatt

SEQ ID NO: 68 - DNA, 88 bp, synthetic construct
  source 1..88: mol_type = other DNA
atagttacttaaatcttgctgagcctacaaagataaggcttcatgccgaattcaagcaccccatcattgatggggtgcttttcgtatt

SEQ ID NO: 69 - RNA, 103 nt, synthetic construct
  source 1..103: mol_type = other RNA
gtttttgtactctcaagatttcgaaaaatcttgctgagcctacaaagataaggcttcatgccgaattcaagcaccccatcattgatggggtgcttttcgtatt

SEQ ID NO: 70 - DNA, 103 bp, synthetic construct
  source 1..103: mol_type = other DNA
gtttttgtactctcaagatttcgaaaaatcttgctgagcctacaaagataaggcttcatgccgaattcaagcaccccatcattgatggggtgcttttcgtatt

SEQ ID NO: 71 - skipped

SEQ ID NO: 72 - DNA, 136 bp, synthetic construct
  source 1..136: mol_type = other DNA
aagctaatacgactcactatagggtgtcgtgatgcgtagacgggtttaagagctatgctggaaacagcatagcaagtttaaataaggctagtccgttatcaacttgaaaaagtggcaccgagtcggtgcttttttt

SEQ ID NO: 73 - DNA, 136 bp, synthetic construct
  source 1..136: mol_type = other DNA
aagctaatacgactcactataggcttcaagagcaacagtgctggtttaagagctatgctggaaacagcatagcaagtttaaataaggctagtccgttatcaacttgaaaaagtggcaccgagtcggtgcttttttt

SEQ ID NO: 74 - DNA, 146 bp, synthetic construct
  source 1..146: mol_type = other DNA
aagctaatacgactcactatagggtgtcgtgatgcgtagacgggtttttgtactctcaagatttcgaaaaatcttgctgagcctacaaagataaggcttcatgccgaattcaagcaccccatcattgatggggtgcttttcgtatt

SEQ ID NO: 75 - DNA, 146 bp, synthetic construct
  source 1..146: mol_type = other DNA
aagctaatacgactcactataggtgtttgagaatcaaaatcgggtttttgtactctcaagatttcgaaaaatcttgctgagcctacaaagataaggcttcatgccgaattcaagcaccccatcattgatggggtgcttttcgtatt

SEQ ID NO: 76 - DNA, 25 bp, synthetic construct
  source 1..25: mol_type = other DNA
gcatttgtttgagaatcaaaatcgg

SEQ ID NO: 77 - DNA, 24 bp, synthetic construct
  source 1..24: mol_type = other DNA
gatttgtttgagaatcaaaatcgg

SEQ ID NO: 78 - DNA, 22 bp, synthetic construct
  source 1..22: mol_type = other DNA
gttgtttgagaatcaaaatcgg

SEQ ID NO: 79 - DNA, 21 bp, synthetic construct
  source 1..21: mol_type = other DNA
gtgtttgagaatcaaaatcgg

SEQ ID NO: 80 - DNA, 19 bp, synthetic construct
  source 1..19: mol_type = other DNA
gtttgagaatcaaaatcgg

SEQ ID NO: 81 - DNA, 17 bp, synthetic construct
  source 1..17: mol_type = other DNA
gtgagaatcaaaatcgg

SEQ ID NO: 82 - DNA, 25 bp, synthetic construct
  source 1..25: mol_type = other DNA
gttctcaatgcatgggaatgaaggg

SEQ ID NO: 83 - DNA, 24 bp, synthetic construct
  source 1..24: mol_type = other DNA
gtctcaatgcatgggaatgaaggg

SEQ ID NO: 84 - DNA, 23 bp, synthetic construct
  source 1..23: mol_type = other DNA
gctcaatgcatgggaatgaaggg

SEQ ID NO: 85 - DNA, 22 bp, synthetic construct
  source 1..22: mol_type = other DNA
gtcaatgcatgggaatgaaggg

SEQ ID NO: 86 - DNA, 21 bp, synthetic construct
  source 1..21: mol_type = other DNA
gcaatgcatgggaatgaaggg

SEQ ID NO: 87 - DNA, 20 bp, synthetic construct
  source 1..20: mol_type = other DNA
gaatgcatgggaatgaaggg

SEQ ID NO: 88 - DNA, 19 bp, synthetic construct
  source 1..19: mol_type = other DNA
gatgcatgggaatgaaggg

SEQ ID NO: 89 - DNA, 18 bp, synthetic construct
  source 1..18: mol_type = other DNA
gtgcatgggaatgaaggg

SEQ ID NO: 90 - DNA, 17 bp, synthetic construct
  source 1..17: mol_type = other DNA
ggcatgggaatgaaggg

SEQ ID NO: 91 - RNA, 123 nt, synthetic construct
  source 1..123: mol_type = other RNA
tgtttgagaatcaaaatcgggtttttgtactctcaagatttcgaaaaatcttgctgagcctacaaagataaggcttcatgccgaattcaagcaccccatcattgatggggtgcttttcgtatt

SEQ ID NO: 92 - RNA, 123 nt, synthetic construct
  source 1..123: mol_type = other RNA
gtgtcgtgatgcgtagacgggtttttgtactctcaagatttcgaaaaatcttgctgagcctacaaagataaggcttcatgccgaattcaagcaccccatcattgatggggtgcttttcgtatt

SEQ ID NO: 93 - RNA, 113 nt, synthetic construct
  source 1..113: mol_type = other RNA
cttcaagagcaacagtgctggtttaagagctatgctggaaacagcatagcaagtttaaataaggctagtccgttatcaacttgaaaaagtggcaccgagtcggtgcttttttt

SEQ ID NO: 94 - RNA, 113 nt, synthetic construct
  source 1..113: mol_type = other RNA
gtgtcgtgatgcgtagacgggtttaagagctatgctggaaacagcatagcaagtttaaataaggctagtccgttatcaacttgaaaaagtggcaccgagtcggtgcttttttt

SEQ ID NO: 95 - DNA, 398 bp, synthetic construct
  source 1..398: mol_type = other DNA
gtctcgtgggctcggagatgtgtataagagacagcctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagcctgtctcttatacacatctgacgctgccgacga

SEQ ID NO: 96 - DNA, 123 bp, synthetic construct
  source 1..123: mol_type = other DNA
caatgcatgggaatgaaggggtttttgtactctcaagatttcgaaaaatcttgctgagcctacaaagataaggcttcatgccgaattcaagcaccccatcattgatggggtgcttttcgtatt

SEQ ID NO: 97 - DNA, 123 bp, synthetic construct
  source 1..123: mol_type = other DNA
gcttgaggttgtccatgtttgtttttgtactctcaagatttcgaaaaatcttgctgagcctacaaagataaggcttcatgccgaattcaagcaccccatcattgatggggtgcttttcgtatt

SEQ ID NO: 98 - DNA, 123 bp, synthetic construct
  source 1..123: mol_type = other DNA
aagcaagaagagagccccaggtttttgtactctcaagatttcgaaaaatcttgctgagcctacaaagataaggcttcatgccgaattcaagcaccccatcattgatggggtgcttttcgtatt

SEQ ID NO: 99 - DNA, 123 bp, synthetic construct
  source 1..123: mol_type = other DNA
ttcaggcacatggatcgaatgtttttgtactctcaagatttcgaaaaatcttgctgagcctacaaagataaggcttcatgccgaattcaagcaccccatcattgatggggtgcttttcgtatt

SEQ ID NO: 100 - RNA, 123 nt, synthetic construct
  source 1..123: mol_type = other RNA
caatgcatgggaatgaaggggtttttgtactctcaagatttcgaaaaatcttgctgagcctacaaagataaggcttcatgccgaattcaagcaccccatcattgatggggtgcttttcgtatt

SEQ ID NO: 101 - RNA, 123 nt, synthetic construct
  source 1..123: mol_type = other RNA
gcttgaggttgtccatgtttgtttttgtactctcaagatttcgaaaaatcttgctgagcctacaaagataaggcttcatgccgaattcaagcaccccatcattgatggggtgcttttcgtatt

SEQ ID NO: 102 - RNA, 123 nt, synthetic construct
  source 1..123: mol_type = other RNA
aagcaagaagagagccccaggtttttgtactctcaagatttcgaaaaatcttgctgagcctacaaagataaggcttcatgccgaattcaagcaccccatcattgatggggtgcttttcgtatt

SEQ ID NO: 103 - RNA, 123 nt, synthetic construct
  source 1..123: mol_type = other RNA
ttcaggcacatggatcgaatgtttttgtactctcaagatttcgaaaaatcttgctgagcctacaaagataaggcttcatgccgaattcaagcaccccatcattgatggggtgcttttcgtatt

SEQ ID NO: 104 - DNA, 57 bp, synthetic construct
  source 1..57: mol_type = other DNA
tcgtcggcagcgtcagatgtgtataagagacaggagtctatgaaatgacaccatatc

SEQ ID NO: 105 - DNA, 50 bp, synthetic construct
  source 1..50: mol_type = other DNA
gtctcgtgggctcggagatgtgtataagagacagtcactagcctgtggag

SEQ ID NO: 106 - DNA, 53 bp, synthetic construct
  source 1..53: mol_type = other DNA
tcgtcggcagcgtcagatgtgtataagagacagcctgaccctgccgtgtacca

SEQ ID NO: 107 - DNA, 54 bp, synthetic construct
  source 1..54: mol_type = other DNA
gtctcgtgggctcggagatgtgtataagagacaggctctgggcagaacctggcc

SEQ ID NO: 108 - DNA, 25 bp, synthetic construct
  source 1..25: mol_type = other DNA
gcatttgtttgagaatcaaaatcgg

SEQ ID NO: 109 - DNA, 24 bp, synthetic construct
  source 1..24: mol_type = other DNA
gatttgtttgagaatcaaaatcgg

SEQ ID NO: 110 - DNA, 22 bp, synthetic construct
  source 1..22: mol_type = other DNA
gttgtttgagaatcaaaatcgg

SEQ ID NO: 111 - DNA, 21 bp, synthetic construct
  source 1..21: mol_type = other DNA
gtgtttgagaatcaaaatcgg

SEQ ID NO: 112 - DNA, 19 bp, synthetic construct
  source 1..19: mol_type = other DNA
gtttgagaatcaaaatcgg

SEQ ID NO: 113 - DNA, 17 bp, synthetic construct
  source 1..17: mol_type = other DNA
gtgagaatcaaaatcgg

SEQ ID NO: 114 - DNA, 25 bp, synthetic construct
  source 1..25: mol_type = other DNA
gttctcaatgcatgggaatgaaggg

SEQ ID NO: 115 - DNA, 24 bp, synthetic construct
  source 1..24: mol_type = other DNA
gtctcaatgcatgggaatgaaggg

SEQ ID NO: 116 - DNA, 23 bp, synthetic construct
  source 1..23: mol_type = other DNA
gctcaatgcatgggaatgaaggg

SEQ ID NO: 117 - DNA, 22 bp, synthetic construct
  source 1..22: mol_type = other DNA
gtcaatgcatgggaatgaaggg

SEQ ID NO: 118 - DNA, 21 bp, synthetic construct
  source 1..21: mol_type = other DNA
gcaatgcatgggaatgaaggg

SEQ ID NO: 119 - DNA, 20 bp, synthetic construct
  source 1..20: mol_type = other DNA
gaatgcatgggaatgaaggg

SEQ ID NO: 120 - DNA, 19 bp, synthetic construct
  source 1..19: mol_type = other DNA
gatgcatgggaatgaaggg

SEQ ID NO: 121 - DNA, 18 bp, synthetic construct
  source 1..18: mol_type = other DNA
gtgcatgggaatgaaggg

SEQ ID NO: 122 - DNA, 17 bp, synthetic construct
  source 1..17: mol_type = other DNA
ggcatgggaatgaaggg

SEQ ID NO: 123 - DNA, 20 bp, synthetic construct
  source 1..20: mol_type = other DNA
gactcctcgttgtttacccc

SEQ ID NO: 124 - DNA, 20 bp, synthetic construct
  source 1..20: mol_type = other DNA
attaccctagcaagttgatt

SEQ ID NO: 125 - DNA, 20 bp, synthetic construct
  source 1..20: mol_type = other DNA
tattaccctagcaagttgat

SEQ ID NO: 126 - DNA, 20 bp, synthetic construct
  source 1..20: mol_type = other DNA
cttcggcagtaaagaataaa

SEQ ID NO: 127 - DNA, 20 bp, synthetic construct
  source 1..20: mol_type = other DNA
gctagtgattgcagctgtgt

SEQ ID NO: 128 - DNA, 20 bp, synthetic construct
  source 1..20: mol_type = other DNA
acagggggccagaacttcgg

SEQ ID NO: 129 - DNA, 20 bp, synthetic construct
  source 1..20: mol_type = other DNA
caaaaaatctctgggtccag

SEQ ID NO: 130 - DNA, 20 bp, synthetic construct
  source 1..20: mol_type = other DNA
gacggcagccttctcctcag

SEQ ID NO: 131 - DNA, 20 bp, synthetic construct
  source 1..20: mol_type = other DNA
tagctctcttaaggagtgca

SEQ ID NO: 132 - DNA, 20 bp, synthetic construct
  source 1..20: mol_type = other DNA
catggatcgaattgaataca

SEQ ID NO: 133 - DNA, 20 bp, synthetic construct
  source 1..20: mol_type = other DNA
gactcctcgttgtttacccc

SEQ ID NO: 134 - DNA, 20 bp, synthetic construct
  source 1..20: mol_type = other DNA
attaccctagcaagttgatt

SEQ ID NO: 135 - DNA, 20 bp, synthetic construct
  source 1..20: mol_type = other DNA
tattaccctagcaagttgat

SEQ ID NO: 136 - DNA, 20 bp, synthetic construct
  source 1..20: mol_type = other DNA
cttcggcagtaaagaataaa

SEQ ID NO: 137 - DNA, 20 bp, synthetic construct
  source 1..20: mol_type = other DNA
gctagtgattgcagctgtgt

SEQ ID NO: 138 - DNA, 20 bp, synthetic construct
  source 1..20: mol_type = other DNA
tgcagatagatgaggagcca

SEQ ID NO: 139 - DNA, 20 bp, synthetic construct
  source 1..20: mol_type = other DNA
gcagatagatgaggagccaa

SEQ ID NO: 140 - DNA, 20 bp, synthetic construct
  source 1..20: mol_type = other DNA
cgacaggtttccaaagctgt

SEQ ID NO: 141 - DNA, 20 bp, synthetic construct
  source 1..20: mol_type = other DNA
tcagatacaaaattagagat

SEQ ID NO: 142 - DNA, 20 bp, synthetic construct
  source 1..20: mol_type = other DNA
cagatacaaaattagagatg

SEQ ID NO: 143 - DNA, 20 bp, synthetic construct
  source 1..20: mol_type = other DNA
gactcctcgttgtttacccc

SEQ ID NO: 144 - DNA, 20 bp, synthetic construct
  source 1..20: mol_type = other DNA
attaccctagcaagttgatt

SEQ ID NO: 145 - DNA, 20 bp, synthetic construct
  source 1..20: mol_type = other DNA
tattaccctagcaagttgat

SEQ ID NO: 146 - DNA, 20 bp, synthetic construct
  source 1..20: mol_type = other DNA
cttcggcagtaaagaataaa

SEQ ID NO: 147 - DNA, 20 bp, synthetic construct
  source 1..20: mol_type = other DNA
gctagtgattgcagctgtgt

SEQ ID NO: 148 - DNA, 20 bp, synthetic construct
  source 1..20: mol_type = other DNA
gactcctcgttgtttacccc

SEQ ID NO: 149 - DNA, 20 bp, synthetic construct
  source 1..20: mol_type = other DNA
attaccctagcaagttgatt

SEQ ID NO: 150 - DNA, 20 bp, synthetic construct
  source 1..20: mol_type = other DNA
tattaccctagcaagttgat

SEQ ID NO: 151 - DNA, 20 bp, synthetic construct
  source 1..20: mol_type = other DNA
cttcggcagtaaagaataaa

SEQ ID NO: 152 - DNA, 20 bp, synthetic construct
  source 1..20: mol_type = other DNA
gctagtgattgcagctgtgt

SEQ ID NO: 153 - DNA, 20 bp, synthetic construct
  source 1..20: mol_type = other DNA
caatgcatgggaatgaaggg

SEQ ID NO: 154 - DNA, 20 bp, synthetic construct
  source 1..20: mol_type = other DNA
ttcccaatcaacttgctagg

SEQ ID NO: 155 - DNA, 20 bp, synthetic construct
  source 1..20: mol_type = other DNA
gcttgaggttgtccatgttt

SEQ ID NO: 156 - DNA, 20 bp, synthetic construct
  source 1..20: mol_type = other DNA
aagcaagaagagagccccag

SEQ ID NO: 157 - DNA, 20 bp, synthetic construct
  source 1..20: mol_type = other DNA
ttcaggcacatggatcgaat

SEQ ID NO: 158 - RNA, 20 nt, synthetic construct
  source 1..20: mol_type = other RNA
gactcctcgttgtttacccc

SEQ ID NO: 159 - RNA, 20 nt, synthetic construct
  source 1..20: mol_type = other RNA
attaccctagcaagttgatt

SEQ ID NO: 160 - RNA, 20 nt, synthetic construct
  source 1..20: mol_type = other RNA
tattaccctagcaagttgat

SEQ ID NO: 161 - RNA, 20 nt, synthetic construct
  source 1..20: mol_type = other RNA
cttcggcagtaaagaataaa

SEQ ID NO: 162 - RNA, 20 nt, synthetic construct
  source 1..20: mol_type = other RNA
gctagtgattgcagctgtgt

SEQ ID NO: 163 - RNA, 20 nt, synthetic construct
  source 1..20: mol_type = other RNA
acagggggccagaacttcgg

SEQ ID NO: 164 - RNA, 20 nt, synthetic construct
  source 1..20: mol_type = other RNA
caaaaaatctctgggtccag

SEQ ID NO: 165 - RNA, 20 nt, synthetic construct
  source 1..20: mol_type = other RNA
gacggcagccttctcctcag

SEQ ID NO: 166 - RNA, 20 nt, synthetic construct
  source 1..20: mol_type = other RNA
tagctctcttaaggagtgca

SEQ ID NO: 167 - RNA, 20 nt, synthetic construct
  source 1..20: mol_type = other RNA
catggatcgaattgaataca

SEQ ID NO: 168 - RNA, 20 nt, synthetic construct
  source 1..20: mol_type = other RNA
gactcctcgttgtttacccc

SEQ ID NO: 169 - RNA, 20 nt, synthetic construct
  source 1..20: mol_type = other RNA
attaccctagcaagttgatt

SEQ ID NO: 170 - RNA, 20 nt, synthetic construct
  source 1..20: mol_type = other RNA
tattaccctagcaagttgat

SEQ ID NO: 171 - RNA, 20 nt, synthetic construct
  source 1..20: mol_type = other RNA
cttcggcagtaaagaataaa

SEQ ID NO: 172 - RNA, 20 nt, synthetic construct
  source 1..20: mol_type = other RNA
gctagtgattgcagctgtgt

SEQ ID NO: 173 - RNA, 20 nt, synthetic construct
  source 1..20: mol_type = other RNA
tgcagatagatgaggagcca

SEQ ID NO: 174 - RNA, 20 nt, synthetic construct
  source 1..20: mol_type = other RNA
gcagatagatgaggagccaa

SEQ ID NO: 175 - RNA, 20 nt, synthetic construct
  source 1..20: mol_type = other RNA
cgacaggtttccaaagctgt

SEQ ID NO: 176 - RNA, 20 nt, synthetic construct
  source 1..20: mol_type = other RNA
tcagatacaaaattagagat

SEQ ID NO: 177 - RNA, 20 nt, synthetic construct
  source 1..20: mol_type = other RNA
cagatacaaaattagagatg

SEQ ID NO: 178 - RNA, 20 nt, synthetic construct
  source 1..20: mol_type = other RNA
gactcctcgttgtttacccc

SEQ ID NO: 179 - RNA, 20 nt, synthetic construct
  source 1..20: mol_type = other RNA
attaccctagcaagttgatt

SEQ ID NO: 180 - RNA, 20 nt, synthetic construct
  source 1..20: mol_type = other RNA
tattaccctagcaagttgat

SEQ ID NO: 181 - RNA, 20 nt, synthetic construct
  source 1..20: mol_type = other RNA
cttcggcagtaaagaataaa

SEQ ID NO: 182 - RNA, 20 nt, synthetic construct
  source 1..20: mol_type = other RNA
gctagtgattgcagctgtgt

SEQ ID NO: 183 - RNA, 20 nt, synthetic construct
  source 1..20: mol_type = other RNA
gactcctcgttgtttacccc

SEQ ID NO: 184 - RNA, 20 nt, synthetic construct
  source 1..20: mol_type = other RNA
attaccctagcaagttgatt

SEQ ID NO: 185 - RNA, 20 nt, synthetic construct
  source 1..20: mol_type = other RNA
tattaccctagcaagttgat

SEQ ID NO: 186 - RNA, 20 nt, synthetic construct
  source 1..20: mol_type = other RNA
cttcggcagtaaagaataaa

SEQ ID NO: 187 - RNA, 20 nt, synthetic construct
  source 1..20: mol_type = other RNA
gctagtgattgcagctgtgt

SEQ ID NO: 188 - RNA, 20 nt, synthetic construct
  source 1..20: mol_type = other RNA
caatgcatgggaatgaaggg

SEQ ID NO: 189 - RNA, 20 nt, synthetic construct
  source 1..20: mol_type = other RNA
ttcccaatcaacttgctagg

SEQ ID NO: 190 - RNA, 20 nt, synthetic construct
  source 1..20: mol_type = other RNA
gcttgaggttgtccatgttt

SEQ ID NO: 191 - RNA, 20 nt, synthetic construct
  source 1..20: mol_type = other RNA
aagcaagaagagagccccag

SEQ ID NO: 192 - RNA, 20 nt, synthetic construct
  source 1..20: mol_type = other RNA
ttcaggcacatggatcgaat

SEQ ID NO: 193 - protein, 1377 aa, synthetic construct
MNKPYSIGLAIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDAIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMGKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGED

SEQ ID NO: 194 - DNA, 4131 bp, synthetic construct
  source 1..4131: mol_type = other DNA
atgaacaagccttattcaataggattagctatagggacaaattctgtggggtggagtataatcaccgacgattacaaggtgcctgcaaagaagatgcgcgtgctcggcaatacagacaaggaatatattaagaagaacctgatcggggccctcctttttgacggtggcaacacagcagctgaccgccgcctcaagaggaccgctcggagacggtatactcgccggcgtaatcggatcctgtatttgcaggaaatttttgctgaagaaatgtctaaggtggatgattcattctttcaccggctcgaagactcctttctggtggaggaagacaagaggggctcaaagtacccaatcttcgccacaatgcaagaagagaaatactaccacgagaagtttcccacaatctatcatctcaggaaagagctggccgataaaaaagagaaggccgatttgcgactggtttacttggccttggcacacatcataaagttccggggacactttctgattgaagacgaccgttttgacgtccgcaacactgatatacagaagcaataccaagcgttccttgagatctttgacaccacatttgaaaacaaccatctgctgagccaaaatgtggacgtggaagccattctgactgataagatctctaaatctgccaaaaaggacagaatccttgcccagtaccccaaccagaagtcaactggcattttcgccgagtttctgaagttgatagttggcaatcaggccgattttaagaagcacttcaatttggaggacaaaacgcctctccaattcgccaaggactcatatgatgaggacctggagaatctgcttggccaaatcggggatgagttcgctgatctttttagcgtggcaaagaagctctatgactctgtactcctgagcggaatcctgacagttaccgatctttcaacaaaggcacccctgagtgcaagcatgattcaacgctacgacgagcaccatgaggatctgaaacatctgaagcagttcgtcaaggcttctctgcctgaaaactatcgggaggtcttcgccgactcatctaaggacggctacgccggatacatcgagggaaagacaaatcaggaggctttctacaagtacctgttgaagctgcttacaaaacaggaggggagcgaatacttcctggagaagatcaaaaacgaggacttcctgcgtaaacagaggactttcgataatggctccattcctcaccaggtgcatctcacggaactgagagctatcattagacgtcagagtgagtattacccatttctgaaggagaaccaagaccgaatcgaaaaaattctgacgttccggatcccttactatgtcggacctttagctagggaaaaaagtgacttcgcctggatgacccgaaagacagatgatagtatcagaccatggaactttgaagacctggtggacaaagagaagagcgccgaggcttttattcacaggatgaccaataatgatctctatctgcctgaagagaaggtgctgcccaaacacagtctcatctacgaaaaatttacagtctataacgaactgacaaaggtccgctttctggctgaaggattcaaggactttcaatttctgaaccggaagcagaaggaaactatctttaactcattgtttaaggaaaagaggaaggttaccgaaaaagacatcatctcctttttaaacaaggtagatgggtacgaagggattgccattaaaggcattgagaaacagtttaacgccagcctttcaacctaccatgatctcaagaagatcctcggaaaagatttccttgacaataccgacaacgaacttatcctggaggatatagtgcagacactcactctgttcgaggacagggaaatgataaagaagtgcctcgacatatataaagacttctttaccgagagtcaactgaaaaagttgtatagaaggcattacaccggttggggccgactgagtgcaaaactcattaacggcatccggaataaggagaatcaaaagactatcctcgattacctcatcgatgacggaagcgcaaacagaaacttcatgcaactcatcaacgatgatgacctgtctttcaaaccaattatagacaaagccaggactgggagccatagtgacaatctgaaggaagtggtgggagagctggcaggcagccccgcaattaagaaggggatcctgcagagcctcaaaattgtcgatgaactcgtgaaggtcatgggctatgaacctgaacagattgttgtagagatggcccgagagaaccagactactgcgaagggacttagccggagcagacaacgactgaccactttgcgagagagtctggcgaacctgaagtctaatattctcgaggaaaaaaagccaaagtacgtgaaggaccaggtggagaatcaccacctgagcgacgacagactctttctgtattatctgcagaacggcagagatatgtatacgaagaaggcactggacatagacaacctgagtcagtatgacatcgatgccattatccctcaggccttcatcaaagacgattcaatcgacaatcgcgtacttgttagcagtgcgaaaaaccggggaaagtctgatgacgtcccatccatcgaaatagtgaaggcaaggaagatgttctggaagaatctgctggatgccaaattaatgtcacaacggaagtacgacaacctgacaaaggcagaaagggggggcttaacaagcgacgataaggcaaggtttatccagaggcagttggtcgagaccaggcaaatcaccaaacacgtcgcccggatcctggatgaacgcttcaacaatgaagtcgacaatggcaaaaaaatctgtaaagtcaagatagtgacactgaagtcaaatctggtgagcaacttccggaaagaattcggcttctataaaattcgcgaagtgaacgactatcaccatgcgcacgacgcttacctgaatgcagtcgtggcgaaagccattttgaccaagtacccccagctggagcctgagtttgtgtacggaatgtaccgacaaaagaagctgagcaagattgtacacgaggataaggaagagaaatactccgaggccactcggaagatgttcttctactctaatctgatgaacatgtttaagagagtggtgaggttggcagacggctccattgttgtaaggccagtgatcgagactgggcgatacatgggcaagacagcgtgggacaagaagaagcatttcgcaaccgtacggaaagtcctgtcctacccgcagaataacattgtgaagaagacagaaatacaaaccggtggtttctcaaaagagtccattttagcccatggcaacagtgacaaattgattccacggaagaccaaagatatttatctggaccctaaaaaatacggcggattcgactcaccgatcgtggcatacagcgtattggtggtggccgatattaagaagggtaaagcccagaaactcaagactgttaccgagctcctgggtatcactataatggagagaagccggtttgagaagaaccctagcgcctttttggaatccaaggggtatctgaacattcgggacgataagctgatgatcttgcctaaatacagcctttttgaactggagaatggacgaaggcgcctgcttgcctcagcgggggaactgcagaaaggcaatgagctggcccttcctacccagttcatgaaatttttgtatctggctagtaggtataacgagtcaaaaggcaagccagaggagatcgaaaagaagcaggaatttgtaaaccagcatgtgtcatactttgatgatatcctgcagttaatcaatgacttcagtaaacgagtcattctcgcagacgccaacttggagaaaattaataagctgtaccaggacaacaaagagaatataccagtcgacgagcttgcaaataacattattaacctgttcacttttacatccctgggggcccctgctgcgttcaaatttttcgacaaaatcgtggatcgaaagcgatatacatccactaaggaagttctgaacagcactctcatccaccagtctatcactggcctttacgaaacgcgtattgacttggggaaactcggagaggac

SEQ ID NO: 195 - RNA, 102 nt, synthetic construct
  source 1..102: mol_type = other RNA
gttttagagctgtgcgaacacagcacgttaaaataaggcagtgatttttaatccagtccgtattcagcttgaaaaagtgagcaccgattcggtgcttttttt

SEQ ID NO: 196 - DNA, 102 bp, synthetic construct
  source 1..102: mol_type = other DNA
gttttagagctgtgcgaacacagcacgttaaaataaggcagtgatttttaatccagtccgtattcagcttgaaaaagtgagcaccgattcggtgcttttttt

SEQ ID NO: 197 - protein, 1130 aa, synthetic construct
MTNGKILGLAIGIASVGVGIIEAKTGKVVHANSRLFSAANAENNAERRGFRGSRRLNRRKKHRVKRVRDLFEKYEIVTDFRNLNLNPYELRVKGLTEQLTNEELFAALRTISKRRGISYLDDAEDDSTGSTDYAKSIDENRRLLKTKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCSFYPDEYRASKASYTAQEYNFLNDLNNLKVPTETGKLSTEQKEALVEFAKSTATLGPAKLLKEIAKILDCKVDEIKGYREDDKGKPDLHTFEPYRKLKFNLDSVNIDDLSREVLDKLADILTLNTEREGIEDAIRHNLPNQFTEGQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNGTDKLPDEVFHGNKQLETKIRLWYQQGERCLYSGKPIPIQELVHNSNNFEIDAILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKISVIRGQFTSQLRRKWKIDKSRETYHHHAVDALIIAASSQLKLWEKQDNPMFVDYGNNQVVDKQTGEILSVSDDEYKELVFQPPYQGFVNMISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLVCKYSKKGKGTPIKSLKYYDKKLGNCIDITPEGSKNEVVLQSLNPWRADVYFNPETLKYELLGLKYSDLSFEKGTGKYHISQEKYDVIKEKEGIGKKSEFKFTLYRNDLILIKDTASGEQEIYRFLSRTMPNVKHYAELKPYDKEKFDNVQELVEALGEADKVGRCIKGLNKSNLSIYKVRTDVLGNKYFVKKEGDKPKLDFKNNKK

SEQ ID NO: 198 - DNA, 3390 bp, synthetic construct
  source 1..3390: mol_type = other DNA
atgacaaacggcaaaattctgggtctggccatcggaatcgctagcgttggcgtgggaatcattgaagcgaagacaggtaaagtcgtccatgcaaattctcgattgttctccgcagctaacgctgaaaacaatgcggagagaaggggtttcagaggctctaggcggctcaaccggcgcaagaagcacagggtaaaaagagtgcgagatctctttgagaaatatgagatcgtgactgattttagaaacctgaatctgaacccatatgagctgagagtgaaaggacttacggaacagctcactaatgaagagttgttcgccgccctgcggaccatcagcaaacgccgaggaatttcctaccttgatgacgccgaagatgacagtaccggtagcacagattatgccaagagcattgatgagaacaggagactgctgaagactaagacacctggacagatacaattggaacggctcgagaagtacggccagctgaggggtaacttcaccgtttatgacgaaaatggggaggcccatagactgataaatgtgttctcaacttctgactatgaaaaggaggcccggaaaatcctcgagactcaagccgactacaacaagaagattacagccgagtttattgacgattacgtggaaattttaacccagaaaaggaagtattaccacgggccaggaaatgaaaagagccgcaccgactatgggagattcagaacggatggaacaaccttagagaatatctttggaatccttattggtaaatgctctttctatcctgacgagtatcgcgccagcaaagcctcctataccgctcaggagtacaacttcttgaatgatttgaacaatttgaaggttccgacggagactggcaagctgagtaccgagcaaaaggaggcccttgtggaattcgccaagtctactgcaacattaggtcctgctaaacttctgaaggagattgccaaaattttggactgcaaagtcgatgaaatcaaggggtaccgtgaggatgataaagggaaaccagacctgcacacctttgagccctatagaaagttgaaattcaatctggacagcgtcaacattgacgatttgagtcgcgaagtgctggacaagctggcagacattttgacacttaacactgaaagggagggcattgaggatgccatcaggcataacctgcccaaccaatttactgagggccagatctccgaaatcatcaaggtgcgcaaaagccagagcactgctttcaacaaggggtggcacagcttctctgccaagctcatgaacgaattgattcccgagctctatgccacaagcgacgaacagatgactatacttactcggctggagaaatttaaggtcaataaaaaatcctccaaaaacaccaagacgattgacgagaaagaggtcactgatgaaatctacaatccagttgtagccaagtctgtccggcaaacgatcaagatcattaacgctgctgtgaagaaatatggagactttgataagattgtgattgaaatgcctcgcgacaagaatgcggacgatgagaagaagtttatcgataagagaaacaaagaaaataagaaagaaaaggatgatgccctgaagcgggcagcttacctttataatggaaccgataagctgccagatgaggtgtttcacggaaacaagcaacttgaaaccaagattcgcctgtggtaccagcagggagaacggtgtttgtactcaggcaagcctatcccaatccaggagttggtccacaactccaataacttcgaaatcgatgcgattctgcccctgtccctgagttttgacgactccctggccaacaaggtgcttgtgtatgcttggaccaaccaagagaagggccagaagacgccctaccaggtgattgattctatggatgcggcgtggtcctttcgcgagatgaaggactatgtgctcaagcaaaaaggcctcggcaaaaagaaacgggattatcttttgaccaccgagaacattgacaagattgaagtgaagaaaaaattcatcgagcgcaacttggtcgataccagatatgcctctagggttgtgctgaactcactgcagtctgctttgagagagctgggtaaagacactaaaattagtgtaatcaggggccagttcacaagtcagcttaggcggaaatggaagatcgacaagtcacgcgagacatatcatcatcacgcagtcgacgcactgataattgcagcttcaagtcagctcaagttgtgggagaaacaggataaccctatgtttgtcgactatggaaacaatcaggtcgtcgataagcagaccggggaaattttaagtgtgtccgatgacgagtataaggagcttgtctttcagccaccgtaccagggctttgtcaacatgattagtagcaagggttttgaggacgaaattttgttcagctaccaggtcgattccaaatacaatagaaaagtatccgacgcaaccatatattctactcgcaaggccaagattggcaaagataagaaggaagagacctatgtattggggaagatcaaagacatttactcacaaaatggattcgacaccttcattaagaagtacaacaaagataagacacagtttttgatgtaccagaaagatccactgacatgggaaaacgtgatcgaagttatactgcgtgactaccccacgactaaaaagagtgaggacggaaaaaacgacgtgaagtgcaacccgtttgaagaataccggagagaaaacggtctggtgtgtaagtactctaagaaaggaaaggggacccctattaaatccctcaaatactacgacaaaaaactcgggaactgcatcgatatcaccccggaaggttccaaaaatgaagtcgtgcttcaatccttgaatccgtggagggcagatgtgtactttaacccagaaaccttgaagtatgaattactgggacttaaatacagtgatctctcatttgaaaagggcactggaaaataccatatctctcaggagaagtacgacgtcattaaggaaaaagaagggatcgggaaaaaatccgagttcaagttcacattgtataggaacgacctgatccttattaaagacacagccagcggtgagcaggagatttaccgatttctgtctagaaccatgcctaacgtcaagcactatgcggagctgaagccctatgacaaagaaaaatttgataacgtccaggaactcgtcgaggcgctgggcgaagccgacaaggtaggccgctgtataaaggggctgaacaaaagcaacctcagcatctataaagttaggacagatgtgctcgggaacaaatacttcgttaagaaggaaggggacaagcccaagctggattttaagaacaataaaaag

SEQ ID NO: 199 - RNA, 100 nt, synthetic construct
  source 1..100: mol_type = other RNA
gtttttgtactctcaagatttcgaaaaatcttgctgagcctacaaagataaggctttatgccgaattcaagcaccccatgttttgacatggggtgctttt

SEQ ID NO: 200 - DNA, 100 bp, synthetic construct
  source 1..100: mol_type = other DNA
gtttttgtactctcaagatttcgaaaaatcttgctgagcctacaaagataaggctttatgccgaattcaagcaccccatgttttgacatggggtgctttt

SEQ ID NO: 201 - protein, 1368 aa, synthetic construct
MRKPYSIGLAIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADRDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLNGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKDRKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKHEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDAIIPQSFIKDNSIDNIVLTTQASNRGKSDNVPNIEIVNKMKSFWYKQLKNGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESILPKGNSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDEIMQIIVEYSNRYILADSNLIKIQNLYKEKDNFSIEEQAINMLNLFTFTDLGAPAAFKFFNGDIDRKRYSSTNEIINSTLIYQSPTGLYETRIDLSKLGGK

SEQ ID NO: 202 - DNA, 4104 bp, synthetic construct
  source 1..4104: mol_type = other DNA
atgcgcaaaccttactcaattggcctggcaatcgggactaattctgttggctgggctgtgattactgatgattacaaggtgccaagtaagaaaatgaggattcagggcacgactgatcggaccagcattaagaagaatctcattggggccctcctgttcgataatggcgagactgccgaggccactcgattaaagagaacaacaaggaggaggtacaccagacggaagtaccgaataaaggaactgcaaaagatcttcagcagcgaaatgaatgagctcgacattgcttttttccctagactgtctgagagttttcttgtgagtgacgacaaagaattcgagaatcatccgatttttggaaaccttaaagatgagataacttatcataacgattaccctactatttatcacttgcgacagacacttgcagaccgtgaccagaaggccgatcttaggctcatttatctcgctctggcccacattattaaatttcgggggcactttttgatcgaaggcaatctggacagtgagaacacggacgtacacgtgctgtttctgaacctggtgaacatatataataacctgttcgaggaagatatagttgaaaccgcatccatagacgctgagaagattcttacctcaaaaacttccaaatccaggcggctcgagaatcttatagctgagattcctaaccagaagcggaacatgttgtttggcaacctcgtgtctctggctctcggcctgacaccaaattttaaaaccaattttgagctgctggaggatgcaaagttacagatctccaaggattcatatgaagaagacctcgacaacttgttggcacagattggggatcagtacgcagatctctttatcgccgctaaaaagctttctgacgcaatattactgtctgacatcatcaccgtgaagggcgcctccactaaagcgcctctttcagcatccatggtgcagagatatgaagagcatcaacaggacctcgctctcctgaagaatctcgtgaaaaaacagattcctgagaagtataaggaaatcttcgataacaaggagaagaatggctatgcaggttatatcgatggcaagacctcccaggaggaattttacaagtacatcaagcccatacttcttaagctcaacggcacagagaagttgatcagcaaacttgagcgggaggacttcctgagaaagcaacgaacattcgacaacggatctattcctcaccagattcacctgaatgagctcaaggcaatcatccggaggcaggagaagttttatccctttctgaaggaaaatcagaagaaaatcgaaaagcttttcacatttaaaattccctattacgtcgggccactcgccaatggccagagtagcttcgcctggctgaagagacagtccaacgagtctatcaccccctggaacttcgaggaagtggtggatcaagaggcctcagcgcgcgccttcatagagaggatgactaacttcgatacctatttacccgaggagaaggttctgccaaagcacagcccactctacgaaatgtttatggtctataatgagctcaccaaggttaagtatcagaccgaggggatgaagaggcccgtctttctctcttccgaagacaaagaagaaatagtgaatctcctgttcaaaaaagaccggaaggtcactgtcaagcagctgaaggaggaatatttctccaaaatgaaatgcttccacaccgtgacaatcttgggcgtggaggatcggtttaatgcttctctgggcacgtaccatgacctgctcaaaatttttaaagataaagccttcttagacgatgaggccaatcaagatatcttggaagagatcgtatggactttaacgctttttgaggatcaagccatgattgaaagaaggctggtgaagtacgcggacgtgttcgaaaaatccgtccttaaaaagttaaagaaacgccattacacgggctggggacgtctttcccagaagcttattaatgggatcaaagacaaacaaactgggaagacaattctcggctttctgaaagacgacggtgtagccaaccgaaattttatgcagttaattaacgacagctccctggacttcgcaaagattatcaagcatgaacaggaaaaaaccatcaagaacgagtcattggaggaaacgattgcgaacctggcaggcagccccgccattaagaaaggcattcttcagtctattaaaattgtcgatgaaatcgttaagattatgggacagaacccagacaatattgttattgagatggcacgcgagaaccaatccacgatgcaaggaatcaaaaactcccgacagcgtctgcgcaagctcgaggaggtgcataagaacaccgggtccaagattttgaaagaatacaacgtgagtaatacgcagcttcagagcgataggctctatttatacctgctgcaggacggaaaggatatgtacaccggcaaggagttggactacgacaatcttagtcaatatgatattgatgcgatcatccctcagtctttcataaaagataactctatcgacaacatagtgctgactacacaagctagtaataggggcaagtcagacaacgtgcccaacatagagattgtgaacaaaatgaagtctttttggtataaacagctcaaaaatggggcaattagccagcgcaaattcgaccatttaaccaaggccgagcgtggcgcactgagcgatttcgataaggcaggctttatcaagcgccagctcgtcgagacacggcagataaccaaacatgtggctcaaatcctggacagtcggttcaattccaatcttacggaggactctaaatctaacagaaacgttaagataataactctcaagtcaaaaatggtgagtgacttccgaaaggactttggcttttacaagctgagagaagtaaatgattatcaccacgcccaggacgcatatctcaatgccgtcgtcggtactgccttacttaagaagtaccctaaactggaagcagagttcgtgtatggggattacaagcactacgatctcgctaagttaatgattcaaccggacagtagccttggaaaagccacaaccagaatgttcttctattctaacctcatgaatttcttcaaaaaagaaatcaaactggccgatgatactatatttacgaggccccagattgaagtgaacaccgaaactggggagattgtctgggataaggtaaaggacatgcagaccatcaggaaagtgatgtcctatccacaagtcaacatagtgatgaaaaccgaagtccagactggggggttttctaaggagagtatcctgcctaagggaaactcagacaaactgatcgcccgcaagaaatcctgggaccctaagaaatacggtggtttcgatagccctatcattgcatattcagtcctggtcgtcgctaagatagccaaaggcaaaacccagaaactcaagactattaaagagttggtcggtatcaaaatcatggagcaggacgaattcgaaaaggatccaattgcgtttctcgaaaagaagggctatcaggacatacagacctcttccatcatcaagctgccgaagtactctctctttgagcttgagaatggacgcaagagactgctggctagcgccaaagaactgcagaagggcaacgaactggccctccctaacaaatacgtaaagttcttgtatttagcatctcattacacaaaattcacaggtaaggaggaagatcgagaaaaaaagcgctcctatgtagagtcacacctgtattactttgacgagattatgcagattatcgttgagtattctaaccggtacattctcgccgacagcaatctgattaaaattcagaacttgtacaaagagaaggataactttagtatcgaggagcaagccattaatatgctcaatctcttcacttttacagatctcggcgcgccagccgctttcaagttctttaacggagatatagatcggaagcggtacagctctaccaacgagatcattaattctactctgatttaccagagtcccacagggttatacgagaccaggatcgacctcagtaagctggggggcaaa

SEQ ID NO: 203 - RNA, 102 nt, synthetic construct
  source 1..102: mol_type = other RNA
gttttagagctgtgttattcgaaaacaacacagcaagttaaaataaggcttgtccgtaatcaacttgaaaaagtgaacaccgattcggtgtttttttatttt

SEQ ID NO: 204 - DNA, 102 bp, synthetic construct
  source 1..102: mol_type = other DNA
gttttagagctgtgttattcgaaaacaacacagcaagttaaaataaggcttgtccgtaatcaacttgaaaaagtgaacaccgattcggtgtttttttatttt

SEQ ID NO: 205 - protein, 1129 aa, synthetic construct
MSNGKILGLAIGVASVGVGIIDAKTGNVIHANSRLFSAANAENNAERRGFRGARRLTRRKKHRVKRVRDLFEKYDISTDFRNLNLNPYELRVKGLTEQLTNEELFAALRTIAKRRGISYLDDAEDDSTGSSDYAKSIDENRRLLKTKTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKKSKALGASKLLKEIAKIVDCSVDDIKGYRVDNKDKPDLHTFEPYRKLKFNLSSIDIDELSRETLDKLADILTLNTEREGIEDTIKRNLPSQFTEEQISEIVQIRKNQSSAFNKGWHSFSAKLMNELIPELYVTSEEQMTILTRLEKFKVNKKSSKNTKTIDEKEITDEIYNPVVAKSVRQTIKIINAAVKKYGDFDKIVIEMPRDKNAEDEKKFIDKKEKENKKEKDDSLKRAAFLYNGTDNLPDGVFHGNKELKTKIRLWYQQGERCLYSGKLISIHDLVHNSNKFEIDAILPLSLSFDDSLANKVLVYAWTNQEKGQKTPYQVIDSMDAAWSFREMKDYVLKQKRLGKKKREYLLTTENIDKIEVKKKFIERNLVDTRYASRVVLNSLQTALKELGKDTKVSVVRGQFTSQLRRKWNIDKSRETYHHHAVDALIIAASSQLKLWQKQENPMFESYGENQVVNKETGEILSISDDKYKELVFQPPYQGFVNTISSKGFEDEILFSYQVDSKFNRKVSDATIYSTRKAKLGKDKKDETYVLGKIKDIYSQDGFDTFIKRYKKDKTQFLMYQKDPLTWENVIEVILRDYPSEKLSEDGKKTVKCNPFEEYRRENGLICKYSKKGNGTPIKSLKYYDKKLGNCIDITPEKSKNRVVLRQISPWRADIYFNLETLKYELMGLKYSDLSFEKGTGKYHISQEKYDAIREKEGIGKKSEFKFTLYRNDLILIKDTLNNCERMLRFGSKNDTSKHYVELKPLEKGTFDSEEEILPVLGKVAKSGQFIKGLNKPNISIYKVRTDVLGNKFFIKKEGDKPKLDFKNNNK

SEQ ID NO: 206 - DNA, 3387 bp, synthetic construct
  source 1..3387: mol_type = other DNA
atgtcaaatggcaaaatcttaggcttggccatcggggtggccagcgtcggggttggcataattgatgccaaaaccggcaacgtgatccacgcaaatagcaggctgtttagcgccgccaacgccgagaacaatgctgagcggaggggattccgcggcgcacgtaggctcacgaggcgcaaaaaacatagagtgaagcgggtccgtgacctgtttgaaaagtatgatatctcaacagatttccgcaacttaaatctgaacccctacgagctcagggtgaaaggcctgacagaacagcttaccaatgaagaactcttcgcagctttaagaactattgccaaacggcgcggcatctcctacttggatgacgcggaagacgattctaccggaagcagcgactacgcgaagtcaatcgacgaaaatagacgtcttctgaaaaccaaaactccagggcaaatccagctggagagactggagaagtacggacagctgaggggcaattttaccgtgtatgacgaaaacggagaagctcacagactgatcaatgttttttccacttccgattataaaaacgaagcccggaagatcctggagacgcagagcaactacaacaagcaaatcaccgatgagttcatcgaagattacattgagatattaactcaaaagcgtaaatactaccatggcccaggcaacgagaagagcaggaccgattacggcaggttccgaacagatggaactaccctggagaacatttttggcattcttattggaaaatgctcattctatccagaggaatatcgtgctagtaaggcaagctacaccgcccaagaattcaactttctgaatgacctgaataatctgaaggtccccaccgaaacgggcaagttatcaactgagcagaaggagtatttagtggattttgccaagaagtctaaggctctgggagcgtctaagcttctgaaggagattgccaagatagttgattgcagcgttgacgacatcaaggggtacagggtggataataaagacaagccagatctgcacacctttgagccatatagaaagttgaagtttaacttgagtagtatcgacatcgatgaactgtctagagagacactcgacaaactcgctgacattcttactctgaacacagaacgggaaggcatcgaggatacaatcaaaagaaaccttccctcacagtttaccgaggaacagataagcgagattgtccaaattcggaagaatcaatccagcgcctttaacaagggttggcactccttctcagcaaagttgatgaacgagttaatcccagagctgtacgtgacttcagaggagcagatgacaattctgaccaggttggaaaaatttaaggtgaacaagaagagctccaaaaacacaaagaccatcgatgaaaaggagattactgacgagatctataacccagtcgtcgcgaaatccgtgaggcaaactatcaagattatcaacgccgcggtgaaaaagtatggagactttgacaaaatcgtgattgagatgccacgtgacaagaatgcagaggatgagaaaaaatttattgacaaaaaggagaaggaaaataagaaggaaaaagatgatagcctgaagcgcgcagctttcctgtataacggcacagacaatttgccagacggagtatttcacggaaacaaggagctcaagactaaaattcgcttatggtatcaacaaggcgagaggtgcttgtatagcggcaaactgatatccatacacgacctcgtacacaacagtaacaagtttgagattgacgccatccttccacttagcctgagtttcgacgacagcctggcaaataaggtcttggtatatgcttggaccaatcaggagaaggggcaaaaaaccccgtaccaggtgatagatagcatggacgcggcatggagttttcgggaaatgaaggactacgttctcaaacagaagagactcggcaaaaaaaagcgtgaatacctgctgactaccgagaacattgacaaaatcgaagtcaaaaaaaagttcatcgagcgcaaccttgtggatacccgctatgcctcacgcgtcgtcctgaactctctgcagacagctctgaaagaactgggcaaggacaccaaagtgtctgtcgttaggggtcaatttacctcccagttgcgacgcaagtggaatatcgataagtccagagaaacataccatcatcacgcagtagacgcccttatcattgccgcatcttctcagcttaaactgtggcaaaagcaggaaaatcctatgtttgagtcttatggcgaaaatcaggtcgtcaataaggagacaggagagatcttatcaatatccgatgacaagtataaagaactggtgtttcaaccaccataccaagggtttgtcaacactatcagcagtaaaggcttcgaggatgagatcttgttttcatatcaggtggacagcaaattcaaccggaaagtttctgatgccaccatttatagtactcgcaaagcgaaacttggaaaggacaagaaggatgagacctacgtattggggaaaatcaaggacatttactctcaggacggctttgacaccttcattaagcgttacaaaaaggacaagacgcagttcctgatgtaccaaaaagatccactgacttgggaaaatgttattgaggtgatcctccgggattatccaagtgaaaaattgtcagaggacggcaaaaaaacagtgaagtgcaatccgtttgaagaatataggcgagagaatggtctgatctgtaaatactctaaaaagggcaacggaacccccatcaagtccctgaaatattacgacaagaaacttggtaactgcattgacatcacccctgagaaaagcaagaaccgcgtggtgctgaggcagatatcaccttggcgcgctgatatctacttcaacctggagaccttgaaatatgagctcatgggcttgaaatacagtgacctgtcttttgaaaaagggaccgggaagtatcacattagccaggaaaagtacgatgcgattagagaaaaagaaggcattggcaaaaagagcgagtttaagtttactttgtatcgaaacgatctcatcctgataaaagataccctgaacaattgtgagaggatgcttaggttcggatccaagaacgatacatctaagcactacgtggaactcaaacctttagagaagggcacctttgattccgaggaggagatccttccagtgctgggcaaggttgcgaaatccgggcagtttattaagggtcttaacaaacccaatatctcaatctataaggtgaggaccgatgtgcttggcaacaaattctttatcaagaaggaaggcgacaaacccaagctggatttcaagaataataacaag

SEQ ID NO: 207 - RNA, 98 nt, synthetic construct
  source 1..98: mol_type = other RNA
gtttttgtactctcaagcgaacttgcagagcctacaaagataaggcttcatgccgaattcaagcaccccatgtttacatggggtgcttttcgttgtgt

SEQ ID NO: 208 - DNA, 98 bp, synthetic construct
  source 1..98: mol_type = other DNA
gtttttgtactctcaagcgaacttgcagagcctacaaagataaggcttcatgccgaattcaagcaccccatgtttacatggggtgcttttcgttgtgt

SEQ ID NO: 209 - protein, 1345 aa, synthetic construct
MKKPYSIGLAIGTNSVGWAVVTDDYKVPAKKMKVLGNTDKSHIKKNLLGALLFDSGNTAADRRLKRTARRRYTRRRNRILYLQEIFAEEMSKVDDSFFHRLEDSFLVTEDKRGERHPIFGNLEEEVKYYENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEDLEELLGKIGDDYADLFTLAKNLYDAILLSGILTADDSSTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKILTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDAIIPQAFIKDNSIDNRVLTSSKENRGKSDDVPSKDVVRKMKPYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHEENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLSKLGGD

SEQ ID NO: 210 - DNA, 4035 bp, synthetic construct
  source 1..4035: mol_type = other DNA
atgaagaagccttactcaattggcctggctattggcactaattcagtgggatgggccgtcgttaccgatgattacaaggtacccgcaaagaagatgaaggtccttggtaatacagataaaagtcacataaagaagaatcttctcggagctcttctgttcgacagcgggaacacagctgccgataggcgactcaaaagaactgctcgcaggcgctatacaaggcgcagaaaccgcattctgtacctgcaggagatcttcgccgaagagatgtccaaagtggatgacagttttttccataggctcgaggatagcttcctggtgaccgaggacaaaaggggggagagacatcccattttcggtaatcttgaagaggaggttaagtactacgagaacttcccgactatatatcatctgcggcagtatctcgcagacaaccccgaaaaagtggacctgcgacttgtgtatcttgccctggcacatattataaaattcagaggccactttctgattgaggggaagtttgatacccggaataatgatgtgcagcgcctgtttcaggaattcttggctgtctacgacaatacatttgagaatagtagtttgcaggagcagaacgtgcaagtggaagagatcctgacagacaagatctccaagagcgccaaaaaagatagggtgctcaaattgttccctaatgagaaatccaacggcaggtttgccgaattcttgaaactgattgtgggaaaccaggctgactttaagaaacatttcgagcttgaagaaaaggcccctttgcagttttccaaagacacctacgaggaggatctggaggaactgctgggaaagatcggggatgactatgccgatctgtttaccctcgccaagaacctgtacgatgcgattctcttgtccggtatcctgacggcagacgacagttcaactaaagctccgctctctgccagcatgattcagcgatacaatgagcatcagatggatctggcccagctcaagcagttcatccgacagaaactcagcgataagtacaacgaggtgtttagcgacgtgtccaaagacgggtacgcaggctacattgacggcaagaccaaccaagaagcgttctacaaatacctgaaagggctgctcaacaagatagaaggatcaggttactttctggataaaatcgaacgggaggattttttgcgcaagcagcgaactttcgacaatgggtccatccctcatcagattcacctgcaggaaatgagagctattattaggagacaggctgaattttacccttttctggcagataaccaggatcggatcgagaaaatcttaacctttcggatcccatactatgtgggcccactggcccgtggcaaatccgacttcgcatggctgtcacggaagtccgccgataaaattacgccgtggaactttgatgaaattgtcgataaggaatcttccgctgaggcttttatcaatcgcatgaccaattacgatctgtacctgcctaatcagaaggtgttacccaagcatagcctgttgtatgaaaaattcactgtctacaatgaactcaccaaagtcaagtacaagacagaacagggcaaaaccgcctttttcgacgctaatatgaaacaggaaatttttgacggggtgttcaaagtctatagaaaggtcactaaggacaaactgatggattttctggagaaggaatttgatgagtttcgcatagttgatcttactggtttggataaagaaaataaggtcttcaatgcaagctacggtacataccacgacctttgtaaaattctcgacaaggatttcctcgacaactccaaaaatgaaaagattcttgaggatatcgtgttaaccctgaccctgtttgaagacagggaaatgatccggaagcggctggagaattactccgacctgttgactaaagagcaggtgaaaaagctcgagaggcgccattacaccggatgggggagactcagtgccgaacttatccatggaattcgaaacaaggagagcaggaagaccattctcgattatctgattgacgatggtaatagcaacagaaattttatgcagctgatcaacgatgatgcactgtcatttaaggaggaaattgcaaaagcccaggttatcggcgagaccgacaacctgaatcaggttgtgagtgacatcgcagggagccccgctatcaagaagggaatcctccagtccctcaagattgtcgacgagctcgtcaagatcatggggcatcagccagagaacattgtcgtggagatggcccgcgaaaaccaatttaccaaccaagggaggcggaacagccagcaaagactgaagggcttaacagatagcattaaagagttcggatctcagatacttaaagaacaccccgtcgaaaactcccagttgcagaatgaccgcctctttctgtattatctgcaaaacggaagggacatgtatacgggagaggagctggatatagattaccttagtcaatatgatatcgatgctataatcccccaagcctttatcaaggacaactctatagacaatagggtcctgacctctagcaaagagaatagaggcaagtccgatgacgtaccttctaaggatgtcgtgcgcaagatgaagccatactggagcaagctgctgtctgcaaagcttataacccaacgaaagttcgataatctgactaaggccgagcgcggcgggctgacagatgacgataaggccgggttcattaagcgccagctggtggaaacaagacaaatcactaaacacgtcgctcgaattcttgatgagcggtttaacacagaaacggacgaaaacaacaaaaagatccgccaggtaaaaattgtaaccctgaagagcaaccttgtttctaatttcagaaaggaattcgaactttacaaagtgcgtgaaatcaacgactaccatcatgcccatgacgcttatctgaacgctgtcatcgggaaggccctccttggggtctatcctcagctggagcctgaatttgtgtacggagattacccacactttcacgggcacgaagagaacaaggcaactgctaagaagttcttttattcaaatatcatgaatttttttaagaaagacgacgtcagaactgataaaaacggtgagatcatttggaagaaggacgaacatatcagtaatattaaaaaggtgcttagctatcctcaggtgaacatagttaaaaaagtagaggagcagacaggcgggttctccaaggaatccatactgccaaaaggcaacagcgataaactgatacctcggaagactaaaaaattctactgggataccaagaagtacgggggatttgacagccccattgtcgcctactctatattggttattgcggacatcgaaaagggcaaatccaaaaagcttaaaactgtcaaagccctggtgggggttaccatcatggagaaaatgacctttgaacgcgatcccgtagcattcctcgaacgcaagggctaccgcaacgttcaggaggagaatatcatcaagttgcccaaatattctctctttaagctggagaacggcagaaagcgcctgctcgcatccgcaagggagttacagaaaggcaacgaaattgtactgcccaatcacctcggaaccctgctgtatcacgccaaaaatatccataaagtcgacgaacctaagcacttagactatgtcgataaacacaaggacgaatttaaagagctgctggacgtggttagcaatttctcaaagaaatacacgctggcggaaggtaatctggagaaaattaaagagttgtacgctcagaataacggggaggatcttaaagaactggcgtcctcatttatcaacctgctgaccttcaccgccatcggcgcgcctgctacatttaaattcttcgataagaatatcgatagaaagagatatacttccacgaccgaaattttgaacgctaccctgattcaccaatctattaccgggttatatgagacacgaattgatctgagcaaactggggggggat

SEQ ID NO: 211 - RNA, 98 nt, synthetic construct
  source 1..98: mol_type = other RNA
gttttagagctgtgtcgaaacacagcaagttaaaataaggtttatccgtattcaacttgaaaaagtgcgcaccgattcggtgcttttttatttgcttt

SEQ ID NO: 212 - DNA, 98 bp, synthetic construct
  source 1..98: mol_type = other DNA
gttttagagctgtgtcgaaacacagcaagttaaaataaggtttatccgtattcaacttgaaaaagtgcgcaccgattcggtgcttttttatttgcttt

SEQ ID NO: 213 - protein, 1368 aa, synthetic construct
MQKSYSLGLAIGTNSVGWAVITDDYKVPAKKMKVLGNTDRQTVKKNMIGTLLFDSGETAEARRLKRTARRRYTRRINRIKYLQSIFDDEMSKIDSAFFQRIKDSFLVPDDKNDDRHPIFGNIKDEVDYHKNYPTIYHLRKKLADSDEKADLRLIYLALAHIIKFRGHFLIEGDLDSQNTDVNALFLKLVDTYNLMFEDDKIDTQTIDATVILTEKMSKSRRLENLIAKIPNQKKNTLFGNLISLSLGLTPNFKANFELSEDAKLQISKDSFEEDLDNLLAQIGDQYADLFIAAKNLSDAILLSDILTVKGVNTKAPLSASMVQRFNEHQDDLKLLKKLVKVQLPEKYKEIFDIKDKNGYAGYINGKTSQEDFYKYIKPILSKLKGAESLISKLEREDFLRKQRTFDNGSIPHQIHLNELKSIIRRQEKYYPFLKDKQVRIEKIFTFRIPYFVGPLANGNSSFAWVKRRSNESITPWNFEEVVEQEASAKVFIERMTNFDTYLPEEKVLPKHSLLYEMFTVYNELTKVKYQAEGMRKPEFLSSEEKIEIVSNLFKKERKVTVKQLKENYFNKIRCLDSITISGVEDKFNASLGTYHDLLNIIKNQKILDDEQNQDSLEDIVLTLTLFEDEKMIAKRLSKYESIFEPSILKKLKKRHYTGWGRLSQKLINGIRDKQTGKTILDFLIDDGQANRNFMQLINDPSLDFASIIKGAQEKTIKSEKLEETIANLAGSPAIKKGILQSVKIVDEVVKVMGYEPSNIVIEMARENQSTHRGINNSRERLRKLEEVHKNIGSKILKEHEISNAQLQSDRVYLYLLQDGKDMYTGKDLDFDRLSQYDIDAIIPQSFIKDNSIDNIVLTSQESNRGKSDNVPYIAIVNKMKSYWQHQLKSGAISQRKFDNLTKVERGGLSEYDKAGFIKRQLVETRQITKHVAQILNNRFNNNVDNSSKNKRPVKIITLKSKMVSDFRKEFGFYKIREVNDYHHAHDAYLNAVVGTALLKKYPKLEAEFVYGDYKHYDLASLVVKSDTSLGKATAKMFFYSNIMNFFKKEVRLADGTVITRPQIETNTETGEIVWDKVKDIKTIRKVLSIPQINVVKKTEVQTGGFSKESILPKGDSDKLIPRKNNWDPKKYGGFDSPIIAYSVLVVAKVAKGKSQKTKSVKELVGITIMEQNEFEKDRITFLEKKGYQDIQESLIIKLPKFSLFELENGRKRLLASAKELQKGNELSLPNKYIQFLYLASRYTSFSGKEEDREKHRHFVESHLHYFDEIKDIIADFSRRYILADANLEKILTLYNEKNQFSIEEQATNMLNLFTFTGLGAPATLKFFNVDIDRKRYTSSTEILNSTLIRQSITGLYETRIDLSKIGGD

SEQ ID NO: 214 - DNA, 4104 bp, synthetic construct
  source 1..4104: mol_type = other DNA
atgcaaaagagctactctctcgggttagcaatcggaacaaatagtgtgggatgggcggtgattacggacgattataaggtgccagccaaaaagatgaaggttcttggcaatacggaccggcagacggtgaagaagaacatgattggcactctgctgtttgatagtggagaaaccgctgaggcccggagactcaaaaggactgctaggcgacggtatacgcggcgtattaaccgcattaaatatcttcagtctatatttgatgatgagatgtcaaagatcgacagcgcgttttttcagcgaattaaagattccttccttgtcccagatgacaagaatgacgatagacatccgatttttggtaacattaaggacgaggttgactaccataagaactatccgacaatttatcacctgcgcaagaagctggcagactccgacgagaaggcagaccttagactgatttacctcgctctggctcacatcataaaatttcgaggacacttcttgatagaaggagatctcgacagccagaatactgatgttaacgccctgttcctgaaattagtcgacacctacaacctcatgtttgaggatgacaaaatcgatacgcagactattgacgcaacagtgattttaactgagaagatgagtaagtcacggcgacttgagaacttgatagccaagatacctaatcaaaagaagaataccctcttcggaaatctgatttcactcagtcttggcctgacacctaactttaaagctaattttgaattgagcgaggacgcgaagcttcaaatctctaaggactccttcgaagaagatttggataacctcctcgcccagatcggtgaccaatacgctgacctgtttatagcagcgaagaatttgtctgacgctatcctcctgtctgatatccttactgtgaagggcgtgaatacaaaggcacccttatccgccagtatggtccagcggttcaacgaacatcaagacgacctgaagttgctcaaaaaactcgtgaaggtgcaactgcccgagaaatacaaagaaattttcgacattaaagacaaaaatgggtacgctgggtatattaacggtaagacatcccaggaggacttttacaaatatatcaagcctatcttaagcaagctgaaaggggcggagtcccttatctctaaattggagagagaagactttttgcggaagcagagaaccttcgataatggatccattccccaccagattcacttgaatgagctcaaatccatcatccgacgacaggagaagtattatccctttctgaaggataaacaggtgcggattgaaaagatcttcacctttagaataccatattttgttggaccattggctaacgggaactcttcatttgcttgggttaagcgaagatctaacgaatctattacaccatggaactttgaggaagtcgttgagcaggaggccagcgccaaggtcttcatagagcggatgactaattttgatacctacctgccagaggagaaggtccttcccaagcactctttgctctatgaaatgttcactgtatacaacgaactgactaaagtaaagtatcaggccgagggcatgagaaagcccgaattcttgagttcagaagaaaagattgagattgtgtccaacctgtttaagaaggagagaaaggtgacagtcaagcagcttaaggaaaattatttcaataagataagatgtcttgactcaatcaccatcagtggggttgaagacaagttcaacgcatcactgggtacttaccacgatttacttaacattattaagaaccagaagattctggacgatgagcagaaccaggactccctcgaggatattgtgttgactctgacactgttcgaggacgaaaaaatgatcgcgaagaggctgtcaaagtatgaatccattttcgagcccagcattttgaagaaattaaaaaagcgccactatactggttggggccgtttatcccagaagctcatcaacggcatccgtgataaacagaccggaaagaccatcctggacttcctgatcgacgatggccaggcgaatcgaaatttcatgcaattgattaacgatccctctctggactttgcgtcaataatcaagggggcccaggaaaagacgataaagagcgagaagctcgaagagaccatcgctaatctcgccggatctcccgctatcaagaaaggcatcttacagtctgtgaagattgtagatgaagtggtgaaagtgatgggctatgaacctagcaacattgtcatagaaatggccagggaaaatcagtcaacccaccgaggcataaataactctagggaacgattacgaaagctggaggaggtccacaagaacattggctccaagatcttgaaagagcacgaaattagcaatgcccaactccagagtgaccgagtgtacttgtatctgttgcaggatggaaaagatatgtacaccggtaaggacctcgatttcgatcggctctctcagtacgatattgatgcaatcataccacagtcctttattaaggacaacagtattgataatatcgtcctgacatctcaggaaagcaatagaggaaagtcagataatgtgccctacattgcaatcgtgaataagatgaaatcatactggcaacaccagctgaaatctggggctatcagccagcggaaatttgataatttaactaaggtggagcggggcggcctcagcgagtatgataaggcaggttttatcaaacgtcagctcgttgagacacgtcagataacaaagcacgtggcacaaatccttaataatagattcaacaacaacgtcgataacagtagcaagaacaaaagacctgtcaagataatcacattaaaatctaaaatggtgtctgatttccgtaaggaattcggcttctataaaattagggaggtaaatgactatcatcacgcccacgacgcctacctcaacgccgttgtcgggacagccctgttgaaaaaatatccaaagctggaggcagaattcgtgtacggcgattacaagcactatgacttggcctcactggttgtcaagagcgacactagtctgggcaaagccactgcaaaaatgtttttttattctaatatcatgaacttcttcaaaaaggaggtcagactggcagatggcaccgtgatcacaagacctcagatagagactaatacggaaactggcgagatcgtgtgggataaggtaaaggacattaaaacaattaggaaggtgctgtctataccccagatcaacgtggttaaaaagactgaagtccaaactgggggtttctcaaaggaaagcatcctgcccaagggcgatagcgataagcttattcctagaaagaacaattgggatccaaagaagtatggtggctttgattctccgatcattgcctattctgtcttagtggtcgcaaaagtggcgaagggcaaaagccagaagacaaagagtgtcaaagaacttgtcggaattactatcatggaacagaacgagttcgaaaaggatcggattacattccttgagaaaaaaggataccaggatattcaggaatcactgatcattaagctgcccaagttcagcttgtttgagcttgaaaacgggagaaagcgtctgctcgccagcgcaaaagagctccagaagggaaatgagctgtcattgccaaacaagtacatccaatttttgtatctcgcctccagatatactagctttagcggcaaggaggaagatagagagaagcacagacacttcgtggaatctcacctgcactactttgatgagattaaagacataattgccgatttttctcgacgctatattctggcagatgcgaaccttgaaaaaattctcacgctgtacaatgagaaaaatcagttctcaattgaagagcaggctaccaacatgctgaacctcttcaccttcacgggactgggagcccctgccaccctgaaatttttcaacgtggacattgatcggaagcgatacacttcctccaccgagattctgaatagtaccctcattagacagagtattaccggactctacgagacaaggattgacctctccaaaattggcggggac

SEQ ID NO: 215 - RNA, 97 nt, synthetic construct
  source 1..97: mol_type = other RNA
gttttagagctatgttattcgaaaacaacatagcaagttaaaataaggcttgtccgtaatcaacctttcaaggtgaacaccgattcggtgttttttt

SEQ ID NO: 216 - DNA, 97 bp, synthetic construct
  source 1..97: mol_type = other DNA
gttttagagctatgttattcgaaaacaacatagcaagttaaaataaggcttgtccgtaatcaacctttcaaggtgaacaccgattcggtgttttttt

SEQ ID NO: 217 - protein, 1460 aa, synthetic construct
MNKPYSIGLAIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDAIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMGKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGEDTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 218 - protein, 1213 aa, synthetic construct
MTNGKILGLAIGIASVGVGIIEAKTGKVVHANSRLFSAANAENNAERRGFRGSRRLNRRKKHRVKRVRDLFEKYEIVTDFRNLNLNPYELRVKGLTEQLTNEELFAALRTISKRRGISYLDDAEDDSTGSTDYAKSIDENRRLLKTKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCSFYPDEYRASKASYTAQEYNFLNDLNNLKVPTETGKLSTEQKEALVEFAKSTATLGPAKLLKEIAKILDCKVDEIKGYREDDKGKPDLHTFEPYRKLKFNLDSVNIDDLSREVLDKLADILTLNTEREGIEDAIRHNLPNQFTEGQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNGTDKLPDEVFHGNKQLETKIRLWYQQGERCLYSGKPIPIQELVHNSNNFEIDAILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKISVIRGQFTSQLRRKWKIDKSRETYHHHAVDALIIAASSQLKLWEKQDNPMFVDYGNNQVVDKQTGEILSVSDDEYKELVFQPPYQGFVNMISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLVCKYSKKGKGTPIKSLKYYDKKLGNCIDITPEGSKNEVVLQSLNPWRADVYFNPETLKYELLGLKYSDLSFEKGTGKYHISQEKYDVIKEKEGIGKKSEFKFTLYRNDLILIKDTASGEQEIYRFLSRTMPNVKHYAELKPYDKEKFDNVQELVEALGEADKVGRCIKGLNKSNLSIYKVRTDVLGNKYFVKKEGDKPKLDFKNNKKTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 219 - protein, 1451 aa, synthetic construct
MRKPYSIGLAIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADRDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLNGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKDRKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKHEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDAIIPQSFIKDNSIDNIVLTTQASNRGKSDNVPNIEIVNKMKSFWYKQLKNGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESILPKGNSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDEIMQIIVEYSNRYILADSNLIKIQNLYKEKDNFSIEEQAINMLNLFTFTDLGAPAAFKFFNGDIDRKRYSSTNEIINSTLIYQSPTGLYETRIDLSKLGGKTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 220 - protein, 1212 aa, synthetic construct
MSNGKILGLAIGVASVGVGIIDAKTGNVIHANSRLFSAANAENNAERRGFRGARRLTRRKKHRVKRVRDLFEKYDISTDFRNLNLNPYELRVKGLTEQLTNEELFAALRTIAKRRGISYLDDAEDDSTGSSDYAKSIDENRRLLKTKTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKKSKALGASKLLKEIAKIVDCSVDDIKGYRVDNKDKPDLHTFEPYRKLKFNLSSIDIDELSRETLDKLADILTLNTEREGIEDTIKRNLPSQFTEEQISEIVQIRKNQSSAFNKGWHSFSAKLMNELIPELYVTSEEQMTILTRLEKFKVNKKSSKNTKTIDEKEITDEIYNPVVAKSVRQTIKIINAAVKKYGDFDKIVIEMPRDKNAEDEKKFIDKKEKENKKEKDDSLKRAAFLYNGTDNLPDGVFHGNKELKTKIRLWYQQGERCLYSGKLISIHDLVHNSNKFEIDAILPLSLSFDDSLANKVLVYAWTNQEKGQKTPYQVIDSMDAAWSFREMKDYVLKQKRLGKKKREYLLTTENIDKIEVKKKFIERNLVDTRYASRVVLNSLQTALKELGKDTKVSVVRGQFTSQLRRKWNIDKSRETYHHHAVDALIIAASSQLKLWQKQENPMFESYGENQVVNKETGEILSISDDKYKELVFQPPYQGFVNTISSKGFEDEILFSYQVDSKFNRKVSDATIYSTRKAKLGKDKKDETYVLGKIKDIYSQDGFDTFIKRYKKDKTQFLMYQKDPLTWENVIEVILRDYPSEKLSEDGKKTVKCNPFEEYRRENGLICKYSKKGNGTPIKSLKYYDKKLGNCIDITPEKSKNRVVLRQISPWRADIYFNLETLKYELMGLKYSDLSFEKGTGKYHISQEKYDAIREKEGIGKKSEFKFTLYRNDLILIKDTLNNCERMLRFGSKNDTSKHYVELKPLEKGTFDSEEEILPVLGKVAKSGQFIKGLNKPNISIYKVRTDVLGNKFFIKKEGDKPKLDFKNNNKTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 221 - protein, 1428 aa, synthetic construct
MKKPYSIGLAIGTNSVGWAVVTDDYKVPAKKMKVLGNTDKSHIKKNLLGALLFDSGNTAADRRLKRTARRRYTRRRNRILYLQEIFAEEMSKVDDSFFHRLEDSFLVTEDKRGERHPIFGNLEEEVKYYENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEDLEELLGKIGDDYADLFTLAKNLYDAILLSGILTADDSSTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKILTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDAIIPQAFIKDNSIDNRVLTSSKENRGKSDDVPSKDVVRKMKPYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHEENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLSKLGGDTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 222 - protein, 1451 aa, synthetic construct
MQKSYSLGLAIGTNSVGWAVITDDYKVPAKKMKVLGNTDRQTVKKNMIGTLLFDSGETAEARRLKRTARRRYTRRINRIKYLQSIFDDEMSKIDSAFFQRIKDSFLVPDDKNDDRHPIFGNIKDEVDYHKNYPTIYHLRKKLADSDEKADLRLIYLALAHIIKFRGHFLIEGDLDSQNTDVNALFLKLVDTYNLMFEDDKIDTQTIDATVILTEKMSKSRRLENLIAKIPNQKKNTLFGNLISLSLGLTPNFKANFELSEDAKLQISKDSFEEDLDNLLAQIGDQYADLFIAAKNLSDAILLSDILTVKGVNTKAPLSASMVQRFNEHQDDLKLLKKLVKVQLPEKYKEIFDIKDKNGYAGYINGKTSQEDFYKYIKPILSKLKGAESLISKLEREDFLRKQRTFDNGSIPHQIHLNELKSIIRRQEKYYPFLKDKQVRIEKIFTFRIPYFVGPLANGNSSFAWVKRRSNESITPWNFEEVVEQEASAKVFIERMTNFDTYLPEEKVLPKHSLLYEMFTVYNELTKVKYQAEGMRKPEFLSSEEKIEIVSNLFKKERKVTVKQLKENYFNKIRCLDSITISGVEDKFNASLGTYHDLLNIIKNQKILDDEQNQDSLEDIVLTLTLFEDEKMIAKRLSKYESIFEPSILKKLKKRHYTGWGRLSQKLINGIRDKQTGKTILDFLIDDGQANRNFMQLINDPSLDFASIIKGAQEKTIKSEKLEETIANLAGSPAIKKGILQSVKIVDEVVKVMGYEPSNIVIEMARENQSTHRGINNSRERLRKLEEVHKNIGSKILKEHEISNAQLQSDRVYLYLLQDGKDMYTGKDLDFDRLSQYDIDAIIPQSFIKDNSIDNIVLTSQESNRGKSDNVPYIAIVNKMKSYWQHQLKSGAISQRKFDNLTKVERGGLSEYDKAGFIKRQLVETRQITKHVAQILNNRFNNNVDNSSKNKRPVKIITLKSKMVSDFRKEFGFYKIREVNDYHHAHDAYLNAVVGTALLKKYPKLEAEFVYGDYKHYDLASLVVKSDTSLGKATAKMFFYSNIMNFFKKEVRLADGTVITRPQIETNTETGEIVWDKVKDIKTIRKVLSIPQINVVKKTEVQTGGFSKESILPKGDSDKLIPRKNNWDPKKYGGFDSPIIAYSVLVVAKVAKGKSQKTKSVKELVGITIMEQNEFEKDRITFLEKKGYQDIQESLIIKLPKFSLFELENGRKRLLASAKELQKGNELSLPNKYIQFLYLASRYTSFSGKEEDREKHRHFVESHLHYFDEIKDIIADFSRRYILADANLEKILTLYNEKNQFSIEEQATNMLNLFTFTGLGAPATLKFFNVDIDRKRYTSSTEILNSTLIRQSITGLYETRIDLSKIGGDTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 223 - protein, 1140 aa, Streptococcus parasanguinis
MNGLVLGLDIGIASVGVGILKKDIGEIIHTNSRLFSAATADSNIERRGHRGGKRLTRRKKHRSIRLHDLFEDFGLLTDFSKVSINLNPYQLRVQGLDNQLTNEELFIALKNIVKRRGISYLDDASEDGGTVSSDYGKAVEENRKLLAEQTPGQIQLDRFEKYGQVRGDFNVVENGEKRRLINVFTTSAYSKEAERILRKQQEFNKKITDEFIEDYLTILTGKRKYYHGPGNEKSRTDYGRYTTKKDPEGKYITLDNIFGILIGKCTFYPDEYRASKASYTAQEFNLLNDLNNLTVPTETKKLSEEQKKTIIKYAKTAKTLGASTLLKYIAKLIGASVDQIHGYRIDPNKKPEMHTFETYRKMQSLETISVEELPRKVLDELAHILTLNTEREGIEEAINATLKDTFSQDQVLELVQFRKNNSSLFSKGWHSFSLKLMMELIPELYETSEEQMTILTRLGKQKSKETSKRTKYIDEKELTEEIYNPVVAKSVRQAIKIINEATKKYGIFDNIVIEMARENNEEDAKKEYIKRQKANLDEKNAAMEKAAFQYNGKKELPDNVFHGHKELATKIRLWHQQGEKCLYTGKNIPISDLIQNQYKYEIDHILPLSLSFDDSLSNKVLVLATANQEKGQRTPFQALDSMDDAWSYREFKSYVKDSKLLGNKKKEYLLTEEDISKIEVKQKFIERNLVDTRYSSRVVLNALQDFYKEHQFDTTISVVRGQFTSQLRRKWGLEKSRETYHHHAVDALIIAASSQLRLWKKQNNPLISYTEGQFVDQVTGEIISLSDDEYKELVFKAPYDHFVDTLKSKKFEDSILFSYQVDSKYNRKISDATIYATRKAKLDKENKEYTYTLGKIKDIYALGTKSPSKTGFYKFLDLYNKDKSQFLMFQKDRKTWDEVIEKIIEQYRPFKEYDENGKEVDFNPFEKYRIENGPIRKYSKKGNGPEIKSLKYYDNLLGKFVDITPSESKNPVALLSLNPWRTDVYYNTETSKYEFLGLKYADLCFEKGGAYGISEVKYNKIREKEGIGKESEFKFTLYKNDLILIKDTETNCQQIFRFWSRTGKDNPKSFEKHKIELKPYEKARFEKGEELEVLGKVPPSSNQLQKNMQIENLSIYKVKTDVLGNKHFIKKEGEEPKLKF

SEQ ID NO: 224 - DNA, 3423 bp, Streptococcus parasanguinis
  source 1..3423: mol_type = genomic DNA
atgaatggtttagttttgggtttggatattggtattgcttcggttggagtaggtattctaaaaaaagatattggtgaaattattcacactaattctcgtctgttttcagctgcaacggctgacagtaatattgaaagaagaggacataggggaggtaaaagattaactcgccggaagaaacatcgtagtattcgccttcatgatttatttgaagactttggtttgttaactgatttttctaaggtgtccattaatctaaatccttatcaactccgtgtacaagggttggataatcaattaactaatgaagagttgtttattgctttaaagaatattgtgaagagacgtggtattagctatttggatgatgcttctgaggatggcggtacagtatcgtctgattacggtaaggcagttgaagaaaatagaaaattacttgctgaacaaactcccggacaaattcaactagatcgtttcgaaaaatatggtcaggttagaggagatttcaatgttgtagaaaatggtgaaaagcgcagattaatcaatgtttttacaacatctgcttatagcaaagaagctgaaagaattcttagaaagcaacaagaattcaataaaaagattacagatgagtttatagaggattatctaacaatccttacgggaaagagaaaatactaccatggaccaggtaatgaaaagtcacgcaccgattatggaaggtatactactaagaaagatcctgaaggtaagtacataaccttagataatatttttggtattttaattggtaaatgtacattttatcctgatgaatatagagcttcaaaagcttcctatactgctcaagagttcaacttactaaatgatttaaataatttaactgttccaacagaaaccaagaaactgagtgaggaacaaaagaagacaattatcaagtatgcgaagacagctaaaacattaggagcttcaacactattgaagtacatcgctaaattaattggtgcctcagttgatcagatacatggataccgtattgatcccaataaaaaacctgagatgcatacttttgaaacctatcggaaaatgcaatcattagaaacaatcagcgtggaagaattaccgagaaaggtcttagatgagcttgcccatattctaactttaaatacagagcgagagggaatagaagaagcaattaacgcgacgctaaaagatacatttagtcaggatcaagtacttgaattggttcaatttagaaaaaataatagcagtctatttagtaagggatggcatagtttttctctaaaactcatgatggagttgattccagaattgtatgagacttcggaagagcaaatgacaattcttactcgattaggcaaacaaaaatctaaagagacatcaaaacgaaccaagtatattgatgaaaaagaattaacagaagaaatctataaccctgtggtagcaaaatctgtcagacaagctataaagattatcaatgaagcaactaaaaaatatggtatctttgataatatcgttattgaaatggcacgtgaaaataatgaggaagatgcaaagaaagagtatatcaagcgtcaaaaagcaaatctagacgagaaaaatgcagctatggaaaaagcggctttccaatacaatgggaaaaaagaattaccggataatgtttttcatgggcataaggaattagctactaagattcgtttatggcatcagcaaggagaaaagtgtctttacactggtaagaatataccaatttcagatttgattcaaaatcagtataagtatgaaattgatcatattttacccttgtctctttcttttgatgatagtttatccaataaagtattggtacttgctacagcgaaccaagaaaagggacaacgtacaccgtttcaagctttagatagcatggatgatgcctggtcttaccgtgagtttaaatcatatgtaaaagattctaaattattaggtaataaaaagaaggaatatcttttaacggaagaagacattagtaagattgaggtgaaacagaaatttattgaacgaaatttagtagatactcgttattcttctcgcgttgttctaaatgctctacaagatttttataaggagcatcaattcgatacaactatttcagtggtacgtggacaatttacttcgcaactcagaagaaagtggggacttgaaaaatctcgtgagacctatcaccatcatgctgtagatgctttgatcattgctgcatctagtcaattaagattatggaaaaaacaaaataatcctttgatctcttatacagaaggtcaattcgtggatcaagtaactggtgaaattatttcattgagtgacgatgaatataaagagttagtatttaaagcaccatatgatcattttgttgatacattgaagtccaagaaatttgaggatagcattctattttcttatcaagtagattcaaaatataaccgaaagatttcagatgcaacaatttacgcaacaagaaaagcaaaattagataaagagaacaaagaatacacttatactttaggaaaaataaaagatatctatgctttagggacaaaaagtccttctaaaacgggattttataaatttttagatttatacaataaggataaatcccaattcttgatgtttcagaaggatagaaaaacttgggatgaagtgatagagaagataatagaacaatatcgaccatttaaagaatacgatgaaaatggaaaagaagttgatttcaatccttttgaaaaatatagaattgaaaatggtcctattcgcaaatatagtaaaaaaggcaatggtccagaaattaaaagtctaaaatactacgataatcttttagggaaatttgttgatataactccttcagaaagtaagaatcctgtagctttgctttctttgaatccttggagaacagatgtatactacaatacagaaacaagcaaatatgaattcttaggattaaagtatgcagatttgtgtttcgaaaaaggtggtgcttacggaatttcagaagttaaatataataaaataagagagaaagaaggaattggtaaggaatctgaatttaagtttacactatataagaatgatctaattttaattaaggatactgaaacaaattgccaacaaatctttagattttggtctcggacagggaaggataatccaaaaagctttgaaaagcataaaatagaattaaaaccttatgaaaaagcaagatttgagaaaggagaggaacttgaggtattaggaaaggtaccaccttcttctaatcaattacaaaaaaatatgcaaatagagaatctttctatttataaagttaaaacagatgttttgggtaataaacatttcatcaaaaaagagggtgaagaaccaaaactcaaattttaa

SEQ ID NO: 225 - protein, 1140 aa, synthetic construct
MNGLVLGLAIGIASVGVGILKKDIGEIIHTNSRLFSAATADSNIERRGHRGGKRLTRRKKHRSIRLHDLFEDFGLLTDFSKVSINLNPYQLRVQGLDNQLTNEELFIALKNIVKRRGISYLDDASEDGGTVSSDYGKAVEENRKLLAEQTPGQIQLDRFEKYGQVRGDFNVVENGEKRRLINVFTTSAYSKEAERILRKQQEFNKKITDEFIEDYLTILTGKRKYYHGPGNEKSRTDYGRYTTKKDPEGKYITLDNIFGILIGKCTFYPDEYRASKASYTAQEFNLLNDLNNLTVPTETKKLSEEQKKTIIKYAKTAKTLGASTLLKYIAKLIGASVDQIHGYRIDPNKKPEMHTFETYRKMQSLETISVEELPRKVLDELAHILTLNTEREGIEEAINATLKDTFSQDQVLELVQFRKNNSSLFSKGWHSFSLKLMMELIPELYETSEEQMTILTRLGKQKSKETSKRTKYIDEKELTEEIYNPVVAKSVRQAIKIINEATKKYGIFDNIVIEMARENNEEDAKKEYIKRQKANLDEKNAAMEKAAFQYNGKKELPDNVFHGHKELATKIRLWHQQGEKCLYTGKNIPISDLIQNQYKYEIDAILPLSLSFDDSLSNKVLVLATANQEKGQRTPFQALDSMDDAWSYREFKSYVKDSKLLGNKKKEYLLTEEDISKIEVKQKFIERNLVDTRYSSRVVLNALQDFYKEHQFDTTISVVRGQFTSQLRRKWGLEKSRETYHHHAVDALIIAASSQLRLWKKQNNPLISYTEGQFVDQVTGEIISLSDDEYKELVFKAPYDHFVDTLKSKKFEDSILFSYQVDSKYNRKISDATIYATRKAKLDKENKEYTYTLGKIKDIYALGTKSPSKTGFYKFLDLYNKDKSQFLMFQKDRKTWDEVIEKIIEQYRPFKEYDENGKEVDFNPFEKYRIENGPIRKYSKKGNGPEIKSLKYYDNLLGKFVDITPSESKNPVALLSLNPWRTDVYYNTETSKYEFLGLKYADLCFEKGGAYGISEVKYNKIREKEGIGKESEFKFTLYKNDLILIKDTETNCQQIFRFWSRTGKDNPKSFEKHKIELKPYEKARFEKGEELEVLGKVPPSSNQLQKNMQIENLSIYKVKTDVLGNKHFIKKEGEEPKLKF

SEQ ID NO: 226 - DNA, 3420 bp, synthetic construct
  source 1..3420: mol_type = other DNA
atgaacggactggttttgggtcttgccatcgggatcgctagcgttggggtgggcatcctgaagaaggacataggagaaatcattcataccaattctagactgttttcagctgccacagccgactctaatattgaacgacgaggacatcgtggcggcaagaggctgacaagacgaaaaaaacaccgaagcatacgacttcacgatcttttcgaggattttggactgctgacggacttttcaaaagtttccatcaacttgaatccgtaccagttacgcgtacagggtctggacaaccagctgacaaacgaggagctgtttatcgctcttaagaatatcgtgaagcgccgggggattagctacttagacgatgcctctgaagacggcggaaccgtgtcttctgattatgggaaggctgtcgaagaaaatagaaaactcttagccgaacagactcctgggcagatccagctggacagattcgaaaagtacggccaagtccgaggcgacttcaatgtcgtggagaacggtgagaaacgacgtctgattaacgtctttacaactagcgcctattccaaggaggccgagaggatactgaggaagcagcaggagttcaacaagaaaataacggatgagttcatcgaggattacctgaccattcttactggaaaaagaaaatattaccatggtcctggaaacgaaaagtcccggaccgattacgggcggtacacaaccaaaaaggacccagagggcaaatacatcaccctcgataatattttcgggatcctcatcggtaaatgcactttttaccccgatgagtatcgcgcgtctaaagcttcatataccgcacaggagttcaatctgctcaacgacctgaataacctgaccgtgcctaccgaaaccaaaaaactgtcagaggagcagaagaagacgataataaaatacgccaaaacggctaagacccttggcgcttctactctgctgaagtatatagccaaactgatcggtgcttccgttgaccagattcacgggtatagaatcgacccaaataaaaagcccgaaatgcacaccttcgagacgtaccggaaaatgcaatccctggagacgatctcagtggaggaactgcctcgcaaagtgcttgacgaactcgcccatattctgacattgaacactgagcgcgaaggcatcgaggaggctattaatgccaccttgaaagatacgtttagtcaggaccaggtcctcgaactcgtgcagttccgcaaaaataactcttccttattctcaaagggatggcatagtttcagcctgaaactgatgatggaactgattcctgaactctatgagactagtgaagagcagatgactatactgactcgtctggggaaacagaagtctaaggagacaagtaaacgaactaagtacattgatgaaaaggagctgacagaagagatttataatccagtcgtggctaaatccgtccgtcaggctattaagatcattaacgaggcaacgaaaaagtacggaatcttcgataacattgtgatcgaaatggcccgtgagaacaatgaagaagatgctaagaaggagtacatcaagcggcagaaggcaaacttggatgagaagaacgccgcaatggagaaagctgcttttcaatacaacggtaagaaggaactcccggataacgtcttccacggccataaggagctcgccacaaaaatacggttgtggcaccagcagggggaaaagtgcctctacactggaaaaaatatccctatctccgaccttattcaaaatcagtacaagtatgaaatcgacgccatcctcccactgtccctcagtttcgacgatagcctgtccaacaaggtcctcgtgctggctaccgccaatcaggaaaagggccaaagaactccttttcaagctctcgattcaatggatgacgcctggagctatcgggagttcaaatcctatgtgaaggattctaaactcttggggaataaaaagaaggaatacttgttaacagaggaggatatcagtaagatcgaggtgaaacagaagtttatcgaacggaatttagttgacacaaggtactcaagtcgcgtggttcttaacgcccttcaggacttctacaaggaacaccagttcgataccaccattagcgtggttagaggacaatttacatcccagctgcggcggaaatggggactggagaagagccgagaaacctaccatcaccacgccgtggacgcgctgattattgccgccagtagccagctccgcctctggaaaaaacagaacaatcctcttatcagttacaccgaaggccagtttgtggatcaggtgaccggcgagattatatccctttctgacgacgaatacaaagagctggttttcaaagctccatatgaccactttgttgacacactgaagtcaaagaagtttgaggattctatcctcttttcataccaggtggattctaagtacaaccggaagattagcgacgcaaccatatatgcaactcgtaaagccaaattggacaaggagaataaagagtatacttacactctgggtaaaatcaaagatatctatgctcttggcacgaaaagcccttctaaaaccggcttttacaagtttctggacctgtataataaagacaagtctcagttcctgatgtttcagaaagatcgcaagacctgggacgaggtgattgaaaagatcattgagcaataccggccttttaaagaatacgacgaaaacggaaaagaagtagacttcaacccttttgaaaagtaccggatcgaaaatggccccatccgaaaatattccaaaaagggcaatggcccagaaataaagagtctcaagtattacgataacttactgggtaagtttgtggatatcacaccttcagagtccaagaaccccgtagctctgctgtccctgaacccgtggaggactgacgtctactacaacaccgaaacatccaagtatgagttccttgggctgaagtatgctgatttgtgcttcgaaaaggggggtgcatatggtatctctgaggtgaaatacaataaaataagagagaaagaaggtatcggaaaggagtctgaattcaaatttaccctttataagaatgacctgattctgatcaaagatacagagacaaactgccaacagatttttcgcttctggtctcgaactggaaaagacaatcctaaatcatttgagaagcataaaattgagcttaaaccgtatgaaaaggctcgtttcgagaagggtgaagaactggaggtgctgggaaaggtgccacctagttcaaaccaactgcagaaaaatatgcaaattgagaacctctccatctacaaggtgaagactgatgtgcttggcaataagcattttatcaaaaaggaaggagaagaacctaagctcaaattt

SEQ ID NO: 227 - protein, 1223 aa, synthetic construct
MNGLVLGLAIGIASVGVGILKKDIGEIIHTNSRLFSAATADSNIERRGHRGGKRLTRRKKHRSIRLHDLFEDFGLLTDFSKVSINLNPYQLRVQGLDNQLTNEELFIALKNIVKRRGISYLDDASEDGGTVSSDYGKAVEENRKLLAEQTPGQIQLDRFEKYGQVRGDFNVVENGEKRRLINVFTTSAYSKEAERILRKQQEFNKKITDEFIEDYLTILTGKRKYYHGPGNEKSRTDYGRYTTKKDPEGKYITLDNIFGILIGKCTFYPDEYRASKASYTAQEFNLLNDLNNLTVPTETKKLSEEQKKTIIKYAKTAKTLGASTLLKYIAKLIGASVDQIHGYRIDPNKKPEMHTFETYRKMQSLETISVEELPRKVLDELAHILTLNTEREGIEEAINATLKDTFSQDQVLELVQFRKNNSSLFSKGWHSFSLKLMMELIPELYETSEEQMTILTRLGKQKSKETSKRTKYIDEKELTEEIYNPVVAKSVRQAIKIINEATKKYGIFDNIVIEMARENNEEDAKKEYIKRQKANLDEKNAAMEKAAFQYNGKKELPDNVFHGHKELATKIRLWHQQGEKCLYTGKNIPISDLIQNQYKYEIDAILPLSLSFDDSLSNKVLVLATANQEKGQRTPFQALDSMDDAWSYREFKSYVKDSKLLGNKKKEYLLTEEDISKIEVKQKFIERNLVDTRYSSRVVLNALQDFYKEHQFDTTISVVRGQFTSQLRRKWGLEKSRETYHHHAVDALIIAASSQLRLWKKQNNPLISYTEGQFVDQVTGEIISLSDDEYKELVFKAPYDHFVDTLKSKKFEDSILFSYQVDSKYNRKISDATIYATRKAKLDKENKEYTYTLGKIKDIYALGTKSPSKTGFYKFLDLYNKDKSQFLMFQKDRKTWDEVIEKIIEQYRPFKEYDENGKEVDFNPFEKYRIENGPIRKYSKKGNGPEIKSLKYYDNLLGKFVDITPSESKNPVALLSLNPWRTDVYYNTETSKYEFLGLKYADLCFEKGGAYGISEVKYNKIREKEGIGKESEFKFTLYKNDLILIKDTETNCQQIFRFWSRTGKDNPKSFEKHKIELKPYEKARFEKGEELEVLGKVPPSSNQLQKNMQIENLSIYKVKTDVLGNKHFIKKEGEEPKLKFTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 228 - DNA, 3669 bp, synthetic construct
  source 1..3669: mol_type = other DNA
atgaacggactggttttgggtcttgccatcgggatcgctagcgttggggtgggcatcctgaagaaggacataggagaaatcattcataccaattctagactgttttcagctgccacagccgactctaatattgaacgacgaggacatcgtggcggcaagaggctgacaagacgaaaaaaacaccgaagcatacgacttcacgatcttttcgaggattttggactgctgacggacttttcaaaagtttccatcaacttgaatccgtaccagttacgcgtacagggtctggacaaccagctgacaaacgaggagctgtttatcgctcttaagaatatcgtgaagcgccgggggattagctacttagacgatgcctctgaagacggcggaaccgtgtcttctgattatgggaaggctgtcgaagaaaatagaaaactcttagccgaacagactcctgggcagatccagctggacagattcgaaaagtacggccaagtccgaggcgacttcaatgtcgtggagaacggtgagaaacgacgtctgattaacgtctttacaactagcgcctattccaaggaggccgagaggatactgaggaagcagcaggagttcaacaagaaaataacggatgagttcatcgaggattacctgaccattcttactggaaaaagaaaatattaccatggtcctggaaacgaaaagtcccggaccgattacgggcggtacacaaccaaaaaggacccagagggcaaatacatcaccctcgataatattttcgggatcctcatcggtaaatgcactttttaccccgatgagtatcgcgcgtctaaagcttcatataccgcacaggagttcaatctgctcaacgacctgaataacctgaccgtgcctaccgaaaccaaaaaactgtcagaggagcagaagaagacgataataaaatacgccaaaacggctaagacccttggcgcttctactctgctgaagtatatagccaaactgatcggtgcttccgttgaccagattcacgggtatagaatcgacccaaataaaaagcccgaaatgcacaccttcgagacgtaccggaaaatgcaatccctggagacgatctcagtggaggaactgcctcgcaaagtgcttgacgaactcgcccatattctgacattgaacactgagcgcgaaggcatcgaggaggctattaatgccaccttgaaagatacgtttagtcaggaccaggtcctcgaactcgtgcagttccgcaaaaataactcttccttattctcaaagggatggcatagtttcagcctgaaactgatgatggaactgattcctgaactctatgagactagtgaagagcagatgactatactgactcgtctggggaaacagaagtctaaggagacaagtaaacgaactaagtacattgatgaaaaggagctgacagaagagatttataatccagtcgtggctaaatccgtccgtcaggctattaagatcattaacgaggcaacgaaaaagtacggaatcttcgataacattgtgatcgaaatggcccgtgagaacaatgaagaagatgctaagaaggagtacatcaagcggcagaaggcaaacttggatgagaagaacgccgcaatggagaaagctgcttttcaatacaacggtaagaaggaactcccggataacgtcttccacggccataaggagctcgccacaaaaatacggttgtggcaccagcagggggaaaagtgcctctacactggaaaaaatatccctatctccgaccttattcaaaatcagtacaagtatgaaatcgacgccatcctcccactgtccctcagtttcgacgatagcctgtccaacaaggtcctcgtgctggctaccgccaatcaggaaaagggccaaagaactccttttcaagctctcgattcaatggatgacgcctggagctatcgggagttcaaatcctatgtgaaggattctaaactcttggggaataaaaagaaggaatacttgttaacagaggaggatatcagtaagatcgaggtgaaacagaagtttatcgaacggaatttagttgacacaaggtactcaagtcgcgtggttcttaacgcccttcaggacttctacaaggaacaccagttcgataccaccattagcgtggttagaggacaatttacatcccagctgcggcggaaatggggactggagaagagccgagaaacctaccatcaccacgccgtggacgcgctgattattgccgccagtagccagctccgcctctggaaaaaacagaacaatcctcttatcagttacaccgaaggccagtttgtggatcaggtgaccggcgagattatatccctttctgacgacgaatacaaagagctggttttcaaagctccatatgaccactttgttgacacactgaagtcaaagaagtttgaggattctatcctcttttcataccaggtggattctaagtacaaccggaagattagcgacgcaaccatatatgcaactcgtaaagccaaattggacaaggagaataaagagtatacttacactctgggtaaaatcaaagatatctatgctcttggcacgaaaagcccttctaaaaccggcttttacaagtttctggacctgtataataaagacaagtctcagttcctgatgtttcagaaagatcgcaagacctgggacgaggtgattgaaaagatcattgagcaataccggccttttaaagaatacgacgaaaacggaaaagaagtagacttcaacccttttgaaaagtaccggatcgaaaatggccccatccgaaaatattccaaaaagggcaatggcccagaaataaagagtctcaagtattacgataacttactgggtaagtttgtggatatcacaccttcagagtccaagaaccccgtagctctgctgtccctgaacccgtggaggactgacgtctactacaacaccgaaacatccaagtatgagttccttgggctgaagtatgctgatttgtgcttcgaaaaggggggtgcatatggtatctctgaggtgaaatacaataaaataagagagaaagaaggtatcggaaaggagtctgaattcaaatttaccctttataagaatgacctgattctgatcaaagatacagagacaaactgccaacagatttttcgcttctggtctcgaactggaaaagacaatcctaaatcatttgagaagcataaaattgagcttaaaccgtatgaaaaggctcgtttcgagaagggtgaagaactggaggtgctgggaaaggtgccacctagttcaaaccaactgcagaaaaatatgcaaattgagaacctctccatctacaaggtgaagactgatgtgcttggcaataagcattttatcaaaaaggaaggagaagaacctaagctcaaatttaccggtcctaagaaaaagcggaaagtggctagcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 229 - protein, 1768 aa, synthetic construct
MNGLVLGLAIGIASVGVGILKKDIGEIIHTNSRLFSAATADSNIERRGHRGGKRLTRRKKHRSIRLHDLFEDFGLLTDFSKVSINLNPYQLRVQGLDNQLTNEELFIALKNIVKRRGISYLDDASEDGGTVSSDYGKAVEENRKLLAEQTPGQIQLDRFEKYGQVRGDFNVVENGEKRRLINVFTTSAYSKEAERILRKQQEFNKKITDEFIEDYLTILTGKRKYYHGPGNEKSRTDYGRYTTKKDPEGKYITLDNIFGILIGKCTFYPDEYRASKASYTAQEFNLLNDLNNLTVPTETKKLSEEQKKTIIKYAKTAKTLGASTLLKYIAKLIGASVDQIHGYRIDPNKKPEMHTFETYRKMQSLETISVEELPRKVLDELAHILTLNTEREGIEEAINATLKDTFSQDQVLELVQFRKNNSSLFSKGWHSFSLKLMMELIPELYETSEEQMTILTRLGKQKSKETSKRTKYIDEKELTEEIYNPVVAKSVRQAIKIINEATKKYGIFDNIVIEMARENNEEDAKKEYIKRQKANLDEKNAAMEKAAFQYNGKKELPDNVFHGHKELATKIRLWHQQGEKCLYTGKNIPISDLIQNQYKYEIDAILPLSLSFDDSLSNKVLVLATANQEKGQRTPFQALDSMDDAWSYREFKSYVKDSKLLGNKKKEYLLTEEDISKIEVKQKFIERNLVDTRYSSRVVLNALQDFYKEHQFDTTISVVRGQFTSQLRRKWGLEKSRETYHHHAVDALIIAASSQLRLWKKQNNPLISYTEGQFVDQVTGEIISLSDDEYKELVFKAPYDHFVDTLKSKKFEDSILFSYQVDSKYNRKISDATIYATRKAKLDKENKEYTYTLGKIKDIYALGTKSPSKTGFYKFLDLYNKDKSQFLMFQKDRKTWDEVIEKIIEQYRPFKEYDENGKEVDFNPFEKYRIENGPIRKYSKKGNGPEIKSLKYYDNLLGKFVDITPSESKNPVALLSLNPWRTDVYYNTETSKYEFLGLKYADLCFEKGGAYGISEVKYNKIREKEGIGKESEFKFTLYKNDLILIKDTETNCQQIFRFWSRTGKDNPKSFEKHKIELKPYEKARFEKGEELEVLGKVPPSSNQLQKNMQIENLSIYKVKTDVLGNKHFIKKEGEEPKLKFTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 230 - DNA, 5304 bp, synthetic construct
  source 1..5304: mol_type = other DNA
atgaacggactggttttgggtcttgccatcgggatcgctagcgttggggtgggcatcctgaagaaggacataggagaaatcattcataccaattctagactgttttcagctgccacagccgactctaatattgaacgacgaggacatcgtggcggcaagaggctgacaagacgaaaaaaacaccgaagcatacgacttcacgatcttttcgaggattttggactgctgacggacttttcaaaagtttccatcaacttgaatccgtaccagttacgcgtacagggtctggacaaccagctgacaaacgaggagctgtttatcgctcttaagaatatcgtgaagcgccgggggattagctacttagacgatgcctctgaagacggcggaaccgtgtcttctgattatgggaaggctgtcgaagaaaatagaaaactcttagccgaacagactcctgggcagatccagctggacagattcgaaaagtacggccaagtccgaggcgacttcaatgtcgtggagaacggtgagaaacgacgtctgattaacgtctttacaactagcgcctattccaaggaggccgagaggatactgaggaagcagcaggagttcaacaagaaaataacggatgagttcatcgaggattacctgaccattcttactggaaaaagaaaatattaccatggtcctggaaacgaaaagtcccggaccgattacgggcggtacacaaccaaaaaggacccagagggcaaatacatcaccctcgataatattttcgggatcctcatcggtaaatgcactttttaccccgatgagtatcgcgcgtctaaagcttcatataccgcacaggagttcaatctgctcaacgacctgaataacctgaccgtgcctaccgaaaccaaaaaactgtcagaggagcagaagaagacgataataaaatacgccaaaacggctaagacccttggcgcttctactctgctgaagtatatagccaaactgatcggtgcttccgttgaccagattcacgggtatagaatcgacccaaataaaaagcccgaaatgcacaccttcgagacgtaccggaaaatgcaatccctggagacgatctcagtggaggaactgcctcgcaaagtgcttgacgaactcgcccatattctgacattgaacactgagcgcgaaggcatcgaggaggctattaatgccaccttgaaagatacgtttagtcaggaccaggtcctcgaactcgtgcagttccgcaaaaataactcttccttattctcaaagggatggcatagtttcagcctgaaactgatgatggaactgattcctgaactctatgagactagtgaagagcagatgactatactgactcgtctggggaaacagaagtctaaggagacaagtaaacgaactaagtacattgatgaaaaggagctgacagaagagatttataatccagtcgtggctaaatccgtccgtcaggctattaagatcattaacgaggcaacgaaaaagtacggaatcttcgataacattgtgatcgaaatggcccgtgagaacaatgaagaagatgctaagaaggagtacatcaagcggcagaaggcaaacttggatgagaagaacgccgcaatggagaaagctgcttttcaatacaacggtaagaaggaactcccggataacgtcttccacggccataaggagctcgccacaaaaatacggttgtggcaccagcagggggaaaagtgcctctacactggaaaaaatatccctatctccgaccttattcaaaatcagtacaagtatgaaatcgacgccatcctcccactgtccctcagtttcgacgatagcctgtccaacaaggtcctcgtgctggctaccgccaatcaggaaaagggccaaagaactccttttcaagctctcgattcaatggatgacgcctggagctatcgggagttcaaatcctatgtgaaggattctaaactcttggggaataaaaagaaggaatacttgttaacagaggaggatatcagtaagatcgaggtgaaacagaagtttatcgaacggaatttagttgacacaaggtactcaagtcgcgtggttcttaacgcccttcaggacttctacaaggaacaccagttcgataccaccattagcgtggttagaggacaatttacatcccagctgcggcggaaatggggactggagaagagccgagaaacctaccatcaccacgccgtggacgcgctgattattgccgccagtagccagctccgcctctggaaaaaacagaacaatcctcttatcagttacaccgaaggccagtttgtggatcaggtgaccggcgagattatatccctttctgacgacgaatacaaagagctggttttcaaagctccatatgaccactttgttgacacactgaagtcaaagaagtttgaggattctatcctcttttcataccaggtggattctaagtacaaccggaagattagcgacgcaaccatatatgcaactcgtaaagccaaattggacaaggagaataaagagtatacttacactctgggtaaaatcaaagatatctatgctcttggcacgaaaagcccttctaaaaccggcttttacaagtttctggacctgtataataaagacaagtctcagttcctgatgtttcagaaagatcgcaagacctgggacgaggtgattgaaaagatcattgagcaataccggccttttaaagaatacgacgaaaacggaaaagaagtagacttcaacccttttgaaaagtaccggatcgaaaatggccccatccgaaaatattccaaaaagggcaatggcccagaaataaagagtctcaagtattacgataacttactgggtaagtttgtggatatcacaccttcagagtccaagaaccccgtagctctgctgtccctgaacccgtggaggactgacgtctactacaacaccgaaacatccaagtatgagttccttgggctgaagtatgctgatttgtgcttcgaaaaggggggtgcatatggtatctctgaggtgaaatacaataaaataagagagaaagaaggtatcggaaaggagtctgaattcaaatttaccctttataagaatgacctgattctgatcaaagatacagagacaaactgccaacagatttttcgcttctggtctcgaactggaaaagacaatcctaaatcatttgagaagcataaaattgagcttaaaccgtatgaaaaggctcgtttcgagaagggtgaagaactggaggtgctgggaaaggtgccacctagttcaaaccaactgcagaaaaatatgcaaattgagaacctctccatctacaaggtgaagactgatgtgcttggcaataagcattttatcaaaaaggaaggagaagaacctaagctcaaatttaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 231 - RNA, 104 nt, synthetic construct
  source 1..104: mol_type = other RNA
gtttttgtactctcaagatttcgaaaaatcttgcagaagctacaaagataaggcttcatgccgaattcaacaccctgtcatttatggcggggtgttttcgtttt

SEQ ID NO: 232 - DNA, 104 bp, synthetic construct
  source 1..104: mol_type = other DNA
gtttttgtactctcaagatttcgaaaaatcttgcagaagctacaaagataaggcttcatgccgaattcaacaccctgtcatttatggcggggtgttttcgtttt

SEQ ID NO: 233 - RNA, 100 nt, synthetic construct
  source 1..100: mol_type = other RNA
gttttagagctatgtcgaaacgtagcaagttaaaataaggctagtccgttatcaacttgaaaaagtggcaccgagtcggtgctttttgtatctttatttt

SEQ ID NO: 234 - DNA, 100 bp, synthetic construct
  source 1..100: mol_type = other DNA
gttttagagctatgtcgaaacgtagcaagttaaaataaggctagtccgttatcaacttgaaaaagtggcaccgagtcggtgctttttgtatctttatttt

SEQ ID NO: 235 - protein, 1377 aa, Streptococcus dysgalactiae
MNKPYSIGLDIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDHIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMGKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGED

SEQ ID NO: 236 - DNA, 4131 bp, Streptococcus dysgalactiae
  source 1..4131: mol_type = genomic DNA
atgaacaagccttattcaataggattagatatagggacaaattctgtggggtggagtataatcaccgacgattacaaggtgcctgcaaagaagatgcgcgtgctcggcaatacagacaaggaatatattaagaagaacctgatcggggccctcctttttgacggtggcaacacagcagctgaccgccgcctcaagaggaccgctcggagacggtatactcgccggcgtaatcggatcctgtatttgcaggaaatttttgctgaagaaatgtctaaggtggatgattcattctttcaccggctcgaagactcctttctggtggaggaagacaagaggggctcaaagtacccaatcttcgccacaatgcaagaagagaaatactaccacgagaagtttcccacaatctatcatctcaggaaagagctggccgataaaaaagagaaggccgatttgcgactggtttacttggccttggcacacatcataaagttccggggacactttctgattgaagacgaccgttttgacgtccgcaacactgatatacagaagcaataccaagcgttccttgagatctttgacaccacatttgaaaacaaccatctgctgagccaaaatgtggacgtggaagccattctgactgataagatctctaaatctgccaaaaaggacagaatccttgcccagtaccccaaccagaagtcaactggcattttcgccgagtttctgaagttgatagttggcaatcaggccgattttaagaagcacttcaatttggaggacaaaacgcctctccaattcgccaaggactcatatgatgaggacctggagaatctgcttggccaaatcggggatgagttcgctgatctttttagcgtggcaaagaagctctatgactctgtactcctgagcggaatcctgacagttaccgatctttcaacaaaggcacccctgagtgcaagcatgattcaacgctacgacgagcaccatgaggatctgaaacatctgaagcagttcgtcaaggcttctctgcctgaaaactatcgggaggtcttcgccgactcatctaaggacggctacgccggatacatcgagggaaagacaaatcaggaggctttctacaagtacctgttgaagctgcttacaaaacaggaggggagcgaatacttcctggagaagatcaaaaacgaggacttcctgcgtaaacagaggactttcgataatggctccattcctcaccaggtgcatctcacggaactgagagctatcattagacgtcagagtgagtattacccatttctgaaggagaaccaagaccgaatcgaaaaaattctgacgttccggatcccttactatgtcggacctttagctagggaaaaaagtgacttcgcctggatgacccgaaagacagatgatagtatcagaccatggaactttgaagacctggtggacaaagagaagagcgccgaggcttttattcacaggatgaccaataatgatctctatctgcctgaagagaaggtgctgcccaaacacagtctcatctacgaaaaatttacagtctataacgaactgacaaaggtccgctttctggctgaaggattcaaggactttcaatttctgaaccggaagcagaaggaaactatctttaactcattgtttaaggaaaagaggaaggttaccgaaaaagacatcatctcctttttaaacaaggtagatgggtacgaagggattgccattaaaggcattgagaaacagtttaacgccagcctttcaacctaccatgatctcaagaagatcctcggaaaagatttccttgacaataccgacaacgaacttatcctggaggatatagtgcagacactcactctgttcgaggacagggaaatgataaagaagtgcctcgacatatataaagacttctttaccgagagtcaactgaaaaagttgtatagaaggcattacaccggttggggccgactgagtgcaaaactcattaacggcatccggaataaggagaatcaaaagactatcctcgattacctcatcgatgacggaagcgcaaacagaaacttcatgcaactcatcaacgatgatgacctgtctttcaaaccaattatagacaaagccaggactgggagccatagtgacaatctgaaggaagtggtgggagagctggcaggcagccccgcaattaagaaggggatcctgcagagcctcaaaattgtcgatgaactcgtgaaggtcatgggctatgaacctgaacagattgttgtagagatggcccgagagaaccagactactgcgaagggacttagccggagcagacaacgactgaccactttgcgagagagtctggcgaacctgaagtctaatattctcgaggaaaaaaagccaaagtacgtgaaggaccaggtggagaatcaccacctgagcgacgacagactctttctgtattatctgcagaacggcagagatatgtatacgaagaaggcactggacatagacaacctgagtcagtatgacatcgatcacattatccctcaggccttcatcaaagacgattcaatcgacaatcgcgtacttgttagcagtgcgaaaaaccggggaaagtctgatgacgtcccatccatcgaaatagtgaaggcaaggaagatgttctggaagaatctgctggatgccaaattaatgtcacaacggaagtacgacaacctgacaaaggcagaaagggggggcttaacaagcgacgataaggcaaggtttatccagaggcagttggtcgagaccaggcaaatcaccaaacacgtcgcccggatcctggatgaacgcttcaacaatgaagtcgacaatggcaaaaaaatctgtaaagtcaagatagtgacactgaagtcaaatctggtgagcaacttccggaaagaattcggcttctataaaattcgcgaagtgaacgactatcaccatgcgcacgacgcttacctgaatgcagtcgtggcgaaagccattttgaccaagtacccccagctggagcctgagtttgtgtacggaatgtaccgacaaaagaagctgagcaagattgtacacgaggataaggaagagaaatactccgaggccactcggaagatgttcttctactctaatctgatgaacatgtttaagagagtggtgaggttggcagacggctccattgttgtaaggccagtgatcgagactgggcgatacatgggcaagacagcgtgggacaagaagaagcatttcgcaaccgtacggaaagtcctgtcctacccgcagaataacattgtgaagaagacagaaatacaaaccggtggtttctcaaaagagtccattttagcccatggcaacagtgacaaattgattccacggaagaccaaagatatttatctggaccctaaaaaatacggcggattcgactcaccgatcgtggcatacagcgtattggtggtggccgatattaagaagggtaaagcccagaaactcaagactgttaccgagctcctgggtatcactataatggagagaagccggtttgagaagaaccctagcgcctttttggaatccaaggggtatctgaacattcgggacgataagctgatgatcttgcctaaatacagcctttttgaactggagaatggacgaaggcgcctgcttgcctcagcgggggaactgcagaaaggcaatgagctggcccttcctacccagttcatgaaatttttgtatctggctagtaggtataacgagtcaaaaggcaagccagaggagatcgaaaagaagcaggaatttgtaaaccagcatgtgtcatactttgatgatatcctgcagttaatcaatgacttcagtaaacgagtcattctcgcagacgccaacttggagaaaattaataagctgtaccaggacaacaaagagaatataccagtcgacgagcttgcaaataacattattaacctgttcacttttacatccctgggggcccctgctgcgttcaaatttttcgacaaaatcgtggatcgaaagcgatatacatccactaaggaagttctgaacagcactctcatccaccagtctatcactggcctttacgaaacgcgtattgacttggggaaactcggagaggac

SEQ ID NO: 237 - protein, 1371 aa, synthetic construct
MDKKYSIGLAIGTNSVGWAVITDDYKVPSKKFKVLGNTDRHSIKKNLIGALLFDSGETAEATRLKRTARRRYTRRKNRIRYLQEIFSSEMSKVDDSFFHRLEESFLVEEDKKHERHPIFGNIVDEVAYHEKYPTIYHLRKKLADSTDKADLRLIYLALAHMIKFRGHFLIEGDLNPDNSDMDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPEFLSGKQKEAIVDLLFKTNRKVTVKQLKEDYFKKIECFDSVEISGVEDRFNASLGTYHDLLKIIKDKDFLDNEENEDILEDIVLTLTLFEDKEMIEERLKKYANLFDDKVMKQLKRRHYTGWGRLSRKLINGIRDKQSGKTILDFLKSDGFANRNFMQLINDDSLTFKEAIQKAQVSGQGHSLHEQIANLAGSPAIKKGILQSVKVVDELVKVMGHKPENIVIEMARENQTTQKGQKNSRERMKRIEEGIKELGSQILKEHPVENTQLQNEKLYLYYLQNGRDMYVDQELDINRLSDYDVDAIVPQSFIKDDSIDNKILTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYTKLESEFVYGDYKVYDVRKMIAKSEQEIGKATAKRFFYSNIMNFFKTEITLANGEIRKRPLIETNEETGEIVWDKGRDFATVRKVLSMPQVNIVKKTEVQTGALTNESIYARGSFDKLISRKHRFESSKYGGFGSPTVTYSVLVVAKSKVQDGKVKKIKTGKELIGMTLLDKLVFEKNPLKFIEDKGYGNVQIDKCIKLPKYSLFEFENGTRRMLASVMANNNSRGDLQKANEMFLPAKLVTLLYHAHKIESSKELEHEAYILDHYNDLYQLLSYIERFASLYVDVEKNISKVKELFSNIESYSISEICSSVINLLTLTASGAPADFKFLGTTIPRKRYGSPQSILSSTLIHQSITGLYETRIDLSQLGGD

SEQ ID NO: 238 - DNA, 4113 bp, synthetic construct
  source 1..4113: mol_type = other DNA
atggataagaagtactccattggactggcaattgggacaaattcagtgggatgggctgtgataacggatgattataaagtgccatctaagaagtttaaagtgctggggaacacagacagacactcaatcaaaaagaatttgattggggccctcctcttcgactcaggtgagaccgctgaagctactcgcctcaagagaacagcgagacggcggtatactcgtagaaagaaccgcattcgctacctgcaagagatatttagcagcgaaatgagtaaggtggacgatagcttcttccacagactggaggagagctttcttgtggaggaggacaagaaacacgagcgccatcccatctttggtaatattgtggacgaggtggcctatcatgagaagtatccaacaatttaccaccttagaaagaagttggcagattccaccgacaaagctgacctccggctgatctaccttgctctcgcacatatgattaaattccggggacacttcttgattgaaggcgaccttaaccccgacaactcagatatggacaagctcttcatccagctcgtacaaacctacaatcagcttttcgaggaaaacccaattaacgcttccagggtcgacgcaaaagcgatactgtctgctcgtcttagtaagtcccggcggctcgagaacttaattgcacagttgcccggcgaaaagcgtaatggactgtttgggaatctcattgccctttcccttggactgactccaaatttcaagtcaaatttcgatctcgctgaggacgcaaaactgcagctgtctaaggacacttacgacgatgacctggacaacctgctggctcagattggcgaccagtacgccgatttattcctcgccgcaaaaaacctttctgatgccatcctgctgagcgatattcttagagttaacagtgagattacaaaagcccccctgagtgcctccatgattaagcgctatgacgaacaccaccaagacttgactctcctgaaagctttagtacggcaacagctccccgagaaatataaggagatctttttcgatcaatccaagaacggatacgcgggatatatagatggaggggctagccaagaggaattttacaagttcatcaaaccaattttagaaaagatggacggaacagaagaattattggccaagctgaatcgggaggatctgctgagaaagcagagaacattcgataacggctccataccccaccagatccacctcggagaattacacgcaattcttagacgccaggaggatttctaccccttcctgaaagacaatcgagagaagattgaaaaaatactgacatttcggatcccctattacgtgggtcctctggcccgagggaatagtcggttcgcctggatgacacgtaagtcagaagagacgattaccccctggaattttgaggaagtggttgataaaggcgccagcgctcagtctttcatcgagcgtatgactaattttgacaaaaacttgcccaacgagaaagtcctccccaaacactccttactttatgagtacttcaccgtctataacgagcttacaaaagttaagtacgtaactgagggtatgaggaaaccagagttcctcagcgggaaacaaaaggaggccattgtggatctgcttttcaaaacaaacaggaaggttaccgtgaaacaattaaaggaggattactttaagaaaatcgagtgcttcgatagcgtcgagatatctggagtagaagacaggttcaacgcgtccctgggtacctaccacgatctgctgaaaataatcaaggacaaggacttcctcgataatgaggaaaatgaagatatcctggaggacatcgtgcttactctgacactgtttgaagacaaagagatgatagaggagaggctgaagaaatatgcaaatcttttcgatgataaagttatgaaacagcttaagcgaaggcattacaccgggtgggggaggctgagccggaagcttatcaatgggatcagggacaagcagagcgggaagactatattggattttctgaagtctgatgggtttgcaaataggaacttcatgcagctcattaatgacgattcactgacatttaaggaggctattcagaaggctcaagtaagtggacaggggcatagcctgcacgaacagattgctaatctcgccggatctccagcaattaagaagggcatcctgcagagtgttaaagttgtggacgagctggtcaaggtgatgggccacaagcctgaaaatatagttattgagatggcgagggaaaaccaaacaactcagaaaggacaaaaaaactcccgcgaacgaatgaaaaggatcgaagagggcattaaagaattgggctcccagattctcaaagaacatcctgttgaaaatacccagctgcagaacgagaagctgtatctgtattatctgcagaacgggagagatatgtacgtcgaccaggagctggacattaaccgattgtctgactacgatgtcgacgcaatcgttccgcaaagcttcataaaggatgattccatcgacaataaaattctcactcggagcgacaaaaatcgaggaaagtctgacaatgtgcccagcgaagaggtggtaaagaagatgaagaactactggagacagcttctgaatgctaaactgattactcaacgtaagttcgacaatctgacaaaggctgaaagggggggtctgagcgagctggataaggctgggttcattaaaaggcagttggtcgaaacccgacaaatcaccaagcatgttgctcagatcttggactcaagaatgaacacaaaatatgatgaaaacgataaactgattagggaggtgaaggtgatcactcttaagagcaagttagtctcagacttcaggaaagattttcagttctataaggtgcgggagattaacaactatcatcatgcccacgacgcgtatctcaacgcggttgtgggaaccgccctgatcaaaaagtacactaagctggagagcgagtttgtttatggagattataaagtgtacgacgtaaggaagatgatcgcgaagtcagagcaggagatcggtaaagctaccgcaaagcgcttcttctacagtaacattatgaacttcttcaagacagagattacgctcgccaatggcgagatacggaagagacccctgattgagactaacgaagaaacaggcgagatcgtttgggacaaaggaagagatttcgctacagtgcggaaagtgctctctatgccccaggtgaatatcgtcaagaagacagaagtgcagaccggagcgttaaccaacgagagcatatatgcacgcggctcctttgataagctgatctccaggaagcacaggttcgagtcctccaagtacgggggcttcggcagcccaactgttacttactccgtcctggtggtggccaaaagcaaagtccaagacgggaaggtcaaaaagatcaagacagggaaagagctgattggcatgacactgttggacaagttggtgttcgagaaaaaccccctgaaatttatagaagacaaggggtacggaaacgtgcagatcgataagtgcattaagctgcctaagtactctttattcgagttcgaaaacggcacccgtcggatgttagcctccgtcatggcgaataataacagcaggggcgacttgcagaaagctaacgaaatgtttctgcctgccaagttggtgacattgctgtatcacgcccacaagattgaatcaagcaaagagctggagcacgaggcatacatccttgatcattacaatgatttgtatcagctcctgtcttacatcgaacggttcgccagcctgtatgtggacgtagagaagaacatatctaaggtaaaggagttgttttccaacatcgaatcctacagcatcagtgagatctgctcctctgtgattaatctcttaactttaacagctagcggggccccggccgactttaaattcttaggtacaacgatcccgcgcaagaggtacggctccccccaatcaattctctccagcacactgattcaccagagcatcaccggcttatatgaaacgaggattgacctgagtcagcttggtggcgac

SEQ ID NO: 239 - protein, 1454 aa, synthetic construct
MDKKYSIGLAIGTNSVGWAVITDDYKVPSKKFKVLGNTDRHSIKKNLIGALLFDSGETAEATRLKRTARRRYTRRKNRIRYLQEIFSSEMSKVDDSFFHRLEESFLVEEDKKHERHPIFGNIVDEVAYHEKYPTIYHLRKKLADSTDKADLRLIYLALAHMIKFRGHFLIEGDLNPDNSDMDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPEFLSGKQKEAIVDLLFKTNRKVTVKQLKEDYFKKIECFDSVEISGVEDRFNASLGTYHDLLKIIKDKDFLDNEENEDILEDIVLTLTLFEDKEMIEERLKKYANLFDDKVMKQLKRRHYTGWGRLSRKLINGIRDKQSGKTILDFLKSDGFANRNFMQLINDDSLTFKEAIQKAQVSGQGHSLHEQIANLAGSPAIKKGILQSVKVVDELVKVMGHKPENIVIEMARENQTTQKGQKNSRERMKRIEEGIKELGSQILKEHPVENTQLQNEKLYLYYLQNGRDMYVDQELDINRLSDYDVDAIVPQSFIKDDSIDNKILTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYTKLESEFVYGDYKVYDVRKMIAKSEQEIGKATAKRFFYSNIMNFFKTEITLANGEIRKRPLIETNEETGEIVWDKGRDFATVRKVLSMPQVNIVKKTEVQTGALTNESIYARGSFDKLISRKHRFESSKYGGFGSPTVTYSVLVVAKSKVQDGKVKKIKTGKELIGMTLLDKLVFEKNPLKFIEDKGYGNVQIDKCIKLPKYSLFEFENGTRRMLASVMANNNSRGDLQKANEMFLPAKLVTLLYHAHKIESSKELEHEAYILDHYNDLYQLLSYIERFASLYVDVEKNISKVKELFSNIESYSISEICSSVINLLTLTASGAPADFKFLGTTIPRKRYGSPQSILSSTLIHQSITGLYETRIDLSQLGGDTGPKKKRKVASMDAKSLTAWSRTLVTFKDVFVDFTREEWKLLDTAQQIVYRNVMLENYKNLVSLGYQLTKPDVILRLEKGEEP

SEQ ID NO: 240 - DNA, 4362 bp, synthetic construct
  source 1..4362: mol_type = other DNA
atggataagaagtactccattggactggcaattgggacaaattcagtgggatgggctgtgataacggatgattataaagtgccatctaagaagtttaaagtgctggggaacacagacagacactcaatcaaaaagaatttgattggggccctcctcttcgactcaggtgagaccgctgaagctactcgcctcaagagaacagcgagacggcggtatactcgtagaaagaaccgcattcgctacctgcaagagatatttagcagcgaaatgagtaaggtggacgatagcttcttccacagactggaggagagctttcttgtggaggaggacaagaaacacgagcgccatcccatctttggtaatattgtggacgaggtggcctatcatgagaagtatccaacaatttaccaccttagaaagaagttggcagattccaccgacaaagctgacctccggctgatctaccttgctctcgcacatatgattaaattccggggacacttcttgattgaaggcgaccttaaccccgacaactcagatatggacaagctcttcatccagctcgtacaaacctacaatcagcttttcgaggaaaacccaattaacgcttccagggtcgacgcaaaagcgatactgtctgctcgtcttagtaagtcccggcggctcgagaacttaattgcacagttgcccggcgaaaagcgtaatggactgtttgggaatctcattgccctttcccttggactgactccaaatttcaagtcaaatttcgatctcgctgaggacgcaaaactgcagctgtctaaggacacttacgacgatgacctggacaacctgctggctcagattggcgaccagtacgccgatttattcctcgccgcaaaaaacctttctgatgccatcctgctgagcgatattcttagagttaacagtgagattacaaaagcccccctgagtgcctccatgattaagcgctatgacgaacaccaccaagacttgactctcctgaaagctttagtacggcaacagctccccgagaaatataaggagatctttttcgatcaatccaagaacggatacgcgggatatatagatggaggggctagccaagaggaattttacaagttcatcaaaccaattttagaaaagatggacggaacagaagaattattggccaagctgaatcgggaggatctgctgagaaagcagagaacattcgataacggctccataccccaccagatccacctcggagaattacacgcaattcttagacgccaggaggatttctaccccttcctgaaagacaatcgagagaagattgaaaaaatactgacatttcggatcccctattacgtgggtcctctggcccgagggaatagtcggttcgcctggatgacacgtaagtcagaagagacgattaccccctggaattttgaggaagtggttgataaaggcgccagcgctcagtctttcatcgagcgtatgactaattttgacaaaaacttgcccaacgagaaagtcctccccaaacactccttactttatgagtacttcaccgtctataacgagcttacaaaagttaagtacgtaactgagggtatgaggaaaccagagttcctcagcgggaaacaaaaggaggccattgtggatctgcttttcaaaacaaacaggaaggttaccgtgaaacaattaaaggaggattactttaagaaaatcgagtgcttcgatagcgtcgagatatctggagtagaagacaggttcaacgcgtccctgggtacctaccacgatctgctgaaaataatcaaggacaaggacttcctcgataatgaggaaaatgaagatatcctggaggacatcgtgcttactctgacactgtttgaagacaaagagatgatagaggagaggctgaagaaatatgcaaatcttttcgatgataaagttatgaaacagcttaagcgaaggcattacaccgggtgggggaggctgagccggaagcttatcaatgggatcagggacaagcagagcgggaagactatattggattttctgaagtctgatgggtttgcaaataggaacttcatgcagctcattaatgacgattcactgacatttaaggaggctattcagaaggctcaagtaagtggacaggggcatagcctgcacgaacagattgctaatctcgccggatctccagcaattaagaagggcatcctgcagagtgttaaagttgtggacgagctggtcaaggtgatgggccacaagcctgaaaatatagttattgagatggcgagggaaaaccaaacaactcagaaaggacaaaaaaactcccgcgaacgaatgaaaaggatcgaagagggcattaaagaattgggctcccagattctcaaagaacatcctgttgaaaatacccagctgcagaacgagaagctgtatctgtattatctgcagaacgggagagatatgtacgtcgaccaggagctggacattaaccgattgtctgactacgatgtcgacgcaatcgttccgcaaagcttcataaaggatgattccatcgacaataaaattctcactcggagcgacaaaaatcgaggaaagtctgacaatgtgcccagcgaagaggtggtaaagaagatgaagaactactggagacagcttctgaatgctaaactgattactcaacgtaagttcgacaatctgacaaaggctgaaagggggggtctgagcgagctggataaggctgggttcattaaaaggcagttggtcgaaacccgacaaatcaccaagcatgttgctcagatcttggactcaagaatgaacacaaaatatgatgaaaacgataaactgattagggaggtgaaggtgatcactcttaagagcaagttagtctcagacttcaggaaagattttcagttctataaggtgcgggagattaacaactatcatcatgcccacgacgcgtatctcaacgcggttgtgggaaccgccctgatcaaaaagtacactaagctggagagcgagtttgtttatggagattataaagtgtacgacgtaaggaagatgatcgcgaagtcagagcaggagatcggtaaagctaccgcaaagcgcttcttctacagtaacattatgaacttcttcaagacagagattacgctcgccaatggcgagatacggaagagacccctgattgagactaacgaagaaacaggcgagatcgtttgggacaaaggaagagatttcgctacagtgcggaaagtgctctctatgccccaggtgaatatcgtcaagaagacagaagtgcagaccggagcgttaaccaacgagagcatatatgcacgcggctcctttgataagctgatctccaggaagcacaggttcgagtcctccaagtacgggggcttcggcagcccaactgttacttactccgtcctggtggtggccaaaagcaaagtccaagacgggaaggtcaaaaagatcaagacagggaaagagctgattggcatgacactgttggacaagttggtgttcgagaaaaaccccctgaaatttatagaagacaaggggtacggaaacgtgcagatcgataagtgcattaagctgcctaagtactctttattcgagttcgaaaacggcacccgtcggatgttagcctccgtcatggcgaataataacagcaggggcgacttgcagaaagctaacgaaatgtttctgcctgccaagttggtgacattgctgtatcacgcccacaagattgaatcaagcaaagagctggagcacgaggcatacatccttgatcattacaatgatttgtatcagctcctgtcttacatcgaacggttcgccagcctgtatgtggacgtagagaagaacatatctaaggtaaaggagttgttttccaacatcgaatcctacagcatcagtgagatctgctcctctgtgattaatctcttaactttaacagctagcggggccccggccgactttaaattcttaggtacaacgatcccgcgcaagaggtacggctccccccaatcaattctctccagcacactgattcaccagagcatcaccggcttatatgaaacgaggattgacctgagtcagcttggtggcgacaccggtcctaagaaaaagcggaaagtggctagcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 241 - protein, 1377 aa, Streptococcus agalactiae; identical to SEQ ID NO: 235

SEQ ID NO: 242 - DNA, 4131 bp, Streptococcus agalactiae; identical to SEQ ID NO: 236
  source 1..4131: mol_type = genomic DNA

SEQ ID NO: 243 - protein, 1132 aa, Streptococcus gallolyticus
MTNGKILGLDIGIASVGVGIIEAKTGKVVHANSRLFSAANAENNAERRGFRGSRRLNRRKKHRVKRVRDLFEKYEIVTDFRNLNLNPYELRVKGLTEQLTNEELFAALRTISKRRGISYLDDAEDDSTGSTDYAKSIDENRRLLKTKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCSFYPDEYRASKASYTAQEYNFLNDLNNLKVPTETGKLSTEQKEALVEFAKSTATLGPAKLLKEIAKILDCKVDEIKGYREDDKGKPDLHTFEPYRKLKFNLDSVNIDDLSREVLDKLADILTLNTEREGIEDAIRHNLPNQFTEGQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNGTDKLPDEVFHGNKQLETKIRLWYQQGERCLYSGKPIPIQELVHNSNNFEIDHILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKISVIRGQFTSQLRRKWKIDKSRETYHHHAVDALIIAASSQLKLWEKQDNPMFVDYGNNQVVDKQTGEILSVSDDEYKELVFQPPYQGFVNMISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLVCKYSKKGKGTPIKSLKYYDKKLGNCIDITPEGSKNEVVLQSLNPWRADVYFNPETLKYELLGLKYSDLSFEKGTGKYHISQEKYDVIKEKEGIGKKSEFKFTLYRNDLILIKDTASGEQEIYRFLSRTMPNVKHYAELKPYDKEKFDNVQELVEALGEADKVGRCIKGLNKSNLSIYKVRTDVLGNKYFVKKEGDKPKLDFKNNKKTG

SEQ ID NO: 244 - DNA, 3396 bp, Streptococcus gallolyticus
  source 1..3396: mol_type = genomic DNA
atgacaaacggcaaaattctgggtctggatatcggaatcgctagcgttggcgtgggaatcattgaagcgaagacaggtaaagtcgtccatgcaaattctcgattgttctccgcagctaacgctgaaaacaatgcggagagaaggggtttcagaggctctaggcggctcaaccggcgcaagaagcacagggtaaaaagagtgcgagatctctttgagaaatatgagatcgtgactgattttagaaacctgaatctgaacccatatgagctgagagtgaaaggacttacggaacagctcactaatgaagagttgttcgccgccctgcggaccatcagcaaacgccgaggaatttcctaccttgatgacgccgaagatgacagtaccggtagcacagattatgccaagagcattgatgagaacaggagactgctgaagactaagacacctggacagatacaattggaacggctcgagaagtacggccagctgaggggtaacttcaccgtttatgacgaaaatggggaggcccatagactgataaatgtgttctcaacttctgactatgaaaaggaggcccggaaaatcctcgagactcaagccgactacaacaagaagattacagccgagtttattgacgattacgtggaaattttaacccagaaaaggaagtattaccacgggccaggaaatgaaaagagccgcaccgactatgggagattcagaacggatggaacaaccttagagaatatctttggaatccttattggtaaatgctctttctatcctgacgagtatcgcgccagcaaagcctcctataccgctcaggagtacaacttcttgaatgatttgaacaatttgaaggttccgacggagactggcaagctgagtaccgagcaaaaggaggcccttgtggaattcgccaagtctactgcaacattaggtcctgctaaacttctgaaggagattgccaaaattttggactgcaaagtcgatgaaatcaaggggtaccgtgaggatgataaagggaaaccagacctgcacacctttgagccctatagaaagttgaaattcaatctggacagcgtcaacattgacgatttgagtcgcgaagtgctggacaagctggcagacattttgacacttaacactgaaagggagggcattgaggatgccatcaggcataacctgcccaaccaatttactgagggccagatctccgaaatcatcaaggtgcgcaaaagccagagcactgctttcaacaaggggtggcacagcttctctgccaagctcatgaacgaattgattcccgagctctatgccacaagcgacgaacagatgactatacttactcggctggagaaatttaaggtcaataaaaaatcctccaaaaacaccaagacgattgacgagaaagaggtcactgatgaaatctacaatccagttgtagccaagtctgtccggcaaacgatcaagatcattaacgctgctgtgaagaaatatggagactttgataagattgtgattgaaatgcctcgcgacaagaatgcggacgatgagaagaagtttatcgataagagaaacaaagaaaataagaaagaaaaggatgatgccctgaagcgggcagcttacctttataatggaaccgataagctgccagatgaggtgtttcacggaaacaagcaacttgaaaccaagattcgcctgtggtaccagcagggagaacggtgtttgtactcaggcaagcctatcccaatccaggagttggtccacaactccaataacttcgaaatcgatcacattctgcccctgtccctgagttttgacgactccctggccaacaaggtgcttgtgtatgcttggaccaaccaagagaagggccagaagacgccctaccaggtgattgattctatggatgcggcgtggtcctttcgcgagatgaaggactatgtgctcaagcaaaaaggcctcggcaaaaagaaacgggattatcttttgaccaccgagaacattgacaagattgaagtgaagaaaaaattcatcgagcgcaacttggtcgataccagatatgcctctagggttgtgctgaactcactgcagtctgctttgagagagctgggtaaagacactaaaattagtgtaatcaggggccagttcacaagtcagcttaggcggaaatggaagatcgacaagtcacgcgagacatatcatcatcacgcagtcgacgcactgataattgcagcttcaagtcagctcaagttgtgggagaaacaggataaccctatgtttgtcgactatggaaacaatcaggtcgtcgataagcagaccggggaaattttaagtgtgtccgatgacgagtataaggagcttgtctttcagccaccgtaccagggctttgtcaacatgattagtagcaagggttttgaggacgaaattttgttcagctaccaggtcgattccaaatacaatagaaaagtatccgacgcaaccatatattctactcgcaaggccaagattggcaaagataagaaggaagagacctatgtattggggaagatcaaagacatttactcacaaaatggattcgacaccttcattaagaagtacaacaaagataagacacagtttttgatgtaccagaaagatccactgacatgggaaaacgtgatcgaagttatactgcgtgactaccccacgactaaaaagagtgaggacggaaaaaacgacgtgaagtgcaacccgtttgaagaataccggagagaaaacggtctggtgtgtaagtactctaagaaaggaaaggggacccctattaaatccctcaaatactacgacaaaaaactcgggaactgcatcgatatcaccccggaaggttccaaaaatgaagtcgtgcttcaatccttgaatccgtggagggcagatgtgtactttaacccagaaaccttgaagtatgaattactgggacttaaatacagtgatctctcatttgaaaagggcactggaaaataccatatctctcaggagaagtacgacgtcattaaggaaaaagaagggatcgggaaaaaatccgagttcaagttcacattgtataggaacgacctgatccttattaaagacacagccagcggtgagcaggagatttaccgatttctgtctagaaccatgcctaacgtcaagcactatgcggagctgaagccctatgacaaagaaaaatttgataacgtccaggaactcgtcgaggcgctgggcgaagccgacaaggtaggccgctgtataaaggggctgaacaaaagcaacctcagcatctataaagttaggacagatgtgctcgggaacaaatacttcgttaagaaggaaggggacaagcccaagctggattttaagaacaataaaaagaccggt

SEQ ID NO: 245 - protein, 1368 aa, Streptococcus iniae
MRKPYSIGLDIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADRDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLNGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKDRKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKHEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDHIIPQSFIKDNSIDNIVLTTQASNRGKSDNVPNIEIVNKMKSFWYKQLKNGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESILPKGNSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDEIMQIIVEYSNRYILADSNLIKIQNLYKEKDNFSIEEQAINMLNLFTFTDLGAPAAFKFFNGDIDRKRYSSTNEIINSTLIYQSPTGLYETRIDLSKLGGK

SEQ ID NO: 246 - DNA, 4104 bp, Streptococcus iniae
  source 1..4104: mol_type = genomic DNA
atgcgcaaaccttactcaattggcctggatatcgggactaattctgttggctgggctgtgattactgatgattacaaggtgccaagtaagaaaatgaggattcagggcacgactgatcggaccagcattaagaagaatctcattggggccctcctgttcgataatggcgagactgccgaggccactcgattaaagagaacaacaaggaggaggtacaccagacggaagtaccgaataaaggaactgcaaaagatcttcagcagcgaaatgaatgagctcgacattgcttttttccctagactgtctgagagttttcttgtgagtgacgacaaagaattcgagaatcatccgatttttggaaaccttaaagatgagataacttatcataacgattaccctactatttatcacttgcgacagacacttgcagaccgtgaccagaaggccgatcttaggctcatttatctcgctctggcccacattattaaatttcgggggcactttttgatcgaaggcaatctggacagtgagaacacggacgtacacgtgctgtttctgaacctggtgaacatatataataacctgttcgaggaagatatagttgaaaccgcatccatagacgctgagaagattcttacctcaaaaacttccaaatccaggcggctcgagaatcttatagctgagattcctaaccagaagcggaacatgttgtttggcaacctcgtgtctctggctctcggcctgacaccaaattttaaaaccaattttgagctgctggaggatgcaaagttacagatctccaaggattcatatgaagaagacctcgacaacttgttggcacagattggggatcagtacgcagatctctttatcgccgctaaaaagctttctgacgcaatattactgtctgacatcatcaccgtgaagggcgcctccactaaagcgcctctttcagcatccatggtgcagagatatgaagagcatcaacaggacctcgctctcctgaagaatctcgtgaaaaaacagattcctgagaagtataaggaaatcttcgataacaaggagaagaatggctatgcaggttatatcgatggcaagacctcccaggaggaattttacaagtacatcaagcccatacttcttaagctcaacggcacagagaagttgatcagcaaacttgagcgggaggacttcctgagaaagcaacgaacattcgacaacggatctattcctcaccagattcacctgaatgagctcaaggcaatcatccggaggcaggagaagttttatccctttctgaaggaaaatcagaagaaaatcgaaaagcttttcacatttaaaattccctattacgtcgggccactcgccaatggccagagtagcttcgcctggctgaagagacagtccaacgagtctatcaccccctggaacttcgaggaagtggtggatcaagaggcctcagcgcgcgccttcatagagaggatgactaacttcgatacctatttacccgaggagaaggttctgccaaagcacagcccactctacgaaatgtttatggtctataatgagctcaccaaggttaagtatcagaccgaggggatgaagaggcccgtctttctctcttccgaagacaaagaagaaatagtgaatctcctgttcaaaaaagaccggaaggtcactgtcaagcagctgaaggaggaatatttctccaaaatgaaatgcttccacaccgtgacaatcttgggcgtggaggatcggtttaatgcttctctgggcacgtaccatgacctgctcaaaatttttaaagataaagccttcttagacgatgaggccaatcaagatatcttggaagagatcgtatggactttaacgctttttgaggatcaagccatgattgaaagaaggctggtgaagtacgcggacgtgttcgaaaaatccgtccttaaaaagttaaagaaacgccattacacgggctggggacgtctttcccagaagcttattaatgggatcaaagacaaacaaactgggaagacaattctcggctttctgaaagacgacggtgtagccaaccgaaattttatgcagttaattaacgacagctccctggacttcgcaaagattatcaagcatgaacaggaaaaaaccatcaagaacgagtcattggaggaaacgattgcgaacctggcaggcagccccgccattaagaaaggcattcttcagtctattaaaattgtcgatgaaatcgttaagattatgggacagaacccagacaatattgttattgagatggcacgcgagaaccaatccacgatgcaaggaatcaaaaactcccgacagcgtctgcgcaagctcgaggaggtgcataagaacaccgggtccaagattttgaaagaatacaacgtgagtaatacgcagcttcagagcgataggctctatttatacctgctgcaggacggaaaggatatgtacaccggcaaggagttggactacgacaatcttagtcaatatgatattgatcacatcatccctcagtctttcataaaagataactctatcgacaacatagtgctgactacacaagctagtaataggggcaagtcagacaacgtgcccaacatagagattgtgaacaaaatgaagtctttttggtataaacagctcaaaaatggggcaattagccagcgcaaattcgaccatttaaccaaggccgagcgtggcgcactgagcgatttcgataaggcaggctttatcaagcgccagctcgtcgagacacggcagataaccaaacatgtggctcaaatcctggacagtcggttcaattccaatcttacggaggactctaaatctaacagaaacgttaagataataactctcaagtcaaaaatggtgagtgacttccgaaaggactttggcttttacaagctgagagaagtaaatgattatcaccacgcccaggacgcatatctcaatgccgtcgtcggtactgccttacttaagaagtaccctaaactggaagcagagttcgtgtatggggattacaagcactacgatctcgctaagttaatgattcaaccggacagtagccttggaaaagccacaaccagaatgttcttctattctaacctcatgaatttcttcaaaaaagaaatcaaactggccgatgatactatatttacgaggccccagattgaagtgaacaccgaaactggggagattgtctgggataaggtaaaggacatgcagaccatcaggaaagtgatgtcctatccacaagtcaacatagtgatgaaaaccgaagtccagactggggggttttctaaggagagtatcctgcctaagggaaactcagacaaactgatcgcccgcaagaaatcctgggaccctaagaaatacggtggtttcgatagccctatcattgcatattcagtcctggtcgtcgctaagatagccaaaggcaaaacccagaaactcaagactattaaagagttggtcggtatcaaaatcatggagcaggacgaattcgaaaaggatccaattgcgtttctcgaaaagaagggctatcaggacatacagacctcttccatcatcaagctgccgaagtactctctctttgagcttgagaatggacgcaagagactgctggctagcgccaaagaactgcagaagggcaacgaactggccctccctaacaaatacgtaaagttcttgtatttagcatctcattacacaaaattcacaggtaaggaggaagatcgagaaaaaaagcgctcctatgtagagtcacacctgtattactttgacgagattatgcagattatcgttgagtattctaaccggtacattctcgccgacagcaatctgattaaaattcagaacttgtacaaagagaaggataactttagtatcgaggagcaagccattaatatgctcaatctcttcacttttacagatctcggcgcgccagccgctttcaagttctttaacggagatatagatcggaagcggtacagctctaccaacgagatcattaattctactctgatttaccagagtcccacagggttatacgagaccaggatcgacctcagtaagctggggggcaaa

SEQ ID NO: 247 - protein, 1129 aa, Streptococcus lutetiensis
MSNGKILGLDIGVASVGVGIIDAKTGNVIHANSRLFSAANAENNAERRGFRGARRLTRRKKHRVKRVRDLFEKYDISTDFRNLNLNPYELRVKGLTEQLTNEELFAALRTIAKRRGISYLDDAEDDSTGSSDYAKSIDENRRLLKTKTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKKSKALGASKLLKEIAKIVDCSVDDIKGYRVDNKDKPDLHTFEPYRKLKFNLSSIDIDELSRETLDKLADILTLNTEREGIEDTIKRNLPSQFTEEQISEIVQIRKNQSSAFNKGWHSFSAKLMNELIPELYVTSEEQMTILTRLEKFKVNKKSSKNTKTIDEKEITDEIYNPVVAKSVRQTIKIINAAVKKYGDFDKIVIEMPRDKNAEDEKKFIDKKEKENKKEKDDSLKRAAFLYNGTDNLPDGVFHGNKELKTKIRLWYQQGERCLYSGKLISIHDLVHNSNKFEIDHILPLSLSFDDSLANKVLVYAWTNQEKGQKTPYQVIDSMDAAWSFREMKDYVLKQKRLGKKKREYLLTTENIDKIEVKKKFIERNLVDTRYASRVVLNSLQTALKELGKDTKVSVVRGQFTSQLRRKWNIDKSRETYHHHAVDALIIAASSQLKLWQKQENPMFESYGENQVVNKETGEILSISDDKYKELVFQPPYQGFVNTISSKGFEDEILFSYQVDSKFNRKVSDATIYSTRKAKLGKDKKDETYVLGKIKDIYSQDGFDTFIKRYKKDKTQFLMYQKDPLTWENVIEVILRDYPSEKLSEDGKKTVKCNPFEEYRRENGLICKYSKKGNGTPIKSLKYYDKKLGNCIDITPEKSKNRVVLRQISPWRADIYFNLETLKYELMGLKYSDLSFEKGTGKYHISQEKYDAIREKEGIGKKSEFKFTLYRNDLILIKDTLNNCERMLRFGSKNDTSKHYVELKPLEKGTFDSEEEILPVLGKVAKSGQFIKGLNKPNISIYKVRTDVLGNKFFIKKEGDKPKLDFKNNNK

SEQ ID NO: 248 - DNA, 3387 bp, Streptococcus lutetiensis
  source 1..3387: mol_type = genomic DNA
atgtcaaatggcaaaatcttaggcttggatatcggggtggccagcgtcggggttggcataattgatgccaaaaccggcaacgtgatccacgcaaatagcaggctgtttagcgccgccaacgccgagaacaatgctgagcggaggggattccgcggcgcacgtaggctcacgaggcgcaaaaaacatagagtgaagcgggtccgtgacctgtttgaaaagtatgatatctcaacagatttccgcaacttaaatctgaacccctacgagctcagggtgaaaggcctgacagaacagcttaccaatgaagaactcttcgcagctttaagaactattgccaaacggcgcggcatctcctacttggatgacgcggaagacgattctaccggaagcagcgactacgcgaagtcaatcgacgaaaatagacgtcttctgaaaaccaaaactccagggcaaatccagctggagagactggagaagtacggacagctgaggggcaattttaccgtgtatgacgaaaacggagaagctcacagactgatcaatgttttttccacttccgattataaaaacgaagcccggaagatcctggagacgcagagcaactacaacaagcaaatcaccgatgagttcatcgaagattacattgagatattaactcaaaagcgtaaatactaccatggcccaggcaacgagaagagcaggaccgattacggcaggttccgaacagatggaactaccctggagaacatttttggcattcttattggaaaatgctcattctatccagaggaatatcgtgctagtaaggcaagctacaccgcccaagaattcaactttctgaatgacctgaataatctgaaggtccccaccgaaacgggcaagttatcaactgagcagaaggagtatttagtggattttgccaagaagtctaaggctctgggagcgtctaagcttctgaaggagattgccaagatagttgattgcagcgttgacgacatcaaggggtacagggtggataataaagacaagccagatctgcacacctttgagccatatagaaagttgaagtttaacttgagtagtatcgacatcgatgaactgtctagagagacactcgacaaactcgctgacattcttactctgaacacagaacgggaaggcatcgaggatacaatcaaaagaaaccttccctcacagtttaccgaggaacagataagcgagattgtccaaattcggaagaatcaatccagcgcctttaacaagggttggcactccttctcagcaaagttgatgaacgagttaatcccagagctgtacgtgacttcagaggagcagatgacaattctgaccaggttggaaaaatttaaggtgaacaagaagagctccaaaaacacaaagaccatcgatgaaaaggagattactgacgagatctataacccagtcgtcgcgaaatccgtgaggcaaactatcaagattatcaacgccgcggtgaaaaagtatggagactttgacaaaatcgtgattgagatgccacgtgacaagaatgcagaggatgagaaaaaatttattgacaaaaaggagaaggaaaataagaaggaaaaagatgatagcctgaagcgcgcagctttcctgtataacggcacagacaatttgccagacggagtatttcacggaaacaaggagctcaagactaaaattcgcttatggtatcaacaaggcgagaggtgcttgtatagcggcaaactgatatccatacacgacctcgtacacaacagtaacaagtttgagattgaccacatccttccacttagcctgagtttcgacgacagcctggcaaataaggtcttggtatatgcttggaccaatcaggagaaggggcaaaaaaccccgtaccaggtgatagatagcatggacgcggcatggagttttcgggaaatgaaggactacgttctcaaacagaagagactcggcaaaaaaaagcgtgaatacctgctgactaccgagaacattgacaaaatcgaagtcaaaaaaaagttcatcgagcgcaaccttgtggatacccgctatgcctcacgcgtcgtcctgaactctctgcagacagctctgaaagaactgggcaaggacaccaaagtgtctgtcgttaggggtcaatttacctcccagttgcgacgcaagtggaatatcgataagtccagagaaacataccatcatcacgcagtagacgcccttatcattgccgcatcttctcagcttaaactgtggcaaaagcaggaaaatcctatgtttgagtcttatggcgaaaatcaggtcgtcaataaggagacaggagagatcttatcaatatccgatgacaagtataaagaactggtgtttcaaccaccataccaagggtttgtcaacactatcagcagtaaaggcttcgaggatgagatcttgttttcatatcaggtggacagcaaattcaaccggaaagtttctgatgccaccatttatagtactcgcaaagcgaaacttggaaaggacaagaaggatgagacctacgtattggggaaaatcaaggacatttactctcaggacggctttgacaccttcattaagcgttacaaaaaggacaagacgcagttcctgatgtaccaaaaagatccactgacttgggaaaatgttattgaggtgatcctccgggattatccaagtgaaaaattgtcagaggacggcaaaaaaacagtgaagtgcaatccgtttgaagaatataggcgagagaatggtctgatctgtaaatactctaaaaagggcaacggaacccccatcaagtccctgaaatattacgacaagaaacttggtaactgcattgacatcacccctgagaaaagcaagaaccgcgtggtgctgaggcagatatcaccttggcgcgctgatatctacttcaacctggagaccttgaaatatgagctcatgggcttgaaatacagtgacctgtcttttgaaaaagggaccgggaagtatcacattagccaggaaaagtacgatgcgattagagaaaaagaaggcattggcaaaaagagcgagtttaagtttactttgtatcgaaacgatctcatcctgataaaagataccctgaacaattgtgagaggatgcttaggttcggatccaagaacgatacatctaagcactacgtggaactcaaacctttagagaagggcacctttgattccgaggaggagatccttccagtgctgggcaaggttgcgaaatccgggcagtttattaagggtcttaacaaacccaatatctcaatctataaggtgaggaccgatgtgcttggcaacaaattctttatcaagaaggaaggcgacaaacccaagctggatttcaagaataataacaag

SEQ ID NO: 249 - protein, 1345 aa, Streptococcus mutans
MKKPYSIGLDIGTNSVGWAVVTDDYKVPAKKMKVLGNTDKSHIKKNLLGALLFDSGNTAADRRLKRTARRRYTRRRNRILYLQEIFAEEMSKVDDSFFHRLEDSFLVTEDKRGERHPIFGNLEEEVKYYENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEDLEELLGKIGDDYADLFTLAKNLYDAILLSGILTADDSSTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKILTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKPYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHEENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLSKLGGD

SEQ ID NO: 250 - DNA, 4035 bp, Streptococcus mutans
  source 1..4035: mol_type = genomic DNA
atgaagaagccttactcaattggcctggatattggcactaattcagtgggatgggccgtcgttaccgatgattacaaggtacccgcaaagaagatgaaggtccttggtaatacagataaaagtcacataaagaagaatcttctcggagctcttctgttcgacagcgggaacacagctgccgataggcgactcaaaagaactgctcgcaggcgctatacaaggcgcagaaaccgcattctgtacctgcaggagatcttcgccgaagagatgtccaaagtggatgacagttttttccataggctcgaggatagcttcctggtgaccgaggacaaaaggggggagagacatcccattttcggtaatcttgaagaggaggttaagtactacgagaacttcccgactatatatcatctgcggcagtatctcgcagacaaccccgaaaaagtggacctgcgacttgtgtatcttgccctggcacatattataaaattcagaggccactttctgattgaggggaagtttgatacccggaataatgatgtgcagcgcctgtttcaggaattcttggctgtctacgacaatacatttgagaatagtagtttgcaggagcagaacgtgcaagtggaagagatcctgacagacaagatctccaagagcgccaaaaaagatagggtgctcaaattgttccctaatgagaaatccaacggcaggtttgccgaattcttgaaactgattgtgggaaaccaggctgactttaagaaacatttcgagcttgaagaaaaggcccctttgcagttttccaaagacacctacgaggaggatctggaggaactgctgggaaagatcggggatgactatgccgatctgtttaccctcgccaagaacctgtacgatgcgattctcttgtccggtatcctgacggcagacgacagttcaactaaagctccgctctctgccagcatgattcagcgatacaatgagcatcagatggatctggcccagctcaagcagttcatccgacagaaactcagcgataagtacaacgaggtgtttagcgacgtgtccaaagacgggtacgcaggctacattgacggcaagaccaaccaagaagcgttctacaaatacctgaaagggctgctcaacaagatagaaggatcaggttactttctggataaaatcgaacgggaggattttttgcgcaagcagcgaactttcgacaatgggtccatccctcatcagattcacctgcaggaaatgagagctattattaggagacaggctgaattttacccttttctggcagataaccaggatcggatcgagaaaatcttaacctttcggatcccatactatgtgggcccactggcccgtggcaaatccgacttcgcatggctgtcacggaagtccgccgataaaattacgccgtggaactttgatgaaattgtcgataaggaatcttccgctgaggcttttatcaatcgcatgaccaattacgatctgtacctgcctaatcagaaggtgttacccaagcatagcctgttgtatgaaaaattcactgtctacaatgaactcaccaaagtcaagtacaagacagaacagggcaaaaccgcctttttcgacgctaatatgaaacaggaaatttttgacggggtgttcaaagtctatagaaaggtcactaaggacaaactgatggattttctggagaaggaatttgatgagtttcgcatagttgatcttactggtttggataaagaaaataaggtcttcaatgcaagctacggtacataccacgacctttgtaaaattctcgacaaggatttcctcgacaactccaaaaatgaaaagattcttgaggatatcgtgttaaccctgaccctgtttgaagacagggaaatgatccggaagcggctggagaattactccgacctgttgactaaagagcaggtgaaaaagctcgagaggcgccattacaccggatgggggagactcagtgccgaacttatccatggaattcgaaacaaggagagcaggaagaccattctcgattatctgattgacgatggtaatagcaacagaaattttatgcagctgatcaacgatgatgcactgtcatttaaggaggaaattgcaaaagcccaggttatcggcgagaccgacaacctgaatcaggttgtgagtgacatcgcagggagccccgctatcaagaagggaatcctccagtccctcaagattgtcgacgagctcgtcaagatcatggggcatcagccagagaacattgtcgtggagatggcccgcgaaaaccaatttaccaaccaagggaggcggaacagccagcaaagactgaagggcttaacagatagcattaaagagttcggatctcagatacttaaagaacaccccgtcgaaaactcccagttgcagaatgaccgcctctttctgtattatctgcaaaacggaagggacatgtatacgggagaggagctggatatagattaccttagtcaatatgatatcgatcacataatcccccaagcctttatcaaggacaactctatagacaatagggtcctgacctctagcaaagagaatagaggcaagtccgatgacgtaccttctaaggatgtcgtgcgcaagatgaagccatactggagcaagctgctgtctgcaaagcttataacccaacgaaagttcgataatctgactaaggccgagcgcggcgggctgacagatgacgataaggccgggttcattaagcgccagctggtggaaacaagacaaatcactaaacacgtcgctcgaattcttgatgagcggtttaacacagaaacggacgaaaacaacaaaaagatccgccaggtaaaaattgtaaccctgaagagcaaccttgtttctaatttcagaaaggaattcgaactttacaaagtgcgtgaaatcaacgactaccatcatgcccatgacgcttatctgaacgctgtcatcgggaaggccctccttggggtctatcctcagctggagcctgaatttgtgtacggagattacccacactttcacgggcacgaagagaacaaggcaactgctaagaagttcttttattcaaatatcatgaatttttttaagaaagacgacgtcagaactgataaaaacggtgagatcatttggaagaaggacgaacatatcagtaatattaaaaaggtgcttagctatcctcaggtgaacatagttaaaaaagtagaggagcagacaggcgggttctccaaggaatccatactgccaaaaggcaacagcgataaactgatacctcggaagactaaaaaattctactgggataccaagaagtacgggggatttgacagccccattgtcgcctactctatattggttattgcggacatcgaaaagggcaaatccaaaaagcttaaaactgtcaaagccctggtgggggttaccatcatggagaaaatgacctttgaacgcgatcccgtagcattcctcgaacgcaagggctaccgcaacgttcaggaggagaatatcatcaagttgcccaaatattctctctttaagctggagaacggcagaaagcgcctgctcgcatccgcaagggagttacagaaaggcaacgaaattgtactgcccaatcacctcggaaccctgctgtatcacgccaaaaatatccataaagtcgacgaacctaagcacttagactatgtcgataaacacaaggacgaatttaaagagctgctggacgtggttagcaatttctcaaagaaatacacgctggcggaaggtaatctggagaaaattaaagagttgtacgctcagaataacggggaggatcttaaagaactggcgtcctcatttatcaacctgctgaccttcaccgccatcggcgcgcctgctacatttaaattcttcgataagaatatcgatagaaagagatatacttccacgaccgaaattttgaacgctaccctgattcaccaatctattaccgggttatatgagacacgaattgatctgagcaaactggggggggat

SEQ ID NO: 251 - protein, 1368 aa, Streptococcus parauberis
MQKSYSLGLDIGTNSVGWAVITDDYKVPAKKMKVLGNTDRQTVKKNMIGTLLFDSGETAEARRLKRTARRRYTRRINRIKYLQSIFDDEMSKIDSAFFQRIKDSFLVPDDKNDDRHPIFGNIKDEVDYHKNYPTIYHLRKKLADSDEKADLRLIYLALAHIIKFRGHFLIEGDLDSQNTDVNALFLKLVDTYNLMFEDDKIDTQTIDATVILTEKMSKSRRLENLIAKIPNQKKNTLFGNLISLSLGLTPNFKANFELSEDAKLQISKDSFEEDLDNLLAQIGDQYADLFIAAKNLSDAILLSDILTVKGVNTKAPLSASMVQRFNEHQDDLKLLKKLVKVQLPEKYKEIFDIKDKNGYAGYINGKTSQEDFYKYIKPILSKLKGAESLISKLEREDFLRKQRTFDNGSIPHQIHLNELKSIIRRQEKYYPFLKDKQVRIEKIFTFRIPYFVGPLANGNSSFAWVKRRSNESITPWNFEEVVEQEASAKVFIERMTNFDTYLPEEKVLPKHSLLYEMFTVYNELTKVKYQAEGMRKPEFLSSEEKIEIVSNLFKKERKVTVKQLKENYFNKIRCLDSITISGVEDKFNASLGTYHDLLNIIKNQKILDDEQNQDSLEDIVLTLTLFEDEKMIAKRLSKYESIFEPSILKKLKKRHYTGWGRLSQKLINGIRDKQTGKTILDFLIDDGQANRNFMQLINDPSLDFASIIKGAQEKTIKSEKLEETIANLAGSPAIKKGILQSVKIVDEVVKVMGYEPSNIVIEMARENQSTHRGINNSRERLRKLEEVHKNIGSKILKEHEISNAQLQSDRVYLYLLQDGKDMYTGKDLDFDRLSQYDIDHIIPQSFIKDNSIDNIVLTSQESNRGKSDNVPYIAIVNKMKSYWQHQLKSGAISQRKFDNLTKVERGGLSEYDKAGFIKRQLVETRQITKHVAQILNNRFNNNVDNSSKNKRPVKIITLKSKMVSDFRKEFGFYKIREVNDYHHAHDAYLNAVVGTALLKKYPKLEAEFVYGDYKHYDLASLVVKSDTSLGKATAKMFFYSNIMNFFKKEVRLADGTVITRPQIETNTETGEIVWDKVKDIKTIRKVLSIPQINVVKKTEVQTGGFSKESILPKGDSDKLIPRKNNWDPKKYGGFDSPIIAYSVLVVAKVAKGKSQKTKSVKELVGITIMEQNEFEKDRITFLEKKGYQDIQESLIIKLPKFSLFELENGRKRLLASAKELQKGNELSLPNKYIQFLYLASRYTSFSGKEEDREKHRHFVESHLHYFDEIKDIIADFSRRYILADANLEKILTLYNEKNQFSIEEQATNMLNLFTFTGLGAPATLKFFNVDIDRKRYTSSTEILNSTLIRQSITGLYETRIDLSKIGGD

SEQ ID NO: 252 - DNA, 4104 bp, Streptococcus parauberis
  source 1..4104: mol_type = genomic DNA
atgcaaaagagctactctctcgggttagacatcggaacaaatagtgtgggatgggcggtgattacggacgattataaggtgccagccaaaaagatgaaggttcttggcaatacggaccggcagacggtgaagaagaacatgattggcactctgctgtttgatagtggagaaaccgctgaggcccggagactcaaaaggactgctaggcgacggtatacgcggcgtattaaccgcattaaatatcttcagtctatatttgatgatgagatgtcaaagatcgacagcgcgttttttcagcgaattaaagattccttccttgtcccagatgacaagaatgacgatagacatccgatttttggtaacattaaggacgaggttgactaccataagaactatccgacaatttatcacctgcgcaagaagctggcagactccgacgagaaggcagaccttagactgatttacctcgctctggctcacatcataaaatttcgaggacacttcttgatagaaggagatctcgacagccagaatactgatgttaacgccctgttcctgaaattagtcgacacctacaacctcatgtttgaggatgacaaaatcgatacgcagactattgacgcaacagtgattttaactgagaagatgagtaagtcacggcgacttgagaacttgatagccaagatacctaatcaaaagaagaataccctcttcggaaatctgatttcactcagtcttggcctgacacctaactttaaagctaattttgaattgagcgaggacgcgaagcttcaaatctctaaggactccttcgaagaagatttggataacctcctcgcccagatcggtgaccaatacgctgacctgtttatagcagcgaagaatttgtctgacgctatcctcctgtctgatatccttactgtgaagggcgtgaatacaaaggcacccttatccgccagtatggtccagcggttcaacgaacatcaagacgacctgaagttgctcaaaaaactcgtgaaggtgcaactgcccgagaaatacaaagaaattttcgacattaaagacaaaaatgggtacgctgggtatattaacggtaagacatcccaggaggacttttacaaatatatcaagcctatcttaagcaagctgaaaggggcggagtcccttatctctaaattggagagagaagactttttgcggaagcagagaaccttcgataatggatccattccccaccagattcacttgaatgagctcaaatccatcatccgacgacaggagaagtattatccctttctgaaggataaacaggtgcggattgaaaagatcttcacctttagaataccatattttgttggaccattggctaacgggaactcttcatttgcttgggttaagcgaagatctaacgaatctattacaccatggaactttgaggaagtcgttgagcaggaggccagcgccaaggtcttcatagagcggatgactaattttgatacctacctgccagaggagaaggtccttcccaagcactctttgctctatgaaatgttcactgtatacaacgaactgactaaagtaaagtatcaggccgagggcatgagaaagcccgaattcttgagttcagaagaaaagattgagattgtgtccaacctgtttaagaaggagagaaaggtgacagtcaagcagcttaaggaaaattatttcaataagataagatgtcttgactcaatcaccatcagtggggttgaagacaagttcaacgcatcactgggtacttaccacgatttacttaacattattaagaaccagaagattctggacgatgagcagaaccaggactccctcgaggatattgtgttgactctgacactgttcgaggacgaaaaaatgatcgcgaagaggctgtcaaagtatgaatccattttcgagcccagcattttgaagaaattaaaaaagcgccactatactggttggggccgtttatcccagaagctcatcaacggcatccgtgataaacagaccggaaagaccatcctggacttcctgatcgacgatggccaggcgaatcgaaatttcatgcaattgattaacgatccctctctggactttgcgtcaataatcaagggggcccaggaaaagacgataaagagcgagaagctcgaagagaccatcgctaatctcgccggatctcccgctatcaagaaaggcatcttacagtctgtgaagattgtagatgaagtggtgaaagtgatgggctatgaacctagcaacattgtcatagaaatggccagggaaaatcagtcaacccaccgaggcataaataactctagggaacgattacgaaagctggaggaggtccacaagaacattggctccaagatcttgaaagagcacgaaattagcaatgcccaactccagagtgaccgagtgtacttgtatctgttgcaggatggaaaagatatgtacaccggtaaggacctcgatttcgatcggctctctcagtacgatattgatcacatcataccacagtcctttattaaggacaacagtattgataatatcgtcctgacatctcaggaaagcaatagaggaaagtcagataatgtgccctacattgcaatcgtgaataagatgaaatcatactggcaacaccagctgaaatctggggctatcagccagcggaaatttgataatttaactaaggtggagcggggcggcctcagcgagtatgataaggcaggttttatcaaacgtcagctcgttgagacacgtcagataacaaagcacgtggcacaaatccttaataatagattcaacaacaacgtcgataacagtagcaagaacaaaagacctgtcaagataatcacattaaaatctaaaatggtgtctgatttccgtaaggaattcggcttctataaaattagggaggtaaatgactatcatcacgcccacgacgcctacctcaacgccgttgtcgggacagccctgttgaaaaaatatccaaagctggaggcagaattcgtgtacggcgattacaagcactatgacttggcctcactggttgtcaagagcgacactagtctgggcaaagccactgcaaaaatgtttttttattctaatatcatgaacttcttcaaaaaggaggtcagactggcagatggcaccgtgatcacaagacctcagatagagactaatacggaaactggcgagatcgtgtgggataaggtaaaggacattaaaacaattaggaaggtgctgtctataccccagatcaacgtggttaaaaagactgaagtccaaactgggggtttctcaaaggaaagcatcctgcccaagggcgatagcgataagcttattcctagaaagaacaattgggatccaaagaagtatggtggctttgattctccgatcattgcctattctgtcttagtggtcgcaaaagtggcgaagggcaaaagccagaagacaaagagtgtcaaagaacttgtcggaattactatcatggaacagaacgagttcgaaaaggatcggattacattccttgagaaaaaaggataccaggatattcaggaatcactgatcattaagctgcccaagttcagcttgtttgagcttgaaaacgggagaaagcgtctgctcgccagcgcaaaagagctccagaagggaaatgagctgtcattgccaaacaagtacatccaatttttgtatctcgcctccagatatactagctttagcggcaaggaggaagatagagagaagcacagacacttcgtggaatctcacctgcactactttgatgagattaaagacataattgccgatttttctcgacgctatattctggcagatgcgaaccttgaaaaaattctcacgctgtacaatgagaaaaatcagttctcaattgaagagcaggctaccaacatgctgaacctcttcaccttcacgggactgggagcccctgccaccctgaaatttttcaacgtggacattgatcggaagcgatacacttcctccaccgagattctgaatagtaccctcattagacagagtattaccggactctacgagacaaggattgacctctccaaaattggcggggac

SEQ ID NO: 253 - protein, 1750 aa, synthetic construct
MTNGMILGLAIGVASVGVGIIEADSGKVIHASSRIFPSANADNNVERRKFRGSRRLLRRKKHRVKRLQDLFDKYDIVTNFDNLNLNPYELRVKGLNEPLSNEELFASLRNITKHRGISYLDDAEDDSSGNGTEYAKAIELNQQLLKEKTPGQIQYDRLNQYGQLRGNFDIVDENGEIHHVINVFSTSSYRKEAEQILKKQSETNTSISTDFINDFIQLLTSKRKYYHGPGNPKSRTDYGRYRTDGTDLDNIFDVLIGKCSFYPEEYRASKTSYTAQEFNFLNDLNNLTLPTETGKLSEQQKIDLVNWAKETKILGPKKLLQEIAKRNNCKYEDIRGYRLDKKDNPDMHVFDVYRKMNFDLETISVKDLSVDSLNQLARILTLNTEREGIEEAIKNLMPNQFTEKQMLELIAFRKSNSSIFGKGWHSLSIKLMKELIPELYHTSDEQMTILNRFGKFKLTKLDSKRTNYIDENFVTDEIYNPVVAKSVRQAIKIINASIKKWGDFDKIVIEMPRDKNEEEERKRIADGQKVNAKEKEQAEKHAAKLFNGKEELPSEVFHGYKELALRIRLWYQQDQKCLYSGKEITISDLIYNRELFEIDAILPLSLSFDDSLSNKVLVYRWANQEKGQRTPFQALDSMKSAWSYREFKNAILHNSKISRRKRDYFLTEQDISKIEVKQKFIERNLVDTRYASRTVLNVLQQSLKNLEKETKVSVVRGQFTSQLRRKWHIDKTRDTYHHHAVDALIIAASAKLRYWKKQGDILFENYLINRHVDRVTGEIQSDDSYKEEVFTPPYDGFVQTISNPGFEDEILFSYQVDSKVNRKISDATIYATRSAKLEKDKKEQTYVLGKIKDIYSQTGFENFLKIYNKDKSKFLIYQKDPETWEKIIEPILKNYREFDNKGKDIVNPFEKYRNDNGPICKYSRKGNGPEIKQFKYYDTVYKITSGLDISPRESRNKVILQSLNPWRTDFYFNPKTMKYELMGIRYVDLEFEKGTGDYLISDNLYKEIKKNEGISELSVFKFTLYKNDLLLIKDTENNEEQIFRFWSRNDLSSKNRVELKPYDRSRFSGNEILITKMGKAPKQCIKTLTYQNISIYKIKTDILGFKYYLKNEGNKPLLHFKKTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 254 - DNA, 5250 bp, synthetic construct
  source 1..5250: mol_type = other DNA
atgactaatgggatgattctgggcttagcaatcggagtcgcgtctgtaggggtaggaattatcgaggccgatagtggcaaggtaattcacgcaagctcacggatcttccctagtgctaatgccgacaataatgtggagcgccgcaagttccggggatctaggcgccttcttaggaggaaaaagcacagggttaagcgcttacaggatctgtttgacaagtacgatatcgtgactaacttcgataacctcaaccttaacccctacgagctgcgagttaagggcttaaacgagccattgagcaacgaagagctcttcgcatcactccggaacatcacaaagcacaggggcatttcctatctcgatgatgctgaggatgactcttctggaaacgggacagaatacgccaaagcgatagagctgaatcagcagcttctgaaagaaaagacccccggtcagatccagtacgacagactcaatcagtatgggcaactgagaggcaatttcgatatcgtggatgaaaacggcgagattcaccacgtgataaacgttttttcaacatcaagttacagaaaggaagccgagcagattctcaagaagcagtctgaaacgaatactagtatcagcaccgactttataaatgatttcatccaattgctgacctctaagaggaaatattaccatggtcctggtaatccaaagagccgcacagattacgggcgctaccggacggatgggacggatctcgataacatcttcgatgttctgataggtaaatgcagcttttacccagaggagtaccgagccagcaagacgagctacactgcccaagagttcaactttcttaatgacttgaataacctgaccttaccaaccgagacaggcaagttgagcgagcagcagaagatcgacctggtgaattgggctaaggagacaaagatcctcggaccgaaaaagctgcttcaggaaattgccaagaggaacaactgcaagtacgaggacattcgcggctatcggcttgataagaaagataaccccgatatgcatgtatttgatgtgtatcggaagatgaattttgacctggagactatttccgttaaggatctgtcagtcgactctctgaatcagctcgcgcgaattctgacactgaacaccgagagggaggggatcgaagaggccatcaaaaatctgatgccaaaccagttcaccgagaagcaaatgcttgaactcatcgccttccgcaagagtaattcctctatctttgggaaggggtggcacagtctgtcaattaaactgatgaaagagctgatacccgagctctaccacaccagtgacgaacaaatgaccatactcaatcgatttggtaagttcaagctcacgaagctcgactcaaaaaggaccaattacatcgatgaaaactttgtcactgatgaaatctataaccctgtagtggccaagagtgtgaggcaggcaataaagatcatcaacgcttccattaaaaagtggggggactttgataagatcgtgattgagatgccacgcgacaagaatgaggaggaggaaaggaaacgaatcgccgatggccagaaggtgaatgctaaggaaaaagagcaggccgagaagcacgccgcaaagctctttaatggcaaggaagagctcccttctgaagttttccatggatataaggagctggctttgcgaattagactctggtatcagcaagaccagaagtgcctctattctggcaaggagataacaatttcagacctgatctacaacagggagctctttgagattgacgccatccttccgctgtctctttcttttgacgacagtctgtctaacaaggtcctggtttacagatgggcaaatcaggagaagggccagaggacccctttccaagcccttgattccatgaaatcagcgtggtcctatcgggagttcaagaatgcaatcctgcacaattctaaaatcagccggagaaagcgtgactattttctgacagaacaagacattagtaagattgaggtgaaacaaaagtttattgagaggaacttggtggacacacggtacgccagtagaacagttctcaacgtgctgcagcagtccctgaagaatctggagaaggagactaaggtgtccgttgtccgaggacagttcacgtcccagctgcgccggaaatggcacatagataagaccagggatacttaccatcaccatgcggtggacgcactgattatcgcggcctccgctaagttgagatattggaagaaacagggcgacatcttgttcgagaactatctcatcaatcgccacgtagatagagtaaccggggagatacaatctgacgatagctataaggaggaggtgttcacacctccctacgacggatttgtccagactattagcaacccagggtttgaggacgagatccttttctcctatcaggtagacagtaaagtcaacagaaagatctcagacgccacgatatacgctacgaggtctgcgaagctcgagaaggacaagaaggaacagacgtatgtcttgggtaagataaaagatatctattcacaaactggttttgagaacttcctgaagatctataataaggacaagagtaagttcctgatctaccagaaggaccctgagacttgggaaaagatcattgaaccaattctcaaaaattatcgggaattcgataataaaggcaaggatatcgtgaatccatttgagaaatacaggaatgataacgggcctatctgcaagtacagtcggaaaggcaacggccctgagatcaaacaatttaaatactacgacaccgtttacaaaattacaagcggtctcgacatcagcccccgcgaatcaagaaataaggtaattcttcaaagcctgaatccgtggagaaccgacttctactttaaccctaagactatgaagtacgaacttatgggtatcagatatgtcgacctggagttcgagaaaggaacaggggactacctgatttctgacaatctctataaagagattaaaaagaacgaggggatctctgagctgagtgtattcaagttcacactctacaagaacgatctcctgctgatcaaggacactgagaacaacgaagagcaaatttttaggttttggtctcggaatgacctgtcctccaaaaaccgggtggaactgaagccctacgataggtcccgcttttccggcaatgagatccttatcaccaaaatgggcaaggcacctaagcaatgcattaagactttaacataccaaaacatctccatttataaaatcaaaacagacatcctgggattcaaatactatctgaaaaacgaaggaaataagccattactgcactttaagaagaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 255 - protein, 1998 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 256 - DNA, 5994 bp, synthetic construct
  source 1..5994: mol_type = other DNA
atggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 257 - protein, 1697 aa, synthetic construct
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 258 - DNA, 5091 bp, synthetic construct
  source 1..5091: mol_type = other DNA
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 259 - protein, 2005 aa, synthetic construct
MNKPYSIGLAIGTNSVGWSIITDDYKVPAKKMRVLGNTDKEYIKKNLIGALLFDGGNTAADRRLKRTARRRYTRRRNRILYLQEIFAEEMSKVDDSFFHRLEDSFLVEEDKRGSKYPIFATMQEEKYYHEKFPTIYHLRKELADKKEKADLRLVYLALAHIIKFRGHFLIEDDRFDVRNTDIQKQYQAFLEIFDTTFENNHLLSQNVDVEAILTDKISKSAKKDRILAQYPNQKSTGIFAEFLKLIVGNQADFKKHFNLEDKTPLQFAKDSYDEDLENLLGQIGDEFADLFSVAKKLYDSVLLSGILTVTDLSTKAPLSASMIQRYDEHHEDLKHLKQFVKASLPENYREVFADSSKDGYAGYIEGKTNQEAFYKYLLKLLTKQEGSEYFLEKIKNEDFLRKQRTFDNGSIPHQVHLTELRAIIRRQSEYYPFLKENQDRIEKILTFRIPYYVGPLAREKSDFAWMTRKTDDSIRPWNFEDLVDKEKSAEAFIHRMTNNDLYLPEEKVLPKHSLIYEKFTVYNELTKVRFLAEGFKDFQFLNRKQKETIFNSLFKEKRKVTEKDIISFLNKVDGYEGIAIKGIEKQFNASLSTYHDLKKILGKDFLDNTDNELILEDIVQTLTLFEDREMIKKCLDIYKDFFTESQLKKLYRRHYTGWGRLSAKLINGIRNKENQKTILDYLIDDGSANRNFMQLINDDDLSFKPIIDKARTGSHSDNLKEVVGELAGSPAIKKGILQSLKIVDELVKVMGYEPEQIVVEMARENQTTAKGLSRSRQRLTTLRESLANLKSNILEEKKPKYVKDQVENHHLSDDRLFLYYLQNGRDMYTKKALDIDNLSQYDIDAIIPQAFIKDDSIDNRVLVSSAKNRGKSDDVPSIEIVKARKMFWKNLLDAKLMSQRKYDNLTKAERGGLTSDDKARFIQRQLVETRQITKHVARILDERFNNEVDNGKKICKVKIVTLKSNLVSNFRKEFGFYKIREVNDYHHAHDAYLNAVVAKAILTKYPQLEPEFVYGMYRQKKLSKIVHEDKEEKYSEATRKMFFYSNLMNMFKRVVRLADGSIVVRPVIETGRYMGKTAWDKKKHFATVRKVLSYPQNNIVKKTEIQTGGFSKESILAHGNSDKLIPRKTKDIYLDPKKYGGFDSPIVAYSVLVVADIKKGKAQKLKTVTELLGITIMERSRFEKNPSAFLESKGYLNIRDDKLMILPKYSLFELENGRRRLLASAGELQKGNELALPTQFMKFLYLASRYNESKGKPEEIEKKQEFVNQHVSYFDDILQLINDFSKRVILADANLEKINKLYQDNKENIPVDELANNIINLFTFTSLGAPAAFKFFDKIVDRKRYTSTKEVLNSTLIHQSITGLYETRIDLGKLGEDTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 260 - DNA, 6015 bp, synthetic construct
  source 1..6015: mol_type = other DNA
atgaacaagccttattcaataggattagctatagggacaaattctgtggggtggagtataatcaccgacgattacaaggtgcctgcaaagaagatgcgcgtgctcggcaatacagacaaggaatatattaagaagaacctgatcggggccctcctttttgacggtggcaacacagcagctgaccgccgcctcaagaggaccgctcggagacggtatactcgccggcgtaatcggatcctgtatttgcaggaaatttttgctgaagaaatgtctaaggtggatgattcattctttcaccggctcgaagactcctttctggtggaggaagacaagaggggctcaaagtacccaatcttcgccacaatgcaagaagagaaatactaccacgagaagtttcccacaatctatcatctcaggaaagagctggccgataaaaaagagaaggccgatttgcgactggtttacttggccttggcacacatcataaagttccggggacactttctgattgaagacgaccgttttgacgtccgcaacactgatatacagaagcaataccaagcgttccttgagatctttgacaccacatttgaaaacaaccatctgctgagccaaaatgtggacgtggaagccattctgactgataagatctctaaatctgccaaaaaggacagaatccttgcccagtaccccaaccagaagtcaactggcattttcgccgagtttctgaagttgatagttggcaatcaggccgattttaagaagcacttcaatttggaggacaaaacgcctctccaattcgccaaggactcatatgatgaggacctggagaatctgcttggccaaatcggggatgagttcgctgatctttttagcgtggcaaagaagctctatgactctgtactcctgagcggaatcctgacagttaccgatctttcaacaaaggcacccctgagtgcaagcatgattcaacgctacgacgagcaccatgaggatctgaaacatctgaagcagttcgtcaaggcttctctgcctgaaaactatcgggaggtcttcgccgactcatctaaggacggctacgccggatacatcgagggaaagacaaatcaggaggctttctacaagtacctgttgaagctgcttacaaaacaggaggggagcgaatacttcctggagaagatcaaaaacgaggacttcctgcgtaaacagaggactttcgataatggctccattcctcaccaggtgcatctcacggaactgagagctatcattagacgtcagagtgagtattacccatttctgaaggagaaccaagaccgaatcgaaaaaattctgacgttccggatcccttactatgtcggacctttagctagggaaaaaagtgacttcgcctggatgacccgaaagacagatgatagtatcagaccatggaactttgaagacctggtggacaaagagaagagcgccgaggcttttattcacaggatgaccaataatgatctctatctgcctgaagagaaggtgctgcccaaacacagtctcatctacgaaaaatttacagtctataacgaactgacaaaggtccgctttctggctgaaggattcaaggactttcaatttctgaaccggaagcagaaggaaactatctttaactcattgtttaaggaaaagaggaaggttaccgaaaaagacatcatctcctttttaaacaaggtagatgggtacgaagggattgccattaaaggcattgagaaacagtttaacgccagcctttcaacctaccatgatctcaagaagatcctcggaaaagatttccttgacaataccgacaacgaacttatcctggaggatatagtgcagacactcactctgttcgaggacagggaaatgataaagaagtgcctcgacatatataaagacttctttaccgagagtcaactgaaaaagttgtatagaaggcattacaccggttggggccgactgagtgcaaaactcattaacggcatccggaataaggagaatcaaaagactatcctcgattacctcatcgatgacggaagcgcaaacagaaacttcatgcaactcatcaacgatgatgacctgtctttcaaaccaattatagacaaagccaggactgggagccatagtgacaatctgaaggaagtggtgggagagctggcaggcagccccgcaattaagaaggggatcctgcagagcctcaaaattgtcgatgaactcgtgaaggtcatgggctatgaacctgaacagattgttgtagagatggcccgagagaaccagactactgcgaagggacttagccggagcagacaacgactgaccactttgcgagagagtctggcgaacctgaagtctaatattctcgaggaaaaaaagccaaagtacgtgaaggaccaggtggagaatcaccacctgagcgacgacagactctttctgtattatctgcagaacggcagagatatgtatacgaagaaggcactggacatagacaacctgagtcagtatgacatcgatgccattatccctcaggccttcatcaaagacgattcaatcgacaatcgcgtacttgttagcagtgcgaaaaaccggggaaagtctgatgacgtcccatccatcgaaatagtgaaggcaaggaagatgttctggaagaatctgctggatgccaaattaatgtcacaacggaagtacgacaacctgacaaaggcagaaagggggggcttaacaagcgacgataaggcaaggtttatccagaggcagttggtcgagaccaggcaaatcaccaaacacgtcgcccggatcctggatgaacgcttcaacaatgaagtcgacaatggcaaaaaaatctgtaaagtcaagatagtgacactgaagtcaaatctggtgagcaacttccggaaagaattcggcttctataaaattcgcgaagtgaacgactatcaccatgcgcacgacgcttacctgaatgcagtcgtggcgaaagccattttgaccaagtacccccagctggagcctgagtttgtgtacggaatgtaccgacaaaagaagctgagcaagattgtacacgaggataaggaagagaaatactccgaggccactcggaagatgttcttctactctaatctgatgaacatgtttaagagagtggtgaggttggcagacggctccattgttgtaaggccagtgatcgagactgggcgatacatgggcaagacagcgtgggacaagaagaagcatttcgcaaccgtacggaaagtcctgtcctacccgcagaataacattgtgaagaagacagaaatacaaaccggtggtttctcaaaagagtccattttagcccatggcaacagtgacaaattgattccacggaagaccaaagatatttatctggaccctaaaaaatacggcggattcgactcaccgatcgtggcatacagcgtattggtggtggccgatattaagaagggtaaagcccagaaactcaagactgttaccgagctcctgggtatcactataatggagagaagccggtttgagaagaaccctagcgcctttttggaatccaaggggtatctgaacattcgggacgataagctgatgatcttgcctaaatacagcctttttgaactggagaatggacgaaggcgcctgcttgcctcagcgggggaactgcagaaaggcaatgagctggcccttcctacccagttcatgaaatttttgtatctggctagtaggtataacgagtcaaaaggcaagccagaggagatcgaaaagaagcaggaatttgtaaaccagcatgtgtcatactttgatgatatcctgcagttaatcaatgacttcagtaaacgagtcattctcgcagacgccaacttggagaaaattaataagctgtaccaggacaacaaagagaatataccagtcgacgagcttgcaaataacattattaacctgttcacttttacatccctgggggcccctgctgcgttcaaatttttcgacaaaatcgtggatcgaaagcgatatacatccactaaggaagttctgaacagcactctcatccaccagtctatcactggcctttacgaaacgcgtattgacttggggaaactcggagaggacaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 261 - protein, 1973 aa, synthetic construct
MKKPYSIGLAIGTNSVGWAVVTDDYKVPAKKMKVLGNTDKSHIKKNLLGALLFDSGNTAADRRLKRTARRRYTRRRNRILYLQEIFAEEMSKVDDSFFHRLEDSFLVTEDKRGERHPIFGNLEEEVKYYENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEDLEELLGKIGDDYADLFTLAKNLYDAILLSGILTADDSSTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKILTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDAIIPQAFIKDNSIDNRVLTSSKENRGKSDDVPSKDVVRKMKPYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHEENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLSKLGGDTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 262 - DNA, 5919 bp, synthetic construct
  source 1..5919: mol_type = other DNA
atgaagaagccttactcaattggcctggctattggcactaattcagtgggatgggccgtcgttaccgatgattacaaggtacccgcaaagaagatgaaggtccttggtaatacagataaaagtcacataaagaagaatcttctcggagctcttctgttcgacagcgggaacacagctgccgataggcgactcaaaagaactgctcgcaggcgctatacaaggcgcagaaaccgcattctgtacctgcaggagatcttcgccgaagagatgtccaaagtggatgacagttttttccataggctcgaggatagcttcctggtgaccgaggacaaaaggggggagagacatcccattttcggtaatcttgaagaggaggttaagtactacgagaacttcccgactatatatcatctgcggcagtatctcgcagacaaccccgaaaaagtggacctgcgacttgtgtatcttgccctggcacatattataaaattcagaggccactttctgattgaggggaagtttgatacccggaataatgatgtgcagcgcctgtttcaggaattcttggctgtctacgacaatacatttgagaatagtagtttgcaggagcagaacgtgcaagtggaagagatcctgacagacaagatctccaagagcgccaaaaaagatagggtgctcaaattgttccctaatgagaaatccaacggcaggtttgccgaattcttgaaactgattgtgggaaaccaggctgactttaagaaacatttcgagcttgaagaaaaggcccctttgcagttttccaaagacacctacgaggaggatctggaggaactgctgggaaagatcggggatgactatgccgatctgtttaccctcgccaagaacctgtacgatgcgattctcttgtccggtatcctgacggcagacgacagttcaactaaagctccgctctctgccagcatgattcagcgatacaatgagcatcagatggatctggcccagctcaagcagttcatccgacagaaactcagcgataagtacaacgaggtgtttagcgacgtgtccaaagacgggtacgcaggctacattgacggcaagaccaaccaagaagcgttctacaaatacctgaaagggctgctcaacaagatagaaggatcaggttactttctggataaaatcgaacgggaggattttttgcgcaagcagcgaactttcgacaatgggtccatccctcatcagattcacctgcaggaaatgagagctattattaggagacaggctgaattttacccttttctggcagataaccaggatcggatcgagaaaatcttaacctttcggatcccatactatgtgggcccactggcccgtggcaaatccgacttcgcatggctgtcacggaagtccgccgataaaattacgccgtggaactttgatgaaattgtcgataaggaatcttccgctgaggcttttatcaatcgcatgaccaattacgatctgtacctgcctaatcagaaggtgttacccaagcatagcctgttgtatgaaaaattcactgtctacaatgaactcaccaaagtcaagtacaagacagaacagggcaaaaccgcctttttcgacgctaatatgaaacaggaaatttttgacggggtgttcaaagtctatagaaaggtcactaaggacaaactgatggattttctggagaaggaatttgatgagtttcgcatagttgatcttactggtttggataaagaaaataaggtcttcaatgcaagctacggtacataccacgacctttgtaaaattctcgacaaggatttcctcgacaactccaaaaatgaaaagattcttgaggatatcgtgttaaccctgaccctgtttgaagacagggaaatgatccggaagcggctggagaattactccgacctgttgactaaagagcaggtgaaaaagctcgagaggcgccattacaccggatgggggagactcagtgccgaacttatccatggaattcgaaacaaggagagcaggaagaccattctcgattatctgattgacgatggtaatagcaacagaaattttatgcagctgatcaacgatgatgcactgtcatttaaggaggaaattgcaaaagcccaggttatcggcgagaccgacaacctgaatcaggttgtgagtgacatcgcagggagccccgctatcaagaagggaatcctccagtccctcaagattgtcgacgagctcgtcaagatcatggggcatcagccagagaacattgtcgtggagatggcccgcgaaaaccaatttaccaaccaagggaggcggaacagccagcaaagactgaagggcttaacagatagcattaaagagttcggatctcagatacttaaagaacaccccgtcgaaaactcccagttgcagaatgaccgcctctttctgtattatctgcaaaacggaagggacatgtatacgggagaggagctggatatagattaccttagtcaatatgatatcgatgctataatcccccaagcctttatcaaggacaactctatagacaatagggtcctgacctctagcaaagagaatagaggcaagtccgatgacgtaccttctaaggatgtcgtgcgcaagatgaagccatactggagcaagctgctgtctgcaaagcttataacccaacgaaagttcgataatctgactaaggccgagcgcggcgggctgacagatgacgataaggccgggttcattaagcgccagctggtggaaacaagacaaatcactaaacacgtcgctcgaattcttgatgagcggtttaacacagaaacggacgaaaacaacaaaaagatccgccaggtaaaaattgtaaccctgaagagcaaccttgtttctaatttcagaaaggaattcgaactttacaaagtgcgtgaaatcaacgactaccatcatgcccatgacgcttatctgaacgctgtcatcgggaaggccctccttggggtctatcctcagctggagcctgaatttgtgtacggagattacccacactttcacgggcacgaagagaacaaggcaactgctaagaagttcttttattcaaatatcatgaatttttttaagaaagacgacgtcagaactgataaaaacggtgagatcatttggaagaaggacgaacatatcagtaatattaaaaaggtgcttagctatcctcaggtgaacatagttaaaaaagtagaggagcagacaggcgggttctccaaggaatccatactgccaaaaggcaacagcgataaactgatacctcggaagactaaaaaattctactgggataccaagaagtacgggggatttgacagccccattgtcgcctactctatattggttattgcggacatcgaaaagggcaaatccaaaaagcttaaaactgtcaaagccctggtgggggttaccatcatggagaaaatgacctttgaacgcgatcccgtagcattcctcgaacgcaagggctaccgcaacgttcaggaggagaatatcatcaagttgcccaaatattctctctttaagctggagaacggcagaaagcgcctgctcgcatccgcaagggagttacagaaaggcaacgaaattgtactgcccaatcacctcggaaccctgctgtatcacgccaaaaatatccataaagtcgacgaacctaagcacttagactatgtcgataaacacaaggacgaatttaaagagctgctggacgtggttagcaatttctcaaagaaatacacgctggcggaaggtaatctggagaaaattaaagagttgtacgctcagaataacggggaggatcttaaagaactggcgtcctcatttatcaacctgctgaccttcaccgccatcggcgcgcctgctacatttaaattcttcgataagaatatcgatagaaagagatatacttccacgaccgaaattttgaacgctaccctgattcaccaatctattaccgggttatatgagacacgaattgatctgagcaaactggggggggataccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 263 - protein, 1758 aa, synthetic construct
MTNGKILGLAIGIASVGVGIIEAKTGKVVHANSRLFSAANAENNAERRGFRGSRRLNRRKKHRVKRVRDLFEKYEIVTDFRNLNLNPYELRVKGLTEQLTNEELFAALRTISKRRGISYLDDAEDDSTGSTDYAKSIDENRRLLKTKTPGQIQLERLEKYGQLRGNFTVYDENGEAHRLINVFSTSDYEKEARKILETQADYNKKITAEFIDDYVEILTQKRKYYHGPGNEKSRTDYGRFRTDGTTLENIFGILIGKCSFYPDEYRASKASYTAQEYNFLNDLNNLKVPTETGKLSTEQKEALVEFAKSTATLGPAKLLKEIAKILDCKVDEIKGYREDDKGKPDLHTFEPYRKLKFNLDSVNIDDLSREVLDKLADILTLNTEREGIEDAIRHNLPNQFTEGQISEIIKVRKSQSTAFNKGWHSFSAKLMNELIPELYATSDEQMTILTRLEKFKVNKKSSKNTKTIDEKEVTDEIYNPVVAKSVRQTIKIINAAVKKYGDFDKIVIEMPRDKNADDEKKFIDKRNKENKKEKDDALKRAAYLYNGTDKLPDEVFHGNKQLETKIRLWYQQGERCLYSGKPIPIQELVHNSNNFEIDAILPLSLSFDDSLANKVLVYAWTNQEKGQKTPYQVIDSMDAAWSFREMKDYVLKQKGLGKKKRDYLLTTENIDKIEVKKKFIERNLVDTRYASRVVLNSLQSALRELGKDTKISVIRGQFTSQLRRKWKIDKSRETYHHHAVDALIIAASSQLKLWEKQDNPMFVDYGNNQVVDKQTGEILSVSDDEYKELVFQPPYQGFVNMISSKGFEDEILFSYQVDSKYNRKVSDATIYSTRKAKIGKDKKEETYVLGKIKDIYSQNGFDTFIKKYNKDKTQFLMYQKDPLTWENVIEVILRDYPTTKKSEDGKNDVKCNPFEEYRRENGLVCKYSKKGKGTPIKSLKYYDKKLGNCIDITPEGSKNEVVLQSLNPWRADVYFNPETLKYELLGLKYSDLSFEKGTGKYHISQEKYDVIKEKEGIGKKSEFKFTLYRNDLILIKDTASGEQEIYRFLSRTMPNVKHYAELKPYDKEKFDNVQELVEALGEADKVGRCIKGLNKSNLSIYKVRTDVLGNKYFVKKEGDKPKLDFKNNKKTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 264 - DNA, 5274 bp, synthetic construct
  source 1..5274: mol_type = other DNA
atgacaaacggcaaaattctgggtctggccatcggaatcgctagcgttggcgtgggaatcattgaagcgaagacaggtaaagtcgtccatgcaaattctcgattgttctccgcagctaacgctgaaaacaatgcggagagaaggggtttcagaggctctaggcggctcaaccggcgcaagaagcacagggtaaaaagagtgcgagatctctttgagaaatatgagatcgtgactgattttagaaacctgaatctgaacccatatgagctgagagtgaaaggacttacggaacagctcactaatgaagagttgttcgccgccctgcggaccatcagcaaacgccgaggaatttcctaccttgatgacgccgaagatgacagtaccggtagcacagattatgccaagagcattgatgagaacaggagactgctgaagactaagacacctggacagatacaattggaacggctcgagaagtacggccagctgaggggtaacttcaccgtttatgacgaaaatggggaggcccatagactgataaatgtgttctcaacttctgactatgaaaaggaggcccggaaaatcctcgagactcaagccgactacaacaagaagattacagccgagtttattgacgattacgtggaaattttaacccagaaaaggaagtattaccacgggccaggaaatgaaaagagccgcaccgactatgggagattcagaacggatggaacaaccttagagaatatctttggaatccttattggtaaatgctctttctatcctgacgagtatcgcgccagcaaagcctcctataccgctcaggagtacaacttcttgaatgatttgaacaatttgaaggttccgacggagactggcaagctgagtaccgagcaaaaggaggcccttgtggaattcgccaagtctactgcaacattaggtcctgctaaacttctgaaggagattgccaaaattttggactgcaaagtcgatgaaatcaaggggtaccgtgaggatgataaagggaaaccagacctgcacacctttgagccctatagaaagttgaaattcaatctggacagcgtcaacattgacgatttgagtcgcgaagtgctggacaagctggcagacattttgacacttaacactgaaagggagggcattgaggatgccatcaggcataacctgcccaaccaatttactgagggccagatctccgaaatcatcaaggtgcgcaaaagccagagcactgctttcaacaaggggtggcacagcttctctgccaagctcatgaacgaattgattcccgagctctatgccacaagcgacgaacagatgactatacttactcggctggagaaatttaaggtcaataaaaaatcctccaaaaacaccaagacgattgacgagaaagaggtcactgatgaaatctacaatccagttgtagccaagtctgtccggcaaacgatcaagatcattaacgctgctgtgaagaaatatggagactttgataagattgtgattgaaatgcctcgcgacaagaatgcggacgatgagaagaagtttatcgataagagaaacaaagaaaataagaaagaaaaggatgatgccctgaagcgggcagcttacctttataatggaaccgataagctgccagatgaggtgtttcacggaaacaagcaacttgaaaccaagattcgcctgtggtaccagcagggagaacggtgtttgtactcaggcaagcctatcccaatccaggagttggtccacaactccaataacttcgaaatcgatgcgattctgcccctgtccctgagttttgacgactccctggccaacaaggtgcttgtgtatgcttggaccaaccaagagaagggccagaagacgccctaccaggtgattgattctatggatgcggcgtggtcctttcgcgagatgaaggactatgtgctcaagcaaaaaggcctcggcaaaaagaaacgggattatcttttgaccaccgagaacattgacaagattgaagtgaagaaaaaattcatcgagcgcaacttggtcgataccagatatgcctctagggttgtgctgaactcactgcagtctgctttgagagagctgggtaaagacactaaaattagtgtaatcaggggccagttcacaagtcagcttaggcggaaatggaagatcgacaagtcacgcgagacatatcatcatcacgcagtcgacgcactgataattgcagcttcaagtcagctcaagttgtgggagaaacaggataaccctatgtttgtcgactatggaaacaatcaggtcgtcgataagcagaccggggaaattttaagtgtgtccgatgacgagtataaggagcttgtctttcagccaccgtaccagggctttgtcaacatgattagtagcaagggttttgaggacgaaattttgttcagctaccaggtcgattccaaatacaatagaaaagtatccgacgcaaccatatattctactcgcaaggccaagattggcaaagataagaaggaagagacctatgtattggggaagatcaaagacatttactcacaaaatggattcgacaccttcattaagaagtacaacaaagataagacacagtttttgatgtaccagaaagatccactgacatgggaaaacgtgatcgaagttatactgcgtgactaccccacgactaaaaagagtgaggacggaaaaaacgacgtgaagtgcaacccgtttgaagaataccggagagaaaacggtctggtgtgtaagtactctaagaaaggaaaggggacccctattaaatccctcaaatactacgacaaaaaactcgggaactgcatcgatatcaccccggaaggttccaaaaatgaagtcgtgcttcaatccttgaatccgtggagggcagatgtgtactttaacccagaaaccttgaagtatgaattactgggacttaaatacagtgatctctcatttgaaaagggcactggaaaataccatatctctcaggagaagtacgacgtcattaaggaaaaagaagggatcgggaaaaaatccgagttcaagttcacattgtataggaacgacctgatccttattaaagacacagccagcggtgagcaggagatttaccgatttctgtctagaaccatgcctaacgtcaagcactatgcggagctgaagccctatgacaaagaaaaatttgataacgtccaggaactcgtcgaggcgctgggcgaagccgacaaggtaggccgctgtataaaggggctgaacaaaagcaacctcagcatctataaagttaggacagatgtgctcgggaacaaatacttcgttaagaaggaaggggacaagcccaagctggattttaagaacaataaaaagaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 265 - protein, 1996 aa, synthetic construct
MRKPYSIGLAIGTNSVGWAVITDDYKVPSKKMRIQGTTDRTSIKKNLIGALLFDNGETAEATRLKRTTRRRYTRRKYRIKELQKIFSSEMNELDIAFFPRLSESFLVSDDKEFENHPIFGNLKDEITYHNDYPTIYHLRQTLADRDQKADLRLIYLALAHIIKFRGHFLIEGNLDSENTDVHVLFLNLVNIYNNLFEEDIVETASIDAEKILTSKTSKSRRLENLIAEIPNQKRNMLFGNLVSLALGLTPNFKTNFELLEDAKLQISKDSYEEDLDNLLAQIGDQYADLFIAAKKLSDAILLSDIITVKGASTKAPLSASMVQRYEEHQQDLALLKNLVKKQIPEKYKEIFDNKEKNGYAGYIDGKTSQEEFYKYIKPILLKLNGTEKLISKLEREDFLRKQRTFDNGSIPHQIHLNELKAIIRRQEKFYPFLKENQKKIEKLFTFKIPYYVGPLANGQSSFAWLKRQSNESITPWNFEEVVDQEASARAFIERMTNFDTYLPEEKVLPKHSPLYEMFMVYNELTKVKYQTEGMKRPVFLSSEDKEEIVNLLFKKDRKVTVKQLKEEYFSKMKCFHTVTILGVEDRFNASLGTYHDLLKIFKDKAFLDDEANQDILEEIVWTLTLFEDQAMIERRLVKYADVFEKSVLKKLKKRHYTGWGRLSQKLINGIKDKQTGKTILGFLKDDGVANRNFMQLINDSSLDFAKIIKHEQEKTIKNESLEETIANLAGSPAIKKGILQSIKIVDEIVKIMGQNPDNIVIEMARENQSTMQGIKNSRQRLRKLEEVHKNTGSKILKEYNVSNTQLQSDRLYLYLLQDGKDMYTGKELDYDNLSQYDIDAIIPQSFIKDNSIDNIVLTTQASNRGKSDNVPNIEIVNKMKSFWYKQLKNGAISQRKFDHLTKAERGALSDFDKAGFIKRQLVETRQITKHVAQILDSRFNSNLTEDSKSNRNVKIITLKSKMVSDFRKDFGFYKLREVNDYHHAQDAYLNAVVGTALLKKYPKLEAEFVYGDYKHYDLAKLMIQPDSSLGKATTRMFFYSNLMNFFKKEIKLADDTIFTRPQIEVNTETGEIVWDKVKDMQTIRKVMSYPQVNIVMKTEVQTGGFSKESILPKGNSDKLIARKKSWDPKKYGGFDSPIIAYSVLVVAKIAKGKTQKLKTIKELVGIKIMEQDEFEKDPIAFLEKKGYQDIQTSSIIKLPKYSLFELENGRKRLLASAKELQKGNELALPNKYVKFLYLASHYTKFTGKEEDREKKRSYVESHLYYFDEIMQIIVEYSNRYILADSNLIKIQNLYKEKDNFSIEEQAINMLNLFTFTDLGAPAAFKFFNGDIDRKRYSSTNEIINSTLIYQSPTGLYETRIDLSKLGGKTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 266 - DNA, 5988 bp, synthetic construct
  source 1..5988: mol_type = other DNA
atgcgcaaaccttactcaattggcctggcaatcgggactaattctgttggctgggctgtgattactgatgattacaaggtgccaagtaagaaaatgaggattcagggcacgactgatcggaccagcattaagaagaatctcattggggccctcctgttcgataatggcgagactgccgaggccactcgattaaagagaacaacaaggaggaggtacaccagacggaagtaccgaataaaggaactgcaaaagatcttcagcagcgaaatgaatgagctcgacattgcttttttccctagactgtctgagagttttcttgtgagtgacgacaaagaattcgagaatcatccgatttttggaaaccttaaagatgagataacttatcataacgattaccctactatttatcacttgcgacagacacttgcagaccgtgaccagaaggccgatcttaggctcatttatctcgctctggcccacattattaaatttcgggggcactttttgatcgaaggcaatctggacagtgagaacacggacgtacacgtgctgtttctgaacctggtgaacatatataataacctgttcgaggaagatatagttgaaaccgcatccatagacgctgagaagattcttacctcaaaaacttccaaatccaggcggctcgagaatcttatagctgagattcctaaccagaagcggaacatgttgtttggcaacctcgtgtctctggctctcggcctgacaccaaattttaaaaccaattttgagctgctggaggatgcaaagttacagatctccaaggattcatatgaagaagacctcgacaacttgttggcacagattggggatcagtacgcagatctctttatcgccgctaaaaagctttctgacgcaatattactgtctgacatcatcaccgtgaagggcgcctccactaaagcgcctctttcagcatccatggtgcagagatatgaagagcatcaacaggacctcgctctcctgaagaatctcgtgaaaaaacagattcctgagaagtataaggaaatcttcgataacaaggagaagaatggctatgcaggttatatcgatggcaagacctcccaggaggaattttacaagtacatcaagcccatacttcttaagctcaacggcacagagaagttgatcagcaaacttgagcgggaggacttcctgagaaagcaacgaacattcgacaacggatctattcctcaccagattcacctgaatgagctcaaggcaatcatccggaggcaggagaagttttatccctttctgaaggaaaatcagaagaaaatcgaaaagcttttcacatttaaaattccctattacgtcgggccactcgccaatggccagagtagcttcgcctggctgaagagacagtccaacgagtctatcaccccctggaacttcgaggaagtggtggatcaagaggcctcagcgcgcgccttcatagagaggatgactaacttcgatacctatttacccgaggagaaggttctgccaaagcacagcccactctacgaaatgtttatggtctataatgagctcaccaaggttaagtatcagaccgaggggatgaagaggcccgtctttctctcttccgaagacaaagaagaaatagtgaatctcctgttcaaaaaagaccggaaggtcactgtcaagcagctgaaggaggaatatttctccaaaatgaaatgcttccacaccgtgacaatcttgggcgtggaggatcggtttaatgcttctctgggcacgtaccatgacctgctcaaaatttttaaagataaagccttcttagacgatgaggccaatcaagatatcttggaagagatcgtatggactttaacgctttttgaggatcaagccatgattgaaagaaggctggtgaagtacgcggacgtgttcgaaaaatccgtccttaaaaagttaaagaaacgccattacacgggctggggacgtctttcccagaagcttattaatgggatcaaagacaaacaaactgggaagacaattctcggctttctgaaagacgacggtgtagccaaccgaaattttatgcagttaattaacgacagctccctggacttcgcaaagattatcaagcatgaacaggaaaaaaccatcaagaacgagtcattggaggaaacgattgcgaacctggcaggcagccccgccattaagaaaggcattcttcagtctattaaaattgtcgatgaaatcgttaagattatgggacagaacccagacaatattgttattgagatggcacgcgagaaccaatccacgatgcaaggaatcaaaaactcccgacagcgtctgcgcaagctcgaggaggtgcataagaacaccgggtccaagattttgaaagaatacaacgtgagtaatacgcagcttcagagcgataggctctatttatacctgctgcaggacggaaaggatatgtacaccggcaaggagttggactacgacaatcttagtcaatatgatattgatgcgatcatccctcagtctttcataaaagataactctatcgacaacatagtgctgactacacaagctagtaataggggcaagtcagacaacgtgcccaacatagagattgtgaacaaaatgaagtctttttggtataaacagctcaaaaatggggcaattagccagcgcaaattcgaccatttaaccaaggccgagcgtggcgcactgagcgatttcgataaggcaggctttatcaagcgccagctcgtcgagacacggcagataaccaaacatgtggctcaaatcctggacagtcggttcaattccaatcttacggaggactctaaatctaacagaaacgttaagataataactctcaagtcaaaaatggtgagtgacttccgaaaggactttggcttttacaagctgagagaagtaaatgattatcaccacgcccaggacgcatatctcaatgccgtcgtcggtactgccttacttaagaagtaccctaaactggaagcagagttcgtgtatggggattacaagcactacgatctcgctaagttaatgattcaaccggacagtagccttggaaaagccacaaccagaatgttcttctattctaacctcatgaatttcttcaaaaaagaaatcaaactggccgatgatactatatttacgaggccccagattgaagtgaacaccgaaactggggagattgtctgggataaggtaaaggacatgcagaccatcaggaaagtgatgtcctatccacaagtcaacatagtgatgaaaaccgaagtccagactggggggttttctaaggagagtatcctgcctaagggaaactcagacaaactgatcgcccgcaagaaatcctgggaccctaagaaatacggtggtttcgatagccctatcattgcatattcagtcctggtcgtcgctaagatagccaaaggcaaaacccagaaactcaagactattaaagagttggtcggtatcaaaatcatggagcaggacgaattcgaaaaggatccaattgcgtttctcgaaaagaagggctatcaggacatacagacctcttccatcatcaagctgccgaagtactctctctttgagcttgagaatggacgcaagagactgctggctagcgccaaagaactgcagaagggcaacgaactggccctccctaacaaatacgtaaagttcttgtatttagcatctcattacacaaaattcacaggtaaggaggaagatcgagaaaaaaagcgctcctatgtagagtcacacctgtattactttgacgagattatgcagattatcgttgagtattctaaccggtacattctcgccgacagcaatctgattaaaattcagaacttgtacaaagagaaggataactttagtatcgaggagcaagccattaatatgctcaatctcttcacttttacagatctcggcgcgccagccgctttcaagttctttaacggagatatagatcggaagcggtacagctctaccaacgagatcattaattctactctgatttaccagagtcccacagggttatacgagaccaggatcgacctcagtaagctggggggcaaaaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 267 - protein, 1757 aa, synthetic construct
MSNGKILGLAIGVASVGVGIIDAKTGNVIHANSRLFSAANAENNAERRGFRGARRLTRRKKHRVKRVRDLFEKYDISTDFRNLNLNPYELRVKGLTEQLTNEELFAALRTIAKRRGISYLDDAEDDSTGSSDYAKSIDENRRLLKTKTPGQIQLERLEKYGQLRGNFTVYDENGEAHRLINVFSTSDYKNEARKILETQSNYNKQITDEFIEDYIEILTQKRKYYHGPGNEKSRTDYGRFRTDGTTLENIFGILIGKCSFYPEEYRASKASYTAQEFNFLNDLNNLKVPTETGKLSTEQKEYLVDFAKKSKALGASKLLKEIAKIVDCSVDDIKGYRVDNKDKPDLHTFEPYRKLKFNLSSIDIDELSRETLDKLADILTLNTEREGIEDTIKRNLPSQFTEEQISEIVQIRKNQSSAFNKGWHSFSAKLMNELIPELYVTSEEQMTILTRLEKFKVNKKSSKNTKTIDEKEITDEIYNPVVAKSVRQTIKIINAAVKKYGDFDKIVIEMPRDKNAEDEKKFIDKKEKENKKEKDDSLKRAAFLYNGTDNLPDGVFHGNKELKTKIRLWYQQGERCLYSGKLISIHDLVHNSNKFEIDAILPLSLSFDDSLANKVLVYAWTNQEKGQKTPYQVIDSMDAAWSFREMKDYVLKQKRLGKKKREYLLTTENIDKIEVKKKFIERNLVDTRYASRVVLNSLQTALKELGKDTKVSVVRGQFTSQLRRKWNIDKSRETYHHHAVDALIIAASSQLKLWQKQENPMFESYGENQVVNKETGEILSISDDKYKELVFQPPYQGFVNTISSKGFEDEILFSYQVDSKFNRKVSDATIYSTRKAKLGKDKKDETYVLGKIKDIYSQDGFDTFIKRYKKDKTQFLMYQKDPLTWENVIEVILRDYPSEKLSEDGKKTVKCNPFEEYRRENGLICKYSKKGNGTPIKSLKYYDKKLGNCIDITPEKSKNRVVLRQISPWRADIYFNLETLKYELMGLKYSDLSFEKGTGKYHISQEKYDAIREKEGIGKKSEFKFTLYRNDLILIKDTLNNCERMLRFGSKNDTSKHYVELKPLEKGTFDSEEEILPVLGKVAKSGQFIKGLNKPNISIYKVRTDVLGNKFFIKKEGDKPKLDFKNNNKTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 268 - DNA, 5271 bp, synthetic construct
  source 1..5271: mol_type = other DNA
atgtcaaatggcaaaatcttaggcttggccatcggggtggccagcgtcggggttggcataattgatgccaaaaccggcaacgtgatccacgcaaatagcaggctgtttagcgccgccaacgccgagaacaatgctgagcggaggggattccgcggcgcacgtaggctcacgaggcgcaaaaaacatagagtgaagcgggtccgtgacctgtttgaaaagtatgatatctcaacagatttccgcaacttaaatctgaacccctacgagctcagggtgaaaggcctgacagaacagcttaccaatgaagaactcttcgcagctttaagaactattgccaaacggcgcggcatctcctacttggatgacgcggaagacgattctaccggaagcagcgactacgcgaagtcaatcgacgaaaatagacgtcttctgaaaaccaaaactccagggcaaatccagctggagagactggagaagtacggacagctgaggggcaattttaccgtgtatgacgaaaacggagaagctcacagactgatcaatgttttttccacttccgattataaaaacgaagcccggaagatcctggagacgcagagcaactacaacaagcaaatcaccgatgagttcatcgaagattacattgagatattaactcaaaagcgtaaatactaccatggcccaggcaacgagaagagcaggaccgattacggcaggttccgaacagatggaactaccctggagaacatttttggcattcttattggaaaatgctcattctatccagaggaatatcgtgctagtaaggcaagctacaccgcccaagaattcaactttctgaatgacctgaataatctgaaggtccccaccgaaacgggcaagttatcaactgagcagaaggagtatttagtggattttgccaagaagtctaaggctctgggagcgtctaagcttctgaaggagattgccaagatagttgattgcagcgttgacgacatcaaggggtacagggtggataataaagacaagccagatctgcacacctttgagccatatagaaagttgaagtttaacttgagtagtatcgacatcgatgaactgtctagagagacactcgacaaactcgctgacattcttactctgaacacagaacgggaaggcatcgaggatacaatcaaaagaaaccttccctcacagtttaccgaggaacagataagcgagattgtccaaattcggaagaatcaatccagcgcctttaacaagggttggcactccttctcagcaaagttgatgaacgagttaatcccagagctgtacgtgacttcagaggagcagatgacaattctgaccaggttggaaaaatttaaggtgaacaagaagagctccaaaaacacaaagaccatcgatgaaaaggagattactgacgagatctataacccagtcgtcgcgaaatccgtgaggcaaactatcaagattatcaacgccgcggtgaaaaagtatggagactttgacaaaatcgtgattgagatgccacgtgacaagaatgcagaggatgagaaaaaatttattgacaaaaaggagaaggaaaataagaaggaaaaagatgatagcctgaagcgcgcagctttcctgtataacggcacagacaatttgccagacggagtatttcacggaaacaaggagctcaagactaaaattcgcttatggtatcaacaaggcgagaggtgcttgtatagcggcaaactgatatccatacacgacctcgtacacaacagtaacaagtttgagattgacgccatccttccacttagcctgagtttcgacgacagcctggcaaataaggtcttggtatatgcttggaccaatcaggagaaggggcaaaaaaccccgtaccaggtgatagatagcatggacgcggcatggagttttcgggaaatgaaggactacgttctcaaacagaagagactcggcaaaaaaaagcgtgaatacctgctgactaccgagaacattgacaaaatcgaagtcaaaaaaaagttcatcgagcgcaaccttgtggatacccgctatgcctcacgcgtcgtcctgaactctctgcagacagctctgaaagaactgggcaaggacaccaaagtgtctgtcgttaggggtcaatttacctcccagttgcgacgcaagtggaatatcgataagtccagagaaacataccatcatcacgcagtagacgcccttatcattgccgcatcttctcagcttaaactgtggcaaaagcaggaaaatcctatgtttgagtcttatggcgaaaatcaggtcgtcaataaggagacaggagagatcttatcaatatccgatgacaagtataaagaactggtgtttcaaccaccataccaagggtttgtcaacactatcagcagtaaaggcttcgaggatgagatcttgttttcatatcaggtggacagcaaattcaaccggaaagtttctgatgccaccatttatagtactcgcaaagcgaaacttggaaaggacaagaaggatgagacctacgtattggggaaaatcaaggacatttactctcaggacggctttgacaccttcattaagcgttacaaaaaggacaagacgcagttcctgatgtaccaaaaagatccactgacttgggaaaatgttattgaggtgatcctccgggattatccaagtgaaaaattgtcagaggacggcaaaaaaacagtgaagtgcaatccgtttgaagaatataggcgagagaatggtctgatctgtaaatactctaaaaagggcaacggaacccccatcaagtccctgaaatattacgacaagaaacttggtaactgcattgacatcacccctgagaaaagcaagaaccgcgtggtgctgaggcagatatcaccttggcgcgctgatatctacttcaacctggagaccttgaaatatgagctcatgggcttgaaatacagtgacctgtcttttgaaaaagggaccgggaagtatcacattagccaggaaaagtacgatgcgattagagaaaaagaaggcattggcaaaaagagcgagtttaagtttactttgtatcgaaacgatctcatcctgataaaagataccctgaacaattgtgagaggatgcttaggttcggatccaagaacgatacatctaagcactacgtggaactcaaacctttagagaagggcacctttgattccgaggaggagatccttccagtgctgggcaaggttgcgaaatccgggcagtttattaagggtcttaacaaacccaatatctcaatctataaggtgaggaccgatgtgcttggcaacaaattctttatcaagaaggaaggcgacaaacccaagctggatttcaagaataataacaagaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 269 - protein, 1996 aa, synthetic construct
MQKSYSLGLAIGTNSVGWAVITDDYKVPAKKMKVLGNTDRQTVKKNMIGTLLFDSGETAEARRLKRTARRRYTRRINRIKYLQSIFDDEMSKIDSAFFQRIKDSFLVPDDKNDDRHPIFGNIKDEVDYHKNYPTIYHLRKKLADSDEKADLRLIYLALAHIIKFRGHFLIEGDLDSQNTDVNALFLKLVDTYNLMFEDDKIDTQTIDATVILTEKMSKSRRLENLIAKIPNQKKNTLFGNLISLSLGLTPNFKANFELSEDAKLQISKDSFEEDLDNLLAQIGDQYADLFIAAKNLSDAILLSDILTVKGVNTKAPLSASMVQRFNEHQDDLKLLKKLVKVQLPEKYKEIFDIKDKNGYAGYINGKTSQEDFYKYIKPILSKLKGAESLISKLEREDFLRKQRTFDNGSIPHQIHLNELKSIIRRQEKYYPFLKDKQVRIEKIFTFRIPYFVGPLANGNSSFAWVKRRSNESITPWNFEEVVEQEASAKVFIERMTNFDTYLPEEKVLPKHSLLYEMFTVYNELTKVKYQAEGMRKPEFLSSEEKIEIVSNLFKKERKVTVKQLKENYFNKIRCLDSITISGVEDKFNASLGTYHDLLNIIKNQKILDDEQNQDSLEDIVLTLTLFEDEKMIAKRLSKYESIFEPSILKKLKKRHYTGWGRLSQKLINGIRDKQTGKTILDFLIDDGQANRNFMQLINDPSLDFASIIKGAQEKTIKSEKLEETIANLAGSPAIKKGILQSVKIVDEVVKVMGYEPSNIVIEMARENQSTHRGINNSRERLRKLEEVHKNIGSKILKEHEISNAQLQSDRVYLYLLQDGKDMYTGKDLDFDRLSQYDIDAIIPQSFIKDNSIDNIVLTSQESNRGKSDNVPYIAIVNKMKSYWQHQLKSGAISQRKFDNLTKVERGGLSEYDKAGFIKRQLVETRQITKHVAQILNNRFNNNVDNSSKNKRPVKIITLKSKMVSDFRKEFGFYKIREVNDYHHAHDAYLNAVVGTALLKKYPKLEAEFVYGDYKHYDLASLVVKSDTSLGKATAKMFFYSNIMNFFKKEVRLADGTVITRPQIETNTETGEIVWDKVKDIKTIRKVLSIPQINVVKKTEVQTGGFSKESILPKGDSDKLIPRKNNWDPKKYGGFDSPIIAYSVLVVAKVAKGKSQKTKSVKELVGITIMEQNEFEKDRITFLEKKGYQDIQESLIIKLPKFSLFELENGRKRLLASAKELQKGNELSLPNKYIQFLYLASRYTSFSGKEEDREKHRHFVESHLHYFDEIKDIIADFSRRYILADANLEKILTLYNEKNQFSIEEQATNMLNLFTFTGLGAPATLKFFNVDIDRKRYTSSTEILNSTLIRQSITGLYETRIDLSKIGGDTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 270 - DNA, 5988 bp, synthetic construct
  source 1..5988: mol_type = other DNA
atgcaaaagagctactctctcgggttagcaatcggaacaaatagtgtgggatgggcggtgattacggacgattataaggtgccagccaaaaagatgaaggttcttggcaatacggaccggcagacggtgaagaagaacatgattggcactctgctgtttgatagtggagaaaccgctgaggcccggagactcaaaaggactgctaggcgacggtatacgcggcgtattaaccgcattaaatatcttcagtctatatttgatgatgagatgtcaaagatcgacagcgcgttttttcagcgaattaaagattccttccttgtcccagatgacaagaatgacgatagacatccgatttttggtaacattaaggacgaggttgactaccataagaactatccgacaatttatcacctgcgcaagaagctggcagactccgacgagaaggcagaccttagactgatttacctcgctctggctcacatcataaaatttcgaggacacttcttgatagaaggagatctcgacagccagaatactgatgttaacgccctgttcctgaaattagtcgacacctacaacctcatgtttgaggatgacaaaatcgatacgcagactattgacgcaacagtgattttaactgagaagatgagtaagtcacggcgacttgagaacttgatagccaagatacctaatcaaaagaagaataccctcttcggaaatctgatttcactcagtcttggcctgacacctaactttaaagctaattttgaattgagcgaggacgcgaagcttcaaatctctaaggactccttcgaagaagatttggataacctcctcgcccagatcggtgaccaatacgctgacctgtttatagcagcgaagaatttgtctgacgctatcctcctgtctgatatccttactgtgaagggcgtgaatacaaaggcacccttatccgccagtatggtccagcggttcaacgaacatcaagacgacctgaagttgctcaaaaaactcgtgaaggtgcaactgcccgagaaatacaaagaaattttcgacattaaagacaaaaatgggtacgctgggtatattaacggtaagacatcccaggaggacttttacaaatatatcaagcctatcttaagcaagctgaaaggggcggagtcccttatctctaaattggagagagaagactttttgcggaagcagagaaccttcgataatggatccattccccaccagattcacttgaatgagctcaaatccatcatccgacgacaggagaagtattatccctttctgaaggataaacaggtgcggattgaaaagatcttcacctttagaataccatattttgttggaccattggctaacgggaactcttcatttgcttgggttaagcgaagatctaacgaatctattacaccatggaactttgaggaagtcgttgagcaggaggccagcgccaaggtcttcatagagcggatgactaattttgatacctacctgccagaggagaaggtccttcccaagcactctttgctctatgaaatgttcactgtatacaacgaactgactaaagtaaagtatcaggccgagggcatgagaaagcccgaattcttgagttcagaagaaaagattgagattgtgtccaacctgtttaagaaggagagaaaggtgacagtcaagcagcttaaggaaaattatttcaataagataagatgtcttgactcaatcaccatcagtggggttgaagacaagttcaacgcatcactgggtacttaccacgatttacttaacattattaagaaccagaagattctggacgatgagcagaaccaggactccctcgaggatattgtgttgactctgacactgttcgaggacgaaaaaatgatcgcgaagaggctgtcaaagtatgaatccattttcgagcccagcattttgaagaaattaaaaaagcgccactatactggttggggccgtttatcccagaagctcatcaacggcatccgtgataaacagaccggaaagaccatcctggacttcctgatcgacgatggccaggcgaatcgaaatttcatgcaattgattaacgatccctctctggactttgcgtcaataatcaagggggcccaggaaaagacgataaagagcgagaagctcgaagagaccatcgctaatctcgccggatctcccgctatcaagaaaggcatcttacagtctgtgaagattgtagatgaagtggtgaaagtgatgggctatgaacctagcaacattgtcatagaaatggccagggaaaatcagtcaacccaccgaggcataaataactctagggaacgattacgaaagctggaggaggtccacaagaacattggctccaagatcttgaaagagcacgaaattagcaatgcccaactccagagtgaccgagtgtacttgtatctgttgcaggatggaaaagatatgtacaccggtaaggacctcgatttcgatcggctctctcagtacgatattgatgcaatcataccacagtcctttattaaggacaacagtattgataatatcgtcctgacatctcaggaaagcaatagaggaaagtcagataatgtgccctacattgcaatcgtgaataagatgaaatcatactggcaacaccagctgaaatctggggctatcagccagcggaaatttgataatttaactaaggtggagcggggcggcctcagcgagtatgataaggcaggttttatcaaacgtcagctcgttgagacacgtcagataacaaagcacgtggcacaaatccttaataatagattcaacaacaacgtcgataacagtagcaagaacaaaagacctgtcaagataatcacattaaaatctaaaatggtgtctgatttccgtaaggaattcggcttctataaaattagggaggtaaatgactatcatcacgcccacgacgcctacctcaacgccgttgtcgggacagccctgttgaaaaaatatccaaagctggaggcagaattcgtgtacggcgattacaagcactatgacttggcctcactggttgtcaagagcgacactagtctgggcaaagccactgcaaaaatgtttttttattctaatatcatgaacttcttcaaaaaggaggtcagactggcagatggcaccgtgatcacaagacctcagatagagactaatacggaaactggcgagatcgtgtgggataaggtaaaggacattaaaacaattaggaaggtgctgtctataccccagatcaacgtggttaaaaagactgaagtccaaactgggggtttctcaaaggaaagcatcctgcccaagggcgatagcgataagcttattcctagaaagaacaattgggatccaaagaagtatggtggctttgattctccgatcattgcctattctgtcttagtggtcgcaaaagtggcgaagggcaaaagccagaagacaaagagtgtcaaagaacttgtcggaattactatcatggaacagaacgagttcgaaaaggatcggattacattccttgagaaaaaaggataccaggatattcaggaatcactgatcattaagctgcccaagttcagcttgtttgagcttgaaaacgggagaaagcgtctgctcgccagcgcaaaagagctccagaagggaaatgagctgtcattgccaaacaagtacatccaatttttgtatctcgcctccagatatactagctttagcggcaaggaggaagatagagagaagcacagacacttcgtggaatctcacctgcactactttgatgagattaaagacataattgccgatttttctcgacgctatattctggcagatgcgaaccttgaaaaaattctcacgctgtacaatgagaaaaatcagttctcaattgaagagcaggctaccaacatgctgaacctcttcaccttcacgggactgggagcccctgccaccctgaaatttttcaacgtggacattgatcggaagcgatacacttcctccaccgagattctgaatagtaccctcattagacagagtattaccggactctacgagacaaggattgacctctccaaaattggcggggacaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 271 - protein, 1999 aa, synthetic construct
MDKKYSIGLAIGTNSVGWAVITDDYKVPSKKFKVLGNTDRHSIKKNLIGALLFDSGETAEATRLKRTARRRYTRRKNRIRYLQEIFSSEMSKVDDSFFHRLEESFLVEEDKKHERHPIFGNIVDEVAYHEKYPTIYHLRKKLADSTDKADLRLIYLALAHMIKFRGHFLIEGDLNPDNSDMDKLFIQLVQTYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPEFLSGKQKEAIVDLLFKTNRKVTVKQLKEDYFKKIECFDSVEISGVEDRFNASLGTYHDLLKIIKDKDFLDNEENEDILEDIVLTLTLFEDKEMIEERLKKYANLFDDKVMKQLKRRHYTGWGRLSRKLINGIRDKQSGKTILDFLKSDGFANRNFMQLINDDSLTFKEAIQKAQVSGQGHSLHEQIANLAGSPAIKKGILQSVKVVDELVKVMGHKPENIVIEMARENQTTQKGQKNSRERMKRIEEGIKELGSQILKEHPVENTQLQNEKLYLYYLQNGRDMYVDQELDINRLSDYDVDAIVPQSFIKDDSIDNKILTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYTKLESEFVYGDYKVYDVRKMIAKSEQEIGKATAKRFFYSNIMNFFKTEITLANGEIRKRPLIETNEETGEIVWDKGRDFATVRKVLSMPQVNIVKKTEVQTGALTNESIYARGSFDKLISRKHRFESSKYGGFGSPTVTYSVLVVAKSKVQDGKVKKIKTGKELIGMTLLDKLVFEKNPLKFIEDKGYGNVQIDKCIKLPKYSLFEFENGTRRMLASVMANNNSRGDLQKANEMFLPAKLVTLLYHAHKIESSKELEHEAYILDHYNDLYQLLSYIERFASLYVDVEKNISKVKELFSNIESYSISEICSSVINLLTLTASGAPADFKFLGTTIPRKRYGSPQSILSSTLIHQSITGLYETRIDLSQLGGDTGPKKKRKVASIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 272 - DNA, 5997 bp, synthetic construct
  source 1..5997: mol_type = other DNA
atggataagaagtactccattggactggcaattgggacaaattcagtgggatgggctgtgataacggatgattataaagtgccatctaagaagtttaaagtgctggggaacacagacagacactcaatcaaaaagaatttgattggggccctcctcttcgactcaggtgagaccgctgaagctactcgcctcaagagaacagcgagacggcggtatactcgtagaaagaaccgcattcgctacctgcaagagatatttagcagcgaaatgagtaaggtggacgatagcttcttccacagactggaggagagctttcttgtggaggaggacaagaaacacgagcgccatcccatctttggtaatattgtggacgaggtggcctatcatgagaagtatccaacaatttaccaccttagaaagaagttggcagattccaccgacaaagctgacctccggctgatctaccttgctctcgcacatatgattaaattccggggacacttcttgattgaaggcgaccttaaccccgacaactcagatatggacaagctcttcatccagctcgtacaaacctacaatcagcttttcgaggaaaacccaattaacgcttccagggtcgacgcaaaagcgatactgtctgctcgtcttagtaagtcccggcggctcgagaacttaattgcacagttgcccggcgaaaagcgtaatggactgtttgggaatctcattgccctttcccttggactgactccaaatttcaagtcaaatttcgatctcgctgaggacgcaaaactgcagctgtctaaggacacttacgacgatgacctggacaacctgctggctcagattggcgaccagtacgccgatttattcctcgccgcaaaaaacctttctgatgccatcctgctgagcgatattcttagagttaacagtgagattacaaaagcccccctgagtgcctccatgattaagcgctatgacgaacaccaccaagacttgactctcctgaaagctttagtacggcaacagctccccgagaaatataaggagatctttttcgatcaatccaagaacggatacgcgggatatatagatggaggggctagccaagaggaattttacaagttcatcaaaccaattttagaaaagatggacggaacagaagaattattggccaagctgaatcgggaggatctgctgagaaagcagagaacattcgataacggctccataccccaccagatccacctcggagaattacacgcaattcttagacgccaggaggatttctaccccttcctgaaagacaatcgagagaagattgaaaaaatactgacatttcggatcccctattacgtgggtcctctggcccgagggaatagtcggttcgcctggatgacacgtaagtcagaagagacgattaccccctggaattttgaggaagtggttgataaaggcgccagcgctcagtctttcatcgagcgtatgactaattttgacaaaaacttgcccaacgagaaagtcctccccaaacactccttactttatgagtacttcaccgtctataacgagcttacaaaagttaagtacgtaactgagggtatgaggaaaccagagttcctcagcgggaaacaaaaggaggccattgtggatctgcttttcaaaacaaacaggaaggttaccgtgaaacaattaaaggaggattactttaagaaaatcgagtgcttcgatagcgtcgagatatctggagtagaagacaggttcaacgcgtccctgggtacctaccacgatctgctgaaaataatcaaggacaaggacttcctcgataatgaggaaaatgaagatatcctggaggacatcgtgcttactctgacactgtttgaagacaaagagatgatagaggagaggctgaagaaatatgcaaatcttttcgatgataaagttatgaaacagcttaagcgaaggcattacaccgggtgggggaggctgagccggaagcttatcaatgggatcagggacaagcagagcgggaagactatattggattttctgaagtctgatgggtttgcaaataggaacttcatgcagctcattaatgacgattcactgacatttaaggaggctattcagaaggctcaagtaagtggacaggggcatagcctgcacgaacagattgctaatctcgccggatctccagcaattaagaagggcatcctgcagagtgttaaagttgtggacgagctggtcaaggtgatgggccacaagcctgaaaatatagttattgagatggcgagggaaaaccaaacaactcagaaaggacaaaaaaactcccgcgaacgaatgaaaaggatcgaagagggcattaaagaattgggctcccagattctcaaagaacatcctgttgaaaatacccagctgcagaacgagaagctgtatctgtattatctgcagaacgggagagatatgtacgtcgaccaggagctggacattaaccgattgtctgactacgatgtcgacgcaatcgttccgcaaagcttcataaaggatgattccatcgacaataaaattctcactcggagcgacaaaaatcgaggaaagtctgacaatgtgcccagcgaagaggtggtaaagaagatgaagaactactggagacagcttctgaatgctaaactgattactcaacgtaagttcgacaatctgacaaaggctgaaagggggggtctgagcgagctggataaggctgggttcattaaaaggcagttggtcgaaacccgacaaatcaccaagcatgttgctcagatcttggactcaagaatgaacacaaaatatgatgaaaacgataaactgattagggaggtgaaggtgatcactcttaagagcaagttagtctcagacttcaggaaagattttcagttctataaggtgcgggagattaacaactatcatcatgcccacgacgcgtatctcaacgcggttgtgggaaccgccctgatcaaaaagtacactaagctggagagcgagtttgtttatggagattataaagtgtacgacgtaaggaagatgatcgcgaagtcagagcaggagatcggtaaagctaccgcaaagcgcttcttctacagtaacattatgaacttcttcaagacagagattacgctcgccaatggcgagatacggaagagacccctgattgagactaacgaagaaacaggcgagatcgtttgggacaaaggaagagatttcgctacagtgcggaaagtgctctctatgccccaggtgaatatcgtcaagaagacagaagtgcagaccggagcgttaaccaacgagagcatatatgcacgcggctcctttgataagctgatctccaggaagcacaggttcgagtcctccaagtacgggggcttcggcagcccaactgttacttactccgtcctggtggtggccaaaagcaaagtccaagacgggaaggtcaaaaagatcaagacagggaaagagctgattggcatgacactgttggacaagttggtgttcgagaaaaaccccctgaaatttatagaagacaaggggtacggaaacgtgcagatcgataagtgcattaagctgcctaagtactctttattcgagttcgaaaacggcacccgtcggatgttagcctccgtcatggcgaataataacagcaggggcgacttgcagaaagctaacgaaatgtttctgcctgccaagttggtgacattgctgtatcacgcccacaagattgaatcaagcaaagagctggagcacgaggcatacatccttgatcattacaatgatttgtatcagctcctgtcttacatcgaacggttcgccagcctgtatgtggacgtagagaagaacatatctaaggtaaaggagttgttttccaacatcgaatcctacagcatcagtgagatctgctcctctgtgattaatctcttaactttaacagctagcggggccccggccgactttaaattcttaggtacaacgatcccgcgcaagaggtacggctccccccaatcaattctctccagcacactgattcaccagagcatcaccggcttatatgaaacgaggattgacctgagtcagcttggtggcgacaccggtcctaagaaaaagcggaaagtggctagcattttcaaaccagaagaactacgacaggcactgatgccaactttggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggac

SEQ ID NO: 273-282 - skipped (10 entries)

SEQ ID NO: 283 - DNA, 20 bp, synthetic construct
  source 1..20: mol_type = other DNA
acagggggccagaacttcgg

SEQ ID NO: 284 - DNA, 20 bp, synthetic construct
  source 1..20: mol_type = other DNA
caaaaaatctctgggtccag

SEQ ID NO: 285 - DNA, 20 bp, synthetic construct
  source 1..20: mol_type = other DNA
gacggcagccttctcctcag

SEQ ID NO: 286 - DNA, 20 bp, synthetic construct
  source 1..20: mol_type = other DNA
tagctctcttaaggagtgca

SEQ ID NO: 287 - DNA, 20 bp, synthetic construct
  source 1..20: mol_type = other DNA
catggatcgaattgaataca

SEQ ID NO: 288 - DNA, 20 bp, synthetic construct
  source 1..20: mol_type = other DNA
gactcctcgttgtttacccc

SEQ ID NO: 289 - DNA, 20 bp, synthetic construct
  source 1..20: mol_type = other DNA
attaccctagcaagttgatt

SEQ ID NO: 290 - DNA, 20 bp, synthetic construct
  source 1..20: mol_type = other DNA
tattaccctagcaagttgat

SEQ ID NO: 291 - DNA, 20 bp, synthetic construct
  source 1..20: mol_type = other DNA
cttcggcagtaaagaataaa

SEQ ID NO: 292 - DNA, 20 bp, synthetic construct
  source 1..20: mol_type = other DNA
gctagtgattgcagctgtgt

SEQ ID NO: 293 - DNA, 20 bp, synthetic construct
  source 1..20: mol_type = other DNA
tgcagatagatgaggagcca

SEQ ID NO: 294 - DNA, 20 bp, synthetic construct
  source 1..20: mol_type = other DNA
gcagatagatgaggagccaa

SEQ ID NO: 295 - DNA, 20 bp, synthetic construct
  source 1..20: mol_type = other DNA
cgacaggtttccaaagctgt

SEQ ID NO: 296 - DNA, 20 bp, synthetic construct
  source 1..20: mol_type = other DNA
tcagatacaaaattagagat

SEQ ID NO: 297 - DNA, 20 bp, synthetic construct
  source 1..20: mol_type = other DNA
cagatacaaaattagagatg

SEQ ID NO: 298 - DNA, 20 bp, synthetic construct
  source 1..20: mol_type = other DNA
ttaccctagcaagttgattg

SEQ ID NO: 299 - DNA, 20 bp, synthetic construct
  source 1..20: mol_type = other DNA
cccctgttctccatggtact

SEQ ID NO: 300 - DNA, 20 bp, synthetic construct
  source 1..20: mol_type = other DNA
agggggccagaacttcggca

SEQ ID NO: 301 - DNA, 20 bp, synthetic construct
  source 1..20: mol_type = other DNA
agatagatgaggagccaaca

SEQ ID NO: 302 - DNA, 20 bp, synthetic construct
  source 1..20: mol_type = other DNA
agaacttcggcagtaaagaa

SEQ ID NO: 303 - DNA, 20 bp, synthetic construct
  source 1..20: mol_type = other DNA
caatgcatgggaatgaaggg

SEQ ID NO: 304 - DNA, 20 bp, synthetic construct
  source 1..20: mol_type = other DNA
ttcccaatcaacttgctagg

SEQ ID NO: 305 - DNA, 20 bp, synthetic construct
  source 1..20: mol_type = other DNA
gcttgaggttgtccatgttt

SEQ ID NO: 306 - DNA, 20 bp, synthetic construct
  source 1..20: mol_type = other DNA
aagcaagaagagagccccag

SEQ ID NO: 307 - DNA, 20 bp, synthetic construct
  source 1..20: mol_type = other DNA
ttcaggcacatggatcgaat